SEQUENCE LISTING (2 sequences)

SEQ ID NO: 1 - protein, 30 aa, Artificial Sequence
  Synthetic Polypeptide
MQIPQAPWPVVWAVLQLGWRPGWFLDSPDR

SEQ ID NO: 2 - protein, 23 aa, Artificial Sequence
  Synthetic Polypeptide
MRANDALQVLGLLFSLARGSEVG

(19) United States
(12) Patent Application Publication
(10) Pub. No.: US 2023/0286973 A1
(43) Pub. Date: Sep. 14, 2023

(54) PROTEIN SECRETION INHIBITORS

(71) Applicant: KEZAR LIFE SCIENCES, South San Francisco, CA (US)

(72) Inventors: Dustin McMinn, Pacifica, CA (US); Meera Rao, San Francisco, CA (US)

(21) Appl. No.: 18/019,885

(22) PCT Filed: Aug. 31, 2021
(86) PCT No.: PCT/US21/48317

§ 371 Date: Feb. 6, 2023

Related U.S. Application Data
(60) Provisional application No. 63/072,690, filed on Aug. 31, 2020.

(51) Int. Cl. C07D 417/14; C07D 265/14; C07D 471/04

(52) U.S. Cl. CPC C07D 417/14; C07D 265/14; C07D 471/04

(57) ABSTRACT

Provided herein are secretion inhibitors, such as inhibitors of Sec61 for example of Formula (I), methods for their preparation, related pharmaceutical compositions, and method for using the same.

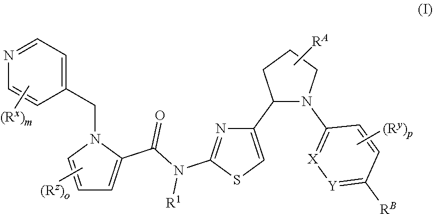

### BACKGROUND

### Field of the Invention

The present disclosure relates to protein secretion inhibitors, including methods of making and using the same.

### INCORPORATION BY REFERENCE OF MATERIAL SUBMITTED ELECTRONICALLY

This application contains, as a separate part of the disclosure, a sequence listing in computer-readable form (filename: 40064PC_Seqlisting.txt; 912 bytes; created: Aug. 11, 2021) which is incorporated by reference in its entirety.

### Description of Related Technology

Protein translocation into the endoplasmic reticulum (“ER”) constitutes the first step of protein secretion. ER protein import is essential in all eukaryotic cells and is particularly important in fast-growing tumor cells. Thus, the process of protein secretion can serve as a target both for potential cancer drugs and for bacterial virulence factors. See Kalies and Römisch, Traffic, 16(10):1027-1038 (2015).

Protein transport to the ER is initiated in the cytosol when N-terminal hydrophobic signal peptides protrude from the ribosome. Binding of signal recognition particle (“SRP”) to the signal sequence allows targeting of the ribosome-nascent chain-SRP complex to the ER membrane where contact of SRP with its receptor triggers handing over of the signal peptide to Sec61. Sec61 is an ER membrane protein translocator (aka translocon) that is doughnut-shaped with 3 major subunits (heterotrimeric). It includes a “plug,” which blocks transport into or out of the ER. The plug is displaced when the hydrophobic region of a nascent polypeptide interacts with the “seam” region of Sec61, allowing translocation of the polypeptide into the ER lumen. In mammals, only short proteins (<160 amino acids) can enter the ER posttranslationally, and proteins smaller than 120 amino acids are obliged to use this pathway. Some of the translocation competence is maintained by the binding of calmodulin to the signal sequence. Upon arrival at the Sec61 channel, the signal peptide or signal anchor intercalates between transmembrane domains (“TMDs”) 2 and 7 of Sec61α, which form the lateral portion of the gate, allowing the channel to open for soluble secretory proteins. As the Sec61 channel consists of 10 TMDs (Sec61α) surrounded by a hydrophobic clamp formed by Sec61γ, channel opening is dependent on conformational changes that involve practically all TMDs.

Inhibition of protein transport across the ER membrane has the potential to treat or prevent diseases, such as the growth of cancer cells and inflammation. Known secretion inhibitors, which range from broad-spectrum to highly substrate-specific, can interfere with virtually any stage of this multistep process, and even with transport of endocytosed antigens into the cytosol for cross-presentation. These inhibitors interact with the signal peptide, chaperones, or the Sec61 channel to block substrate binding or to prevent the conformational changes needed for protein import into the ER. Examples of protein secretion inhibitors include, calmodulin inhibitors (e.g., E6 Berbamine and Ophiobolin A), Lanthanum, sterols, cyclodepsipeptides (e.g., HUN-7293, CAM741, NF1028, Cotrainsin, Apratoxin A, Decatransin, Valinomycin), CADA, Mycolactone, Eeyarestatin I (“ESI”), and Exotoxin A. However, the above secretion inhibitors suffer from one or more of the following: lack selectivity for the Sec61 channel, challenging manufacture due to structural complexity, and molecular weight limited administration, bio-availability and distribution.

Thus, a need exits for new inhibitors of protein secretion.

### SUMMARY

Provided herein are compounds having a structure of any one of formula (I), (I′), (II), (III), or (IV), or as listed in Table E below:

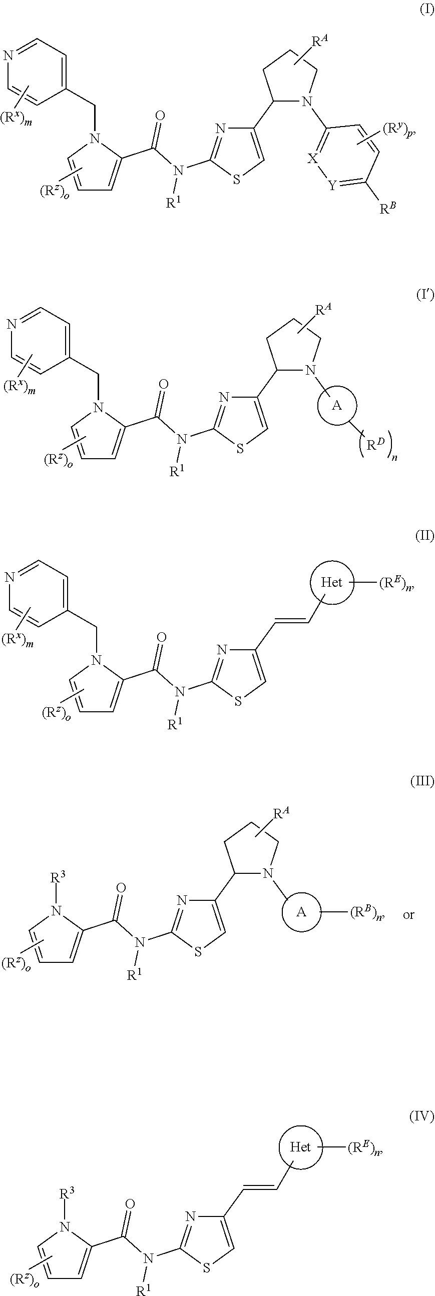

where the substituents are as disclosed below.

Also provided are pharmaceutical compositions comprising the compound or salt described herein and a pharmaceutically acceptable carrier.

Further provided are methods of inhibiting protein secretion in a cell comprising contacting the cell with the compound, salt, or pharmaceutical composition described herein in an amount effective to inhibit secretion.

In some embodiments, the protein is a checkpoint protein. In some embodiments, the protein is a cell-surface protein, endoplasmic reticulum associated protein, or secreted protein involved in regulation of anti-tumor immune response. In various cases, the protein is at least one of PD-1, PD-L1, TIM-1, LAG-3, CTLA4, BTLA, OX-40, B7H1, B7H4, CD137, CD47, CD96, CD73, CD40, VISTA, TIGIT, LAIR1, CD160, 2B4, TGFRβ and combinations thereof. In some cases, the protein is selected from the group consisting of HER3, TNFα, IL2, and PD1. In some embodiments, the contacting comprises administering the compound or the composition to a subject in need thereof.

The disclosure also provides methods for treating inflammation in a subject comprising administering to the subject a therapeutically effective amount of the compound, salt, or pharmaceutical composition described herein.

The disclosure further provides methods for treating cancer in a subject comprising administering to the subject a therapeutically effective amount of the compound, salt, or pharmaceutical composition described herein. In some embodiments, the cancer is melanoma, multiple myeloma, prostate cancer, lung cancer, pancreatic cancer, squamous cell carcinoma, leukemia, lymphoma, a neuroendocrine tumor, bladder cancer, or colorectal cancer. In some cases, the cancer is selected from the group consisting of prostate, lung, bladder, colorectal, and multiple myeloma. In some cases, the cancer is non-small cell lung carcinoma, squamous cell carcinoma, leukemia, acute myelogenous leukemia, chronic myelogenous leukemia, lymphoma, NPM/ALK-transformed anaplastic large cell lymphoma, diffuse large B cell lymphoma, neuroendocrine tumors, breast cancer, mantle cell lymphoma, renal cell carcinoma, rhabdomyosarcoma, ovarian cancer, endometrial cancer, small cell carcinoma, adenocarcinoma, gastric carcinoma, hepatocellular carcinoma, pancreatic cancer, thyroid carcinoma, anaplastic large cell lymphoma, hemangioma, or head and neck cancer. In various cases, the cancer is a solid tumor. In various cases, the cancer is head and neck cancer, squamous cell carcinoma, gastric carcinoma, or pancreatic cancer.

Further provided are methods for treating an autoimmune disease in a subject comprising administering to the subject a therapeutically effective amount of the compound, salt, or pharmaceutical composition described herein. In some embodiments, the autoimmune disease is psoriasis, dermatitis, systemic scleroderma, sclerosis, Crohn's disease, ulcerative colitis; respiratory distress syndrome, meningitis; encephalitis; uveitis; colitis; glomerulonephritis; eczema, asthma, chronic inflammation; atherosclerosis; leukocyte adhesion deficiency; rheumatoid arthritis; systemic lupus erythematosus (SLE); diabetes mellitus; multiple sclerosis; Reynaud's syndrome; autoimmune thyroiditis; allergic encephalomyelitis; Sjorgen's syndrome; juvenile onset diabetes; tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia; myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia.

The disclosure also provides methods for the treatment of an immune-related disease in a subject comprising administering to the subject a therapeutically effective amount of the compound, salt, or pharmaceutical composition described herein. In some embodiments, the immune-related disease is rheumatoid arthritis, lupus, inflammatory bowel disease, multiple sclerosis, or Crohn's disease.

Further provided are methods for treating neurodegenerative disease in a subject comprising administering to the subject a therapeutically effective amount of the compound, salt, or pharmaceutical composition described herein. In some cases, the neurodegenerative disease is multiple sclerosis.

Also provided are methods for treating an inflammatory disease in a subject comprising administering to the subject a therapeutically effective amount of the compound, salt, or pharmaceutical composition described herein. In some embodiments, the inflammatory disease is bronchitis, conjunctivitis, myocarditis, pancreatitis, chronic cholecstitis, bronchiectasis, aortic valve stenosis, restenosis, psoriasis or arthritis.

Further aspects and advantages will be apparent to those of ordinary skill in the art from a review of the following detailed description. The description hereafter includes specific embodiments with the understanding that the disclosure is illustrative, and is not intended to limit the disclosure to the specific embodiments described herein.

### DETAILED DESCRIPTION

Provided herein are compounds that inhibit protein secretion. The compounds described herein can be used to treat or prevent diseases associated with excessive protein secretion, such as inflammation and cancer, improving the quality of life for afflicted individuals.

### Compounds of Formula (I) or (I′)

Compounds, or salt thereof, disclosed herein can have a structure of formula (I) or (I′):

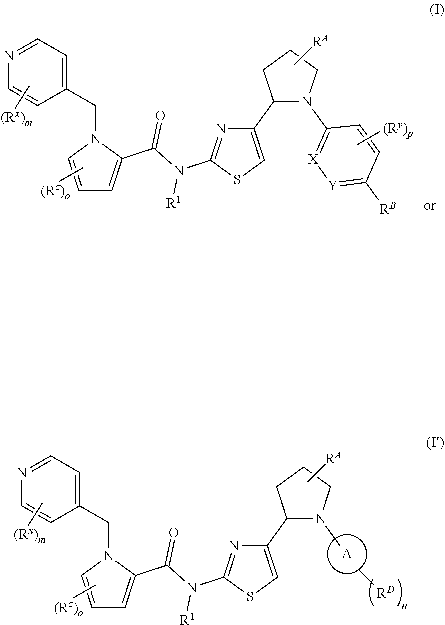

wherein
R¹ is H, C₁₋₃alkyl, or SO₂C₁₋₆alkyl;
each of X and Y is independently N or CR^{C};
ring A is a 6-membered heteroaryl having 2 nitrogen ring atoms;
R^{A} is H, C₁₋₆alkyl, OR^{N}, N(R^{N})₂, OC₁₋₆alkylene-N(R^{N})₂, or OC₁₋₆alkylene-OR^{N};
R^{B} is C₁₋₆alkyl, C₁₋₆alkoxy, C₁₋₃alkylene-C₁₋₃alkoxy, O—C₁₋₃alkylene-C₁₋₃alkoxy, C₁₋₆haloalkyl, C₁₋₆ hydroxyalkyl, O—C₁₋₆hydroxyalkyl, halo, C₀₋₃alkylene-CO₂R^{N}, C₀₋₃alkylene-N(R^{N})₂, OC₁₋₃alkylene-N(R^{N})₂, NO₂, C₀₋₃alkylene-C(O)N(R^{N})₂, C₀₋₃alkylene-N(R^{N})C(O)R^{N}, OC₁₋₃alkylene-N(R^{N})C(O)R^{N}, C₀₋₃alkylene-N(R^{N})C(O)N(R^{N})₂, C₀₋₃alkylene-N(R^{N})SO₂R^{N}, C₀₋₃alkylene-N(R^{N})C(O)OR^{N}, C₀₋₃alkylene-OC(O)N(R^{N})₂, C₀₋₃alkylene-Het, C₀₋₃alkylene-OHet, C₀₋₃alkylene-NHCO₂Het, C₀₋₃alkylene-OC(O)Het, C₀₋₃alkylene-N(R^{N})Het or C₀₋₃alkylene-N(R^{N})C(O)Het, or
if
   (1) m is 1 or 2;
   (2) at least one of X and Y is N,
   (3) at least one R^{C} is other than H, or
   (4) at least one of o and p is 1,
   then R^{B} can be H; or
if Y is CR^{C}, then R^{C} and R^{B} can combine to form a 6-membered fused ring with the carbons to which they are attached having 0-2 ring heteroatoms selected from N, O, and S and optionally substituted with 1 or 2 substituents independently selected from oxo, halo, and C₁₋₆alkyl;
Het is an aromatic or non-aromatic 4-7 membered ring having 0-3 ring heteroatoms selected from N, O, and S, and Het is optionally substituted with 1 or 2 substituents independently selected from C₁₋₆alkyl, halo, OR^{N}, oxo, C(O)R^{N}, C(O)C₃₋₆cycloalkyl, C(O)N(R^{N})₂, SOR^{N}, SO₂R^{N}, and SO₂N(R^{N})₂;
each R^{C} is independently H, halo, C₁₋₆alkoxy, N(R^{N})₂, CN, Het, or C₁₋₆alkyl;
n is 0, 1, or 2;
each R^{D}, when present, is independently halo, C₁₋₆alkoxy, or C₁₋₆alkyl;
m is 0, 1, or 2;
each R^{x}, when present, is independently halo or C₁₋₆alkyl;
p is 0 or 1;
R^{y}, when present, is C₁₋₆alkyl or halo;
o is 0 or 1;
R^{z}, when present, is CN, halo, C(O)N(R^{N})₂, C₁₋₆alkyl, C₁₋₆alkoxy, C₁₋₆hydroxyalkyl, or C₁₋₆haloalkyl; and
each R^{N} is independently H, C₁₋₆alkyl, C₁₋₆hydroxyalkyl, or C₁₋₆haloalkyl,
   with the proviso that when each of m, p, and o is 0, R¹ is H, X and Y are each CR^{C}, and at least one R^{C} is F, then R^{B} is not F.

In various cases, R¹ is H. In various cases, R^{A} is H. In some cases, R^{A} is OC₁₋₆alkylene-N(R^{N})₂ or OC₁₋₆ alkylene-OR^{N}. In some cases, R^{A} is OR^{N} or N(R^{N})₂. In various cases, each R^{N} is H or methyl.

In various cases, X is N. In some cases, X is CR^{C}. In various cases, Y is N. In various cases, Y is CR^{C}. In various cases, X and Y are each CR^{C}. In various cases, at least one R^{C} is H. In various cases, each R^{C} is H. In various cases, at least one R^{C} is halo, and in some specific cases, the halo is fluoro. In various cases, at least one R^{C} is C₁₋₆alkoxy or C₁₋₆alkyl. In various cases, R^{C} and R^{B} combine to form a 6-membered fused ring with the carbons to which they are attached having 0-1 ring heteroatoms selected from N, O, and S and optionally substituted with 1 or 2 substituents independently selected from oxo, halo, and C₁₋₆alkyl. In various cases, at least one R^{C} is N(R^{N})₂, CN or Het.

In various cases, R^{B} is C₁₋₆alkyl, C₁₋₆alkoxy, C₁₋₃alkylene-C₁₋₃alkoxy, C₁₋₆haloalkyl, C₁₋₆hydroxyalkyl, halo, C₃₋₆cycloalkyl, CO₂R^{N}, C₀₋₃alkylene-N(R^{N})₂, NO₂, C₀₋₃alkylene-C(O)N(R^{N})₂, C₀₋₃alkylene-N(R^{N})C(O)R^{N}, Het, or OHet. In various cases, R^{B} is C₀₋₃alkylene-N(R^{N})C(O)R^{N}, OC₁₋₃alkylene-N(R^{N})C(O)R^{N}, C₀₋₃alkylene-N(R^{N})C(O)N(R^{N})₂, C₀₋₃alkylene-N(R^{N})C(O)OR^{N}, or C₁₋₆haloalkyl. In various cases, R^{B} is C₁₋₆alkyl. In various cases, R^{B} is is C₁₋₆alkyl, C₁₋₆haloalkyl, C₁₋₆hydroxyalkyl, or halo. In various cases, R^{B} is CO₂R^{N}, C₀₋₃alkylene-N(R^{N})₂, C₀₋₃alkylene-C(O)N(R^{N})₂, or C₀₋₃alkylene-N(R^{N})C(O)R^{N}. In various cases, each R^{N} is H or methyl. In various cases, R^{B} is O—C₁₋₃alkylene-C₁₋₃alkoxy, O—C₁₋₆hydroxyalkyl, NHC(O)C₃₋₆cycloalkyl with the cycloalkyl optionally substituted with OH, OC₁₋₃alkylene-N(R^{N})₂, OC₁₋₃alkylene-N(R^{N})C(O)R^{N}, C₀₋₃alkylene-N(R^{N})C(O)N(R^{N})₂, C₀₋₃alkylene-N(R^{N})SO₂R^{N}, C₀₋₃alkylene-N(R^{N})C(O)OR^{N}, C₁₋₃alkylene-Het, N(R^{N})Het, or N(R^{N})C(O)OHet.

In various cases, R^{B} is C₃₋₆cycloalkyl, Het, or OHet. In some cases, Het is imidazole or oxazole. In some cases, Het is a non-aromatic 4-7 membered heterocycle having 1-3 ring heteroatoms. In some cases, Het is tetrahydropyran, piperidine, morpholine, tetrahydrofuran, pyrrolindine, or oxetanyl. In various cases, Het is unsubstituted. In some cases, Het is substituted, and in some specific cases is mono-substituted and in other specific cases is di-substituted. In some cases, Het is a non-aromatic 4-7 membered heterocycle and is substituted with oxo. In some cases, Het is substituted with C₁₋₆alkyl. In some cases, Het is substituted with C₁₋₆alkoxy. In some cases, Het is substituted with C(O)R^{N} or SO₂R^{N}. In some cases, Het is substituted with halo. In some case, C(O)N(R^{N})₂.

In various cases, R^{B} is H, with the proviso that at least one of: (1) m is 1 or 2; (2) at least one of X and Y is N, (3) at least one R^{C} is other than H, and (4) at least one of o and p is 1. In some cases, Y is CR^{C}, then R^{C} and R^{B} can combine to form a 6-membered fused ring with the carbons to which they are attached having 0-1 ring heteroatoms selected from N, O, and S and optionally substituted with 1 or 2 substituents independently selected from oxo, halo, and C₁₋₆alkyl.

In some cases, m is 0. In various cases, m is 1, and in some specific cases, R^{x} is at 2-position of pyridine, i.e.,

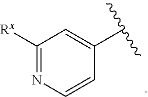

In some cases, m is 2, and in some specific cases, one R^{x} is at 2-position and other R^{x} is at 6-position of pyridine, i.e.,

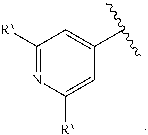

In various cases, R^{x} is halo or methyl. In some cases, at least one R^{x} is fluoro. In some cases, when m is 2, each R^{x} is fluoro.

In various cases, o is 0. In some cases, o is 1, and in some specific cases, R^{z} is meta to the ring nitrogen, i.e.,

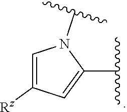

In various cases, p is 0. In some cases, p is 1. In cases where p is 1, R^{y} can be methyl or halo (e.g., fluoro).

In some cases, the compound of formula (I) has a structure of:

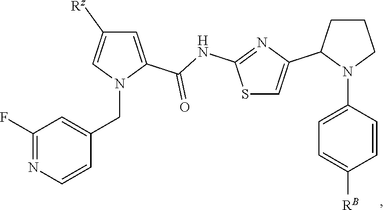

where R^{z} and R^{B} are as described herein.

In various cases, each R^{N} is H or methyl. In some cases, at least one R^{N} is C₁₋₆hydroxyalkyl or C₁₋₆haloalkyl.

In various cases, the compound has a structure of Formula (I′). In some cases, ring A is pyrimidinyl. In some cases, ring A is pyrazinyl. In various cases, ring A is pyradazinyl.

In various cases, n is 0. In some cases, n is 1. In some cases, n is 2. In some cases where n is 1 or 2, at least one R^{D} is halo, and more specifically, is fluoro. In some cases where n is 1 or 2, at least one R^{D} is C₁₋₆alkoxy. In some cases where n is 1 or 2, at least one R^{D} is C₁₋₆alkyl.

In various cases, the compound of Formula (I) or (I′) is a structure as shown in Table A, or a pharmaceutically acceptable salt thereof:

TABLE A

| | |
|---|---|
| A1 | 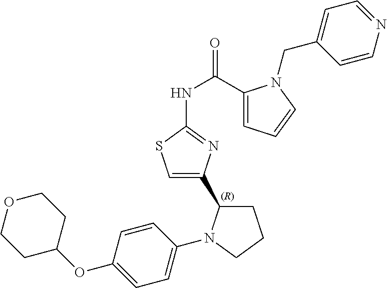 |
| A2 | 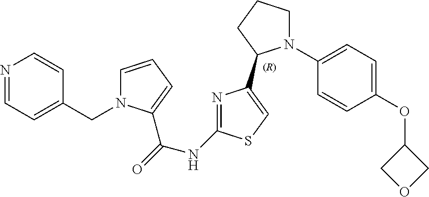 |
| A3 | 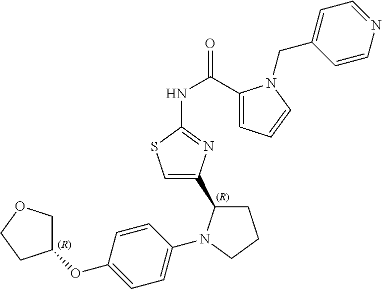 |
| A4 | 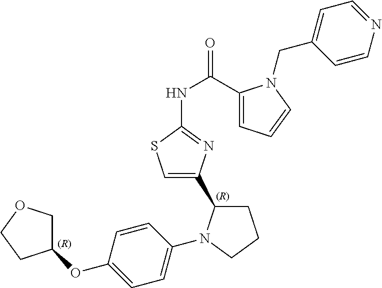 |
| A5 | 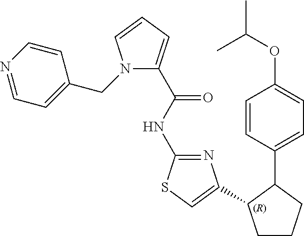 |
| A6 | 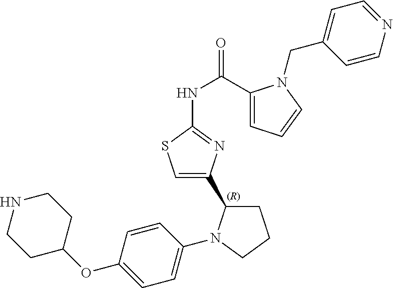 |
| A7 | 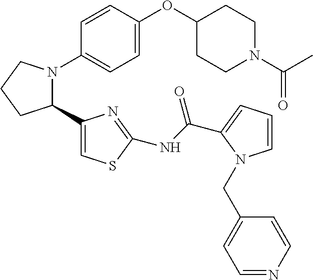 [SMILES: CC(=O)N1CCC(Oc2ccc(N3CCC[C@@H]3c3csc(NC(=O)c4cccn4Cc4ccncc4)n3)cc2)CC1] |
| A8 | 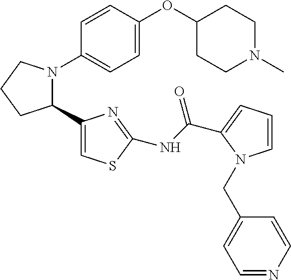 [SMILES: CN1CCC(Oc2ccc(N3CCC[C@@H]3c3csc(NC(=O)c4cccn4Cc4ccncc4)n3)cc2)CC1] |
| A9 | 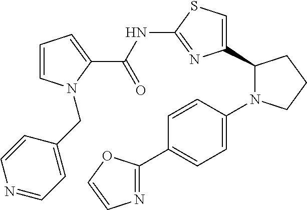 [SMILES: O=C(Nc1nc([C@H]2CCCN2c2ccc(-c3ncco3)cc2)cs1)c1cccn1Cc1ccncc1] |
| A10 | 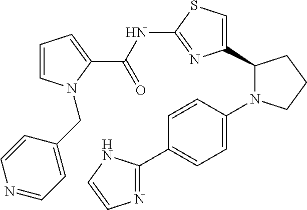 [SMILES: O=C(Nc1nc([C@H]2CCCN2c2ccc(-c3ncc[nH]3)cc2)cs1)c1cccn1Cc1ccncc1] |
| A11 | 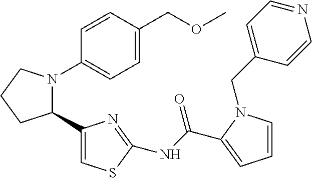 [SMILES: COCc1ccc(N2CCC[C@@H]2c2csc(NC(=O)c3cccn3Cc3ccncc3)n2)cc1] |
| A12 | 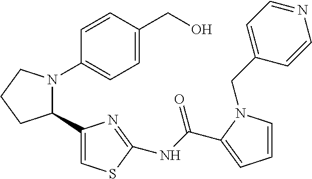 [SMILES: O=C(Nc1nc([C@H]2CCCN2c2ccc(CO)cc2)cs1)c1cccn1Cc1ccncc1] |
| A13 | 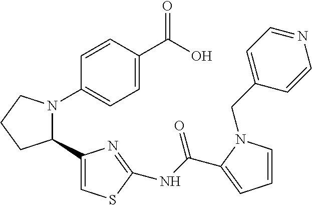 [SMILES: O=C(O)c1ccc(N2CCC[C@@H]2c2csc(NC(=O)c3cccn3Cc3ccncc3)n2)cc1] |
| A14 | 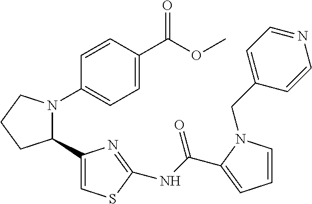 [SMILES: COC(=O)c1ccc(N2CCC[C@@H]2c2csc(NC(=O)c3cccn3Cc3ccncc3)n2)cc1] |
| A15 | 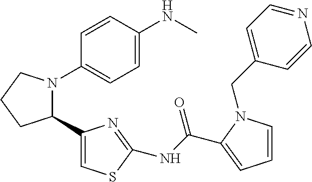 [SMILES: CNc1ccc(N2CCC[C@@H]2c2csc(NC(=O)c3cccn3Cc3ccncc3)n2)cc1] |
| A16 | 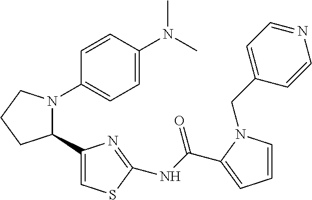 [SMILES: CN(C)c1ccc(N2CCC[C@@H]2c2csc(NC(=O)c3cccn3Cc3ccncc3)n2)cc1] |
| A17 | 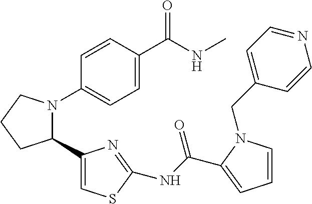 [SMILES: CNC(=O)c1ccc(N2CCC[C@@H]2c2csc(NC(=O)c3cccn3Cc3ccncc3)n2)cc1] |
| A18 | 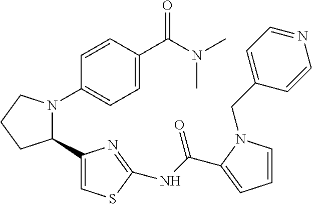 [SMILES: CN(C)C(=O)c1ccc(N2CCC[C@@H]2c2csc(NC(=O)c3cccn3Cc3ccncc3)n2)cc1] |
| A19 | 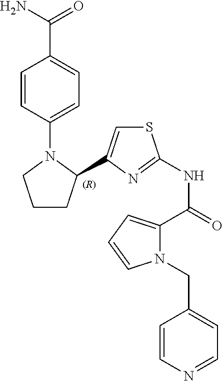 |
| A20 | 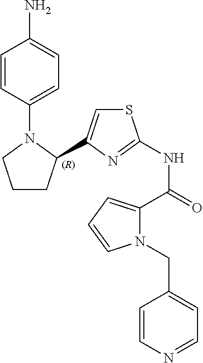 |
| A21 | 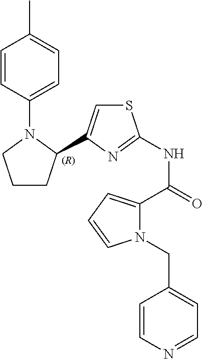 |
| A22 | 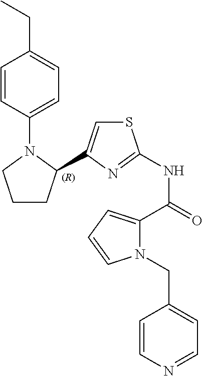 |
| A23 | 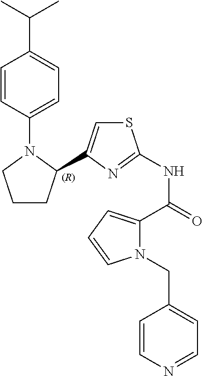 |
| A24 | 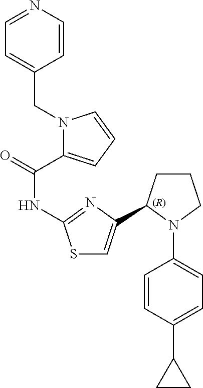 |
| A25 | 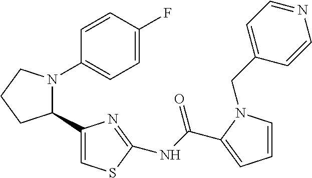 [SMILES: O=C(Nc1nc([C@H]2CCCN2c2ccc(F)cc2)cs1)c1cccn1Cc1ccncc1] |
| A26 | 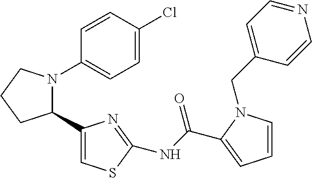 [SMILES: O=C(Nc1nc([C@H]2CCCN2c2ccc(Cl)cc2)cs1)c1cccn1Cc1ccncc1] |
| A27 | 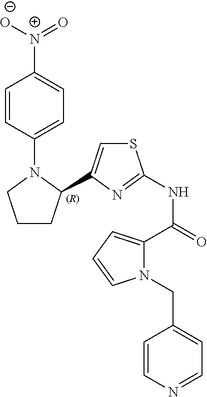 |
| A28 | 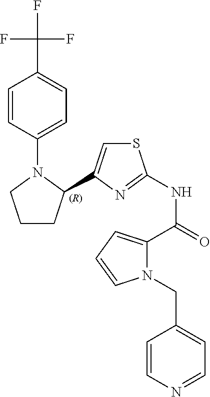 |
| A29 | 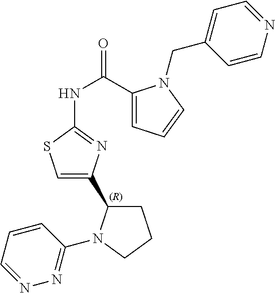 |
| A30 | 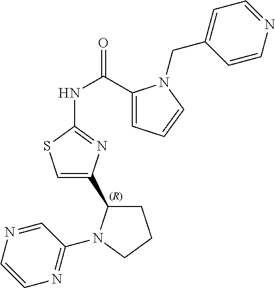 |
| A31 | 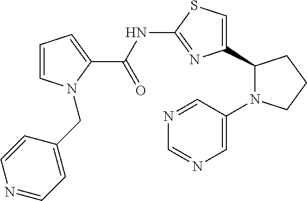 [SMILES: O=C(Nc1nc([C@H]2CCCN2c2cncnc2)cs1)c1cccn1Cc1ccncc1] |
| A32 | 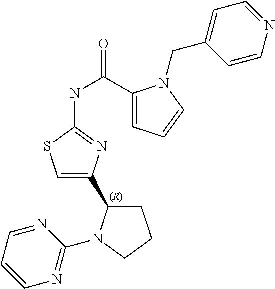 |
| A33 | 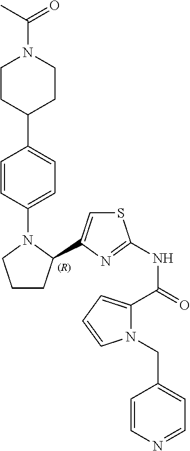 |
| A34 | 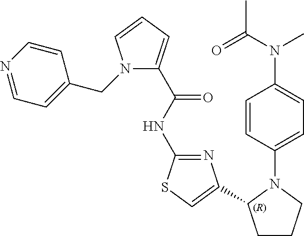 |
| A35 | 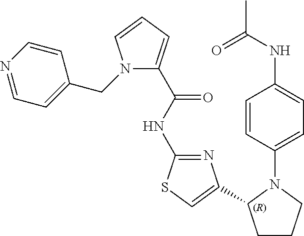 |
| A36 | 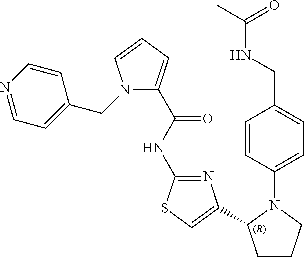 |
| A37 | 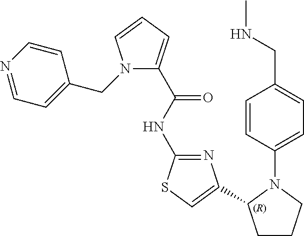 |
| A38 | 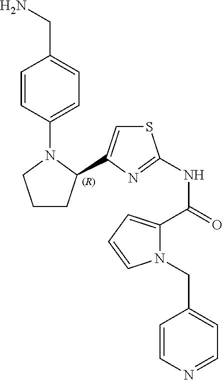 |
| A39 | 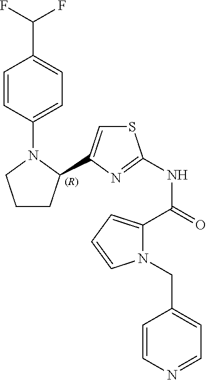 |
| A40 | 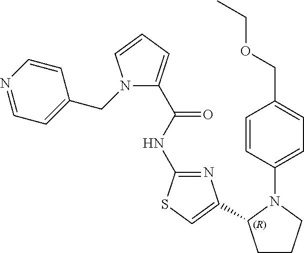 |
| A41 | 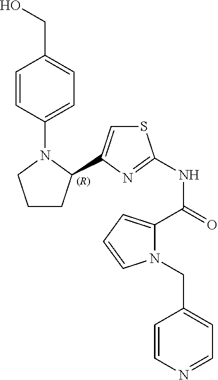 |
| A44 | 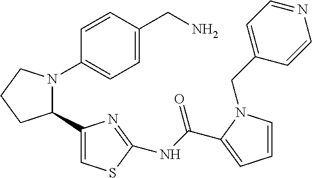 [SMILES: NCc1ccc(N2CCC[C@@H]2c2csc(NC(=O)c3cccn3Cc3ccncc3)n2)cc1] |
| A45 | 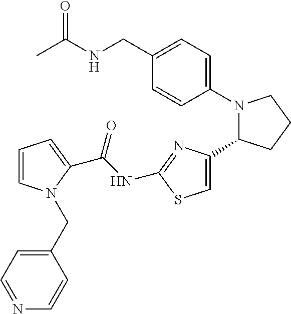 [SMILES: CC(=O)NCc1ccc(N2CCC[C@@H]2c2csc(NC(=O)c3cccn3Cc3ccncc3)n2)cc1] |
| A46 | 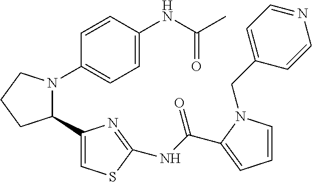 [SMILES: CC(=O)Nc1ccc(N2CCC[C@@H]2c2csc(NC(=O)c3cccn3Cc3ccncc3)n2)cc1] |
| A47 | 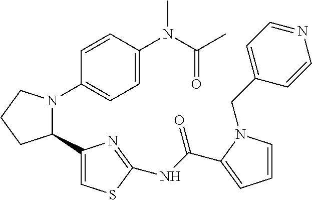 [SMILES: CC(=O)N(C)c1ccc(N2CCC[C@@H]2c2csc(NC(=O)c3cccn3Cc3ccncc3)n2)cc1] |
| A48 | 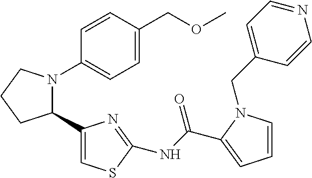 [SMILES: COCc1ccc(N2CCC[C@@H]2c2csc(NC(=O)c3cccn3Cc3ccncc3)n2)cc1] |
| A49 | 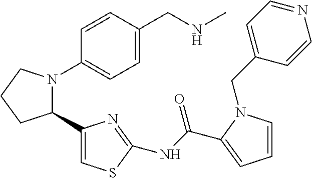 [SMILES: CNCc1ccc(N2CCC[C@@H]2c2csc(NC(=O)c3cccn3Cc3ccncc3)n2)cc1] |
| A51 | 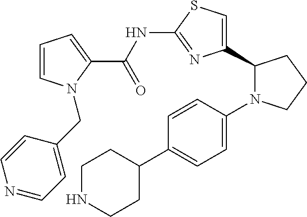 [SMILES: O=C(Nc1nc([C@H]2CCCN2c2ccc(C3CCNCC3)cc2)cs1)c1cccn1Cc1ccncc1] |
| A52 | 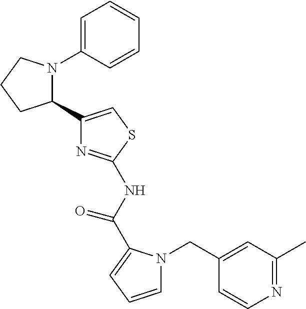 [SMILES: Cc1cc(Cn2cccc2C(=O)Nc2nc([C@H]3CCCN3c3ccccc3)cs2)ccn1] |
| A53 | 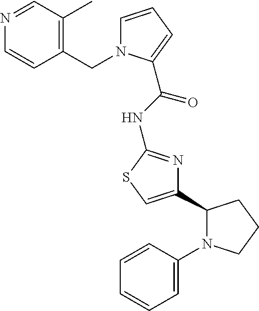 [SMILES: Cc1cnccc1Cn1cccc1C(=O)Nc1nc([C@H]2CCCN2c2ccccc2)cs1] |
| A55 | 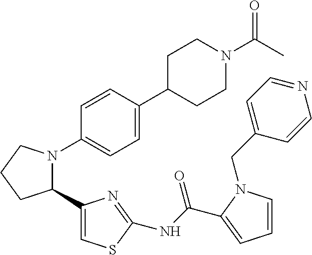 [SMILES: CC(=O)N1CCC(c2ccc(N3CCC[C@@H]3c3csc(NC(=O)c4cccn4Cc4ccncc4)n3)cc2)CC1] |
| A56 | 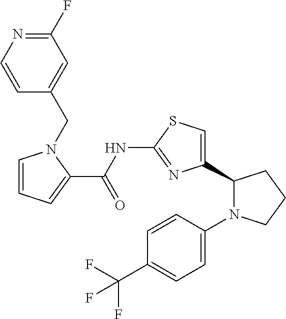 [SMILES: O=C(Nc1nc([C@H]2CCCN2c2ccc(C(F)(F)F)cc2)cs1)c1cccn1Cc1ccnc(F)c1] |
| A57 | 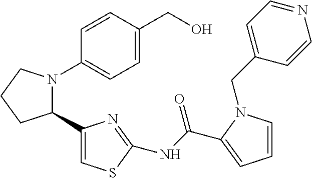 [SMILES: O=C(Nc1nc([C@H]2CCCN2c2ccc(CO)cc2)cs1)c1cccn1Cc1ccncc1] |
| A58 | 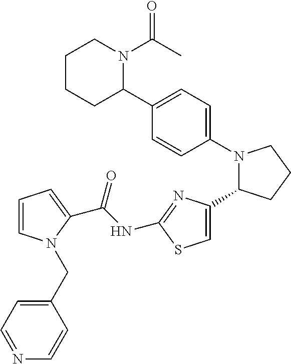 [SMILES: CC(=O)N1CCCCC1c1ccc(N2CCC[C@@H]2c2csc(NC(=O)c3cccn3Cc3ccncc3)n2)cc1] |
| A59 | 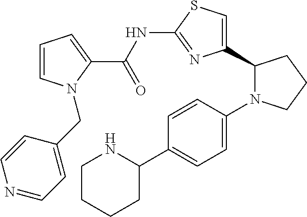 [SMILES: O=C(Nc1nc([C@H]2CCCN2c2ccc(C3CCCCN3)cc2)cs1)c1cccn1Cc1ccncc1] |
| A60 | 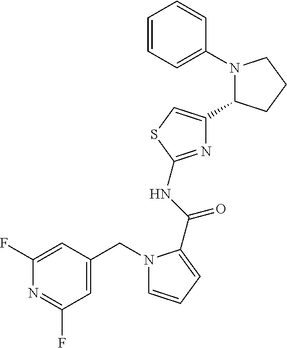 [SMILES: O=C(Nc1nc([C@H]2CCCN2c2ccccc2)cs1)c1cccn1Cc1cc(F)nc(F)c1] |
| A61 | 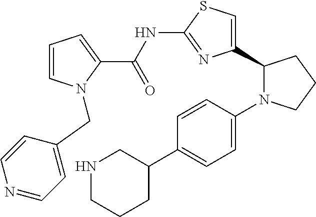 [SMILES: O=C(Nc1nc([C@H]2CCCN2c2ccc(C3CCCNC3)cc2)cs1)c1cccn1Cc1ccncc1] |
| A62 | 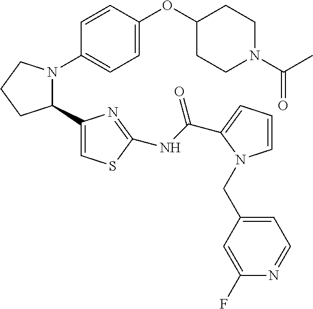 [SMILES: CC(=O)N1CCC(Oc2ccc(N3CCC[C@@H]3c3csc(NC(=O)c4cccn4Cc4ccnc(F)c4)n3)cc2)CC1] |
| A63 | 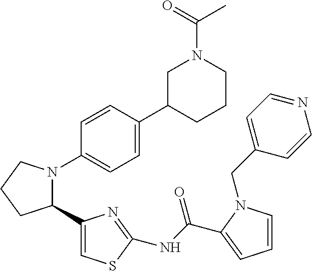 [SMILES: CC(=O)N1CCCC(c2ccc(N3CCC[C@@H]3c3csc(NC(=O)c4cccn4Cc4ccncc4)n3)cc2)C1] |
| A64 | 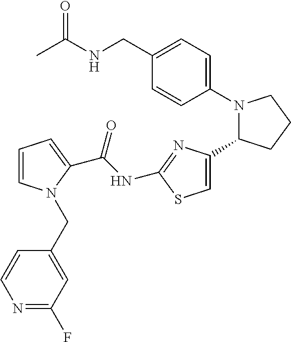 [SMILES: CC(=O)NCc1ccc(N2CCC[C@@H]2c2csc(NC(=O)c3cccn3Cc3ccnc(F)c3)n2)cc1] |
| A65 | 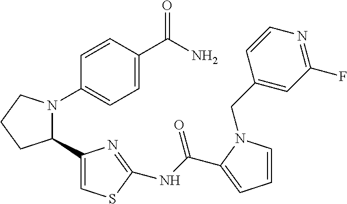 [SMILES: NC(=O)c1ccc(N2CCC[C@@H]2c2csc(NC(=O)c3cccn3Cc3ccnc(F)c3)n2)cc1] |
| A66 | 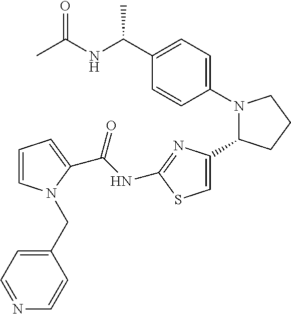 [SMILES: CC(=O)N[C@H](C)c1ccc(N2CCC[C@@H]2c2csc(NC(=O)c3cccn3Cc3ccncc3)n2)cc1] |
| A67 | 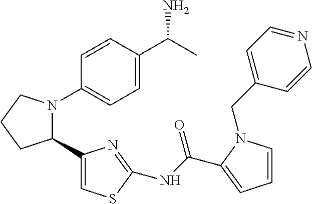 [SMILES: C[C@@H](N)c1ccc(N2CCC[C@@H]2c2csc(NC(=O)c3cccn3Cc3ccncc3)n2)cc1] |
| A68 | 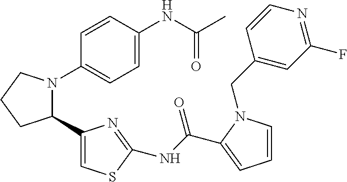 [SMILES: CC(=O)Nc1ccc(N2CCC[C@@H]2c2csc(NC(=O)c3cccn3Cc3ccnc(F)c3)n2)cc1] |
| A69 | 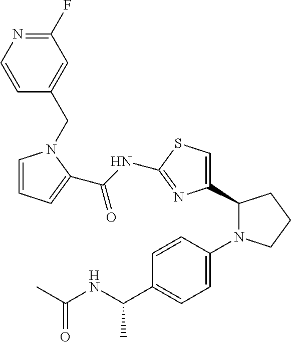 [SMILES: CC(=O)N[C@@H](C)c1ccc(N2CCC[C@@H]2c2csc(NC(=O)c3cccn3Cc3ccnc(F)c3)n2)cc1] |
| A70 | 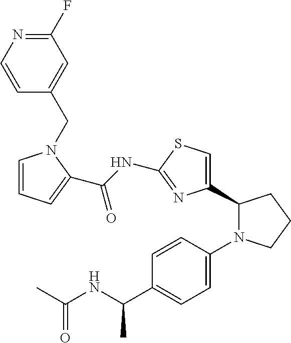 [SMILES: CC(=O)N[C@H](C)c1ccc(N2CCC[C@@H]2c2csc(NC(=O)c3cccn3Cc3ccnc(F)c3)n2)cc1] |
| A71 | 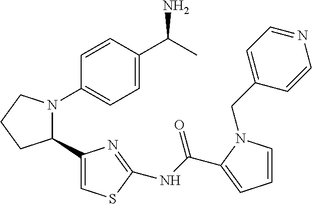 [SMILES: C[C@H](N)c1ccc(N2CCC[C@@H]2c2csc(NC(=O)c3cccn3Cc3ccncc3)n2)cc1] |
| A72 | 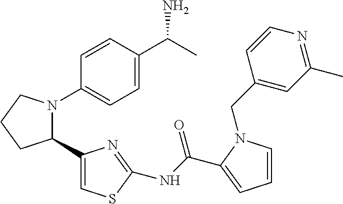 [SMILES: Cc1cc(Cn2cccc2C(=O)Nc2nc([C@H]3CCCN3c3ccc([C@@H](C)N)cc3)cs2)ccn1] |
| A73 | 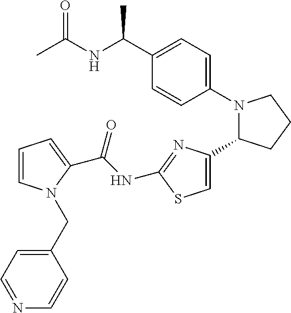 [SMILES: CC(=O)N[C@@H](C)c1ccc(N2CCC[C@@H]2c2csc(NC(=O)c3cccn3Cc3ccncc3)n2)cc1] |
| A74 | 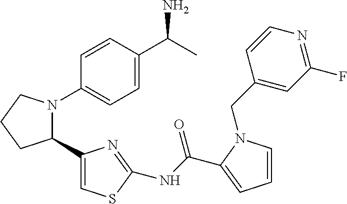 [SMILES: C[C@H](N)c1ccc(N2CCC[C@@H]2c2csc(NC(=O)c3cccn3Cc3ccnc(F)c3)n2)cc1] |
| A75 | 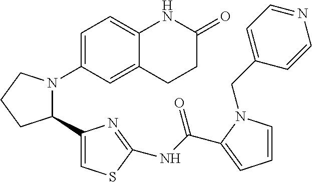 [SMILES: O=C1CCc2cc(N3CCC[C@@H]3c3csc(NC(=O)c4cccn4Cc4ccncc4)n3)ccc2N1] |
| A76 | 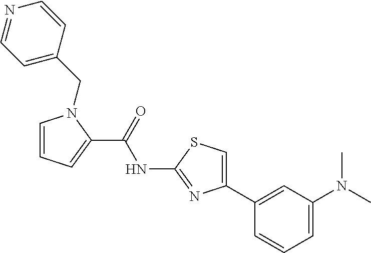 [SMILES: CN(C)c1cccc(-c2csc(NC(=O)c3cccn3Cc3ccncc3)n2)c1] |
| A77 | 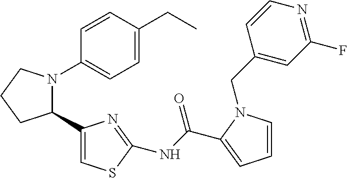 [SMILES: CCc1ccc(N2CCC[C@@H]2c2csc(NC(=O)c3cccn3Cc3ccnc(F)c3)n2)cc1] |
| A78 | 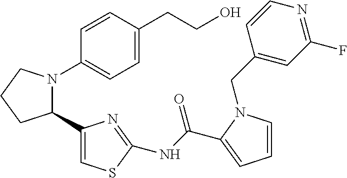 [SMILES: O=C(Nc1nc([C@H]2CCCN2c2ccc(CCO)cc2)cs1)c1cccn1Cc1ccnc(F)c1] |
| A79 | 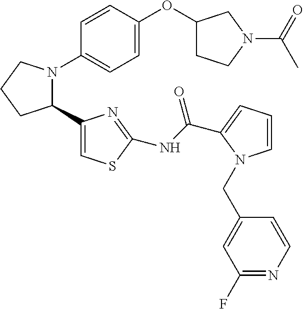 [SMILES: CC(=O)N1CCC(Oc2ccc(N3CCC[C@@H]3c3csc(NC(=O)c4cccn4Cc4ccnc(F)c4)n3)cc2)C1] |
| A80 | 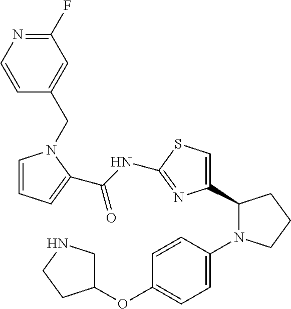 [SMILES: O=C(Nc1nc([C@H]2CCCN2c2ccc(OC3CCNC3)cc2)cs1)c1cccn1Cc1ccnc(F)c1] |
| A81 | 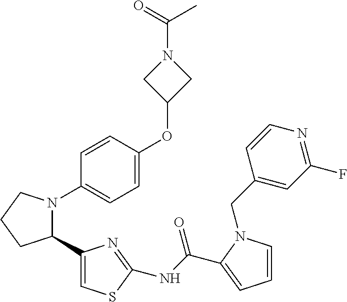 [SMILES: CC(=O)N1CC(Oc2ccc(N3CCC[C@@H]3c3csc(NC(=O)c4cccn4Cc4ccnc(F)c4)n3)cc2)C1] |
| A82 | 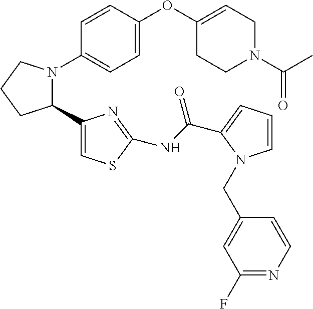 [SMILES: CC(=O)N1CC=C(Oc2ccc(N3CCC[C@@H]3c3csc(NC(=O)c4cccn4Cc4ccnc(F)c4)n3)cc2)CC1] |
| A83 | 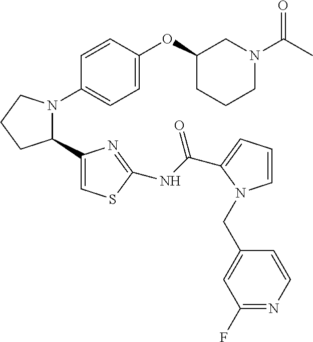 [SMILES: CC(=O)N1CCC[C@@H](Oc2ccc(N3CCC[C@@H]3c3csc(NC(=O)c4cccn4Cc4ccnc(F)c4)n3)cc2)C1] |
| A84 | 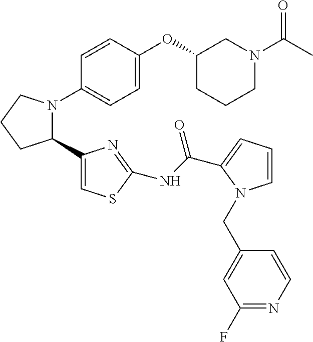 [SMILES: CC(=O)N1CCC[C@H](Oc2ccc(N3CCC[C@@H]3c3csc(NC(=O)c4cccn4Cc4ccnc(F)c4)n3)cc2)C1] |
| A85 | 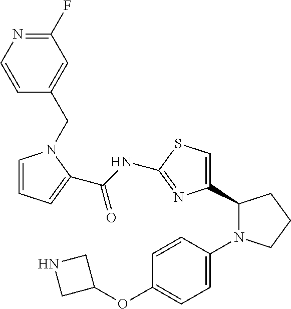 [SMILES: O=C(Nc1nc([C@H]2CCCN2c2ccc(OC3CNC3)cc2)cs1)c1cccn1Cc1ccnc(F)c1] |
| A87 | 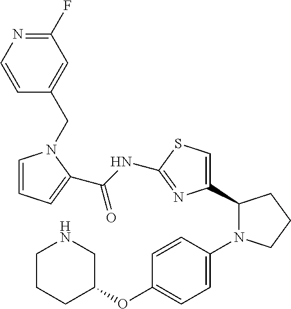 [SMILES: O=C(Nc1nc([C@H]2CCCN2c2ccc(O[C@@H]3CCCNC3)cc2)cs1)c1cccn1Cc1ccnc(F)c1] |
| A88 | 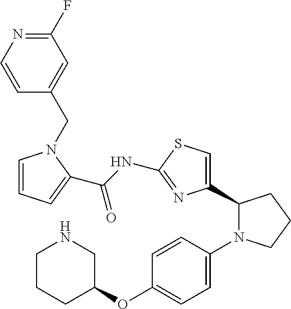 [SMILES: O=C(Nc1nc([C@H]2CCCN2c2ccc(O[C@H]3CCCNC3)cc2)cs1)c1cccn1Cc1ccnc(F)c1] |
| A89 | 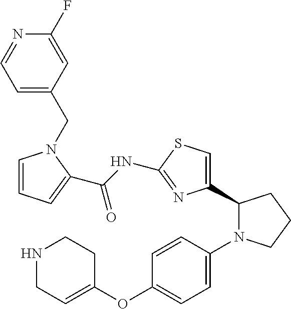 [SMILES: O=C(Nc1nc([C@H]2CCCN2c2ccc(OC3=CCNCC3)cc2)cs1)c1cccn1Cc1ccnc(F)c1] |
| A90 | 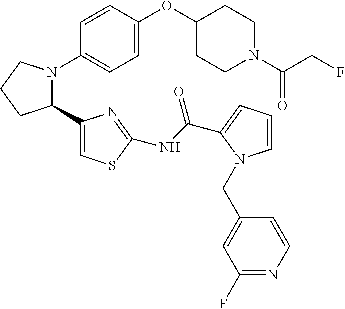 [SMILES: O=C(Nc1nc([C@H]2CCCN2c2ccc(OC3CCN(C(=O)CF)CC3)cc2)cs1)c1cccn1Cc1ccnc(F)c1] |
| A91 | 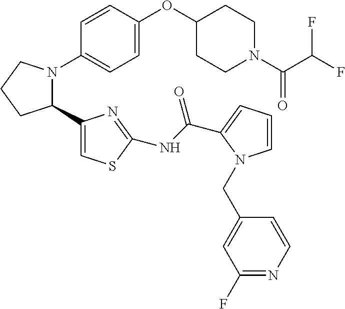 [SMILES: O=C(Nc1nc([C@H]2CCCN2c2ccc(OC3CCN(C(=O)C(F)F)CC3)cc2)cs1)c1cccn1Cc1ccnc(F)c1] |
| A92 | 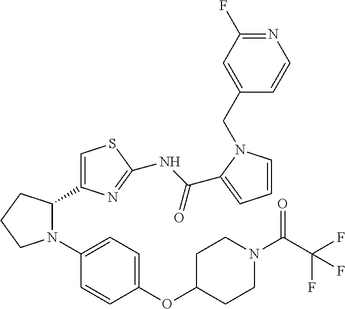 [SMILES: O=C(Nc1nc([C@H]2CCCN2c2ccc(OC3CCN(C(=O)C(F)(F)F)CC3)cc2)cs1)c1cccn1Cc1ccnc(F)c1] |
| A93 | 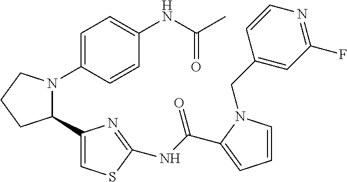 [SMILES: CC(=O)Nc1ccc(N2CCC[C@@H]2c2csc(NC(=O)c3cccn3Cc3ccnc(F)c3)n2)cc1] |
| A94 | 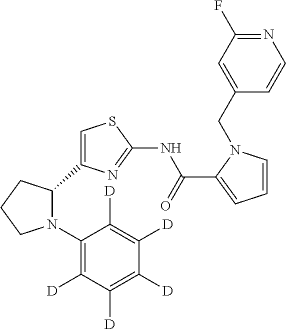 [SMILES: [2H]c1c([2H])c([2H])c(N2CCC[C@@H]2c2csc(NC(=O)c3cccn3Cc3ccnc(F)c3)n2)c([2H])c1[2H]] |
| A95 | 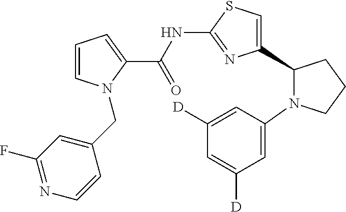 [SMILES: [2H]c1cc([2H])cc(N2CCC[C@@H]2c2csc(NC(=O)c3cccn3Cc3ccnc(F)c3)n2)c1] |
| A96 | 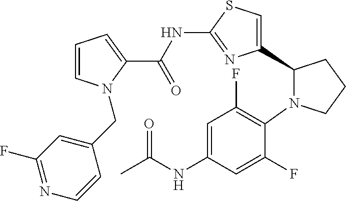 [SMILES: CC(=O)Nc1cc(F)c(N2CCC[C@@H]2c2csc(NC(=O)c3cccn3Cc3ccnc(F)c3)n2)c(F)c1] |
| A97 | 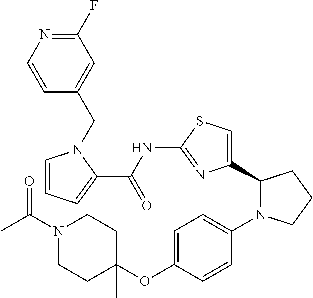 [SMILES: CC(=O)N1CCC(C)(Oc2ccc(N3CCC[C@@H]3c3csc(NC(=O)c4cccn4Cc4ccnc(F)c4)n3)cc2)CC1] |
| A98 | 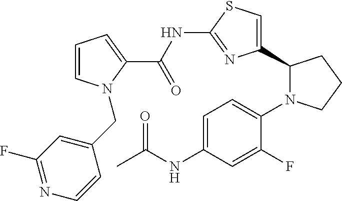 [SMILES: CC(=O)Nc1ccc(N2CCC[C@@H]2c2csc(NC(=O)c3cccn3Cc3ccnc(F)c3)n2)c(F)c1] |
| A99 | 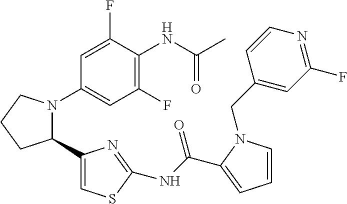 [SMILES: CC(=O)Nc1c(F)cc(N2CCC[C@@H]2c2csc(NC(=O)c3cccn3Cc3ccnc(F)c3)n2)cc1F] |
| A100 | 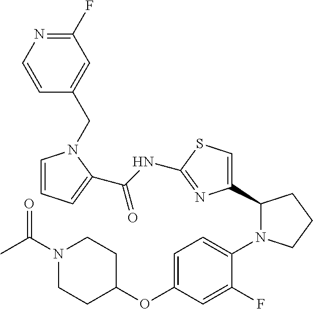 [SMILES: CC(=O)N1CCC(Oc2ccc(N3CCC[C@@H]3c3csc(NC(=O)c4cccn4Cc4ccnc(F)c4)n3)c(F)c2)CC1] |
| A101 | 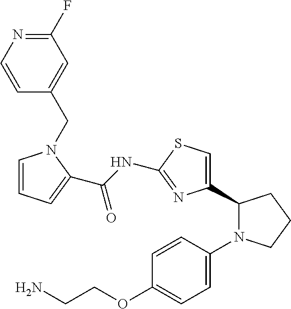 [SMILES: NCCOc1ccc(N2CCC[C@@H]2c2csc(NC(=O)c3cccn3Cc3ccnc(F)c3)n2)cc1] |
| A102 | 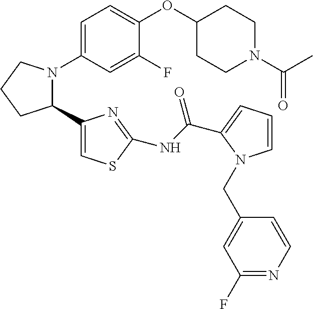 [SMILES: CC(=O)N1CCC(Oc2ccc(N3CCC[C@@H]3c3csc(NC(=O)c4cccn4Cc4ccnc(F)c4)n3)cc2F)CC1] |
| A103 | 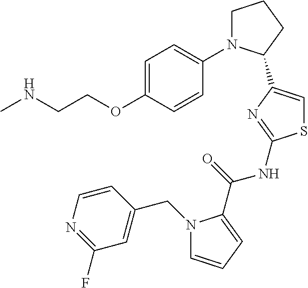 [SMILES: CNCCOc1ccc(N2CCC[C@@H]2c2csc(NC(=O)c3cccn3Cc3ccnc(F)c3)n2)cc1] |
| A104 | 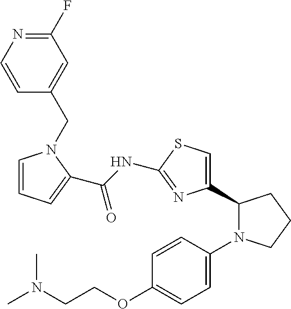 [SMILES: CN(C)CCOc1ccc(N2CCC[C@@H]2c2csc(NC(=O)c3cccn3Cc3ccnc(F)c3)n2)cc1] |
| A105 | 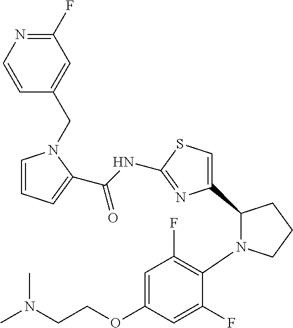 [SMILES: CN(C)CCOc1cc(F)c(N2CCC[C@@H]2c2csc(NC(=O)c3cccn3Cc3ccnc(F)c3)n2)c(F)c1] |
| A106 | 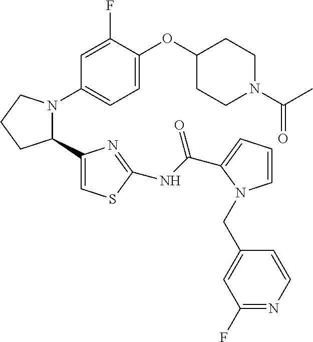 [SMILES: CC(=O)N1CCC(Oc2ccc(N3CCC[C@@H]3c3csc(NC(=O)c4cccn4Cc4ccnc(F)c4)n3)cc2F)CC1] |
| A107 | 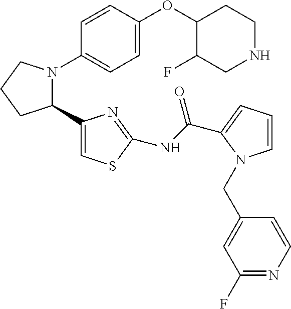 [SMILES: O=C(Nc1nc([C@H]2CCCN2c2ccc(OC3CCNCC3F)cc2)cs1)c1cccn1Cc1ccnc(F)c1] |
| A108 | 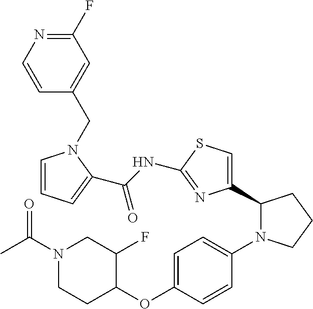 [SMILES: CC(=O)N1CCC(Oc2ccc(N3CCC[C@@H]3c3csc(NC(=O)c4cccn4Cc4ccnc(F)c4)n3)cc2)C(F)C1] |
| A109 | 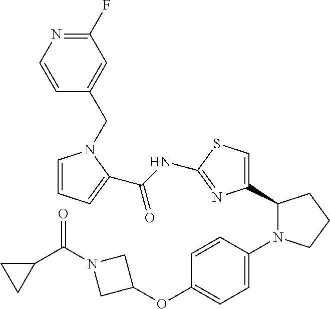 [SMILES: O=C(Nc1nc([C@H]2CCCN2c2ccc(OC3CN(C(=O)C4CC4)C3)cc2)cs1)c1cccn1Cc1ccnc(F)c1] |
| A110 | 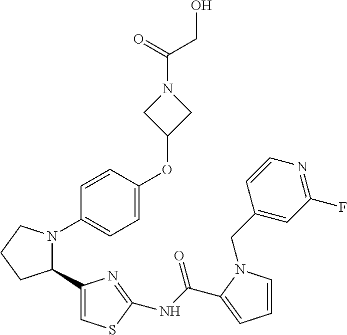 [SMILES: O=C(Nc1nc([C@H]2CCCN2c2ccc(OC3CN(C(=O)CO)C3)cc2)cs1)c1cccn1Cc1ccnc(F)c1] |
| A111 | 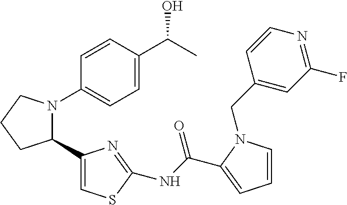 [SMILES: C[C@@H](O)c1ccc(N2CCC[C@@H]2c2csc(NC(=O)c3cccn3Cc3ccnc(F)c3)n2)cc1] |
| A112 | 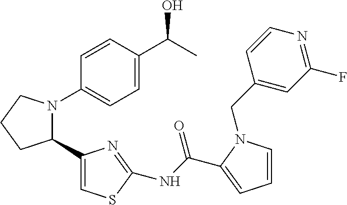 [SMILES: C[C@H](O)c1ccc(N2CCC[C@@H]2c2csc(NC(=O)c3cccn3Cc3ccnc(F)c3)n2)cc1] |
| A113 | 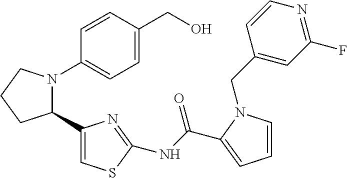 [SMILES: O=C(Nc1nc([C@H]2CCCN2c2ccc(CO)cc2)cs1)c1cccn1Cc1ccnc(F)c1] |
| A114 | 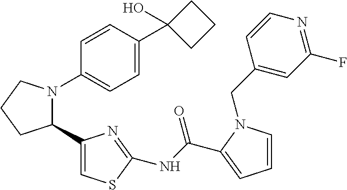 [SMILES: O=C(Nc1nc([C@H]2CCCN2c2ccc(C3(O)CCC3)cc2)cs1)c1cccn1Cc1ccnc(F)c1] |
| A115 | 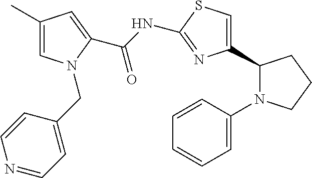 [SMILES: Cc1cc(C(=O)Nc2nc([C@H]3CCCN3c3ccccc3)cs2)n(Cc2ccncc2)c1] |
| A116 | 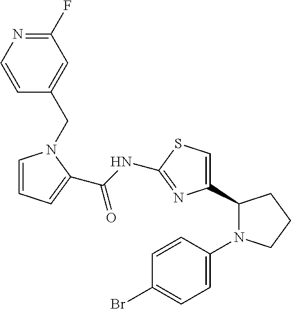 [SMILES: O=C(Nc1nc([C@H]2CCCN2c2ccc(Br)cc2)cs1)c1cccn1Cc1ccnc(F)c1] |
| A117 | 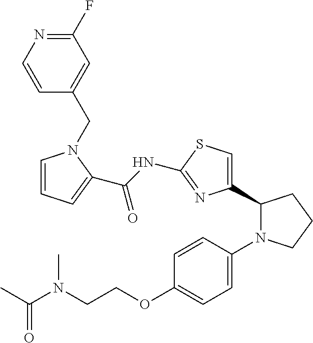 [SMILES: CC(=O)N(C)CCOc1ccc(N2CCC[C@@H]2c2csc(NC(=O)c3cccn3Cc3ccnc(F)c3)n2)cc1] |
| A118 | 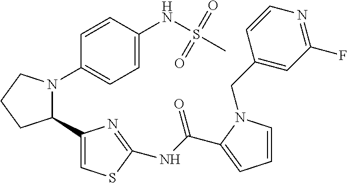 [SMILES: CS(=O)(=O)Nc1ccc(N2CCC[C@@H]2c2csc(NC(=O)c3cccn3Cc3ccnc(F)c3)n2)cc1] |
| A119 | 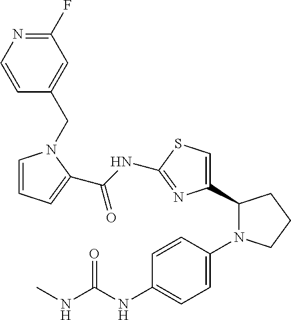 [SMILES: CNC(=O)Nc1ccc(N2CCC[C@@H]2c2csc(NC(=O)c3cccn3Cc3ccnc(F)c3)n2)cc1] |
| A120 | 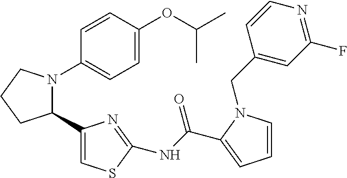 [SMILES: CC(C)Oc1ccc(N2CCC[C@@H]2c2csc(NC(=O)c3cccn3Cc3ccnc(F)c3)n2)cc1] |
| A121 | 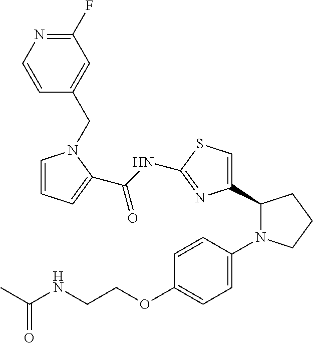 [SMILES: CC(=O)NCCOc1ccc(N2CCC[C@@H]2c2csc(NC(=O)c3cccn3Cc3ccnc(F)c3)n2)cc1] |
| A122 | 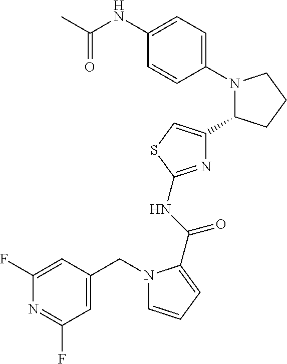 [SMILES: CC(=O)Nc1ccc(N2CCC[C@@H]2c2csc(NC(=O)c3cccn3Cc3cc(F)nc(F)c3)n2)cc1] |
| A123 | 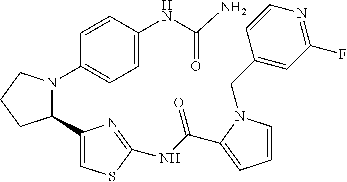 [SMILES: NC(=O)Nc1ccc(N2CCC[C@@H]2c2csc(NC(=O)c3cccn3Cc3ccnc(F)c3)n2)cc1] |
| A124 | 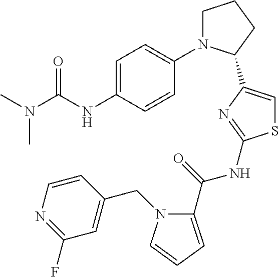 [SMILES: CN(C)C(=O)Nc1ccc(N2CCC[C@@H]2c2csc(NC(=O)c3cccn3Cc3ccnc(F)c3)n2)cc1] |
| A125 | 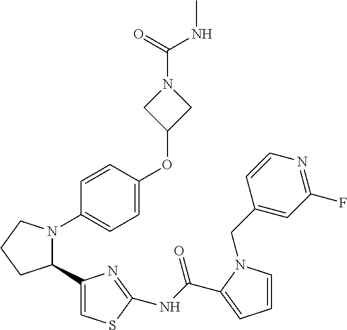 [SMILES: CNC(=O)N1CC(Oc2ccc(N3CCC[C@@H]3c3csc(NC(=O)c4cccn4Cc4ccnc(F)c4)n3)cc2)C1] |
| A126 | 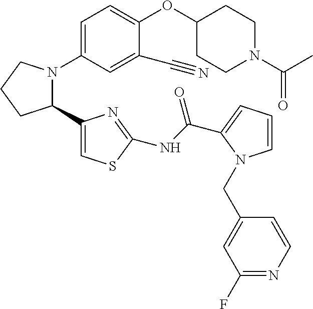 [SMILES: CC(=O)N1CCC(Oc2ccc(N3CCC[C@@H]3c3csc(NC(=O)c4cccn4Cc4ccnc(F)c4)n3)cc2C#N)CC1] |
| A127 | 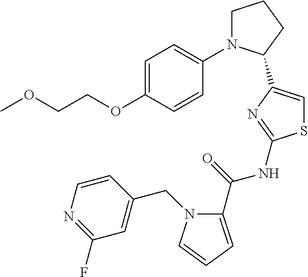 [SMILES: COCCOc1ccc(N2CCC[C@@H]2c2csc(NC(=O)c3cccn3Cc3ccnc(F)c3)n2)cc1] |
| A128 | 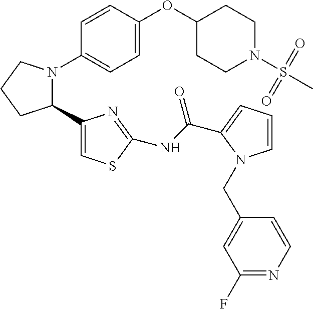 [SMILES: CS(=O)(=O)N1CCC(Oc2ccc(N3CCC[C@@H]3c3csc(NC(=O)c4cccn4Cc4ccnc(F)c4)n3)cc2)CC1] |
| A129 | 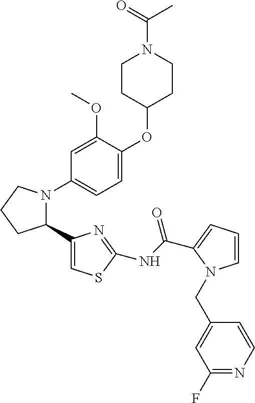 [SMILES: COc1cc(N2CCC[C@@H]2c2csc(NC(=O)c3cccn3Cc3ccnc(F)c3)n2)ccc1OC1CCN(C(C)=O)CC1] |
| A130 | 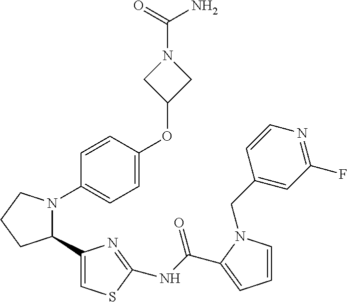 [SMILES: NC(=O)N1CC(Oc2ccc(N3CCC[C@@H]3c3csc(NC(=O)c4cccn4Cc4ccnc(F)c4)n3)cc2)C1] |
| A131 | 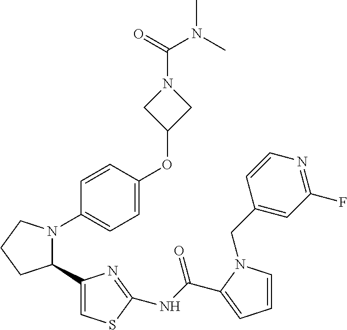 [SMILES: CN(C)C(=O)N1CC(Oc2ccc(N3CCC[C@@H]3c3csc(NC(=O)c4cccn4Cc4ccnc(F)c4)n3)cc2)C1] |
| A132 | 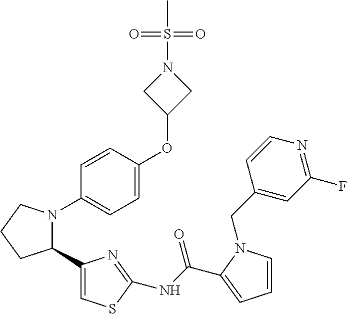 [SMILES: CS(=O)(=O)N1CC(Oc2ccc(N3CCC[C@@H]3c3csc(NC(=O)c4cccn4Cc4ccnc(F)c4)n3)cc2)C1] |
| A133 | 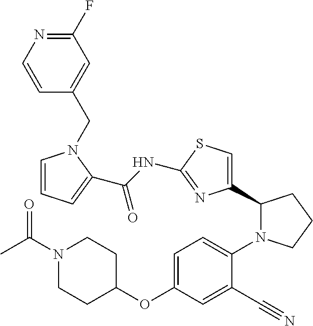 [SMILES: CC(=O)N1CCC(Oc2ccc(N3CCC[C@@H]3c3csc(NC(=O)c4cccn4Cc4ccnc(F)c4)n3)c(C#N)c2)CC1] |
| A134 | 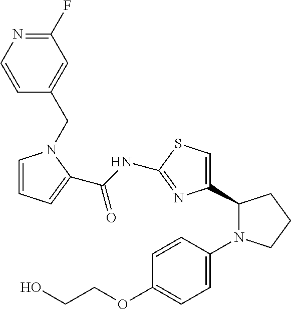 [SMILES: O=C(Nc1nc([C@H]2CCCN2c2ccc(OCCO)cc2)cs1)c1cccn1Cc1ccnc(F)c1] |
| A135 | 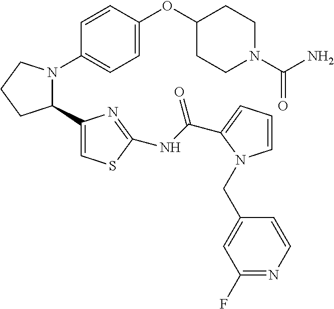 [SMILES: NC(=O)N1CCC(Oc2ccc(N3CCC[C@@H]3c3csc(NC(=O)c4cccn4Cc4ccnc(F)c4)n3)cc2)CC1] |
| A136 | 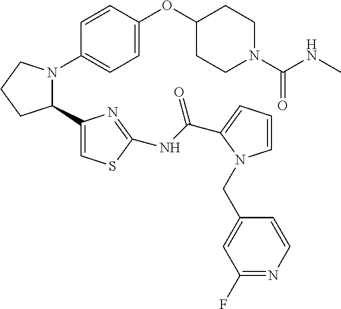 [SMILES: CNC(=O)N1CCC(Oc2ccc(N3CCC[C@@H]3c3csc(NC(=O)c4cccn4Cc4ccnc(F)c4)n3)cc2)CC1] |
| A137 | 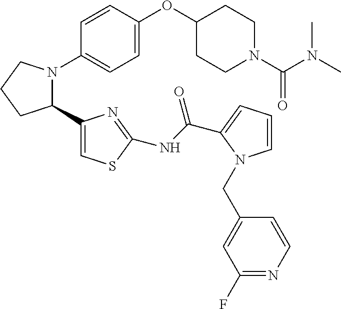 [SMILES: CN(C)C(=O)N1CCC(Oc2ccc(N3CCC[C@@H]3c3csc(NC(=O)c4cccn4Cc4ccnc(F)c4)n3)cc2)CC1] |
| A138 | 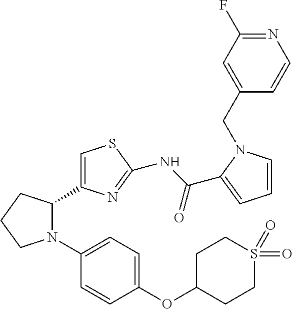 [SMILES: O=C(Nc1nc([C@H]2CCCN2c2ccc(OC3CCS(=O)(=O)CC3)cc2)cs1)c1cccn1Cc1ccnc(F)c1] |
| A139 | 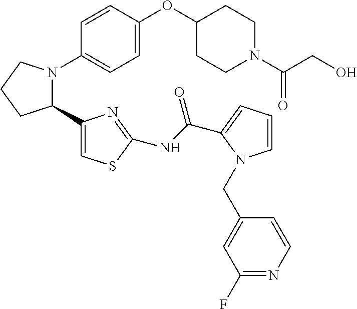 [SMILES: O=C(Nc1nc([C@H]2CCCN2c2ccc(OC3CCN(C(=O)CO)CC3)cc2)cs1)c1cccn1Cc1ccnc(F)c1] |
| A140 | 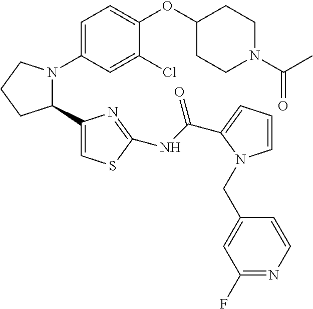 [SMILES: CC(=O)N1CCC(Oc2ccc(N3CCC[C@@H]3c3csc(NC(=O)c4cccn4Cc4ccnc(F)c4)n3)cc2Cl)CC1] |
| A141 | 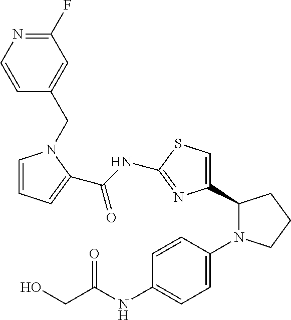 [SMILES: O=C(CO)Nc1ccc(N2CCC[C@@H]2c2csc(NC(=O)c3cccn3Cc3ccnc(F)c3)n2)cc1] |
| A142 | 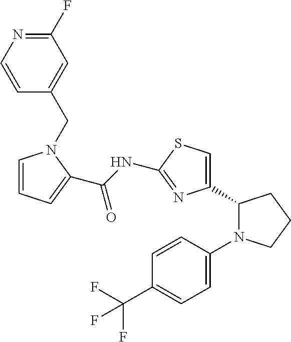 [SMILES: O=C(Nc1nc([C@@H]2CCCN2c2ccc(C(F)(F)F)cc2)cs1)c1cccn1Cc1ccnc(F)c1] |
| A143 | 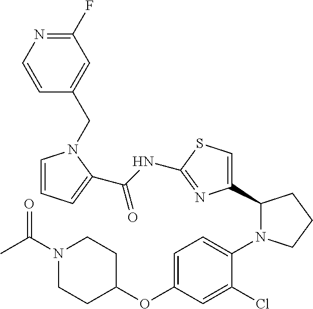 [SMILES: CC(=O)N1CCC(Oc2ccc(N3CCC[C@@H]3c3csc(NC(=O)c4cccn4Cc4ccnc(F)c4)n3)c(Cl)c2)CC1] |
| A144 | 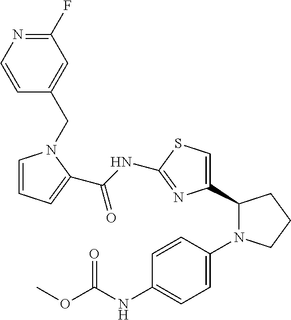 [SMILES: COC(=O)Nc1ccc(N2CCC[C@@H]2c2csc(NC(=O)c3cccn3Cc3ccnc(F)c3)n2)cc1] |
| A145 | 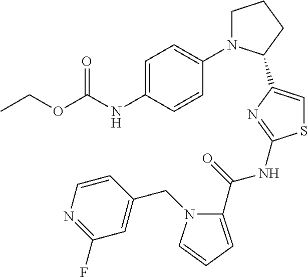 [SMILES: CCOC(=O)Nc1ccc(N2CCC[C@@H]2c2csc(NC(=O)c3cccn3Cc3ccnc(F)c3)n2)cc1] |
| A146 | 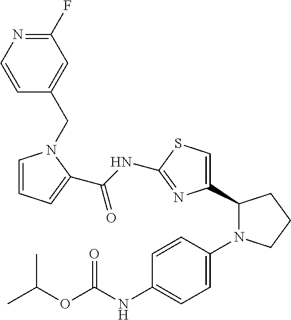 [SMILES: CC(C)OC(=O)Nc1ccc(N2CCC[C@@H]2c2csc(NC(=O)c3cccn3Cc3ccnc(F)c3)n2)cc1] |
| A147 | 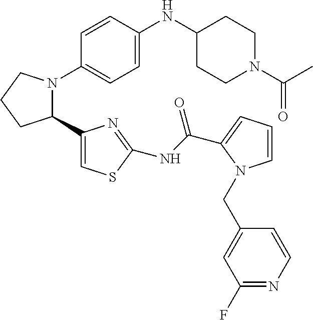 [SMILES: CC(=O)N1CCC(Nc2ccc(N3CCC[C@@H]3c3csc(NC(=O)c4cccn4Cc4ccnc(F)c4)n3)cc2)CC1] |
| A148 | 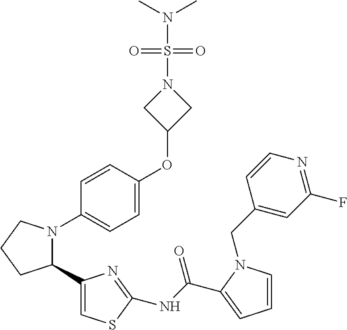 [SMILES: CN(C)S(=O)(=O)N1CC(Oc2ccc(N3CCC[C@@H]3c3csc(NC(=O)c4cccn4Cc4ccnc(F)c4)n3)cc2)C1] |
| A149 | 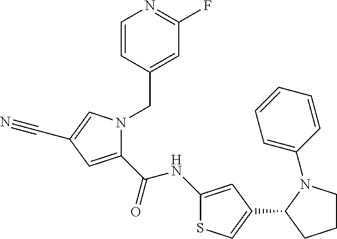 [SMILES: N#Cc1cc(C(=O)Nc2cc([C@H]3CCCN3c3ccccc3)cs2)n(Cc2ccnc(F)c2)c1] |
| A150 | 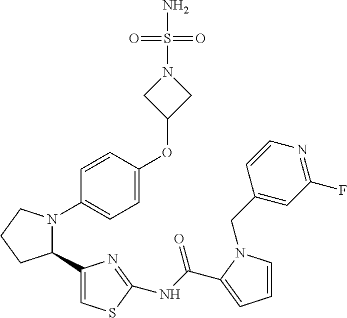 [SMILES: NS(=O)(=O)N1CC(Oc2ccc(N3CCC[C@@H]3c3csc(NC(=O)c4cccn4Cc4ccnc(F)c4)n3)cc2)C1] |
| A151 | 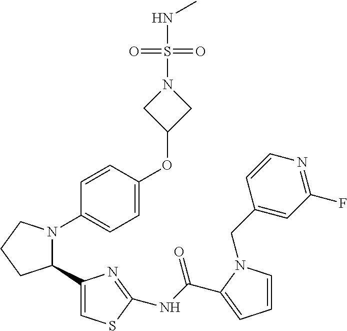 [SMILES: CNS(=O)(=O)N1CC(Oc2ccc(N3CCC[C@@H]3c3csc(NC(=O)c4cccn4Cc4ccnc(F)c4)n3)cc2)C1] |
| A152 | 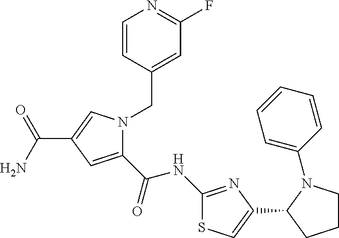 [SMILES: NC(=O)c1cc(C(=O)Nc2nc([C@H]3CCCN3c3ccccc3)cs2)n(Cc2ccnc(F)c2)c1] |
| A153 | 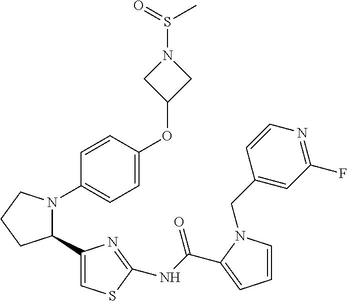 [SMILES: CS(=O)N1CC(Oc2ccc(N3CCC[C@@H]3c3csc(NC(=O)c4cccn4Cc4ccnc(F)c4)n3)cc2)C1] |
| A154 | 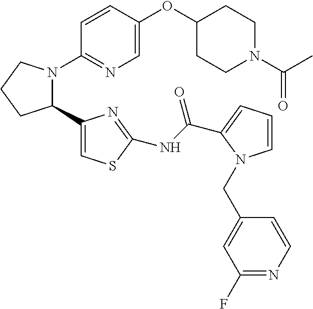 [SMILES: CC(=O)N1CCC(Oc2ccc(N3CCC[C@@H]3c3csc(NC(=O)c4cccn4Cc4ccnc(F)c4)n3)nc2)CC1] |
| A155 | 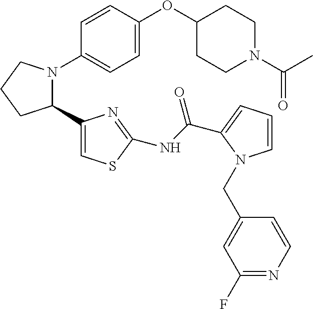 [SMILES: CC(=O)N1CCC(Oc2ccc(N3CCC[C@@H]3c3csc(NC(=O)c4cccn4Cc4ccnc(F)c4)n3)cc2)CC1] |
| A156 | 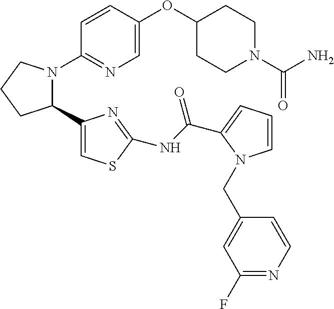 [SMILES: NC(=O)N1CCC(Oc2ccc(N3CCC[C@@H]3c3csc(NC(=O)c4cccn4Cc4ccnc(F)c4)n3)nc2)CC1] |
| A157 | 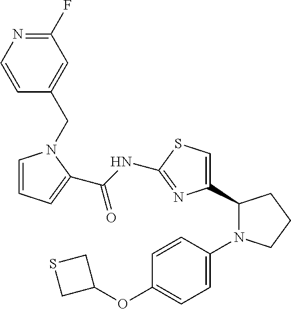 [SMILES: O=C(Nc1nc([C@H]2CCCN2c2ccc(OC3CSC3)cc2)cs1)c1cccn1Cc1ccnc(F)c1] |
| A158 | 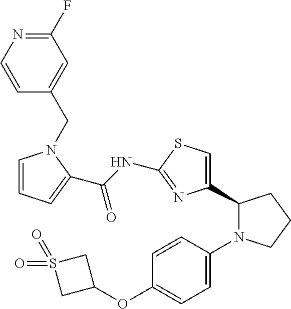 [SMILES: O=C(Nc1nc([C@H]2CCCN2c2ccc(OC3CS(=O)(=O)C3)cc2)cs1)c1cccn1Cc1ccnc(F)c1] |
| A159 | 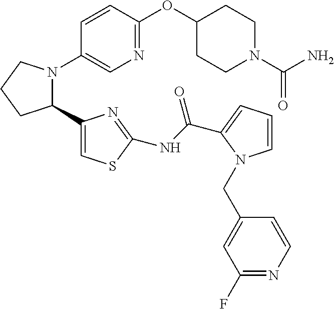 [SMILES: NC(=O)N1CCC(Oc2ccc(N3CCC[C@@H]3c3csc(NC(=O)c4cccn4Cc4ccnc(F)c4)n3)cn2)CC1] |
| A160 | 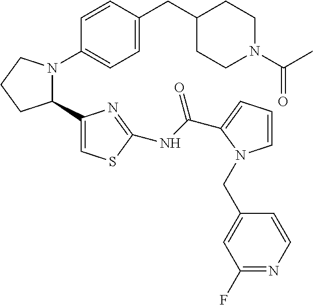 [SMILES: CC(=O)N1CCC(Cc2ccc(N3CCC[C@@H]3c3csc(NC(=O)c4cccn4Cc4ccnc(F)c4)n3)cc2)CC1] |
| A161 | 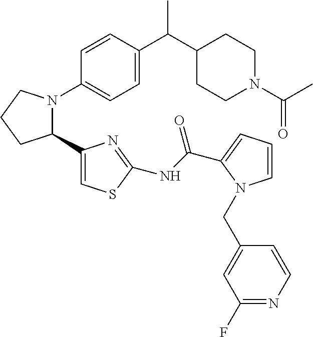 [SMILES: CC(=O)N1CCC(C(C)c2ccc(N3CCC[C@@H]3c3csc(NC(=O)c4cccn4Cc4ccnc(F)c4)n3)cc2)CC1] |
| A162 | 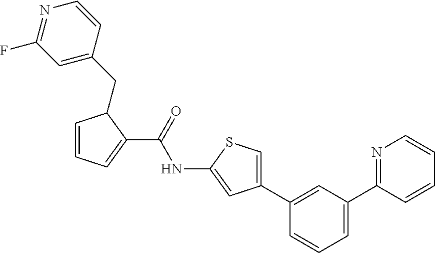 [SMILES: O=C(Nc1cc(-c2cccc(-c3ccccn3)c2)cs1)C1=CC=CC1Cc1ccnc(F)c1] |
| A163 | 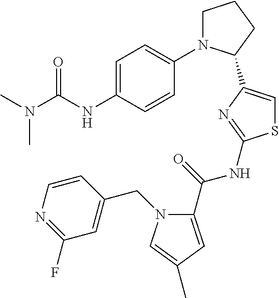 [SMILES: Cc1cc(C(=O)Nc2nc([C@H]3CCCN3c3ccc(NC(=O)N(C)C)cc3)cs2)n(Cc2ccnc(F)c2)c1] |
| A164 | 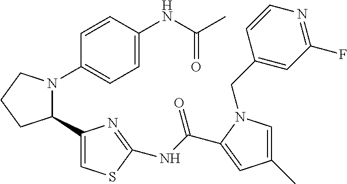 [SMILES: CC(=O)Nc1ccc(N2CCC[C@@H]2c2csc(NC(=O)c3cc(C)cn3Cc3ccnc(F)c3)n2)cc1] |
| A165 | 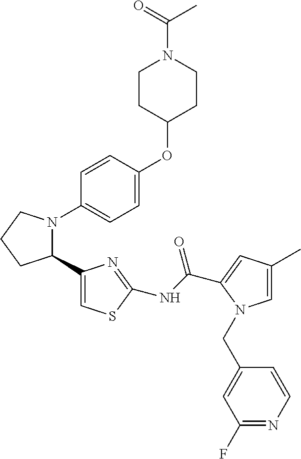 [SMILES: CC(=O)N1CCC(Oc2ccc(N3CCC[C@@H]3c3csc(NC(=O)c4cc(C)cn4Cc4ccnc(F)c4)n3)cc2)CC1] |
| A166 | 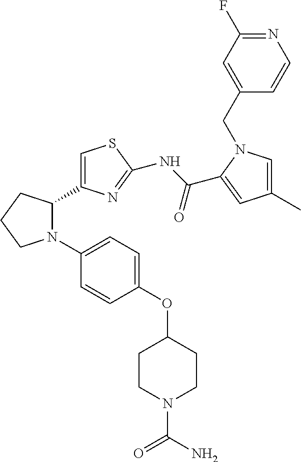 [SMILES: Cc1cc(C(=O)Nc2nc([C@H]3CCCN3c3ccc(OC4CCN(C(N)=O)CC4)cc3)cs2)n(Cc2ccnc(F)c2)c1] |
| A167 | 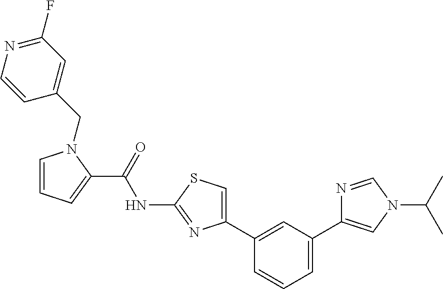 [SMILES: CC(C)n1cnc(-c2cccc(-c3csc(NC(=O)c4cccn4Cc4ccnc(F)c4)n3)c2)c1] |
| A168 | 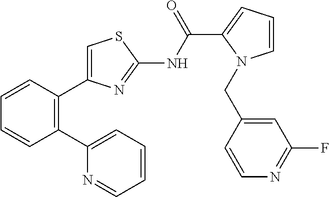 [SMILES: O=C(Nc1nc(-c2ccccc2-c2ccccn2)cs1)c1cccn1Cc1ccnc(F)c1] |
| A169 | 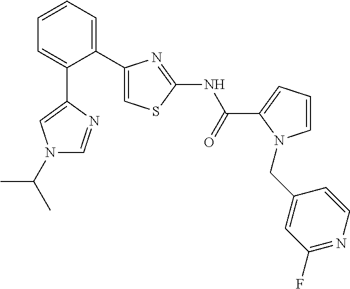 [SMILES: CC(C)n1cnc(-c2ccccc2-c2csc(NC(=O)c3cccn3Cc3ccnc(F)c3)n2)c1] |
| A170 | 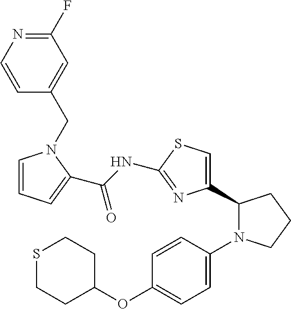 [SMILES: O=C(Nc1nc([C@H]2CCCN2c2ccc(OC3CCSCC3)cc2)cs1)c1cccn1Cc1ccnc(F)c1] |
| A171 | 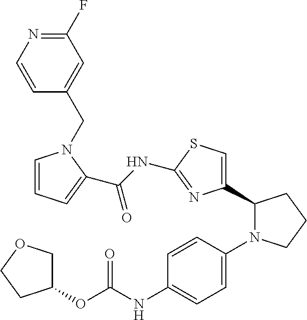 [SMILES: O=C(Nc1ccc(N2CCC[C@@H]2c2csc(NC(=O)c3cccn3Cc3ccnc(F)c3)n2)cc1)O[C@@H]1CCOC1] |
| A172 | 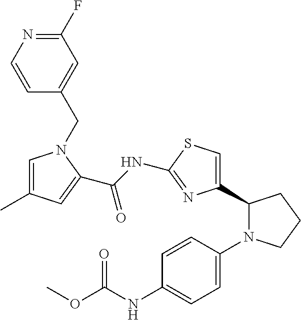 [SMILES: COC(=O)Nc1ccc(N2CCC[C@@H]2c2csc(NC(=O)c3cc(C)cn3Cc3ccnc(F)c3)n2)cc1] |
| A173 | 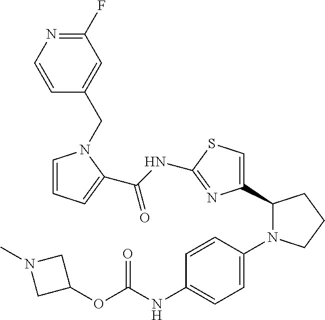 [SMILES: CN1CC(OC(=O)Nc2ccc(N3CCC[C@@H]3c3csc(NC(=O)c4cccn4Cc4ccnc(F)c4)n3)cc2)C1] |
| A174 | 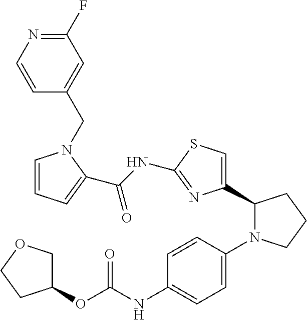 [SMILES: O=C(Nc1ccc(N2CCC[C@@H]2c2csc(NC(=O)c3cccn3Cc3ccnc(F)c3)n2)cc1)O[C@H]1CCOC1] |
| A175 | 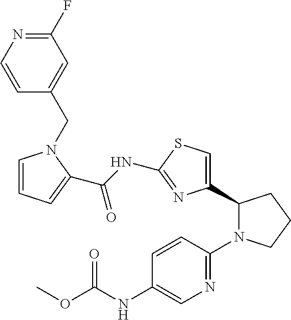 [SMILES: COC(=O)Nc1ccc(N2CCC[C@@H]2c2csc(NC(=O)c3cccn3Cc3ccnc(F)c3)n2)nc1] |
| A176 | 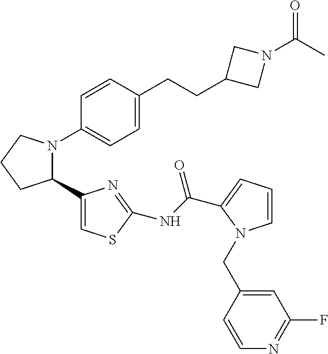 [SMILES: CC(=O)N1CC(CCc2ccc(N3CCC[C@@H]3c3csc(NC(=O)c4cccn4Cc4ccnc(F)c4)n3)cc2)C1] |
| A177 | 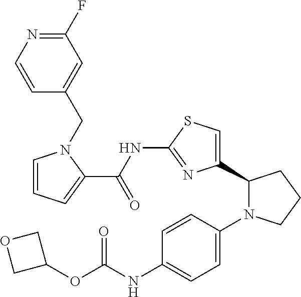 [SMILES: O=C(Nc1ccc(N2CCC[C@@H]2c2csc(NC(=O)c3cccn3Cc3ccnc(F)c3)n2)cc1)OC1COC1] |
| A178 | 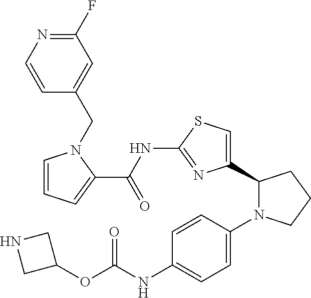 [SMILES: O=C(Nc1ccc(N2CCC[C@@H]2c2csc(NC(=O)c3cccn3Cc3ccnc(F)c3)n2)cc1)OC1CNC1] |
| A179 | 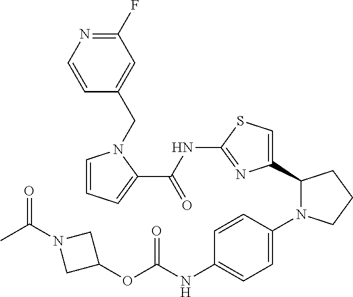 [SMILES: CC(=O)N1CC(OC(=O)Nc2ccc(N3CCC[C@@H]3c3csc(NC(=O)c4cccn4Cc4ccnc(F)c4)n3)cc2)C1] |
| A180 | 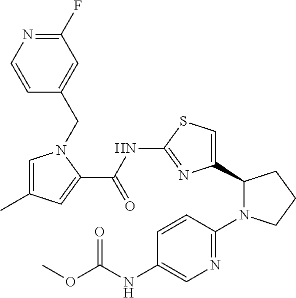 [SMILES: COC(=O)Nc1ccc(N2CCC[C@@H]2c2csc(NC(=O)c3cc(C)cn3Cc3ccnc(F)c3)n2)nc1] |
| A181 | 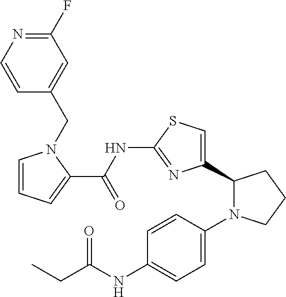 [SMILES: CCC(=O)Nc1ccc(N2CCC[C@@H]2c2csc(NC(=O)c3cccn3Cc3ccnc(F)c3)n2)cc1] |
| A182 | 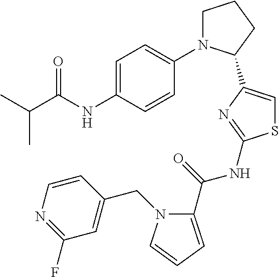 [SMILES: CC(C)C(=O)Nc1ccc(N2CCC[C@@H]2c2csc(NC(=O)c3cccn3Cc3ccnc(F)c3)n2)cc1] |
| A183 | 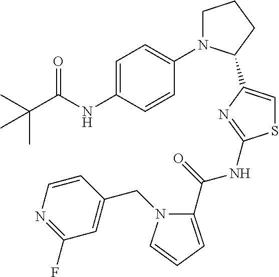 [SMILES: CC(C)(C)C(=O)Nc1ccc(N2CCC[C@@H]2c2csc(NC(=O)c3cccn3Cc3ccnc(F)c3)n2)cc1] |
| A184 | 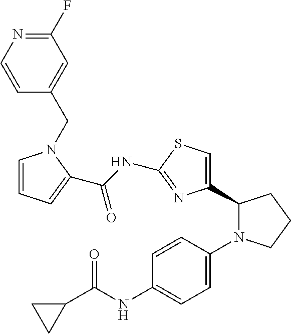 [SMILES: O=C(Nc1nc([C@H]2CCCN2c2ccc(NC(=O)C3CC3)cc2)cs1)c1cccn1Cc1ccnc(F)c1] |
| A185 | 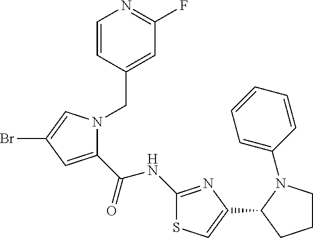 [SMILES: O=C(Nc1nc([C@H]2CCCN2c2ccccc2)cs1)c1cc(Br)cn1Cc1ccnc(F)c1] |
| A186 | 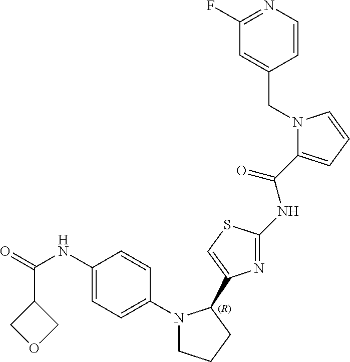 |
| A187 | 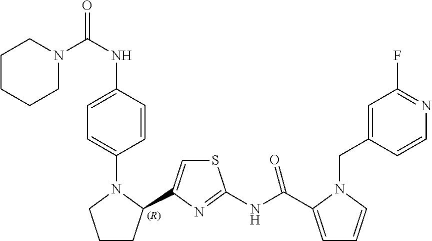 |
| A188 | 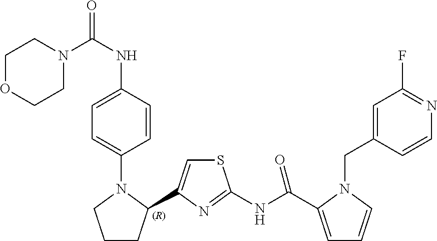 |
| A189 | 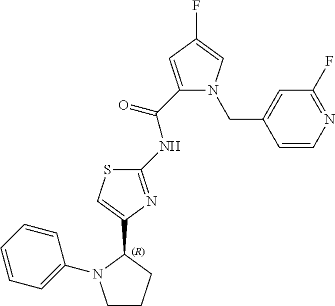 |
| A190 | 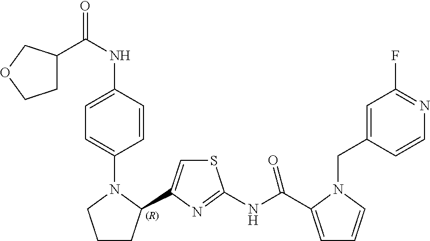 |
| A191 | 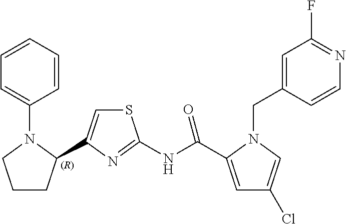 |
| A192 | 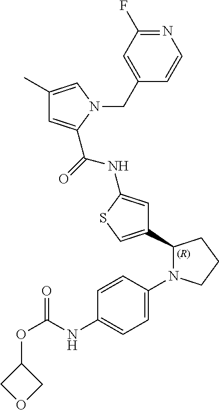 |
| A193 | 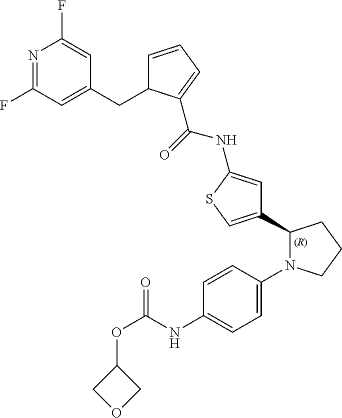 |
| A194 | 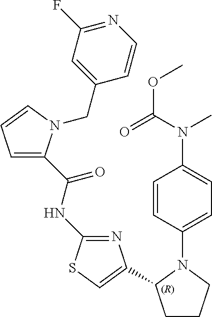 |
| A195 | 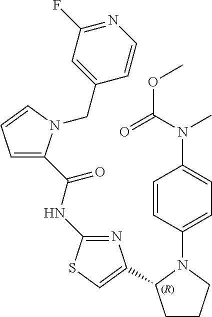 |
| A196 | 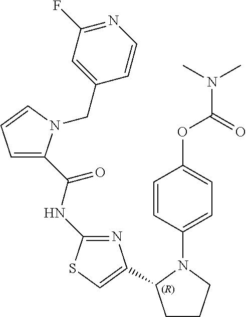 |
| A197 | 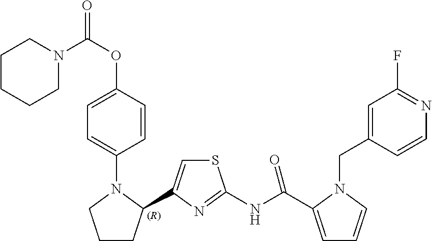 |
| A198 | 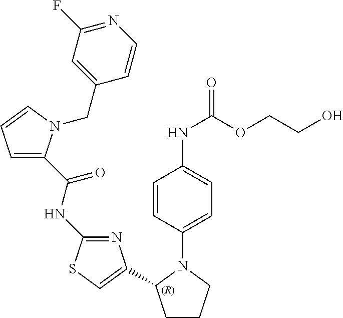 |
| A199 | 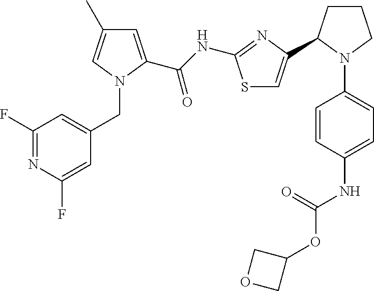 [SMILES: Cc1cc(C(=O)Nc2nc([C@H]3CCCN3c3ccc(NC(=O)OC4COC4)cc3)cs2)n(Cc2cc(F)nc(F)c2)c1] |
| A200 | 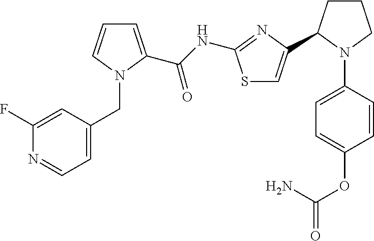 [SMILES: NC(=O)Oc1ccc(N2CCC[C@@H]2c2csc(NC(=O)c3cccn3Cc3ccnc(F)c3)n2)cc1] |
| A201 | 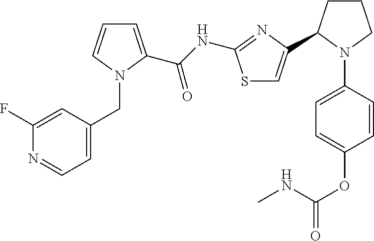 [SMILES: CNC(=O)Oc1ccc(N2CCC[C@@H]2c2csc(NC(=O)c3cccn3Cc3ccnc(F)c3)n2)cc1] |
| A202 | 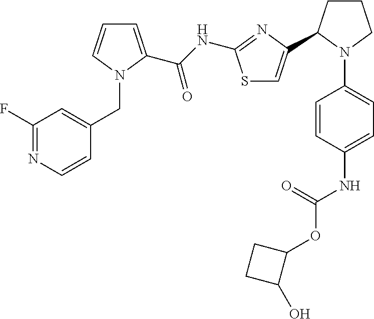 [SMILES: O=C(Nc1ccc(N2CCC[C@@H]2c2csc(NC(=O)c3cccn3Cc3ccnc(F)c3)n2)cc1)OC1CCC1O] |
| A203 | 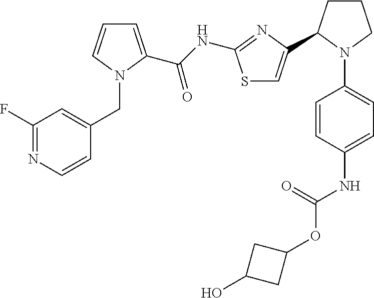 [SMILES: O=C(Nc1ccc(N2CCC[C@@H]2c2csc(NC(=O)c3cccn3Cc3ccnc(F)c3)n2)cc1)OC1CC(O)C1] |
| A204 | 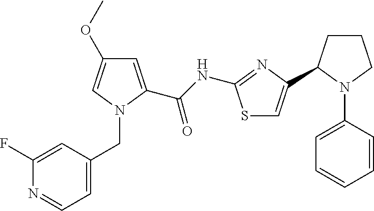 [SMILES: COc1cc(C(=O)Nc2nc([C@H]3CCCN3c3ccccc3)cs2)n(Cc2ccnc(F)c2)c1] |
| A205 | 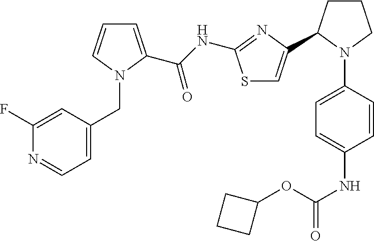 [SMILES: O=C(Nc1ccc(N2CCC[C@@H]2c2csc(NC(=O)c3cccn3Cc3ccnc(F)c3)n2)cc1)OC1CCC1] |
| A206 | 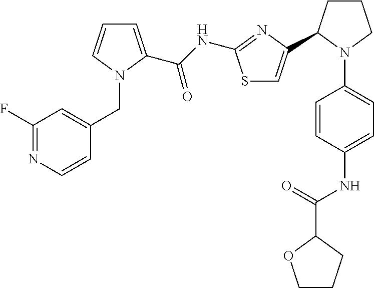 [SMILES: O=C(Nc1nc([C@H]2CCCN2c2ccc(NC(=O)C3CCCO3)cc2)cs1)c1cccn1Cc1ccnc(F)c1] |
| A207 | 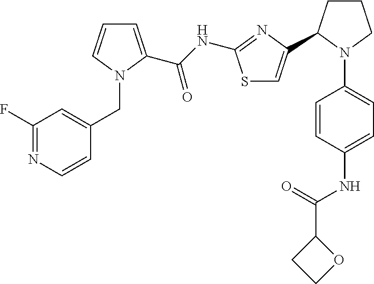 [SMILES: O=C(Nc1nc([C@H]2CCCN2c2ccc(NC(=O)C3CCO3)cc2)cs1)c1cccn1Cc1ccnc(F)c1] |
| A208 | 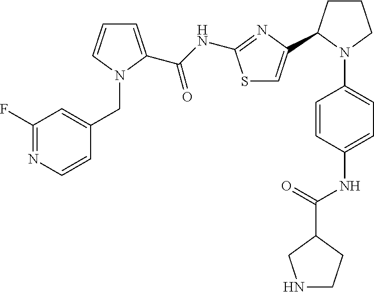 [SMILES: O=C(Nc1nc([C@H]2CCCN2c2ccc(NC(=O)C3CCNC3)cc2)cs1)c1cccn1Cc1ccnc(F)c1] |
| A209 | 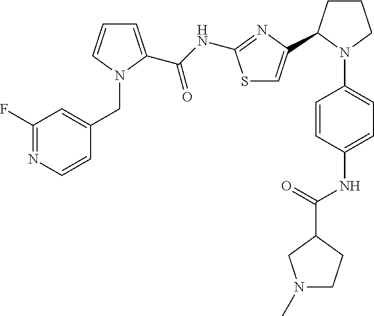 [SMILES: CN1CCC(C(=O)Nc2ccc(N3CCC[C@@H]3c3csc(NC(=O)c4cccn4Cc4ccnc(F)c4)n3)cc2)C1] |
| A210 | 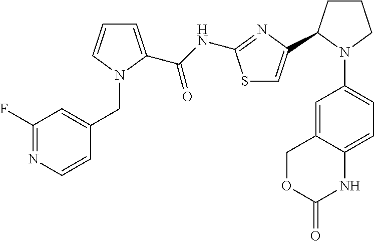 [SMILES: O=C1Nc2ccc(N3CCC[C@@H]3c3csc(NC(=O)c4cccn4Cc4ccnc(F)c4)n3)cc2CO1] |
| E19 | 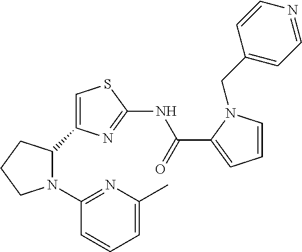 |
| E22 | 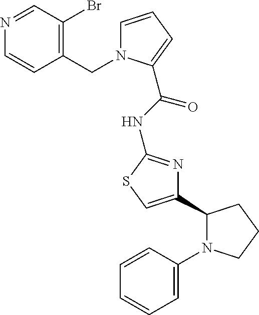 [SMILES: O=C(Nc1nc([C@H]2CCCN2c2ccccc2)cs1)c1cccn1Cc1ccncc1Br] |
| E23 | 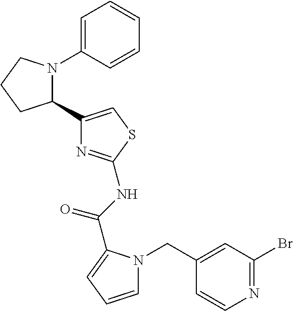 [SMILES: O=C(Nc1nc([C@H]2CCCN2c2ccccc2)cs1)c1cccn1Cc1ccnc(Br)c1] |
| E24 | 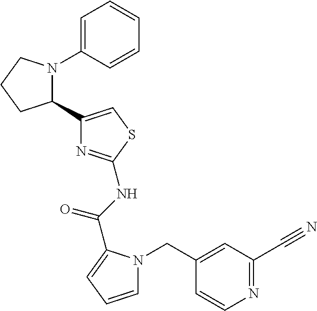 [SMILES: N#Cc1cc(Cn2cccc2C(=O)Nc2nc([C@H]3CCCN3c3ccccc3)cs2)ccn1] |
| E25 | 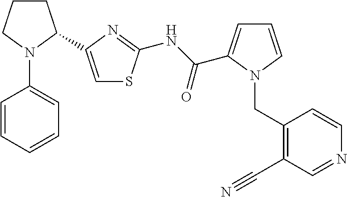 [SMILES: N#Cc1cnccc1Cn1cccc1C(=O)Nc1nc([C@H]2CCCN2c2ccccc2)cs1] |
| E26 | 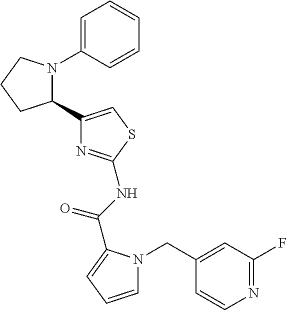 [SMILES: O=C(Nc1nc([C@H]2CCCN2c2ccccc2)cs1)c1cccn1Cc1ccnc(F)c1] |
| E27 | 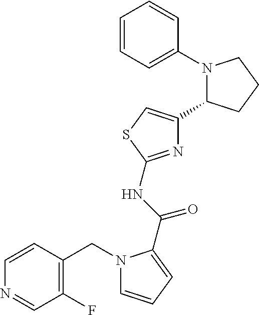 [SMILES: O=C(Nc1nc([C@H]2CCCN2c2ccccc2)cs1)c1cccn1Cc1ccncc1F] |
| E28 | 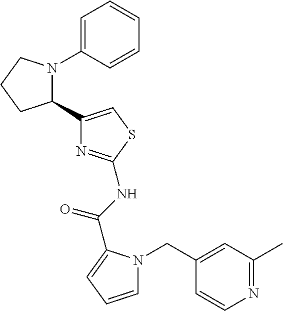 [SMILES: Cc1cc(Cn2cccc2C(=O)Nc2nc([C@H]3CCCN3c3ccccc3)cs2)ccn1] |
| E29 | 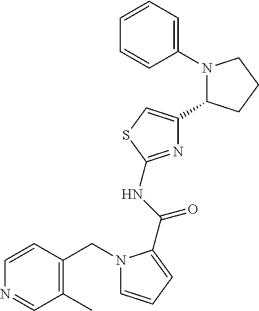 [SMILES: Cc1cnccc1Cn1cccc1C(=O)Nc1nc([C@H]2CCCN2c2ccccc2)cs1] |
| E59 | 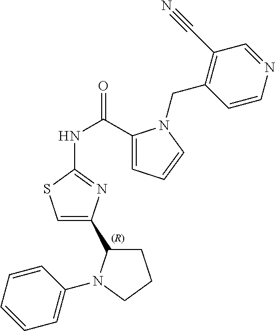 |
| E60 | 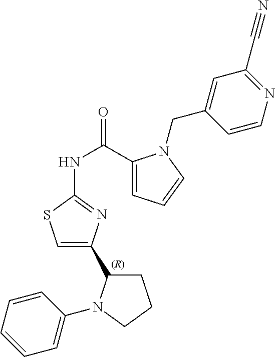 |
| E62 | 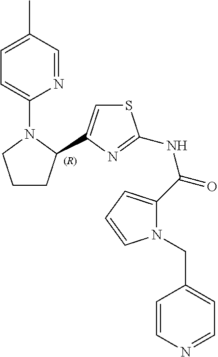 |
| E63 | 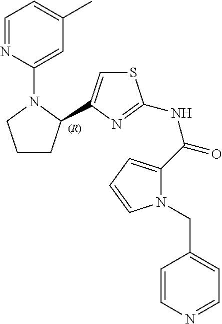 |
| E64 | 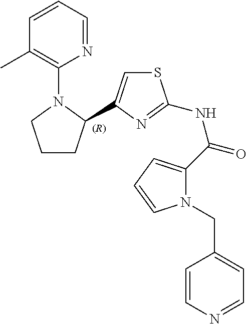 |
| E65 | 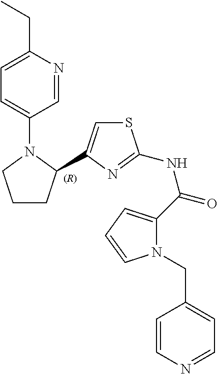 |

### Compounds of Formula (II)

Also provided herein are compounds or pharmaceutically acceptable salt thereof, having a structure of formula (II):

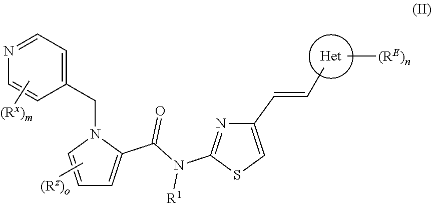

wherein
R¹ is H, C₁₋₃alkyl, or SO₂C₁₋₆alkyl;
Het is oxazole, imidazole, pyrazole, isoxazole, morpholine, tetrahydroquinoline, oxazolidinone, piperidinone, dihydrooxazole, pyrazine, pyrimidine, imidazo[1,2-a]pyridine, 5,6,7,8-tetrahydroimidazo[1,5-a]pyridine, pyridine-2(1H)-one, 6,7-dihydro-5H-pyrrolo[1,2-a]imidazole, or quinoline, or
when at least one of n and m is 1 or 2, Het can be pyridine, and when n is 1 or 2, Het can be diazinyl;
n is 0, 1, or 2;
each R^{E}, when present, is independently halo, C₁₋₆alkyl, C₀₋₆alkylene-C(O)N(R^{N})₂, C₀₋₆alkylene-N(R^{N})C(O)R^{N}, C₀₋₆alkylene-CN, C₀₋₆alkylene-OR^{N}, C₀₋₆alkylene-N(R^{N})₂, C₁₋₆haloalkyl, C₁₋₆haloalkoxy, C₁₋₆ hydroxyalkyl, C₀₋₆alkylene-CO₂R^{N}, or C₀₋₆alkylene-[C(O)]₀₋₁-3-6 membered aromatic or non-aromatic ring having 0-2 ring heteroatoms independently selected from N, O and S;
   wherein when R^{E} comprises a 3-6 membered ring, it is optionally substituted with 1-2 groups independently selected from halo, C₁₋₆alkyl, CN, C₁₋₆haloalkyl, CO₂R^{N}, C(O)R^{N}, CON(R^{N})₂, N(R^{N})COR^{N}, and OR^{N};
m is 0, 1, or 2;
each R^{x}, when present, is independently halo or C₁₋₆alkyl;
o is 0 or 1;
R^{z}, when present, is CN, halo, C(O)N(R^{N})₂, C₁₋₆alkyl, C₁₋₆alkoxy, C₁₋₆hydroxyalkyl, or C₁₋₆haloalkyl; and
each R^{N} is independently H, C₁₋₆alkyl, C₁₋₆hydroxyalkyl, or C₁₋₆haloalkyl.

In various cases, R¹ is H. In some cases, Het is imidazole or oxazole. In various cases, Het is oxazole. In various cases, Het is imidazole. In various cases, when n is 1 or 2, Het is diazinyl. In various cases, Het is isoxazole, morpholine, tetrahydroquinoline, oxazolindinone, piperidinone, or dihydrooxazole. In various cases, Het is pyrazine, pyrimidine, imidazo[1,2-a]pyridine, 5,6,7,8-tetrahydroimidazo[1,5-a]pyridine, pyridine-2(1H)-one, 6,7-dihydro-5H-pyrrolo[1,2-a]imidazole, or quinolone. In some cases, where at least one of n and m is 1 or 2, Het is pyridine.

In various cases, n is 0. In various cases, n is 1 or 2. In some cases, n is 1. In some cases, n is 2. In cases where n is 1 or 2, in some cases at least one R^{E} is halo (e.g., fluoro). In cases where n is 1 or 2, in some cases at least one R^{E} is C₁₋₆alkyl or C(O)N(R^{N})₂. In cases where n is 1 or 2, in some cases at least one R^{E} is C₁₋₆alkyl or C₀₋₆alkylene-CN. In cases where n is 1 or 2, in some cases at least one R^{E} is phenyl—and in some cases, the phenyl is unsubstituted. In some case, the phenyl is substituted with 1 substituent selected from halo, C₁₋₆haloalkyl, C₁₋₆haloalkoxy, CON(R^{N})₂, N(R^{N})COR^{N} and OR^{N}. In some cases, at least one R^{E} is C₁₋₆alkylene-C(O)N(R^{N})₂, C₁₋₆alkylene-CN, C₁₋₆hydroxyalkyl, 3-6 membered heterocycloalkyl having 1 or 2 heteroatoms independently selected from N, O and S, or C₁₋₆alkylene-CO₂R^{N}. In some cases, the 3-6 membered heterocycloalkyl is unsubstituted. In some cases, the 3-6 membered heterocycloalkyl is substituted, and in some specific cases, the substituent is halo, C₁₋₆alkyl, CN, C₁₋₆haloalkyl, C₁₋₆haloalkoxy, CO₂R^{N}, C(O)R^{N}, CON(R^{N})₂, N(R^{N})COR^{N}, or OR^{N}.

In various cases, m is 0. In some cases, m is 1 or 2. In some cases when m is 1, R^{x} is at 2-position of pyridine, i.e.,

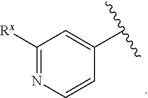

In some cases, m is 2, and in some specific cases, one R^{x} is at 2-position and other R^{x} is at 6-position of pyridine, i.e.,

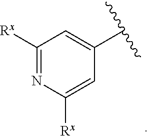

In various cases, R^{x} is halo or methyl. In some cases, at least one R^{x} is fluoro. In some cases, when m is 2, each R^{x} is fluoro.

In various cases, o is 0. In some cases, o is 1, and in some specific cases, R^{z} is meta to the ring nitrogen, i.e.,

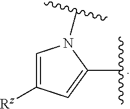

In some cases, R^{z} is methyl or fluoro.

In various cases, each R^{N} is independently H or methyl. In some cases, at least one R^{N} is C₁₋₆hydroxyalkyl or C₁₋₆haloalkyl.

In various cases, the compound of Formula (II) is a structure as shown in Table B, or a pharmaceutically acceptable salt thereof:

TABLE B

| ID | |
|---|---|
| B1 | 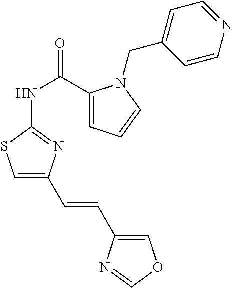 [SMILES: O=C(Nc1nc(/C=C/c2cocn2)cs1)c1cccn1Cc1ccncc1] |
| B2 | 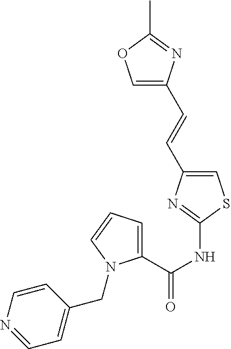 [SMILES: Cc1nc(/C=C/c2csc(NC(=O)c3cccn3Cc3ccncc3)n2)co1] |
| B3 | 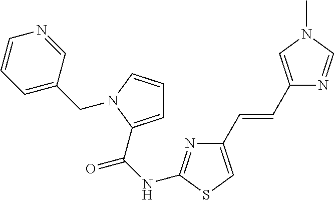 [SMILES: Cn1cnc(/C=C/c2csc(NC(=O)c3cccn3Cc3cccnc3)n2)c1] |
| B4 | 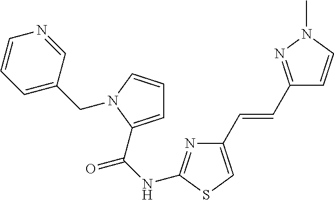 [SMILES: Cn1ccc(/C=C/c2csc(NC(=O)c3cccn3Cc3cccnc3)n2)n1] |
| B5 | 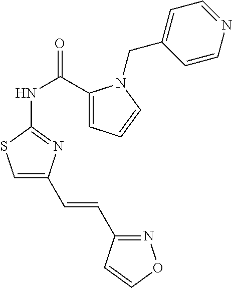 [SMILES: O=C(Nc1nc(/C=C/c2ccon2)cs1)c1cccn1Cc1ccncc1] |
| B6 | 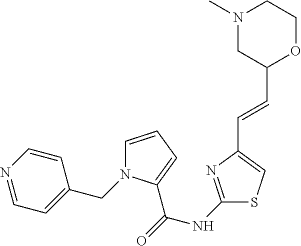 [SMILES: CN1CCOC(/C=C/c2csc(NC(=O)c3cccn3Cc3ccncc3)n2)C1] |
| B7 | 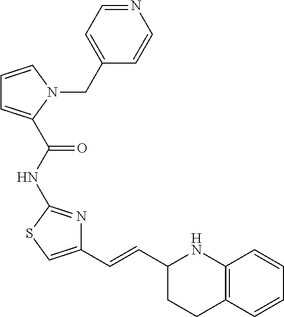 [SMILES: O=C(Nc1nc(/C=C/C2CCc3ccccc3N2)cs1)c1cccn1Cc1ccncc1] |
| B8 | 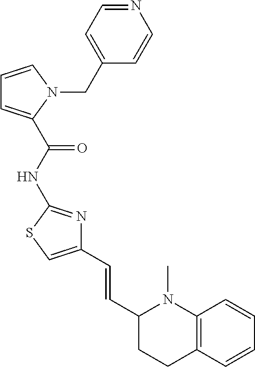 [SMILES: CN1c2ccccc2CCC1/C=C/c1csc(NC(=O)c2cccn2Cc2ccncc2)n1] |
| B9 | 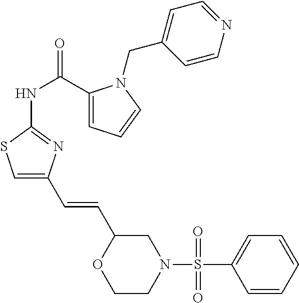 [SMILES: O=C(Nc1nc(/C=C/C2CN(S(=O)(=O)c3ccccc3)CCO2)cs1)c1cccn1Cc1ccncc1] |
| B10 | 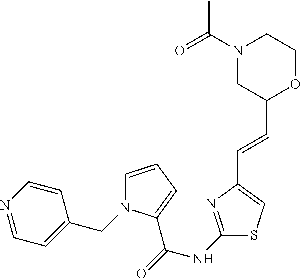 [SMILES: CC(=O)N1CCOC(/C=C/c2csc(NC(=O)c3cccn3Cc3ccncc3)n2)C1] |
| B11 | 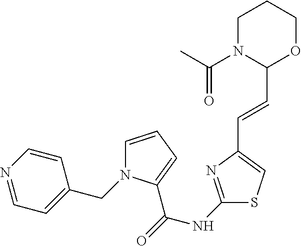 [SMILES: CC(=O)N1CCCOC1/C=C/c1csc(NC(=O)c2cccn2Cc2ccncc2)n1] |
| B12 | 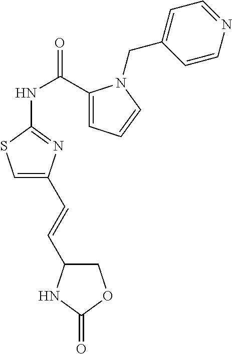 [SMILES: O=C1NC(/C=C/c2csc(NC(=O)c3cccn3Cc3ccncc3)n2)CO1] |
| B13 | 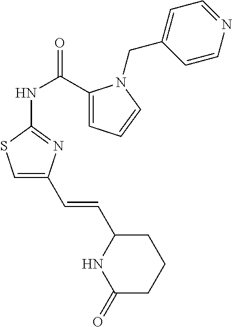 [SMILES: O=C1CCCC(/C=C/c2csc(NC(=O)c3cccn3Cc3ccncc3)n2)N1] |
| B14 | 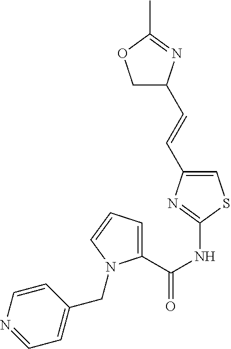 [SMILES: CC1=NC(/C=C/c2csc(NC(=O)c3cccn3Cc3ccncc3)n2)CO1] |
| B15 | 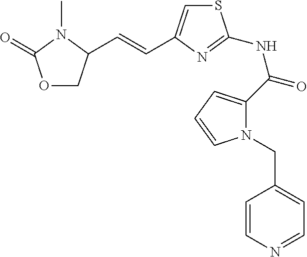 [SMILES: CN1C(=O)OCC1/C=C/c1csc(NC(=O)c2cccn2Cc2ccncc2)n1] |
| B16 | 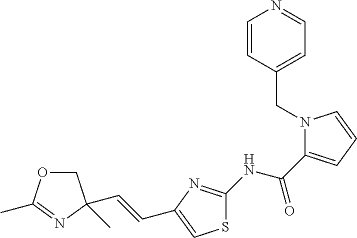 [SMILES: CC1=NC(C)(/C=C/c2csc(NC(=O)c3cccn3Cc3ccncc3)n2)CO1] |
| B17 | 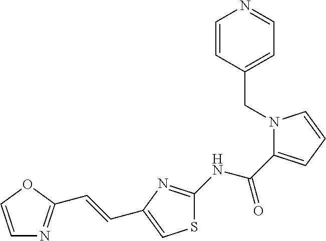 [SMILES: O=C(Nc1nc(/C=C/c2ncco2)cs1)c1cccn1Cc1ccncc1] |
| B18 | 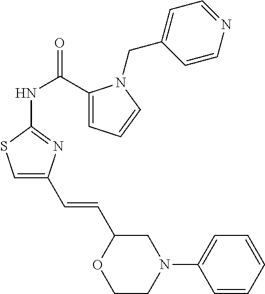 [SMILES: O=C(Nc1nc(/C=C/C2CN(c3ccccc3)CCO2)cs1)c1cccn1Cc1ccncc1] |
| B19 | 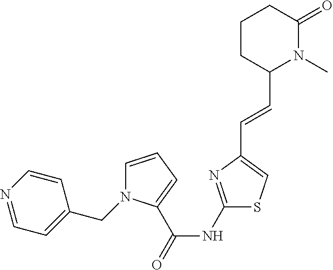 [SMILES: CN1C(=O)CCCC1/C=C/c1csc(NC(=O)c2cccn2Cc2ccncc2)n1] |
| B20 | 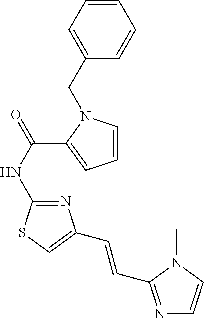 [SMILES: Cn1ccnc1/C=C/c1csc(NC(=O)c2cccn2Cc2ccccc2)n1] |
| B21 | 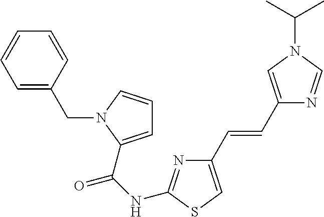 [SMILES: CC(C)n1cnc(/C=C/c2csc(NC(=O)c3cccn3Cc3ccccc3)n2)c1] |
| B22 | 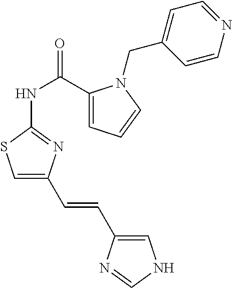 [SMILES: O=C(Nc1nc(/C=C/c2c[nH]cn2)cs1)c1cccn1Cc1ccncc1] |
| B23 | 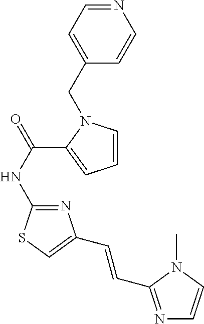 [SMILES: Cn1ccnc1/C=C/c1csc(NC(=O)c2cccn2Cc2ccncc2)n1] |
| B24 | 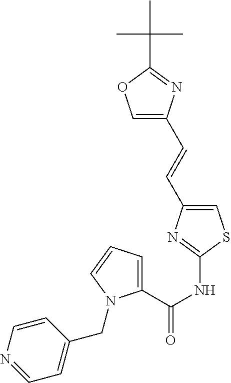 [SMILES: CC(C)(C)c1nc(/C=C/c2csc(NC(=O)c3cccn3Cc3ccncc3)n2)co1] |
| B25 | 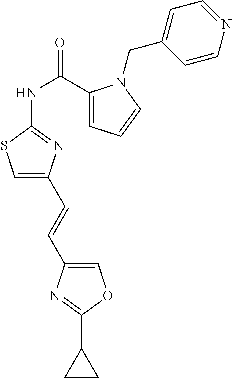 [SMILES: O=C(Nc1nc(/C=C/c2coc(C3CC3)n2)cs1)c1cccn1Cc1ccncc1] |
| B26 | 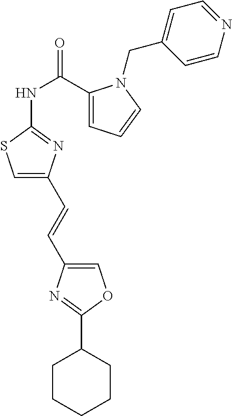 [SMILES: O=C(Nc1nc(/C=C/c2coc(C3CCCCC3)n2)cs1)c1cccn1Cc1ccncc1] |
| B27 | 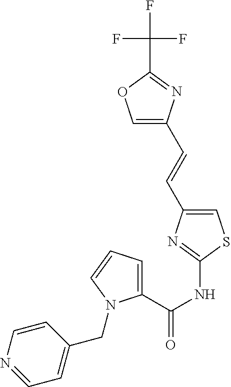 [SMILES: O=C(Nc1nc(/C=C/c2coc(C(F)(F)F)n2)cs1)c1cccn1Cc1ccncc1] |
| B28 | 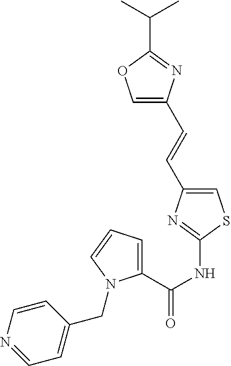 [SMILES: CC(C)c1nc(/C=C/c2csc(NC(=O)c3cccn3Cc3ccncc3)n2)co1] |
| B29 | 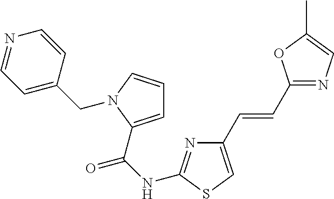 [SMILES: Cc1cnc(/C=C/c2csc(NC(=O)c3cccn3Cc3ccncc3)n2)o1] |
| B30 | 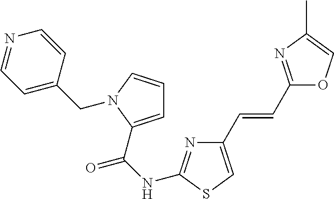 [SMILES: Cc1coc(/C=C/c2csc(NC(=O)c3cccn3Cc3ccncc3)n2)n1] |
| B31 | 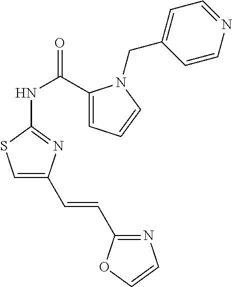 [SMILES: O=C(Nc1nc(/C=C/c2ncco2)cs1)c1cccn1Cc1ccncc1] |
| B32 | 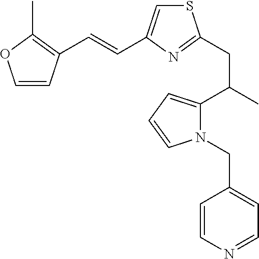 [SMILES: Cc1occc1/C=C/c1csc(CC(C)c2cccn2Cc2ccncc2)n1] |
| B33 | 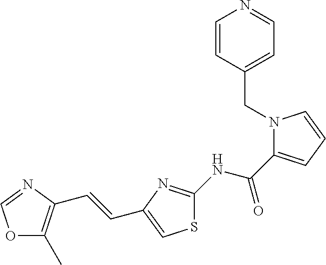 [SMILES: Cc1ocnc1/C=C/c1csc(NC(=O)c2cccn2Cc2ccncc2)n1] |
| B34 | 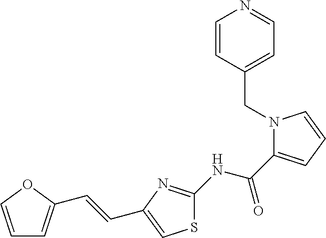 [SMILES: O=C(Nc1nc(/C=C/c2ccco2)cs1)c1cccn1Cc1ccncc1] |
| B35 | 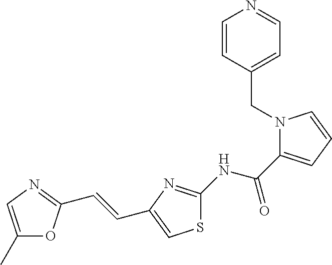 [SMILES: Cc1cnc(/C=C/c2csc(NC(=O)c3cccn3Cc3ccncc3)n2)o1] |
| B36 | 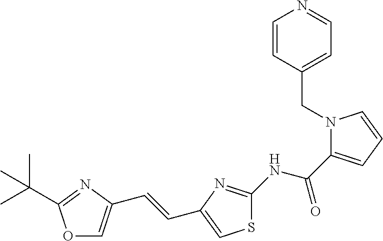 [SMILES: CC(C)(C)c1nc(/C=C/c2csc(NC(=O)c3cccn3Cc3ccncc3)n2)co1] |
| B37 | 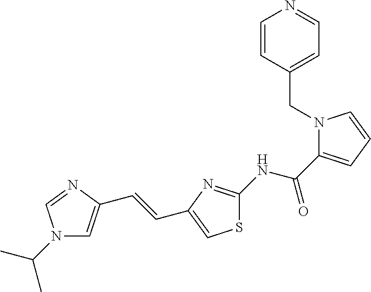 [SMILES: CC(C)n1cnc(/C=C/c2csc(NC(=O)c3cccn3Cc3ccncc3)n2)c1] |
| B38 | 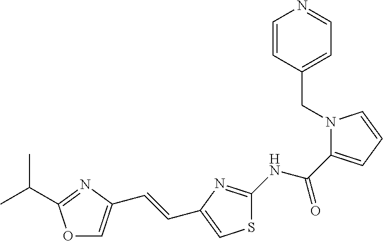 [SMILES: CC(C)c1nc(/C=C/c2csc(NC(=O)c3cccn3Cc3ccncc3)n2)co1] |
| B39 | 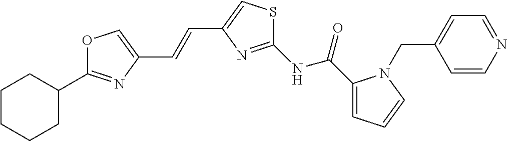 [SMILES: O=C(Nc1nc(/C=C/c2coc(C3CCCCC3)n2)cs1)c1cccn1Cc1ccncc1] |
| B40 | 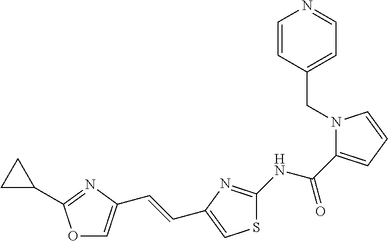 [SMILES: O=C(Nc1nc(/C=C/c2coc(C3CC3)n2)cs1)c1cccn1Cc1ccncc1] |
| B41 | 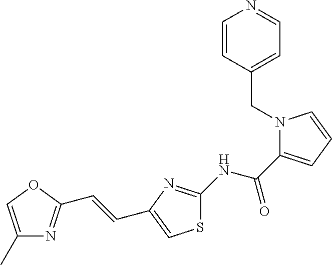 [SMILES: Cc1coc(/C=C/c2csc(NC(=O)c3cccn3Cc3ccncc3)n2)n1] |
| B42 | 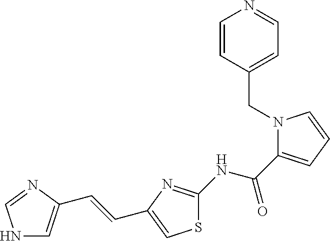 [SMILES: O=C(Nc1nc(/C=C/c2c[nH]cn2)cs1)c1cccn1Cc1ccncc1] |
| B43 | 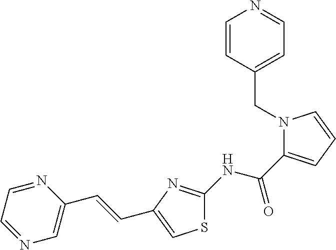 [SMILES: O=C(Nc1nc(/C=C/c2cnccn2)cs1)c1cccn1Cc1ccncc1] |
| B44 | 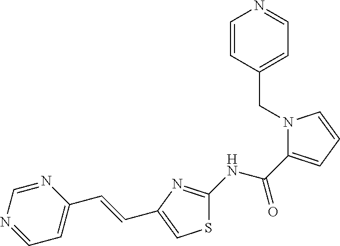 [SMILES: O=C(Nc1nc(/C=C/c2ccncn2)cs1)c1cccn1Cc1ccncc1] |
| B45 | 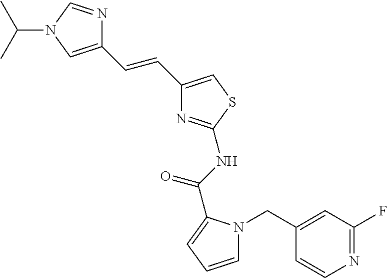 [SMILES: CC(C)n1cnc(/C=C/c2csc(NC(=O)c3cccn3Cc3ccnc(F)c3)n2)c1] |
| B47 | 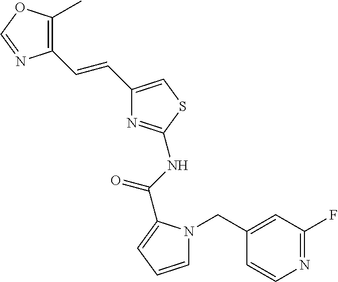 [SMILES: Cc1ocnc1/C=C/c1csc(NC(=O)c2cccn2Cc2ccnc(F)c2)n1] |
| B48 | 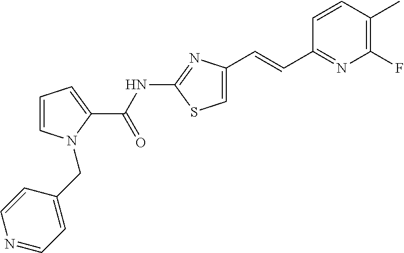 [SMILES: Cc1ccc(/C=C/c2csc(NC(=O)c3cccn3Cc3ccncc3)n2)nc1F] |
| B49 | 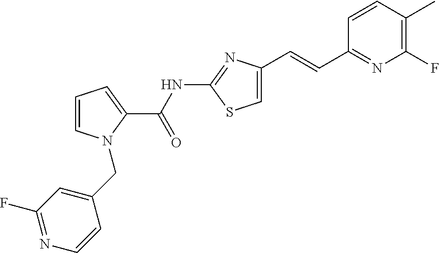 [SMILES: Cc1ccc(/C=C/c2csc(NC(=O)c3cccn3Cc3ccnc(F)c3)n2)nc1F] |
| B50 | 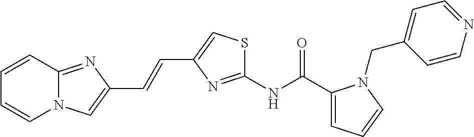 [SMILES: O=C(Nc1nc(/C=C/c2cn3ccccc3n2)cs1)c1cccn1Cc1ccncc1] |
| B51 | 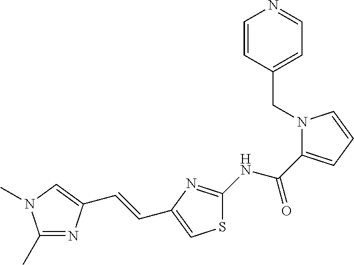 [SMILES: Cc1nc(/C=C/c2csc(NC(=O)c3cccn3Cc3ccncc3)n2)cn1C] |
| B52 | 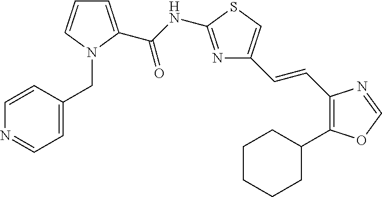 [SMILES: O=C(Nc1nc(/C=C/c2ncoc2C2CCCCC2)cs1)c1cccn1Cc1ccncc1] |
| B53 | 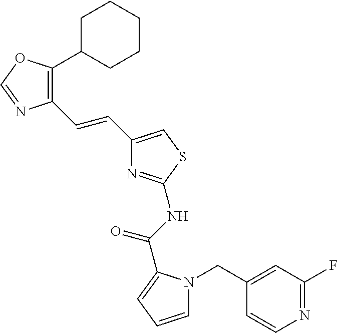 [SMILES: O=C(Nc1nc(/C=C/c2ncoc2C2CCCCC2)cs1)c1cccn1Cc1ccnc(F)c1] |
| B54 | 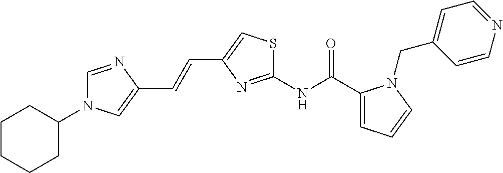 [SMILES: O=C(Nc1nc(/C=C/c2cn(C3CCCCC3)cn2)cs1)c1cccn1Cc1ccncc1] |
| B55 | 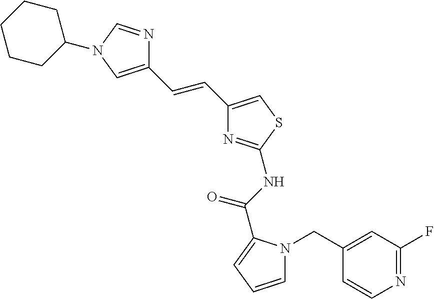 [SMILES: O=C(Nc1nc(/C=C/c2cn(C3CCCCC3)cn2)cs1)c1cccn1Cc1ccnc(F)c1] |
| B56 | 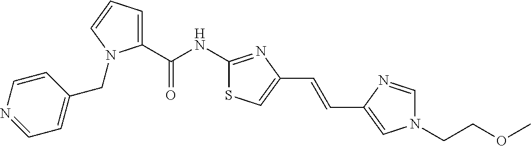 [SMILES: COCCn1cnc(/C=C/c2csc(NC(=O)c3cccn3Cc3ccncc3)n2)c1] |
| B57 | 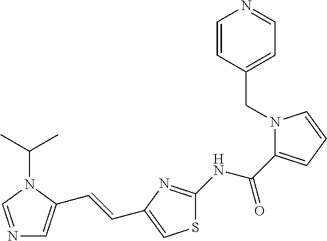 [SMILES: CC(C)n1cncc1/C=C/c1csc(NC(=O)c2cccn2Cc2ccncc2)n1] |
| B58 | 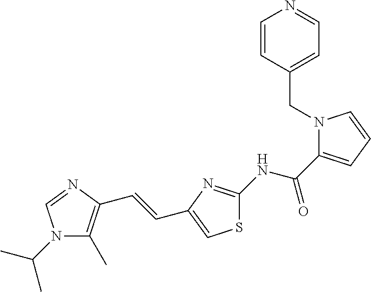 [SMILES: Cc1c(/C=C/c2csc(NC(=O)c3cccn3Cc3ccncc3)n2)ncn1C(C)C] |
| B59 | 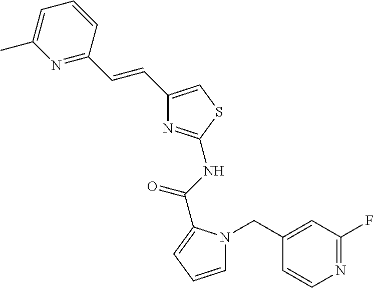 [SMILES: Cc1cccc(/C=C/c2csc(NC(=O)c3cccn3Cc3ccnc(F)c3)n2)n1] |
| B60 | 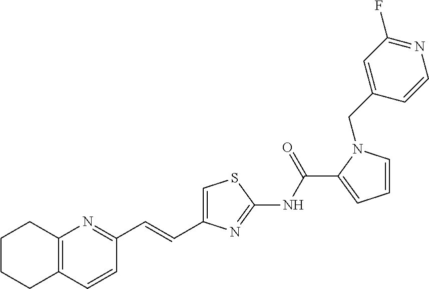 [SMILES: O=C(Nc1nc(/C=C/c2ccc3c(n2)CCCC3)cs1)c1cccn1Cc1ccnc(F)c1] |
| B61 | 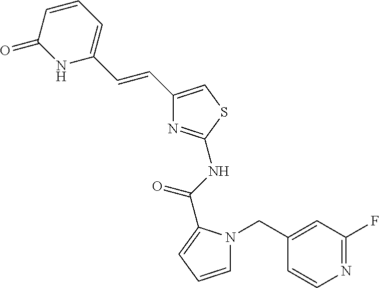 [SMILES: O=C(Nc1nc(/C=C/c2cccc(=O)[nH]2)cs1)c1cccn1Cc1ccnc(F)c1] |
| B62 | 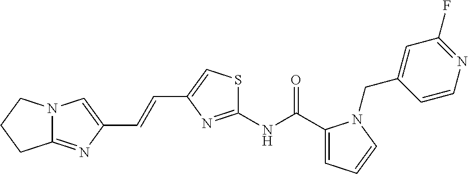 [SMILES: O=C(Nc1nc(/C=C/c2cn3c(n2)CCC3)cs1)c1cccn1Cc1ccnc(F)c1] |
| B63 | 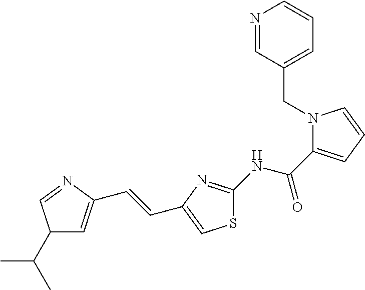 [SMILES: CC(C)C1C=NC(/C=C/c2csc(NC(=O)c3cccn3Cc3cccnc3)n2)=C1] |
| B64 | 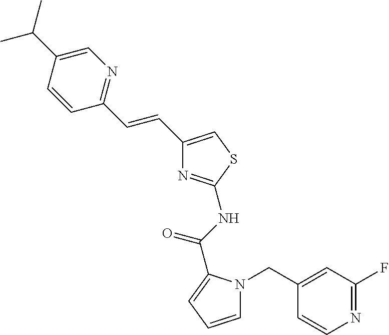 [SMILES: CC(C)c1ccc(/C=C/c2csc(NC(=O)c3cccn3Cc3ccnc(F)c3)n2)nc1] |
| B65 | 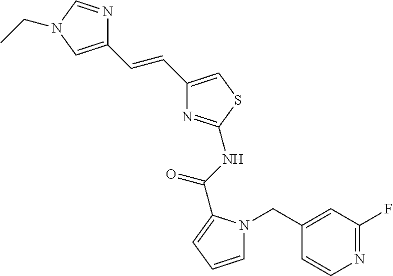 [SMILES: CCn1cnc(/C=C/c2csc(NC(=O)c3cccn3Cc3ccnc(F)c3)n2)c1] |
| B66 | 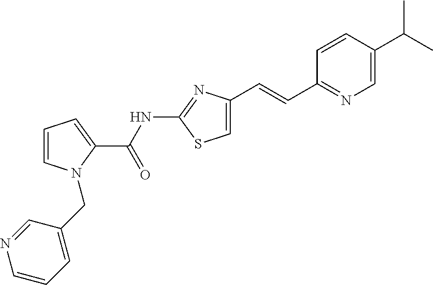 [SMILES: CC(C)c1ccc(/C=C/c2csc(NC(=O)c3cccn3Cc3cccnc3)n2)nc1] |
| B67 | 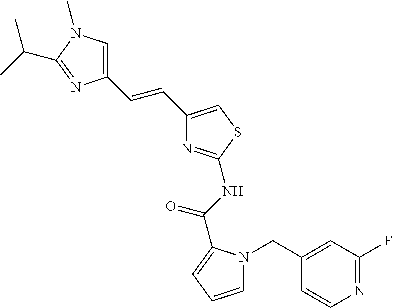 [SMILES: CC(C)c1nc(/C=C/c2csc(NC(=O)c3cccn3Cc3ccnc(F)c3)n2)cn1C] |
| B68 | 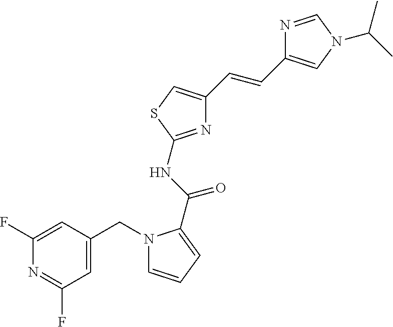 [SMILES: CC(C)n1cnc(/C=C/c2csc(NC(=O)c3cccn3Cc3cc(F)nc(F)c3)n2)c1] |
| B69 | 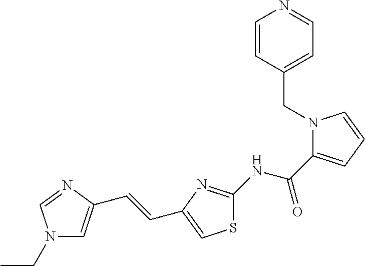 [SMILES: CCn1cnc(/C=C/c2csc(NC(=O)c3cccn3Cc3ccncc3)n2)c1] |
| B70 | 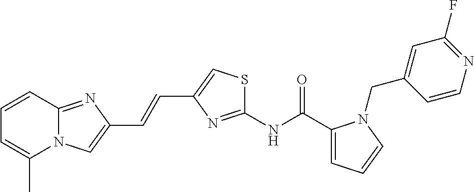 [SMILES: Cc1cccc2nc(/C=C/c3csc(NC(=O)c4cccn4Cc4ccnc(F)c4)n3)cn12] |
| B71 | 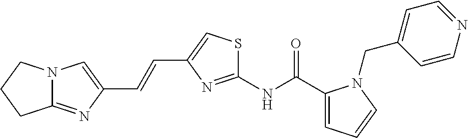 [SMILES: O=C(Nc1nc(/C=C/c2cn3c(n2)CCC3)cs1)c1cccn1Cc1ccncc1] |
| B72 | 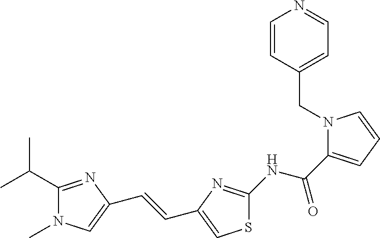 [SMILES: CC(C)c1nc(/C=C/c2csc(NC(=O)c3cccn3Cc3ccncc3)n2)cn1C] |
| B73 | 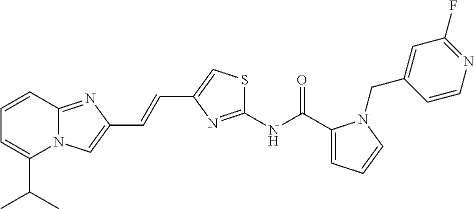 [SMILES: CC(C)c1cccc2nc(/C=C/c3csc(NC(=O)c4cccn4Cc4ccnc(F)c4)n3)cn12] |
| B74 | 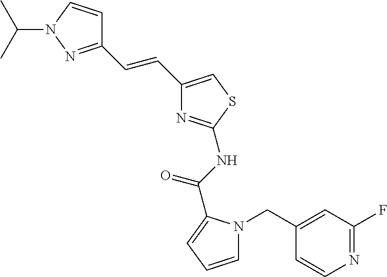 [SMILES: CC(C)n1ccc(/C=C/c2csc(NC(=O)c3cccn3Cc3ccnc(F)c3)n2)n1] |
| B75 | 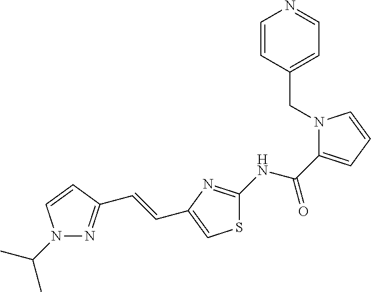 [SMILES: CC(C)n1ccc(/C=C/c2csc(NC(=O)c3cccn3Cc3ccncc3)n2)n1] |
| B76 | 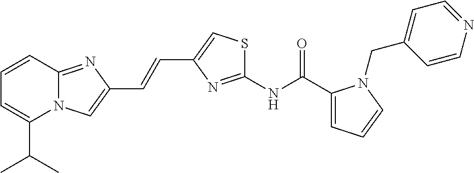 [SMILES: CC(C)c1cccc2nc(/C=C/c3csc(NC(=O)c4cccn4Cc4ccncc4)n3)cn12] |
| B77 | 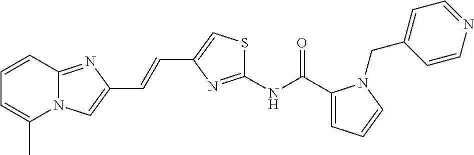 [SMILES: Cc1cccc2nc(/C=C/c3csc(NC(=O)c4cccn4Cc4ccncc4)n3)cn12] |
| B78 | 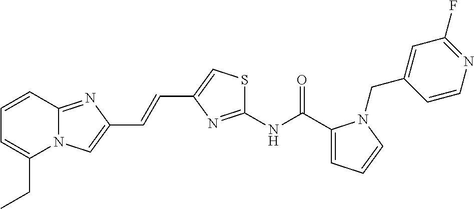 [SMILES: CCc1cccc2nc(/C=C/c3csc(NC(=O)c4cccn4Cc4ccnc(F)c4)n3)cn12] |
| B79 | 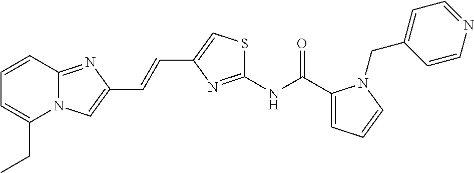 [SMILES: CCc1cccc2nc(/C=C/c3csc(NC(=O)c4cccn4Cc4ccncc4)n3)cn12] |
| B80 | 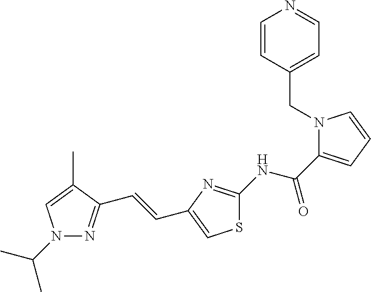 [SMILES: Cc1cn(C(C)C)nc1/C=C/c1csc(NC(=O)c2cccn2Cc2ccncc2)n1] |
| B81 | 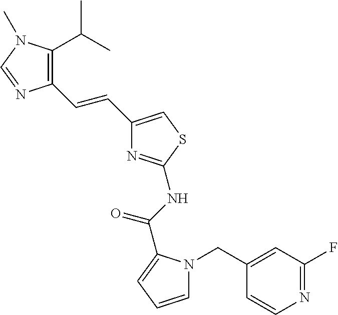 [SMILES: CC(C)c1c(/C=C/c2csc(NC(=O)c3cccn3Cc3ccnc(F)c3)n2)ncn1C] |
| B83 | 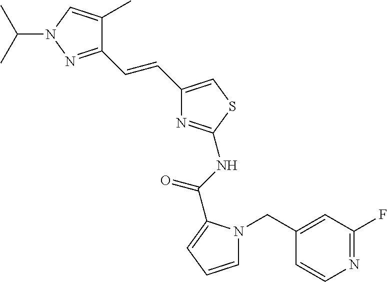 [SMILES: Cc1cn(C(C)C)nc1/C=C/c1csc(NC(=O)c2cccn2Cc2ccnc(F)c2)n1] |
| B84 | 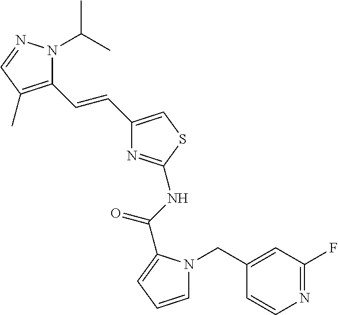 [SMILES: Cc1cnn(C(C)C)c1/C=C/c1csc(NC(=O)c2cccn2Cc2ccnc(F)c2)n1] |
| B85 | 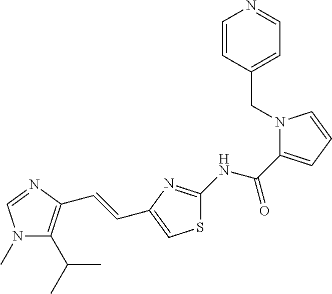 [SMILES: CC(C)c1c(/C=C/c2csc(NC(=O)c3cccn3Cc3ccncc3)n2)ncn1C] |
| B86 | 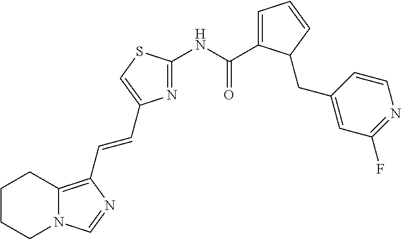 [SMILES: O=C(Nc1nc(/C=C/c2ncn3c2CCCC3)cs1)C1=CC=CC1Cc1ccnc(F)c1] |
| B87 | 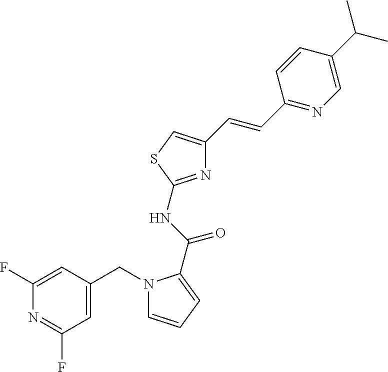 [SMILES: CC(C)c1ccc(/C=C/c2csc(NC(=O)c3cccn3Cc3cc(F)nc(F)c3)n2)nc1] |
| B88 | 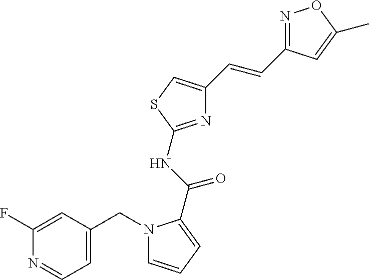 [SMILES: Cc1cc(/C=C/c2csc(NC(=O)c3cccn3Cc3ccnc(F)c3)n2)no1] |
| B89 | 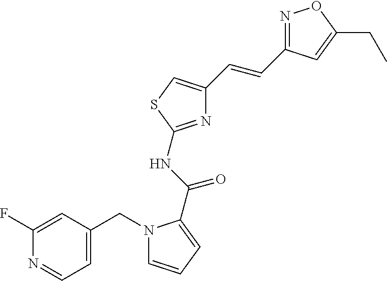 [SMILES: CCc1cc(/C=C/c2csc(NC(=O)c3cccn3Cc3ccnc(F)c3)n2)no1] |
| B90 | 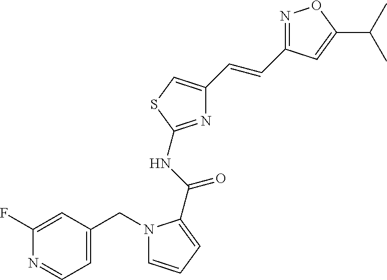 [SMILES: CC(C)c1cc(/C=C/c2csc(NC(=O)c3cccn3Cc3ccnc(F)c3)n2)no1] |
| B91 | 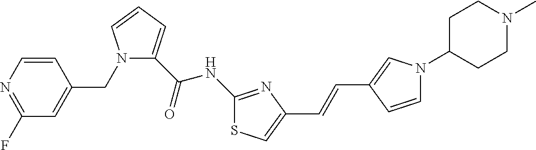 [SMILES: CN1CCC(n2ccc(/C=C/c3csc(NC(=O)c4cccn4Cc4ccnc(F)c4)n3)c2)CC1] |
| B92 | 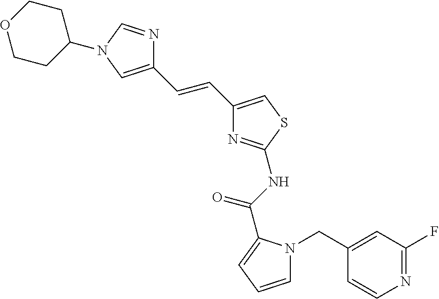 [SMILES: O=C(Nc1nc(/C=C/c2cn(C3CCOCC3)cn2)cs1)c1cccn1Cc1ccnc(F)c1] |
| B93 | 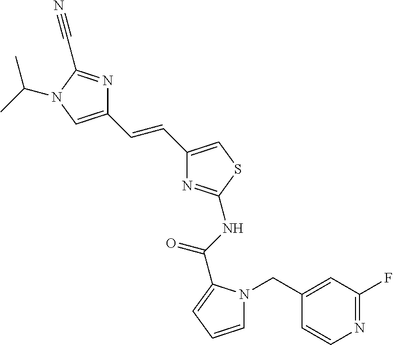 [SMILES: CC(C)n1cc(/C=C/c2csc(NC(=O)c3cccn3Cc3ccnc(F)c3)n2)nc1C#N] |
| B94 | 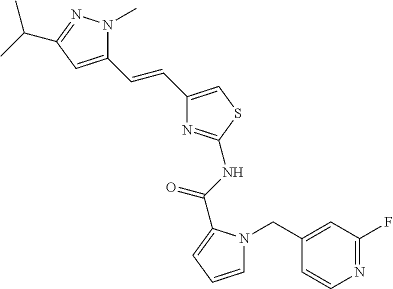 [SMILES: CC(C)c1cc(/C=C/c2csc(NC(=O)c3cccn3Cc3ccnc(F)c3)n2)n(C)n1] |
| B95 | 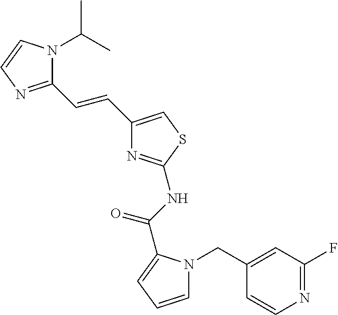 [SMILES: CC(C)n1ccnc1/C=C/c1csc(NC(=O)c2cccn2Cc2ccnc(F)c2)n1] |
| B96 | 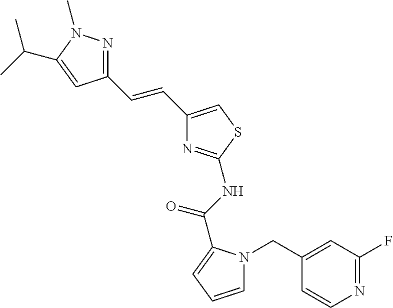 [SMILES: CC(C)c1cc(/C=C/c2csc(NC(=O)c3cccn3Cc3ccnc(F)c3)n2)nn1C] |
| B97 | 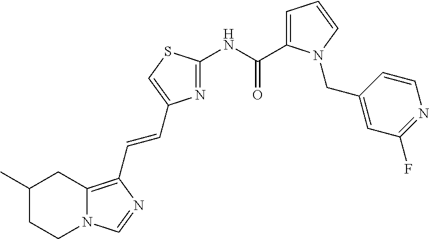 [SMILES: CC1CCn2cnc(/C=C/c3csc(NC(=O)c4cccn4Cc4ccnc(F)c4)n3)c2C1] |
| B98 | 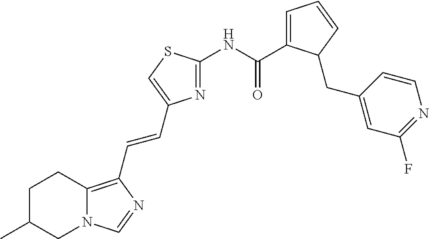 [SMILES: CC1CCc2c(/C=C/c3csc(NC(=O)C4=CC=CC4Cc4ccnc(F)c4)n3)ncn2C1] |
| B99 | 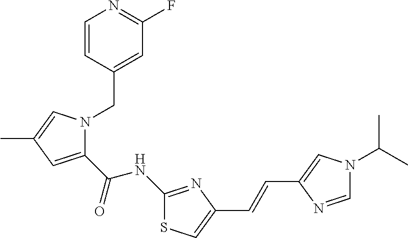 [SMILES: Cc1cc(C(=O)Nc2nc(/C=C/c3cn(C(C)C)cn3)cs2)n(Cc2ccnc(F)c2)c1] |
| B100 | 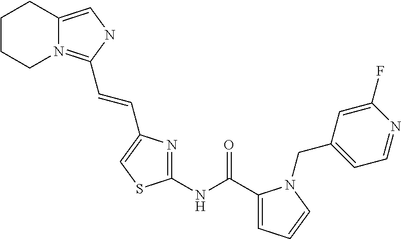 |
| B101 | 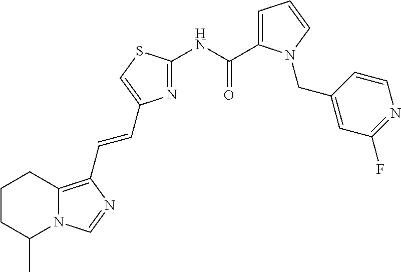 [SMILES: CC1CCCc2c(/C=C/c3csc(NC(=O)c4cccn4Cc4ccnc(F)c4)n3)ncn21] |
| B102 | 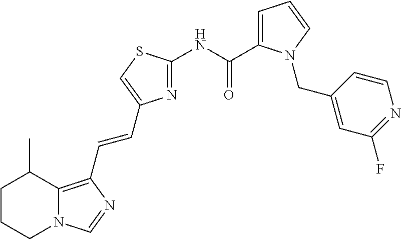 [SMILES: CC1CCCn2cnc(/C=C/c3csc(NC(=O)c4cccn4Cc4ccnc(F)c4)n3)c21] |
| B103 | 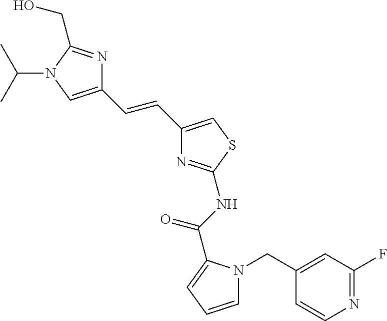 [SMILES: CC(C)n1cc(/C=C/c2csc(NC(=O)c3cccn3Cc3ccnc(F)c3)n2)nc1CO] |
| B104 | 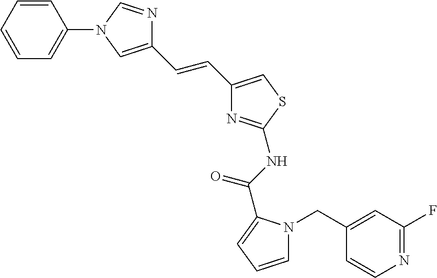 [SMILES: O=C(Nc1nc(/C=C/c2cn(-c3ccccc3)cn2)cs1)c1cccn1Cc1ccnc(F)c1] |
| B105 | 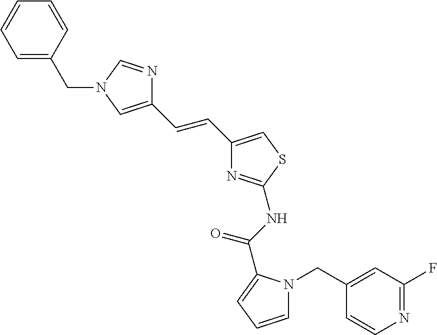 [SMILES: O=C(Nc1nc(/C=C/c2cn(Cc3ccccc3)cn2)cs1)c1cccn1Cc1ccnc(F)c1] |
| B106 | 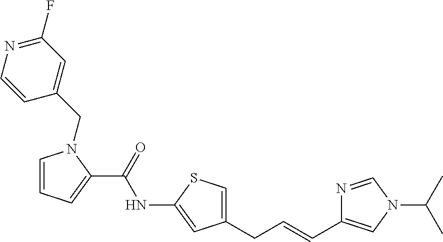 [SMILES: CC(C)n1cnc(/C=C/Cc2csc(NC(=O)c3cccn3Cc3ccnc(F)c3)c2)c1] |
| B107 | 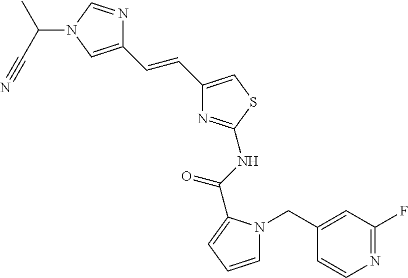 [SMILES: CC(C#N)n1cnc(/C=C/c2csc(NC(=O)c3cccn3Cc3ccnc(F)c3)n2)c1] |
| B108 | 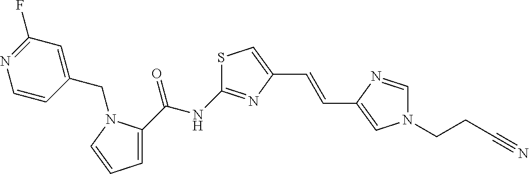 [SMILES: N#CCCn1cnc(/C=C/c2csc(NC(=O)c3cccn3Cc3ccnc(F)c3)n2)c1] |
| B109 | 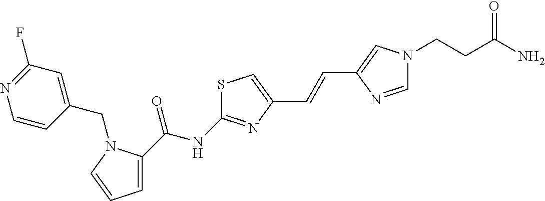 [SMILES: NC(=O)CCn1cnc(/C=C/c2csc(NC(=O)c3cccn3Cc3ccnc(F)c3)n2)c1] |
| B110 | 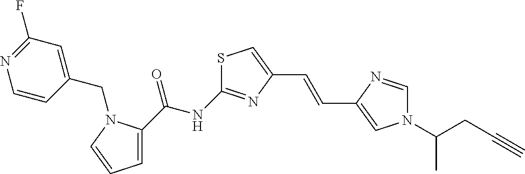 [SMILES: C#CCC(C)n1cnc(/C=C/c2csc(NC(=O)c3cccn3Cc3ccnc(F)c3)n2)c1] |
| B111 | 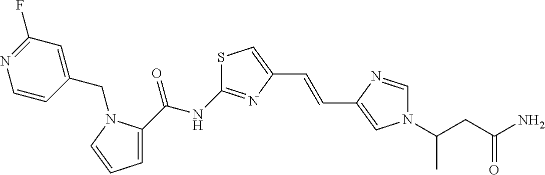 [SMILES: CC(CC(N)=O)n1cnc(/C=C/c2csc(NC(=O)c3cccn3Cc3ccnc(F)c3)n2)c1] |
| B112 | 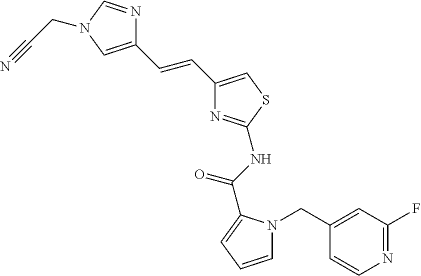 [SMILES: N#CCn1cnc(/C=C/c2csc(NC(=O)c3cccn3Cc3ccnc(F)c3)n2)c1] |
| B113 | 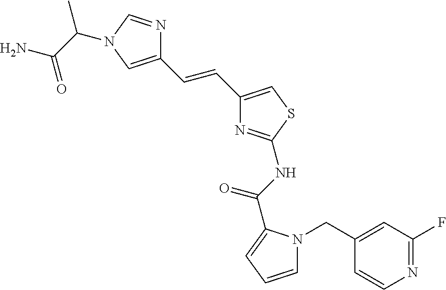 [SMILES: CC(C(N)=O)n1cnc(/C=C/c2csc(NC(=O)c3cccn3Cc3ccnc(F)c3)n2)c1] |
| B114 | 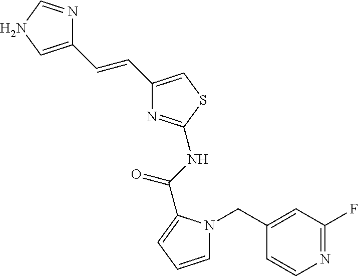 [SMILES: O=C(Nc1nc(/C=C/c2c[nH]cn2)cs1)c1cccn1Cc1ccnc(F)c1] |
| B115 | 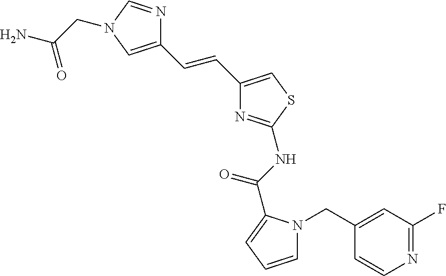 [SMILES: NC(=O)Cn1cnc(/C=C/c2csc(NC(=O)c3cccn3Cc3ccnc(F)c3)n2)c1] |
| B116 | 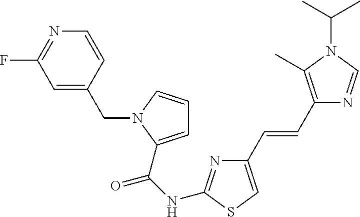 [SMILES: Cc1c(/C=C/c2csc(NC(=O)c3cccn3Cc3ccnc(F)c3)n2)ncn1C(C)C] |
| B117 | 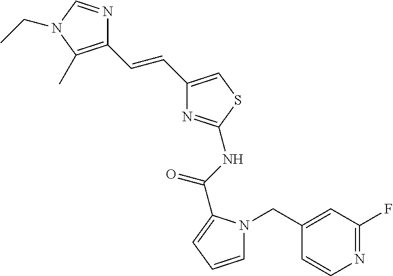 [SMILES: CCn1cnc(/C=C/c2csc(NC(=O)c3cccn3Cc3ccnc(F)c3)n2)c1C] |
| B118 | 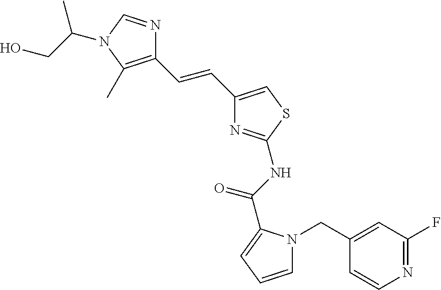 [SMILES: Cc1c(/C=C/c2csc(NC(=O)c3cccn3Cc3ccnc(F)c3)n2)ncn1C(C)CO] |
| B119 | 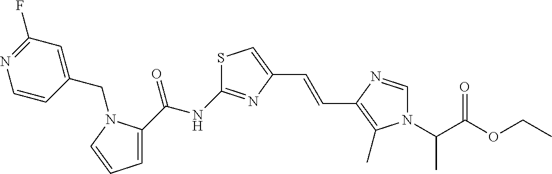 [SMILES: CCOC(=O)C(C)n1cnc(/C=C/c2csc(NC(=O)c3cccn3Cc3ccnc(F)c3)n2)c1C] |
| B120 | 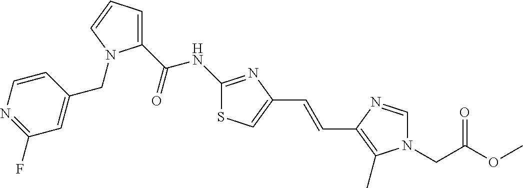 [SMILES: COC(=O)Cn1cnc(/C=C/c2csc(NC(=O)c3cccn3Cc3ccnc(F)c3)n2)c1C] |
| B121 | 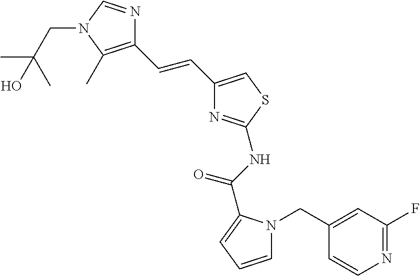 [SMILES: Cc1c(/C=C/c2csc(NC(=O)c3cccn3Cc3ccnc(F)c3)n2)ncn1CC(C)(C)O] |
| B122 | 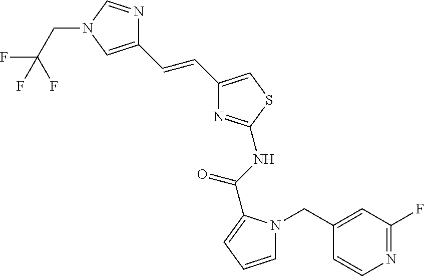 [SMILES: O=C(Nc1nc(/C=C/c2cn(CC(F)(F)F)cn2)cs1)c1cccn1Cc1ccnc(F)c1] |
| B123 | 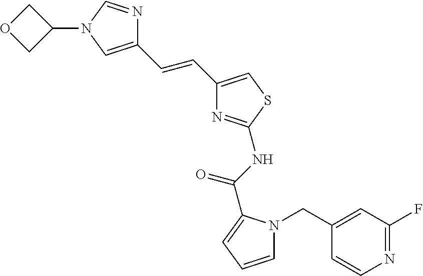 [SMILES: O=C(Nc1nc(/C=C/c2cn(C3COC3)cn2)cs1)c1cccn1Cc1ccnc(F)c1] |
| B124 | 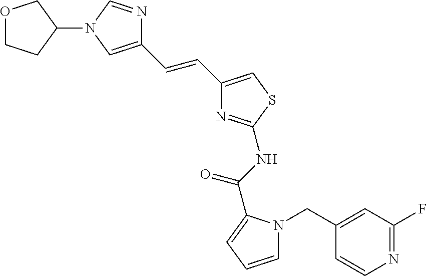 [SMILES: O=C(Nc1nc(/C=C/c2cn(C3CCOC3)cn2)cs1)c1cccn1Cc1ccnc(F)c1] |
| B125 | 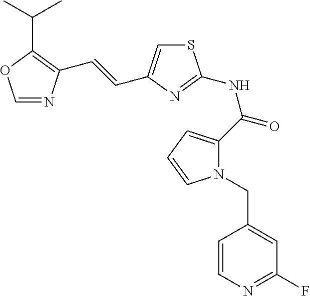 [SMILES: CC(C)c1ocnc1/C=C/c1csc(NC(=O)c2cccn2Cc2ccnc(F)c2)n1] |
| B126 | 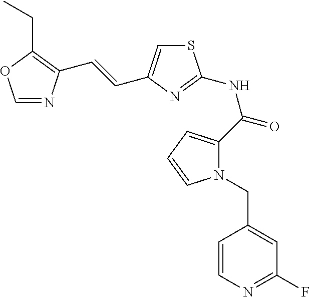 [SMILES: CCc1ocnc1/C=C/c1csc(NC(=O)c2cccn2Cc2ccnc(F)c2)n1] |
| B127 | 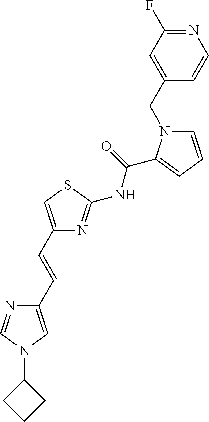 [SMILES: O=C(Nc1nc(/C=C/c2cn(C3CCC3)cn2)cs1)c1cccn1Cc1ccnc(F)c1] |
| B128 | 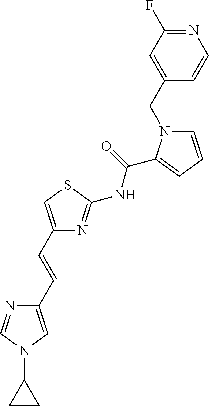 [SMILES: O=C(Nc1nc(/C=C/c2cn(C3CC3)cn2)cs1)c1cccn1Cc1ccnc(F)c1] |
| B129 | 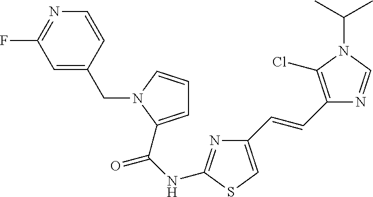 [SMILES: CC(C)n1cnc(/C=C/c2csc(NC(=O)c3cccn3Cc3ccnc(F)c3)n2)c1Cl] |
| B130 | 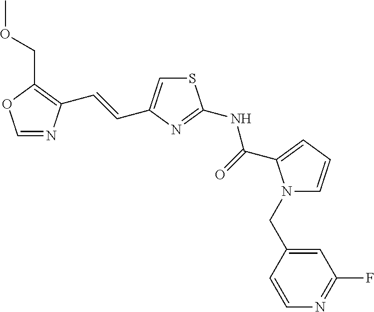 [SMILES: COCc1ocnc1/C=C/c1csc(NC(=O)c2cccn2Cc2ccnc(F)c2)n1] |
| B131 | 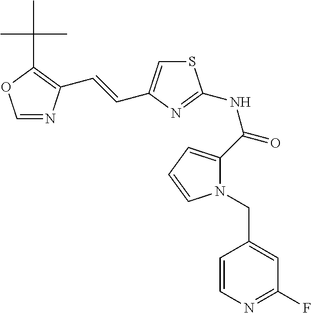 [SMILES: CC(C)(C)c1ocnc1/C=C/c1csc(NC(=O)c2cccn2Cc2ccnc(F)c2)n1] |
| B132 | 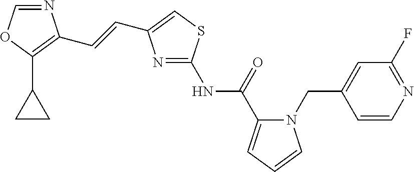 [SMILES: O=C(Nc1nc(/C=C/c2ncoc2C2CC2)cs1)c1cccn1Cc1ccnc(F)c1] |
| B133 | 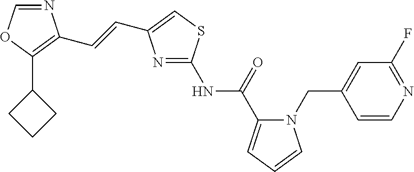 [SMILES: O=C(Nc1nc(/C=C/c2ncoc2C2CCC2)cs1)c1cccn1Cc1ccnc(F)c1] |
| B134 | 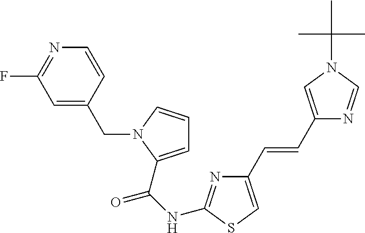 [SMILES: CC(C)(C)n1cnc(/C=C/c2csc(NC(=O)c3cccn3Cc3ccnc(F)c3)n2)c1] |
| B135 | 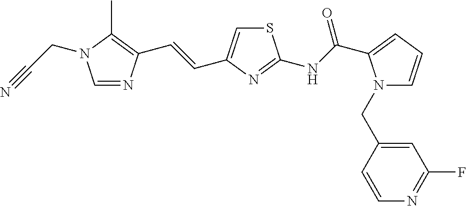 [SMILES: Cc1c(/C=C/c2csc(NC(=O)c3cccn3Cc3ccnc(F)c3)n2)ncn1CC#N] |
| B136 | 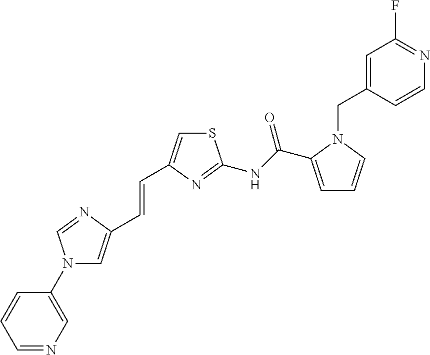 [SMILES: O=C(Nc1nc(/C=C/c2cn(-c3cccnc3)cn2)cs1)c1cccn1Cc1ccnc(F)c1] |
| B137 | 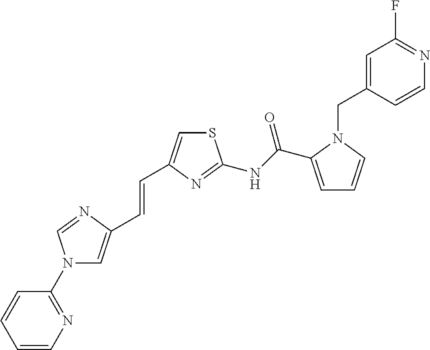 [SMILES: O=C(Nc1nc(/C=C/c2cn(-c3ccccn3)cn2)cs1)c1cccn1Cc1ccnc(F)c1] |
| B138 | 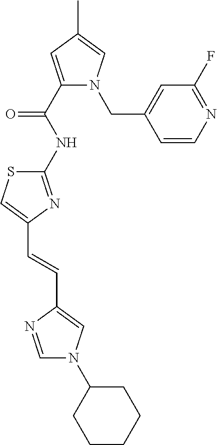 [SMILES: Cc1cc(C(=O)Nc2nc(/C=C/c3cn(C4CCCCC4)cn3)cs2)n(Cc2ccnc(F)c2)c1] |
| B139 | 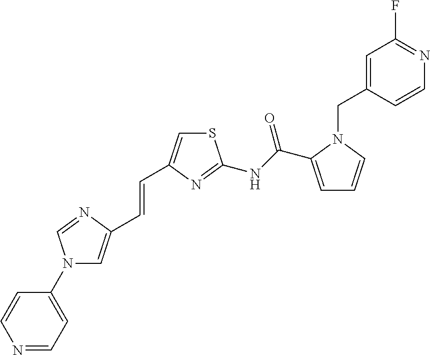 [SMILES: O=C(Nc1nc(/C=C/c2cn(-c3ccncc3)cn2)cs1)c1cccn1Cc1ccnc(F)c1] |
| B141 | 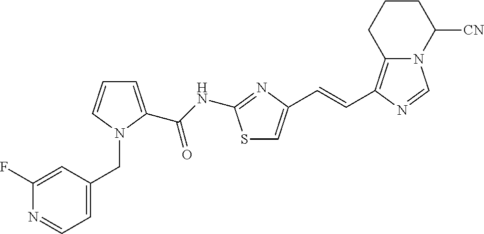 [SMILES: N#CC1CCCc2c(/C=C/c3csc(NC(=O)c4cccn4Cc4ccnc(F)c4)n3)ncn21] |
| B142 | 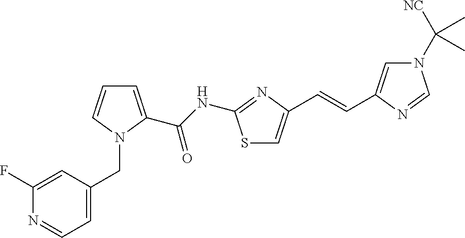 [SMILES: CC(C)(C#N)n1cnc(/C=C/c2csc(NC(=O)c3cccn3Cc3ccnc(F)c3)n2)c1] |
| B143 | 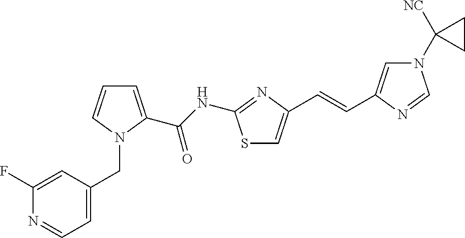 [SMILES: N#CC1(n2cnc(/C=C/c3csc(NC(=O)c4cccn4Cc4ccnc(F)c4)n3)c2)CC1] |
| B144 | 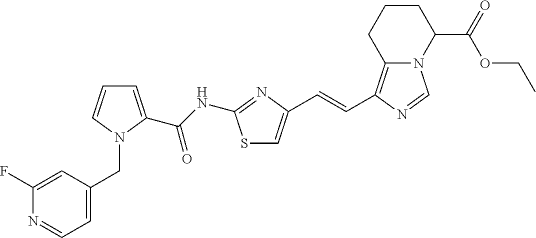 [SMILES: CCOC(=O)C1CCCc2c(/C=C/c3csc(NC(=O)c4cccn4Cc4ccnc(F)c4)n3)ncn21] |
| B145 | 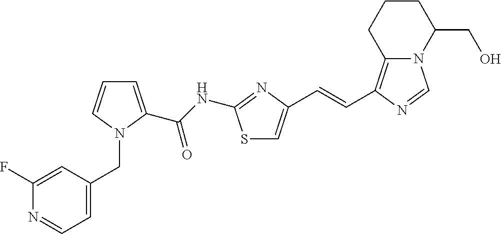 [SMILES: O=C(Nc1nc(/C=C/c2ncn3c2CCCC3CO)cs1)c1cccn1Cc1ccnc(F)c1] |
| B146 | 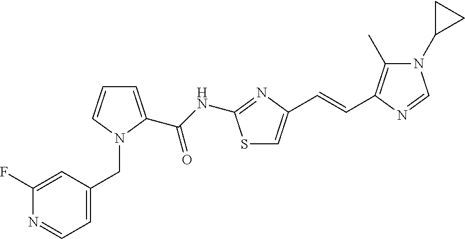 [SMILES: Cc1c(/C=C/c2csc(NC(=O)c3cccn3Cc3ccnc(F)c3)n2)ncn1C1CC1] |
| B147 | 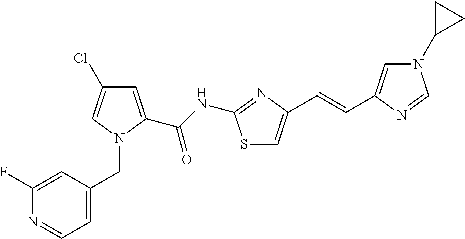 [SMILES: O=C(Nc1nc(/C=C/c2cn(C3CC3)cn2)cs1)c1cc(Cl)cn1Cc1ccnc(F)c1] |
| B148 | 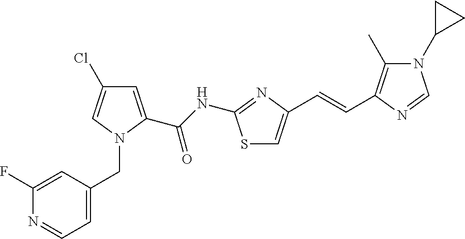 [SMILES: Cc1c(/C=C/c2csc(NC(=O)c3cc(Cl)cn3Cc3ccnc(F)c3)n2)ncn1C1CC1] |
| B149 | 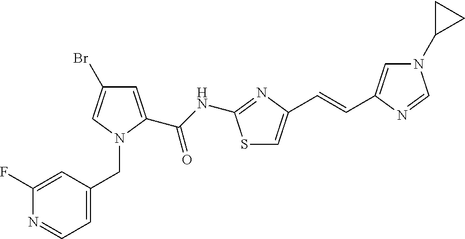 [SMILES: O=C(Nc1nc(/C=C/c2cn(C3CC3)cn2)cs1)c1cc(Br)cn1Cc1ccnc(F)c1] |
| B150 | 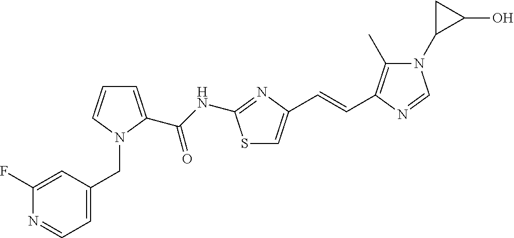 [SMILES: Cc1c(/C=C/c2csc(NC(=O)c3cccn3Cc3ccnc(F)c3)n2)ncn1C1CC1O] |
| B151 | 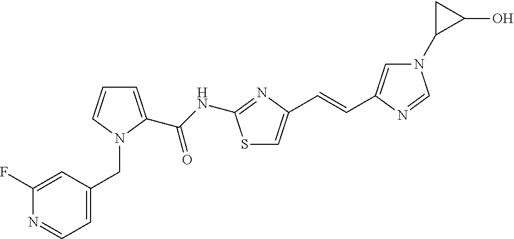 [SMILES: O=C(Nc1nc(/C=C/c2cn(C3CC3O)cn2)cs1)c1cccn1Cc1ccnc(F)c1] |
| B152 | 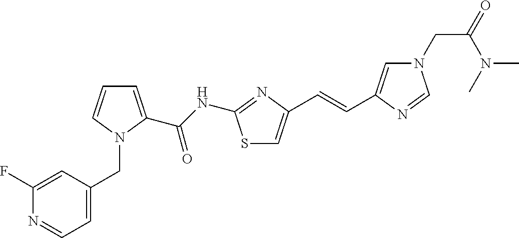 [SMILES: CN(C)C(=O)Cn1cnc(/C=C/c2csc(NC(=O)c3cccn3Cc3ccnc(F)c3)n2)c1] |
| B153 | 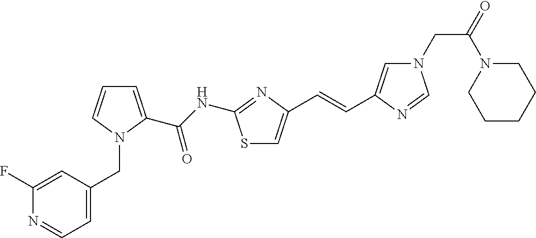 [SMILES: O=C(Nc1nc(/C=C/c2cn(CC(=O)N3CCCCC3)cn2)cs1)c1cccn1Cc1ccnc(F)c1] |
| B154 | 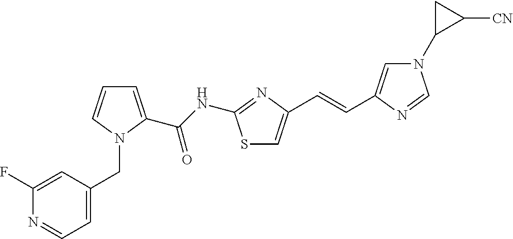 [SMILES: N#CC1CC1n1cnc(/C=C/c2csc(NC(=O)c3cccn3Cc3ccnc(F)c3)n2)c1] |
| B155 | 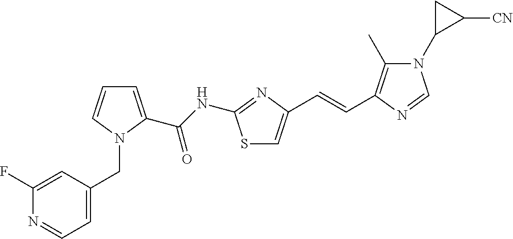 [SMILES: Cc1c(/C=C/c2csc(NC(=O)c3cccn3Cc3ccnc(F)c3)n2)ncn1C1CC1C#N] |
| B156 | 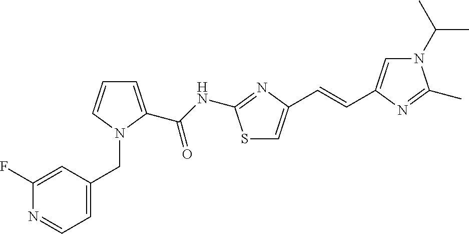 [SMILES: Cc1nc(/C=C/c2csc(NC(=O)c3cccn3Cc3ccnc(F)c3)n2)cn1C(C)C] |
| B157 | 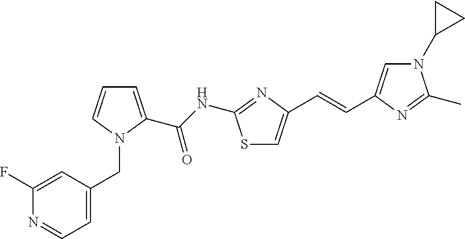 [SMILES: Cc1nc(/C=C/c2csc(NC(=O)c3cccn3Cc3ccnc(F)c3)n2)cn1C1CC1] |
| B158 | 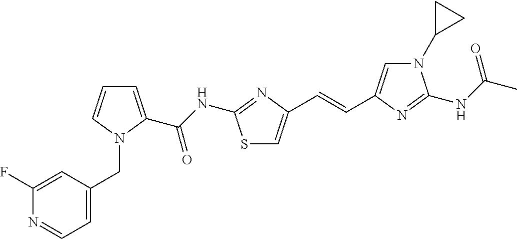 [SMILES: CC(=O)Nc1nc(/C=C/c2csc(NC(=O)c3cccn3Cc3ccnc(F)c3)n2)cn1C1CC1] |
| B159 | 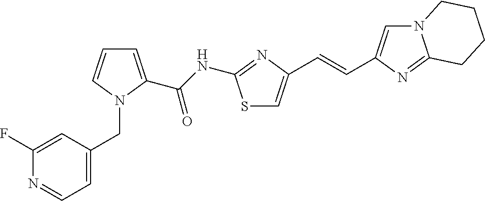 [SMILES: O=C(Nc1nc(/C=C/c2cn3c(n2)CCCC3)cs1)c1cccn1Cc1ccnc(F)c1] |
| B160 | 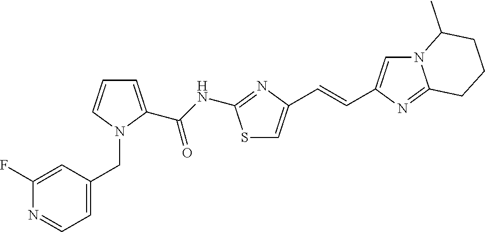 [SMILES: CC1CCCc2nc(/C=C/c3csc(NC(=O)c4cccn4Cc4ccnc(F)c4)n3)cn21] |
| E1 | 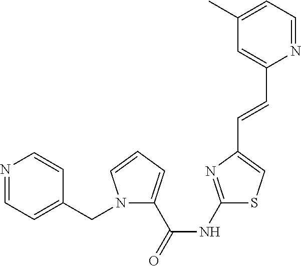 [SMILES: Cc1ccnc(/C=C/c2csc(NC(=O)c3cccn3Cc3ccncc3)n2)c1] |
| E55 | 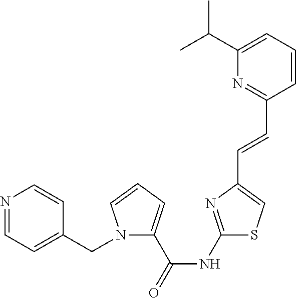 [SMILES: CC(C)c1cccc(/C=C/c2csc(NC(=O)c3cccn3Cc3ccncc3)n2)n1] |
| E56 | 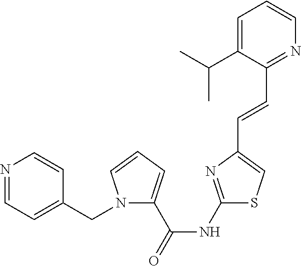 [SMILES: CC(C)c1cccnc1/C=C/c1csc(NC(=O)c2cccn2Cc2ccncc2)n1] |
| E57 | 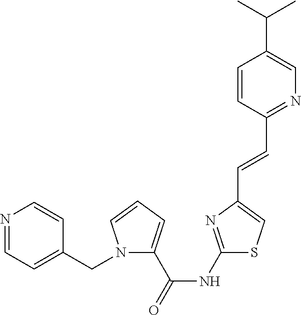 [SMILES: CC(C)c1ccc(/C=C/c2csc(NC(=O)c3cccn3Cc3ccncc3)n2)nc1] |

### Compounds of Formula (III)

Further provided herein are compounds, or pharmaceutically acceptable salts thereof, having a structure of formula (III):

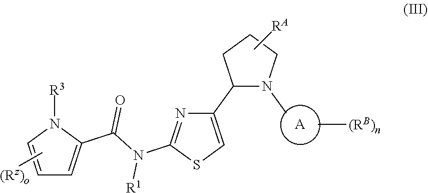

wherein
R¹ is H, C₁₋₃alkyl, or SO₂C₁₋₆alkyl;
R^{A} is H, C₁₋₆alkyl, OR^{N}, N(R^{N})₂, OC₁₋₆alkylene-N(R^{N})₂, or OC₁₋₆alkylene-OR^{N};
n is 0, 1, or 2;
ring A is phenyl or a 6-membered heteroaryl having 1 or 2 nitrogen ring atoms;
each R^{B}, when present, is independently C₁₋₆alkyl, C₁₋₆alkoxy, C₁₋₆haloalkoxy, C₁₋₃alkylene-C₁₋₃alkoxy, C₁₋₆haloalkyl, C₁₋₆hydroxyalkyl, halo, C₀₋₃alkylene-CO₂R^{N}, C₀₋₃alkylene-C(O)N(R^{N})₂, C₀₋₃alkylene-N(R^{N})₂, OC₁₋₃alkylene-N(R^{N})₂, NO₂, C₀₋₃alkylene-N(R^{N})C(O)R^{N}, C₀₋₃alkylene-N(R^{N})C(O)OR^{N}, OC₁₋₃alkylene-N(R^{N})C(O)R^{N}, C₀₋₃alkylene-N(R^{N})C(O)N(R^{N})₂, C₀₋₃alkylene-N(R^{N})SO₂R^{N}, C₀₋₃alkylene-OC(O)N(R^{N})₂, C₀₋₃alkylene-Het, C₀₋₃alkylene-OHet, C₀₋₃alkylene-NHCO₂Het, C₀₋₃alkylene-OC(O)Het, C₀₋₃alkylene-N(R^{N})Het or C₀₋₃alkylene-N(R^{N})C(O)Het;
Het is an aromatic or non-aromatic 4-7 membered ring having 0-3 ring heteroatoms selected from N, O, and S;
Het is optionally substituted with 1 substituent selected from C₁₋₆alkyl, OR^{N}, halo, oxo, C(O)R^{N}, C(O)N(R^{N})₂, SOR^{N}, SO₂N(R^{N})₂, and SO₂R^{N};
R³ is C₁₋₆alkylene-X, C₂₋₆alkenylene-X, C₀₋₂alkylene-C₃₋₆carbocycle-C₀₋₂alkylene-X, or Ar, and the alkylene is optionally substituted with OR^{N}
X is H, OC₁₋₃alkyl, C≡CR^{N}; CN, CO₂R^{N}; CON(R^{N})₂, or Ar,
Ar is a 3-10 membered aromatic or non-aromatic monocyclic or polycyclic ring having 0-4 ring heteroatoms selected from N, O, and S, with the proviso that when Ar is a 6-membered aromatic ring, it has 0 or 2-4 ring heteroatoms,
Ar is optionally substituted with C₁₋₃alkyl, C₀₋₂alkylene-CN, CON(R^{N})₂, tetrazole, oxazole, or 1-2 halo;
o is 0 or 1;
R^{z}, when present, is CN, halo, C(O)N(R^{N})₂, C₁₋₆alkyl, C₁₋₆alkoxy, C₁₋₆hydroxyalkyl, or C₁₋₆haloalkyl; and
each R^{N} is independently H, C₁₋₆alkyl, C₁₋₆hydroxyalkyl, or C₁₋₆haloalkyl.

In various cases, R¹ is H. In various cases, R^{A} is H. In some cases, R^{A} is OC₁₋₆alkylene-N(R^{N})₂ or OC₁₋₆ alkylene-OR^{N}. In some cases, R^{A} is OR^{N} or N(R^{N})₂. In various cases, each R^{N} is H or methyl. In some cases, at least one R^{N} is C₁₋₆hydroxyalkyl or C₁₋₆haloalkyl.

In various cases, ring A is phenyl. In various cases, ring A is pyridyl. In various cases, ring A is a diazinyl-pyrimidinyl or pyrazinyl or pyradazinyl. In various cases, ring A is unsubstituted (i.e., n is 0). In various cases, ring A is substituted (i.e., n is 1 or 2). In some cases, n is 1. The substitution(s) —R^{B}— can be C₁₋₆alkyl, C₁₋₆alkoxy, C₁₋₆haloalkoxy, C₁₋₃alkylene-C₁₋₃alkoxy, C₁₋₆haloalkyl, C₁₋₆hydroxyalkyl, halo, C₃₋₆cycloalkyl, CO₂R^{N}, C₀₋₃alkylene-C(O)N(R^{N})₂, N(R^{N})₂, NO₂, C₀₋₃alkylene-N(R^{N})C(O)R^{N}, C₀₋₃alkylene-N(R^{N})C(O)R^{N}, Het, or OHet. In some cases, R^{B} is C₁₋₆alkyl. In some cases, R^{B} is C₁₋₆haloalkyl, C₁₋₆hydroxyalkyl, or halo. In some cases, R^{B} is CO₂R^{N}, N(R^{N})₂, C₀₋₃alkylene-C(O)N(R^{N})₂, or C₀₋₃alkylene-N(R^{N})C(O)R^{N}. In some cases, R^{B} is C₃₋₆cycloalkyl, Het, or OHet. In some cases, Het is an aromatic 5-7 membered heterocycle having 1-3 ring heteroatoms. In some cases, Het is a non-aromatic 4-7 membered heterocycle having 1-3 ring heteroatoms. In some cases, Het is unsubstituted. In some cases, Het is substituted. Het can be substituted with C₁₋₆alkyl. Het can be substituted with C₁₋₆alkoxy. Het can be substituted with C(O)R^{N} or SO₂R^{N}. In some cases, Het is a non-aromatic 4-7 membered heterocycle and is substituted with oxo.

In various cases, R³ is C₁₋₆alkylene-X. In some cases, R³ is is C₂₋₆alkenylene-X or C₀₋₂alkylene-C₃₋₆ carbocycle-C₀₋₂alkylene-X. In some cases, the R³ alkylene is substituted with OR^{N} (e.g., OH or OMe).

In various cases, X is H, OC₁₋₃alkyl, CN, CO₂R^{N}, or CON(R^{N})₂. In some cases, X is C≡CR^{N}. In some cases, X is Ar. In some cases, R³ is Ar. In some cases, Ar is 3-10 membered non-aromatic monocyclic or polycyclic ring having 0-4 ring heteroatoms selected from N, O, and S. In some cases, Ar is a 5-10 membered aromatic monocyclic or polycyclic ring having 0-4 ring heteroatoms selected from N, O, and S. In some case, Ar is phenyl. In some cases, Ar is a 5-10 membered aromatic monocyclic or polycyclic ring having 1-4 ring heteroatoms selected from N, O, and S. In some cases, Ar is a 6-10 membered aromatic monocyclic or polycyclic ring having 2-4 ring heteroatoms selected from N, O, and S. In some cases, Ar is phenyl, tetrahydropyran, dihydropyran, tetrahydrofuran, C₃₋₆cycloalkyl, tetrazole, triazole, oxazole, tetrahydroquinoline, N-methyl-tetrahydroisoquinoline, tetrahydrothiopyranyl-dioxide, pyridinone, piperidinone, or oxetanyl. Ar can be substituted or unsubstituted. In some cases, Ar is substituted, optionally with at least one substituent meta to point of attachment, e.g., when Ar is phenyl:

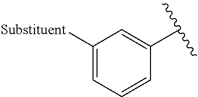

(where phenyl can be further substituted with a second substituent). In some cases, Ar is substituted with C₁₋₃alkyl, C₀₋₂alklene-CN, or CON(R^{N})₂. In some cases, Ar is substituted with 1 or 2 halo (e.g., fluoro). In some cases, R³ is

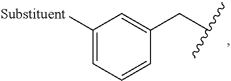

and in some specific cases the substituent is halo (e.g., fluoro).

In various cases, o is 0. In some cases, o is 1, and in some specific cases, R^{z} is meta to the ring nitrogen, i.e.,

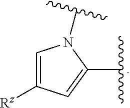

In various cases, the compound of Formula (III) is a structure as shown in Table C, or a pharmaceutically acceptable salt thereof:

TABLE C

| | |
|---|---|
| C1 | 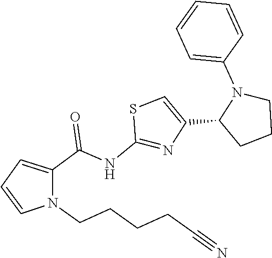 [SMILES: N#CCCCCn1cccc1C(=O)Nc1nc([C@H]2CCCN2c2ccccc2)cs1] |
| C2 | 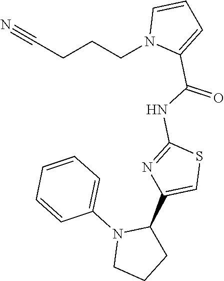 [SMILES: N#CCCCn1cccc1C(=O)Nc1nc([C@H]2CCCN2c2ccccc2)cs1] |
| C3 | 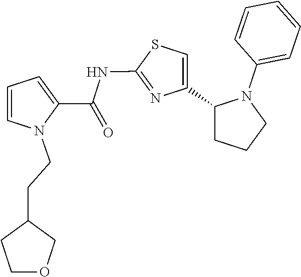 [SMILES: O=C(Nc1nc([C@H]2CCCN2c2ccccc2)cs1)c1cccn1CCC1CCOC1] |
| C4 | 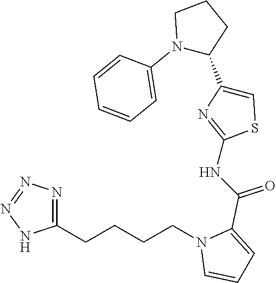 [SMILES: O=C(Nc1nc([C@H]2CCCN2c2ccccc2)cs1)c1cccn1CCCCc1nnn[nH]1] |
| C5 | 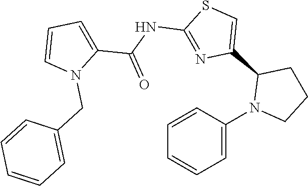 [SMILES: O=C(Nc1nc([C@H]2CCCN2c2ccccc2)cs1)c1cccn1Cc1ccccc1] |
| C6 | 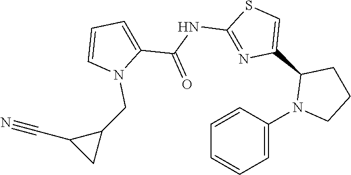 [SMILES: N#CC1CC1Cn1cccc1C(=O)Nc1nc([C@H]2CCCN2c2ccccc2)cs1] |
| C7 | 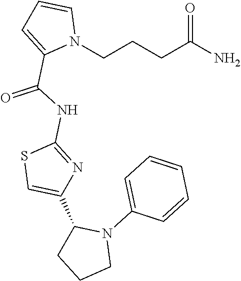 [SMILES: NC(=O)CCCn1cccc1C(=O)Nc1nc([C@H]2CCCN2c2ccccc2)cs1] |
| C8 | 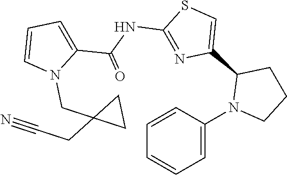 [SMILES: N#CCC1(Cn2cccc2C(=O)Nc2nc([C@H]3CCCN3c3ccccc3)cs2)CC1] |
| C9 | 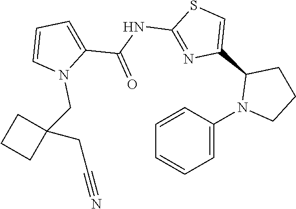 [SMILES: N#CCC1(Cn2cccc2C(=O)Nc2nc([C@H]3CCCN3c3ccccc3)cs2)CCC1] |
| C10 | 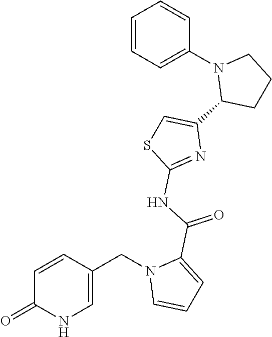 [SMILES: O=C(Nc1nc([C@H]2CCCN2c2ccccc2)cs1)c1cccn1Cc1ccc(=O)[nH]c1] |
| C11 | 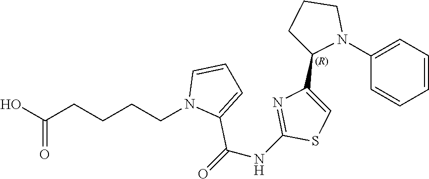 |
| C12 | 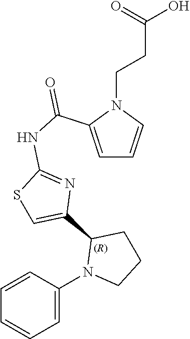 |
| C13 | 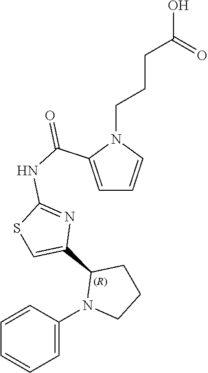 |
| C14 | 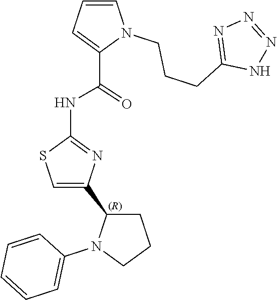 |
| C15 | 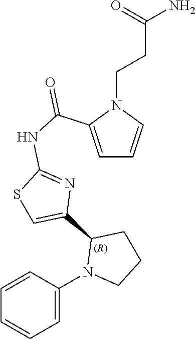 |
| C16 | 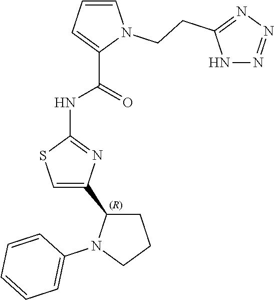 |
| C17 | 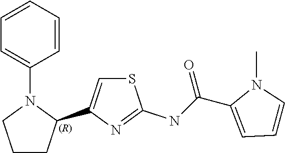 |
| C18 | 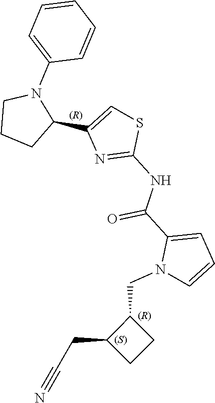 |
| C19 | 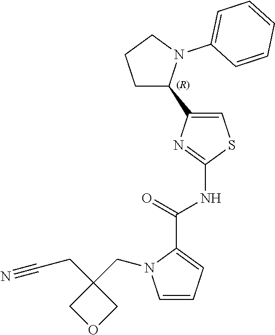 |
| C20 | 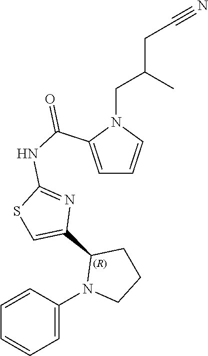 |
| C21 | 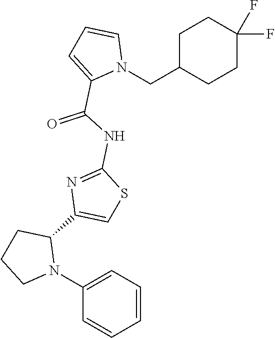 [SMILES: O=C(Nc1nc([C@H]2CCCN2c2ccccc2)cs1)c1cccn1CC1CCC(F)(F)CC1] |
| C22 | 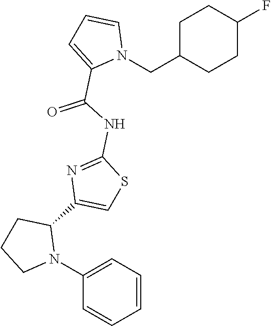 [SMILES: O=C(Nc1nc([C@H]2CCCN2c2ccccc2)cs1)c1cccn1CC1CCC(F)CC1] |
| C23 | 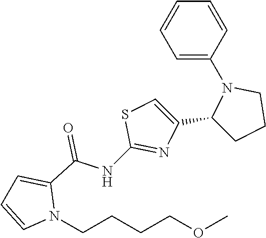 [SMILES: COCCCCn1cccc1C(=O)Nc1nc([C@H]2CCCN2c2ccccc2)cs1] |
| C24 | 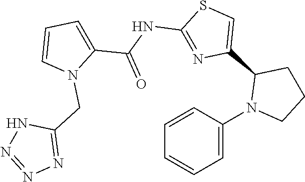 [SMILES: O=C(Nc1nc([C@H]2CCCN2c2ccccc2)cs1)c1cccn1Cc1nnn[nH]1] |
| C25 | 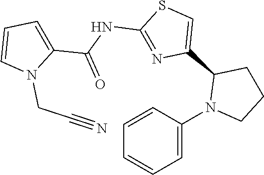 [SMILES: N#CCn1cccc1C(=O)Nc1nc([C@H]2CCCN2c2ccccc2)cs1] |
| C26 | 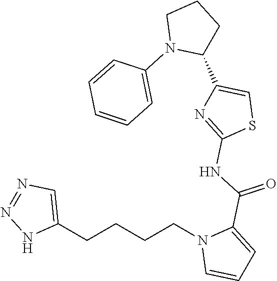 [SMILES: O=C(Nc1nc([C@H]2CCCN2c2ccccc2)cs1)c1cccn1CCCCc1cnn[nH]1] |
| C27 | 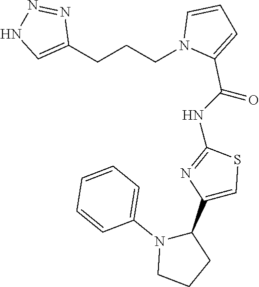 [SMILES: O=C(Nc1nc([C@H]2CCCN2c2ccccc2)cs1)c1cccn1CCCc1c[nH]nn1] |
| C28 | 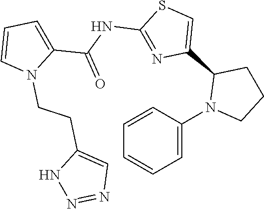 [SMILES: O=C(Nc1nc([C@H]2CCCN2c2ccccc2)cs1)c1cccn1CCc1cnn[nH]1] |
| C29 | 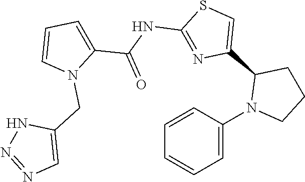 [SMILES: O=C(Nc1nc([C@H]2CCCN2c2ccccc2)cs1)c1cccn1Cc1cnn[nH]1] |
| C30 | 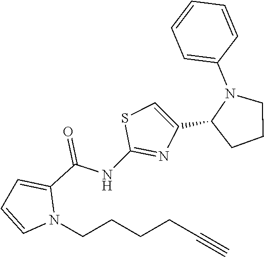 [SMILES: C#CCCCCn1cccc1C(=O)Nc1nc([C@H]2CCCN2c2ccccc2)cs1] |
| C31 | 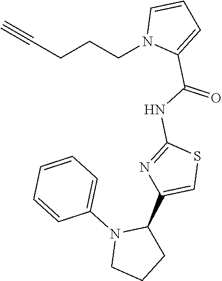 [SMILES: C#CCCCn1cccc1C(=O)Nc1nc([C@H]2CCCN2c2ccccc2)cs1] |
| C32 | 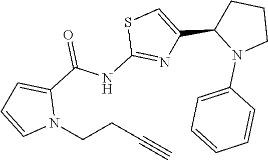 [SMILES: C#CCCn1cccc1C(=O)Nc1nc([C@H]2CCCN2c2ccccc2)cs1] |
| C33 | 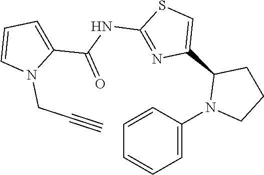 [SMILES: C#CCn1cccc1C(=O)Nc1nc([C@H]2CCCN2c2ccccc2)cs1] |
| C34 | 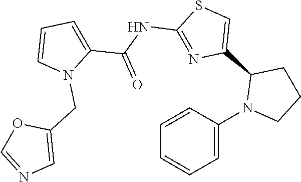 [SMILES: O=C(Nc1nc([C@H]2CCCN2c2ccccc2)cs1)c1cccn1Cc1cnco1] |
| C35 | 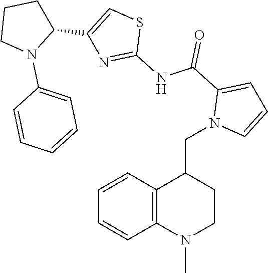 [SMILES: CN1CCC(Cn2cccc2C(=O)Nc2nc([C@H]3CCCN3c3ccccc3)cs2)c2ccccc21] |
| C36 | 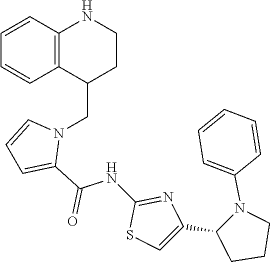 [SMILES: O=C(Nc1nc([C@H]2CCCN2c2ccccc2)cs1)c1cccn1CC1CCNc2ccccc21] |
| C37 | 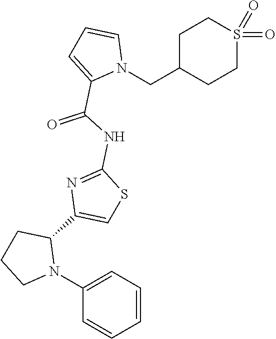 [SMILES: O=C(Nc1nc([C@H]2CCCN2c2ccccc2)cs1)c1cccn1CC1CCS(=O)(=O)CC1] |
| C38 | 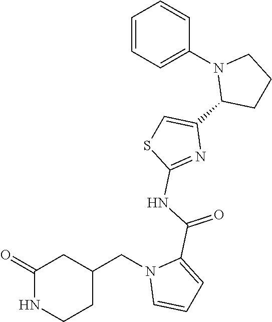 [SMILES: O=C1CC(Cn2cccc2C(=O)Nc2nc([C@H]3CCCN3c3ccccc3)cs2)CCN1] |
| C39 | 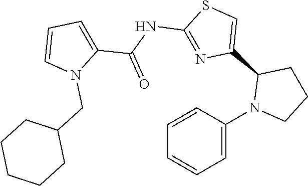 [SMILES: O=C(Nc1nc([C@H]2CCCN2c2ccccc2)cs1)c1cccn1CC1CCCCC1] |
| C40 | 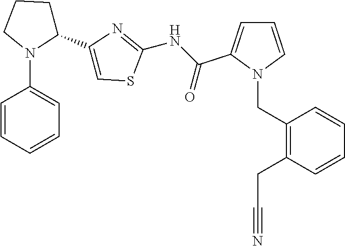 [SMILES: N#CCc1ccccc1Cn1cccc1C(=O)Nc1nc([C@H]2CCCN2c2ccccc2)cs1] |
| C41 | 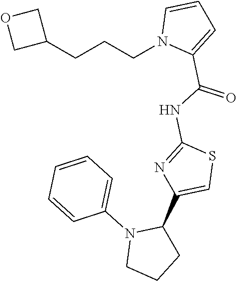 [SMILES: O=C(Nc1nc([C@H]2CCCN2c2ccccc2)cs1)c1cccn1CCCC1COC1] |
| C42 | 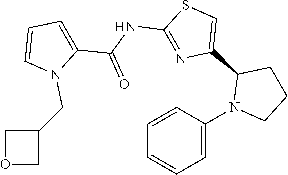 [SMILES: O=C(Nc1nc([C@H]2CCCN2c2ccccc2)cs1)c1cccn1CC1COC1] |
| C43 | 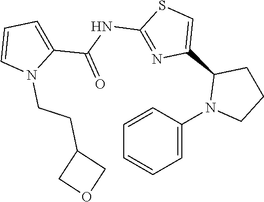 [SMILES: O=C(Nc1nc([C@H]2CCCN2c2ccccc2)cs1)c1cccn1CCC1COC1] |
| C44 | 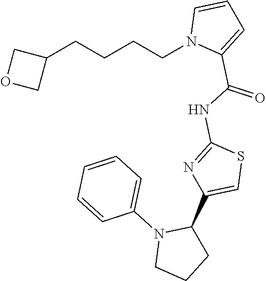 [SMILES: O=C(Nc1nc([C@H]2CCCN2c2ccccc2)cs1)c1cccn1CCCCC1COC1] |
| C45 | 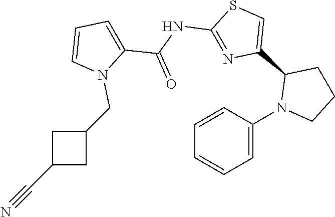 [SMILES: N#CC1CC(Cn2cccc2C(=O)Nc2nc([C@H]3CCCN3c3ccccc3)cs2)C1] |
| C46 | 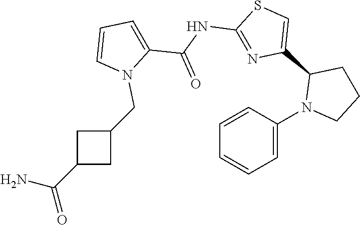 [SMILES: NC(=O)C1CC(Cn2cccc2C(=O)Nc2nc([C@H]3CCCN3c3ccccc3)cs2)C1] |
| C47 | 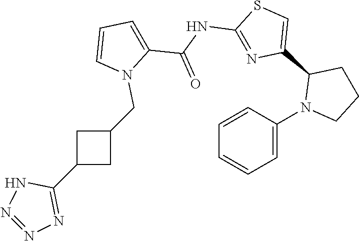 [SMILES: O=C(Nc1nc([C@H]2CCCN2c2ccccc2)cs1)c1cccn1CC1CC(c2nnn[nH]2)C1] |
| C48 | 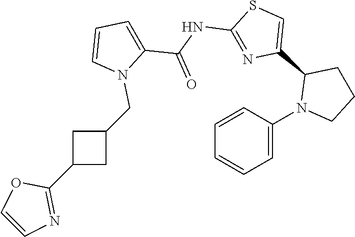 [SMILES: O=C(Nc1nc([C@H]2CCCN2c2ccccc2)cs1)c1cccn1CC1CC(c2ncco2)C1] |
| C49 | 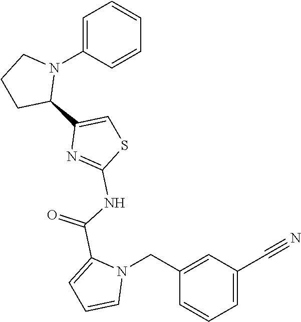 [SMILES: N#Cc1cccc(Cn2cccc2C(=O)Nc2nc([C@H]3CCCN3c3ccccc3)cs2)c1] |
| C50 | 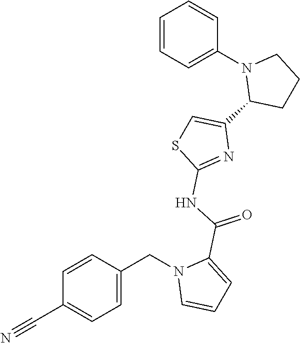 [SMILES: N#Cc1ccc(Cn2cccc2C(=O)Nc2nc([C@H]3CCCN3c3ccccc3)cs2)cc1] |
| C51 | 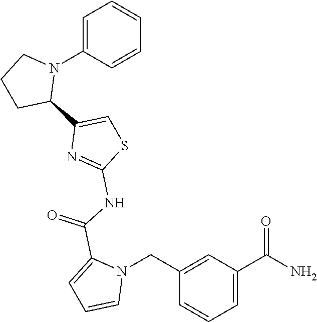 [SMILES: NC(=O)c1cccc(Cn2cccc2C(=O)Nc2nc([C@H]3CCCN3c3ccccc3)cs2)c1] |
| C52 | 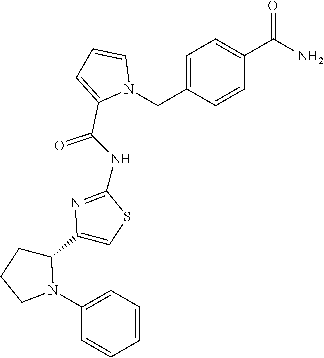 [SMILES: NC(=O)c1ccc(Cn2cccc2C(=O)Nc2nc([C@H]3CCCN3c3ccccc3)cs2)cc1] |
| C53 | 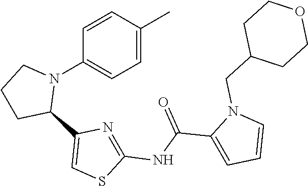 [SMILES: Cc1ccc(N2CCC[C@@H]2c2csc(NC(=O)c3cccn3CC3CCOCC3)n2)cc1] |
| C54 | 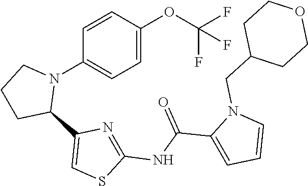 [SMILES: O=C(Nc1nc([C@H]2CCCN2c2ccc(OC(F)(F)F)cc2)cs1)c1cccn1CC1CCOCC1] |
| C55 | 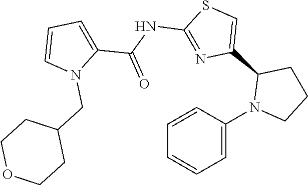 [SMILES: O=C(Nc1nc([C@H]2CCCN2c2ccccc2)cs1)c1cccn1CC1CCOCC1] |
| C56 | 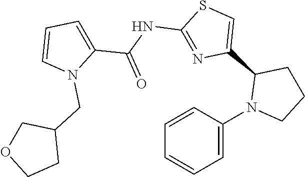 [SMILES: O=C(Nc1nc([C@H]2CCCN2c2ccccc2)cs1)c1cccn1CC1CCOC1] |
| C57 | 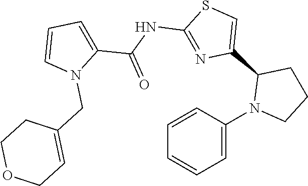 [SMILES: O=C(Nc1nc([C@H]2CCCN2c2ccccc2)cs1)c1cccn1CC1=CCOCC1] |
| C58 | 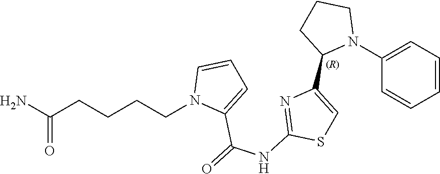 |
| C59 | 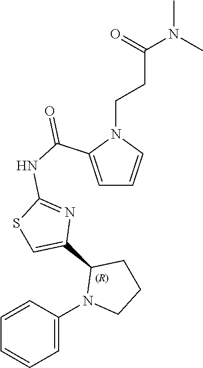 |
| C60 | 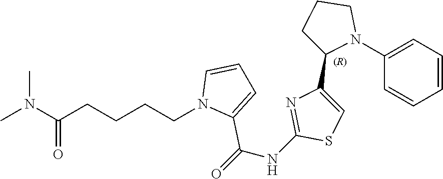 |
| C61 | 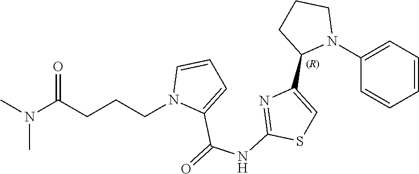 |
| C62 | 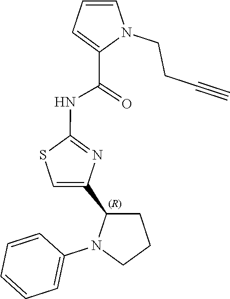 |
| C63 | 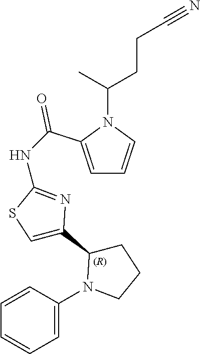 |
| C64 | 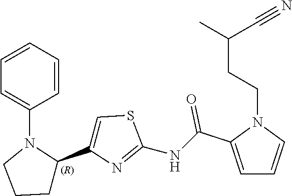 |
| C65 | 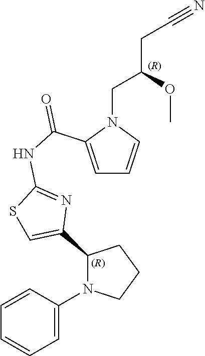 |
| C66 | 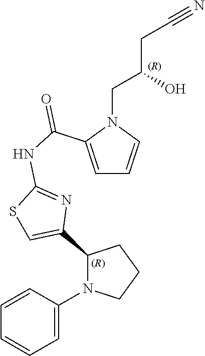 |
| C67 | 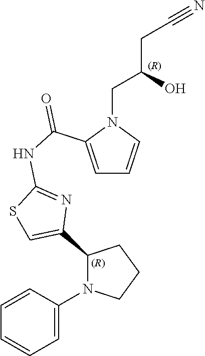 |
| C68 | 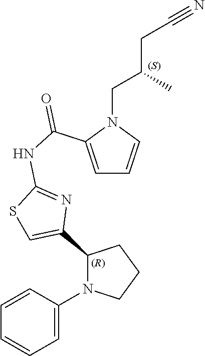 |
| C69 | 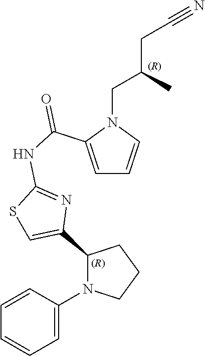 |
| C70 | 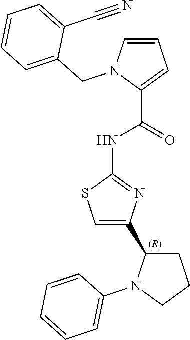 |
| C71 | 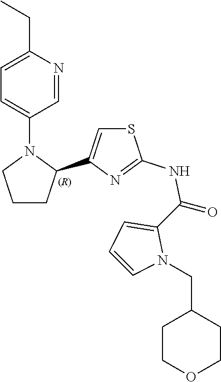 |
| C72 | 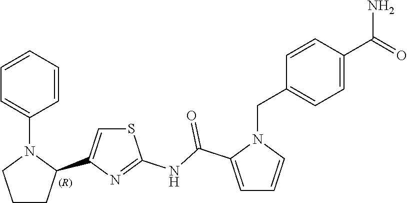 |
| C73 | 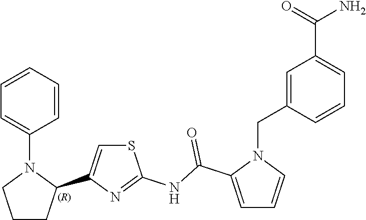 |
| C74 | 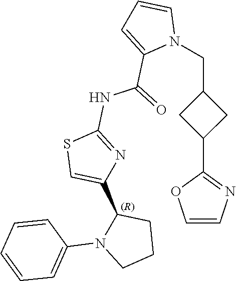 |
| C75 | 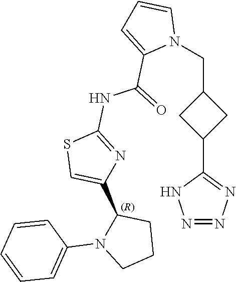 |
| C76 | 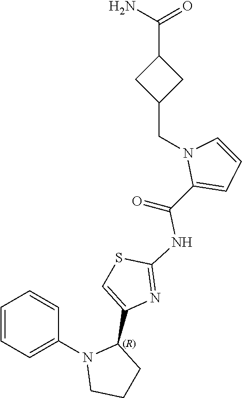 |
| C77 | 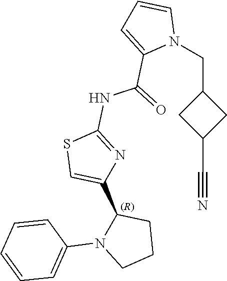 |
| C78 | 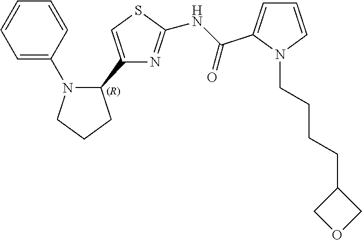 |
| C79 | 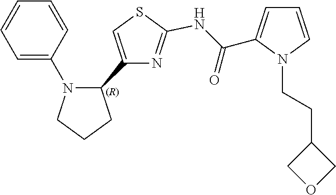 |
| C80 | 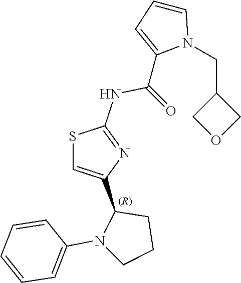 |
| C81 | 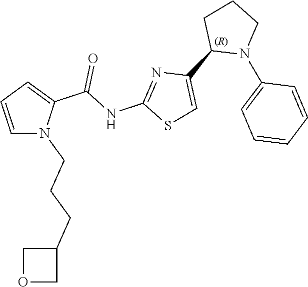 |
| C82 | 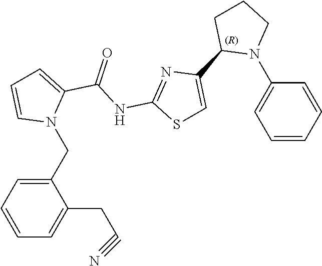 |
| C83 | 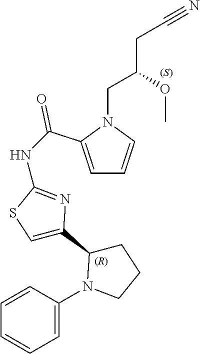 |
| C84 | 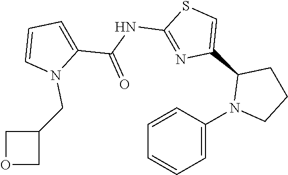 [SMILES: O=C(Nc1nc([C@H]2CCCN2c2ccccc2)cs1)c1cccn1CC1COC1] |
| C85 | 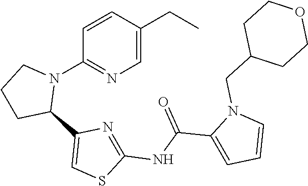 [SMILES: CCc1ccc(N2CCC[C@@H]2c2csc(NC(=O)c3cccn3CC3CCOCC3)n2)nc1] |
| C86 | 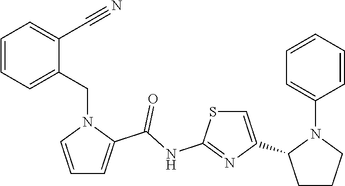 [SMILES: N#Cc1ccccc1Cn1cccc1C(=O)Nc1nc([C@H]2CCCN2c2ccccc2)cs1] |
| C87 | 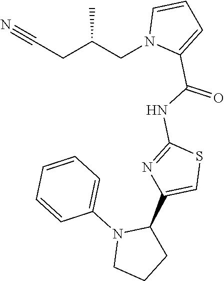 [SMILES: C[C@@H](CC#N)Cn1cccc1C(=O)Nc1nc([C@H]2CCCN2c2ccccc2)cs1] |
| C88 | 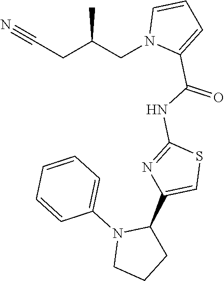 [SMILES: C[C@H](CC#N)Cn1cccc1C(=O)Nc1nc([C@H]2CCCN2c2ccccc2)cs1] |
| C89 | 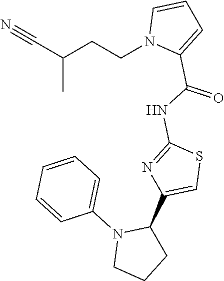 [SMILES: CC(C#N)CCn1cccc1C(=O)Nc1nc([C@H]2CCCN2c2ccccc2)cs1] |
| C90 | 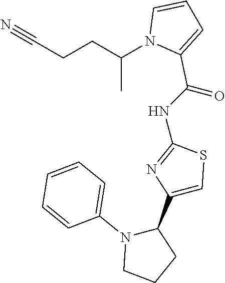 [SMILES: CC(CCC#N)n1cccc1C(=O)Nc1nc([C@H]2CCCN2c2ccccc2)cs1] |
| C91 | 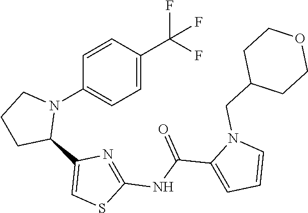 [SMILES: O=C(Nc1nc([C@H]2CCCN2c2ccc(C(F)(F)F)cc2)cs1)c1cccn1CC1CCOCC1] |
| C92 | 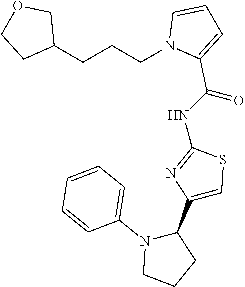 [SMILES: O=C(Nc1nc([C@H]2CCCN2c2ccccc2)cs1)c1cccn1CCCC1CCOC1] |
| C93 | 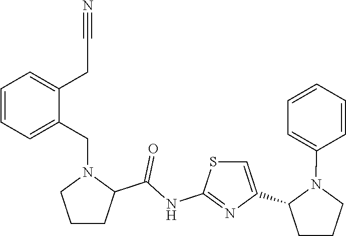 [SMILES: N#CCc1ccccc1CN1CCCC1C(=O)Nc1nc([C@H]2CCCN2c2ccccc2)cs1] |
| C94 | 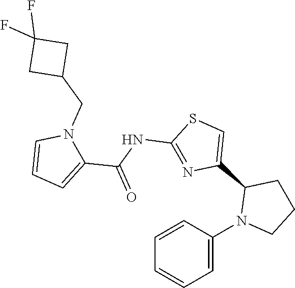 [SMILES: O=C(Nc1nc([C@H]2CCCN2c2ccccc2)cs1)c1cccn1CC1CC(F)(F)C1] |
| C95 | 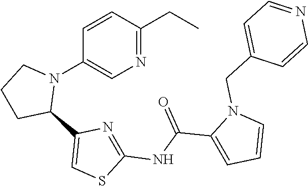 |
| C96 | 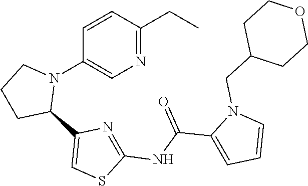 |
| C97 | 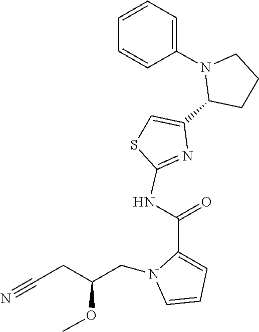 [SMILES: CO[C@@H](CC#N)Cn1cccc1C(=O)Nc1nc([C@H]2CCCN2c2ccccc2)cs1] |
| C99 | 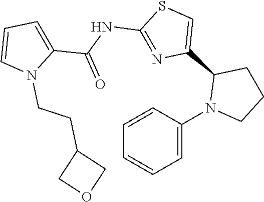 [SMILES: O=C(Nc1nc([C@H]2CCCN2c2ccccc2)cs1)c1cccn1CCC1COC1] |
| C100 | 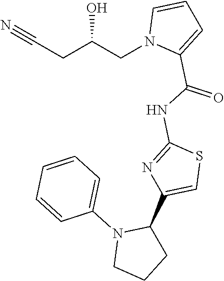 [SMILES: N#CC[C@H](O)Cn1cccc1C(=O)Nc1nc([C@H]2CCCN2c2ccccc2)cs1] |
| C102 | 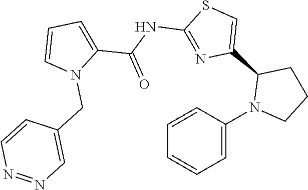 [SMILES: O=C(Nc1nc([C@H]2CCCN2c2ccccc2)cs1)c1cccn1Cc1ccnnc1] |
| C103 | 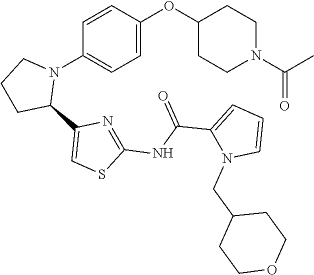 [SMILES: CC(=O)N1CCC(Oc2ccc(N3CCC[C@@H]3c3csc(NC(=O)c4cccn4CC4CCOCC4)n3)cc2)CC1] |
| C104 | 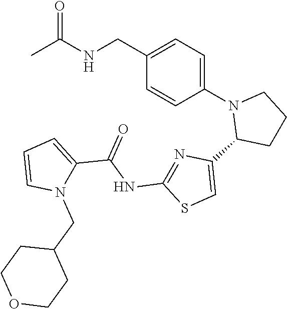 [SMILES: CC(=O)NCc1ccc(N2CCC[C@@H]2c2csc(NC(=O)c3cccn3CC3CCOCC3)n2)cc1] |
| C105 | 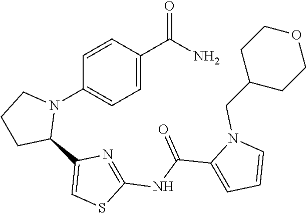 [SMILES: NC(=O)c1ccc(N2CCC[C@@H]2c2csc(NC(=O)c3cccn3CC3CCOCC3)n2)cc1] |
| C106 | 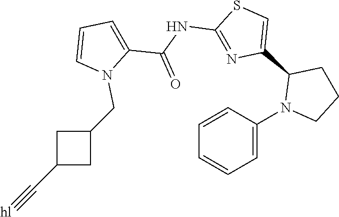 |
| C107 | 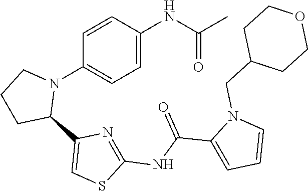 [SMILES: CC(=O)Nc1ccc(N2CCC[C@@H]2c2csc(NC(=O)c3cccn3CC3CCOCC3)n2)cc1] |
| C108 | 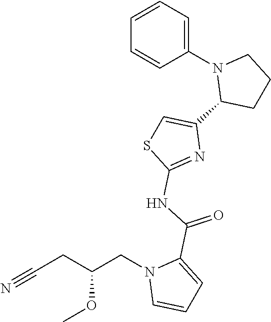 [SMILES: CO[C@H](CC#N)Cn1cccc1C(=O)Nc1nc([C@H]2CCCN2c2ccccc2)cs1] |
| C110 | 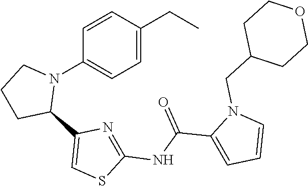 [SMILES: CCc1ccc(N2CCC[C@@H]2c2csc(NC(=O)c3cccn3CC3CCOCC3)n2)cc1] |
| C111 | 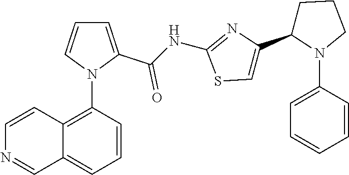 [SMILES: O=C(Nc1nc([C@H]2CCCN2c2ccccc2)cs1)c1cccn1-c1cccc2cnccc12] |
| C112 | 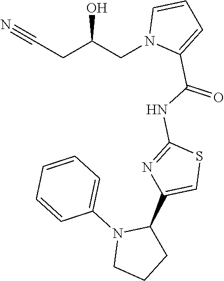 [SMILES: N#CC[C@@H](O)Cn1cccc1C(=O)Nc1nc([C@H]2CCCN2c2ccccc2)cs1] |
| C113 | 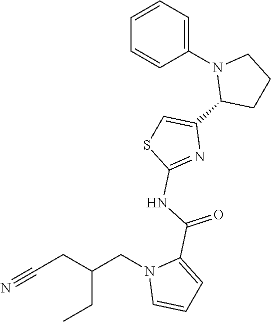 [SMILES: CCC(CC#N)Cn1cccc1C(=O)Nc1nc([C@H]2CCCN2c2ccccc2)cs1] |
| C114 | 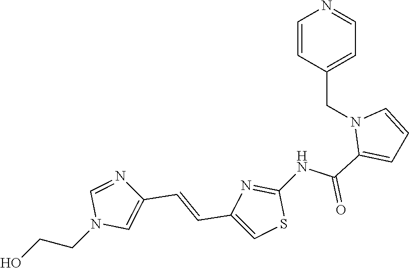 [SMILES: O=C(Nc1nc(/C=C/c2cn(CCO)cn2)cs1)c1cccn1Cc1ccncc1] |
| C115 | 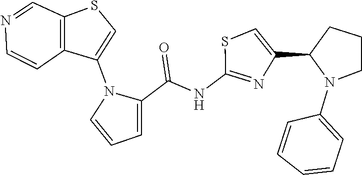 [SMILES: O=C(Nc1nc([C@H]2CCCN2c2ccccc2)cs1)c1cccn1-c1csc2cnccc12] |
| C116 | 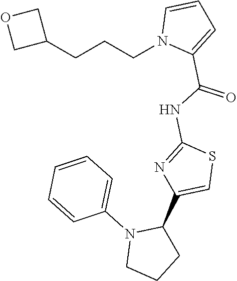 [SMILES: O=C(Nc1nc([C@H]2CCCN2c2ccccc2)cs1)c1cccn1CCCC1COC1] |
| C117 | 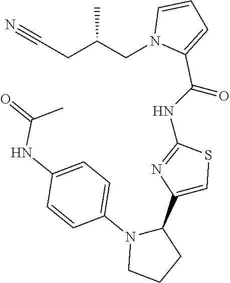 [SMILES: CC(=O)Nc1ccc(N2CCC[C@@H]2c2csc(NC(=O)c3cccn3C[C@@H](C)CC#N)n2)cc1] |
| C118 | 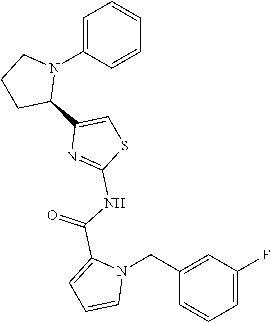 [SMILES: O=C(Nc1nc([C@H]2CCCN2c2ccccc2)cs1)c1cccn1Cc1cccc(F)c1] |
| C119 | 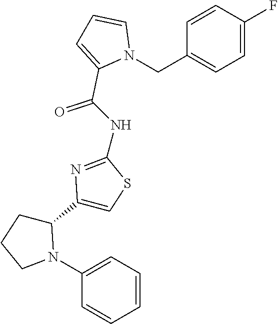 [SMILES: O=C(Nc1nc([C@H]2CCCN2c2ccccc2)cs1)c1cccn1Cc1ccc(F)cc1] |
| C120 | 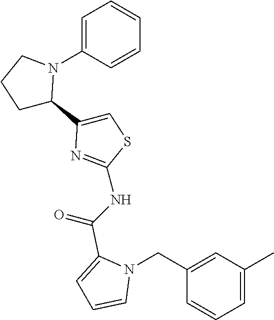 [SMILES: Cc1cccc(Cn2cccc2C(=O)Nc2nc([C@H]3CCCN3c3ccccc3)cs2)c1] |
| C121 | 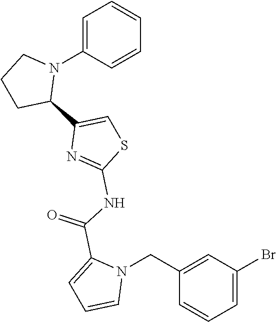 [SMILES: O=C(Nc1nc([C@H]2CCCN2c2ccccc2)cs1)c1cccn1Cc1cccc(Br)c1] |
| C122 | 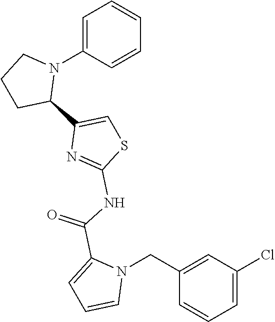 [SMILES: O=C(Nc1nc([C@H]2CCCN2c2ccccc2)cs1)c1cccn1Cc1cccc(Cl)c1] |
| C123 | 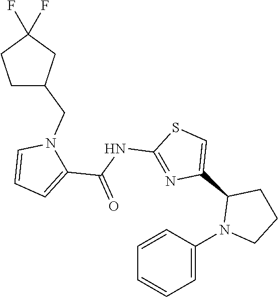 [SMILES: O=C(Nc1nc([C@H]2CCCN2c2ccccc2)cs1)c1cccn1CC1CCC(F)(F)C1] |
| C124 | 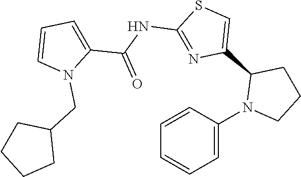 [SMILES: O=C(Nc1nc([C@H]2CCCN2c2ccccc2)cs1)c1cccn1CC1CCCC1] |
| C125 | 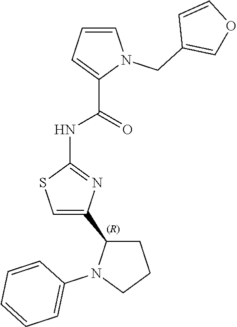 |
| C126 | 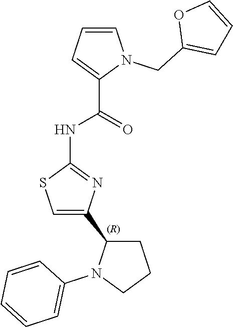 |
| C127 | 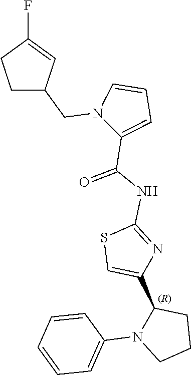 |
| C128 | 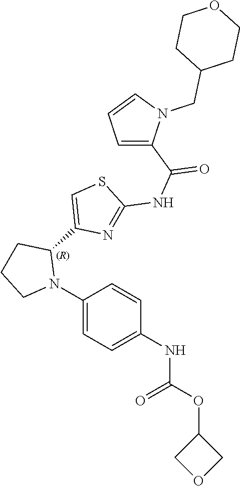 |
| C129 | 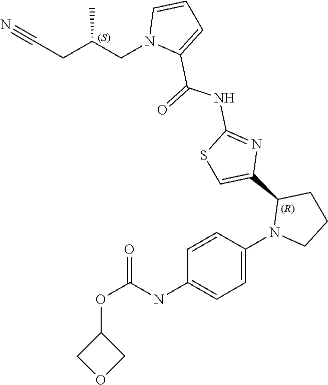 |
| C131 | 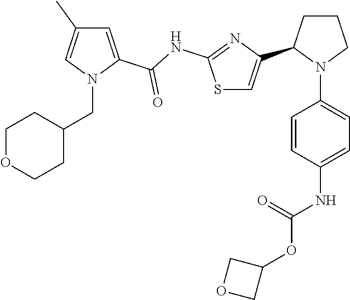 [SMILES: Cc1cc(C(=O)Nc2nc([C@H]3CCCN3c3ccc(NC(=O)OC4COC4)cc3)cs2)n(CC2CCOCC2)c1] |
| C132 | 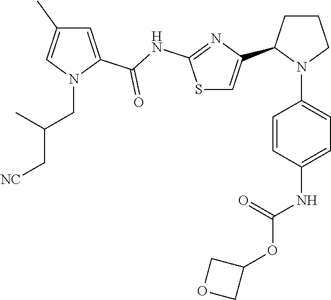 [SMILES: Cc1cc(C(=O)Nc2nc([C@H]3CCCN3c3ccc(NC(=O)OC4COC4)cc3)cs2)n(CC(C)CC#N)c1] |

### Compounds of Formula (IV)

Also provided herein are compounds of Formula (IV), or pharmaceutically acceptable salts thereof, having a structure of:

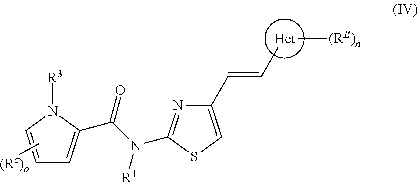

R¹ is H, C₁₋₃alkyl, or SO₂C₁₋₆alkyl;
Het is 3-10 membered aromatic or non-aromatic heterocycle having 1-4 ring heteroatoms selected from N, O, and S;
n is 0, 1, or 2; and
each R^{E}, when present, is independently halo, C₁₋₆alkyl, phenyl, C(O)N(R^{N})₂, CN, C₀₋₆alkylene-OR^{N}, C₀₋₆alkylene-N(R^{N})₂, C₁₋₆haloalkyl, C₁₋₆haloalkoxy, C₃₋₆cycloalkyl, or CO₂R^{N};
   wherein when R^{E} is phenyl, it is optionally substituted with 1-2 groups independently selected from halo, C₁₋₆alkyl, CN, C₁₋₆haloalkyl, C₁₋₆haloalkoxy, CO₂R^{N}, CON(R^{N})₂, N(R^{N})COR^{N}, and OR^{N};
R³ is C₁₋₆alkylene-X, C₂₋₆alkenylene-X, Ar, or C₀₋₂alkylene-C₃₋₆carbocycle-C₀₋₂alkylene-X;
X is H, OC₁₋₃alkyl, C≡CR^{N}; CN, CO₂R^{N}; CON(R^{N})₂, or Ar,
Ar is a 3-10 membered aromatic or non-aromatic ring having 0-4 ring heteroatoms selected from N, O, and S, with the proviso that when Ar is a 6-membered aromatic ring, it has 0 or 2-4 ring heteroatoms;
Ar is optionally substituted with C₁₋₃alkyl, C₀₋₂alklene-CN, CON(R^{N})₂, tetrazole, oxazole, or 1-2 halo;
o is 0 or 1;
R^{z}, when present, is CN, halo, C(O)N(R^{N})₂, C₁₋₆alkyl, C₁₋₆alkoxy, C₁₋₆hydroxyalkyl, or C₁₋₆haloalkyl; and
each R^{N} is independently H, C₁₋₆alkyl, C₁₋₆hydroxyalkyl, or C₁₋₆haloalkyl.

In various cases, R¹ is H.

In various case, Het is a 3-10 membered non-aromatic heterocycle having 1-4 ring heteroatoms selected from N, O, and S. In some cases, Het is tetrahydropyran. In some cases, Het is a 5-10 membered aromatic heterocycle having 1-4 ring heteroatoms selected from N, O, and S. in some cases, Het is oxazole. In some cases, Het is imidazole. In some cases, Het is diazinyl-pyrimidinyl, pyrazinyl, or pyradazinyl. In some cases, Het is isoxazole, morpholine, tetrahydroquinoline, oxazolindinone, piperidinone, or dihydrooxazole.

Het can be unsubstituted (i.e., n is 0). Het can be substituted with R^{E} (i.e., n is 1 or 2). In some cases, at least one R^{E} is halo (e.g., fluoro). In some cases, wherein at least one R^{E} is C₁₋₆alkyl or C(O)N(R^{N})₂. In some cases, at least one R^{E} is C₀₋₆alkylene-OR^{N} or C₀₋₆alkylene-N(R^{N})₂. In some cases, at least one R^{E} is phenyl. The phenyl can be substituted or unsubstituted. In some cases, the phenyl is substituted with 1 substitutent selected from halo, C₁₋₆haloalkyl, C₁₋₆haloalkoxy, CON(R^{N})₂, N(R^{N})COR^{N} and OR^{N}.

In some cases, R³ is C₁₋₆alkylene-X. In some cases, R³ C₂₋₆alkenylene-X or C₀₋₂alkylene-C₃₋₆ carbocycle-C₀₋₂alkylene-X. In some cases, X is H, OC₁₋₃alkyl, CN, CO₂R^{N}, or CON(R^{N})₂. In some cases, X is C≡CR^{N}. In some cases, X is Ar. In some cases, Ar is a 3-10 membered non-aromatic monocyclic or polycyclic ring having 0-4 ring heteroatoms selected from N, O, and S. In some cases, Ar is a 5-10 membered aromatic monocyclic or polycyclic ring having 0-4 ring heteroatoms selected from N, O, and S. In some cases, Ar is phenyl. In some cases, Ar is a 5-10 membered aromatic monocyclic or polycyclic ring having 1-4 ring heteroatoms selected from N, O, and S. In some cases, Ar is a 5 or 7-10 membered aromatic monocyclic or polycyclic ring having 1-4 ring heteroatoms selected from N, O, and S. In some cases, Ar is a 6-10 membered aromatic monocyclic or polycyclic ring having 2-4 ring heteroatoms selected from N, O, and S. In some cases, Ar is phenyl, tetrahydropyran, dihydropyran, tetrahydrofuran, C₃₋₆cycloalkyl, tetrazole, triazole, oxazole, tetrahydroquinoline, N-methyl-tetrahydroisoquinoline, tetrahydrothiopyranyl-dioxide, pyridinone, piperidinone, or oxetanyl. Ar can be substituted or unsubstituted. In some cases, Ar is substituted optionally meta to point of attachment, e.g., when Ar is phenyl:

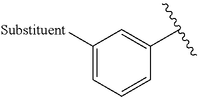

(where phenyl can be further substituted with a second substituent). In some cases, Ar is substituted with C₁₋₃alkyl, C₀₋₂alklene-CN, or CON(R^{N})₂. In some cases, Ar is substituted with 1 or 2 halo (e.g., fluoro). In some cases, R³ is

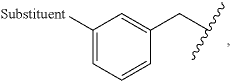

and in some specific cases the substituent is halo (e.g., fluoro).

In various cases, o is 0. In some cases, o is 1, and in some specific cases, R^{z} is meta to the ring nitrogen, i.e.,

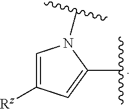

In various cases, the compound of Formula (IV) is a structure as shown in Table D, or a pharmaceutically acceptable salt thereof:

TABLE D

| ID | |
|---|---|
| D1 | 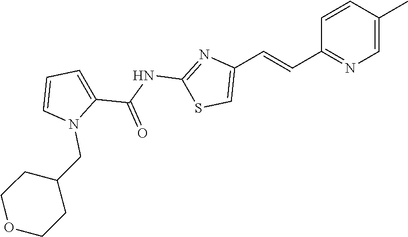 [SMILES: Cc1ccc(/C=C/c2csc(NC(=O)c3cccn3CC3CCOCC3)n2)nc1] |
| D2 | 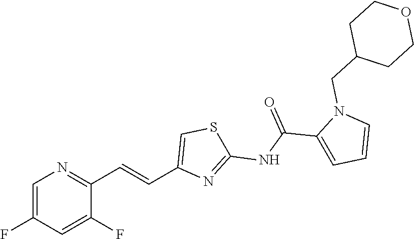 [SMILES: O=C(Nc1nc(/C=C/c2ncc(F)cc2F)cs1)c1cccn1CC1CCOCC1] |
| D3 | 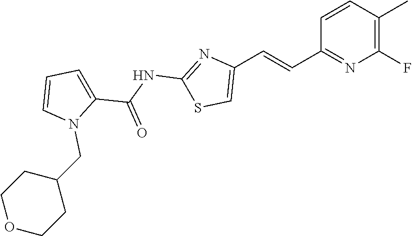 [SMILES: Cc1ccc(/C=C/c2csc(NC(=O)c3cccn3CC3CCOCC3)n2)nc1F] |
| D4 | 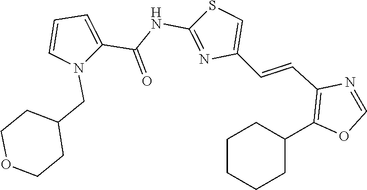 [SMILES: O=C(Nc1nc(/C=C/c2ncoc2C2CCCCC2)cs1)c1cccn1CC1CCOCC1] |
| D5 | 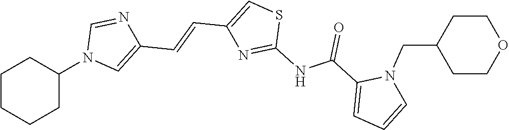 [SMILES: O=C(Nc1nc(/C=C/c2cn(C3CCCCC3)cn2)cs1)c1cccn1CC1CCOCC1] |
| D6 | 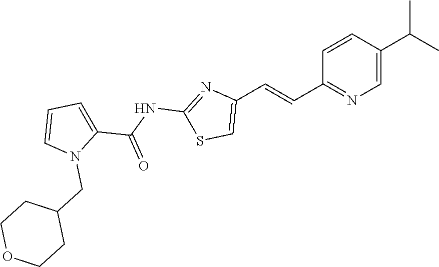 [SMILES: CC(C)c1ccc(/C=C/c2csc(NC(=O)c3cccn3CC3CCOCC3)n2)nc1] |
| D7 | 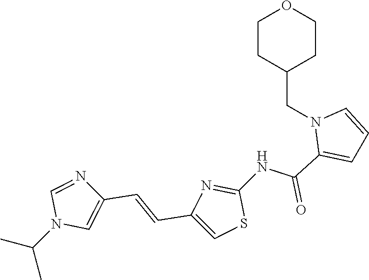 [SMILES: CC(C)n1cnc(/C=C/c2csc(NC(=O)c3cccn3CC3CCOCC3)n2)c1] |
| D8 | 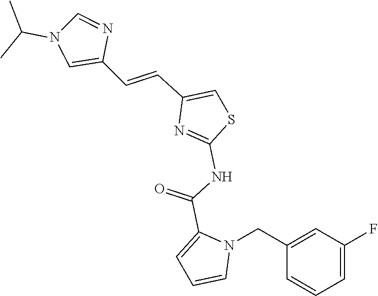 [SMILES: CC(C)n1cnc(/C=C/c2csc(NC(=O)c3cccn3Cc3cccc(F)c3)n2)c1] |

Further provided herein are compounds as shown in Table E, or pharmaceutically acceptable salts thereof:

TABLE E

| ID | |
|---|---|
| E1 | 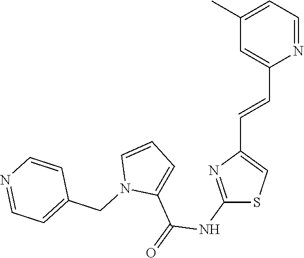 [SMILES: Cc1ccnc(/C=C/c2csc(NC(=O)c3cccn3Cc3ccncc3)n2)c1] |
| E2 | 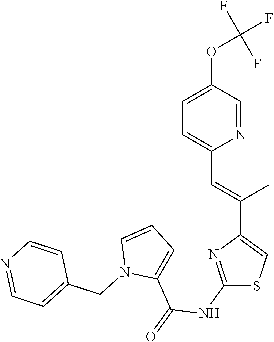 [SMILES: C/C(=C\c1ccc(OC(F)(F)F)cn1)c1csc(NC(=O)c2cccn2Cc2ccncc2)n1] |
| E3 | 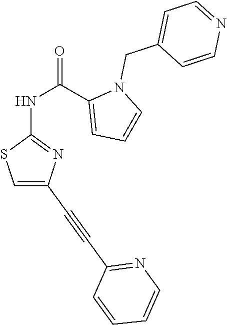 [SMILES: O=C(Nc1nc(C#Cc2ccccn2)cs1)c1cccn1Cc1ccncc1] |
| E4 | 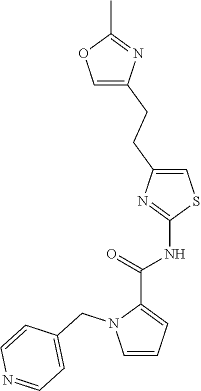 [SMILES: Cc1nc(CCc2csc(NC(=O)c3cccn3Cc3ccncc3)n2)co1] |
| E5 | 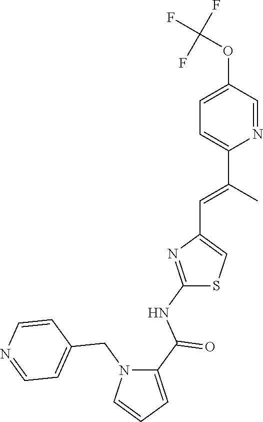 [SMILES: C/C(=C\c1csc(NC(=O)c2cccn2Cc2ccncc2)n1)c1ccc(OC(F)(F)F)cn1] |
| E6 | 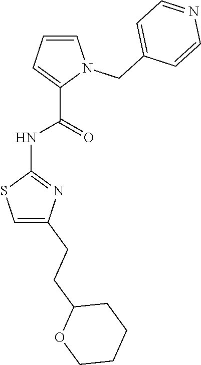 [SMILES: O=C(Nc1nc(CCC2CCCCO2)cs1)c1cccn1Cc1ccncc1] |
| E7 | 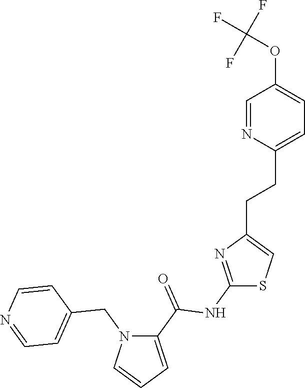 [SMILES: O=C(Nc1nc(CCc2ccc(OC(F)(F)F)cn2)cs1)c1cccn1Cc1ccncc1] |
| E8 | 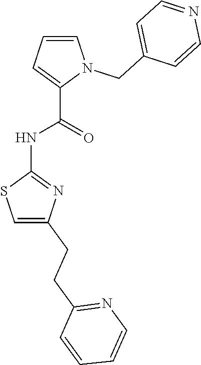 [SMILES: O=C(Nc1nc(CCc2ccccn2)cs1)c1cccn1Cc1ccncc1] |
| E9 | 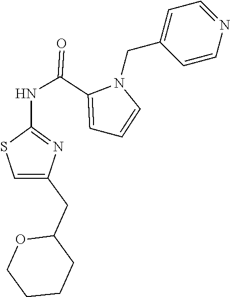 [SMILES: O=C(Nc1nc(CC2CCCCO2)cs1)c1cccn1Cc1ccncc1] |
| E10 | 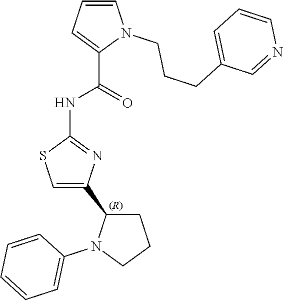 |
| E11 | 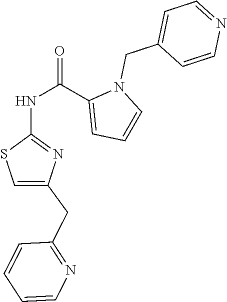 [SMILES: O=C(Nc1nc(Cc2ccccn2)cs1)c1cccn1Cc1ccncc1] |
| E12 | 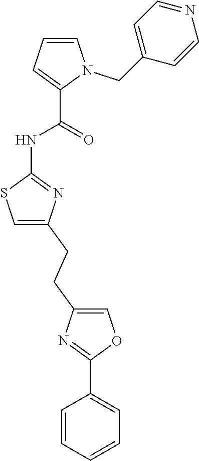 [SMILES: O=C(Nc1nc(CCc2coc(-c3ccccc3)n2)cs1)c1cccn1Cc1ccncc1] |
| E13 | 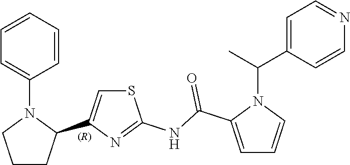 |
| E14 | 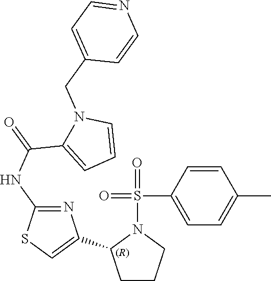 |
| E15 | 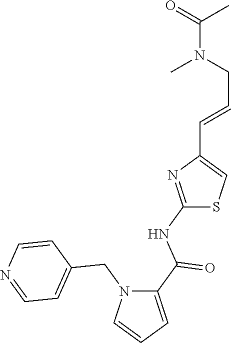 [SMILES: CC(=O)N(C)C/C=C/c1csc(NC(=O)c2cccn2Cc2ccncc2)n1] |
| E16 | 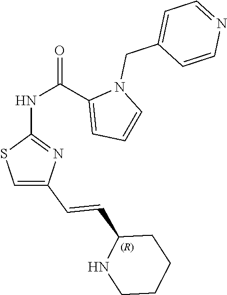 |
| E17 | 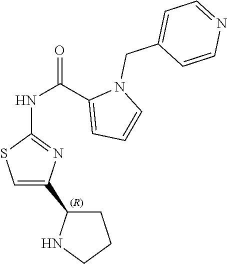 |
| E18 | 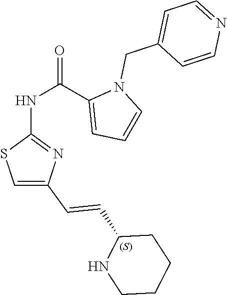 |
| E19 | 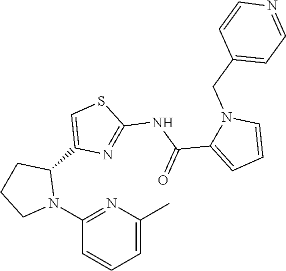 [SMILES: Cc1cccc(N2CCC[C@@H]2c2csc(NC(=O)c3cccn3Cc3ccncc3)n2)n1] |
| E20 | 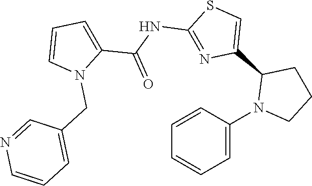 |
| E21 | 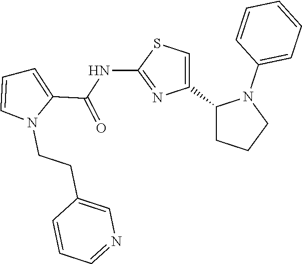 [SMILES: O=C(Nc1nc([C@H]2CCCN2c2ccccc2)cs1)c1cccn1CCc1cccnc1] |
| E22 | 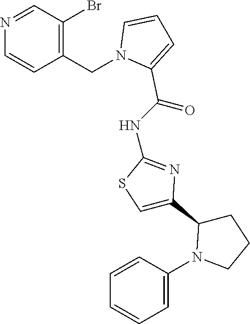 [SMILES: O=C(Nc1nc([C@H]2CCCN2c2ccccc2)cs1)c1cccn1Cc1ccncc1Br] |
| E23 | 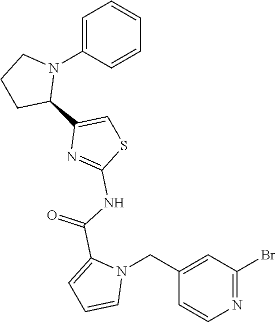 [SMILES: O=C(Nc1nc([C@H]2CCCN2c2ccccc2)cs1)c1cccn1Cc1ccnc(Br)c1] |
| E24 | 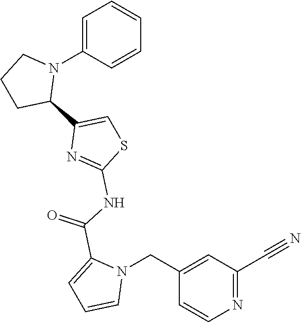 [SMILES: N#Cc1cc(Cn2cccc2C(=O)Nc2nc([C@H]3CCCN3c3ccccc3)cs2)ccn1] |
| E25 | 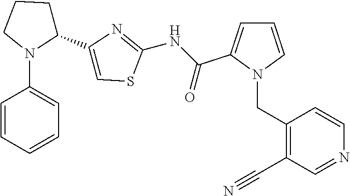 [SMILES: N#Cc1cnccc1Cn1cccc1C(=O)Nc1nc([C@H]2CCCN2c2ccccc2)cs1] |
| E26 | 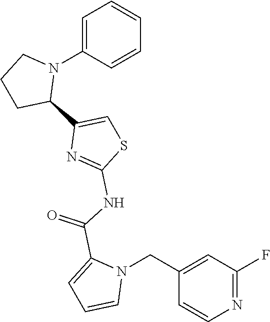 [SMILES: O=C(Nc1nc([C@H]2CCCN2c2ccccc2)cs1)c1cccn1Cc1ccnc(F)c1] |
| E27 | 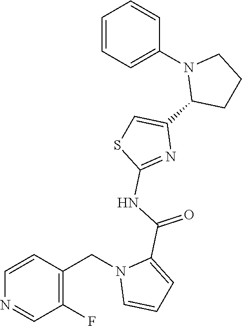 [SMILES: O=C(Nc1nc([C@H]2CCCN2c2ccccc2)cs1)c1cccn1Cc1ccncc1F] |
| E28 | 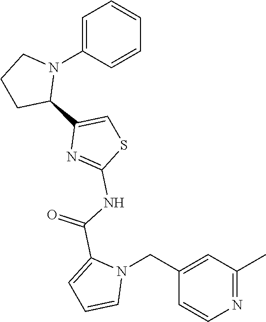 [SMILES: Cc1cc(Cn2cccc2C(=O)Nc2nc([C@H]3CCCN3c3ccccc3)cs2)ccn1] |
| E29 | 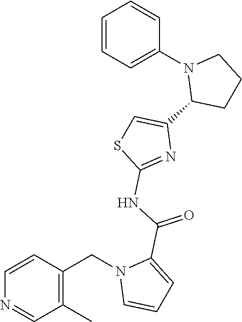 [SMILES: Cc1cnccc1Cn1cccc1C(=O)Nc1nc([C@H]2CCCN2c2ccccc2)cs1] |
| E30 | 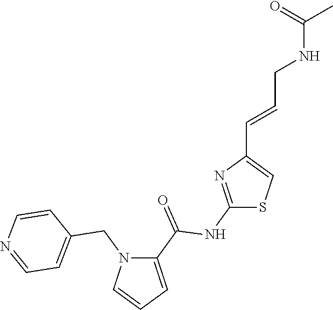 [SMILES: CC(=O)NC/C=C/c1csc(NC(=O)c2cccn2Cc2ccncc2)n1] |
| E31 | 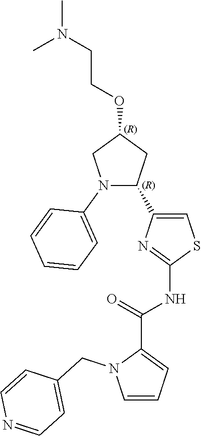 |
| E32 | 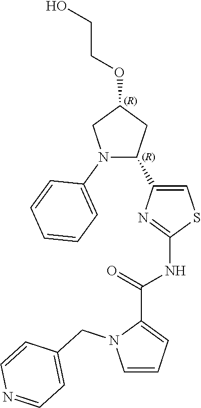 |
| E33 | 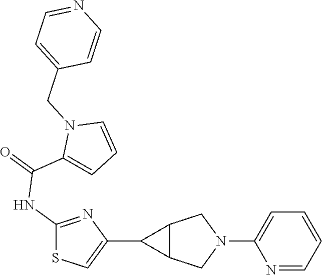 [SMILES: O=C(Nc1nc(C2C3CN(c4ccccn4)CC32)cs1)c1cccn1Cc1ccncc1] |
| E34 | 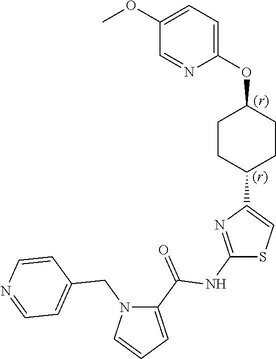 [SMILES: COc1ccc(O[C@H]2CC[C@H](c3csc(NC(=O)c4cccn4Cc4ccncc4)n3)CC2)nc1] |
| E35 | 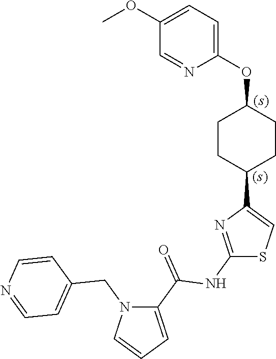 [SMILES: COc1ccc(O[C@H]2CC[C@@H](c3csc(NC(=O)c4cccn4Cc4ccncc4)n3)CC2)nc1] |
| E36 | 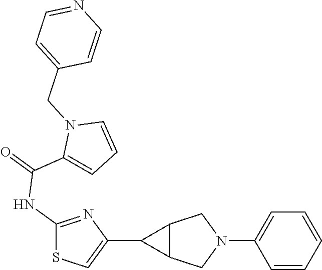 [SMILES: O=C(Nc1nc(C2C3CN(c4ccccc4)CC32)cs1)c1cccn1Cc1ccncc1] |
| E37 | 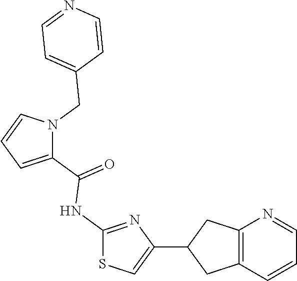 [SMILES: O=C(Nc1nc(C2Cc3cccnc3C2)cs1)c1cccn1Cc1ccncc1] |
| E38 | 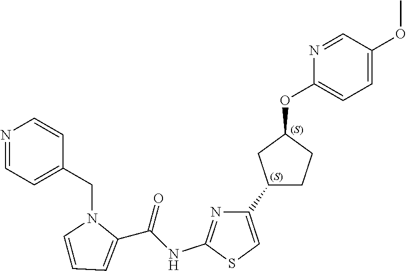 |
| E39 | 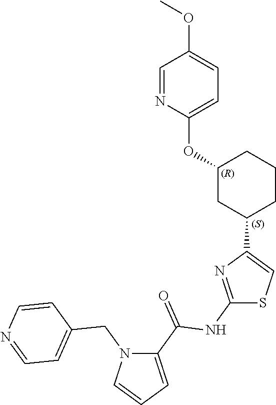 |
| E40 | 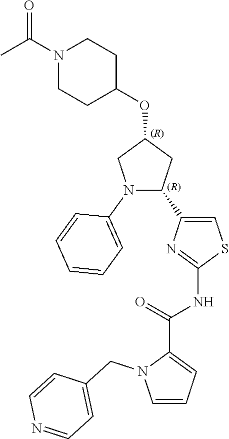 |
| E41 | 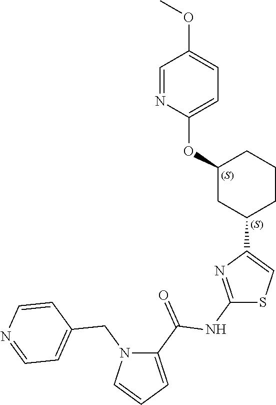 |
| E42 | 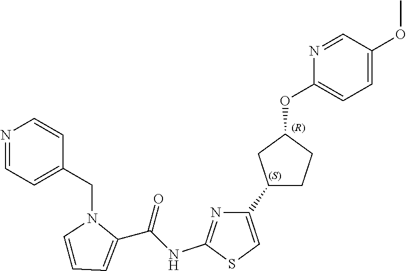 |
| E43 | 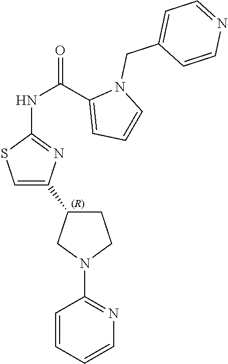 |
| E44 | 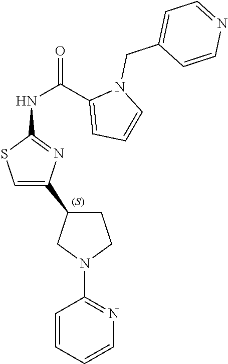 |
| E45 | 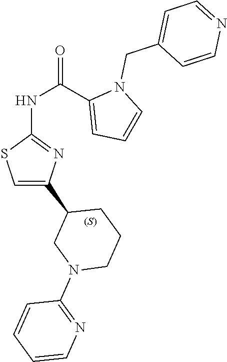 |
| E46 | 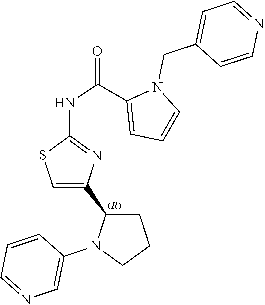 |
| E47 | 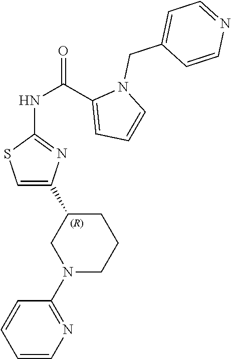 |
| E48 | 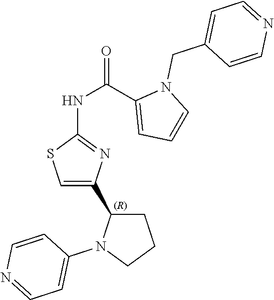 |
| E49 | 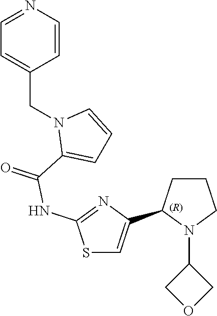 |
| E50 | 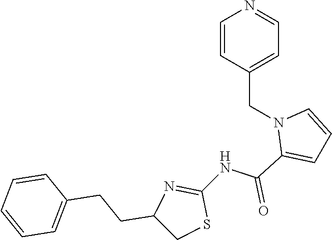 [SMILES: O=C(NC1=NC(CCc2ccccc2)CS1)c1cccn1Cc1ccncc1] |
| E51 | 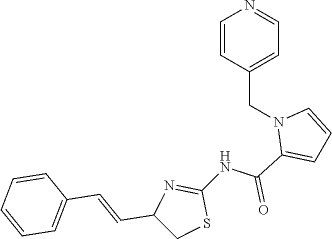 [SMILES: O=C(NC1=NC(/C=C/c2ccccc2)CS1)c1cccn1Cc1ccncc1] |
| E52 | 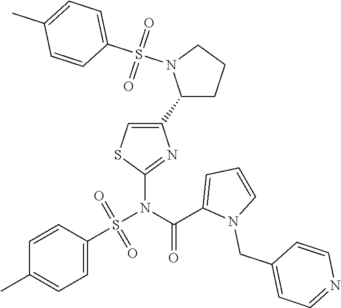 [SMILES: Cc1ccc(S(=O)(=O)N(C(=O)c2cccn2Cc2ccncc2)c2nc([C@H]3CCCN3S(=O)(=O)c3ccc(C)cc3)cs2)cc1] |
| E53 | 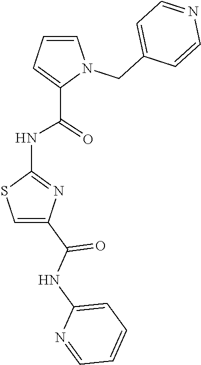 [SMILES: O=C(Nc1ccccn1)c1csc(NC(=O)c2cccn2Cc2ccncc2)n1] |
| E54 | 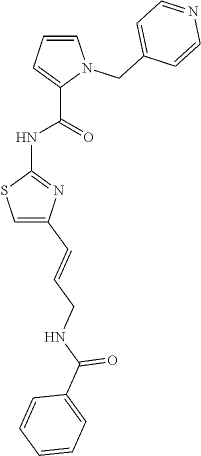 [SMILES: O=C(NC/C=C/c1csc(NC(=O)c2cccn2Cc2ccncc2)n1)c1ccccc1] |
| E55 | 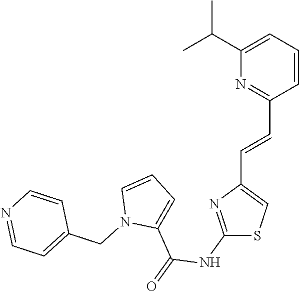 [SMILES: CC(C)c1cccc(/C=C/c2csc(NC(=O)c3cccn3Cc3ccncc3)n2)n1] |
| E56 | 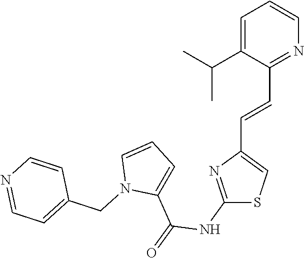 [SMILES: CC(C)c1cccnc1/C=C/c1csc(NC(=O)c2cccn2Cc2ccncc2)n1] |
| E57 | 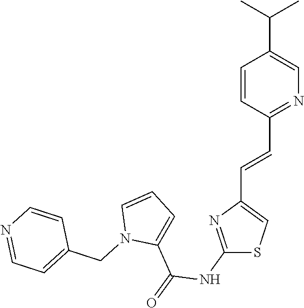 [SMILES: CC(C)c1ccc(/C=C/c2csc(NC(=O)c3cccn3Cc3ccncc3)n2)nc1] |
| E58 | 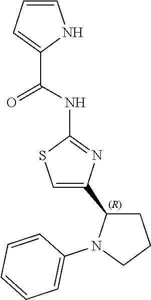 |
| E59 | 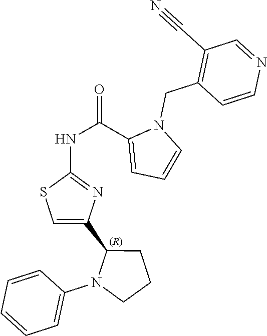 |
| E60 | 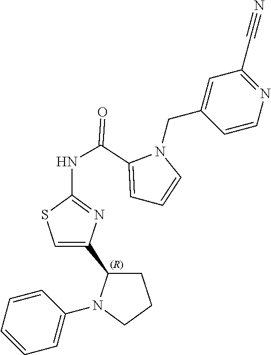 |
| E61 | 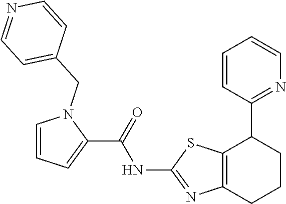 [SMILES: O=C(Nc1nc2c(s1)C(c1ccccn1)CCC2)c1cccn1Cc1ccncc1] |
| E62 | 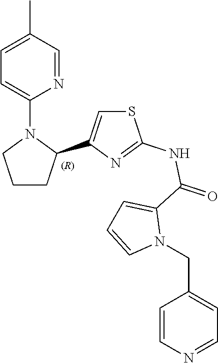 |
| E63 | 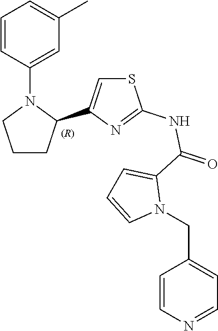 |
| E64 | 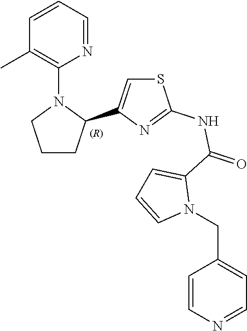 |
| E65 | 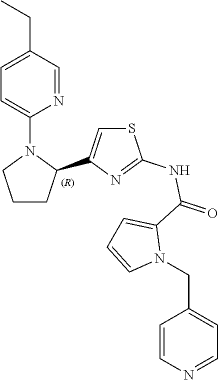 |
| E66 | 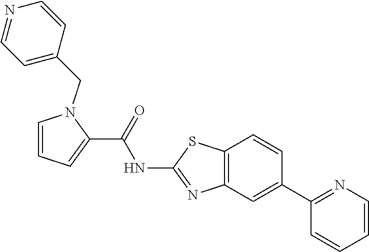 [SMILES: O=C(Nc1nc2cc(-c3ccccn3)ccc2s1)c1cccn1Cc1ccncc1] |
| E67 | 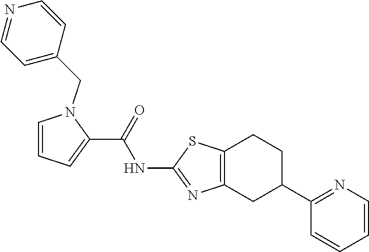 [SMILES: O=C(Nc1nc2c(s1)CCC(c1ccccn1)C2)c1cccn1Cc1ccncc1] |
| E68 | 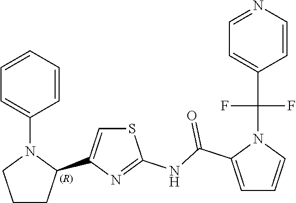 |
| E69 | 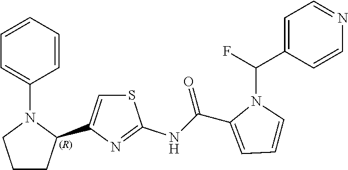 |
| E70 | 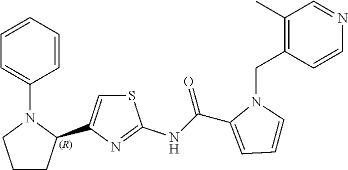 |
| E71 | 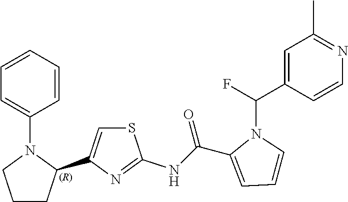 |

As used herein, reference to an element, whether by description or chemical structure, encompasses all isotopes of that element unless otherwise described. By way of example, the term “hydrogen” or “H” in a chemical structure as used herein is understood to encompass, for example, not only ¹H, but also deuterium (²H), tritium (³H), and mixtures thereof unless otherwise denoted by use of a specific isotope. Other specific non-limiting examples of elements for which isotopes are encompassed include carbon, phosphorous, idodine, and fluorine.

Without being bound by any particular theory, the compounds described herein inhibit protein secretion by binding to and disabling components of the translocon, including but not limited to Sec61, and in some cases, disrupting in a sequence specific fashion interactions between the nascent signaling sequence of translated proteins with components of the translocon including but not limited to Sec61.

The compounds described herein can advantageously inhibit the secretion of a protein of interest with an IC50 of up to 5 μM, or up to 3 μM, or up to 1 μM. In various cases, the compounds disclosed herein can inhibit the secretion of TNFα with an IC50 of up to 5 μM, or up to 3 μM, or up to 1 μM. In various cases, the compounds disclosed herein can inhibit the secretion of Her3 with an IC50 of up to 5 μM, or up to 3 μM, or up to 1 μM. In some cases, the compounds disclosed herein can inhibit the secretion of IL2 with an IC50 of up to 5 μM, or up to 3 μM, or up to 1 μM. In various cases, the compounds disclosed herein can inhibit the secretion of PD-1 with an IC50 of up to 5 μM, or up to 3 μM, or up to 1 μM.

### Chemical Definitions

The compounds disclosed herein include all pharmaceutically acceptable isotopically-labeled compounds wherein one or more atoms of the compounds disclosed herein are replaced by atoms having the same atomic number, but an atomic mass or mass number different from the atomic mass or mass number usually found in nature, examples of which include isotopes of hydrogen, such as ²H and ³H. In some cases, one or more hydrogen atoms of the compounds disclosed herein are specifically deuterium (²H).

As used herein, the term “alkyl” refers to straight chained and branched saturated hydrocarbon groups containing one to thirty carbon atoms, for example, one to twenty carbon atoms, or one to ten carbon atoms. The term Cₙ means the alkyl group has “n” carbon atoms. For example, C₄alkyl refers to an alkyl group that has 4 carbon atoms. C₁₋₆alkyl refers to an alkyl group having a number of carbon atoms encompassing the entire range (i.e., 1 to 6 carbon atoms), as well as all subgroups (e.g., 1-5, 2-5, 1-4, 2-5, 1, 2, 3, 4, 5, and 6 carbon atoms). Nonlimiting examples of alkyl groups include, methyl, ethyl, n-propyl, isopropyl, n-butyl, sec-butyl (2-methylpropyl), and t-butyl (1,1-dimethylethyl). Unless otherwise indicated, an alkyl group can be an unsubstituted alkyl group or a substituted alkyl group.

As used herein, the term “alkylene” refers to a bivalent saturated aliphatic radical. The term Cₙ means the alkylene group has “n” carbon atoms. For example, C₁₋₆alkylene refers to an alkylene group having a number of carbon atoms encompassing the entire range, as well as all subgroups, as previously described for “alkyl” groups.

As used herein, the term “alkene” or “alkenyl” is defined identically as “alkyl” except for containing at least one carbon-carbon double bond, and having two to thirty carbon atoms, for example, two to twenty carbon atoms, or two to ten carbon atoms. The term Cₙ means the alkenyl group has “n” carbon atoms. For example, C₄alkenyl refers to an alkenyl group that has 4 carbon atoms. C₂₋₇alkenyl refers to an alkenyl group having a number of carbon atoms encompassing the entire range (i.e., 2 to 7 carbon atoms), as well as all subgroups (e.g., 2-6, 2-5, 3-6, 2, 3, 4, 5, 6, and 7 carbon atoms). Specifically contemplated alkenyl groups include ethenyl, 1-propenyl, 2-propenyl, and butenyl. Unless otherwise indicated, an alkenyl group can be an unsubstituted alkenyl group or a substituted alkenyl group. Unless otherwise indicated, an alkenyl group can be a cis-alkenyl or trans-alkenyl.

As used herein, the term “alkyne” or “alkynyl” is defined identically as “alkyl” except for containing at least one carbon-carbon triple bond, and having two to thirty carbon atoms, for example, two to twenty carbon atoms, or two to ten carbon atoms. The term Cₙ means the alkynyl group has “n” carbon atoms. For example, C₄alkynyl refers to an alkynyl group that has 4 carbon atoms. C₂₋₇alkynyl refers to an alkynyl group having a number of carbon atoms encompassing the entire range (i.e., 2 to 7 carbon atoms), as well as all subgroups (e.g., 2-6, 2-5, 3-6, 2, 3, 4, 5, 6, and 7 carbon atoms). Specifically contemplated alkynyl groups include ethynyl, 1-propynyl, 2-propynyl, and butynyl. Unless otherwise indicated, an alkynyl group can be an unsubstituted alkynyl group or a substituted alkynyl group.

As used herein, the term “carbocycle” refers to an aromatic or nonaromatic (i.e., fully or partially saturated) ring in which each atom of the ring is carbon. A carbocycle can include, for example, from three to ten carbon atoms, four to eight carbon atoms, or five to six carbon atoms. As used herein, the term “carbocycle” also includes polycyclic ring systems having two or more cyclic rings in which two or more carbons are common to two adjoining rings wherein at least one of the rings is carbocyclic, e.g., the other cyclic rings can be cycloalkyls, cycloalkenyls, aryls, heteroaryls, and/or heterocycles.

As used herein, the term “cycloalkyl” specifically refers to a non-aromatic carbocycle. The term Cₙ means the cycloalkyl group has “n” carbon atoms. For example, C₅ cycloalkyl refers to a cycloalkyl group that has 5 carbon atoms in the ring. C₅₋₈ cycloalkyl refers to cycloalkyl groups having a number of carbon atoms encompassing the entire range (i.e., 5 to 10 carbon atoms), as well as all subgroups (e.g., 5-10, 5-9, 5-8, 5-6, 6-8, 7-8, 5-7, 5, 6, 7, 8, 9 and 10 carbon atoms). Nonlimiting examples of cycloalkyl groups include cyclopropyl, cyclobutyl, cyclopentyl, cyclohexyl, cycloheptyl, and cyclooctyl. Unless otherwise indicated, a cycloalkyl group can be an unsubstituted cycloalkyl group or a substituted cycloalkyl group.

As used herein, the term “aryl” refers to an aromatic carbocycle, and can be monocyclic or polycyclic (e.g., fused bicyclic and fused tricyclic) carbocyclic aromatic ring systems. Examples of aryl groups include, but are not limited to, phenyl, naphthyl, tetrahydronaphthyl, phenanthrenyl, biphenylenyl, indanyl, indenyl, anthracenyl, fluorenyl, tetralinyl. Unless otherwise indicated, an aryl group can be an unsubstituted aryl group or a substituted aryl group.

As used herein, the term “heterocycle” is defined similarly as carbocycle, except the ring contains one to four heteroatoms independently selected from oxygen, nitrogen, and sulfur. For example, a heterocycle can be a 3-10 membered aromatic or non-aromatic ring having 1 or 2 heteroatoms selected from N, O, and S. As another example, a heterocycle can be a 5-6 membered ring having 1 or 2 ring heteroatoms selected from N, O, and S. Nonlimiting examples of heterocycle groups include piperdine, tetrahydrofuran, tetrahydropyran, dihydrofuran, morpholine, oxazepaneyl, thiazole, pyrrole, and pyridine.

Carbocyclic and heterocyclic groups can be saturated or partially unsaturated ring systems optionally substituted with, for example, one to three groups, independently selected alkyl, alkoxy, alkyleneOH, C(O)NH₂, NH₂, oxo (═O), aryl, haloalkyl, haloalkoxy, C(O)-alkyl, SO₂alkyl, halo, OH, NHC₁₋₃alkylene-aryl, OC₁₋₃alkylene-aryl, C₁₋₃alkylene-aryl, and C₃₋₆heterocycloalkyl having 1-3 heteroatoms selected from N, O, and S. Heterocyclic groups optionally can be further N-substituted as described herein. Other substituents contemplated for the disclosed rings is provided elsewhere in this disclosure.

As used herein, the term “heteroaryl” refers to an aromatic heterocycle, and can be monocyclic or polycyclic (e.g., fused bicyclic and fused tricyclic) aromatic ring systems, wherein one to four-ring atoms are selected from oxygen, nitrogen, or sulfur, and the remaining ring atoms are carbon, said ring system being joined to the remainder of the molecule by any of the ring atoms. Nonlimiting examples of heteroaryl groups include, but are not limited to, pyridyl, pyridazinyl, pyrazinyl, pyrimidinyl, pyrrolyl, pyrazolyl, imidazolyl, thiazolyl, tetrazolyl, oxazolyl, isooxazolyl, thiadiazolyl, oxadiazolyl, furanyl, thienyl, quinolinyl, isoquinolinyl, benzoxazolyl, benzimidazolyl, benzofuranyl, benzothiazolyl, triazinyl, triazolyl, purinyl, pyrazinyl, purinyl, indolinyl, phthalzinyl, indazolyl, quinolinyl, isoquinolinyl, cinnolinyl, quinazolinyl, naphthyridinyl, pyridopyridinyl, indolyl, 3H-indolyl, pteridinyl, and quinooxalinyl. Unless otherwise indicated, a heteroaryl group can be an unsubstituted heteroaryl group or a substituted heteroaryl group.

As used herein, the term “hydroxy” or “hydroxyl” as used herein refers to an “—OH” group. Accordingly, a “hydroxyalkyl” refers to an alkyl group substituted with one or more —OH groups.

As used herein, the term “alkoxy” or “alkoxyl” refers to a “—O-alkyl” group.

As used herein, the term “halo” is defined as fluoro, chloro, bromo, and iodo. Accordingly, a “haloalkyl” refers to an alkyl group substituted with one or more halo atoms. A “haloalkoxy” refers to an alkoxy group that is substituted with one or more halo atoms.

A “substituted” functional group (e.g., a substituted alkyl, cycloalkyl, aryl, or heteroaryl) is a functional group having at least one hydrogen radical that is substituted with a non-hydrogen radical (i.e., a substituent). Examples of non-hydrogen radicals (or substituents) include, but are not limited to, alkyl, cycloalkyl, alkenyl, cycloalkenyl, alkynyl, ether, aryl, O-alkylene aryl, N-alkylene aryl, alkylene aryl, heteroaryl, heterocycloalkyl, hydroxy, hydroxyalkyl, haloalkoxy, amido, oxy (or oxo), alkoxy, ester, thioester, acyl, carboxyl, cyano, nitro, amino, sulfhydryl, and halo. When a substituted alkyl group includes more than one non-hydrogen radical, the substituents can be bound to the same carbon or two or more different carbon atoms.

The chemical structures having one or more stereocenters depicted with dashed and bold wedged bonds (i.e., [symbol] and [symbol] ) are meant to indicate absolute stereochemistry of the stereocenter(s) present in the chemical structure. Bonds symbolized by a simple line do not indicate a stereo-preference. Bonds symbolized by dashed or bold straight bonds (i.e., [symbol] and [symbol] ) are meant to indicate a relative stereochemistry of the stereocenter(s) present in the chemical structure. Unless otherwise indicated to the contrary, chemical structures that include one or more stereocenters which are illustrated herein without indicating absolute or relative stereochemistry, encompass all possible stereoisomeric forms of the compound (e.g., diastereomers, enantiomers) and mixtures thereof. Structures with a single bold or dashed wedged line, and at least one additional simple line, encompass a single enantiomeric series of all possible diastereomers. Similarly, the chemical structures having alkenyl groups are meant to encompass both cis and trans orientations, or when substituted, E- and Z-isomers of the chemical structure.

### Synthesis of Protein Secretion Inhibitors

The compounds provided herein can be synthesized using conventional techniques readily available starting materials known to those skilled in the art. In general, the compounds provided herein are conveniently obtained via standard organic chemistry synthesis methods.

Although not limited to any one or several sources, classic texts such as Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3^{rd} edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

In general, the compounds of the disclosure can be synthesized in line with the examples shown below. For example, the compounds can be prepared by alkylation of the appropriate amine having a carboxyl group, with appropriate protecting groups as necessary. The intermediate can be saponified, for example, to expose a reactive carboxylate. Then, amide coupling between the appropriate amine and the free carboxylate can occur.

The amine for the amide coupling noted above can be prepared via known synthetic techniques using appropriate starting materials and protecting groups, as necessary.

Further modifications can be performed, e.g., to introduce additional substituents such as halo groups or alkyl groups.

### Methods of Use

The compounds disclosed herein can inhibit protein secretion of a protein of interest. The compounds disclosed herein can interfere with the Sec61 protein secretion machinery of a cell. In some cases, a compound as disclosed herein inhibits secretion of one or more of TNFα, IL2, Her3, and PD-1, or each of TNFα, IL2, Her3, and PD-1. Protein secretion activity can be assessed in a manner as described in the Examples section below.

As used herein, the term “inhibitor” is meant to describe a compound that blocks or reduces an activity of a pharmacological target (for example, a compound that inhibits Sec61 function in the protein secretion pathway). An inhibitor can act with competitive, uncompetitive, or noncompetitive inhibition. An inhibitor can bind reversibly or irreversibly, and therefore, the term includes compounds that are suicide substrates of a protein or enzyme. An inhibitor can modify one or more sites on or near the active site of the protein, or it can cause a conformational change elsewhere on the enzyme. The term inhibitor is used more broadly herein than scientific literature so as to also encompass other classes of pharmacologically or therapeutically useful agents, such as agonists, antagonists, stimulants, co-factors, and the like.

Thus, provided herein are methods of inhibiting protein secretion in a cell. In these methods, a cell is contacted with a compound described herein, or pharmaceutical composition thereof, in an amount effective to inhibit secretion of the protein of interest. In some embodiments, the cell is contacted in vitro. In various embodiments, the cell is contacted in vivo. In various embodiments, the contacting includes administering the compound or pharmaceutical composition to a subject.

The biological consequences of Sec61 inhibition are numerous. For example, Sec61 inhibition has been suggested for the treatment or prevention of inflammation and/or cancer in a subject. Therefore, pharmaceutical compositions for Sec61 specific compounds, provide a means of administering a drug to a subject and treating these conditions. As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound of the disclosure to an individual in need of such treatment. Within the meaning of the disclosure, “treatment” also includes relapse prophylaxis or phase prophylaxis, as well as the treatment of acute or chronic signs, symptoms and/or malfunctions. The treatment can be orientated symptomatically, for example, to suppress symptoms. It can be effected over a short period, be oriented over a medium term, or can be a long-term treatment, for example within the context of a maintenance therapy. As used herein, the terms “prevent,” “preventing,” “prevention,” are art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. As used herein, the terms “patient” and “subject” may be used interchangeably and mean animals, such as dogs, cats, cows, horses, and sheep (i.e., non-human animals) and humans. Particular patients are mammals (e.g., humans). The term patient includes males and females.

Inhibition of Sec61-mediated secretion of inflammatory proteins (e.g., TNFα) can disrupt inflammation signaling. Thus, provided herein is a method of treating inflammation in a subject by administering to the subject a therapeutically effective amount of a compound described herein.

Further, the viability of cancer cells relies upon increased protein secretion into the ER for survival. Therefore, non-selective or partially selective inhibition of Sec61 mediated protein secretion may inhibit tumor growth. Alternatively, in the immune-oncology setting, selective secretion inhibitors of known secreted immune checkpoints proteins (e.g., PD-1, TIM-3, LAG3, etc.) can result in activation of the immune system to against various cancers.

Accordingly, also provided herein are methods of treating cancer in a subject by administering to the subject a therapeutically effective amount of a compound described herein or a pharmaceutically acceptable salt thereof. Specifically contemplated cancers that can be treated using the compounds and compositions described herein include, but are not limited to melanoma, multiple myeloma, prostate, lung, non small cell lung carconimoa (NSCLC), squamous cell carcinoma, leukemia, acute myelogenous leukemia, chronic myelogenous leukemia, lymphoma, NPM/ALK-transformed anaplastic large cell lymphoma, renal cell carcinoma, rhabdomyosarcoma, ovarian cancer, endometrial cancer, small cell carcinoma, adenocarcinoma, gastric carcinoma, hepatocellular carcinoma, pancreatic cancer, thyroid carcinoma, anaplastic large cell lymphoma, hemangioma, head and neck cancer, bladder, and colorectal cancers.

The compounds described herein are also contemplated to be used in the prevention and/or treatment of a multitude of diseases including, but not limited to, proliferative diseases, neurotoxic/degenerative diseases, ischemic conditions, autoimmune and autoinflammatory disorders, inflammation, immune-related diseases, HIV, cancers, organ graft rejection, septic shock, viral and parasitic infections, conditions associated with acidosis, macular degeneration, pulmonary conditions, muscle wasting diseases, fibrotic diseases, bone and hair growth diseases.

Examples of proliferative diseases or conditions include diabetic retinopathy, macular degeneration, diabetic nephropathy, glomerulosclerosis, IgA nephropathy, cirrhosis, biliary atresia, congestive heart failure, scleroderma, radiation-induced fibrosis, and lung fibrosis (idiopathic pulmonary fibrosis, collagen vascular disease, sarcoidosis, interstitial lung diseases and extrinsic lung disorders).

Inflammatory diseases include acute (e.g., bronchitis, conjunctivitis, myocarditis, pancreatitis) and chronic conditions (e.g., chronic cholecstitis, bronchiectasis, aortic valve stenosis, restenosis, psoriasis and arthritis), along with conditions associated with inflammation such as fibrosis, infection and ischemia.

Immunodeficiency disorders occur when a part of the immune system is not working properly or is not present. They can affect B lymophyctes, T lymphocytes, or phagocytes and be either inherited (e.g., IgA deficiency, severe combined immunodeficiency (SCID), thymic dysplasia and chronic granulomatous) or acquired (e.g., acquired immunodeficiency syndrome (AIDS), human immunodeficiency virus (HIV) and drug-induced immunodeficiencies). Immune-related conditions include allergic disorders such as allergies, asthma and atopic dermatitis like eczema. Other examples of such immune-related conditions include lupus, rheumatoid arthritis, scleroderma, ankylosing spondylitis, dermatomyositis, psoriasis, multiple sclerosis and inflammatory bowel disease (such as ulcerative colitis and Crohn's disease).

Tissue/organ graft rejection occurs when the immune system mistakenly attacks the cells being introduced to the host's body. Graft versus host disease (GVHD), resulting from allogenic transplantation, arises when the T cells from the donor tissue go on the offensive and attack the host's tissues. In all three circumstances, autoimmune disease, transplant rejection and GVHD, modulating the immune system by treating the subject with a compound or composition of the disclosure could be beneficial.

Also provided herein are methods of treating an autoimmune disease in a patient comprising administering a therapeutically effective amount of the compound described herein. An “autoimmune disease” as used herein is a disease or disorder arising from and directed against an individual's own tissues. Examples of autoimmune diseases include, but are not limited to, inflammatory responses such as inflammatory skin diseases including psoriasis and dermatitis (e.g., atopic dermatitis); systemic scleroderma and sclerosis; responses associated with inflammatory bowel disease (such as Crohn's disease and ulcerative colitis); respiratory distress syndrome (including adult respiratory distress syndrome (ARDS)); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE); diabetes mellitus (e.g., Type I diabetes mellitus or insulin dependent diabetes mellitus); multiple sclerosis; Reynaud's syndrome; autoimmune thyroiditis; allergic encephalomyelitis; Sjorgen's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia. Compounds provided herein may be useful for the treatment of conditions associated with inflammation, including, but not limited to COPD, psoriasis, asthma, bronchitis, emphysema, and cystic fibrosis.

Also provided herein is the use of a compound as disclosed herein for the treatment of neurodegenerative diseases. Neurodegenerative diseases and conditions includes, but not limited to, stroke, ischemic damage to the nervous system, neural trauma (e.g., percussive brain damage, spinal cord injury, and traumatic damage to the nervous system), multiple sclerosis and other immune-mediated neuropathies (e.g., Guillain-Barre syndrome and its variants, acute motor axonal neuropathy, acute inflammatory demyelinating polyneuropathy, and Fisher Syndrome), HIV/AIDS dementia complex, axonomy, diabetic neuropathy, Parkinson's disease, Huntington's disease, multiple sclerosis, bacterial, parasitic, fungal, and viral meningitis, encephalitis, vascular dementia, multi-infarct dementia, Lewy body dementia, frontal lobe dementia such as Pick's disease, subcortical dementias (such as Huntington or progressive supranuclear palsy), focal cortical atrophy syndromes (such as primary aphasia), metabolic-toxic dementias (such as chronic hypothyroidism or B12 deficiency), and dementias caused by infections (such as syphilis or chronic meningitis).

Further guidance for using compounds and compositions described for inhibiting protein secretion can be found in the Examples section, below.

### Pharmaceutical Compositions and Administration

Provided herein is disclosure for the manufacture and use of pharmaceutical compositions, which include one or more of the compounds as disclosed herein. Also included are the pharmaceutical compositions themselves. Pharmaceutical compositions typically include a pharmaceutically acceptable carrier. Thus, provided herein are pharmaceutical compositions that include a compound described herein and one or more pharmaceutically acceptable carriers.

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition, or vehicle, such as a liquid or solid filler, diluent, excipient, solvent or encapsulating material. As used herein the language “pharmaceutically acceptable carrier” includes buffer, sterile water for injection, solvents, dispersion media, coatings, antibacterial and antifungal agents, isotonic and absorption delaying agents, and the like, compatible with pharmaceutical administration. Each carrier must be “acceptable” in the sense of being compatible with the other ingredients of the composition and not injurious to the patient. Some examples of materials which can serve as pharmaceutically acceptable carriers include: (1) sugars, such as lactose, glucose, and sucrose; (2) starches, such as corn starch, potato starch, and substituted or unsubstituted β-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions. In certain embodiments, pharmaceutical compositions provided herein are non-pyrogenic, i.e., do not induce significant temperature elevations when administered to a patient.

The term “pharmaceutically acceptable salt” refers to the relatively non-toxic, inorganic and organic acid addition salts of a compound provided herein. These salts can be prepared in situ during the final isolation and purification of a compound provided herein, or by separately reacting the compound in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66: 1-19.)

In some embodiments, a compound provided herein may contain one or more acidic functional groups and, thus, is capable of forming pharmaceutically acceptable salts with pharmaceutically acceptable bases. The term “pharmaceutically acceptable salts” in these instances refers to the relatively non-toxic inorganic and organic base addition salts of a compound provided herein. These salts can likewise be prepared in situ during the final isolation and purification of the compound, or by separately reacting the purified compound in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

A pharmaceutical composition may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include tonicity-adjusting agents, such as sugars and the like into the compositions. In addition, prolonged absorption of the injectable pharmaceutical form may be brought about by the inclusion of agents which delay absorption such as aluminum monostearate and gelatin.

In some cases, in order to prolong the effect of one or more compounds provided herein, it is desirable to slow the absorption of the compound from subcutaneous or intramuscular injection. For example, delayed absorption of a parenterally administered compound can be accomplished by dissolving or suspending the compound in an oil vehicle.

Compositions prepared as described herein can be administered in various forms, depending on the disorder to be treated and the age, condition, and body weight of the patient, as is well known in the art. For example, where the compositions are to be administered orally, they may be formulated as tablets, capsules, granules, powders, or syrups; or for parenteral administration, they may be formulated as injections (intravenous, intramuscular, or subcutaneous), drop infusion preparations, or suppositories. For application by the ophthalmic mucous membrane route, they may be formulated as eye drops or eye ointments. These compositions can be prepared by conventional means in conjunction with the methods described herein, and, if desired, the active ingredient may be mixed with any conventional additive or excipient, such as a binder, a disintegrating agent, a lubricant, a corrigent, a solubilizing agent, a suspension aid, an emulsifying agent, or a coating agent.

Compositions suitable for oral administration may be in the form of capsules (e.g., gelatin capsules), cachets, pills, tablets, lozenges (using a flavored basis, usually sucrose and acacia or tragacanth), powders, troches, granules, or as a solution or a suspension in an aqueous or non-aqueous liquid, or as an oil-in-water or water-in-oil liquid emulsion, or as an elixir or syrup, or as pastilles (using an inert matrix, such as gelatin and glycerin, or sucrose and acacia) and/or as mouthwashes, and the like, each containing a predetermined amount of a compound provided herein as an active ingredient. A composition may also be administered as a bolus, electuary, or paste. Oral compositions generally include an inert diluent or an edible carrier.

Pharmaceutically compatible binding agents, and/or adjuvant materials can be included as part of an oral composition. In solid dosage forms for oral administration (capsules, tablets, pills, dragees, powders, granules, and the like), the active ingredient can be mixed with one or more pharmaceutically acceptable carriers, such as sodium citrate or dicalcium phosphate, and/or any of the following: (1) fillers or extenders, such as starches, cyclodextrins, lactose, sucrose, saccharin, glucose, mannitol, and/or silicic acid; (2) binders, such as, for example, carboxymethylcellulose, microcrystalline cellulose, gum tragacanth, alginates, gelatin, polyvinyl pyrrolidone, sucrose, and/or acacia; (3) humectants, such as glycerol; (4) disintegrating agents, such as agar-agar, calcium carbonate, potato, corn, or tapioca starch, alginic acid, Primogel, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, Sterotes, solid polyethylene glycols, sodium lauryl sulfate, and mixtures thereof; (10) a glidant, such as colloidal silicon dioxide; (11) coloring agents; and (12) a flavoring agent such as peppermint, methyl salicylate, or orange flavoring. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols, and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of a powdered compound moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, microspheres, and/or nanoparticles. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active compound(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions suitable for parenteral administration can include one or more compounds provided herein in combination with one or more pharmaceutically acceptable sterile aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the composition isotonic with the blood of the intended recipient or suspending or thickening agents.

Examples of suitable aqueous and nonaqueous carriers which may be employed in the pharmaceutical compositions provided herein include water for injection (e.g., sterile water for injection), bacteriostatic water, ethanol, polyols (such as glycerol, propylene glycol, polyethylene glycol such as liquid polyethylene glycol, and the like), sterile buffer (such as citrate buffer), and suitable mixtures thereof, vegetable oils, such as olive oil, injectable organic esters, such as ethyl oleate, and Cremophor EL™ (BASF, Parsippany, N.J.). In all cases, the composition must be sterile and should be fluid to the extent that easy syringability exists. Proper fluidity can be maintained, for example, by the use of coating materials, such as lecithin, by the maintenance of the required particle size in the case of dispersions, and by the use of surfactants.

The composition should be stable under the conditions of manufacture and storage and must be preserved against the contaminating action of microorganisms such as bacteria and fungi. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the methods of preparation are freeze-drying (lyophilization), which yields a powder of the active ingredient plus any additional desired ingredient from a previously sterile-filtered solution thereof.

Injectable depot forms can be made by forming microencapsule or nanoencapsule matrices of a compound provided herein in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable compositions are also prepared by entrapping the drug in liposomes, microemulsions or nanoemulsions, which are compatible with body tissue.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798. Additionally, intranasal delivery can be accomplished, as described in, inter alia, Hamajima et al., Clin. Immunol. Immunopathol., 88(2), 205-10 (1998). Liposomes (e.g., as described in U.S. Pat. No. 6,472,375, which is incorporated herein by reference in its entirety), microencapsulation and nanoencapsulation can also be used. Biodegradable targetable microparticle delivery systems or biodegradable targetable nanoparticle delivery systems can also be used (e.g., as described in U.S. Pat. No. 6,471,996, which is incorporated herein by reference in its entirety).

Systemic administration of a therapeutic compound as described herein can also be by transmucosal or transdermal means. Dosage forms for the topical or transdermal administration of a compound provided herein include powders, sprays, ointments, pastes, creams, lotions, gels, solutions, patches, and inhalants. The active component may be mixed under sterile conditions with a pharmaceutically acceptable carrier, and with any preservatives, buffers, or propellants which may be required. For transmucosal or transdermal administration, penetrants appropriate to the barrier to be permeated are used in the composition. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The ointments, pastes, creams, and gels may contain, in addition to one or more compounds provided herein, excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to a compound provided herein, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

A compound provided herein can be administered by aerosol. This is accomplished by preparing an aqueous aerosol, liposomal preparation, or solid particles containing a compound or composition provided herein. A nonaqueous (e.g., fluorocarbon propellant) suspension could be used. In some embodiments, sonic nebulizers are used because they minimize exposing the agent to shear, which can result in degradation of the compound.

Ordinarily, an aqueous aerosol can be made by formulating an aqueous solution or suspension of the agent together with conventional pharmaceutically acceptable carriers and stabilizers. The carriers and stabilizers vary with the requirements of the particular composition, but typically include nonionic surfactants (TWEEN® (polysorbates), PLURONIC® (poloxamers), sorbitan esters, lecithin, CREMOPHOR® (polyethoxylates)), pharmaceutically acceptable co-solvents such as polyethylene glycol, innocuous proteins like serum albumin, sorbitan esters, oleic acid, lecithin, amino acids such as glycine, buffers, salts, sugars, or sugar alcohols. Aerosols generally are prepared from isotonic solutions.

Transdermal patches have the added advantage of providing controlled delivery of a compound provided herein to the body. Such dosage forms can be made by dissolving or dispersing the agent in the proper medium. Absorption enhancers can also be used to increase the flux of the compound across the skin. The rate of such flux can be controlled by either providing a rate controlling membrane or dispersing the compound in a polymer matrix or gel.

The pharmaceutical compositions can also be prepared in the form of suppositories or retention enemas for rectal and/or vaginal delivery. Compositions presented as a suppository can be prepared by mixing one or more compounds provided herein with one or more suitable nonirritating excipients or carriers comprising, for example, cocoa butter, glycerides, polyethylene glycol, a suppository wax or a salicylate, which is solid at room temperature, but liquid at body temperature and, therefore, will melt in the rectum or vaginal cavity and release the active agent. Compositions which are suitable for vaginal administration also include pessaries, tampons, creams, gels, pastes, foams, or spray compositions containing such carriers as are known in the art to be appropriate.

A compound as disclosed herein can be prepared with carriers that will protect the compound against rapid elimination from the body, such as a controlled release composition, including implants and microencapsulated delivery systems. Biodegradable, biocompatible polymers can be used, such as ethylene vinyl acetate, polyanhydrides, polyglycolic acid, collagen, polyorthoesters, and polylactic acid. Such compositions can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811, which is incorporated herein by reference in its entirety.

As described above, the preparations of one or more compounds provided herein may be given orally, parenterally, topically, or rectally. They are, of course, given by forms suitable for each administration route. For example, they are administered in tablets or capsule form, by injection, inhalation, eye lotion, ointment, suppository, infusion; topically by lotion or ointment; and rectally by suppositories. In some embodiments, administration is oral.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion.

The phrases “systemic administration”, “administered systemically”, “peripheral administration”, and “administered peripherally” as used herein mean the administration of a ligand, drug, or other material via route other than directly into the central nervous system, such that it enters the patient's system and thus, is subject to metabolism and other like processes, for example, subcutaneous administration.

A compound provided herein may be administered to humans and other animals for therapy by any suitable route of administration, including orally, nasally, as by, for example, a spray, rectally, intravaginally, parenterally, intracistemally, and topically, as by powders, ointments or drops, including buccally and sublingually. Regardless of the route of administration selected, a compound provided herein, which may be used in a suitable hydrated form, and/or the pharmaceutical compositions provided herein, is formulated into a pharmaceutically acceptable dosage form by conventional methods known to those of skill in the art. In another embodiment, the pharmaceutical composition is an oral solution or a parenteral solution. Another embodiment is a freeze-dried preparation that can be reconstituted prior to administration. As a solid, this composition may also include tablets, capsules or powders.

Actual dosage levels of the active ingredients in the pharmaceutical compositions provided herein may be varied so as to obtain “therapeutically effective amount,” which is an amount of the active ingredient effective to achieve the desired therapeutic response for a particular patient, composition, and mode of administration, without being toxic to the patient.

The concentration of a compound provided herein in a pharmaceutically acceptable mixture will vary depending on several factors, including the dosage of the compound to be administered, the pharmacokinetic characteristics of the compound(s) employed, and the route of administration. In some embodiments, the compositions provided herein can be provided in an aqueous solution containing about 0.1-10% w/v of a compound disclosed herein, among other substances, for parenteral administration. Typical dose ranges can include from about 0.01 to about 50 mg/kg of body weight per day, given in 1-4 divided doses. Each divided dose may contain the same or different compounds. The dosage will be a therapeutically effective amount depending on several factors including the overall health of a patient, and the composition and route of administration of the selected compound(s).

Dosage forms or compositions containing a compound as described herein in the range of 0.005% to 100% with the balance made up from non-toxic carrier may be prepared. Methods for preparation of these compositions are known to those skilled in the art. The contemplated compositions may contain 0.001%-100% active ingredient, in one embodiment 0.1-95%, in another embodiment 75-85%. Although the dosage will vary depending on the symptoms, age and body weight of the patient, the nature and severity of the disorder to be treated or prevented, the route of administration and the form of the drug, in general, a daily dosage of from 0.01 to 2000 mg of the compound is recommended for an adult human patient, and this may be administered in a single dose or in divided doses. The amount of active ingredient which can be combined with a carrier material to produce a single dosage form will generally be that amount of the compound which produces a therapeutic effect.

The pharmaceutical composition may be administered at once, or may be divided into a number of smaller doses to be administered at intervals of time. It is also noted that the dose of the compound can be varied over time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the embodimented compositions.

The precise time of administration and/or amount of the composition that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the patient and adjusting the dosage and/or timing.

The pharmaceutical compositions can be included in a container, pack, or dispenser together with instructions for administration.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

It is to be understood that while the disclosure is read in conjunction with the detailed description thereof, the foregoing description is intended to illustrate and not limit the scope of the disclosure, which is defined by the scope of the appended claims. Other aspects, advantages, and modifications are within the scope of the following claims.

### Examples

The following examples are provided for illustration and are not intended to limit the scope of the disclosure in any way.

As used throughout these examples, common organic abbreviations are defined as follows:

| Abbreviation | Chemical |
|---|---|
| Ac | Acetyl |
| Ac₂O | Acetic anhydride |
| B₂pin₂ | Bis(pinacolato)diboron |
| BINAP | 2,2′-bis(diphenylphosphino)-1,1′- |
| | binaphthyl |
| Bn | Benzyl |
| BOC or Boc | tert-Butoxycarbonyl |
| BTFFH | Bis(tetramethylene)fluoroforma- |
| | midinium hexafluorophosphate |
| Bu | Butyl |
| BrettPhos Pd G3 | [(2-Di-cyclohexylphosphino-3,6- |
| | dimethoxy-2′,4′,6′- triisopropyl-1,1′- |
| | biphenyl)-2-(2′-amino-1,1′ - |
| | biphenyl)]palladium(II) |
| | methanesulfonate |
| Bz | Benzoyl |
| CMBP | (Tributylphosphoranylidene)ace- |
| | tonitrile |
| DABCO | 1,4-diazabicyclo[2.2.2]octane |
| DAST | (diethylamino)sulfur trifluoride |
| DBAD | Di-tertbutyl azodicarboxylate |
| DCM | Methylene chloride |
| DIBAL | Diisobutylammonium hydride |
| DIAD | Diisopropyl azodicarboxylate |
| DIEA/DIPEA | Diisopropylethylamine |
| DMAP | 4-(dimethylamino)pyridine |
| DMF | N,N′-Dimethylformamide |
| DMSO | Dimethylsulfoxide |
| dtpf (e.g., | 1,1′-bis(di-tert- |
| Pd(dtpf)Cl₂) | butylphosphino)ferrocene |
| EDCI | 1-Ethyl-3-(3- |
| | dimethylaminopropyl)carbodiimide |
| EtOAc | Ethyl acetate |
| HATU | 1-[Bis(dimethylamino)methylene]- |
| | 1H-1,2,3-triazolo[4,5-b]pyridinium 3- |
| | oxide hexafluorophosphate |
| KOtBu | Potassium tert-butoxide |
| LDA | Lithium diisopropylamide |
| mCBPA | meta-Chloroperoxybenzoic acid |
| MsCl | Mesyl chloride |
| NBS | N-bromosuccinimide |
| NMI | 1-methylimidazole |
| NMP | Methylpyrrolidone |
| Pd/C | Palladium on activated carbon |
| PHB | pyrrolidinone hydrotribromide |
| [Ph₃PBn]⁺Cl⁻ | benzyltriphenylphosphonium |
| | chloride |
| PPh₃ | Triphenylphopshine |
| TBAF | Tetrabutylammonium fluoride |
| TCFH | N,N,N′,N′- |
| | tetramethylchloroformamidinium |
| | hexafluorophosphate |
| TEA or NEt₃ | Triethylamine |
| TFA | Trifluoroacetic acid |
| THF | Tetrahydrofuran |
| TMS | Trimethylsilyl |
| TMSOK | potassium trimethylsiolate |
| XantPhOS or | 4,5-Bis(diphenylphosphino)-9,9- |
| XantPhos | dimethylxanthene |
| XPhOS | 2-Dicyclohexylphosphino-2′,4′,6′- |
| | triisopropylbiphenyl |
| XPhOS Pd G3 | (2-Dicyclohexylphosphino-2′,4′,6′- |
| | triisopropyl-1,1′-biphenyl)[2-(2′- |
| | amino-1,1′-biphenyl)]palladium(II) |
| | methanesulfonate |

### Synthetic Examples

Amine Synthesis:

### Route 1:

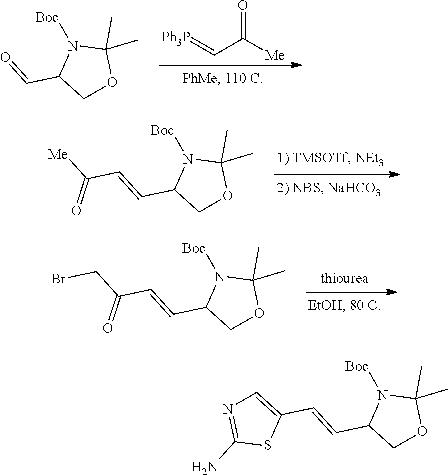

A 100 mL vial with stir bar was charged with tert-butyl 4-formyl-2,2-dimethyl-1,3-oxazolidine-3-carboxylate (1.00 g, 4.36 mmol, 1.00 equiv), 1-(triphenyl-lambda5-phosphanylidene)propan-2-one (3.02 mg, 9.60 mmol, 2.20 equiv) and toluene (20.00 mL) under nitrogen atmosphere. The vial was capped and placed in a 110° C. bath. The reaction mixture was stirred at 110° C. overnight. The next morning, the reaction mixture was cooled to room temperature and concentrated under vacuum. The resulting crude material was purified via silica gel chromatography to yield the desired product.

A 100 mL vial with stir bar was charged with tert-butyl 2,2-dimethyl-4-[(1E)-3-oxobut-1-en-1-yl]-1,3-oxazolidine-3-carboxylate (800.00 mg, 2.97 mmol, 1.00 equiv), TEA (450.83 mg, 4.46 mmol, 1.50 equiv), and toluene (10.00 mL) under nitrogen atmosphere, TMSOTf (858.20 mg, 3.86 mmol, 1.30 equiv) in toluene (2 mL) was added. The vial was capped and placed in a 0° C. bath. The reaction mixture was stirred at 0° C. for 2 h. The reaction mixture was then quenched by NaHCO₃(aq) (20 mL). The resulting solution was extracted with DCM (3×30 mL) and washed with brine (2×30 mL), and the organic layers were dried over Na₂SO₄, filtered and concentrated in vacuo. The resulting crude material was used directly for next step.

A 100 mL vial with stir bar was charged with tert-butyl 2,2-dimethyl-4-[(1E)-3-[(trimethylsilyl)oxy]buta-1,3-dien-1-yl]-1,3-oxazolidine-3-carboxylate (1.00 g, 2.93 mmol, 1.00 equiv), NaHCO₃ (368.96 mg, 4.39 mmol, 1.50 equiv) and THF (10.00 mL) under nitrogen atmosphere, NBS (573.26 mg, 3.22 mmol, 1.10 equiv) was added. The vial was capped and placed in a 0° C. bath. The reaction mixture was stirred at 0° C. for 30 min. The resulting mixture was then quenched by NaHCO₃(aq) (10 mL), the resulting solution was extracted with DCM (3×40 mL) and washed with brine (2×40 mL), and the organic layers were dried over Na₂SO₄, filtered and concentrated in vacuo. The resulting crude material was used directly for next step.

A 100 mL vial with stir bar was charged with tert-butyl 4-[(1E)-4-bromo-3-oxobut-1-en-1-yl]-2,2-dimethyl-1,3-oxazolidine-3-carboxylate (1.00 g, 2.87 mmol, 1.00 equiv), thiourea (437.17 mg, 5.74 mmol, 2.00 equiv) and EtOH (20.00 mL) under nitrogen atmosphere. The vial was capped and placed in a 70° C. bath. The reaction mixture was stirred at 70° C. for 2 h. The reaction mixture was cooled to room temperature and concentrated under vacuum. The reaction mixture was then quenched by NaHCO₃(aq) (20 mL). The resulting solution was extracted with DCM (3×40 mL). The combined organic layers were dried over Na₂SO₄, filtered and concentrated in vacuo. The resulting crude material was purified via silica gel chromatography to yield the desired product.

The following compounds were prepared via a similar method:

| | Compound name |
|---|---|
| B14 and | tert-butyl (E)-4-(2-(2-aminothiazol-5-yl)vinyl)-2,2- |
| B12 | dimethyloxazolidine-3-carboxylate |
| B16 | tert-butyl (E)-4-(2-(2-aminothiazol-4-yl)vinyl)-2,2,4- |
| | trimethyloxazolidine-3-carboxylate |
| B8 and | tert-butyl (E)-2-(2-(2-aminothiazol-4-yl)vinyl)-3,4- |
| B7 | dihydroquinoline-1(2H)-carboxylate |
| B9, B10, | tert-butyl (E)-2-(2-(2-aminothiazol-4-yl)vinyl)morpholine- |
| and B6 | 4-carboxylate |
| B18 | (E)-4-(2-(4-phenylmorpholin-2-yl)vinyl)thiazol-2-amine |
| E15 | tert-butyl (E)-(3-(2-aminothiazol-4-yl)allyl)(methyl)car- |
| | bamate |
| E30 and | tert-butyl (E)-(3-(2-aminothiazol-4-yl)allyl)carbamate |
| E54 | |
| A11, E16, | tert-butyl (E)-2-(2-(2-aminothiazol-4-yl)vinyl)piperidine-1- |
| and E18 | carboxylate |

### Route 2:

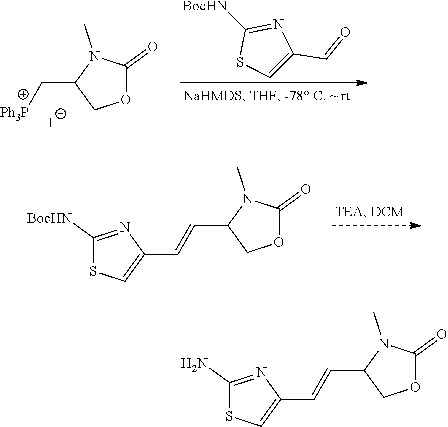

A 50 mL vial with stir bar was charged with [(3-methyl-2-oxo-1,3-oxazolidin-4-yl)methyl]triphenylphosphanium iodide (200.00 mg, 0.40 mmol, 1.00 equiv) and THF (10.00 mL) under nitrogen atmosphere. The vial was capped and placed in a −78° C. bath, NaHMDS (0.40 mL, 2.00 mol/L, 2.00 equiv) was added at at −78° C., the resulting solution was stirred for 20 min at −78° C. Tert-butyl N-(4-formyl-1,3-thiazol-2-yl)carbamate (348.00 mg, 0.40 mmol, 1.00 equiv) in THF (1 mL) at −78° C. was added. The resulting solution was stirred for 12 h at room temperature. The reaction was then quenched by NH₄Cl (aq) (50 mL). The resulting solution was extracted with EtOAc (3×50 mL) and washed with brine (1×50 mL). The combined organic layers were dried over Na₂SO₄, filtered and concentrated in vacuao. The resulting crude material was purified via silica gel chromatography to yield the desired product.

A 50 mL vial with stir bar was charged with tert-butyl N-[4-[(E)-2-(3-methyl-2-oxo-1,3-oxazolidin-4-yl)ethenyl]-1,3-thiazol-2-yl]carbamate (180.00 mg, 0.55 mmol, 1.00 equiv) and DCM (2.00 mL), TEA (2.00 ml) was added. The vial was capped and placed in a room temperature bath. The reaction mixture was stirred at room temperature for h. The resulting solution was concentrated in vacuo. The pH value of the solution was adjusted to 8 with NaHCO₃ (aq). The resulting solution was extracted with EtOAc (3×30 ml) and washed with brine (1×20 mL). The combined organic layers were dried over Na₂SO₄, filtered and concentrated in vacuo. The resulting crude material was purified via silica gel chromatography & RP column to yield the desired product.

The following compounds were prepared via a similar method:

| | Compound name | Phosphine used |
|---|---|---|
| B15 | (E)-4-(2-(2-aminothiazol-4-yl)vinyl)-3-methyloxazolidin- | PPh3, NaHMDS |
| | 2-one | |
| B2 and | (E)-4-(2-(2-methyloxazol-4-yl)vinyl)thiazol-2-amine | PPh3, NaHMDS |
| E4 | | |
| B5 | (E)-4-(2-(isoxazol-3-yl)vinyl)thiazol-2-amine | PPh3, NaHMDS |
| B1 | (E)-4-(2-(oxazol-4-yl)vinyl)thiazol-2-amine | PPh3, NaHMDS |
| E12 | 4-(2-(2-phenyloxazol-4-yl)ethyl)thiazol-2-amine | PPh3, NaHMDS |
| B33 | (E)-4-(2-(5-methyloxazol-4-yl)vinyl)thiazol-2-amine | PPh3, NaHMDS |
| and | | |
| B47 | | |
| B34 | (E)-4-(2-(oxazol-2-yl)vinyl)thiazol-2-amine | P(nBu)3, KOtBu |
| B35 | (E)-4-(2-(5-methyloxazol-2-yl)vinyl)thiazol-2-amine | P(nBu)3, KOtBu |
| B36 | (E)-4-(2-(2-(tert-butyl)oxazol-4-yl)vinyl)thiazol-2-amine | PPh3, NaHMDS |
| B38 | (E)-4-(2-(2-isopropyloxazol-4-yl)vinyl)thiazol-2-amine | PPh3, NaHMDS |
| B39 | (E)-4-(2-(2-cyclohexyloxazol-4-yl)vinyl)thiazol-2-amine | PPh3, NaHMDS |
| B40 | (E)-4-(2-(2-cyclopropyloxazol-4-yl)vinyl)thiazol-2-amine | PPh3, NaHMDS |
| B41 | (E)-4-(2-(4-methyloxazol-2-yl)vinyl)thiazol-2-amine | P(nBu)3, KOtBu |
| B52, | (E)-4-(2-(5-cyclohexyloxazol-4-yl)vinyl)thiazol-2-amine | PPh3, NaHMDS |
| B53, | | |
| and D4 | | |
| B54, | (E)-4-(2-(1-cyclohexyl-1H-imidazol-4-yl)vinyl)thiazol-2- | PPh3, NaHMDS |
| B55, | amine | |
| and D5 | | |
| B73 | (E)-4-(2-(5-isopropylimidazo[1,2-a]pyridin-2- | PPh3, NaHMDS |
| and | yl)vinyl)thiazol-2-amine | |
| B76 | | |
| B78 | (E)-4-(2-(5-ethylimidazo[1,2-a]pyridin-2-yl)vinyl)thiazol- | PPh3, NaHMDS |
| and | 2-amine | |
| B79 | | |
| B88 | (E)-4-(2-(5-methylisoxazol-3-yl)vinyl)thiazol-2-amine | PPh3, NaHMDS |
| B89 | (E)-4-(2-(5-ethylisoxazol-3-yl)vinyl)thiazol-2-amine | PPh3, NaHMDS |
| B90 | (E)-4-(2-(5-isopropylisoxazol-3-yl)vinyl)thiazol-2-amine | PPh3, NaHMDS |
| B92 | (E)-4-(2-(1-(tetrahydro-2H-pyran-4-yl)-1H-imidazol-4- | PPh3, NaHMDS |
| | yl)vinyl)thiazol-2-amine | |
| B125 | (E)-4-(2-(5-isopropyloxazol-4-yl)vinyl)thiazol-2-amine | PPh3, NaHMDS |
| B126 | (E)-4-(2-(5-ethyloxazol-4-yl)vinyl)thiazol-2-amine | PPh3, NaHMDS |
| B130 | (E)-4-(2-(5-(methoxymethyl)oxazol-4-yl)vinyl)thiazol-2- | PPh3, NaHMDS |
| | amine | |
| B131 | (E)-4-(2-(5-(tert-butyl)oxazol-4-yl)vinyl)thiazol-2-amine | PPh3, NaHMDS |
| B132 | (E)-4-(2-(5-cyclopropyloxazol-4-yl)vinyl)thiazol-2-amine | PPh3, NaHMDS |
| B133 | (E)-4-(2-(5-cyclobutyloxazol-4-yl)vinyl)thiazol-2-amine | PPh3, NaHMDS |

### Route 3:

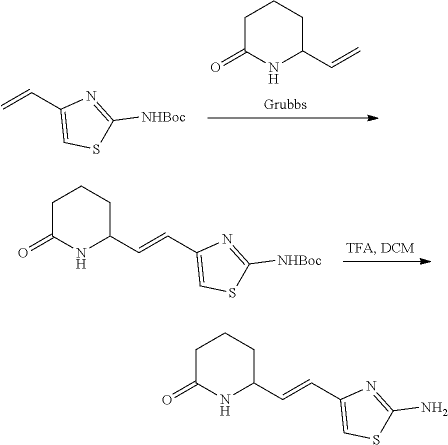

A 50 mL vial with stir bar was charged with tert-butyl N-(4-ethenyl-1,3-thiazol-2-yl)carbamate (100.00 mg, 0.44 mmol, 1.00 equiv), 6-ethenylpiperidin-2-one (27.66 mg, 0.22 mmol, 0.5 equiv), Grubbs 2nd (27.66 mg, 0.04 mmol, 0.10 equiv) and DCM (5.00 mL) under nitrogen atmosphere. The vial was capped and placed in a 40° C. bath. The reaction mixture was stirred at 40° C. overnight. The next morning, the resulting mixture was concentrated under vacuum. The resulting crude material was purified via RP column to yield the desired product.

The Boc group was removed as described in route 2.

| | Compound name |
|---|---|
| B13 | (E)-6-(2-(2-aminothiazol-4-yl)vinyl)piperidin-2-one |

### Route 4:

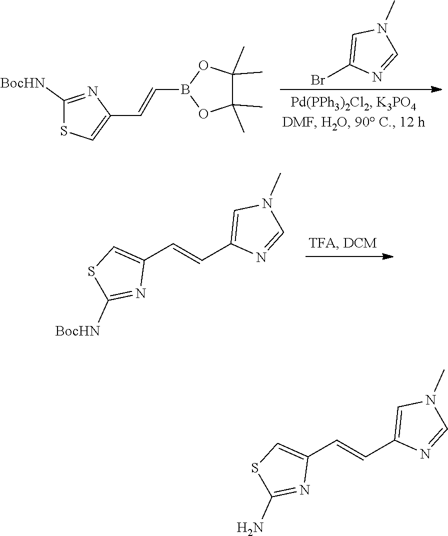

A 50 mL vial with stir bar was charged with tert-butyl N-(4-ethenyl-1,3-thiazol-2-yl)carbamate (100.00 mg, 0.44 mmol, 1.00 equiv), 6-ethenylpiperidin-2-one (27.66 mg, 0.22 mmol, 0.5 equiv), Grubbs 2nd (27.66 mg, 0.04 mmol, 0.10 equiv) and DCM (5.00 mL) under nitrogen atmosphere. The vial was capped and placed in a 40° C. bath. The reaction mixture was stirred at 40° C. overnight. The next morning, the resulting mixture was concentrated under vacuum. The resulting crude material was purified via RP column to yield the desired product.

The Boc group was removed as described in route 2.

The following compounds were prepared via a similar method:

| | Compound name |
|---|---|
| B3 | (E)-4-(2-(1-methyl-1H-imidazol-4-yl)vinyl)thiazol-2-amine |
| E57 | (E)-4-(2-(5-isopropylpyridin-2-yl)vinyl)thiazol-2-amine |
| E56 | (E)-4-(2-(3-isopropylpyridin-2-yl)vinyl)thiazol-2-amine |
| E55 | (E)-4-(2-(6-isopropylpyridin-2-yl)vinyl)thiazol-2-amine |
| B20 | (E)-4-(2-(1-methyl-1H-imidazol-2-yl)vinyl)thiazol-2-amine |
| D2 | (E)-4-(2-(3,5-difluoropyridin-2-yl)vinyl)thiazol-2-amine |
| B43 | (E)-4-(2-(pyrazin-2-yl)vinyl)thiazol-2-amine |
| B 44 | (E)-4-(2-(pyrimidin-4-yl)vinyl)thiazol-2-amine |
| B37, B45, B63, | (E)-4-(2-(1-isopropyl-1H-imidazol-4-yl)vinyl)thiazol-2-amine |
| B68, D7, B99, | |
| and D8 | |
| B48, B49, and | (E)-4-(2-(6-fluoro-5-methylpyridin-2-yl)vinyl)thiazol-2-amine |
| D3 | |
| B50 | (E)-4-(2-(imidazo[1,2-a]pyridin-2-yl)vinyl)thiazol-2-amine |
| B51 | (E)-4-(2-(1,2-dimethyl-1H-imidazol-4-yl)vinyl)thiazol-2-amine |
| B56 and C114 | (E)-4-(2-(1-(2-methoxyethyl)-1H-imidazol-4-yl)vinyl)thiazol-2-amine |
| B57 | (E)-4-(2-(1-isopropyl-1H-imidazol-5-yl)vinyl)thiazol-2-amine |
| B58 and B116 | (E)-4-(2-(1-isopropyl-5-methyl-1H-imidazol-4-yl)vinyl)thiazol-2-amine |
| B59 | (E)-4-(2-(6-methylpyridin-2-yl)vinyl)thiazol-2-amine |
| B60 | (E)-4-(2-(5,6,7,8-tetrahydroquinolin-2-yl)vinyl)thiazol-2-amine |
| B61 | (E)-4-(2-(6-methoxypyridin-2-yl)vinyl)thiazol-2-amine |
| B62 and B71 | (E)-4-(2-(6,7-dihydro-5H-pyrrolo[1,2-a]imidazol-2-yl)vinyl)thiazol-2-amine |
| B64, B66, B87, | (E)-4-(2-(5-isopropylpyridin-2-yl)vinyl)thiazol-2-amine |
| and D6 | |
| B65 and B69 | (E)-4-(2-(1-ethyl-1H-imidazol-4-yl)vinyl)thiazol-2-amine |
| B67 and B72 | (E)-4-(2-(2-isopropyl-1-methyl-1H-imidazol-4-yl)vinyl)thiazol-2-amine |
| B70 and B77 | (E)-4-(2-(5-methylimidazo[1,2-a]pyridin-2-yl)vinyl)thiazol-2-amine |
| B74 and B75 | (E)-4-(2-(1-isopropyl-1H-pyrazol-3-yl)vinyl)thiazol-2-amine |
| B80, B83, and | (E)-4-(2-(1-isopropyl-4-methyl-1H-pyrazol-3-yl)vinyl)thiazol-2-amine |
| B84 | |
| B81 and B85 | |
| B86 | (E)-4-(2-(5,6,7,8-tetrahydroimidazo[1,5-a]pyridin-1-yl)vinyl)thiazol-2-amine |
| B91 | (E)-1-(4-(4-(2-(2-aminothiazol-4-yl)vinyl)-1H-imidazol-1-yl)piperidin-1- |
| | yl)ethan-1-one |
| B93 | (E)-4-(2-(2-aminothiazol-4-yl)vinyl)-1-isopropyl-1H-imidazole-2-carbonitrile |
| B95 | (E)-4-(2-(1-isopropyl-1H-imidazol-2-yl)vinyl)thiazol-2-amine |
| B94 | (E)-4-(2-(3-isopropyl-1-methyl-1H-pyrazol-5-yl)vinyl)thiazol-2-amine |
| B96 | (E)-4-(2-(5-isopropyl-1-methyl-1H-pyrazol-3-yl)vinyl)thiazol-2-amine |
| B97 | (E)-4-(2-(7-methyl-5,6,7,8-tetrahydroimidazo[1,5-a]pyridin-1-yl)vinyl)thiazol- |
| | 2-amine |
| B98 | (E)-4-(2-(6-methyl-5,6,7,8-tetrahydroimidazo[1,5-a]pyridin-1-yl)vinyl)thiazol- |
| | 2-amine |
| B100 | (E)-4-(2-(5,6,7,8-tetrahydroimidazo[1,5-a]pyridin-3-yl)vinyl)thiazol-2-amine |
| B101 | (E)-4-(2-(5-methyl-5,6,7,8-tetrahydroimidazo[1,5-a]pyridin-1-yl)vinyl)thiazol- |
| | 2-amine |
| B102 | (E)-4-(2-(8-methyl-5,6,7,8-tetrahydroimidazo[1,5-a]pyridin-1-yl)vinyl)thiazol- |
| | 2-amine |
| B103 | ethyl (E)-4-(2-(2-aminothiazol-4-yl)vinyl)-1-isopropyl-1H-imidazole-2- |
| | carboxylate |
| B104 | (E)-4-(2-(1-phenyl-1H-imidazol-4-yl)vinyl)thiazol-2-amine |
| B105 | (E)-4-(2-(1-benzyl-1H-imidazol-4-yl)vinyl)thiazol-2-amine |
| B107 and B113 | (E)-2-(4-(2-(2-aminothiazol-4-yl)vinyl)-1H-imidazol-1-yl)propanenitrile |
| B108 and B109 | (E)-3-(4-(2-(2-aminothiazol-4-yl)vinyl)-1H-imidazol-1-yl)propanenitrile |
| B110 and B111 | (E)-3-(4-(2-(2-aminothiazol-4-yl)vinyl)-1H-imidazol-1-yl)butanenitrile |
| B112 and B115 | (E)-2-(4-(2-(2-aminothiazol-4-yl)vinyl)-1H-imidazol-1-yl)acetonitrile |
| B114 | (E)-4-(2-(1-((2-(trimethylsilyl)ethoxy)methyl)-1H-imidazol-4-yl)vinyl)thiazol-2- |
| | amine |
| B117 | (E)-4-(2-(1-ethyl-5-methyl-1H-imidazol-4-yl)vinyl)thiazol-2-amine |
| B118 and B119 | ethyl (E)-2-(4-(2-(2-aminothiazol-4-yl)vinyl)-5-methyl-1H-imidazol-1- |
| | yl)propanoate |
| B120 and B121 | methyl (E)-2-(4-(2-(2-aminothiazol-4-yl)vinyl)-5-methyl-1H-imidazol-1- |
| | yl)acetate |
| B122 | (E)-4-(2-(1-(2,2,2-trifluoroethyl)-1H-imidazol-4-yl)vinyl)thiazol-2-amine |
| B123 | (E)-4-(2-(1-(oxetan-3-yl)-1H-imidazol-4-yl)vinyl)thiazol-2-amine |
| B124 | (E)-4-(2-(1-(tetrahydrofuran-3-yl)-1H-imidazol-4-yl)vinyl)thiazol-2-amine |
| B127 | (E)-4-(2-(1-cyclobutyl-1H-imidazol-4-yl)vinyl)thiazol-2-amine |
| B128 | (E)-4-(2-(1-cyclopropyl-1H-imidazol-4-yl)vinyl)thiazol-2-amine |
| B129 | (E)-4-(2-(5-chloro-1-isopropyl-1H-imidazol-4-yl)vinyl)thiazol-2-amine |
| B134 | (E)-4-(2-(1-(tert-butyl)-1H-imidazol-4-yl)vinyl)thiazol-2-amine |

### Route 5:

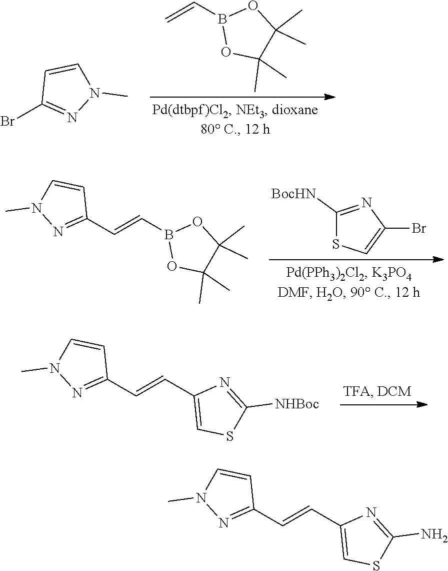

A 250 mL vial with stir bar was charged with 3-bromo-1-methylpyrazole (2.00 g, 12.42 mmol, 1.00 equiv), 2-ethenyl-4,4,5,5-tetramethyl-1,3,2-dioxaborolane (9.69 g, 62.11 mmol, 5.00 equiv), NEt₃ (6.35 g, 62.11 mmol, 5.00 equiv), Pd(dtbpf)Cl₂ (820.00 mg, 1.24 mmol, 0.10 equiv) and dioxane (80.00 mL) under nitrogen atmosphere. The vial was capped and placed in a 100° C. bath, the reaction mixture was stirred at 100° C. for 12 h. The resulting mixture was cooled to room temperature and concentrated under vacuum. The resulting crude material was purified via silica gel chromatography to yield the desired product.

A 100 mL vial with stir bar was charged with 1-methyl-3-[(E)-2-(4,4,5,5-tetramethyl-1,3,2-dioxaborolan-2-yl)ethenyl]pyrazole (670.00 mg, 2.86 mmol, 1.50 equiv), tert-butyl N-(4-bromo-1,3-thiazol-2-yl)carbamate (530.00 mg, 1.90 mmol, 1.00 equiv), K₃PO₄ (1.21 g, 5.72 mmol, 3.00 equiv), Pd(PPh₃)₂Cl₂ (268.00 mg, 0.38 mmol, 0.20 equiv), DMF (30 mL) and H₂O (6.00 mL) under nitrogen atmosphere. The vial was capped and placed in a 90° C. bath, the reaction mixture was stirred at 90° C. overnight. The resulting mixture was cooled to room temperature, poured into EtOAc (150 mL) and washed with brine (4×70 mL). The combined organic layers were dried over Na₂SO₄, filtered and concentrated in vacuo. The resulting crude material was purified via silica gel chromatography to yield the desired product.

The Boc group was removed as described in route 2.

The following compounds were prepared via similar method:

| | Compound name |
|---|---|
| B4 | (E)-4-(2-(1-methyl-1H-pyrazol-3-yl)vinyl)thiazol-2-amine |
| E1 | (E)-4-(2-(4-methylpyridin-2-yl)vinyl)thiazol-2-amine |
| E8 | 4-(2-(pyridin-2-yl)ethyl)thiazol-2-amine |
| E7 | 4-(2-(5-(trifluoromethoxy)pyridin-2-yl)ethyl)thiazol-2-amine |
| D1 | (E)-4-(2-(5-methylpyridin-2-yl)vinyl)thiazol-2-amine |

### Route 6:

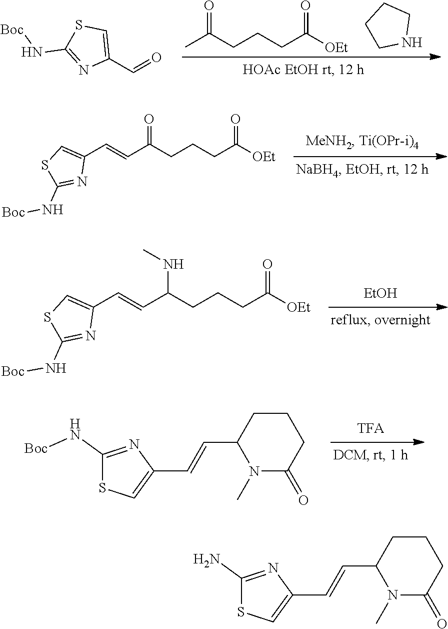

A 50 mL vial with stir bar was charged with tert-butyl N-(4-formyl-1,3-thiazol-2-yl)carbamate (300.00 mg, 1.31 mmol, 1.00 equiv), acetic acid (23.68 mg, 0.39 mmol, 0.30 equiv), pyrrolidine (28.04 mg, 0.39 mmol, 0.30 equiv), ethyl 5-oxohexanoate (249.49 mg, 1.58 mmol, 1.20 equiv) and EtOH (10.00 mL). The vial was capped and placed in a 25° C. bath. The reaction mixture was stirred at 25° C. overnight. The next morning, the reaction mixture was concentrated under vacuum. The reaction was then quenched by H₂O (20 mL). The resulting solution was extracted with EtOAc (3×20 mL) and washed with brine (1×20 mL). The combined organic layers were dried over Na₂SO₄, filtered and concentrated in vacuo. The resulting crude material was purified via silica gel chromatography to yield the desired product.

A 50 mL vial with stir bar was charged with ethyl (6E)-7-[2-[(tert-butoxycarbonyl)amino]-1,3-thiazol-4-yl]-5-oxohept-6-enoate (435.00 mg, 1.18 mmol, 1.00 equiv), Ti(OEt)₄ (671.90 mg, 2.95 mmol, 2.49 equiv), CH₃NH₂ (3.00 mL, 6.00 mmol, 5.08 equiv, 2M) and EtOH (3.00 mL) under nitrogen atmosphere. The vial was capped and placed in a 25° C. bath. The reaction mixture was stirred at 25° C. overnight. The next morning, NaBH₄ (89.80 mg, 2.37 mmol, 2.01 equiv) was added in portions at room temperature. The resulting solution was stirred at room temperature for 1 hr. The reaction was quenched by water (15 mL). The resulting solution was extracted with (3×20 mL) of ethyl acetate and washed with (1×20 mL) of brine. The organic layer was then dried over Na₂SO₄, filtered and concentrated in vacuo. The resulting crude material was used directly for next step.

A 50 mL vial with stir bar was charged with ethyl (6E)-7-[2-[(tert-butoxycarbonyl)amino]-1,3-thiazol-4-yl]-5-(methylamino)hept-6-enoate (400.00 mg, 1.04 mmol, 1.00 equiv) and ethyl alcohol (10.00 mL). The vial was capped and placed in a 70° C. bath. The reaction mixture was stirred at 70° C. for 2 h. The resulting mixture was concentrated under vacuum. The resulting crude material was purified via silica gel chromatography to yield the desired product.

The Boc group was removed as described in route 2.

| | Compound name |
|---|---|
| B19 | (E)-6-(2-(2-aminothiazol-4-yl)vinyl)-1-methylpiperidin-2-one |

### Route 7:

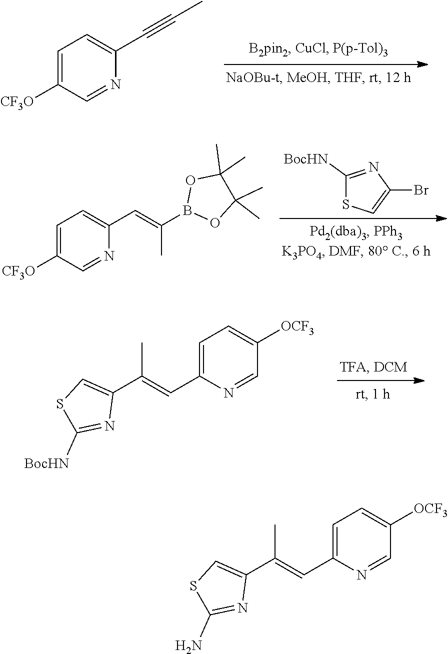

A 100 mL vial with stir bar was charged with a solution of t-BuONa (57.33 mg, 0.60 mmol, 0.40 equiv) CuCl (29.53 mg, 0.30 mmol, 0.20 equiv), tri-p-tolylphosphine (181.58 mg, 0.60 mmol, 0.40 equiv) in THF (6.00 mL) under nitrogen atmosphere. The mixture was stirred about 30 min at room temperature. This was followed by the addition of a solution of bis(pinacolato)diboron (454.59 mg, 1.79 mmol, 1.2 equiv) in THF (2 mL) at room temperature. The mixture was stirred about 10 min at room temperature. To this was added a solution of 2-(prop-1-yn-1-yl)-5-(trifluoromethoxy)pyridine (300 mg, 1.49 mmol, 1.00 equiv) and MeOH (95.58 mg, 2.98 mmol, 2.00 equiv) in THF (2 mL) at room temperature. The resulting solution was stirred for 6 h at room temperature. The reaction was then quenched by water (20 mL). The resulting solution was extracted with ethyl acetate (3×40 mL) and washed with brine (2×40 mL). The combined organic layers were dried over Na₂SO₄, filtered and concentrated in vacuo. The resulting crude material was purified via silica gel chromatography to yield the desired product.

A 50 mL vial with stir bar was charged with 2-[(1Z)-2-(4,4,5,5-tetramethyl-1,3,2-dioxaborolan-2-yl)prop-1-en-1-yl]-5-(trifluoromethoxy)pyridine (100.00 mg, 0.30 mmol, 1.00 equiv), tert-butyl N-(4-bromo-1,3-thiazol-2-yl)carbamate (101.40 mg, 0.36 mmol, 1.20 equiv), K₃PO₄ (193.30 mg, 0.91 mmol, 3.00 equiv), PPh₃ (31.70 mg, 0.12 mmol, 0.40 equiv), Pd₂(dba)₃ (55.70 mg, 0.06 mmol, 0.20 equiv) and DMF (12.00 mL) under nitrogen atmosphere. The resulting solution was stirred for 6 h at 80° C. The reaction mixture was cooled to room temperature. The reaction was then quenched by water (60 mL). The resulting solution was extracted with ethyl acetate (3×50 mL) and washed with (3×50 mL) of brine. The organic layers were dried over Na₂SO₄, filtered and concentrated in vacuo. The resulting crude material was purified via prep-TLC to yield the desired product.

The Boc group was removed as described in route 2.

The following compounds were prepared via a similar method:

| | Compound name |
|---|---|
| E2 | (E)-4-(1-(5-(trifluoromethoxy)pyridin-2-yl)prop-1-en-2-yl)thiazol- |
| | 2-amine |
| E5 | (E)-4-(2-(5-(trifluoromethoxy)pyridin-2-yl)prop-1-en-1-yl)thiazol- |
| | 2-amine |

### Route 8:

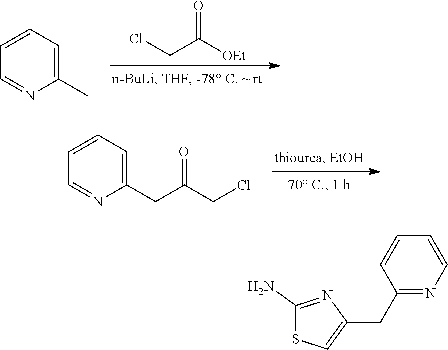

A 100 mL vial with stir bar was charged with 2-methylpyridine (1.00 g, 10.74 mmol, 1.00 equiv) and THF (20.00 mL) under nitrogen atmosphere, n-BuLi (5 ml, 2.5M, 1.20 equiv) was added at −78° C., the mixture solution was stirred 20 min at −78° C., and then ethyl chloroacetate (2.63 g, 21.48 mmol, 2.00 equiv) in THF (10 mL) was added at −78° C. The resulting solution was stirred for 2 hr at −78° C. The reaction was then quenched by NH₄Cl (aq) (100 mL). The resulting solution was extracted with DCM (3×100 mL) and washed with (2×100 mL) of brine. The organic layers were dried over Na₂SO₄, filtered and concentrated in vacuo. The resulting crude material was used directly for next step.

The condensation step was performed as described in route 1.

| | Compound name |
|---|---|
| E11 | 4-(pyridin-2-ylmethyl)thiazol-2-amine |

### Route 9:

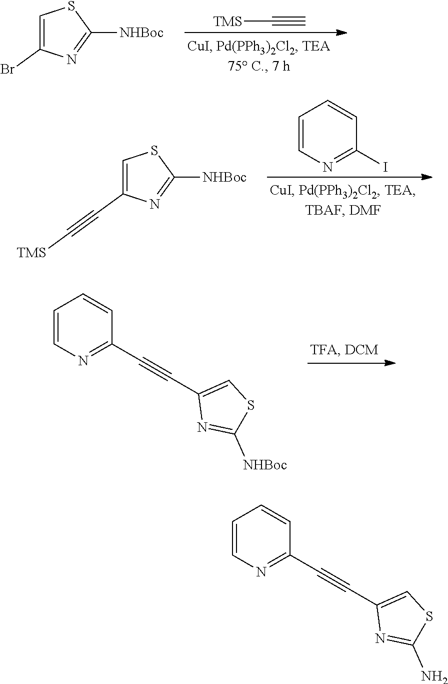

First Sonogashira (Step 1)

A 25 mL sealed tube with stir bar was charged with tert-butyl N-(4-bromo-1,3-thiazol-2-yl)carbamate (500.00 mg, 1.79 mmol, 1.00 equiv), TEA (6 mL), trimethylsilylacetylene (351.85 mg, 3.58 mmol, 2 equiv), CuI (17.06 mg, 0.09 mmol, 0.05 equiv), Pd(PPh₃)₂Cl₂ (188.58 mg, 0.27 mmol, 0.15 equiv) under nitrogen atmosphere. The resulting solution was stirred for 5 hr at 75° C. The reaction mixture was cooled to room temperature and concentrated under vacuum. The reaction was then quenched by H₂O (30 mL). The resulting solution was extracted with ethyl acetate (3×30 mL) and washed with (2×30 mL) of brine. The organic layers were dried over Na₂SO₄, filtered and concentrated in vacuo. The resulting crude material was purified via silica gel chromatography to yield the desired product.

Second Sonogashira (Step 2)

A 100 mL vial with stir bar was charged with tert-butyl N-[4-[2-(trimethylsilyl)ethynyl]-1,3-thiazol-2-yl]carbamate (300.00 mg, 1.01 mmol, 1.00 equiv), 2-iodopyridine (311.17 mg, 1.52 mmol, 1.50 equiv), CuI (19.27 mg, 0.10 mmol, 0.10 equiv), TEA (409.59 mg, 4.05 mmol, 4.00 equiv), Pd(PPh₃)₂Cl₂ (35.51 mg, 0.05 mmol, 0.05 equiv), TBAF (277.81 mg, 1.06 mmol, 1.05 equiv) and DMF (8 mL) under nitrogen atmosphere. The resulting solution was stirred for 12 hr at 80° C. in an oil bath. The reaction mixture was cooled to room temperature and concentrated under vacuum. The reaction was then quenched by H₂O (30 mL). The resulting solution was extracted with (3×30 mL) of ethyl acetate and washed with (1×30 mL) of brine. The organic layers were dried over Na₂SO₄, filtered and concentrated in vacuo. The resulting crude material was purified via silica gel chromatography to yield the desired product.

The Boc group was removed as described in route 2.

| | Compound name |
|---|---|
| E3 | 4-(pyridin-2-ylethynyl)thiazol-2-amine |

### Route 10:

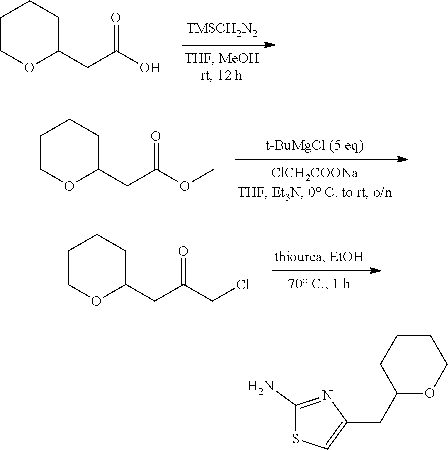

A 50 mL vial with stir bar was charged with oxan-2-ylacetic acid (300.00 mg, 2.08 mmol, 1.00 equiv), MeOH (1.00 mL) and THF (3.00 mL), TMSCHN₂ (2.1 mL, 2 M, 2.02 equiv) was added. The vial was capped and placed in a room temperature bath. The reaction mixture was stirred at room temperature overnight. The next morning, the resulting mixture was concentrated under vacuum. The resulting crude material was purified via silica gel chromatography to yield the desired product.

A 50 mL vial with stir bar was charged with methyl 2-(oxan-2-yl)acetate (283.00 mg, 1.79 mmol, 1.00 equiv), sodium 2-chloroacetate (623.30 mg, 5.35 mmol, 2.99 equiv), Et₃N (542.70 mg, 5.36 mmol, 3.00 equiv) and THF (8.00 mL), the contents were evacuated and backflushed with nitrogen. Tert-butyl(chloro)magnesium (7.0 mL, 1.7 M, 6.65 equiv) dropwise with stirring at 0° C. The vial was capped and placed in a room temperature bath. The reaction mixture was stirred at room temperature overnight. The next morning, the reaction was then quenched by citric acid(aq). The pH value of the solution was adjusted to 8 with NaHCO₃(aq). The resulting solution was extracted with DCM (3×20 mL) and washed with brine (1×20 mL). The organic layers were dried over Na₂SO₄, filtered and concentrated in vacuo. The resulting crude material was used directly for next step.

The condensation step was performed as described in route 1.

The following compounds were prepared via a similar method:

| | Compound name |
|---|---|
| E9 | 4-((tetrahydro-2H-pyran-2-yl)methyl)thiazol-2-amine |
| E37 | 4-(6,7-dihydro-5H-cyclopenta[b]pyridin-6-yl)thiazol-2-amine |

### Route 11:

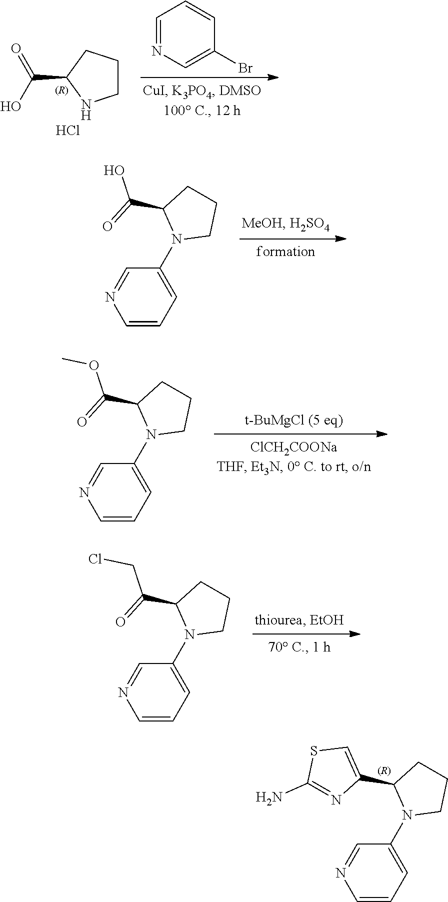

A 100 mL vial with stir bar was charged with 3-bromopyridine (500.00 mg, 3.17 mmol, 1.00 equiv), D-proline (910.00 mg, 7.91 mmol, 2.50 equiv), CuI (120.54 mg, 0.63 mmol, 0.20 equiv), K₃PO₄ (2.69 g, 12.66 mmol, 4.00 equiv) and DMSO (25.00 mL). The contents were evacuated and backflushed with nitrogen. The vial was capped and placed in a 100° C. bath. The reaction mixture was stirred at 100° C. overnight. The next morning, the reaction mixture was cooled to room temperature and concentrated under vacuum. The resulting crude material was used directly for next step.

A 100 mL vial with stir bar was charged with (2R)-1-(pyridin-3-yl)pyrrolidine-2-carboxylic acid (150.00 mg, 0.78 mmol, 1.00 equiv) and MeOH (10.00 mL), H₂SO₄ (1.00 mL, 18.76 mmol, 24.04 equiv) was added. The vial was capped and placed in a 60° C. bath. The reaction mixture was stirred at 60° C. for 4 h. The reaction mixture was cooled to room temperature. The pH value of the solution was adjusted to 8 with NaHCO₃(aq). The resulting solution was extracted with ethyl acetate (2×50 mL) and washed with H₂O (1×50 mL), brine (1×50 mL). The organic layers were dried over Na₂SO₄, filtered and concentrated in vacuo. The resulting crude material was used directly for next step.

The chloroketone formation step was performed as described in route 10.

The condensation step was performed as described in route 1.

| | Compound name |
|---|---|
| E46 | (R)-4-(1-(pyridin-3-yl)pyrrolidin-2-yl)thiazol-2-amine |

### Route 12:

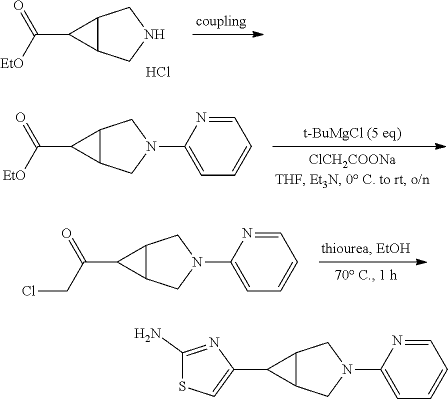 [SMILES: CCOC(=O)C1C2CNCC21 ; Cl ; CCOC(=O)C1C2CN(c3ccccn3)CC21 ; O=C(CCl)C1C2CN(c3ccccn3)CC21 ; Nc1nc(C2C3CN(c4ccccn4)CC32)cs1]

Coupling A: Buchwald Coupling

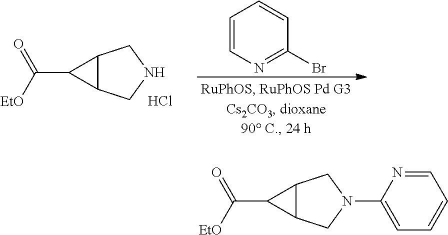 [SMILES: CCOC(=O)C1C2CNCC21 ; Cl ; Brc1ccccn1 ; CCOC(=O)C1C2CN(c3ccccn3)CC21]

A 100 mL vial with stir bar was charged with ethyl 3-azabicyclo[3.1.0]hexane-6-carboxylate hydrochloride (400.00 mg, 2.09 mmol, 1.00 equiv), 2-bromopyridine (494.62 mg, 3.13 mmol, 1.50 equiv), RuPhOS (194.78 mg, 0.42 mmol, 0.20 equiv), Cs₂CO₃ (2.04 g, 6.26 mmol, 3.00 equiv), RuPhos Palladacycle Gen.3 (349.11 mg, 0.42 mmol, 0.20 equiv) and dioxane (20.00 mL). The contents were evacuated and backflushed with nitrogen. The vial was capped and placed in a 80° C. bath. The reaction mixture was stirred at 80° C. overnight. The next morning, the reaction mixture was cooled to room temperature and poured into DCM (200 mL). The resulting mixture was washed with H₂O (1×50 mL) and brine (3×50 mL). The organic layers were dried over Na₂SO₄, filtered and concentrated in vacuo. The resulting crude material was purified via silica gel chromatography column to yield the desired product.

Coupling B: Chan-Lam Coupling

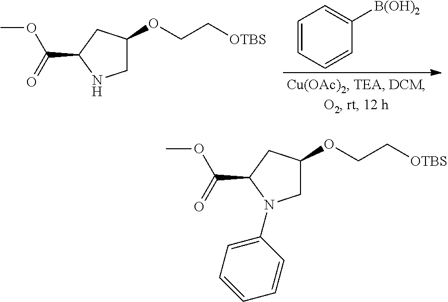 [SMILES: COC(=O)[C@H]1C[C@@H](OCCO[Si](C)(C)C(C)(C)C)CN1 ; OB(O)c1ccccc1 ; COC(=O)[C@H]1C[C@@H](OCCO[Si](C)(C)C(C)(C)C)CN1c1ccccc1]

A 100 mL vial with stir bar was charged with methyl (2R,4R)-4-[2-[(tert-butyldiphenylsilyl)oxy]ethoxy]pyrrolidine-2-carboxylate (100.00 mg, 0.23 mmol, 1.00 equiv), phenyl boronic acid (142.57 mg, 1.17 mmol, 5.00 equiv), TEA (59.16 mg, 0.59 mmol, 2.50 equiv), Cu(OAc)₂ (106.19 mg, 0.59 mmol, 2.50 equiv) and DCM (10.00 mL) under nitrogen atmosphere. The flask was then vacuumed and flushed with oxygen atmosphere, and the sequence was repeated twice. The vial was capped and placed in a room temperature bath. The reaction mixture was stirred at room temperature overnight under oxygen atmosphere using a oxygen balloon. The next morning, the reaction mixture was poured into DCM (50 mL) and quenched by the addition of NH₃·H₂O (5 mL), washed with H₂O (1×50 mL) and brine (3×50 mL). The organic layer was dried over Na₂SO₄, filtered and concentrated in vacuo. The resulting crude material was purified via silica gel chromatography to yield the desired product.

The chloroketone formation step was performed as described in route 10.

The condensation step was performed as described in route 1.

The following compounds were prepared via a similar method:

| | Coupling | |
|---|---|---|
| | protocol | Compound name |
| E33 | A | 4-(3-(pyridin-2-yl)-3-azabicyclo[3.1.0]hexan-6-yl)thiazol-2-amine |
| E36 | A | 4-(3-phenyl-3-azabicyclo[3.1.0]hexan-6-yl)thiazol-2-amine |
| E47 | A | (R)-4-(1-(pyridin-2-yl)piperidin-3-yl)thiazol-2-amine |
| E45 | A | (S)-4-(1-(pyridin-2-yl)piperidin-3-yl)thiazol-2-amine |
| E43 | A | (R)-4-(1-(pyridin-2-yl)pyrrolidin-3-yl)thiazol-2-amine |
| E44 | A | (S)-4-(1-(pyridin-2-yl)pyrrolidin-3-yl)thiazol-2-amine |
| E31 | B | 4-((2R,4R)-4-(2-((tert-butyldimethylsilyl)oxy)ethoxy)-1-phenylpyrrolidin-2- |
| and E32 | | yl)thiazol-2-amine |
| E40 | B | methyl (2R,4R)-4-((1-benzyl-1,2,3,6-tetrahydropyridin-4-yl)oxy)-1- |
| | | phenylpyrrolidine-2-carboxylate |
| A5 | B | (R)-4-(1-(4-isopropoxyphenyl)pyrrolidin-2-yl)thiazol-2-amine |
| A99 | A | tert-butyl (R)-(4-(2-(2-aminothiazol-4-yl)pyrrolidin-1-yl)-2,6- |
| | | difluorophenyl)carbamate |

### Route 13:

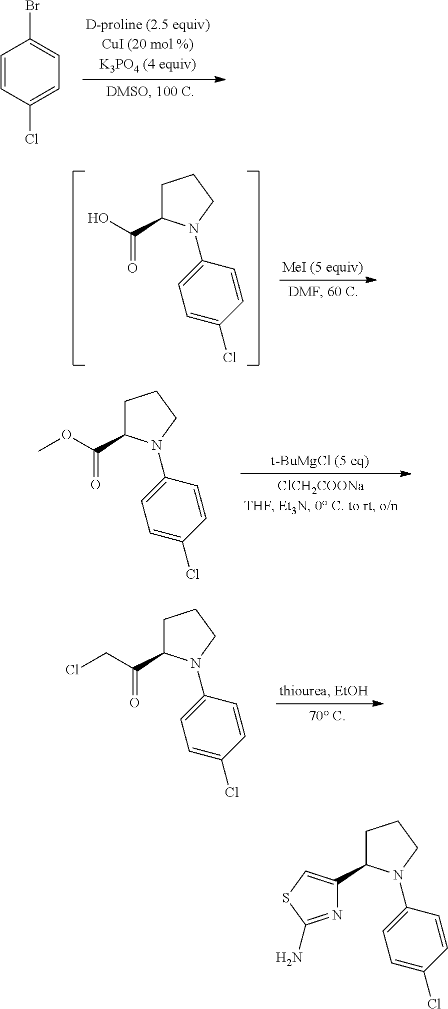

A 40 mL vial with stir bar was charged with D-proline (1.50 g, 13.1 mmol, 2.5 equiv), 1-bromo-4-chlorobenzene (1.00 g, 5.22 mmol, 1.0 equiv), CuI (199 mg, 1.04 mmol, 0.2 equiv) and K₃PO₄ (4.43 g, 20.9 mmol, 4.0 equiv). The contents were evacuated and backflushed with nitrogen. Degassed DMSO (7 mL) was added, and the vial was capped. The reaction mixture was stirred at 100 C overnight. The next morning, the reaction mixture was cooled to room temperature and diluted with DMF (10 mL). Iodomethane (1.63 mL, 26.1 mmol, 5.0 equiv) was added, and the reaction mixture was stirred at 60 C for 2 h. After 2 h, the reaction mixture was diluted with EtOAc (200 mL) and washed with brine (2×200 mL). The combined aqueous layers were extracted with EtOAc (1×100 mL). The combined organic layers were dried over Na₂SO₄, filtered and concentrated in vacuo. The resulting crude material was purified via silica gel chromatography to yield the desired product.

The chloroketone formation step was performed as described in route 10.

The condensation step was performed as described in route 1.

The following compounds were prepared via a similar method:

| | Compound name |
|---|---|
| A26 | (R)-4-(1-(4-chlorophenyl)pyrrolidin-2-yl)thiazol-2-amine |
| A9 | (R)-4-(1-(4-(oxazol-2-yl)phenyl)pyrrolidin-2-yl)thiazol-2-amine |
| A25 | (R)-4-(1-(4-fluorophenyl)pyrrolidin-2-yl)thiazol-2-amine |
| A22 | (R)-4-(1-(4-ethylphenyl)pyrrolidin-2-yl)thiazol-2-amine |
| A23 | (R)-4-(1-(4-isopropylphenyl)pyrrolidin-2-yl)thiazol-2-amine |
| A24 | (R)-4-(1-(4-cyclopropylphenyl)pyrrolidin-2-yl)thiazol-2-amine |
| A28 | (R)-4-(1-(4-(trifluoromethyl)phenyl)pyrrolidin-2-yl)thiazol-2-amine |
| A21 | (R)-4-(1-(p-tolyl)pyrrolidin-2-yl)thiazol-2-amine |
| E19 | (R)-4-(1-(6-methylpyridin-2-yl)pyrrolidin-2-yl)thiazol-2-amine |
| A31 | (R)-4-(1-(pyrimidin-5-yl)pyrrolidin-2-yl)thiazol-2-amine |
| Common | (R)-4-(1-phenylpyrrolidin-2-yl)thiazol-2-amine |
| intermediate | |
| E63 | (R)-4-(1-(4-methylpyridin-2-yl)pyrrolidin-2-yl)thiazol-2-amine |
| E62 | (R)-4-(1-(5-methylpyridin-2-yl)pyrrolidin-2-yl)thiazol-2-amine |
| A44, A45, A64, | tert-butyl (R)-(4-(2-(2-aminothiazol-4-yl)pyrrolidin-1-yl)benzyl)carbamate |
| and C104 | |
| C85 and E65 | (R)-4-(1-(5-ethylpyridin-2-yl)pyrrolidin-2-yl)thiazol-2-amine |
| A48 | (R)-4-(1-(4-(methoxymethyl)phenyl)pyrrolidin-2-yl)thiazol-2-amine |
| A57 | (R)-4-(1-(4-(((tert-butyldiphenylsilyl)oxy)methyl)phenyl)pyrrolidin-2-yl)thiazol-2-amine |
| A49 | tert-butyl (R)-(4-(2-(2-aminothiazol-4-yl)pyrrolidin-1-yl)benzyl)(methyl)carbamate |
| A51 and A55 | tert-butyl (R)-4-(4-(2-(2-aminothiazol-4-yl)pyrrolidin-1-yl)phenyl)piperidine-1- |
| | carboxylate |
| E64 | (R)-4-(1-(3-methylpyridin-2-yl)pyrrolidin-2-yl)thiazol-2-amine |
| C91 and A56 | (R)-4-(1-(4-(trifluoromethyl)phenyl)pyrrolidin-2-yl)thiazol-2-amine |
| C95 and C96 | (R)-4-(1-(6-ethylpyridin-3-yl)pyrrolidin-2-yl)thiazol-2-amine |
| A58 and A59 | tert-butyl 2-(4-((R)-2-(2-aminothiazol-4-yl)pyrrolidin-1-yl)phenyl)piperidine-1- |
| | carboxylate |
| A61 and A63 | tert-butyl 3-(4-((R)-2-(2-aminothiazol-4-yl)pyrrolidin-1-yl)phenyl)piperidine-1- |
| | carboxylate |
| common | tert-butyl (R)-4-(4-(2-(2-aminothiazol-4-yl)pyrrolidin-1-yl)phenoxy)piperidine-1- |
| intermediate | carboxylate |
| A65, and C105 | (R)-4-(2-(2-aminothiazol-4-yl)pyrrolidin-1-yl)benzonitrile |
| A66, A67, A70, | tert-butyl ((R)-1-(4-((R)-2-(2-aminothiazol-4-yl)pyrrolidin-1-yl)phenyl)ethyl)carbamate |
| and A72 | |
| common | tert-butyl (R)-(4-(2-(2-aminothiazol-4-yl)pyrrolidin-1-yl)phenyl)carbamate |
| intermediate | |
| A69, A71, A73, | tert-butyl ((S)-1-(4-((R)-2-(2-aminothiazol-4-yl)pyrrolidin-1-yl)phenyl)ethyl)carbamate |
| and A74 | |
| A75 | (R)-6-(2-(2-aminothiazol-4-yl)pyrrolidin-1-yl)-3,4-dihydroquinolin-2(1H)-one |
| A77 and C110 | (R)-4-(1-(4-ethylphenyl)pyrrolidin-2-yl)thiazol-2-amine |
| A78 | (R)-4-(1-(4-(2-((tert-butyldiphenylsilyl)oxy)ethyl)phenyl)pyrrolidin-2-yl)thiazol-2-amine |
| A93 | tert-butyl (R)-(4-(2-(2-aminothiazol-4-yl)pyrrolidin-1-yl)-2-fluorophenyl)carbamate |
| A95 | (R)-4-(1-(phenyl-3,5-d2)pyrrolidin-2-yl)thiazol-2-amine |
| A96 | tert-butyl (R)-(4-(2-(2-aminothiazol-4-yl)pyrrolidin-1-yl)-3,5-difluorophenyl)carbamate |
| A97 | tert-butyl (R)-4-(4-(2-(2-aminothiazol-4-yl)pyrrolidin-1-yl)phenoxy)-4-methylpiperidine- |
| | 1-carboxylate |
| A98 | tert-butyl (R)-(4-(2-(2-aminothiazol-4-yl)pyrrolidin-1-yl)-3-fluorophenyl)carbamate |
| A100 | tert-butyl (R)-4-(4-(2-(2-aminothiazol-4-yl)pyrrolidin-1-yl)-3-fluorophenoxy)piperidine-1- |
| | carboxylate |
| A101, A104, | tert-butyl (R)-(2-(4-(2-(2-aminothiazol-4-yl)pyrrolidin-1-yl)phenoxy)ethyl)carbamate |
| and A121 | |
| A102 | tert-butyl (R)-4-(4-(2-(2-aminothiazol-4-yl)pyrrolidin-1-yl)-2-fluorophenoxy)piperidine-1- |
| | carboxylate |
| A103 and A117 | tert-butyl (R)-(2-(4-(2-(2-aminothiazol-4-yl)pyrrolidin-1- |
| | yl)phenoxy)ethyl)(methyl)carbamate |
| A105 | tert-butyl (R)-4-(4-(2-(2-aminothiazol-4-yl)pyrrolidin-1-yl)-3,5- |
| | difluorophenoxy)piperidine-1-carboxylate |
| A106 | tert-butyl (R)-4-(4-(2-(2-aminothiazol-4-yl)pyrrolidin-1-yl)-2,6- |
| | difluorophenoxy)piperidine-1-carboxylate |
| A111 | 4-((R)-1-(4-((R)-1-((tert-butyldimethylsilyl)oxy)ethyl)phenyl)pyrrolidin-2-yl)thiazol-2- |
| | amine |
| A112 | 4-((R)-1-(4-((S)-1-((tert-butyldimethylsilyl)oxy)ethyl)phenyl)pyrrolidin-2-yl)thiazol-2- |
| | amine |
| A113 | (R)-4-(1-(4-(((tert-butyldimethylsilyl)oxy)methyl)phenyl)pyrrolidin-2-yl)thiazol-2-amine |
| A114 | (R)-4-(1-(4-(1-((tert-butyldimethylsilyl)oxy)cyclobutyl)phenyl)pyrrolidin-2-yl)thiazol-2- |
| | amine |
| A126 | tert-butyl (R)-4-(4-(2-(2-aminothiazol-4-yl)pyrrolidin-1-yl)-2-cyanophenoxy)piperidine- |
| | 1-carboxylate |
| A129 | tert-butyl (R)-4-(4-(2-(2-aminothiazol-4-yl)pyrrolidin-1-yl)-2- |
| | methoxyphenoxy)piperidine-1-carboxylate |
| A133 | tert-butyl (R)-4-(4-(2-(2-aminothiazol-4-yl)pyrrolidin-1-yl)-3-cyanophenoxy)piperidine- |
| | 1-carboxylate |
| A140 | tert-butyl (R)-4-(4-(2-(2-aminothiazol-4-yl)pyrrolidin-1-yl)-2-chlorophenoxy)piperidine- |
| | 1-carboxylate |
| A142 | (S)-4-(1-(4-(trifluoromethyl)phenyl)pyrrolidin-2-yl)thiazol-2-amine |
| A143 | tert-butyl (R)-4-(4-(2-(2-aminothiazol-4-yl)pyrrolidin-1-yl)-3-chlorophenoxy)piperidine- |
| | 1-carboxylate |
| A154 and A156 | tert-butyl (R)-4-((6-(2-(2-aminothiazol-4-yl)pyrrolidin-1-yl)pyridin-3-yl)oxy)piperidine-1- |
| | carboxylate |
| A155 and A159 | tert-butyl (R)-4-((5-(2-(2-aminothiazol-4-yl)pyrrolidin-1-yl)pyridin-2-yl)oxy)piperidine-1- |
| | carboxylate |
| A160 | tert-butyl (R)-4-(4-(2-(2-aminothiazol-4-yl)pyrrolidin-1-yl)benzyl)piperidine-1- |
| | carboxylate |
| A161 | tert-butyl 4-(1-(4-((R)-2-(2-aminothiazol-4-yl)pyrrolidin-1-yl)phenyl)ethyl)piperidine-1- |
| | carboxylate |
| A175 and A180 | tert-butyl (R)-(6-(2-(2-aminothiazol-4-yl)pyrrolidin-1-yl)pyridin-3-yl)carbamate |

### Route 14:

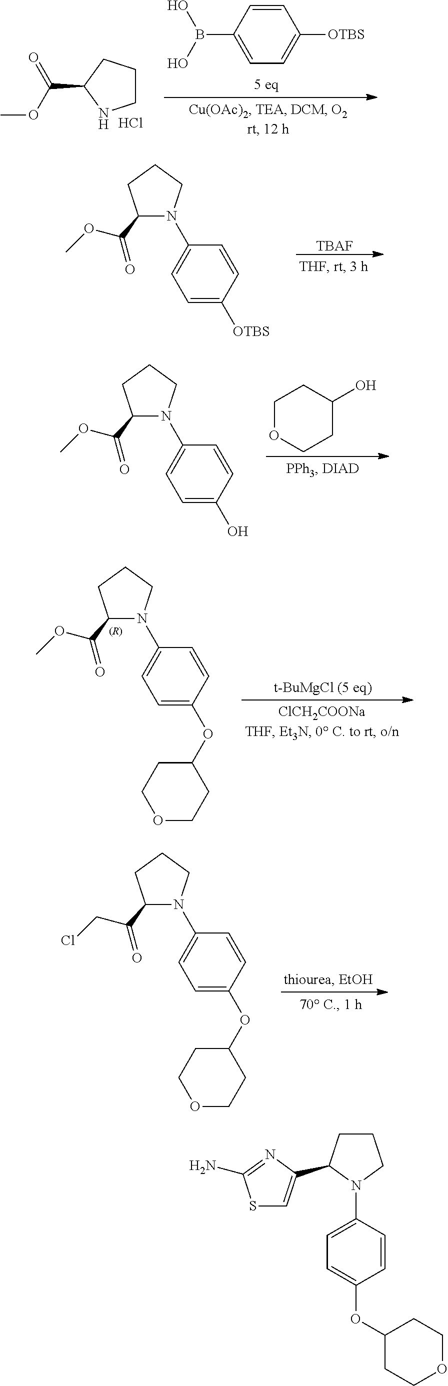

The Chan-Lam coupling step was performed as described in route 12.

A 250 mL vial with stir bar was charged with methyl (2R)-1-[4-[(tert-butyldimethylsilyl)oxy]phenyl]pyrrolidine-2-carboxylate (1.37 g, 4.08 mmol, 1.00 equiv) and THF (20.00 mL), TBAF (3.20 g, 12.24 mmol, 3.00 equiv) was added. The resulting solution was stirred at room temperature for 4 h. The reaction was then quenched by water (70 mL). The resulting solution was extracted with ethyl acetate (3×70 mL) and washed with brine (1×70 mL). The combined organic layers were dried over Na₂SO₄, filtered and concentrated in vacuo. The resulting crude material was purified via silica gel chromatography to yield the desired product.

Procedure A: Mitsonobu Coupling

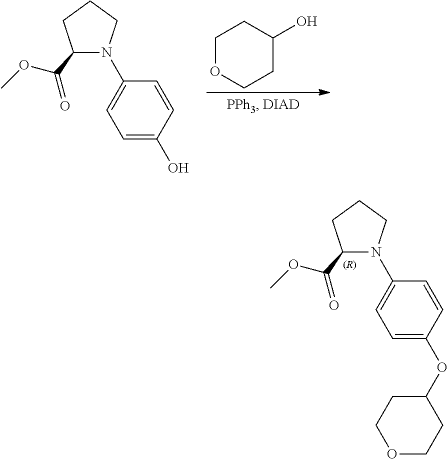

Into a 100-mL round-bottom flask, was placed methyl (2R)-1-(4-hydroxyphenyl)pyrrolidine-2-carboxylate (260.00 mg, 1.18 mmol, 1.00 equiv), oxan-4-ol (140.00 mg, 1.37 mmol, 1.20 equiv), PPh₃ (463.00 mg, 1.77 mmol, 1.50 equiv) and toluene (15 mL) under nitrogen atmosphere. A solution of DIAD (356.40 mg, 1.76 mmol, 1.50 equiv) in toluene (5 mL) dropwise with stirring at 0° C. The resulting solution was stirred at o n° C. overnight. The next morning, the reaction mixture was cooled to room temperature and quenched by water (50 mL). The resulting solution was extracted with ethyl acetate (3×50 ml) and washed with brine (1×50 mL). The combined organic layers were dried over Na₂SO₄, filtered and concentrated in vacuo. The resulting crude material was purified via silica gel chromatography to yield the desired product.

Procedure B: SN2 Coupling

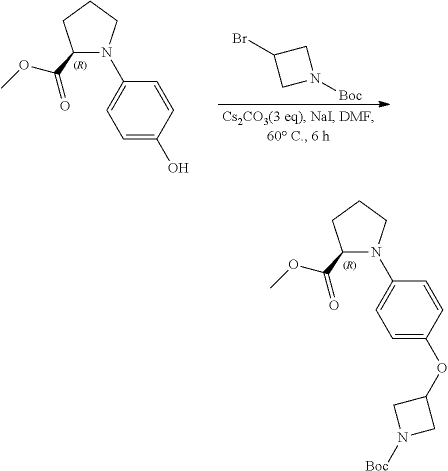

The chloroketone formation step was performed as described in route 10.

The condensation step was performed as described in route 1.

The following compounds were prepared via a similar method:

| | Coupling | |
|---|---|---|
| | protocol | Compound name |
| A1 | A | (R)-4-(1-(4-((tetrahydro-2H-pyran-4-yl)oxy)phenyl)pyrrolidin-2-yl)thiazol-2-amine |
| A7, A8, | A | tert-butyl (R)-4-(4-(2-(2-aminothiazol-4-yl)pyrrolidin-1-yl)phenoxy)piperidine-1- |
| and A6 | | carboxylate |
| A2 | A | (R)-4-(1-(4-(oxetan-3-yloxy)phenyl)pyrrolidin-2-yl)thiazol-2-amine |
| A4 | A | 4-((R)-1-(4-(((S)-tetrahydrofuran-3-yl)oxy)phenyl)pyrrolidin-2-yl)thiazol-2-amine |
| A3 | A | 4-((R)-1-(4-(((R)-tetrahydrofuran-3-yl)oxy)phenyl)pyrrolidin-2-yl)thiazol-2-amine |
| A79 | A | tert-butyl 3-(4-((R)-2-(2-aminothiazol-4-yl)pyrrolidin-1-yl)phenoxy)pyrrolidine-1- |
| and A80 | | carboxylate |
| A83 | A | tert-butyl (R)-3-(4-((R)-2-(2-aminothiazol-4-yl)pyrrolidin-1-yl)phenoxy)piperidine-1- |
| and A87 | | carboxylate |
| A84 | A | tert-butyl (S)-3-(4-((R)-2-(2-aminothiazol-4-yl)pyrrolidin-1-yl)phenoxy)piperidine-1- |
| and A88 | | carboxylate |
| A107 and | A | tert-butyl 4-(4-((R)-2-(2-aminothiazol-4-yl)pyrrolidin-1-yl)phenoxy)-3- |
| A108 | | fluoropiperidine-1-carboxylate |
| Common | B | tert-butyl (R)-3-(4-(2-(2-aminothiazol-4-yl)pyrrolidin-1-yl)phenoxy)azetidine-1- |
| intermediate | | carboxylate |
| A127 | A | (R)-4-(1-(4-(2-methoxyethoxy)phenyl)pyrrolidin-2-yl)thiazol-2-amine |
| A134 | A | (R)-4-(1-(4-(2-((tert-butyldiphenylsilyl)oxy)ethoxy)phenyl)pyrrolidin-2-yl)thiazol-2- |
| | | amine |
| A138 | A | (R)-4-(4-(2-(2-aminothiazol-4-yl)pyrrolidin-1-yl)phenoxy)tetrahydro-2H-thiopyran |
| | | 1,1-dioxide |
| A157 and | A | (R)-4-(1-(4-(thietan-3-yloxy)phenyl)pyrrolidin-2-yl)thiazol-2-amine |
| A158 | | |
| A170 | A | (R)-4-(1-(4-((tetrahydro-2H-thiopyran-4-yl)oxy)phenyl)pyrrolidin-2-yl)thiazol-2- |
| | | amine |

### Route 15:

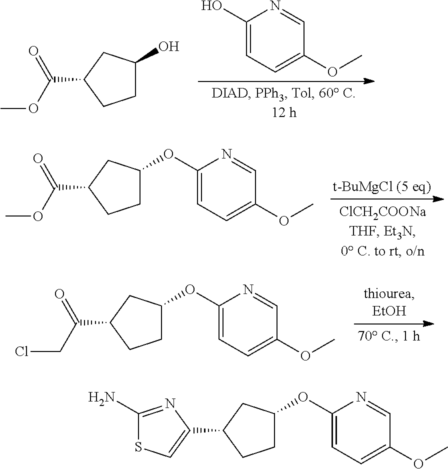

A 100 mL vial with stir bar was charged methyl (1S,3S)-3-hydroxycyclopentane-1-carboxylate (200.00 mg, 1.39 mmol, 1.00 equiv), 5-methoxypyridin-2-ol (208.30 mg, 1.67 mmol, 1.20 equiv), PPh₃ (545.78 mg, 2.08 mmol, 1.50 equiv) and toluene (15 mL) under nitrogen atmosphere. A solution of DIAD (420.77 mg, 2.08 mmol, 1.50 equiv) in toluene (5 mL) dropwise with stirring at 0° C. The vial was capped and placed in a 60° C. bath. The reaction mixture was stirred at 60° C. overnight. The reaction mixture was cooled to room temperature and concentrated under vacuum. The reaction was then quenched by H₂O (20 mL). The resulting solution was extracted with ethyl acetate (3×30 mL) and washed with (2×30 mL) of brine. The organic layers were dried over Na₂SO₄, filtered and concentrated in vacuo. The resulting crude material was purified via silica gel chromatography to yield the desired product.

The chloroketone formation step was performed as described in route 10.

The condensation step was performed as described in route 1.

The following compounds were prepared via a similar method:

| | Compound name |
|---|---|
| E42 | 4-((1S,3R)-3-((5-methoxypyridin-2-yl)oxy)cyclopentyl)thiazol-2-amine |
| E34 | 4-((1r,4r)-4-((5-methoxypyridin-2-yl)oxy)cyclohexyl)thiazol-2-amine |
| E38 | 4-((1S,3S)-3-((5-methoxypyridin-2-yl)oxy)cyclopentyl)thiazol-2-amine |
| E41 | 4-((1S,3S)-3-((5-methoxypyridin-2-yl)oxy)cyclohexyl)thiazol-2-amine |
| E39 | 4-((1S,3R)-3-((5-methoxypyridin-2-yl)oxy)cyclohexyl)thiazol-2-amine |
| E35 | 4-((1s,4s)-4-((5-methoxypyridin-2-yl)oxy)cyclohexyl)thiazol-2-amine |

### Route 16:

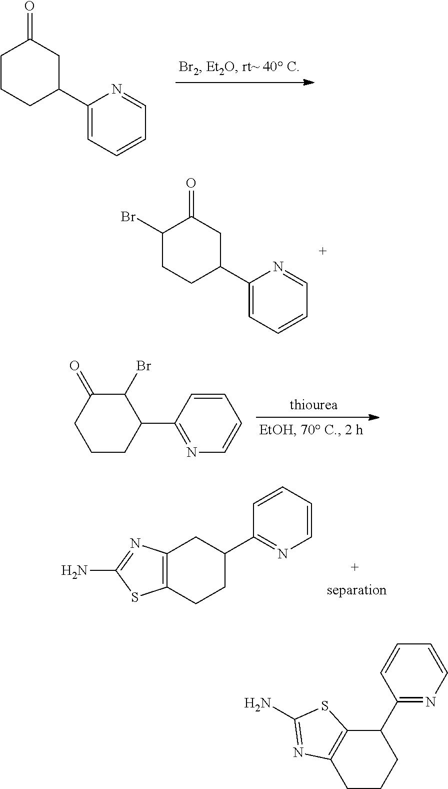 [SMILES: O=C1CCCC(c2ccccn2)C1 ; O=C1CC(c2ccccn2)CCC1Br ; O=C1CCCC(c2ccccn2)C1Br ; Nc1nc2c(s1)CCC(c1ccccn1)C2 ; Nc1nc2c(s1)C(c1ccccn1)CCC2]

A 50 mL vial with stir bar was charged with 3-(94thenone-2-yl)cyclohexan-1-one (200.00 mg, 1.14 mmol, 1.00 equiv) in Et₂O (5.00 mL, 0.04 M), Br₂ (181.00 mg, 1.13 mmol, 1.00 equiv) was added, the vial was capped and placed in an 25° C. bath. The reaction mixture was stirred at 25° C. for 1 h. The reaction was then quenched by H₂O (20 mL). The pH value of the solution was adjusted to 8 with sat.NaHCO₃(aq). The resulting solution was extracted with ethyl acetate (3×30 mL) and washed with brine (1×50 mL). The organic layer was then dried over Na₂SO₄, filtered and concentrated in vacuo. The resulting crude material was used directly for next step.

A 50 mL vial with stir bar was charged with 2-bromo-5-(94thenone-2-yl)cyclohexan-1-one (200.00 mg, 0.79 mmol, 1.00 equiv) in EtOH (10.00 mL, 0.079 M), the vial was capped and placed in an 70° C. bath. The reaction mixture was stirred at 70° C. for 2 h. The resulting mixture was concentrated under vacuum and quenched by H₂O (20 mL). The pH value of the solution was adjusted to 8 with sat.NaHCO₃(aq). The resulting solution was extracted with DCM (3×30 mL) and washed with brine (1×30 mL). The resulting crude material was purified via silica gel chromatography to yield the desired product.

### Route 17:

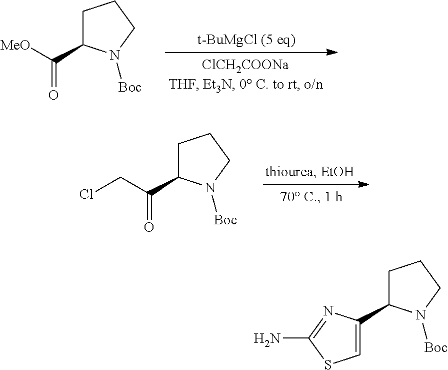 [SMILES: COC(=O)[C@H]1CCCN1C(=O)OC(C)(C)C ; CC(C)(C)OC(=O)N1CCC[C@@H]1C(=O)CCl ; CC(C)(C)OC(=O)N1CCC[C@@H]1c1csc(N)n1]

A 500 mL vial with stir bar was charged with 1-tert-butyl 2-methyl (2R)-pyrrolidine-1,2-dicarboxylate (6.00 g, 26.17 mmol, 1.00 equiv), sodium 2-chloroacetate (9.14 g, 78.51 mmol, 3.00 equiv), Et₃N (7.94 g, 78.51 mmol, 3.00 equiv) and THF (200.00 mL, 0.13 M), the contents were evacuated and backflushed with nitrogen. Tert-butyl(chloro)magnesium (76.97 mL, 1.7 M, 5.00 equiv) dropwise with stirring at 0° C. The vial was capped and placed in a room temperature bath. The reaction mixture was stirred at room temperature overnight. The next morning, the reaction was then quenched by citric acid(aq). The pH value of the solution was adjusted to 8 with NaHCO₃(aq). The resulting solution was extracted with DCM (4×100 mL), and the combined organic layers washed with brine (1×200 mL). The organic layer was dried over Na₂SO₄, filtered and concentrated in vacuo. The resulting crude material was used directly for next step.

A 250 mL vial with stir bar was charged with tert-butyl (2R)-2-(2-chloroacetyl)pyrrolidine-1-carboxylate (5.00 g, 20.18 mmol, 1.00 equiv), thiourea (2.30 g, 30.22 mmol, 1.50 equiv) and EtOH (60.00 mL, 0.34 M) under nitrogen atmosphere. The vial was capped and placed in a 70° C. bath. The reaction mixture was stirred at 70° C. for 1 h. The reaction mixture was cooled to room temperature and concentrated under vacuum. The reaction mixture was then quenched by NaHCO₃(aq). The resulting solution was extracted with DCM (3×50 mL). The combined organic layers were dried over Na₂SO₄, filtered and concentrated in vacuo. The resulting crude material was purified via silica gel chromatography to yield the desired product.

### Route 18:

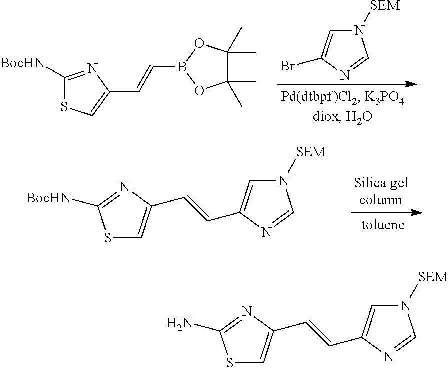

A 100 mL vial with stir bar was charged with tert-butyl N-{4-[€-2-(4,4,5,5-tetramethyl-1,3,2-dioxaborolan-2-yl)ethenyl]-1,3-thiazol-2-yl}carbamate (300.0 mg, 0.85 mmol, 1.00 equiv), 4-bromo-1-{[2-(trimethylsilyl)ethoxy]methyl}imidazole (283.3 mg, 1.02 mmol, 1.20 equiv), K₃PO₄ (560.4 mg, 2.64 mmol, 3.10 equiv), Pd(dtbpf)Cl₂ (111.0 mg, 0.17 mmol, 0.20 equiv) and dioxane (15.0 mL, 0.05 M) and H₂O (3.0 mL). The contents were evacuated and backflushed with nitrogen. The vial was capped and placed in an 80° C. bath. The reaction mixture was stirred at 80° C. for 2 h. The reaction mixture was cooled to room temperature. The reaction was then quenched by water. The resulting solution was extracted with ethyl acetate (3×20 mL), and the combined organic layers were washed with brine (1×60 mL). The organic layer was dried over Na₂SO₄, filtered and concentrated in vacuo. The resulting crude material was purified via silica gel chromatography to yield the desired product.

A 50 mL vial with stir bar was charged with tert-butyl N-{4-[€-2-(1-{[2-(trimethylsilyl)ethoxy]methyl}96thenone96-4-yl)ethenyl]-1,3-thiazol-2-yl}carbamate (300.0 mg, 0.71 mmol, 1.00 equiv), silica gel (3.00 g, 49.93 mmol, 70.34 equiv) and toluene (20.00 mL, 0.02 M). The contents were evacuated and backflushed with nitrogen. The vial was capped and placed in an 90° C. bath. The reaction mixture was stirred at 90° C. for 1 h. The resulting solution was concentrated in vacuo. The resulting crude material was purified via silica gel chromatography to yield the desired product.

### Route 19:

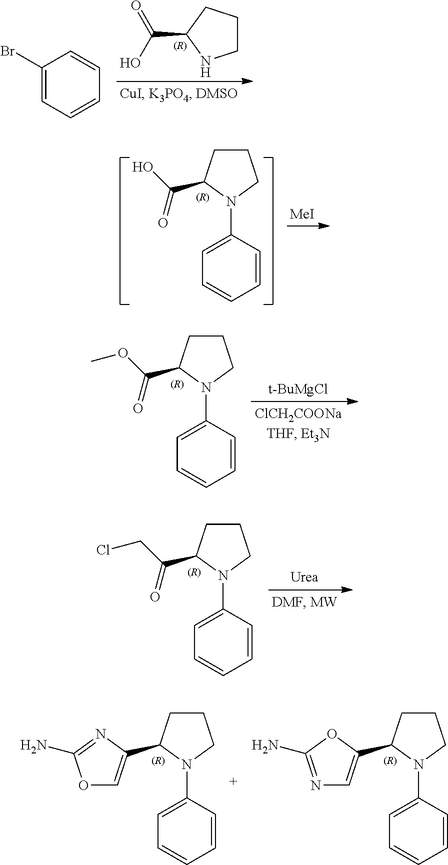

The Ullman coupling was performed as described in route 13.

The chloroketone formation step was performed as described in route 10.

A 30 mL sealed tube with stir bar was charged with 2-chloro-1-[(2R)-1-phenylpyrrolidin-2-yl]96thenone (500.00 mg, 2.24 mmol, 1.00 equiv), Urea (671.16 mg, 11.18 mmol, 5.00 equiv) and DMF (12.00 mL, 0.19 M) under nitrogen atmosphere. The sealed tube was capped and placed in a 120° C. microwave radiation. The reaction mixture was irradiated at 120° C. for 30 min. The reaction mixture was cooled to room temperature. The reaction mixture was then quenched by NaHCO₃(aq). The resulting solution was extracted with ethyl acetate (3×20 mL) and the combined organic layers were washed with brine (3×20 mL). The organic layer was dried over Na₂SO₄, filtered and concentrated in vacuo. The resulting crude material was purified via silica gel chromatography to yield the desired products.

### Route 20:

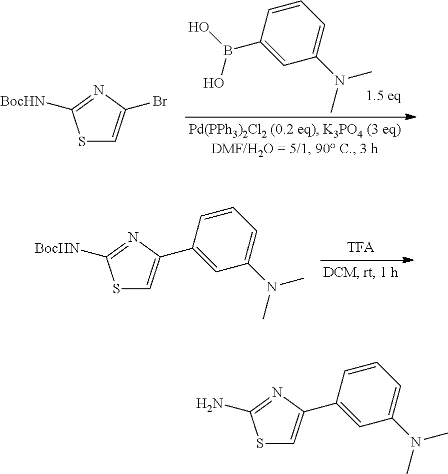 [SMILES: CC(C)(C)OC(=O)Nc1nc(Br)cs1 ; CN(C)c1cccc(B(O)O)c1 ; CN(C)c1cccc(-c2csc(NC(=O)OC(C)(C)C)n2)c1 ; CN(C)c1cccc(-c2csc(N)n2)c1]

A 100 mL vial with stir bar was charged with tert-butyl N-(4-bromo-1,3-thiazol-2-yl)carbamate (300.00 mg, 1.08 mmol, 1.00 equiv), 3-(dimethylamino)phenylboronic acid (265.99 mg, 1.61 mmol, 1.50 equiv), Pd(PPh₃)₂Cl₂ (150.87 mg, 0.22 mmol, 0.20 equiv), K₃PO₄ (684.36 mg, 3.22 mmol, 3.00 equiv), DMF (15 mL, 0.07 M) and H₂O (3 mL) was added under nitrogen atmosphere, and the vial was capped and placed in an 90° C. bath. The reaction mixture was stirred at 90° C. for 2 h. The reaction mixture was cooled to room temperature. The reaction mixture was poured into EA (200 mL) and washed with H₂O (1×100 mL), followed by brine (3×100 mL). The organic layer was then dried over Na₂SO₄, filtered and concentrated in vacuo. The resulting crude material was purified via silica gel chromatography to yield the desired product.

The Boc group was removed as described in route 2.

### Route 21:

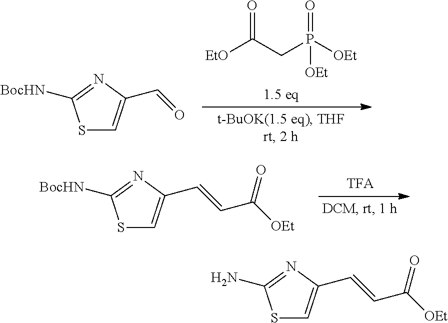

A 250 mL vial with stir bar was charged with t-BuOK (1.30 g, 11.57 mmol, 1.50 equiv) in dry THF (25 mL), triethyl phosphonoacetate (2.59 g, 11.57 mmol, 1.50 equiv) was added. The reaction mixture was stirred for 2 h at 25° C. under nitrogen atmosphere, tert-butyl N-(4-formyl-1,3-thiazol-2-yl)carbamate (1.76 g, 7.71 mmol, 1.00 equiv) in dry THF (40 mL, 0.12 M) was added dropwise over 10 min, and the vial was capped and placed in an 25° C. bath. The reaction mixture was stirred at 25° C. for 1 h. The reaction mixture was quenched with sat.NH₄Cl(aq) (100 mL). The mixture was extracted with EtOAc (3×100 mL) and the combined organic layers were washed with sat.NaHCO₃(aq) (1×100 mL) and brine (1×100 mL). The organic layer was then dried over Na₂SO₄, filtered and concentrated in vacuo. The resulting crude material was purified via silica gel chromatography to yield the desired product.

The Boc group was removed as described in route 2.

### Route 22:

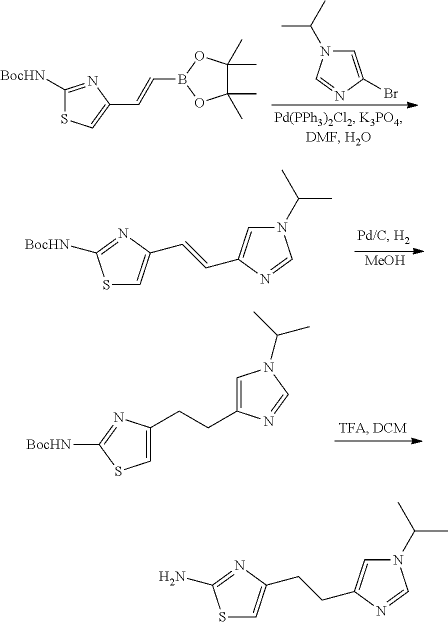

The Suzuki coupling was performed as described in route 4.

A 100 mL vial with stir bar was charged with tert-butyl N-{4-[€-2-(1-isopropylimidazol-4-yl)ethenyl]-1,3-thiazol-2-yl}carbamate (200 mg, 0.60 mmol, 1.00 equiv) and Pd/C (10%, 200 mg, 1.88 mmol, 3.13 equiv) in MeOH (10 mL, 0.06 M) under nitrogen atmosphere. The flask was then vacuumed and flushed with hydrogen. The reaction mixture was hydrogenated at room temperature for 1 hours under hydrogen atmosphere using a hydrogen balloon. Then the reaction mixture was filtered through a celite pad and the filtrate was concentrated under reduced pressure. The crude precipitated material was used in the next step without further purification.

The Boc group was removed as described in route 2.

### Route 23:

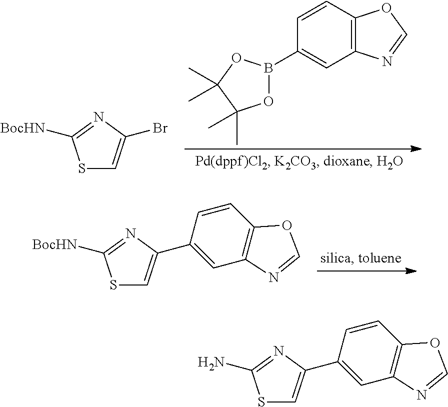

A 100 mL vial with stir bar was charged with 5-(4,4,5,5-tetramethyl-1,3,2-dioxaborolan-2-yl)-1,3-benzoxazole (500.00 mg, 2.04 mmol, 1.00 equiv), tert-butyl N-(4-bromo-1,3-thiazol-2-yl)carbamate (567.00 mg, 2.03 mmol, 1.00 equiv), Pd(dppf)Cl₂ (300.00 mg, 0.41 mmol, 0.20 equiv), K₂CO₃ (844.00 mg, 6.11 mmol, 3.00 equiv), dioxane (20 mL, 0.07 M) and H₂O (4 mL) was added under nitrogen atmosphere, and the vial was capped and placed in an 80° C. bath. The reaction mixture was stirred at 80° C. for 3 h. The reaction mixture was cooled to room temperature. The reaction mixture was poured into EA (300 mL) and washed with H₂O (1×100 mL), followed by brine (2×100 mL). The organic layer was then dried over Na₂SO₄, filtered and concentrated in vacuo. The resulting crude material was purified via silica gel chromatography to yield the desired product.

A 100 mL vial with stir bar was charged with 4-(1,3-benzoxazol-5-yl)-1,3-thiazol-2-amine (200.00 mg, 0.63 mmol, 1.00 equiv), silica gel (2.00 g, 33.23 mmol, 52.75 equiv) and toluene (15 mL, 0.04 M). The contents were evacuated and backflushed with nitrogen. The vial was capped and placed in an 90° C. bath. The reaction mixture was stirred at 90° C. for 1 h. The resulting solution was concentrated in vacuo. The resulting crude material was purified via silica gel chromatography to yield the desired product.

### Route 24:

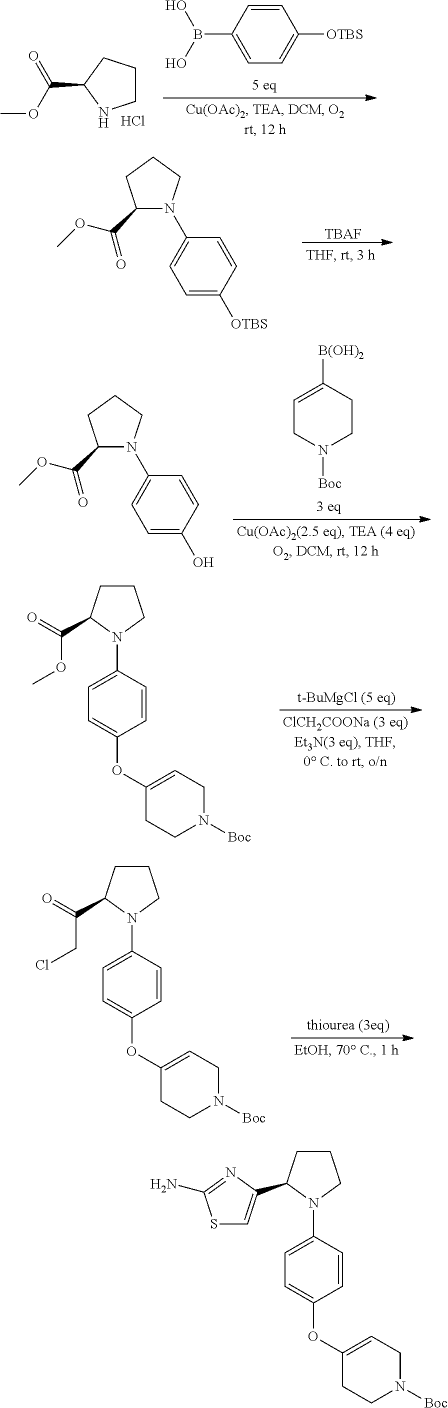

The Chan-Lam coupling with 4-(tert-butyldimethylsilyloxy)phenylboronic acid was performed as described in route 12.

The silyl deprotection was performed as described in route 14.

A 100 mL vial with stir bar was charged with methyl (2R)-1-(4-hydroxyphenyl)pyrrolidine-2-carboxylate (100 mg, 0.45 mmol, 1.00 equiv), 1-(tert-butoxycarbonyl)-3,6-dihydro-2H-pyridin-4-ylboronic acid (307.88 mg, 1.36 mmol, 3.00 equiv), Cu(OAc)₂ (245.38 mg, 1.36 mmol, 3.00 equiv), TEA (0.31 mL, 2.26 mmol, 5.00 equiv) and DCM (15.00 mL, 0.03 M) under nitrogen atmosphere. The flask was then vacuumed and flushed with oxygen. The reaction mixture was stirred at room temperature for 24 hours under oxygen atmosphere using an oxygen balloon. The reaction mixture was poured into DCM (50 mL) and quenched by the addition of NH₃·H₂O (5 mL), washed with H₂O (1×40 mL) and brine (3×40 mL). The organic layer was dried over Na₂SO₄, filtered and concentrated in vacuo. The resulting crude material was purified via silica gel chromatography to yield the desired product.

The chloroketone formation was performed as described in route 10.

The condensation step was performed as described in route 1.

### Route 25:

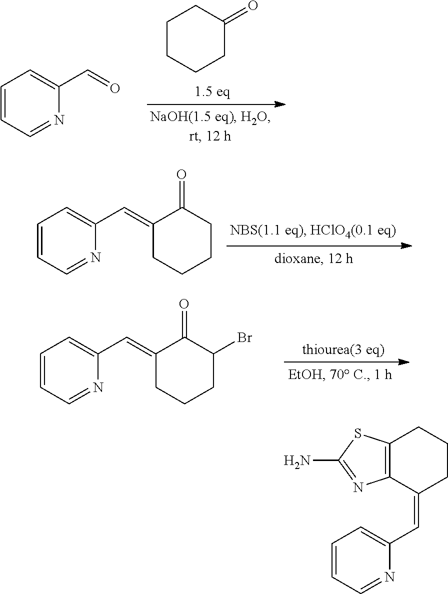

A 100 mL vial with stir bar was charged with 2-formylpyridine (5.00 g, 46.68 mmol, 1.00 equiv), cyclohexanone (6.87 g, 70.02 mmol, 1.50 equiv) and H₂O (30.00 mL, 0.20 M), NaOH (2.80 g, 70.02 mmol, 1.50 equiv) was added, and the vial was capped and placed in an 25° C. bath. The reaction mixture was stirred at 25° C. overnight. The next morning, the pH value of the reaction mixture was adjusted to 7 with HCl (aq) (1 M). The resulting mixture was concentrated in vacuo. The resulting crude material was purified via silica gel chromatography to yield the desired product.

A 50 mL vial with stir bar was charged with (2E)-2-(pyridin-2-ylmethylidene)cyclohexan-1-one (200.00 mg, 1.07 mmol, 1.00 equiv) in dioxane (10.00 mL, 0.11 M), NBS (209.12 mg, 1.18 mmol, 1.10 equiv), HClO₄ (21.46 mg, 0.21 mmol, 0.20 equiv) was added, and the vial was capped and placed in an 40° C. bath. The reaction mixture was stirred at 40° C. for 2 h. The reaction mixture was quenched by NaHCO₃(s). The solids were filtered out. The filtrate was concentrated in vacuo. The resulting crude material was used in the next step without further purification.

The condensation step was performed as described in route 1.

### Route 26:

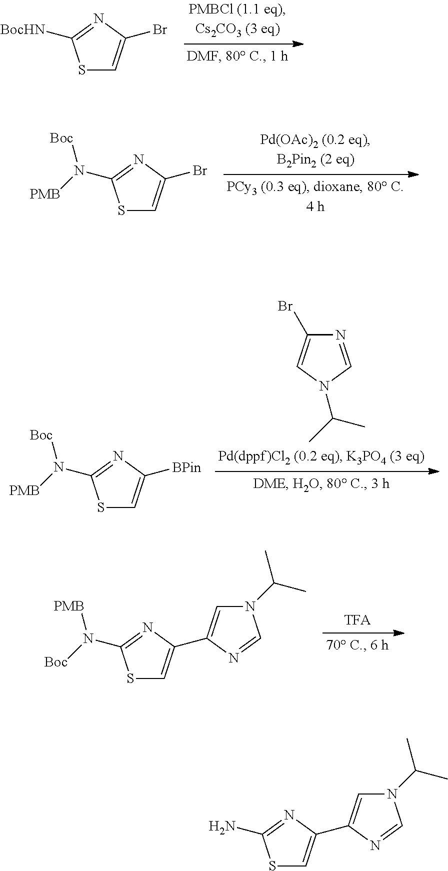

A 500 mL vial with stir bar was charged with tert-butyl N-(4-bromo-1,3-thiazol-2-yl)carbamate (6.00 g, 21.49 mmol, 1.00 equiv), Cs₂CO₃ (14.01 g, 42.99 mmol, 2.00 equiv) in DMF (150 mL, 0.14 M), PMBCl (4.04 g, 25.79 mmol, 1.20 equiv) was added. The vial was evacuated and backflushed with nitrogen. The vial was capped and placed in an 70° C. bath, and the reaction mixture was allowed to stir at 70° C. for 3 h. The reaction mixture was cooled to room temperature. The reaction mixture was poured into EtOAc (200 mL) and washed with brine (3×200 mL). The combined organic layers were dried over Na₂SO₄, filtered and concentrated in vacuo. The resulting crude material was purified via silica gel chromatography to yield the desired product.

A 100 mL vial with stir bar was charged with tert-butyl N-(4-bromo-1,3-thiazol-2-yl)-N-[(4-methoxyphenyl)methyl]carbamate (1.40 g, 3.51 mmol, 1.00 equiv), KOAc (860 mg, 8.77 mmol, 2.50 equiv), 4,4,5,5-tetramethyl-2-(tetramethyl-1,3,2-dioxaborolan-2-yl)-1,3,2-dioxaborolane (0.98 g, 3.88 mmol, 1.10 equiv), PCy₃ (290 mg, 1.05 mmol, 0.30 equiv), Pd(OAc)₂ (160 mg, 0.70 mmol, 0.20 equiv) and dioxane (25 mL, 0.14 M). The vial was evacuated and backflushed with nitrogen. The vial was capped and placed in an 80° C. bath, and the reaction mixture was allowed to stir at 80° C. for 3 h. The reaction mixture was cooled to room temperature. The reaction mixture was poured into EtOAc (150 mL) and washed with brine (2×100 mL). The combined organic layers were dried over Na₂SO₄, filtered and concentrated in vacuo. The resulting crude material was purified via silica gel chromatography to yield the desired product.

A 100 mL vial with stir bar was charged with tert-butyl N-[(4-methoxyphenyl)methyl]-N-[4-(4,4,5,5-tetramethyl-1,3,2-dioxaborolan-2-yl)-1,3-thiazol-2-yl]carbamate (700 mg, 1.57 mmol, 1.00 equiv), 4-bromo-1-isopropylimidazole (355.77 mg, 1.88 mmol, 1.2 equiv), K₃PO₄ (998.63 mg, 4.70 mmol, 3.00 equiv), Pd(dppf)Cl₂ (220.14 mg, 0.31 mmol, 0.20 equiv), DMF (15 mL, 0.09 M) and H₂O (3 mL). The vial was evacuated and backflushed with nitrogen. The vial was capped and placed in an 80° C. bath, and the reaction mixture was allowed to stir at 80° C. for 3 h. The reaction mixture was cooled to room temperature. The reaction mixture was poured into EtOAc (80 mL) and washed with brine (3×50 mL). The combined organic layers were dried over Na₂SO₄, filtered and concentrated in vacuo. The resulting crude material was purified via silica gel chromatography to yield the desired product.

A 50 mL vial with stir bar was charged with tert-butyl N-[4-(1-isopropylimidazol-4-yl)-1,3-thiazol-2-yl]-N-[(4-methoxyphenyl)methyl]carbamate (300 mg, 0.70 mmol, 1.00 equiv) and TFA (10 mL, 0.07 M). The vial was evacuated and backflushed with nitrogen. The vial was capped and placed in an 70° C. bath, and the reaction mixture was allowed to stir at 70° C. for 2 h. The reaction mixture was cooled to room temperature and concentrated in vacuo. The resulting mixture was dissolved in MeOH (20 mL). The pH value of the resulting solution was adjusted to 8 with with sat.NaHCO₃(aq). The reaction mixture was concentrated in vacuo. The resulting crude material was purified via RP column to yield the desired product.

The following compounds were prepared via a similar method:

| | Compound name |
|---|---|
| A162 | 4-(3-(pyridin-2-yl)phenyl)thiazol-2-amine |
| A167 | 4-(3-(1-isopropyl-1H-imidazol-4-yl)phenyl)thiazol-2-amine |
| A168 | 4-(2-(pyridin-2-yl)phenyl)thiazol-2-amine |
| A169 | 4-(2-(1-isopropyl-1H-imidazol-4-yl)phenyl)thiazol-2-amine |

### Route 27:

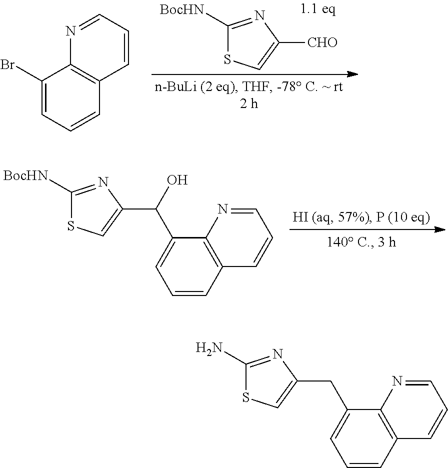 [SMILES: Brc1cccc2cccnc12 ; CC(C)(C)OC(=O)Nc1nc(C=O)cs1 ; CC(C)(C)OC(=O)Nc1nc(C(O)c2cccc3cccnc23)cs1 ; Nc1nc(Cc2cccc3cccnc23)cs1]

A 100 mL vial with stir bar was charged with 8-bromoquinoline (500 mg, 2.40 mmol, 1.00 equiv) in THF (20 mL). The flask was then vacuumed and flushed with nitrogen atmosphere. n-BuLi (1.44 mL, 3.60 mmol, 1.50 equiv) was added dropwise over 5 min at −78° C., the mixture was stirred for 30 min at −78° C. Tert-butyl N-(4-formyl-1,3-thiazol-2-yl)carbamate (603.43 mg, 2.64 mmol, 1.10 equiv) in dry THF (10 mL, 0.08 M) was added dropwise over 5 min at −78° C. And the vial was capped and placed in an −78° C. bath. The reaction mixture was stirred at −78° C. for 2 h. The reaction mixture was quenched with sat.NH₄Cl (aq) (60 mL). The mixture was extracted with EtOAc (3×80 mL) and the combined organic layers were washed with brine (2×70 mL). The organic layer was then dried over Na₂SO₄, filtered and concentrated in vacuo. The resulting crude material was purified via silica gel chromatography to yield the desired product.

A 100 mL vial with stir bar was charged with tert-butyl N-[4-[hydroxy(quinolin-8-yl)methyl]-1,3-thiazol-2-yl]carbamate (180.00 mg, 0.50 mmol, 1.00 equiv), P (155 mg, 5.00 mmol, 10 eq) and HI (5.00 mL, 57%, 0.10 M). And the vial was capped and placed in an 150° C. bath. The reaction mixture was stirred at 150° C. for 2 h. The reaction mixture was cooled to room temperature. The pH value of the resulting solution was adjusted to 8 with sat.NaHCO₃ (aq). The mixture was extracted with DCM (3×50 mL). The organic layer was then dried over Na₂SO₄, filtered and concentrated in vacuo. The resulting crude material was purified via silica gel chromatography to yield the desired product.

### Route 28:

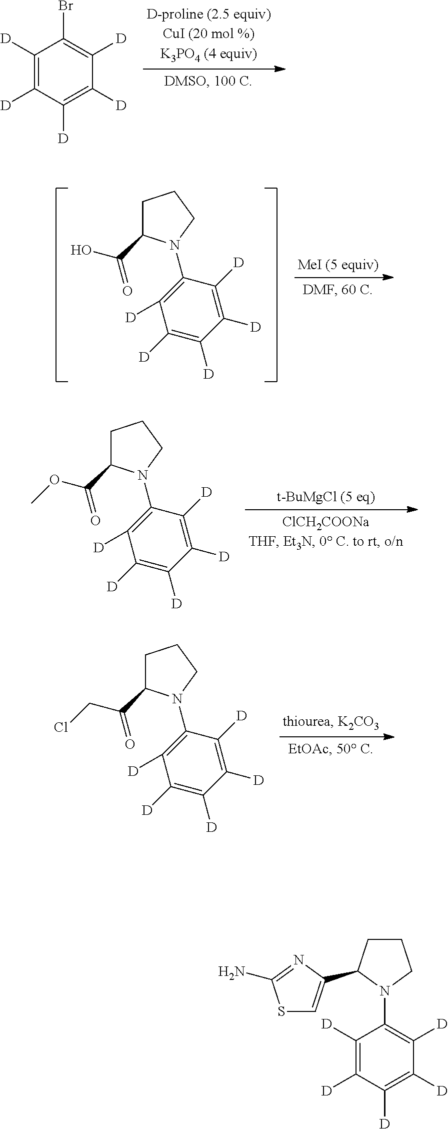

The Ullmann coupling and methylation were performed as described in route 13.

The chloroketone formation was performed as described in route 10.

A vial with stir bar was charged with chloroketone (244 mg, 1.07 mmol, 1.0 equiv), thiourea (89 mg, 1.17 mmol, 1.1 equiv) and K₂CO₃ (221 mg, 1.60 mmol, 1.5 equiv). EtOAc (5 mL, 0.2 M) was added, and the reaction mixture was stirred at 50 C for 3.5 h, until consumption of starting material was observed. The reaction mixture was cooled to room temperature, diluted with EtOAc (50 mL) and washed with saturated NaHCO₃ (2×50 mL). The combined aqueous layers were extracted with EtOAc, and the combined organic layers were dried over Na₂SO₄, filtered and concentrated in vacuo. The resulting crude material was purified via silica gel chromatography to yield the desired product.

### Route 29:

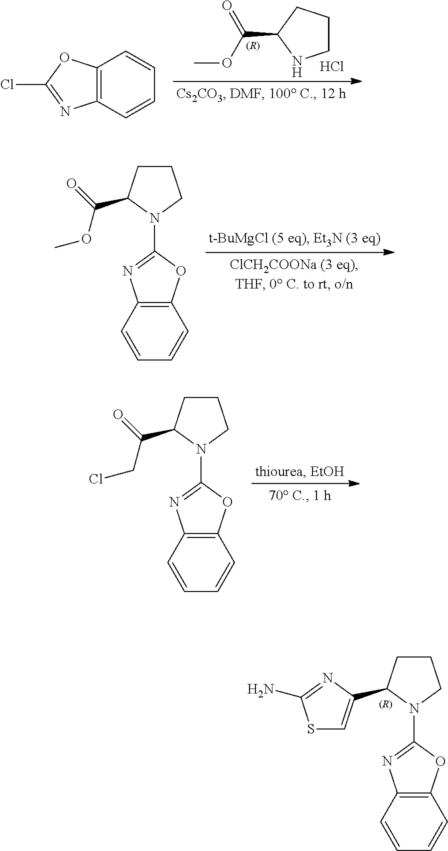

A 250 mL vial with stir bar was charged with 2-chloro-benzoxazole (1.00 g, 6.54 mmol, 1.00 equiv.), D-proline (1.88 g, 16.35 mmol, 2.50 equiv.) and degassed DMSO (60 mL, 0.08 M). CuI (250 mg, 1.31 mmol, 0.20 equiv.) and K₃PO₄ (5.53 g, 26.08 mmol, 4.00 equiv.) were added. The vial was evacuated, backflushed with nitrogen, and capped. The reaction mixture was stirred at 100° C. overnight. The next morning, the reaction mixture was cooled to room temperature, and Mel (0.81 mL, 13.03 mmol, 2.00 equiv.) was added. The reaction mixture was subsequently stirred at 60° C. for 1 h. The mixture was cooled to room temperature and poured into EtOAc (500 mL). The resulting solution was washed with brine (3×400 mL). The combined organic layers were dried over Na₂SO₄, filtered and concentrated in vacuo. The resulting crude material was purified via silica gel chromatography to yield the desired product.

The chloroketone formation was performed as described in route 10.

The condensation step was performed as described in route 1.

### Route 30:

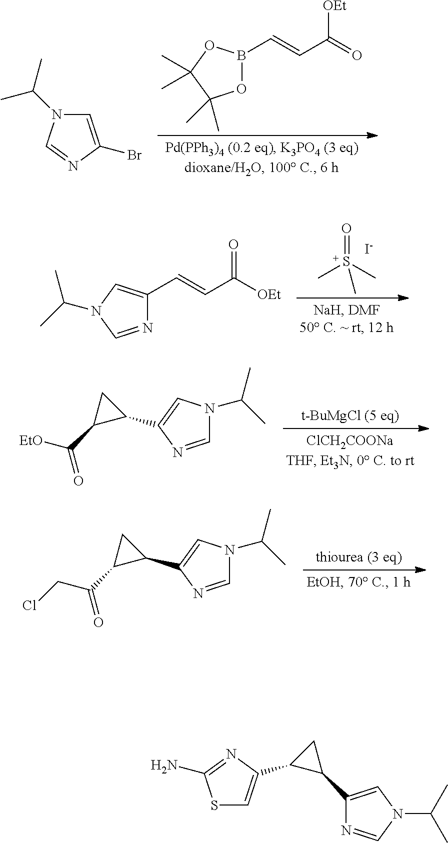

A 100 mL vial with stir bar was charged with 4-bromo-1-isopropylimidazole (1.00 g, 5.29 mmol, 1.00 equiv.), ethyl (2Z)-3-(4,4,5,5-tetramethyl-1,3,2-dioxaborolan-2-yl)prop-2-enoate (2.39 g, 10.58 mmol, 2.00 equiv.), K₃PO₄ (3.37 g, 15.87 mmol, 3.00 equiv.), Pd(PPh₃)₄ (1.22 g, 1.06 mmol, 0.20 equiv.), dioxane (20 mL, 0.09 M) and H₂O (4 mL). The vial was evacuated and backflushed with nitrogen. The vial was capped and placed in an 100° C. bath, and the reaction mixture was allowed to stir at 100° C. for 6 h. The reaction mixture was cooled to room temperature. The reaction mixture was poured into EtOAc (120 mL) and washed with brine (2×80 mL). The combined organic layers were dried over Na₂SO₄, filtered and concentrated in vacuo. The resulting crude material was purified via RP chromatography to yield the desired product.

A 100 mL round bottom flask with stir bar was charged with trimethylsulfoxonium iodide (475.52 mg, 2.16 mmol, 1.50 equiv.) and DMF (10 mL, 0.2 M). NaH (60 wt % in mineral oil, 41.48 mg, 1.73 mmol, 1.20 equiv.) was slowly added, and the reaction mixture was allowed to stir at 50° C. for 45 min. The reaction mixture was cooled to room temperature. Ethyl (2E)-3-(1-isopropylimidazol-4-yl)prop-2-enoate (300 mg, 1.44 mmol, 1.00 equiv.) in dry DMF (3 mL, 0.11 M) was added dropwise, and the reaction mixture was allowed to stir at 25° C. overnight. The next morning, the reaction mixture was quenched by H₂O (70 mL). The mixture was extracted with EtOAc (3×50 mL) and the combined organic layers were washed with brine (2×50 mL). The combined organic layers were dried over Na₂SO₄, filtered and concentrated in vacuo. The resulting crude material was purified via RP chromatography to yield the desired product.

The chloroketone formation was performed as described in route 10.

The condensation step was performed as described in route 1.

### Route 31:

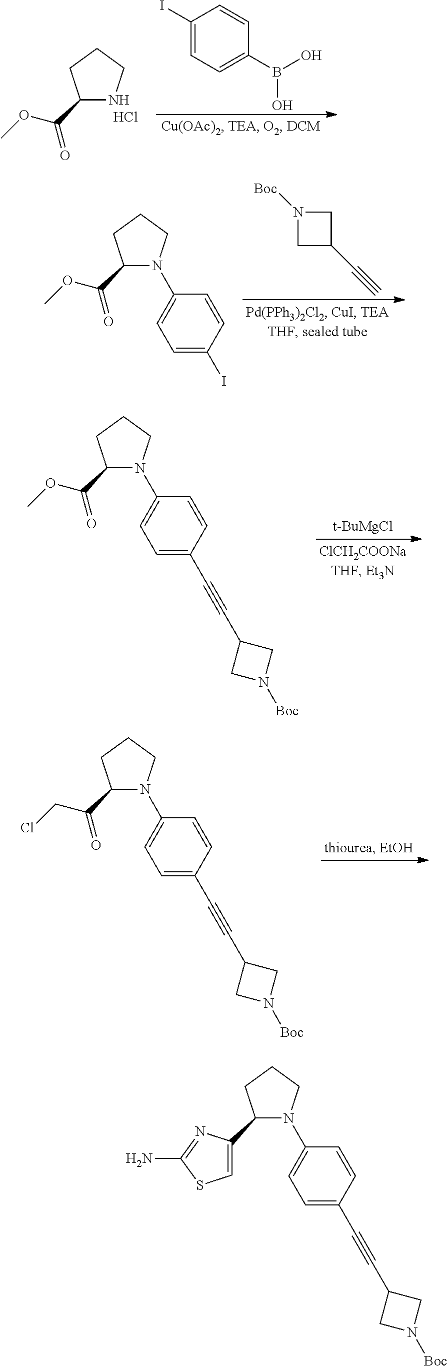

The Chan-Lam coupling step was performed as described in route 12.

A 50 mL sealed tube with stir bar was charged with methyl (2R)-1-(4-iodophenyl)pyrrolidine-2-carboxylate (304.8 mg, 0.92 mmol, 1.00 equiv.), tert-butyl 3-ethynylazetidine-1-carboxylate (1.31 g, 7.25 mmol, 1.50 equiv.), TEA (2.0 mL, 14.50 mmol, 3.00 equiv.), Pd(PPh₃)₂Cl₂ (339.13 mg, 0.48 mmol, 0.10 equiv.), CuI (184.04 mg, 0.97 mmol, 0.20 equiv.) and THF (25 mL, 0.04 M). The flask was evacuated and flushed with nitrogen. The vial was capped and placed in an 70° C. bath. The reaction mixture was stirred at 70° C. for 3 h. The reaction mixture was cooled to room temperature. The reaction mixture was poured into EtOAc (150 mL) and washed with H₂O (1×120 mL), followed by brine (2×120 mL). The organic layer was dried over Na₂SO₄, filtered and concentrated in vacuo. The resulting crude material was purified via silica gel chromatography to yield the desired product.

The chloroketone formation was performed as described in route 10.

The condensation step was performed as described in route 1.

Acid Synthesis

Pyrrole Alkylations

### Route 1:

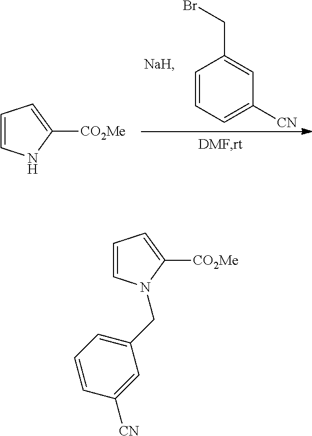 [SMILES: COC(=O)c1ccc[nH]1 ; N#Cc1cccc(CBr)c1 ; COC(=O)c1cccn1Cc1cccc(C#N)c1]

A 100 mL roundbottom flask with stir bar was charged with NaH (60 wt % in mineral oil, 245 mg, 6.12 mmol, 1.2 equiv) and DMF (15 mL). Methyl 1H-pyrrole-2-carboxylate (702 mg, 5.61 mmol, 1.1 equiv) was slowly added to the slurry, and the reaction mixture was allowed to stir at room temperature for 1 h. 3-(bromomethyl)benzonitrile (1.00 g, 5.10 mmol, 1.0 equiv) was added, and the reaction mixture was allowed to stir at room temperature overnight. The next morning, the reaction mixture was diluted with EtOAc (200 mL) and washed with water (2×200 mL). The combined organic layers were extracted with EtOAc (1×100 mL). The combined organic layers were dried over Na₂SO₄, filtered and concentrated in vacuo. The resulting crude material was purified via silica gel chromatography to yield the desired product.

### Route 2:

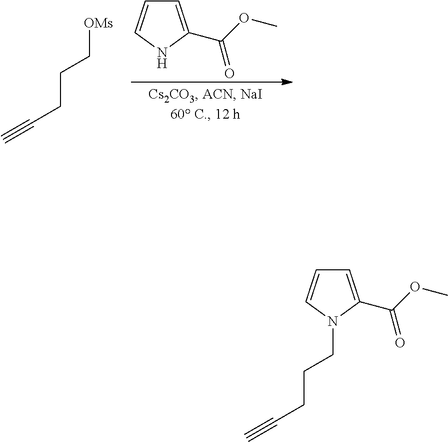

A 100 mL roundbottom flask with stir bar was charged with pent-4-yn-1-yl methanesulfonate (600.00 mg, 3.70 mmol, 1.00 equiv), methyl pyrrole-2-carboxylate (555.43 mg, 4.44 mmol, 1.20 equiv), Cs₂CO₃ (3.62 g, 11.10 mmol, 3.00 equiv), NaI (110.90 mg, 0.74 mmol, 0.20 equiv) in ACN (20 mL). The resulting solution was stirred at 60° C. overnight. The next morning, the reaction mixture was cooled to room temperature and diluted with EtOAc (100 mL) and washed with water (2×100 mL). The organic layers were dried over Na₂SO₄, filtered and concentrated in vacuo. The resulting crude material was purified via silica gel chromatography to yield the desired product.

### Route 3:

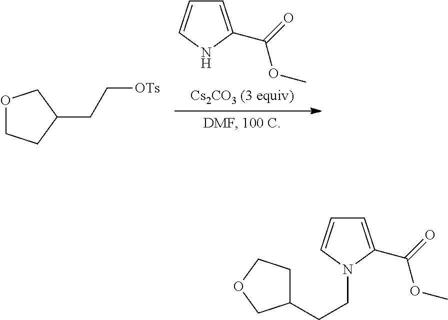

A vial with stir bar was charged with methyl 1H-pyrrole-2-carboxylate (23 mg, 0.18 mmol, 1.1 equiv), tosylate (45 mg, 0.17 mmol, 1.0 equiv) and Cs₂CO₃ (160 mg, 0.50 mmol, 3.0 equiv). DMF (1 mL) was added, and the reaction mixture was allowed to stir at 100 C overnight. The next morning, the reaction mixture was cooled to room temperature and diluted with EtOAc (50 mL). The organic layer was washed with water (2×50 mL), and the combined aqueous layers were extracted with EtOAc (1×50 mL). The combined organic layers were dried over Na₂SO₄, filtered and concentrated in vacuo. The resulting crude material was purified via silica gel chromatography to yield the desired product.

### Route 4:

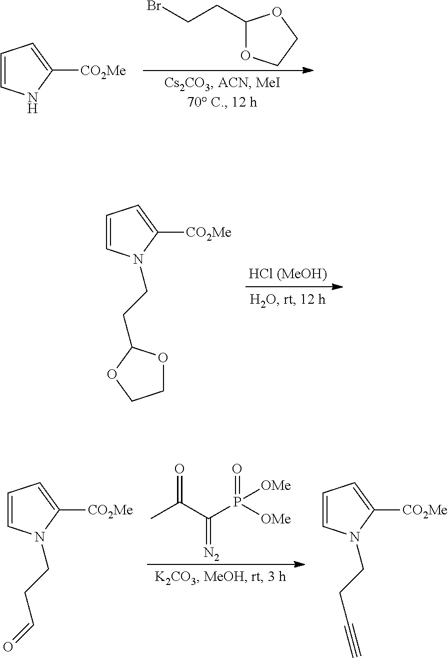

The alkylation was performed as described in route 2.

A 100 mL vial with stir bar was charged with methyl 1-[2-(1,3-dioxolan-2-yl)ethyl]pyrrole-2-carboxylate (200.00 mg, 0.89 mmol, 1.00 equiv), HCl (aq) (4.00 mL, 4M, 17.98 equiv) and THF (4 mL). The vial was capped and placed in a 25° C. bath. The reaction mixture was stirred at 25° C. for 2 h. The pH value of the solution was adjusted to 8 with NaHCO₃(aq). The resulting solution was extracted with ethyl acetate (2×20 mL) and washed with brine (1×20 mL). The combined organic layers were dried over Na₂SO₄, filtered and concentrated in vacuo. The resulting crude material was used directly for next step.

A 100 mL vial with stir bar was charged with methyl 1-(3-oxopropyl)pyrrole-2-carboxylate (200.00 mg, 1.10 mmol, 1.00 equiv), seyferth-gilbert homologation (318.08 mg, 1.66 mmol, 1.50 equiv), K₂CO₃ (305.10 mg, 2.21 mmol, 2.00 equiv) and MeOH (5.00 mL) under nitrogen atmosphere. The vial was capped and placed in a 25° C. bath. The reaction mixture was stirred at 25° C. for 2 h. The reaction mixture was quenched by H₂O (20 mL). The resulting solution was extracted with ethyl acetate (2×20 mL) and washed with brine (1×20 mL). The combined organic layers were dried over Na₂SO₄, filtered and concentrated in vacuo. The resulting crude material was purified via silica gel chromatography to yield the desired product.

### Route 5:

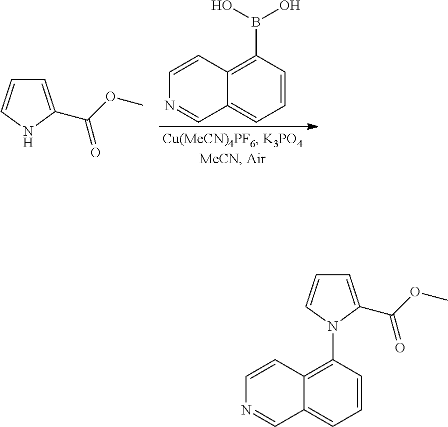

A 100 mL vial with stir bar was charged with methyl pyrrole-2-carboxylate (100.00 mg, 0.80 mmol, 1.00 equiv.), isoquinolin-5-ylboronic acid (414.73 mg, 2.40 mmol, 3.00 equiv.), K₃PO₄ (508.92 mg, 2.40 mmol, 3.00 equiv.), Cu(MeCN)₄PF₆ (148.65 mg, 0.40 mmol, 0.50 equiv.) and ACN (15 mL, 0.05 M). The vial was capped and placed in a 25° C. bath. The reaction mixture was stirred at 25° C. for 12 h. The reaction mixture was poured into EtOAc (80 mL) and washed with H₂O (1×40 mL) and brine (1×40 mL). The organic layer was dried over Na₂SO₄, filtered and concentrated in vacuo. The resulting crude material was purified via silica gel chromatography to yield the desired product.

### Route 6:

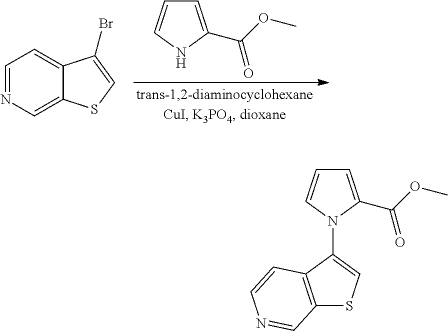

A 50 mL vial with stir bar was charged with 3-bromothieno[2,3-c]pyridine (100 mg, 0.47 mmol, 1.00 equiv.), methyl pyrrole-2-carboxylate (70.14 mg, 0.56 mmol, 1.20 equiv.), CuI (17.79 mg, 0.09 mmol, 0.20 equiv.), (1R,2R)-cyclohexane-1,2-diamine (10.67 mg, 0.09 mmol, 0.20 equiv.), K₃PO₄ (297.46 mg, 1.40 mmol, 3.00 equiv.) and dioxane (8 mL, 0.06 M). The flask was evacuated and flushed with nitrogen. The vial was capped and placed in an 80° C. bath. The reaction mixture was stirred at 80° C. overnight. The next morning, the reaction mixture was cooled to room temperature. The reaction mixture was poured into EtOAc (50 mL) and washed with H₂O (1×30 mL), followed by brine (1×30 mL). The organic layer was then dried over Na₂SO₄, filtered and concentrated in vacuo. The resulting crude material was purified via silica gel chromatography to yield the desired product.

The following compounds were prepared via a similar method:

| | Procedure | |
|---|---|---|
| | used | Compound name |
| C49 | 1 | methyl 1-(3-cyanobenzyl)-1H-pyrrole-2-carboxylate |
| C50 | 1 | methyl 1-(4-cyanobenzyl)-1H-pyrrole-2-carboxylate |
| C27 and C31 | 2 | methyl 1-(pent-4-yn-1-yl)-1H-pyrrole-2-carboxylate |
| C30 and C26 | 2 | methyl 1-(hex-5-yn-1-yl)-1H-pyrrole-2-carboxylate |
| C57 | 2 | methyl 1-((3,6-dihydro-2H-pyran-4-yl)methyl)-1H-pyrrole- |
| | | 2-carboxylate |
| C21 | 2 | methyl 1-((4,4-difluorocyclohexyl)methyl)-1H-pyrrole-2- |
| | | carboxylate |
| C29 | 2 | methyl 1-(prop-2-yn-1-yl)-1H-pyrrole-2-carboxylate |
| C37 | 2 | methyl 1-((1,1-dioxidotetrahydro-2H-thiopyran-4- |
| | | yl)methyl)-1H-pyrrole-2-carboxylate |
| C23 | 2 | methyl 1-(4-methoxybutyl)-1H-pyrrole-2-carboxylate |
| C39 | 2 | methyl 1-(cyclohexylmethyl)-1H-pyrrole-2-carboxylate |
| C3 | 3 | methyl 1-(2-(tetrahydrofuran-3-yl)ethyl)-1H-pyrrole-2- |
| | | carboxylate |
| C18 | 2 | methyl 1-(((1R,2S)-2-(cyanomethyl)cyclobutyl)methyl)-1H- |
| | | pyrrole-2-carboxylate |
| C19 | 2 | methyl 1-((3-(cyanomethyl)oxetan-3-yl)methyl)-1H- |
| | | pyrrole-2-carboxylate |
| C20 | 2 | methyl 1-(3-cyano-2-methylpropyl)-1H-pyrrole-2- |
| | | carboxylate |
| C11 and C60 | 2 | benzyl 1-(5-(tert-butoxy)-5-oxopentyl)-1H-pyrrole-2- |
| | | carboxylate |
| C12 and C59 | 2 | benzyl 1-(3-(tert-butoxy)-3-oxopropyl)-1H-pyrrole-2- |
| | | carboxylate |
| C6 | 2 | methyl 1-((2-cyanocyclopropyl)methyl)-1H-pyrrole-2- |
| | | carboxylate |
| C9 | 2 | methyl 1-((1-(cyanomethyl)cyclobutyl)methyl)-1H-pyrrole- |
| | | 2-carboxylate |
| Common intermediate | 1 | methyl 1-((tetrahydro-2H-pyran-4-yl)methyl)-1H-pyrrole-2- |
| | | carboxylate |
| C13 and C61 | 2 | benzyl 1-(4-(tert-butoxy)-4-oxobutyl)-1H-pyrrole-2- |
| | | carboxylate |
| C4, C1, and C58 | 2 | methyl 1-(4-cyanobutyl)-1H-pyrrole-2-carboxylate |
| C14, C2, and C7 | 2 | methyl 1-(3-cyanopropyl)-1H-pyrrole-2-carboxylate |
| C16, C32, and C15 | 2 | methyl 1-(2-cyanoethyl)-1H-pyrrole-2-carboxylate |
| C10 | 2 | methyl 1-((6-methoxypyridin-3-yl)methyl)-1H-pyrrole-2- |
| | | carboxylate |
| C8 | 2 | methyl 1-((1-(cyanomethyl)cyclopropyl)methyl)-1H- |
| | | pyrrole-2-carboxylate |
| C56 | 1 | methyl 1-((tetrahydrofuran-3-yl)methyl)-1H-pyrrole-2- |
| | | carboxylate |
| C5 | 1 | methyl 1-benzyl-1H-pyrrole-2-carboxylate |
| Common intermediate | 2 | methyl 1-((2-fluoropyridin-4-yl)methyl)-1H-pyrrole-2- |
| | | carboxylate |
| E27 | 2 | methyl 1-((3-fluoropyridin-4-yl)methyl)-1H-pyrrole-2- |
| | | carboxylate |
| E20 | 1 | methyl 1-(pyridin-3-ylmethyl)-1H-pyrrole-2-carboxylate |
| E21 | 2 | methyl 1-(2-(pyridin-3-yl)ethyl)-1H-pyrrole-2-carboxylate |
| E59, E22, and E58 | 1 | methyl 1-((3-bromopyridin-4-yl)methyl)-1H-pyrrole-2- |
| | | carboxylate |
| E60 and E23 | 1 | methyl 1-((2-bromopyridin-4-yl)methyl)-1H-pyrrole-2- |
| | | carboxylate |
| E13 | 2 | methyl 1-(1-(pyridin-4-yl)ethyl)-1H-pyrrole-2-carboxylate |
| C62 | 4 | methyl 1-(but-3-yn-1-yl)-1H-pyrrole-2-carboxylate |
| E10 | 2 | methyl 1-(3-(pyridin-3-yl)propyl)-1H-pyrrole-2-carboxylate |
| Common intermediate | 1 | methyl 1-(pyridin-4-ylmethyl)-1H-pyrrole-2-carboxylate |
| C84 | 2 | methyl 1-(oxetan-3-ylmethyl)-1H-pyrrole-2-carboxylate |
| C86 | 1 | methyl 1-(2-cyanobenzyl)-1H-pyrrole-2-carboxylate |
| C87 and C117 | 2 | methyl (S)-1-(3-cyano-2-methylpropyl)-1H-pyrrole-2- |
| | | carboxylate |
| A52 | 1 | methyl 1-((2-methylpyridin-4-yl)methyl)-1H-pyrrole-2- |
| | | carboxylate |
| A53 | 1 | methyl 1-((3-methylpyridin-4-yl)methyl)-1H-pyrrole-2- |
| | | carboxylate |
| C88 | 2 | methyl (R)-1-(3-cyano-2-methylpropyl)-1H-pyrrole-2- |
| | | carboxylate |
| C90 | 2 | methyl 1-(4-cyanobutan-2-yl)-1H-pyrrole-2-carboxylate |
| C92 | 3 | methyl 1-(3-(tetrahydrofuran-3-yl)propyl)-1H-pyrrole-2- |
| | | carboxylate |
| C94 | 1 | methyl 1-((3,3-difluorocyclobutyl)methyl)-1H-pyrrole-2- |
| | | carboxylate |
| C99 | 3 | methyl 1-(2-(oxetan-3-yl)ethyl)-1H-pyrrole-2-carboxylate |
| A60, A122, and B87 | 2 | benzyl 1-((2,6-difluoropyridin-4-yl)methyl)-1H-pyrrole-2- |
| | | carboxylate |
| C102 | 2 | methyl 1-(pyridazin-4-ylmethyl)-1H-pyrrole-2-carboxylate |
| C106 | 3 | methyl 1-((3-cyanocyclobutyl)methyl)-1H-pyrrole-2- |
| | | carboxylate |
| C111 | 5 | methyl 1-(isoquinolin-5-yl)-1H-pyrrole-2-carboxylate |
| C113 | 2 | methyl 1-(2-(cyanomethyl)butyl)-1H-pyrrole-2-carboxylate |
| C115 | 6 | methyl 1-(thieno[2,3-c]pyridin-3-yl)-1H-pyrrole-2- |
| | | carboxylate |
| B63 and B66 | 2 | methyl 1-(pyridin-3-ylmethyl)-1H-pyrrole-2-carboxylate |
| A115 | 1 | ethyl 4-methyl-1-(pyridin-4-ylmethyl)-1H-pyrrole-2- |
| | | carboxylate |
| C116 | 3 | methyl 1-(3-(oxetan-3-yl)propyl)-1H-pyrrole-2-carboxylate |
| A149 and A152 | 2 | methyl 4-cyano-1-((2-fluoropyridin-4-yl)methyl)-1H- |
| | | pyrrole-2-carboxylate |
| Common intermediate | 1 | ethyl 1-((2-fluoropyridin-4-yl)methyl)-4-methyl-1H-pyrrole- |
| | | 2-carboxylate |
| C118 and D8 | 1 | methyl 1-(3-fluorobenzyl)-1H-pyrrole-2-carboxylate |
| C119 | 1 | methyl 1-(4-fluorobenzyl)-1H-pyrrole-2-carboxylate |
| C120 | 2 | methyl 1-(3-methylbenzyl)-1H-pyrrole-2-carboxylate |
| C121 | 2 | methyl 1-(3-bromobenzyl)-1H-pyrrole-2-carboxylate |
| C122 | 2 | methyl 1-(3-chlorobenzyl)-1H-pyrrole-2-carboxylate |
| C123 | 2 | benzyl 1-((3,3-difluorocyclopentyl)methyl)-1H-pyrrole-2- |
| | | carboxylate |
| A185 | 2 | methyl 4-bromo-1-((2-fluoropyridin-4-yl)methyl)-1H- |
| | | pyrrole-2-carboxylate |
| C124 | 2 | methyl 1-(cyclopentylmethyl)-1H-pyrrole-2-carboxylate |
| A189 | 2 | methyl 4-fluoro-1-((2-fluoropyridin-4-yl)methyl)-1H-pyrrole- |
| | | 2-carboxylate |
| A191 | 2 | methyl 4-chloro-1-((2-fluoropyridin-4-yl)methyl)-1H- |
| | | pyrrole-2-carboxylate |
| C125 | 2 | methyl 1-(furan-3-ylmethyl)-1H-pyrrole-2-carboxylate |
| C126 | 2 | methyl 1-(furan-2-ylmethyl)-1H-pyrrole-2-carboxylate |

Saponifications

### Route 1:

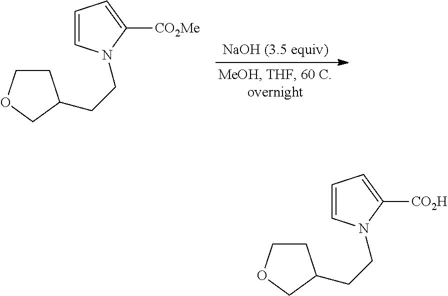 [SMILES: COC(=O)c1cccn1CCC1CCOC1 ; O=C(O)c1cccn1CCC1CCOC1]

A vial with stir bar was charged with methyl ester (27 mg, 0.12 mmol, 1.0 equiv), MeOH (0.5 mL) and THF (0.5 mL). Aqueous NaOH (5 M, 85 uL, 0.42 mmol, 3.5 equiv) was added, and the reaction mixture was stirred at 60 C overnight. The next morning, the reaction mixture was diluted with EtOAc (50 mL) and water (25 mL). The organic layer was removed, and the aqueous layer was acidified with 1 M HCl. The aqueous layer was extracted with EtOAc (50 mL). The organic layer was dried over Na₂SO₄, filtered and concentrated in vacuo. The resulting crude material was used in the next step without further purification.

### Route 2:

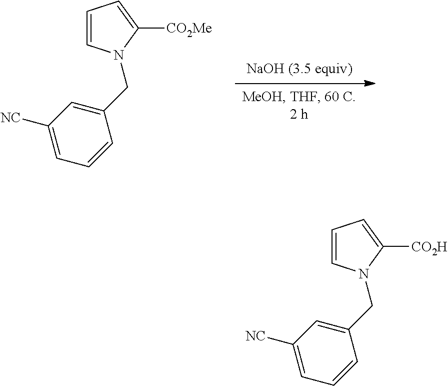 [SMILES: COC(=O)c1cccn1Cc1cccc(C#N)c1 ; "MeOH, THF, 60 C.2 h" ; "NaOH (3.5 equiv)" ; N#Cc1cccc(Cn2cccc2C(=O)O)c1]

A vial with stir bar was charged with methyl ester (300 mg, 1.25 mmol, 1.0 equiv), MeOH (2.5 mL) and THF (2.5 mL). Aqueous NaOH (5 M, 0.874 mL, 4.37 mmol, 3.5 equiv) was added, and the reaction mixture was stirred at 60 C for 2 h. After 2 h, the reaction mixture was cool to room temperature, and the volatile solvents were removed in vacuo. The resulting aqueous slurry was acidified with 1 M HCl, and the precipitate was filtered and washed. The crude precipitated material was used in the next step without further purification

### Route 3:

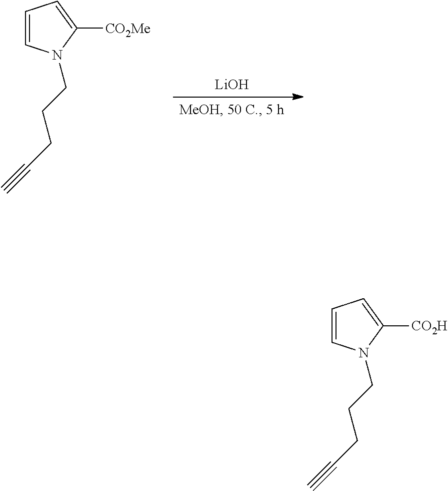

A 50 mL vial with stir bar was charged with methyl 1-(pent-4-yn-1-yl)pyrrole-2-carboxylate (300.00 mg, 1.57 mmol, 1.00 equiv), LiOH (187.86 mg, 7.84 mmol, 5.00 equiv) and MeOH (6.00 mL), H₂O (2.00 mL). The vial was capped and placed in a 40° C. bath. The reaction mixture was stirred at 40° C. for 5 h. The reaction mixture was cooled to room temperature, the pH value of the solution was adjusted to 7 with HCl (1 mol/L). The resulting solution was extracted with (3×30 mL) of ethyl acetate. The organic layer was then dried over Na₂SO₄, filtered and concentrated in vacuo. The crude precipitated material was used in the next step without further purification.

### Route 4:

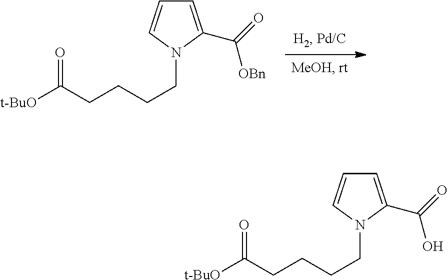 [SMILES: CC(C)(C)OC(=O)CCCCn1cccc1C(=O)OCc1ccccc1 ; "H2, Pd/C" ; "MeOH, rt" ; CC(C)(C)OC(=O)CCCCn1cccc1C(=O)O]

A 100 mL vial with stir bar was charged with benzyl 1-[5-(tert-butoxy)-5-oxopentyl]pyrrole-2-carboxylate (1.00 g, 2.80 mmol, 1.00 equiv) and Pd/C (10%, 595.3 mg, 5.60 mmol, 2.00 equiv) in MeOH (10 mL) under nitrogen atmosphere. The flask was then vacuumed and flushed with hydrogen. The reaction mixture was hydrogenated at room temperature for 3 hours under hydrogen atmosphere using a hydrogen balloon. Then the reaction mixture was filtered through a celite pad and the filtrate was concentrated under reduced pressure. The crude precipitated material was used in the next step without further purification.

### Route 5:

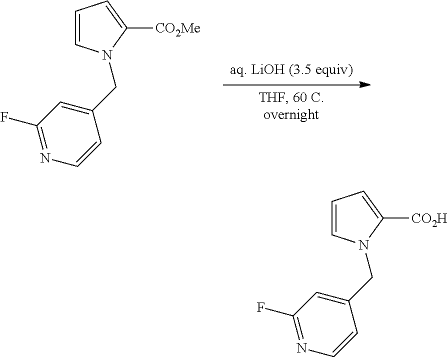 [SMILES: COC(=O)c1cccn1Cc1ccnc(F)c1 ; "THF, 60 C.overnight" ; "aq. LiOH (3.5 equiv)" ; O=C(O)c1cccn1Cc1ccnc(F)c1]

A 40 mL vial with stir bar was charged with ester (2.0 g, 8.5 mmol, 1.0 equiv.) and THF (20 mL, 0.3 M). LiOH (5 M in water, 6.0 mL, 30 mmol, 3.5 equiv.) was added, and the vial was capped and allowed to stir at 60° C. overnight. The next morning, the reaction mixture was concentrated in vacuo, and 1 M HCl was added to bring the pH of the solution to ˜4. The resulting precipitate was filtered, washed with water, and used in the next step without further purification.

### Route 6:

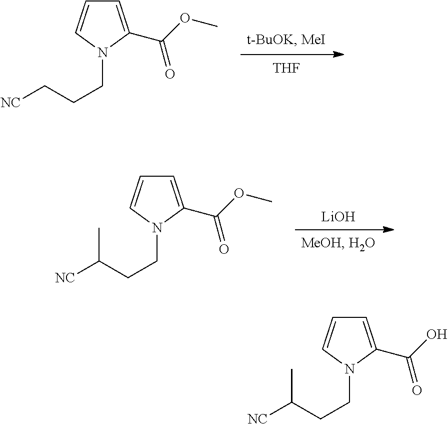

A 100 mL vial with stir bar was charged with methyl 1-(3-cyanopropyl)pyrrole-2-carboxylate (576.00 mg, 3.00 mmol, 1.00 equiv.), t-BuOK (672.00 mg, 5.99 mmol, 2.00 equiv.) and THF (15 mL, 0.20 M) at 0° C. The flask was evacuated and flushed with nitrogen. CH₃I (0.56 mL, 9.02 mmol, 3.00 equiv.) was added at 0° C. The vial was capped and placed in a 25° C. bath. The reaction mixture was stirred at 25° C. overnight. The next morning, the reaction mixture was poured into EtOAc (80 ml) and washed with H₂ (1×40 mL), followed by brine (1×40 mL). The organic layer was then dried over Na₂SO₄, filtered and concentrated in vacuo. The resulting crude material was purified via silica gel chromatography to yield the desired product.

The saponification was performed as described in route 3.

The following compounds were prepared via a similar method:

| | Procedure | |
|---|---|---|
| | used | Compound name |
| C49 | 2 | 1-(3-cyanobenzyl)-1H-pyrrole-2-carboxylic acid |
| C50 | 2 | 1-(4-cyanobenzyl)-1H-pyrrole-2-carboxylic acid |
| C27 and C31 | 3 | 1-(pent-4-yn-1-yl)-1H-pyrrole-2-carboxylic acid |
| C30 and C26 | 3 | 1-(hex-5-yn-1-yl)-1H-pyrrole-2-carboxylic acid |
| C62 | 3 | 1-(but-3-yn-1-yl)-1H-pyrrole-2-carboxylic acid |
| C57 | 3 | 1-((3,6-dihydro-2H-pyran-4-yl)methyl)-1H-pyrrole-2-carboxylic acid |
| C21 | 3 | 1-((4,4-difluorocyclohexyl)methyl)-1H-pyrrole-2-carboxylic acid |
| C33 and C29 | 3 | 1-(prop-2-yn-1-yl)-1H-pyrrole-2-carboxylic acid |
| C37 | 3 | 1-((1,1-dioxidotetrahydro-2H-thiopyran-4-yl)methyl)-1H-pyrrole-2- |
| | | carboxylic acid |
| C23 | 3 | 1-(4-methoxybutyl)-1H-pyrrole-2-carboxylic acid |
| C39 | 3 | 1-(cyclohexylmethyl)-1H-pyrrole-2-carboxylic acid |
| C3 | 1 | 1-(2-(tetrahydrofuran-3-yl)ethyl)-1H-pyrrole-2-carboxylic acid |
| C18 | 3 | 1-(((1R,2S)-2-(cyanomethyl)cyclobutyl)methyl)-1H-pyrrole-2- |
| | | carboxylic acid |
| C19 | 3 | 1-((3-(cyanomethyl)oxetan-3-yl)methyl)-1H-pyrrole-2-carboxylic acid |
| C20 | 3 | 1-(3-cyano-2-methylpropyl)-1H-pyrrole-2-carboxylic acid |
| C11 and C60 | 4 | 1-(5-(tert-butoxy)-5-oxopentyl)-1H-pyrrole-2-carboxylic acid |
| C12 and C59 | 4 | 1-(3-(tert-butoxy)-3-oxopropyl)-1H-pyrrole-2-carboxylic acid |
| C6 | 3 | 1-((2-cyanocyclopropyl)methyl)-1H-pyrrole-2-carboxylic acid |
| C9 | 3 | 1-((1-(cyanomethyl)cyclobutyl)methyl)-1H-pyrrole-2-carboxylic acid |
| C85, C91, D2, | 1 | 1-((tetrahydro-2H-pyran-4-yl)methyl)-1H-pyrrole-2-carboxylic acid |
| C96, C103, | | |
| C104, C105, | | |
| C107, D3, D4, | | |
| C110, D5, D6, | | |
| and D7 | | |
| C13 and C61 | 4 | 1-(4-(tert-butoxy)-4-oxobutyl)-1H-pyrrole-2-carboxylic acid |
| C4, C1, and C58 | 3 | 1-(4-cyanobutyl)-1H-pyrrole-2-carboxylic acid |
| C14, C2, and C7 | 3 | 1-(3-cyanopropyl)-1H-pyrrole-2-carboxylic acid |
| C16, C32, and | 3 | 1-(2-cyanoethyl)-1H-pyrrole-2-carboxylic acid |
| C15 | | |
| C10 | 3 | 1-((6-methoxypyridin-3-yl)methyl)-1H-pyrrole-2-carboxylic acid |
| C8 | 3 | 1-((1-(cyanomethyl)cyclopropyl)methyl)-1H-pyrrole-2-carboxylic acid |
| C56 | 1 | 1-((tetrahydrofuran-3-yl)methyl)-1H-pyrrole-2-carboxylic acid |
| C5 | 1 | 1-benzyl-1H-pyrrole-2-carboxylic acid |
| E26 and | 5 | 1-((2-fluoropyridin-4-yl)methyl)-1H-pyrrole-2-carboxylic acid |
| common | | |
| intermediate | | |
| E20 | 3 | 1-(pyridin-3-ylmethyl)-1H-pyrrole-2-carboxylic acid |
| E21 | 3 | 1-(2-(pyridin-3-yl)ethyl)-1H-pyrrole-2-carboxylic acid |
| E59, E22, and | 1 | 1-((3-bromopyridin-4-yl)methyl)-1H-pyrrole-2-carboxylic acid |
| E58 | | |
| E60 and E23 | 1 | 1-((2-bromopyridin-4-yl)methyl)-1H-pyrrole-2-carboxylic acid |
| E13 | 3 | 1-(1-(pyridin-4-yl)ethyl)-1H-pyrrole-2-carboxylic acid |
| E10 | 3 | 1-(3-(pyridin-3-yl)propyl)-1H-pyrrole-2-carboxylic acid |
| Common | 1 | 1-(pyridin-4-ylmethyl)-1H-pyrrole-2-carboxylic acid |
| intermediate | | |
| C84 | 1 | 1-(oxetan-3-ylmethyl)-1H-pyrrole-2-carboxylic acid |
| C86 | 2 | 1-(2-cyanobenzyl)-1H-pyrrole-2-carboxylic acid |
| C87 and C117 | 3 | (S)-1-(3-cyano-2-methylpropyl)-1H-pyrrole-2-carboxylic acid |
| A52 | 2 | 1-((2-methylpyridin-4-yl)methyl)-1H-pyrrole-2-carboxylic acid |
| A53 | 2 | 1-((3-methylpyridin-4-yl)methyl)-1H-pyrrole-2-carboxylic acid |
| C88 | 3 | (R)-1-(3-cyano-2-methylpropyl)-1H-pyrrole-2-carboxylic acid |
| C89 | 6 | 1-(3-cyanobutyl)-1H-pyrrole-2-carboxylic acid |
| C90 | 3 | 1-(4-cyanobutan-2-yl)-1H-pyrrole-2-carboxylic acid |
| C92 | 1 | 1-(3-(tetrahydrofuran-3-yl)propyl)-1H-pyrrole-2-carboxylic acid |
| C94 | 3 | 1-((3,3-difluorocyclobutyl)methyl)-1H-pyrrole-2-carboxylic acid |
| C99 | 1 | 1-(2-(oxetan-3-yl)ethyl)-1H-pyrrole-2-carboxylic acid |
| A60, B68, A122, | 4 | 1-((2,6-difluoropyridin-4-yl)methyl)-1H-pyrrole-2-carboxylic acid |
| and B87 | | |
| C102 | 3 | 1-(pyridazin-4-ylmethyl)-1H-pyrrole-2-carboxylic acid |
| C106 | 2 | 1-((3-cyanocyclobutyl)methyl)-1H-pyrrole-2-carboxylic acid |
| C111 | 3 | 1-(isoquinolin-5-yl)-1H-pyrrole-2-carboxylic acid |
| C113 | 3 | 1-(2-(cyanomethyl)butyl)-1H-pyrrole-2-carboxylic acid |
| C115 | 3 | 1-(thieno[2,3-c]pyridin-3-yl)-1H-pyrrole-2-carboxylic acid |
| B63 and B66 | 3 | 1-(pyridin-3-ylmethyl)-1H-pyrrole-2-carboxylic acid |
| A115 | 2 | 4-methyl-1-(pyridin-4-ylmethyl)-1H-pyrrole-2-carboxylic acid |
| C116 | 1 | 1-(3-(oxetan-3-yl)propyl)-1H-pyrrole-2-carboxylic acid |
| A149 and A152 | 5 | 4-cyano-1-((2-fluoropyridin-4-yl)methyl)-1H-pyrrole-2-carboxylic acid |
| Common | 5 | 1-((2-fluoropyridin-4-yl)methyl)-4-methyl-1H-pyrrole-2-carboxylic acid |
| intermediate | | |
| C118 and D8 | 3 | 1-(3-fluorobenzyl)-1H-pyrrole-2-carboxylic acid |
| C119 | 3 | 1-(4-fluorobenzyl)-1H-pyrrole-2-carboxylic acid |
| C120 | 3 | 1-(3-methylbenzyl)-1H-pyrrole-2-carboxylic acid |
| C121 | 3 | 1-(3-bromobenzyl)-1H-pyrrole-2-carboxylic acid |
| C122 | 3 | 1-(3-chlorobenzyl)-1H-pyrrole-2-carboxylic acid |
| C123 | 4 | 1-((3,3-difluorocyclopentyl)methyl)-1H-pyrrole-2-carboxylic acid |
| A185 | 5 | 4-bromo-1-((2-fluoropyridin-4-yl)methyl)-1H-pyrrole-2-carboxylic acid |
| C124 | 3 | 1-(cyclopentylmethyl)-1H-pyrrole-2-carboxylic acid |
| A189 | 3 | 4-fluoro-1-((2-fluoropyridin-4-yl)methyl)-1H-pyrrole-2-carboxylic acid |
| A191 | 3 | 4-chloro-1-((2-fluoropyridin-4-yl)methyl)-1H-pyrrole-2-carboxylic acid |
| C125 | 3 | 1-(furan-3-ylmethyl)-1H-pyrrole-2-carboxylic acid |
| C126 | 3 | 1-(furan-2-ylmethyl)-1H-pyrrole-2-carboxylic acid |

Alternative Routes

### Route 1:

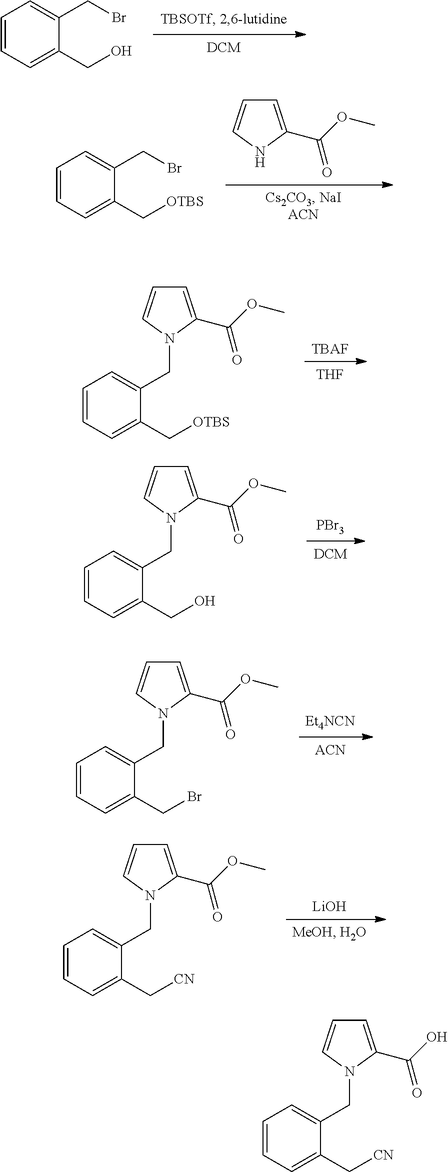

A 100 mL vial with stir bar was charged with [2-(bromomethyl)phenyl]methanol (500.00 mg, 2.49 mmol, 1.00 equiv.), 2,6-dimethylpyridine (0.58 mL, 4.97 mmol, 2.00 equiv.) and DCM (15 mL, 0.12 M). The flask was evacuated and flushed with nitrogen. TBSOTf (0.86 mL, 3.73 mmol, 1.50 equiv.) in DCM (5 mL) was added dropwise at 0° C. The vial was capped and placed in a 25° C. bath. The reaction mixture was stirred at 25° C. for 2 h. After 2 h, the reaction mixture was poured into DCM (50 mL) and washed with H₂O (1×50 mL), followed by brine (1×50 mL). The organic layer was then dried over Na₂SO₄, filtered and concentrated in vacuo. The crude product was used in the next step without further purification.

The alkylation was performed as described in alkylation route 2.

A 50 mL vial with stir bar was charged with methyl 1-(2-(((tert-butyldimethylsilyl)oxy)methyl)benzyl)-1H-pyrrole-2-carboxylate (500.00 mg, 1.39 mmol, 1.00 equiv.) and THF (10 mL, 0.14 M). TBAF (1 M in THF, 2.78 mL, 2.78 mmol, 2.00 equiv.) was added. The flask was evacuated and flushed with nitrogen. The vial was capped and placed in a 25° C. bath. The reaction mixture was stirred at 25° C. for 2 h. The reaction mixture was cooled to room temperature. The reaction mixture was quenched by H₂O (20 mL). The mixture was extracted with EtOAc (3×30 mL), and the combined organic layers were washed with brine (2×30 mL). The organic layer was then dried over Na₂SO₄, filtered and concentrated in vacuo. The crude product was used in the next step without further purification.

A 50 mL vial with stir bar was charged with methyl 1-(2-(hydroxymethyl)benzyl)-1H-pyrrole-2-carboxylate (350.00 mg, 1.43 mmol, 1.00 equiv.) and DCM (10 mL, 0.14 M). PBr3 (0.27 mL, 2.85 mmol, 2.00 equiv.) was added at 0° C. The vial was capped and placed in an 25° C. bath. The reaction mixture was stirred at 25° C. for 1 h. After 1 h, the reaction mixture was concentrated in vacuo. The resulting material was charged with DCM (50 mL) and washed with sat. NaHCO₃ (aq.) (1×30 mL), followed by brine (1×30 mL). The organic layer was then dried over Na2SO4, filtered and concentrated in vacuo. The crude product was used in the next step without further purification.

A 50 mL vial with stir bar was charged with methyl 1-(2-(bromomethyl)benzyl)-1H-pyrrole-2-carboxylate (350.00 mg, 1.14 mmol, 1.00 equiv), Et₄NCN (354.96 mg, 2.27 mmol, 2.00 equiv) and ACN (10 mL, 0.11 M). The flask was evacuated and flushed with nitrogen. The vial was capped and placed in a 25° C. bath. The reaction mixture was stirred at 25° C. overnight. The next morning, the reaction mixture was quenched by H₂O (30 mL). The mixture was extracted with EtOAc (3×30 mL), and the combined organic layers were washed with brine (2×30 mL). The organic layer was then dried over Na₂SO₄, filtered and concentrated in vacuo. The resulting crude material was purified via silica gel chromatography to yield the desired product.

The saponification was performed as described in saponification route 3.

### Route 2:

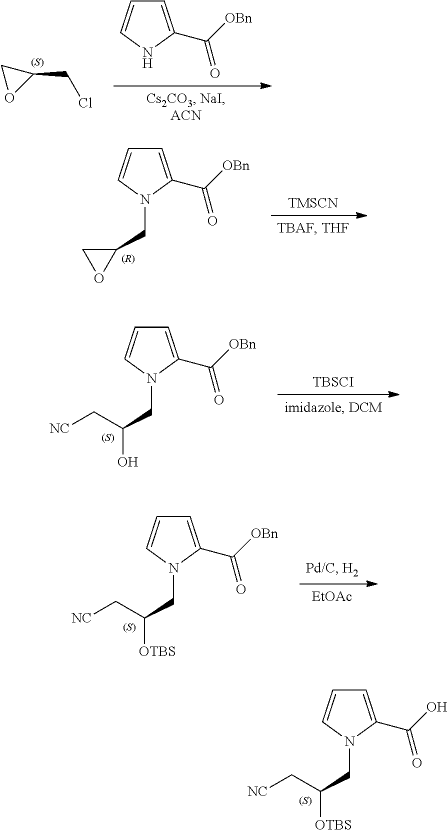

The alkylation was performed as described in alkylation route 2.

A 100 mL vial with stir bar was charged with benzyl (R)-1-(oxiran-2-ylmethyl)-1H-pyrrole-2-carboxylate (1.80 g, 7.00 mmol, 1.00 equiv.), TBAF hydrate (2.74 g, 10.49 mmol, 1.50 equiv.), TMSCN (1.3 mL, 10.49 mmol, 1.50 equiv.) and THF (40 mL, 0.18 M). The flask was evacuated and flushed with nitrogen. The vial was capped and placed in an 40° C. bath. The reaction mixture was stirred at 40° C. for 1 h. The reaction mixture was quenched by H₂O (80 mL). The mixture was extracted with DCM (3×100 mL), and the combined organic layers were washed with brine (1×100 mL). The organic layer was then dried over Na₂SO₄, filtered and concentrated in vacuo. The resulting crude material was purified via silica gel chromatography to yield the desired product.

A 100 mL vial with stir bar was charged with benzyl (S)-1-(3-cyano-2-hydroxypropyl)-1H-pyrrole-2-carboxylate (600 mg, 2.11 mmol, 1.00 equiv.), TBSCl (634 mg, 4.21 mmol, 2.00 equiv.), imidazole (430.5 mg, 6.32 mmol, 3.00 equiv.) and DCM (25 mL, 0.08 M). The flask was evacuated and flushed with nitrogen. The vial was capped and placed in an 25° C. bath. The reaction mixture was stirred at 25° C. for 4 h. The reaction mixture was poured into DCM (60 mL) and washed with H₂O (1×50 mL), followed by brine (1×50 mL). The organic layer was then dried over Na₂SO₄, filtered and concentrated in vacuo. The resulting crude material was purified via silica gel chromatography to yield the desired product.

The debenzylation was performed as described in saponification route 4.

The following compounds were prepared via a similar method:

| | Compound name |
|---|---|
| C97 | (S)-1-(3-cyano-2-methoxypropyl)-1H-pyrrole-2-carboxylic acid |
| C108 | (R)-1-(3-cyano-2-methoxypropyl)-1H-pyrrole-2-carboxylic acid |
| C100 | 1-((2S)-3-cyano-2-((tetrahydro-2H-pyran-2-yl)oxy)propyl)-1H-pyrrole-2-carboxylic acid |
| C112 | 1-((2R)-3-cyano-2-((tetrahydro-2H-pyran-2-yl)oxy)propyl)-1H-pyrrole-2-carboxylic acid |

Amide Couplings

### Route 1:

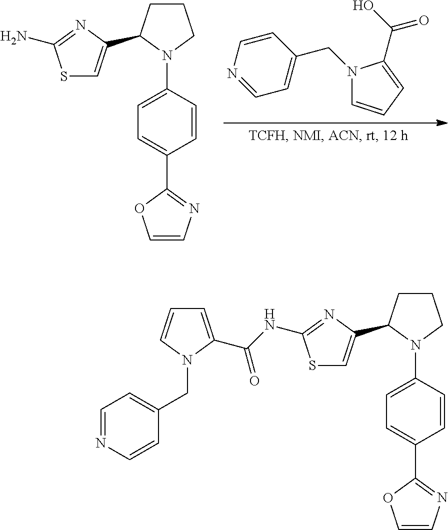

A 50 mL vial with stir bar was charged with 4-[(2R)-1-[4-(1,3-oxazol-2-yl)phenyl]pyrrolidin-2-yl]-1,3-thiazol-2-amine (150.00 mg, 0.48 mmol, 1.00 equiv), 1-(pyridin-4-ylmethyl)pyrrole-2-carboxylic acid (97.10 mg, 0.48 mmol, 1.00 equiv), NMI (137.98 mg, 1.68 mmol, 3.50 equiv) and ACN (5 mL) under nitrogen atmosphere, TCFH (154.93 mg, 0.55 mmol, 1.15 equiv) was added. The vial was capped and placed in a 50° C. bath. The reaction mixture was stirred at 50° C. for 4 h. The reaction mixture was cooled to room temperature. The reaction mixture was poured into DCM (50 mL) and washed with brine (2×50 mL), and the combined organic layers were dried over Na₂SO₄, filtered and concentrated in vacuo. The resulting crude material was purified via silica gel chromatography & Prep-HPLC or RP column to yield the desired product.

### Route 2:

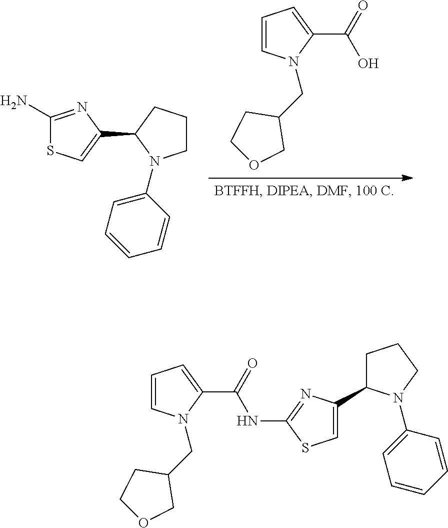

A vial with stir bar was charged with amine (81 mg, 0.33 mmol, 1.0 equiv), acid (71 mg, 0.36 mmol, 1.1 equiv), and BTFFH (110 mg, 0.36 mmol, 1.1. equiv). DMF (1 mL) and DIPEA (0.12 mL, 0.66 mmol, 2.0 equiv) were added. The vial was capped, and the reaction mixture was allowed to stir at 100 C overnight. The next morning, the reaction mixture was cooled to room temperature and diluted with EtOAc (50 mL). The reaction mixture was washed with a mixture of 1 M NaOH and brine (1:1, 2×50 mL). The combined aqueous layers were extracted with EtOAc (1×50 mL). The combined organic layers were dried over Na2SO4, filtered and concentrated in vacuo. The resulting crude material was purified via silica gel chromatography to yield the desired product.

The following compounds were prepared via a similar method:

| | | Observed | |
|---|---|---|---|
| | Procedure | molecular | |
| | used | ion | Compound name |
| A9 | 1 | 497 | (R)-N-(4-(1-(4-(oxazol-2-yl)phenyl)pyrrolidin-2-yl)thiazol-2-yl)-1-(pyridin-4- |
| | | | ylmethyl)-1H-pyrrole-2-carboxamide |
| C56 | 2 | 423 | N-(4-((R)-1-phenylpyrrolidin-2-yl)thiazol-2-yl)-1-((tetrahydrofuran-3- |
| | | | yl)methyl)-1H-pyrrole-2-carboxamide |
| A26 | 2 | 464 | (R)-N-(4-(1-(4-chlorophenyl)pyrrolidin-2-yl)thiazol-2-yl)-1-(pyridin-4- |
| | | | ylmethyl)-1H-pyrrole-2-carboxamide |
| Int for | 1 | 629 | tert-butyl (R)-4-(4-(2-(2-(1-(pyridin-4-ylmethyl)-1H-pyrrole-2- |
| A7, A8 | | | carboxamido)thiazol-4-yl)pyrrolidin-1-yl)phenoxy)piperidine-1-carboxylate |
| and A6 | | | |
| A25 | 1 | 448 | (R)-N-(4-(1-(4-fluorophenyl)pyrrolidin-2-yl)thiazol-2-yl)-1-(pyridin-4- |
| | | | ylmethyl)-1H-pyrrole-2-carboxamide |
| A22 | 1 | 458 | (R)-N-(4-(1-(4-ethylphenyl)pyrrolidin-2-yl)thiazol-2-yl)-1-(pyridin-4- |
| | | | ylmethyl)-1H-pyrrole-2-carboxamide |
| A23 | 1 | 472 | (R)-N-(4-(1-(4-isopropylphenyl)pyrrolidin-2-yl)thiazol-2-yl)-1-(pyridin-4- |
| | | | ylmethyl)-1H-pyrrole-2-carboxamide |
| A24 | 1 | 470 | (R)-N-(4-(1-(4-cyclopropylphenyl)pyrrolidin-2-yl)thiazol-2-yl)-1-(pyridin-4- |
| | | | ylmethyl)-1H-pyrrole-2-carboxamide |
| A5 | 1 | 488 | (R)-N-(4-(1-(4-isopropoxyphenyl)pyrrolidin-2-yl)thiazol-2-yl)-1-(pyridin-4- |
| | | | ylmethyl)-1H-pyrrole-2-carboxamide |
| A28 | 2 | 498 | (R)-1-(pyridin-4-ylmethyl)-N-(4-(1-(4-(trifluoromethyl)phenyl)pyrrolidin-2- |
| | | | yl)thiazol-2-yl)-1H-pyrrole-2-carboxamide |
| A21 | 2 | 444 | (R)-1-(pyridin-4-ylmethyl)-N-(4-(1-(p-tolyl)pyrrolidin-2-yl)thiazol-2-yl)-1H- |
| | | | pyrrole-2-carboxamide |
| A2 | 1 | 502 | (R)-N-(4-(1-(4-(oxetan-3-yloxy)phenyl)pyrrolidin-2-yl)thiazol-2-yl)-1- |
| | | | (pyridin-4-ylmethyl)-1H-pyrrole-2-carboxamide |
| A4 | 1 | 516 | 1-(pyridin-4-ylmethyl)-N-(4-((R)-1-(4-(((S)-tetrahydrofuran-3- |
| | | | yl)oxy)phenyl)pyrrolidin-2-yl)thiazol-2-yl)-1H-pyrrole-2-carboxamide |
| A3 | 1 | 516 | 1-(pyridin-4-ylmethyl)-N-(4-((R)-1-(4-(((R)-tetrahydrofuran-3- |
| | | | yl)oxy)phenyl)pyrrolidin-2-yl)thiazol-2-yl)-1H-pyrrole-2-carboxamide |
| A1 | 1 | 530 | (R)-1-(pyridin-4-ylmethyl)-N-(4-(1-(4-((tetrahydro-2H-pyran-4- |
| | | | yl)oxy)phenyl)pyrrolidin-2-yl)thiazol-2-yl)-1H-pyrrole-2-carboxamide |
| Ints for | 1 | 510 | tert-butyl (E)-2,2-dimethyl-4-(2-(2-(1-(pyridin-4-ylmethyl)-1H-pyrrole-2- |
| B14 & | | | carboxamido)thiazol-5-yl)vinyl)oxazolidine-3-carboxylate |
| B12 | | | |
| Int for | 1 | 524 | tert-butyl (E)-2,2,4-trimethyl-4-(2-(2-(1-(pyridin-4-ylmethyl)-1H-pyrrole-2- |
| B16 | | | carboxamido)thiazol-5-yl)vinyl)oxazolidine-3-carboxylate |
| B19 | 1 | 422 | (E)-N-(4-(2-(1-methyl-6-oxopiperidin-2-yl)vinyl)thiazol-2-yl)-1-(pyridin-4- |
| | | | ylmethyl)-1H-pyrrole-2-carboxamide |
| B15 | 1 | 410 | (E)-N-(4-(2-(3-methyl-2-oxooxazolidin-4-yl)vinyl)thiazol-2-yl)-1-(pyridin-4- |
| | | | ylmethyl)-1H-pyrrole-2-carboxamide |
| Ints for | 1 | 542 | tert-butyl (E)-2-(2-(2-(1-(pyridin-4-ylmethyl)-1H-pyrrole-2- |
| B8 & B7 | | | carboxamido)thiazol-4-yl)vinyl)-3,4-dihydroquinoline-1(2H)-carboxylate |
| Ints for | 1 | 496 | tert-butyl (E)-2-(2-(2-(1-(pyridin-4-ylmethyl)-1H-pyrrole-2- |
| B9, B10 | | | carboxamido)thiazol-4-yl)vinyl)morpholine-4-carboxylate |
| and B6 | | | |
| B18 | 1 | 472 | (E)-N-(4-(2-(4-phenylmorpholin-2-yl)vinyl)thiazol-2-yl)-1-(pyridin-4- |
| | | | ylmethyl)-1H-pyrrole-2-carboxamide |
| B2 and | 1 | 392 | (E)-N-(4-(2-(2-methyloxazol-4-yl)vinyl)thiazol-2-yl)-1-(pyridin-4-ylmethyl)- |
| int for E4 | | | 1H-pyrrole-2-carboxamide |
| B13 | 1 | 408 | (E)-N-(4-(2-(6-oxopiperidin-2-yl)vinyl)thiazol-2-yl)-1-(pyridin-4-ylmethyl)- |
| | | | 1H-pyrrole-2-carboxamide |
| Ints for | 1 | 494 | tert-butyl (R,E)-2-(2-(2-(1-(pyridin-4-ylmethyl)-1H-pyrrole-2- |
| B11, E16 | | | carboxamido)thiazol-4-yl)vinyl)piperidine-1-carboxylate |
| and E18 | | | |
| B5 | 1 | 378 | (E)-N-(4-(2-(isoxazol-3-yl)vinyl)thiazol-2-yl)-1-(pyridin-4-ylmethyl)-1H- |
| | | | pyrrole-2-carboxamide |
| B1 | 1 | 378 | (E)-N-(4-(2-(oxazol-4-yl)vinyl)thiazol-2-yl)-1-(pyridin-4-ylmethyl)-1H- |
| | | | pyrrole-2-carboxamide |
| B3 | 1 | 391 | (E)-N-(4-(2-(1-methyl-1H-imidazol-4-yl)vinyl)thiazol-2-yl)-1-(pyridin-4- |
| | | | ylmethyl)-1H-pyrrole-2-carboxamide |
| B4 | 1 | 391 | (E)-N-(4-(2-(1-methyl-1H-pyrazol-3-yl)vinyl)thiazol-2-yl)-1-(pyridin-4- |
| | | | ylmethyl)-1H-pyrrole-2-carboxamide |
| C49 | 2 | 454 | (R)-1-(3-cyanobenzyl)-N-(4-(1-phenylpyrrolidin-2-yl)thiazol-2-yl)-1H- |
| | | | pyrrole-2-carboxamide |
| C50 | 2 | 454 | (R)-1-(4-cyanobenzyl)-N-(4-(1-phenylpyrrolidin-2-yl)thiazol-2-yl)-1H- |
| | | | pyrrole-2-carboxamide |
| Int for | 1 | 405 | (R)-1-(pent-4-yn-1-yl)-N-(4-(1-phenylpyrrolidin-2-yl)thiazol-2-yl)-1H- |
| C27 and | | | pyrrole-2-carboxamide |
| C31 | | | |
| C30 and | 1 | 419 | (R)-1-(hex-5-yn-1-yl)-N-(4-(1-phenylpyrrolidin-2-yl)thiazol-2-yl)-1H-pyrrole- |
| int for | | | 2-carboxamide |
| C26 | | | |
| C62 and | 1 | 391 | (R)-1-(but-3-yn-1-yl)-N-(4-(1-phenylpyrrolidin-2-yl)thiazol-2-yl)-1H-pyrrole- |
| int for | | | 2-carboxamide |
| C29 | | | |
| C57 | 1 | 435 | (R)-1-((3,6-dihydro-2H-pyran-4-yl)methyl)-N-(4-(1-phenylpyrrolidin-2- |
| | | | yl)thiazol-2-yl)-1H-pyrrole-2-carboxamide |
| C21 | 1 | 471 | (R)-1-((4,4-difluorocyclohexyl)methyl)-N-(4-(1-phenylpyrrolidin-2- |
| | | | yl)thiazol-2-yl)-1H-pyrrole-2-carboxamide |
| C37 | 1 | 485 | (R)-1-((1,1-dioxidotetrahydro-2H-thiopyran-4-yl)methyl)-N-(4-(1- |
| | | | phenylpyrrolidin-2-yl)thiazol-2-yl)-1H-pyrrole-2-carboxamide |
| C23 | 1 | 425 | (R)-1-(4-methoxybutyl)-N-(4-(1-phenylpyrrolidin-2-yl)thiazol-2-yl)-1H- |
| | | | pyrrole-2-carboxamide |
| Int for | 1 | 377 | (R)-N-(4-(1-phenylpyrrolidin-2-yl)thiazol-2-yl)-1-(prop-2-yn-1-yl)-1H- |
| C29 and | | | pyrrole-2-carboxamide |
| C33 | | | |
| C39 | 1 | 435 | (R)-1-(cyclohexylmethyl)-N-(4-(1-phenylpyrrolidin-2-yl)thiazol-2-yl)-1H- |
| | | | pyrrole-2-carboxamide |
| C3 | 2 | 437 | N-(4-((R)-1-phenylpyrrolidin-2-yl)thiazol-2-yl)-1-(2-(tetrahydrofuran-3- |
| | | | yl)ethyl)-1H-pyrrole-2-carboxamide |
| C18 | 1 | 446 | 1-(((1R,2S)-2-(cyanomethyl)cyclobutyl)methyl)-N-(4-((R)-1- |
| | | | phenylpyrrolidin-2-yl)thiazol-2-yl)-1H-pyrrole-2-carboxamide |
| C19 | 1 | 448 | (R)-1-((3-(cyanomethyl)oxetan-3-yl)methyl)-N-(4-(1-phenylpyrrolidin-2- |
| | | | yl)thiazol-2-yl)-1H-pyrrole-2-carboxamide |
| C20 | 1 | 420 | 1-(3-cyano-2-methylpropyl)-N-(4-((R)-1-phenylpyrrolidin-2-yl)thiazol-2-yl)- |
| | | | 1H-pyrrole-2-carboxamide |
| Ints for | 1 | 495 | tert-butyl (R)-5-(2-((4-(1-phenylpyrrolidin-2-yl)thiazol-2-yl)carbamoyl)-1H- |
| C11 and | | | pyrrol-1-yl)pentanoate |
| C60 | | | |
| Ints for | 1 | 467 | tert-butyl (R)-3-(2-((4-(1-phenylpyrrolidin-2-yl)thiazol-2-yl)carbamoyl)-1H- |
| C12 and | | | pyrrol-1-yl)propanoate |
| C59 | | | |
| C6 | 1 | 418 | 1-((2-cyanocyclopropyl)methyl)-N-(4-((R)-1-phenylpyrrolidin-2-yl)thiazol-2- |
| | | | yl)-1H-pyrrole-2-carboxamide |
| C9 | 1 | 446 | (R)-1-((1-(cyanomethyl)cyclobutyl)methyl)-N-(4-(1-phenylpyrrolidin-2- |
| | | | yl)thiazol-2-yl)-1H-pyrrole-2-carboxamide |
| C53 | 2 | 451 | (R)-1-((tetrahydro-2H-pyran-4-yl)methyl)-N-(4-(1-(p-tolyl)pyrrolidin-2- |
| | | | yl)thiazol-2-yl)-1H-pyrrole-2-carboxamide |
| Ints for | 1 | 481 | tert-butyl (R)-4-(2-((4-(1-phenylpyrrolidin-2-yl)thiazol-2-yl)carbamoyl)-1H- |
| C13 and | | | pyrrol-1-yl)butanoate |
| C61 | | | |
| C54 | 2 | 521 | (R)-1-((tetrahydro-2H-pyran-4-yl)methyl)-N-(4-(1-(4- |
| | | | (trifluoromethoxy)phenyl)pyrrolidin-2-yl)thiazol-2-yl)-1H-pyrrole-2- |
| | | | carboxamide |
| C1 and | 1 | 420 | (R)-1-(4-cyanobutyl)-N-(4-(1-phenylpyrrolidin-2-yl)thiazol-2-yl)-1H-pyrrole- |
| ints for | | | 2-carboxamide |
| C4& C58 | | | |
| C2 and | 1 | 406 | (R)-1-(3-cyanopropyl)-N-(4-(1-phenylpyrrolidin-2-yl)thiazol-2-yl)-1H- |
| Ints for | | | pyrrole-2-carboxamide |
| C14 &C7 | | | |
| C32 and | 1 | 392 | (R)-1-(2-cyanoethyl)-N-(4-(1-phenylpyrrolidin-2-yl)thiazol-2-yl)-1H-pyrrole- |
| Ints for | | | 2-carboxamide |
| C16 & | | | |
| C15 | | | |
| Int for | 1 | 460 | (R)-1-((6-methoxypyridin-3-yl)methyl)-N-(4-(1-phenylpyrrolidin-2- |
| C10 | | | yl)thiazol-2-yl)-1H-pyrrole-2-carboxamide |
| C8 | 1 | 432 | (R)-1-((1-(cyanomethyl)cyclopropyl)methyl)-N-(4-(1-phenylpyrrolidin-2- |
| | | | yl)thiazol-2-yl)-1H-pyrrole-2-carboxamide |
| C55 | 2 | 437 | (R)-N-(4-(1-phenylpyrrolidin-2-yl)thiazol-2-yl)-1-((tetrahydro-2H-pyran-4- |
| | | | yl)methyl)-1H-pyrrole-2-carboxamide |
| C5 | 2 | 429 | (R)-1-benzyl-N-(4-(1-phenylpyrrolidin-2-yl)thiazol-2-yl)-1H-pyrrole-2- |
| | | | carboxamide |
| C17 | 2 | 353 | (R)-1-methyl-N-(4-(1-phenylpyrrolidin-2-yl)thiazol-2-yl)-1H-pyrrole-2- |
| | | | carboxamide |
| Int for | 1 | 454 | tert-butyl (E)-methyl(3-(2-(1-(pyridin-4-ylmethyl)-1H-pyrrole-2- |
| E15 | | | carboxamido)thiazol-4-yl)allyl)carbamate |
| Ints for | 1 | 440 | tert-butyl (E)-(3-(2-(1-(pyridin-4-ylmethyl)-1H-pyrrole-2- |
| E30 and | | | carboxamido)thiazol-4-yl)allyl)carbamate |
| E54 | | | |
| E12 | 1 | 456 | N-(4-(2-(2-phenyloxazol-4-yl)ethyl)thiazol-2-yl)-1-(pyridin-4-ylmethyl)-1H- |
| | | | pyrrole-2-carboxamide |
| E2 | 1 | 486 | (E)-1-(pyridin-4-ylmethyl)-N-(4-(1-(5-(trifluoromethoxy)pyridin-2-yl)prop-1- |
| | | | en-2-yl)thiazol-2-yl)-1H-pyrrole-2-carboxamide |
| E1 | 1 | 402 | (E)-N-(4-(2-(4-methylpyridin-2-yl)vinyl)thiazol-2-yl)-1-(pyridin-4-ylmethyl)- |
| | | | 1H-pyrrole-2-carboxamide |
| E11 | 1 | 376 | N-(4-(pyridin-2-ylmethyl)thiazol-2-yl)-1-(pyridin-4-ylmethyl)-1H-pyrrole-2- |
| | | | carboxamide |
| E5 | 1 | 486 | (E)-1-(pyridin-4-ylmethyl)-N-(4-(2-(5-(trifluoromethoxy)pyridin-2-yl)prop-1- |
| | | | en-1-yl)thiazol-2-yl)-1H-pyrrole-2-carboxamide |
| E9 | 1 | 383 | 1-(pyridin-4-ylmethyl)-N-(4-((tetrahydro-2H-pyran-2-yl)methyl)thiazol-2-yl)- |
| | | | 1H-pyrrole-2-carboxamide |
| E8 | 1 | 390 | N-(4-(2-(pyridin-2-yl)ethyl)thiazol-2-yl)-1-(pyridin-4-ylmethyl)-1H-pyrrole-2- |
| | | | carboxamide |
| E6 | 1 | 397 | 1-(pyridin-4-ylmethyl)-N-(4-(2-(tetrahydro-2H-pyran-2-yl)ethyl)thiazol-2-yl)- |
| | | | 1H-pyrrole-2-carboxamide |
| E3 | 1 | 386 | N-(4-(pyridin-2-ylethynyl)thiazol-2-yl)-1-(pyridin-4-ylmethyl)-1H-pyrrole-2- |
| | | | carboxamide |
| E7 | 1 | 474 | 1-(pyridin-4-ylmethyl)-N-(4-(2-(5-(trifluoromethoxy)pyridin-2- |
| | | | yl)ethyl)thiazol-2-yl)-1H-pyrrole-2-carboxamide |
| E46 | 1 | 431 | (R)-N-(4-(1-(pyridin-3-yl)pyrrolidin-2-yl)thiazol-2-yl)-1-(pyridin-4-ylmethyl)- |
| | | | 1H-pyrrole-2-carboxamide |
| E33 | 1 | 443 | N-(4-(3-(pyridin-2-yl)-3-azabicyclo[3.1.0]hexan-6-yl)thiazol-2-yl)-1- |
| | | | (pyridin-4-ylmethyl)-1H-pyrrole-2-carboxamide |
| E36 | 1 | 442 | N-(4-(3-phenyl-3-azabicyclo[3.1.0]hexan-6-yl)thiazol-2-yl)-1-(pyridin-4- |
| | | | ylmethyl)-1H-pyrrole-2-carboxamide |
| E47 | 1 | 445 | (R)-N-(4-(1-(pyridin-2-yl)piperidin-3-yl)thiazol-2-yl)-1-(pyridin-4-ylmethyl)- |
| | | | 1H-pyrrole-2-carboxamide |
| E45 | 1 | 445 | (S)-N-(4-(1-(pyridin-2-yl)piperidin-3-yl)thiazol-2-yl)-1-(pyridin-4-ylmethyl)- |
| | | | 1H-pyrrole-2-carboxamide |
| E43 | 1 | 431 | (R)-N-(4-(1-(pyridin-2-yl)pyrrolidin-3-yl)thiazol-2-yl)-1-(pyridin-4-ylmethyl)- |
| | | | 1H-pyrrole-2-carboxamide |
| E44 | 1 | 431 | (S)-N-(4-(1-(pyridin-2-yl)pyrrolidin-3-yl)thiazol-2-yl)-1-(pyridin-4-ylmethyl)- |
| | | | 1H-pyrrole-2-carboxamide |
| A29 | 1 | 432 | (R)-N-(4-(1-(pyridazin-3-yl)pyrrolidin-2-yl)thiazol-2-yl)-1-(pyridin-4- |
| | | | ylmethyl)-1H-pyrrole-2-carboxamide |
| Ints for | 1 | 604 | N-(4-((2R,4R)-4-(2-((tert-butyldimethylsilyl)oxy)ethoxy)-1-phenylpyrrolidin- |
| E31 and | | | 2-yl)thiazol-2-yl)-1-(pyridin-4-ylmethyl)-1H-pyrrole-2-carboxamide |
| E32 | | | |
| E40 | 1 | 571 | N-(4-((2R,4R)-4-((1-acetylpiperidin-4-yl)oxy)-1-phenylpyrrolidin-2- |
| | | | yl)thiazol-2-yl)-1-(pyridin-4-ylmethyl)-1H-pyrrole-2-carboxamide |
| E13 | 1 | 444 | N-(4-((R)-1-phenylpyrrolidin-2-yl)thiazol-2-yl)-1-(1-(pyridin-4-yl)ethyl)-1H- |
| | | | pyrrole-2-carboxamide |
| E37 | 1 | 402 | N-(4-(6,7-dihydro-5H-cyclopenta[b]pyridin-6-yl)thiazol-2-yl)-1-(pyridin-4- |
| | | | ylmethyl)-1H-pyrrole-2-carboxamide |
| E35 | 1 | 490 | N-(4-((1s,4s)-4-((5-methoxypyridin-2-yl)oxy)cyclohexyl)thiazol-2-yl)-1- |
| | | | (pyridin-4-ylmethyl)-1H-pyrrole-2-carboxamide |
| E39 | 1 | 490 | N-(4-((1S,3R)-3-((5-methoxypyridin-2-yl)oxy)cyclohexyl)thiazol-2-yl)-1- |
| | | | (pyridin-4-ylmethyl)-1H-pyrrole-2-carboxamide |
| E41 | 1 | 490 | N-(4-((1S,3S)-3-((5-methoxypyridin-2-yl)oxy)cyclohexyl)thiazol-2-yl)-1- |
| | | | (pyridin-4-ylmethyl)-1H-pyrrole-2-carboxamide |
| E38 | 1 | 476 | N-(4-((1S,3S)-3-((5-methoxypyridin-2-yl)oxy)cyclopentyl)thiazol-2-yl)-1- |
| | | | (pyridin-4-ylmethyl)-1H-pyrrole-2-carboxamide |
| E34 | 1 | 490 | N-(4-((1r,4r)-4-((5-methoxypyridin-2-yl)oxy)cyclohexyl)thiazol-2-yl)-1- |
| | | | (pyridin-4-ylmethyl)-1H-pyrrole-2-carboxamide |
| E42 | 1 | 476 | N-(4-((1S,3R)-3-((5-methoxypyridin-2-yl)oxy)cyclopentyl)thiazol-2-yl)-1- |
| | | | (pyridin-4-ylmethyl)-1H-pyrrole-2-carboxamide |
| E10 | 1 | 458 | (R)-N-(4-(1-phenylpyrrolidin-2-yl)thiazol-2-yl)-1-(3-(pyridin-3-yl)propyl)-1H- |
| | | | pyrrole-2-carboxamide |
| E26 and | 2 | 508 | (R)-1-((2-bromopyridin-4-yl)methyl)-N-(4-(1-phenylpyrrolidin-2-yl)thiazol-2- |
| int for | | | yl)-1H-pyrrole-2-carboxamide |
| E60 | | | |
| E22 and | 2 | 508 | (R)-1-((3-bromopyridin-4-yl)methyl)-N-(4-(1-phenylpyrrolidin-2-yl)thiazol-2- |
| ints for | | | yl)-1H-pyrrole-2-carboxamide |
| E59&E58 | | | |
| E57 | 1 | 430 | (E)-N-(4-(2-(5-isopropylpyridin-2-yl)vinyl)thiazol-2-yl)-1-(pyridin-4- |
| | | | ylmethyl)-1H-pyrrole-2-carboxamide |
| E56 | 1 | 430 | (E)-N-(4-(2-(3-isopropylpyridin-2-yl)vinyl)thiazol-2-yl)-1-(pyridin-4- |
| | | | ylmethyl)-1H-pyrrole-2-carboxamide |
| E55 | 1 | 430 | (E)-N-(4-(2-(6-isopropylpyridin-2-yl)vinyl)thiazol-2-yl)-1-(pyridin-4- |
| | | | ylmethyl)-1H-pyrrole-2-carboxamide |
| B20 | 1 | 391 | (E)-N-(4-(2-(1-methyl-1H-imidazol-2-yl)vinyl)thiazol-2-yl)-1-(pyridin-4- |
| | | | ylmethyl)-1H-pyrrole-2-carboxamide |
| E26 | 1 | 448 | (R)-1-((2-fluoropyridin-4-yl)methyl)-N-(4-(1-phenylpyrrolidin-2-yl)thiazol-2- |
| | | | yl)-1H-pyrrole-2-carboxamide |
| E27 | 1 | 448 | (R)-1-((3-fluoropyridin-4-yl)methyl)-N-(4-(1-phenylpyrrolidin-2-yl)thiazol-2- |
| | | | yl)-1H-pyrrole-2-carboxamide |
| E20 | 1 | 430 | (R)-N-(4-(1-phenylpyrrolidin-2-yl)thiazol-2-yl)-1-(pyridin-3-ylmethyl)-1H- |
| | | | pyrrole-2-carboxamide |
| E21 | 1 | 444 | (R)-N-(4-(1-phenylpyrrolidin-2-yl)thiazol-2-yl)-1-(2-(pyridin-3-yl)ethyl)-1H- |
| | | | pyrrole-2-carboxamide |
| Common | 1 | 454 | tert-butyl (R)-2-(2-(1-(pyridin-4-ylmethyl)-1H-pyrrole-2- |
| intermediate | | | carboxamido)thiazol-4-yl)pyrrolidine-1-carboxylate |
| B33 | 1 | 392 | (E)-N-(4-(2-(5-methyloxazol-4-yl)vinyl)thiazol-2-yl)-1-(pyridin-4-ylmethyl)- |
| | | | 1H-pyrrole-2-carboxamide |
| B34 | 1 | 378 | (E)-N-(4-(2-(oxazol-2-yl)vinyl)thiazol-2-yl)-1-(pyridin-4-ylmethyl)-1H- |
| | | | pyrrole-2-carboxamide |
| B35 | 1 | 392 | (E)-N-(4-(2-(5-methyloxazol-2-yl)vinyl)thiazol-2-yl)-1-(pyridin-4-ylmethyl)- |
| | | | 1H-pyrrole-2-carboxamide |
| B36 | 1 | 434 | (E)-N-(4-(2-(2-(tert-butyl)oxazol-4-yl)vinyl)thiazol-2-yl)-1-(pyridin-4- |
| | | | ylmethyl)-1H-pyrrole-2-carboxamide |
| B37 | 1 | 419 | (E)-N-(4-(2-(1-isopropyl-1H-imidazol-4-yl)vinyl)thiazol-2-yl)-1-(pyridin-4- |
| | | | ylmethyl)-1H-pyrrole-2-carboxamide |
| C84 | 2 | 409 | (R)-1-(oxetan-3-ylmethyl)-N-(4-(1-phenylpyrrolidin-2-yl)thiazol-2-yl)-1H- |
| | | | pyrrole-2-carboxamide |
| B38 | 1 | 420 | (E)-N-(4-(2-(2-isopropyloxazol-4-yl)vinyl)thiazol-2-yl)-1-(pyridin-4- |
| | | | ylmethyl)-1H-pyrrole-2-carboxamide |
| B39 | 1 | 460 | (E)-N-(4-(2-(2-cyclohexyloxazol-4-yl)vinyl)thiazol-2-yl)-1-(pyridin-4- |
| | | | ylmethyl)-1H-pyrrole-2-carboxamide |
| B40 | 1 | 418 | (E)-N-(4-(2-(2-cyclopropyloxazol-4-yl)vinyl)thiazol-2-yl)-1-(pyridin-4- |
| | | | ylmethyl)-1H-pyrrole-2-carboxamide |
| E63 | 1 | 445 | (R)-N-(4-(1-(4-methylpyridin-2-yl)pyrrolidin-2-yl)thiazol-2-yl)-1-(pyridin-4- |
| | | | ylmethyl)-1H-pyrrole-2-carboxamide |
| E62 | 1 | 445 | (R)-N-(4-(1-(5-methylpyridin-2-yl)pyrrolidin-2-yl)thiazol-2-yl)-1-(pyridin-4- |
| | | | ylmethyl)-1H-pyrrole-2-carboxamide |
| B41 | 1 | 392 | (E)-N-(4-(2-(4-methyloxazol-2-yl)vinyl)thiazol-2-yl)-1-(pyridin-4-ylmethyl)- |
| | | | 1H-pyrrole-2-carboxamide |
| Int. for | 1 | 559 | tert-butyl (R)-(4-(2-(2-(1-(pyridin-4-ylmethyl)-1H-pyrrole-2- |
| A44 and | | | carboxamido)thiazol-4-yl)pyrrolidin-1-yl)benzyl)carbamate |
| A45 | | | |
| Int. for | 1 | 545 | tert-butyl (R)-(4-(2-(2-(1-(pyridin-4-ylmethyl)-1H-pyrrole-2- |
| A46&A47 | | | carboxamido)thiazol-4-yl)pyrrolidin-1-yl)phenyl)carbamate |
| C85 | 1 | 466 | (R)-N-(4-(1-(5-ethylpyridin-2-yl)pyrrolidin-2-yl)thiazol-2-yl)-1-((tetrahydro- |
| | | | 2H-pyran-4-yl)methyl)-1H-pyrrole-2-carboxamide |
| A48 | 1 | 474 | (R)-N-(4-(1-(4-(methoxymethyl)phenyl)pyrrolidin-2-yl)thiazol-2-yl)-1- |
| | | | (pyridin-4-ylmethyl)-1H-pyrrole-2-carboxamide |
| Int. for | 1 | 698 | (R)-N-(4-(1-(4-(((tert-butyldiphenylsilyl)oxy)methyl)phenyl)pyrrolidin-2- |
| A57 | | | yl)thiazol-2-yl)-1-(pyridin-4-ylmethyl)-1H-pyrrole-2-carboxamide |
| Int. for | 1 | 573 | tert-butyl (R)-methyl(4-(2-(2-(1-(pyridin-4-ylmethyl)-1H-pyrrole-2- |
| A49 | | | carboxamido)thiazol-4-yl)pyrrolidin-1-yl)benzyl)carbamate |
| E65 | 1 | 458 | (R)-N-(4-(1-(5-ethylpyridin-2-yl)pyrrolidin-2-yl)thiazol-2-yl)-1-(pyridin-4- |
| | | | ylmethyl)-1H-pyrrole-2-carboxamide |
| C86 | 1 | 454 | (R)-1-(2-cyanobenzyl)-N-(4-(1-phenylpyrrolidin-2-yl)thiazol-2-yl)-1H- |
| | | | pyrrole-2-carboxamide |
| C87 | 1 | 420 | 1-((S)-3-cyano-2-methylpropyl)-N-(4-((R)-1-phenylpyrrolidin-2-yl)thiazol-2- |
| | | | yl)-1H-pyrrole-2-carboxamide |
| Int. for | 1 | 613 | tert-butyl (R)-4-(4-(2-(2-(1-(pyridin-4-ylmethyl)-1H-pyrrole-2- |
| A51&A55 | | | carboxamido)thiazol-4-yl)pyrrolidin-1-yl)phenyl)piperidine-1-carboxylate |
| A52 | 1 | 444 | (R)-1-((2-methylpyridin-4-yl)methyl)-N-(4-(1-phenylpyrrolidin-2-yl)thiazol- |
| | | | 2-yl)-1H-pyrrole-2-carboxamide |
| A53 | 1 | 444 | (R)-1-((3-methylpyridin-4-yl)methyl)-N-(4-(1-phenylpyrrolidin-2-yl)thiazol- |
| | | | 2-yl)-1H-pyrrole-2-carboxamide |
| Int. for | 1 | 619 | (E)-1-(pyridin-4-ylmethyl)-N-(4-(2-(1-trityl-1H-imidazol-4-yl)vinyl)thiazol-2- |
| B42 | | | yl)-1H-pyrrole-2-carboxamide |
| E64 | 1 | 445 | (R)-N-(4-(1-(3-methylpyridin-2-yl)pyrrolidin-2-yl)thiazol-2-yl)-1-(pyridin-4- |
| | | | ylmethyl)-1H-pyrrole-2-carboxamide |
| C88 | 1 | 420 | 1-((R)-3-cyano-2-methylpropyl)-N-(4-((R)-1-phenylpyrrolidin-2-yl)thiazol-2- |
| | | | yl)-1H-pyrrole-2-carboxamide |
| C89 | 1 | 420 | 1-(3-cyanobutyl)-N-(4-((R)-1-phenylpyrrolidin-2-yl)thiazol-2-yl)-1H-pyrrole- |
| | | | 2-carboxamide |
| C90 | 1 | 420 | 1-(4-cyanobutan-2-yl)-N-(4-((R)-1-phenylpyrrolidin-2-yl)thiazol-2-yl)-1H- |
| | | | pyrrole-2-carboxamide |
| C91 | 2 | 505 | (R)-1-((tetrahydro-2H-pyran-4-yl)methyl)-N-(4-(1-(4- |
| | | | (trifluoromethyl)phenyl)pyrrolidin-2-yl)thiazol-2-yl)-1H-pyrrole-2- |
| | | | carboxamide |
| A56 | 2 | 516 | (R)-1-((2-fluoropyridin-4-yl)methyl)-N-(4-(1-(4- |
| | | | (trifluoromethyl)phenyl)pyrrolidin-2-yl)thiazol-2-yl)-1H-pyrrole-2- |
| | | | carboxamide |
| C92 | 2 | 451 | N-(4-((R)-1-phenylpyrrolidin-2-yl)thiazol-2-yl)-1-(3-(tetrahydrofuran-3- |
| | | | yl)propyl)-1H-pyrrole-2-carboxamide |
| C93 | 1 | 468 | (R)-1-(2-(cyanomethyl)benzyl)-N-(4-(1-phenylpyrrolidin-2-yl)thiazol-2-yl)- |
| | | | 1H-pyrrole-2-carboxamide |
| D2 | 1 | 431 | (E)-N-(4-(2-(3,5-difluoropyridin-2-yl)vinyl)thiazol-2-yl)-1-((tetrahydro-2H- |
| | | | pyran-4-yl)methyl)-1H-pyrrole-2-carboxamide |
| C94 | 1 | 443 | (R)-1-((3,3-difluorocyclobutyl)methyl)-N-(4-(1-phenylpyrrolidin-2-yl)thiazol- |
| | | | 2-yl)-1H-pyrrole-2-carboxamide |
| C95 | 1 | 459 | (R)-N-(4-(1-(6-ethylpyridin-3-yl)pyrrolidin-2-yl)thiazol-2-yl)-1-(pyridin-4- |
| | | | ylmethyl)-1H-pyrrole-2-carboxamide |
| C96 | 1 | 466 | (R)-N-(4-(1-(6-ethylpyridin-3-yl)pyrrolidin-2-yl)thiazol-2-yl)-1-((tetrahydro- |
| | | | 2H-pyran-4-yl)methyl)-1H-pyrrole-2-carboxamide |
| C97 | 1 | 436 | 1-((S)-3-cyano-2-methoxypropyl)-N-(4-((R)-1-phenylpyrrolidin-2-yl)thiazol- |
| | | | 2-yl)-1H-pyrrole-2-carboxamide |
| Int. for | 1 | 613 | tert-butyl 2-(4-((R)-2-(2-(1-(pyridin-4-ylmethyl)-1H-pyrrole-2- |
| A58 and | | | carboxamido)thiazol-4-yl)pyrrolidin-1-yl)phenyl)piperidine-1-carboxylate |
| A59 | | | |
| B43 | 1 | 389 | (E)-N-(4-(2-(pyrazin-2-yl)vinyl)thiazol-2-yl)-1-(pyridin-4-ylmethyl)-1H- |
| | | | pyrrole-2-carboxamide |
| C99 | 2 | 423 | (R)-1-(2-(oxetan-3-yl)ethyl)-N-(4-(1-phenylpyrrolidin-2-yl)thiazol-2-yl)-1H- |
| | | | pyrrole-2-carboxamide |
| Int. for | 1 | 506 | 1-((2S)-3-cyano-2-((tetrahydro-2H-pyran-2-yl)oxy)propyl)-N-(4-((R)-1- |
| C100 | | | phenylpyrrolidin-2-yl)thiazol-2-yl)-1H-pyrrole-2-carboxamide |
| A60 | 1 | 466 | (R)-1-((2,6-difluoropyridin-4-yl)methyl)-N-(4-(1-phenylpyrrolidin-2- |
| | | | yl)thiazol-2-yl)-1H-pyrrole-2-carboxamide |
| C102 | 1 | 431 | (R)-N-(4-(1-phenylpyrrolidin-2-yl)thiazol-2-yl)-1-(pyridazin-4-ylmethyl)-1H- |
| | | | pyrrole-2-carboxamide |
| B44 | 1 | 389 | (E)-1-(pyridin-4-ylmethyl)-N-(4-(2-(pyrimidin-4-yl)vinyl)thiazol-2-yl)-1H- |
| | | | pyrrole-2-carboxamide |
| Int. for | 1 | 613 | tert-butyl (R)-3-(4-((R)-2-(2-(1-(pyridin-4-ylmethyl)-1H-pyrrole-2- |
| A61 and | | | carboxamido)thiazol-4-yl)pyrrolidin-1-yl)phenyl)piperidine-1-carboxylate |
| A63 | | | |
| Int. for | 2 | 636 | tert-butyl (R)-4-(4-(2-(2-(1-((tetrahydro-2H-pyran-4-yl)methyl)-1H-pyrrole- |
| C103 | | | 2-carboxamido)thiazol-4-yl)pyrrolidin-1-yl)phenoxy)piperidine-1- |
| | | | carboxylate |
| common | 1 | 647 | tert-butyl (R)-4-(4-(2-(2-(1-((2-fluoropyridin-4-yl)methyl)-1H-pyrrole-2- |
| intermediate | | | carboxamido)thiazol-4-yl)pyrrolidin-1-yl)phenoxy)piperidine-1-carboxylate |
| Int. for | 1 | 577 | tert-butyl (R)-(4-(2-(2-(1-((2-fluoropyridin-4-yl)methyl)-1H-pyrrole-2- |
| A64 | | | carboxamido)thiazol-4-yl)pyrrolidin-1-yl)benzyl)carbamate |
| Int. for | 1 | 566 | tert-butyl (R)-(4-(2-(2-(1-((tetrahydro-2H-pyran-4-yl)methyl)-1H-pyrrole-2- |
| C104 | | | carboxamido)thiazol-4-yl)pyrrolidin-1-yl)benzyl)carbamate |
| Int. for | 1 | 473 | (R)-N-(4-(1-(4-cyanophenyl)pyrrolidin-2-yl)thiazol-2-yl)-1-((2-fluoropyridin- |
| A65 | | | 4-yl)methyl)-1H-pyrrole-2-carboxamide |
| Int. for | 1 | 462 | (R)-N-(4-(1-(4-cyanophenyl)pyrrolidin-2-yl)thiazol-2-yl)-1-((tetrahydro-2H- |
| C104 | | | pyran-4-yl)methyl)-1H-pyrrole-2-carboxamide |
| Int. for | 1 | 573 | tert-butyl ((R)-1-(4-((R)-2-(2-(1-(pyridin-4-ylmethyl)-1H-pyrrole-2- |
| A66 and | | | carboxamido)thiazol-4-yl)pyrrolidin-1-yl)phenyl)ethyl)carbamate |
| A67 | | | |
| C106 | 2 | 432 | (R)-1-((3-cyanocyclobutyl)methyl)-N-(4-(1-phenylpyrrolidin-2-yl)thiazol-2- |
| | | | yl)-1H-pyrrole-2-carboxamide |
| B45 | 1 | 437 | (E)-1-((2-fluoropyridin-4-yl)methyl)-N-(4-(2-(1-isopropyl-1H-imidazol-4- |
| | | | yl)vinyl)thiazol-2-yl)-1H-pyrrole-2-carboxamide |
| B47 | 1 | 410 | (E)-1-((2-fluoropyridin-4-yl)methyl)-N-(4-(2-(5-methyloxazol-4- |
| | | | yl)vinyl)thiazol-2-yl)-1H-pyrrole-2-carboxamide |
| common | 1 | 563 | tert-butyl (R)-(4-(2-(2-(1-((2-fluoropyridin-4-yl)methyl)-1H-pyrrole-2- |
| intermediate | | | carboxamido)thiazol-4-yl)pyrrolidin-1-yl)phenyl)carbamate |
| Int. for | 1 | 552 | tert-butyl (R)-(4-(2-(2-(1-((tetrahydro-2H-pyran-4-yl)methyl)-1H-pyrrole-2- |
| C107 | | | carboxamido)thiazol-4-yl)pyrrolidin-1-yl)phenyl)carbamate |
| Int. for | 1 | 591 | tert-butyl ((S)-1-(4-((R)-2-(2-(1-((2-fluoropyridin-4-yl)methyl)-1H-pyrrole-2- |
| A69 and | | | carboxamido)thiazol-4-yl)pyrrolidin-1-yl)phenyl)ethyl)carbamate |
| A74 | | | |
| Int. for | 1 | 591 | tert-butyl ((R)-1-(4-((R)-2-(2-(1-((2-fluoropyridin-4-yl)methyl)-1H-pyrrole-2- |
| A70 and | | | carboxamido)thiazol-4-yl)pyrrolidin-1-yl)phenyl)ethyl)carbamate |
| A72 | | | |
| Int. for | 1 | 573 | tert-butyl ((S)-1-(4-((R)-2-(2-(1-(pyridin-4-ylmethyl)-1H-pyrrole-2- |
| A71 and | | | carboxamido)thiazol-4-yl)pyrrolidin-1-yl)phenyl)ethyl)carbamate |
| A73 | | | |
| B48 | 1 | 420 | (E)-N-(4-(2-(6-fluoro-5-methylpyridin-2-yl)vinyl)thiazol-2-yl)-1-(pyridin-4- |
| | | | ylmethyl)-1H-pyrrole-2-carboxamide |
| B49 | 1 | 438 | (E)-N-(4-(2-(6-fluoro-5-methylpyridin-2-yl)vinyl)thiazol-2-yl)-1-((2- |
| | | | fluoropyridin-4-yl)methyl)-1H-pyrrole-2-carboxamide |
| D3 | 1 | 427 | (E)-N-(4-(2-(6-fluoro-5-methylpyridin-2-yl)vinyl)thiazol-2-yl)-1-((tetrahydro- |
| | | | 2H-pyran-4-yl)methyl)-1H-pyrrole-2-carboxamide |
| B50 | 1 | 427 | (E)-N-(4-(2-(imidazo[1,2-a]pyridin-2-yl)vinyl)thiazol-2-yl)-1-(pyridin-4- |
| | | | ylmethyl)-1H-pyrrole-2-carboxamide |
| A75 | 1 | 499 | (R)-N-(4-(1-(2-oxo-1,2,3,4-tetrahydroquinolin-6-yl)pyrrolidin-2-yl)thiazol-2- |
| | | | yl)-1-(pyridin-4-ylmethyl)-1H-pyrrole-2-carboxamide |
| C108 | 1 | 436 | 1-((R)-3-cyano-2-methoxypropyl)-N-(4-((R)-1-phenylpyrrolidin-2-yl)thiazol- |
| | | | 2-yl)-1H-pyrrole-2-carboxamide |
| B51 | 1 | 405 | (E)-N-(4-(2-(1,2-dimethyl-1H-imidazol-4-yl)vinyl)thiazol-2-yl)-1-(pyridin-4- |
| | | | ylmethyl)-1H-pyrrole-2-carboxamide |
| | | | Coupling done in DMF* |
| B52 | 1 | 460 | (E)-N-(4-(2-(5-cyclohexyloxazol-4-yl)vinyl)thiazol-2-yl)-1-(pyridin-4- |
| | | | ylmethyl)-1H-pyrrole-2-carboxamide |
| B53 | 1 | 478 | (E)-N-(4-(2-(5-cyclohexyloxazol-4-yl)vinyl)thiazol-2-yl)-1-((2-fluoropyridin- |
| | | | 4-yl)methyl)-1H-pyrrole-2-carboxamide |
| D4 | 1 | 467 | (E)-N-(4-(2-(5-cyclohexyloxazol-4-yl)vinyl)thiazol-2-yl)-1-((tetrahydro-2H- |
| | | | pyran-4-yl)methyl)-1H-pyrrole-2-carboxamide |
| A76 | 1 | 404 | N-(4-(3-(dimethylamino)phenyl)thiazol-2-yl)-1-(pyridin-4-ylmethyl)-1H- |
| | | | pyrrole-2-carboxamide |
| A77 | 2 | 476 | (R)-N-(4-(1-(4-ethylphenyl)pyrrolidin-2-yl)thiazol-2-yl)-1-((2-fluoropyridin- |
| | | | 4-yl)methyl)-1H-pyrrole-2-carboxamide |
| C110 | 2 | 465 | (R)-N-(4-(1-(4-ethylphenyl)pyrrolidin-2-yl)thiazol-2-yl)-1-((tetrahydro-2H- |
| | | | pyran-4-yl)methyl)-1H-pyrrole-2-carboxamide |
| B54 | 1 | 459 | (E)-N-(4-(2-(1-cyclohexyl-1H-imidazol-4-yl)vinyl)thiazol-2-yl)-1-(pyridin-4- |
| | | | ylmethyl)-1H-pyrrole-2-carboxamide |
| B55 | 1 | 477 | (E)-N-(4-(2-(1-cyclohexyl-1H-imidazol-4-yl)vinyl)thiazol-2-yl)-1-((2- |
| | | | fluoropyridin-4-yl)methyl)-1H-pyrrole-2-carboxamide |
| D5 | 1 | 466 | (E)-N-(4-(2-(1-cyclohexyl-1H-imidazol-4-yl)vinyl)thiazol-2-yl)-1- |
| | | | ((tetrahydro-2H-pyran-4-yl)methyl)-1H-pyrrole-2-carboxamide |
| B56 and | 1 | 435 | (E)-N-(4-(2-(1-(2-methoxyethyl)-1H-imidazol-4-yl)vinyl)thiazol-2-yl)-1- |
| Int. for | | | (pyridin-4-ylmethyl)-1H-pyrrole-2-carboxamide |
| C114 | | | |
| C111 | 1 | 466 | (R)-1-(isoquinolin-5-yl)-N-(4-(1-phenylpyrrolidin-2-yl)thiazol-2-yl)-1H- |
| | | | pyrrole-2-carboxamide |
| Int. for | 1 | 506 | 1-((2R)-3-cyano-2-((tetrahydro-2H-pyran-2-yl)oxy)propyl)-N-(4-((R)-1- |
| C112 | | | phenylpyrrolidin-2-yl)thiazol-2-yl)-1H-pyrrole-2-carboxamide |
| B57 | 1 | 419 | (E)-N-(4-(2-(1-isopropyl-1H-imidazol-5-yl)vinyl)thiazol-2-yl)-1-(pyridin-4- |
| | | | ylmethyl)-1H-pyrrole-2-carboxamide |
| C113 | 1 | 434 | 1-(2-(cyanomethyl)butyl)-N-(4-((R)-1-phenylpyrrolidin-2-yl)thiazol-2-yl)- |
| | | | 1H-pyrrole-2-carboxamide |
| Int. for | 2 | 730 | (R)-N-(4-(1-(4-(2-((tert-butyldiphenylsilyl)oxy)ethyl)phenyl)pyrrolidin-2- |
| A78 | | | yl)thiazol-2-yl)-1-((2-fluoropyridin-4-yl)methyl)-1H-pyrrole-2-carboxamide |
| B58 | 1 | 433 | (E)-N-(4-(2-(1-isopropyl-5-methyl-1H-imidazol-4-yl)vinyl)thiazol-2-yl)-1- |
| | | | (pyridin-4-ylmethyl)-1H-pyrrole-2-carboxamide |
| C115 | 1 | 472 | (R)-N-(4-(1-phenylpyrrolidin-2-yl)thiazol-2-yl)-1-(thieno[2,3-c]pyridin-3-yl)- |
| | | | 1H-pyrrole-2-carboxamide |
| Int. for | 1 | 633 | tert-butyl 3-(4-((R)-2-(2-(1-((2-fluoropyridin-4-yl)methyl)-1H-pyrrole-2- |
| A79 and | | | carboxamido)thiazol-4-yl)pyrrolidin-1-yl)phenoxy)pyrrolidine-1-carboxylate |
| A80 | | | |
| Common | 1 | 619 | tert-butyl (R)-3-(4-(2-(2-(1-((2-fluoropyridin-4-yl)methyl)-1H-pyrrole-2- |
| intermedi | | | carboxamido)thiazol-4-yl)pyrrolidin-1-yl)phenoxy)azetidine-1-carboxylate |
| ate | | | |
| Int. for | 1 | 645 | tert-butyl (R)-4-(4-(2-(2-(1-((2-fluoropyridin-4-yl)methyl)-1H-pyrrole-2- |
| A82 and | | | carboxamido)thiazol-4-yl)pyrrolidin-1-yl)phenoxy)-3,6-dihydropyridine- |
| A89 | | | 1(2H)-carboxylate |
| Int. for | 1 | 647 | tert-butyl (R)-3-(4-((R)-2-(2-(1-((2-fluoropyridin-4-yl)methyl)-1H-pyrrole-2- |
| A83 and | | | carboxamido)thiazol-4-yl)pyrrolidin-1-yl)phenoxy)piperidine-1-carboxylate |
| A87 | | | |
| Int. for | 1 | 647 | tert-butyl (S)-3-(4-((R)-2-(2-(1-((2-fluoropyridin-4-yl)methyl)-1H-pyrrole-2- |
| A84 and | | | carboxamido)thiazol-4-yl)pyrrolidin-1-yl)phenoxy)piperidine-1-carboxylate |
| A88 | | | |
| B59 | 1 | 420 | (E)-1-((2-fluoropyridin-4-yl)methyl)-N-(4-(2-(6-methylpyridin-2- |
| | | | yl)vinyl)thiazol-2-yl)-1H-pyrrole-2-carboxamide |
| Int. for | 1 | 581 | tert-butyl (R)-(2-fluoro-4-(2-(2-(1-((2-fluoropyridin-4-yl)methyl)-1H-pyrrole- |
| A93 | | | 2-carboxamido)thiazol-4-yl)pyrrolidin-1-yl)phenyl)carbamate |
| A94 | 2 | 453 | (R)-1-((2-fluoropyridin-4-yl)methyl)-N-(4-(1-(phenyl-d5)pyrrolidin-2- |
| | | | yl)thiazol-2-yl)-1H-pyrrole-2-carboxamide |
| A95 | 2 | 450 | (R)-1-((2-fluoropyridin-4-yl)methyl)-N-(4-(1-(phenyl-3,5-d2)pyrrolidin-2- |
| | | | yl)thiazol-2-yl)-1H-pyrrole-2-carboxamide |
| Int. for | 2 | 599 | tert-butyl (R)-(3,5-difluoro-4-(2-(2-(1-((2-fluoropyridin-4-yl)methyl)-1H- |
| A96 | | | pyrrole-2-carboxamido)thiazol-4-yl)pyrrolidin-1-yl)phenyl)carbamate |
| Int. for | 1 | 661 | tert-butyl (R)-4-(4-(2-(2-(1-((2-fluoropyridin-4-yl)methyl)-1H-pyrrole-2- |
| A97 | | | carboxamido)thiazol-4-yl)pyrrolidin-1-yl)phenoxy)-4-methylpiperidine-1- |
| | | | carboxylate |
| Int. for | 1 | 581 | tert-butyl (R)-(3-fluoro-4-(2-(2-(1-((2-fluoropyridin-4-yl)methyl)-1H-pyrrole- |
| A98 | | | 2-carboxamido)thiazol-4-yl)pyrrolidin-1-yl)phenyl)carbamate |
| Int. for | 1 | 599 | tert-butyl (R)-(2,6-difluoro-4-(2-(2-(1-((2-fluoropyridin-4-yl)methyl)-1H- |
| A99 | | | pyrrole-2-carboxamido)thiazol-4-yl)pyrrolidin-1-yl)phenyl)carbamate |
| Int. for | 1 | 665 | tert-butyl (R)-4-(3-fluoro-4-(2-(2-(1-((2-fluoropyridin-4-yl)methyl)-1H- |
| A100 | | | pyrrole-2-carboxamido)thiazol-4-yl)pyrrolidin-1-yl)phenoxy)piperidine-1- |
| | | | carboxylate |
| Int. for | 1 | 607 | tert-butyl (R)-(2-(4-(2-(2-(1-((2-fluoropyridin-4-yl)methyl)-1H-pyrrole-2- |
| A101, | | | carboxamido)thiazol-4-yl)pyrrolidin-1-yl)phenoxy)ethyl)carbamate |
| A104, | | | |
| A121 | | | |
| B60 | 1 | 460 | (E)-1-((2-fluoropyridin-4-yl)methyl)-N-(4-(2-(5,6,7,8-tetrahydroquinolin-2- |
| | | | yl)vinyl)thiazol-2-yl)-1H-pyrrole-2-carboxamide |
| Int. for | 1 | 665 | tert-butyl (R)-4-(2-fluoro-4-(2-(2-(1-((2-fluoropyridin-4-yl)methyl)-1H- |
| A102 | | | pyrrole-2-carboxamido)thiazol-4-yl)pyrrolidin-1-yl)phenoxy)piperidine-1- |
| | | | carboxylate |
| Int. for | 1 | 621 | tert-butyl (R)-(2-(4-(2-(2-(1-((2-fluoropyridin-4-yl)methyl)-1H-pyrrole-2- |
| A103 | | | carboxamido)thiazol-4-yl)pyrrolidin-1-yl)phenoxy)ethyl)(methyl)carbamate |
| and | | | |
| A117 | | | |
| Int. for | 1 | 436 | (E)-1-((2-fluoropyridin-4-yl)methyl)-N-(4-(2-(6-methoxypyridin-2- |
| B61 | | | yl)vinyl)thiazol-2-yl)-1H-pyrrole-2-carboxamide |
| B62 | 1 | 435 | (E)-N-(4-(2-(6,7-dihydro-5H-pyrrolo[1,2-a]imidazol-2-yl)vinyl)thiazol-2-yl)- |
| | | | 1-((2-fluoropyridin-4-yl)methyl)-1H-pyrrole-2-carboxamide |
| Int. for | 1 | 683 | tert-butyl (R)-4-(3,5-difluoro-4-(2-(2-(1-((2-fluoropyridin-4-yl)methyl)-1H- |
| A105 | | | pyrrole-2-carboxamido)thiazol-4-yl)pyrrolidin-1-yl)phenoxy)piperidine-1- |
| | | | carboxylate |
| Int. for | 1 | 683 | tert-butyl (R)-4-(2,6-difluoro-4-(2-(2-(1-((2-fluoropyridin-4-yl)methyl)-1H- |
| A106 | | | pyrrole-2-carboxamido)thiazol-4-yl)pyrrolidin-1-yl)phenoxy)piperidine-1- |
| | | | carboxylate |
| Int. for | 1 | 665 | tert-butyl 3-fluoro-4-(4-((R)-2-(2-(1-((2-fluoropyridin-4-yl)methyl)-1H- |
| A107 | | | pyrrole-2-carboxamido)thiazol-4-yl)pyrrolidin-1-yl)phenoxy)piperidine-1- |
| and | | | carboxylate |
| A108 | | | |
| B63 | 1 | 419 | (E)-N-(4-(2-(1-isopropyl-1H-imidazol-4-yl)vinyl)thiazol-2-yl)-1-(pyridin-3- |
| | | | ylmethyl)-1H-pyrrole-2-carboxamide |
| B64 | 1 | 448 | (E)-1-((2-fluoropyridin-4-yl)methyl)-N-(4-(2-(5-isopropylpyridin-2- |
| | | | yl)vinyl)thiazol-2-yl)-1H-pyrrole-2-carboxamide |
| B65 | 1 | 423 | (E)-N-(4-(2-(1-ethyl-1H-imidazol-4-yl)vinyl)thiazol-2-yl)-1-((2-fluoropyridin- |
| | | | 4-yl)methyl)-1H-pyrrole-2-carboxamide |
| B66 | 1 | 430 | (E)-N-(4-(2-(5-isopropylpyridin-2-yl)vinyl)thiazol-2-yl)-1-(pyridin-3- |
| | | | ylmethyl)-1H-pyrrole-2-carboxamide |
| B67 | 1 | 451 | (E)-1-((2-fluoropyridin-4-yl)methyl)-N-(4-(2-(2-isopropyl-1-methyl-1H- |
| | | | imidazol-4-yl)vinyl)thiazol-2-yl)-1H-pyrrole-2-carboxamide |
| B68 | 1 | 455 | (E)-1-((2,6-difluoropyridin-4-yl)methyl)-N-(4-(2-(1-isopropyl-1H-imidazol-4- |
| | | | yl)vinyl)thiazol-2-yl)-1H-pyrrole-2-carboxamide |
| B69 | 1 | 405 | (E)-N-(4-(2-(1-ethyl-1H-imidazol-4-yl)vinyl)thiazol-2-yl)-1-(pyridin-4- |
| | | | ylmethyl)-1H-pyrrole-2-carboxamide |
| Int. for | 2 | 606 | N-(4-((R)-1-(4-((R)-1-((tert-butyldimethylsilyl)oxy)ethyl)phenyl)pyrrolidin-2- |
| A111 | | | yl)thiazol-2-yl)-1-((2-fluoropyridin-4-yl)methyl)-1H-pyrrole-2-carboxamide |
| Int. for | 2 | 606 | N-(4-((R)-1-(4-((S)-1-((tert-butyldimethylsilyl)oxy)ethyl)phenyl)pyrrolidin-2- |
| A112 | | | yl)thiazol-2-yl)-1-((2-fluoropyridin-4-yl)methyl)-1H-pyrrole-2-carboxamide |
| Int. for | 2 | 592 | (R)-N-(4-(1-(4-(((tert-butyldimethylsilyl)oxy)methyl)phenyl)pyrrolidin-2- |
| A113 | | | yl)thiazol-2-yl)-1-((2-fluoropyridin-4-yl)methyl)-1H-pyrrole-2-carboxamide |
| Int. for | 2 | 632 | (R)-N-(4-(1-(4-(1-((tert-butyldimethylsilyl)oxy)cyclobutyl)phenyl)pyrrolidin- |
| A114 | | | 2-yl)thiazol-2-yl)-1-((2-fluoropyridin-4-yl)methyl)-1H-pyrrole-2- |
| | | | carboxamide |
| A115 | 2 | 444 | (R)-4-methyl-N-(4-(1-phenylpyrrolidin-2-yl)thiazol-2-yl)-1-(pyridin-4- |
| | | | ylmethyl)-1H-pyrrole-2-carboxamide |
| B70 | 1 | 459 | (E)-1-((2-fluoropyridin-4-yl)methyl)-N-(4-(2-(5-methylimidazo[1,2-a]pyridin- |
| | | | 2-yl)vinyl)thiazol-2-yl)-1H-pyrrole-2-carboxamide |
| B71 | 1 | 417 | (E)-N-(4-(2-(6,7-dihydro-5H-pyrrolo[1,2-a]imidazol-2-yl)vinyl)thiazol-2-yl)- |
| | | | 1-(pyridin-4-ylmethyl)-1H-pyrrole-2-carboxamide |
| B72 | 1 | 433 | (E)-N-(4-(2-(2-isopropyl-1-methyl-1H-imidazol-4-yl)vinyl)thiazol-2-yl)-1- |
| | | | (pyridin-4-ylmethyl)-1H-pyrrole-2-carboxamide |
| B73 | 1 | 487 | (E)-1-((2-fluoropyridin-4-yl)methyl)-N-(4-(2-(5-isopropylimidazo[1,2- |
| | | | a]pyridin-2-yl)vinyl)thiazol-2-yl)-1H-pyrrole-2-carboxamide |
| B74 | 1 | 437 | (E)-1-((2-fluoropyridin-4-yl)methyl)-N-(4-(2-(1-isopropyl-1H-pyrazol-3- |
| | | | yl)vinyl)thiazol-2-yl)-1H-pyrrole-2-carboxamide |
| B75 | 1 | 419 | (E)-N-(4-(2-(1-isopropyl-1H-pyrazol-3-yl)vinyl)thiazol-2-yl)-1-(pyridin-4- |
| | | | ylmethyl)-1H-pyrrole-2-carboxamide |
| B76 | 1 | 469 | (E)-N-(4-(2-(5-isopropylimidazo[1,2-a]pyridin-2-yl)vinyl)thiazol-2-yl)-1- |
| | | | (pyridin-4-ylmethyl)-1H-pyrrole-2-carboxamide |
| B77 | 1 | 441 | (E)-N-(4-(2-(5-methylimidazo[1,2-a]pyridin-2-yl)vinyl)thiazol-2-yl)-1- |
| | | | (pyridin-4-ylmethyl)-1H-pyrrole-2-carboxamide |
| C116 | 2 | 437 | (R)-1-(3-(oxetan-3-yl)propyl)-N-(4-(1-phenylpyrrolidin-2-yl)thiazol-2-yl)-1H- |
| | | | pyrrole-2-carboxamide |
| B78 | 1 | 473 | (E)-N-(4-(2-(5-ethylimidazo[1,2-a]pyridin-2-yl)vinyl)thiazol-2-yl)-1-((2- |
| | | | fluoropyridin-4-yl)methyl)-1H-pyrrole-2-carboxamide |
| B79 | 1 | 455 | (E)-N-(4-(2-(5-ethylimidazo[1,2-a]pyridin-2-yl)vinyl)thiazol-2-yl)-1-(pyridin- |
| | | | 4-ylmethyl)-1H-pyrrole-2-carboxamide |
| B80 | 1 | 433 | (E)-N-(4-(2-(1-isopropyl-4-methyl-1H-pyrazol-3-yl)vinyl)thiazol-2-yl)-1- |
| | | | (pyridin-4-ylmethyl)-1H-pyrrole-2-carboxamide |
| A120 | 1 | 506 | (R)-1-((2-fluoropyridin-4-yl)methyl)-N-(4-(1-(4-isopropoxyphenyl)pyrrolidin- |
| | | | 2-yl)thiazol-2-yl)-1H-pyrrole-2-carboxamide |
| B81 | 1 | 451 | (E)-1-((2-fluoropyridin-4-yl)methyl)-N-(4-(2-(5-isopropyl-1-methyl-1H- |
| | | | imidazol-4-yl)vinyl)thiazol-2-yl)-1H-pyrrole-2-carboxamide |
| B83 | 1 | 451 | (E)-1-((2-fluoropyridin-4-yl)methyl)-N-(4-(2-(1-isopropyl-4-methyl-1H- |
| | | | pyrazol-3-yl)vinyl)thiazol-2-yl)-1H-pyrrole-2-carboxamide |
| B84 | 1 | 451 | (E)-1-((2-fluoropyridin-4-yl)methyl)-N-(4-(2-(1-isopropyl-4-methyl-1H- |
| | | | pyrazol-5-yl)vinyl)thiazol-2-yl)-1H-pyrrole-2-carboxamide |
| B85 | 1 | 433 | (E)-N-(4-(2-(5-isopropyl-1-methyl-1H-imidazol-4-yl)vinyl)thiazol-2-yl)-1- |
| | | | (pyridin-4-ylmethyl)-1H-pyrrole-2-carboxamide |
| Int. for | 1 | 581 | tert-butyl (R)-(4-(2-(2-(1-((2,6-difluoropyridin-4-yl)methyl)-1H-pyrrole-2- |
| A122 | | | carboxamido)thiazol-4-yl)pyrrolidin-1-yl)phenyl)carbamate |
| Int. for | 1 | 672 | tert-butyl (R)-4-(2-cyano-4-(2-(2-(1-((2-fluoropyridin-4-yl)methyl)-1H- |
| A126 | | | pyrrole-2-carboxamido)thiazol-4-yl)pyrrolidin-1-yl)phenoxy)piperidine-1- |
| | | | carboxylate |
| A127 | 1 | 522 | (R)-1-((2-fluoropyridin-4-yl)methyl)-N-(4-(1-(4-(2- |
| | | | methoxyethoxy)phenyl)pyrrolidin-2-yl)thiazol-2-yl)-1H-pyrrole-2- |
| | | | carboxamide |
| Int. for | 1 | 677 | tert-butyl (R)-4-(4-(2-(2-(1-((2-fluoropyridin-4-yl)methyl)-1H-pyrrole-2- |
| A129 | | | carboxamido)thiazol-4-yl)pyrrolidin-1-yl)-2-methoxyphenoxy)piperidine-1- |
| | | | carboxylate |
| Int. for | 1 | 672 | tert-butyl (R)-4-(3-cyano-4-(2-(2-(1-((2-fluoropyridin-4-yl)methyl)-1H- |
| A133 | | | pyrrole-2-carboxamido)thiazol-4-yl)pyrrolidin-1-yl)phenoxy)piperidine-1- |
| | | | carboxylate |
| Int. for | 1 | 746 | (R)-N-(4-(1-(4-(2-((tert-butyldiphenylsilyl)oxy)ethoxy)phenyl)pyrrolidin-2- |
| A134 | | | yl)thiazol-2-yl)-1-((2-fluoropyridin-4-yl)methyl)-1H-pyrrole-2-carboxamide |
| A138 | 1 | 596 | (R)-N-(4-(1-(4-((1,1-dioxidotetrahydro-2H-thiopyran-4- |
| | | | yl)oxy)phenyl)pyrrolidin-2-yl)thiazol-2-yl)-1-((2-fluoropyridin-4-yl)methyl)- |
| | | | 1H-pyrrole-2-carboxamide |
| B86 | 2 | 449 | (E)-1-((2-fluoropyridin-4-yl)methyl)-N-(4-(2-(5,6,7,8-tetrahydroimidazo[1,5- |
| | | | a]pyridin-1-yl)vinyl)thiazol-2-yl)-1H-pyrrole-2-carboxamide |
| Int. for | 1 | 681 | tert-butyl (R)-4-(2-chloro-4-(2-(2-(1-((2-fluoropyridin-4-yl)methyl)-1H- |
| A140 | | | pyrrole-2-carboxamido)thiazol-4-yl)pyrrolidin-1-yl)phenoxy)piperidine-1- |
| | | | carboxylate |
| B87 | 1 | 466 | (E)-1-((2,6-difluoropyridin-4-yl)methyl)-N-(4-(2-(5-isopropylpyridin-2- |
| | | | yl)vinyl)thiazol-2-yl)-1H-pyrrole-2-carboxamide |
| D6 | 1 | 437 | (E)-N-(4-(2-(5-isopropylpyridin-2-yl)vinyl)thiazol-2-yl)-1-((tetrahydro-2H- |
| | | | pyran-4-yl)methyl)-1H-pyrrole-2-carboxamide |
| D7 | 1 | 426 | (E)-N-(4-(2-(1-isopropyl-1H-imidazol-4-yl)vinyl)thiazol-2-yl)-1-((tetrahydro- |
| | | | 2H-pyran-4-yl)methyl)-1H-pyrrole-2-carboxamide |
| A142 | 1 | 516 | (S)-1-((2-fluoropyridin-4-yl)methyl)-N-(4-(1-(4- |
| | | | (trifluoromethyl)phenyl)pyrrolidin-2-yl)thiazol-2-yl)-1H-pyrrole-2- |
| | | | carboxamide |
| B88 | 1 | 410 | (E)-1-((2-fluoropyridin-4-yl)methyl)-N-(4-(2-(5-methylisoxazol-3- |
| | | | yl)vinyl)thiazol-2-yl)-1H-pyrrole-2-carboxamide |
| B89 | 1 | 424 | (E)-N-(4-(2-(5-ethylisoxazol-3-yl)vinyl)thiazol-2-yl)-1-((2-fluoropyridin-4- |
| | | | yl)methyl)-1H-pyrrole-2-carboxamide |
| B90 | 1 | 438 | (E)-1-((2-fluoropyridin-4-yl)methyl)-N-(4-(2-(5-isopropylisoxazol-3- |
| | | | yl)vinyl)thiazol-2-yl)-1H-pyrrole-2-carboxamide |
| Int. for | 1 | 535 | tert-butyl (4-((R)-2-(2-(1-((S)-3-cyano-2-methylpropyl)-1H-pyrrole-2- |
| C117 | | | carboxamido)thiazol-4-yl)pyrrolidin-1-yl)phenyl)carbamate |
| Int. for | 1 | 681 | tert-butyl (R)-4-(3-chloro-4-(2-(2-(1-((2-fluoropyridin-4-yl)methyl)-1H- |
| A143 | | | pyrrole-2-carboxamido)thiazol-4-yl)pyrrolidin-1-yl)phenoxy)piperidine-1- |
| | | | carboxylate |
| B91 | 1 | 520 | (E)-N-(4-(2-(1-(1-acetylpiperidin-4-yl)-1H-imidazol-4-yl)vinyl)thiazol-2-yl)- |
| | | | 1-((2-fluoropyridin-4-yl)methyl)-1H-pyrrole-2-carboxamide |
| B92 | 1 | 479 | (E)-1-((2-fluoropyridin-4-yl)methyl)-N-(4-(2-(1-(tetrahydro-2H-pyran-4-yl)- |
| | | | 1H-imidazol-4-yl)vinyl)thiazol-2-yl)-1H-pyrrole-2-carboxamide |
| B93 | 1 | 462 | (E)-N-(4-(2-(2-cyano-1-isopropyl-1H-imidazol-4-yl)vinyl)thiazol-2-yl)-1-((2- |
| | | | fluoropyridin-4-yl)methyl)-1H-pyrrole-2-carboxamide |
| A149 | 1 | 473 | (R)-4-cyano-1-((2-fluoropyridin-4-yl)methyl)-N-(4-(1-phenylpyrrolidin-2- |
| and Int. | | | yl)thiazol-2-yl)-1H-pyrrole-2-carboxamide |
| for A152 | | | |
| B94 | 1 | 451 | (E)-1-((2-fluoropyridin-4-yl)methyl)-N-(4-(2-(3-isopropyl-1-methyl-1H- |
| | | | pyrazol-5-yl)vinyl)thiazol-2-yl)-1H-pyrrole-2-carboxamide |
| B95 | 2 | 437 | (E)-1-((2-fluoropyridin-4-yl)methyl)-N-(4-(2-(1-isopropyl-1H-imidazol-2- |
| | | | yl)vinyl)thiazol-2-yl)-1H-pyrrole-2-carboxamide |
| B96 | 1 | 451 | (E)-1-((2-fluoropyridin-4-yl)methyl)-N-(4-(2-(5-isopropyl-1-methyl-1H- |
| | | | pyrazol-3-yl)vinyl)thiazol-2-yl)-1H-pyrrole-2-carboxamide |
| Int. for | 1 | 648 | tert-butyl (R)-4-((6-(2-(2-(1-((2-fluoropyridin-4-yl)methyl)-1H-pyrrole-2- |
| A154 | | | carboxamido)thiazol-4-yl)pyrrolidin-1-yl)pyridin-3-yl)oxy)piperidine-1- |
| and | | | carboxylate |
| A156 | | | |
| Int. for | 1 | 648 | tert-butyl (R)-4-((5-(2-(2-(1-((2-fluoropyridin-4-yl)methyl)-1H-pyrrole-2- |
| A155 | | | carboxamido)thiazol-4-yl)pyrrolidin-1-yl)pyridin-2-yl)oxy)piperidine-1- |
| and | | | carboxylate |
| A159 | | | |
| A157 | 1 | 536 | (R)-1-((2-fluoropyridin-4-yl)methyl)-N-(4-(1-(4-(thietan-3- |
| and Int. | | | yloxy)phenyl)pyrrolidin-2-yl)thiazol-2-yl)-1H-pyrrole-2-carboxamide |
| for A158 | | | |
| B97 | 1 | 463 | (E)-1-((2-fluoropyridin-4-yl)methyl)-N-(4-(2-(7-methyl-5,6,7,8- |
| | | | tetrahydroimidazo[1,5-a]pyridin-1-yl)vinyl)thiazol-2-yl)-1H-pyrrole-2- |
| | | | carboxamide |
| B98 | 1 | 463 | (E)-1-((2-fluoropyridin-4-yl)methyl)-N-(4-(2-(6-methyl-5,6,7,8- |
| | | | tetrahydroimidazo[1,5-a]pyridin-1-yl)vinyl)thiazol-2-yl)-1H-pyrrole-2- |
| | | | carboxamide |
| Int. for | 1 | 645 | tert-butyl (R)-4-(4-(2-(2-(1-((2-fluoropyridin-4-yl)methyl)-1H-pyrrole-2- |
| A160 | | | carboxamido)thiazol-4-yl)pyrrolidin-1-yl)benzyl)piperidine-1-carboxylate |
| Int. for | 1 | 659 | tert-butyl 4-(1-(4-((R)-2-(2-(1-((2-fluoropyridin-4-yl)methyl)-1H-pyrrole-2- |
| A161 | | | carboxamido)thiazol-4-yl)pyrrolidin-1-yl)phenyl)ethyl)piperidine-1- |
| | | | carboxylate |
| B99 | 1 | 451 | (E)-1-((2-fluoropyridin-4-yl)methyl)-N-(4-(2-(1-isopropyl-1H-imidazol-4- |
| | | | yl)vinyl)thiazol-2-yl)-4-methyl-1H-pyrrole-2-carboxamide |
| A162 | 1 | 456 | 1-((2-fluoropyridin-4-yl)methyl)-N-(4-(3-(pyridin-2-yl)phenyl)thiazol-2-yl)- |
| | | | 1H-pyrrole-2-carboxamide |
| C118 | 1 | 447 | (R)-1-(3-fluorobenzyl)-N-(4-(1-phenylpyrrolidin-2-yl)thiazol-2-yl)-1H- |
| | | | pyrrole-2-carboxamide |
| C119 | 1 | 447 | (R)-1-(4-fluorobenzyl)-N-(4-(1-phenylpyrrolidin-2-yl)thiazol-2-yl)-1H- |
| | | | pyrrole-2-carboxamide |
| B100 | 2 | 449 | (E)-1-((2-fluoropyridin-4-yl)methyl)-N-(4-(2-(5,6,7,8-tetrahydroimidazo[1,5- |
| | | | a]pyridin-3-yl)vinyl)thiazol-2-yl)-1H-pyrrole-2-carboxamide |
| B101 | 1 | 463 | (E)-1-((2-fluoropyridin-4-yl)methyl)-N-(4-(2-(5-methyl-5,6,7,8- |
| | | | tetrahydroimidazo[1,5-a]pyridin-1-yl)vinyl)thiazol-2-yl)-1H-pyrrole-2- |
| | | | carboxamide |
| B102 | 1 | 463 | (E)-1-((2-fluoropyridin-4-yl)methyl)-N-(4-(2-(8-methyl-5,6,7,8- |
| | | | tetrahydroimidazo[1,5-a]pyridin-1-yl)vinyl)thiazol-2-yl)-1H-pyrrole-2- |
| | | | carboxamide |
| Int. for | 1 | 577 | tert-butyl (R)-(4-(2-(2-(1-((2-fluoropyridin-4-yl)methyl)-4-methyl-1H- |
| A163, | | | pyrrole-2-carboxamido)thiazol-4-yl)pyrrolidin-1-yl)phenyl)carbamate |
| A164, | | | |
| and | | | |
| A172 | | | |
| Int. for | 1 | 509 | ethyl (E)-4-(2-(2-(1-((2-fluoropyridin-4-yl)methyl)-1H-pyrrole-2- |
| B103 | | | carboxamido)thiazol-4-yl)vinyl)-1-isopropyl-1H-imidazole-2-carboxylate |
| Int. for | 1 | 661 | tert-butyl (R)-4-(4-(2-(2-(1-((2-fluoropyridin-4-yl)methyl)-4-methyl-1H- |
| A164 | | | pyrrole-2-carboxamido)thiazol-4-yl)pyrrolidin-1-yl)phenoxy)piperidine-1- |
| and | | | carboxylate |
| A165 | | | |
| B104 | 1 | 471 | (E)-1-((2-fluoropyridin-4-yl)methyl)-N-(4-(2-(1-phenyl-1H-imidazol-4- |
| | | | yl)vinyl)thiazol-2-yl)-1H-pyrrole-2-carboxamide |
| B105 | 1 | 485 | (E)-N-(4-(2-(1-benzyl-1H-imidazol-4-yl)vinyl)thiazol-2-yl)-1-((2- |
| | | | fluoropyridin-4-yl)methyl)-1H-pyrrole-2-carboxamide |
| A167 | 1 | 487 | 1-((2-fluoropyridin-4-yl)methyl)-N-(4-(3-(1-isopropyl-1H-imidazol-4- |
| | | | yl)phenyl)thiazol-2-yl)-1H-pyrrole-2-carboxamide |
| A168 | 1 | 456 | 1-((2-fluoropyridin-4-yl)methyl)-N-(4-(2-(pyridin-2-yl)phenyl)thiazol-2-yl)- |
| | | | 1H-pyrrole-2-carboxamide |
| B106 | 1 | 451 | (E)-1-((2-fluoropyridin-4-yl)methyl)-N-(4-(3-(1-isopropyl-1H-imidazol-4- |
| | | | yl)allyl)thiazol-2-yl)-1H-pyrrole-2-carboxamide |
| | | | Skipped amine synthesis |
| B107 | 1 | 448 | (E)-N-(4-(2-(1-(1-cyanoethyl)-1H-imidazol-4-yl)vinyl)thiazol-2-yl)-1-((2- |
| and Int. | | | fluoropyridin-4-yl)methyl)-1H-pyrrole-2-carboxamide |
| for B113 | | | |
| B108 | 1 | 448 | (E)-N-(4-(2-(1-(2-cyanoethyl)-1H-imidazol-4-yl)vinyl)thiazol-2-yl)-1-((2- |
| and Int. | | | fluoropyridin-4-yl)methyl)-1H-pyrrole-2-carboxamide |
| for B109 | | | |
| B110 | 1 | 462 | (E)-N-(4-(2-(1-(1-cyanopropan-2-yl)-1H-imidazol-4-yl)vinyl)thiazol-2-yl)-1- |
| and Int. | | | ((2-fluoropyridin-4-yl)methyl)-1H-pyrrole-2-carboxamide |
| for B111 | | | |
| A169 | 1 | 487 | 1-((2-fluoropyridin-4-yl)methyl)-N-(4-(2-(1-isopropyl-1H-imidazol-4- |
| | | | yl)phenyl)thiazol-2-yl)-1H-pyrrole-2-carboxamide |
| A170 | 1 | 564 | (R)-1-((2-fluoropyridin-4-yl)methyl)-N-(4-(1-(4-((tetrahydro-2H-thiopyran-4- |
| | | | yl)oxy)phenyl)pyrrolidin-2-yl)thiazol-2-yl)-1H-pyrrole-2-carboxamide |
| B112 | 1 | 434 | (E)-N-(4-(2-(1-(cyanomethyl)-1H-imidazol-4-yl)vinyl)thiazol-2-yl)-1-((2- |
| and Int. | | | fluoropyridin-4-yl)methyl)-1H-pyrrole-2-carboxamide |
| for B115 | | | |
| Int. for | 1 | 525 | (E)-1-((2-fluoropyridin-4-yl)methyl)-N-(4-(2-(1-((2- |
| B114 | | | (trimethylsilyl)ethoxy)methyl)-1H-imidazol-4-yl)vinyl)thiazol-2-yl)-1H- |
| | | | pyrrole-2-carboxamide |
| C120 | 1 | 443 | (R)-1-(3-methylbenzyl)-N-(4-(1-phenylpyrrolidin-2-yl)thiazol-2-yl)-1H- |
| | | | pyrrole-2-carboxamide |
| C121 | 1 | 507 | (R)-1-(3-bromobenzyl)-N-(4-(1-phenylpyrrolidin-2-yl)thiazol-2-yl)-1H- |
| | | | pyrrole-2-carboxamide |
| Int. for | 1 | 627 | tert-butyl (R)-3-((4-(2-(2-(1-((2-fluoropyridin-4-yl)methyl)-1H-pyrrole-2- |
| A176 | | | carboxamido)thiazol-4-yl)pyrrolidin-1-yl)phenyl)ethynyl)azetidine-1- |
| | | | carboxylate |
| Int. for | 1 | 564 | tert-butyl (R)-(6-(2-(2-(1-((2-fluoropyridin-4-yl)methyl)-1H-pyrrole-2- |
| A175 | | | carboxamido)thiazol-4-yl)pyrrolidin-1-yl)pyridin-3-yl)carbamate |
| C122 | 1 | 463 | (R)-1-(3-chlorobenzyl)-N-(4-(1-phenylpyrrolidin-2-yl)thiazol-2-yl)-1H- |
| | | | pyrrole-2-carboxamide |
| B116 | 1 | 451 | (E)-1-((2-fluoropyridin-4-yl)methyl)-N-(4-(2-(1-isopropyl-5-methyl-1H- |
| | | | imidazol-4-yl)vinyl)thiazol-2-yl)-1H-pyrrole-2-carboxamide |
| B117 | 1 | 437 | (E)-N-(4-(2-(1-ethyl-5-methyl-1H-imidazol-4-yl)vinyl)thiazol-2-yl)-1-((2- |
| | | | fluoropyridin-4-yl)methyl)-1H-pyrrole-2-carboxamide |
| B119 | 1 | 509 | ethyl (E)-2-(4-(2-(2-(1-((2-fluoropyridin-4-yl)methyl)-1H-pyrrole-2- |
| and Int. | | | carboxamido)thiazol-4-yl)vinyl)-5-methyl-1H-imidazol-1-yl)propanoate |
| for B118 | | | |
| Int. for | 1 | 578 | tert-butyl (R)-(6-(2-(2-(1-((2-fluoropyridin-4-yl)methyl)-4-methyl-1H- |
| A180 | | | pyrrole-2-carboxamido)thiazol-4-yl)pyrrolidin-1-yl)pyridin-3-yl)carbamate |
| C123 | 1 | 457 | 1-((3,3-difluorocyclopentyl)methyl)-N-(4-((R)-1-phenylpyrrolidin-2- |
| | | | yl)thiazol-2-yl)-1H-pyrrole-2-carboxamide |
| B120 | 1 | 481 | methyl (E)-2-(4-(2-(2-(1-((2-fluoropyridin-4-yl)methyl)-1H-pyrrole-2- |
| and Int. | | | carboxamido)thiazol-4-yl)vinyl)-5-methyl-1H-imidazol-1-yl)acetate |
| for B121 | | | |
| B122 | 1 | 477 | (E)-1-((2-fluoropyridin-4-yl)methyl)-N-(4-(2-(1-(2,2,2-trifluoroethyl)-1H- |
| | | | imidazol-4-yl)vinyl)thiazol-2-yl)-1H-pyrrole-2-carboxamide |
| B123 | 1 | 451 | (E)-1-((2-fluoropyridin-4-yl)methyl)-N-(4-(2-(1-(oxetan-3-yl)-1H-imidazol-4- |
| | | | yl)vinyl)thiazol-2-yl)-1H-pyrrole-2-carboxamide |
| B124 | 1 | 465 | (E)-1-((2-fluoropyridin-4-yl)methyl)-N-(4-(2-(1-(tetrahydrofuran-3-yl)-1H- |
| | | | imidazol-4-yl)vinyl)thiazol-2-yl)-1H-pyrrole-2-carboxamide |
| D8 | 1 | 436 | (E)-1-(3-fluorobenzyl)-N-(4-(2-(1-isopropyl-1H-imidazol-4-yl)vinyl)thiazol- |
| | | | 2-yl)-1H-pyrrole-2-carboxamide |
| A185 | 1 | 526 | (R)-4-bromo-1-((2-fluoropyridin-4-yl)methyl)-N-(4-(1-phenylpyrrolidin-2- |
| | | | yl)thiazol-2-yl)-1H-pyrrole-2-carboxamide |
| C124 | 1 | 421 | (R)-1-(cyclopentylmethyl)-N-(4-(1-phenylpyrrolidin-2-yl)thiazol-2-yl)-1H- |
| | | | pyrrole-2-carboxamide |
| A189 | | 466 | (R)-4-fluoro-1-((2-fluoropyridin-4-yl)methyl)-N-(4-(1-phenylpyrrolidin-2- |
| | | | yl)thiazol-2-yl)-1H-pyrrole-2-carboxamide |
| B125 | | 438 | (E)-1-(2-fluoropyridin-4-yl)methyl)-N-(4-(2-(5-isopropyloxazol-4- |
| | | | yl)vinyl)thiazol-2-yl)-1H-pyrrole-2-carboxamide |
| B126 | | 424 | (E)-N-(4-(2-(5-ethyloxazol-4-yl)vinyl)thiazol-2-yl)-1-((2-fluoropyridin-4- |
| | | | yl)methyl)-1H-pyrrole-2-carboxamide |
| B127 | | 449 | (E)-N-(4-(2-(1-cyclobutyl-1H-imidazol-4-yl)vinyl)thiazol-2-yl)-1-((2- |
| | | | fluoropyridin-4-yl)methyl)-1H-pyrrole-2-carboxamide |
| B128 | | 435 | (E)-N-(4-(2-(1-cyclopropyl-1H-imidazol-4-yl)vinyl)thiazol-2-yl)-1-((2- |
| | | | fluoropyridin-4-yl)methyl)-1H-pyrrole-2-carboxamide |
| B129 | | 471 | (E)-N-(4-(2-(5-chloro-1-isopropyl-1H-imidazol-4-yl)vinyl)thiazol-2-yl)-1-((2- |
| | | | fluoropyridin-4-yl)methyl)-1H-pyrrole-2-carboxamide |
| A191 | | 482 | (R)-4-chloro-1-((2-fluoropyridin-4-yl)methyl)-N-(4-(1-phenylpyrrolidin-2- |
| | | | yl)thiazol-2-yl)-1H-pyrrole-2-carboxamide |
| B130 | | 440 | (E)-1-((2-fluoropyridin-4-yl)methyl)-N-(4-(2-(5-(methoxymethyl)oxazol-4- |
| | | | yl)vinyl)thiazol-2-yl)-1H-pyrrole-2-carboxamide |
| B131 | | 452 | (E)-N-(4-(2-(5-(tert-butyl)oxazol-4-yl)vinyl)thiazol-2-yl)-1-((2-fluoropyridin- |
| | | | 4-yl)methyl)-1H-pyrrole-2-carboxamide |
| B132 | | 436 | (E)-N-(4-(2-(5-cyclopropyloxazol-4-yl)vinyl)thiazol-2-yl)-1-((2-fluoropyridin- |
| | | | 4-yl)methyl)-1H-pyrrole-2-carboxamide |
| B133 | | 450 | (E)-N-(4-(2-(5-cyclobutyloxazol-4-yl)vinyl)thiazol-2-yl)-1-((2-fluoropyridin- |
| | | | 4-yl)methyl)-1H-pyrrole-2-carboxamide |
| B134 | | 451 | (E)-N-(4-(2-(1-(tert-butyl)-1H-imidazol-4-yl)vinyl)thiazol-2-yl)-1-((2- |
| | | | fluoropyridin-4-yl)methyl)-1H-pyrrole-2-carboxamide |
| C125 | | 419 | (R)-1-(furan-3-ylmethyl)-N-(4-(1-phenylpyrrolidin-2-yl)thiazol-2-yl)-1H- |
| | | | pyrrole-2-carboxamide |
| C126 | | 419 | (R)-1-(furan-2-ylmethyl)-N-(4-(1-phenylpyrrolidin-2-yl)thiazol-2-yl)-1H- |
| | | | pyrrole-2-carboxamide |

Modifications after Amide Coupling

### Route 1:

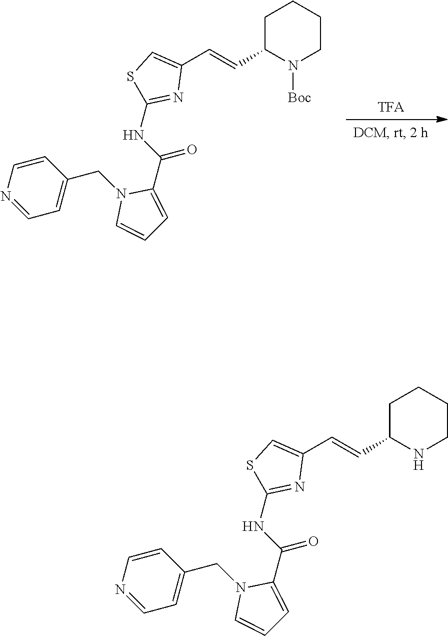 [SMILES: CC(C)(C)OC(=O)N1CCCC[C@H]1/C=C/c1csc(NC(=O)c2cccn2Cc2ccncc2)n1 ; "DCM, rt, 2 h" ; "TFA" ; O=C(Nc1nc(/C=C/[C@@H]2CCCCN2)cs1)c1cccn1Cc1ccncc1]

A 50 mL vial with stir bar was charged with tert-butyl (2S)-2-(2-[2-[1-(pyridin-4-ylmethyl)pyrrole-2-amido]-1,3-thiazol-4-yl]ethenyl)piperidine-1-carboxylate (60.00 mg, 0.12 mmol, 1.00 equiv) and DCM (1.00 mL), TEA (1 ml) was added. The vial was capped and placed in a room temperature bath. The reaction mixture was stirred at room temperature for 1 h. The resulting solution was concentrated in vacuo. The pH value of the solution was adjusted to 8 with NaHCO₃ (aq). The resulting solution was extracted with EtOAc (3×30 mL). The combined organic layers were dried over Na₂SO₄, filtered and concentrated in vacuo. The resulting crude material was purified via RP column to yield the desired product.

The following compounds were prepared via a similar method:

| | Observed | |
|---|---|---|
| | molecular | |
| | ion | Compound name |
| E18 | 394 | (S,E)-N-(4-(2-(piperidin-2-yl)vinyl)thiazol-2-yl)-1-(pyridin-4-ylmethyl)- |
| | | 1H-pyrrole-2-carboxamide |
| B7 | 442 | (E)-1-(pyridin-4-ylmethyl)-N-(4-(2-(1,2,3,4-tetrahydroquinolin-2- |
| | | yl)vinyl)thiazol-2-yl)-1H-pyrrole-2-carboxamide |
| E16 | 394 | (R,E)-N-(4-(2-(piperidin-2-yl)vinyl)thiazol-2-yl)-1-(pyridin-4-ylmethyl)- |
| | | 1H-pyrrole-2-carboxamide |
| A6 | 529 | (R)-N-(4-(1-(4-(piperidin-4-yloxy)phenyl)pyrrolidin-2-yl)thiazol-2-yl)-1- |
| | | (pyridin-4-ylmethyl)-1H-pyrrole-2-carboxamide |
| A44 | 459 | (R)-N-(4-(1-(4-(aminomethyl)phenyl)pyrrolidin-2-yl)thiazol-2-yl)-1- |
| | | (pyridin-4-ylmethyl)-1H-pyrrole-2-carboxamide |
| A49 | 473 | (R)-N-(4-(1-(4-((methylamino)methyl)phenyl)pyrrolidin-2-yl)thiazol-2- |
| | | yl)-1-(pyridin-4-ylmethyl)-1H-pyrrole-2-carboxamide |
| A51 | 513 | (R)-N-(4-(1-(4-(piperidin-4-yl)phenyl)pyrrolidin-2-yl)thiazol-2-yl)-1- |
| | | (pyridin-4-ylmethyl)-1H-pyrrole-2-carboxamide |
| B42 | 377 | (E)-N-(4-(2-(1H-imidazol-4-yl)vinyl)thiazol-2-yl)-1-(pyridin-4-ylmethyl)- |
| | | 1H-pyrrole-2-carboxamide |
| A59 | 513 | N-(4-((2R)-1-(4-(piperidin-2-yl)phenyl)pyrrolidin-2-yl)thiazol-2-yl)-1- |
| | | (pyridin-4-ylmethyl)-1H-pyrrole-2-carboxamide |
| C100 | 422 | 1-((S)-3-cyano-2-hydroxypropyl)-N-(4-((R)-1-phenylpyrrolidin-2- |
| | | yl)thiazol-2-yl)-1H-pyrrole-2-carboxamide |
| A61 | 513 | N-(4-((2R)-1-(4-(piperidin-3-yl)phenyl)pyrrolidin-2-yl)thiazol-2-yl)-1- |
| | | (pyridin-4-ylmethyl)-1H-pyrrole-2-carboxamide |
| A67 | 473 | N-(4-((R)-1-(4-((R)-1-aminoethyl)phenyl)pyrrolidin-2-yl)thiazol-2-yl)-1- |
| | | (pyridin-4-ylmethyl)-1H-pyrrole-2-carboxamide |
| A178 | 562 | azetidin-3-yl (R)-(4-(2-(2-(1-((2-fluoropyridin-4-yl)methyl)-1H-pyrrole-2- |
| | | carboxamido)thiazol-4-yl)pyrrolidin-1-yl)phenyl)carbamate |
| A74 | 491 | N-(4-((R)-1-(4-((S)-1-aminoethyl)phenyl)pyrrolidin-2-yl)thiazol-2-yl)-1- |
| | | ((2-fluoropyridin-4-yl)methyl)-1H-pyrrole-2-carboxamide |
| A72 | 491 | N-(4-((R)-1-(4-((R)-1-aminoethyl)phenyl)pyrrolidin-2-yl)thiazol-2-yl)-1- |
| | | ((2-fluoropyridin-4-yl)methyl)-1H-pyrrole-2-carboxamide |
| A71 | 473 | N-(4-((R)-1-(4-((S)-1-aminoethyl)phenyl)pyrrolidin-2-yl)thiazol-2-yl)-1- |
| | | (pyridin-4-ylmethyl)-1H-pyrrole-2-carboxamide |
| C112 | 422 | 1-((R)-3-cyano-2-hydroxypropyl)-N-(4-((R)-1-phenylpyrrolidin-2- |
| | | yl)thiazol-2-yl)-1H-pyrrole-2-carboxamide |
| A80 | 533 | 1-((2-fluoropyridin-4-yl)methyl)-N-(4-((2R)-1-(4-(pyrrolidin-3- |
| | | yloxy)phenyl)pyrrolidin-2-yl)thiazol-2-yl)-1H-pyrrole-2-carboxamide |
| A85 | 519 | (R)-N-(4-(1-(4-(azetidin-3-yloxy)phenyl)pyrrolidin-2-yl)thiazol-2-yl)-1- |
| | | ((2-fluoropyridin-4-yl)methyl)-1H-pyrrole-2-carboxamide |
| A150 | 598 | (R)-1-((2-fluoropyridin-4-yl)methyl)-N-(4-(1-(4-((1-sulfamoylazetidin-3- |
| | | yl)oxy)phenyl)pyrrolidin-2-yl)thiazol-2-yl)-1H-pyrrole-2-carboxamide |
| A89 | 545 | (R)-1-((2-fluoropyridin-4-yl)methyl)-N-(4-(1-(4-((1,2,3,6- |
| | | tetrahydropyridin-4-yl)oxy)phenyl)pyrrolidin-2-yl)thiazol-2-yl)-1H- |
| | | pyrrole-2-carboxamide |
| A87 | 547 | 1-((2-fluoropyridin-4-yl)methyl)-N-(4-((R)-1-(4-(((R)-piperidin-3- |
| | | yl)oxy)phenyl)pyrrolidin-2-yl)thiazol-2-yl)-1H-pyrrole-2-carboxamide |
| A88 | 547 | 1-((2-fluoropyridin-4-yl)methyl)-N-(4-((R)-1-(4-(((S)-piperidin-3- |
| | | yl)oxy)phenyl)pyrrolidin-2-yl)thiazol-2-yl)-1H-pyrrole-2-carboxamide |
| A101 | 507 | (R)-N-(4-(1-(4-(2-aminoethoxy)phenyl)pyrrolidin-2-yl)thiazol-2-yl)-1-((2- |
| | | fluoropyridin-4-yl)methyl)-1H-pyrrole-2-carboxamide |
| A103 | 521 | (R)-1-((2-fluoropyridin-4-yl)methyl)-N-(4-(1-(4-(2- |
| | | (methylamino)ethoxy)phenyl)pyrrolidin-2-yl)thiazol-2-yl)-1H-pyrrole-2- |
| | | carboxamide |
| A107 | 565 | N-(4-((2R)-1-(4-((3-fluoropiperidin-4-yl)oxy)phenyl)pyrrolidin-2- |
| | | yl)thiazol-2-yl)-1-((2-fluoropyridin-4-yl)methyl)-1H-pyrrole-2- |
| | | carboxamide |
| B114 | 395 | (E)-N-(4-(2-(1H-imidazol-4-yl)vinyl)thiazol-2-yl)-1-((2-fluoropyridin-4- |
| | | yl)methyl)-1H-pyrrole-2-carboxamide |

### Route 2:

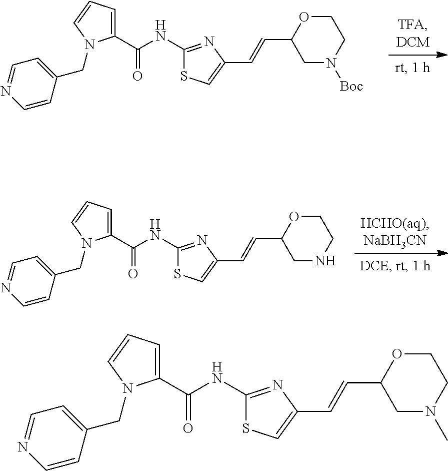

The Boc group was removed as described in route 1.

A 50 mL vial with stir bar was charged with N-[4-[(E)-2-(morpholin-2-yl)ethenyl]-1,3-thiazol-2-yl]-1-(pyridin-4-ylmethyl)pyrrole-2-carboxamide (100.00 mg, 0.25 mmol, 1.00 equiv), HCHO (37%, 101.35 mg, 1.25 mmol, 5.00 equiv) and DCE (5.00 mL), NaBH₃CN (31.42 mg, 0.50 mmol, 2.00 equiv) was added. The vial was capped and placed in a room temperature bath. The reaction mixture was stirred at room temperature for 2 h. The reaction was then quenched by water (10 mL). The resulting solution was extracted with ethyl acetate (3×10 mL). The combined organic layers were dried over Na₂SO₄, filtered and concentrated in vacuo. The resulting crude material was purified via RP column to yield the desired product.

The following compounds were prepared via a similar method:

| | Observed | |
|---|---|---|
| | molecular ion | Compound name |
| B6 | 410 | (E)-N-(4-(2-(4-methylmorpholin-2-yl)vinyl)thiazol-2-yl)-1-(pyridin-4-ylmethyl)-1H- |
| | | pyrrole-2-carboxamide |
| B8 | 456 | (E)-N-(4-(2-(1-methyl-1,2,3,4-tetrahydroquinolin-2-yl)vinyl)thiazol-2-yl)-1-(pyridin- |
| | | 4-ylmethyl)-1H-pyrrole-2-carboxamide |
| A8 | 543 | (R)-N-(4-(1-(4-((1-methylpiperidin-4-yl)oxy)phenyl)pyrrolidin-2-yl)thiazol-2-yl)-1- |
| | | (pyridin-4-ylmethyl)-1H-pyrrole-2-carboxamide |
| A173 | 576 | 1-methylazetidin-3-yl (R)-(4-(2-(2-(1-((2-fluoropyridin-4-yl)methyl)-1H-pyrrole-2- |
| | | carboxamido)thiazol-4-yl)pyrrolidin-1-yl)phenyl)carbamate |

### Route 3:

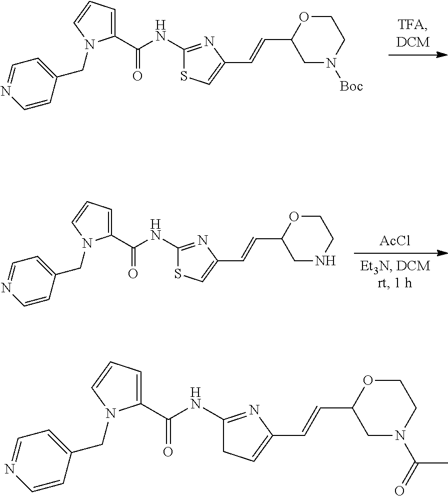

The Boc group was removed as described in route 1.

A 50 mL vial with stir bar was charged with N-[4-[(E)-2-(morpholin-2-yl)ethenyl]-1,3-thiazol-2-yl]-1-(pyridin-4-ylmethyl)pyrrole-2-carboxamide (100.00 mg, 0.25 mmol, 1.00 equiv), Et₃N (75.76 mg, 0.75 mmol, 3.00 equiv) and DCM (5.00 mL), acetyl chloride (23.82 mg, 0.30 mmol, 1.20 equiv) was added at 0° C. The vial was capped and placed in a room temperature bath. The reaction mixture was stirred at room temperature for 1 h. The reaction mixture was poured into DCM (15 ml) and washed with brine (1×20 mL). The organic layer was then dried over Na₂SO₄, filtered and concentrated in vacuo. The resulting crude material was purified via RP column to yield the desired product.

The following compounds were prepared via a similar method:

| | Observed | |
|---|---|---|
| | molecular | |
| | ion | Compound name |
| B10 | 438 | (E)-N-(4-(2-(4-acetylmorpholin-2-yl)vinyl)thiazol-2-yl)-1-(pyridin-4-ylmethyl)-1H- |
| | | pyrrole-2-carboxamide |
| E15 | 396 | (E)-N-(4-(3-(N-methylacetamido)prop-1-en-1-yl)thiazol-2-yl)-1-(pyridin-4-ylmethyl)- |
| | | 1H-pyrrole-2-carboxamide |
| E30 | 382 | (E)-N-(4-(3-acetamidoprop-1-en-1-yl)thiazol-2-yl)-1-(pyridin-4-ylmethyl)-1H-pyrrole-2- |
| | | carboxamide |
| E11 | 436 | (E)-N-(4-(2-(1-acetylpiperidin-2-yl)vinyl)thiazol-2-yl)-1-(pyridin-4-ylmethyl)-1H-pyrrole- |
| | | 2-carboxamide |
| E54 | 444 | (E)-N-(4-(3-benzamidoprop-1-en-1-yl)thiazol-2-yl)-1-(pyridin-4-ylmethyl)-1H-pyrrole-2- |
| | | carboxamide |
| A7 | 570 | (R)-N-(4-(1-(4-((1-acetylpiperidin-4-yl)oxy)phenyl)pyrrolidin-2-yl)thiazol-2-yl)-1- |
| | | (pyridin-4-ylmethyl)-1H-pyrrole-2-carboxamide |
| A45 | 501 | (R)-N-(4-(1-(4-(acetamidomethyl)phenyl)pyrrolidin-2-yl)thiazol-2-yl)-1-(pyridin-4- |
| | | ylmethyl)-1H-pyrrole-2-carboxamide |
| A46 | 487 | (R)-N-(4-(1-(4-acetamidophenyl)pyrrolidin-2-yl)thiazol-2-yl)-1-(pyridin-4-ylmethyl)-1H- |
| | | pyrrole-2-carboxamide |
| A55 | 555 | (R)-N-(4-(1-(4-(1-acetylpiperidin-4-yl)phenyl)pyrrolidin-2-yl)thiazol-2-yl)-1-(pyridin-4- |
| | | ylmethyl)-1H-pyrrole-2-carboxamide |
| A58 | 555 | N-(4-((2R)-1-(4-(1-acetylpiperidin-2-yl)phenyl)pyrrolidin-2-yl)thiazol-2-yl)-1-(pyridin-4- |
| | | ylmethyl)-1H-pyrrole-2-carboxamide |
| A63 | 555 | N-(4-((2R)-1-(4-(1-acetylpiperidin-3-yl)phenyl)pyrrolidin-2-yl)thiazol-2-yl)-1-(pyridin-4- |
| | | ylmethyl)-1H-pyrrole-2-carboxamide |
| C103 | 578 | (R)-N-(4-(1-(4-((1-acetylpiperidin-4-yl)oxy)phenyl)pyrrolidin-2-yl)thiazol-2-yl)-1- |
| | | ((tetrahydro-2H-pyran-4-yl)methyl)-1H-pyrrole-2-carboxamide |
| A62 | 589 | (R)-N-(4-(1-(4-((1-acetylpiperidin-4-yl)oxy)phenyl)pyrrolidin-2-yl)thiazol-2-yl)-1-((2- |
| | | fluoropyridin-4-yl)methyl)-1H-pyrrole-2-carboxamide |
| A90 | 607 | (R)-N-(4-(1-(4-((1-(2-fluoroacetyl)piperidin-4-yl)oxy)phenyl)pyrrolidin-2-yl)thiazol-2-yl)- |
| | | 1-((2-fluoropyridin-4-yl)methyl)-1H-pyrrole-2-carboxamide |
| A91 | 625 | (R)-N-(4-(1-(4-((1-(2,2-difluoroacetyl)piperidin-4-yl)oxy)phenyl)pyrrolidin-2-yl)thiazol- |
| | | 2-yl)-1-((2-fluoropyridin-4-yl)methyl)-1H-pyrrole-2-carboxamide |
| A92 | 643 | (R)-1-((2-fluoropyridin-4-yl)methyl)-N-(4-(1-(4-((1-(2,2,2-trifluoroacetyl)piperidin-4- |
| | | yl)oxy)phenyl)pyrrolidin-2-yl)thiazol-2-yl)-1H-pyrrole-2-carboxamide |
| A136 | 604 | (R)-4-(4-(2-(2-(1-((2-fluoropyridin-4-yl)methyl)-1H-pyrrole-2-carboxamido)thiazol-4- |
| | | yl)pyrrolidin-1-yl)phenoxy)-N-methylpiperidine-1-carboxamide |
| A137 | 618 | (R)-4-(4-(2-(2-(1-((2-fluoropyridin-4-yl)methyl)-1H-pyrrole-2-carboxamido)thiazol-4- |
| | | yl)pyrrolidin-1-yl)phenoxy)-N,N-dimethylpiperidine-1-carboxamide |
| Int. for A139 | 695 | (R)-N-(4-1-(4-((1-2-(benzyloxy)acetyl)piperidin-4-yl)oxy)phenyl)pyrrolidin-2- |
| | | yl)thiazol-2-yl)-1-((2-fluoropyridin-4-yl)methyl)-1H-pyrrole-2-carboxamide |
| A64 | 519 | (R)-N-(4-(1-(4-(acetamidomethyl)phenyl)pyrrolidin-2-yl)thiazol-2-yl)-1-((2- |
| | | fluoropyridin-4-yl)methyl)-1H-pyrrole-2-carboxamide |
| C104 | 508 | (R)-N-(4-(1-(4-(acetamidomethyl)phenyl)pyrrolidin-2-yl)thiazol-2-yl)-1-((tetrahydro-2H- |
| | | pyran-4-yl)methyl)-1H-pyrrole-2-carboxamide |
| A66 | 515 | N-(4-((R)-1-(4-((R)-1-acetamidoethyl)phenyl)pyrrolidin-2-yl)thiazol-2-yl)-1-(pyridin-4- |
| | | ylmethyl)-1H-pyrrole-2-carboxamide |
| A68 | 505 | (R)-N-(4-(1-(4-acetamidophenyl)pyrrolidin-2-yl)thiazol-2-yl)-1-((2-fluoropyridin-4- |
| | | yl)methyl)-1H-pyrrole-2-carboxamide |
| A119 | 520 | (R)-1-((2-fluoropyridin-4-yl)methyl)-N-(4-(1-(4-(3-methylureido)phenyl)pyrrolidin-2- |
| | | yl)thiazol-2-yl)-1H-pyrrole-2-carboxamide |
| A124 | 534 | (R)-N-(4-(1-(4-(3,3-dimethylureido)phenyl)pyrrolidin-2-yl)thiazol-2-yl)-1-((2- |
| | | fluoropyridin-4-yl)methyl)-1H-pyrrole-2-carboxamide |
| Int. for A141 | 611 | (R)-N-(4-(1-(4-(2-(benzyloxy)acetamido)phenyl)pyrrolidin-2-yl)thiazol-2-yl)-1-((2- |
| | | fluoropyridin-4-yl)methyl)-1H-pyrrole-2-carboxamide |
| A144 | 521 | methyl (R)-(4-(2-(2-(1-((2-fluoropyridin-4-yl)methyl)-1H-pyrrole-2- |
| | | carboxamido)thiazol-4-yl)pyrrolidin-1-yl)phenyl)carbamate |
| A145 | 535 | ethyl (R)-(4-(2-(2-(1-((2-fluoropyridin-4-yl)methyl)-1H-pyrrole-2-carboxamido)thiazol- |
| | | 4-yl)pyrrolidin-1-yl)phenyl)carbamate |
| A146 | 549 | isopropyl (R)-(4-(2-(2-(1-((2-fluoropyridin-4-yl)methyl)-1H-pyrrole-2- |
| | | carboxamido)thiazol-4-yl)pyrrolidin-1-yl)phenyl)carbamate |
| A171 | 577 | (R)-tetrahydrofuran-3-yl (4-((R)-2-(2-(1-((2-fluoropyridin-4-yl)methyl)-1H-pyrrole-2- |
| | | carboxamido)thiazol-4-yl)pyrrolidin-1-yl)phenyl)carbamate |
| Int. for A173, | 662 | tert-butyl (R)-3-(((4-(2-(2-(1-((2-fluoropyridin-4-yl)methyl)-1H-pyrrole-2- |
| A178, and A179 | | carboxamido)thiazol-4-yl)pyrrolidin-1-yl)phenyl)carbamoyl)oxy)azetidine-1- |
| | | carboxylate |
| A179 | 604 | 1-acetylazetidin-3-yl (R)-(4-(2-(2-(1-((2-fluoropyridin-4-yl)methyl)-1H-pyrrole-2- |
| | | carboxamido)thiazol-4-yl)pyrrolidin-1-yl)phenyl)carbamate |
| A174 | 577 | (S)-tetrahydrofuran-3-yl (4-((R)-2-(2-(1-((2-fluoropyridin-4-yl)methyl)-1H-pyrrole-2- |
| | | carboxamido)thiazol-4-yl)pyrrolidin-1-yl)phenyl)carbamate |
| A177 | 563 | oxetan-3-yl (R)-(4-(2-(2-(1-((2-fluoropyridin-4-yl)methyl)-1H-pyrrole-2- |
| | | carboxamido)thiazol-4-yl)pyrrolidin-1-yl)phenyl)carbamate |
| A181 | 519 | (R)-1-((2-fluoropyridin-4-yl)methyl)-N-(4-(1-(4-propionamidophenyl)pyrrolidin-2- |
| | | yl)thiazol-2-yl)-1H-pyrrole-2-carboxamide |
| A182 | 533 | (R)-1-((2-fluoropyridin-4-yl)methyl)-N-(4-(1-(4-isobutyramidophenyl)pyrrolidin-2- |
| | | yl)thiazol-2-yl)-1H-pyrrole-2-carboxamide |
| A183 | 547 | (R)-1-((2-fluoropyridin-4-yl)methyl)-N-(4-(1-(4-pivalamidophenyl)pyrrolidin-2- |
| | | yl)thiazol-2-yl)-1H-pyrrole-2-carboxamide |
| A184 | 531 | (R)-N-(4-(1-(4-(cyclopropanecarboxamido)phenyl)pyrrolidin-2-yl)thiazol-2-yl)-1-((2- |
| | | fluoropyridin-4-yl)methyl)-1H-pyrrole-2-carboxamide |
| C107 | 494 | (R)-N-(4-(1-(4-acetamidophenyl)pyrrolidin-2-yl)thiazol-2-yl)-1-((tetrahydro-2H-pyran- |
| | | 4-yl)methyl)-1H-pyrrole-2-carboxamide |
| A69 | 533 | N-(4-((R)-1-(4-((S)-1-acetamidoethyl)phenyl)pyrrolidin-2-yl)thiazol-2-yl)-1-((2- |
| | | fluoropyridin-4-yl)methyl)-1H-pyrrole-2-carboxamide |
| A70 | 533 | N-(4-((R)-1-(4-((R)-1-acetamidoethyl)phenyl)pyrrolidin-2-yl)thiazol-2-yl)-1-((2- |
| | | fluoropyridin-4-yl)methyl)-1H-pyrrole-2-carboxamide |
| A73 | 515 | N-(4-((R)-1-(4-((S)-1-acetamidoethyl)phenyl)pyrrolidin-2-yl)thiazol-2-yl)-1-(pyridin-4- |
| | | ylmethyl)-1H-pyrrole-2-carboxamide |
| A79 | 575 | N-(4-((2R)-1-(4-((1-acetylpyrrolidin-3-yl)oxy)phenyl)pyrrolidin-2-yl)thiazol-2-yl)-1-((2- |
| | | fluoropyridin-4-yl)methyl)-1H-pyrrole-2-carboxamide |
| A81 | 561 | (R)-N-(4-(1-(4-((1-acetylazetidin-3-yl)oxy)phenyl)pyrrolidin-2-yl)thiazol-2-yl)-1-((2- |
| | | fluoropyridin-4-yl)methyl)-1H-pyrrole-2-carboxamide |
| A109 | 587 | (R)-N-(4-(1-(4-((1-(cyclopropanecarbonyl)azetidin-3-yl)oxy)phenyl)pyrrolidin-2- |
| | | yl)thiazol-2-yl)-1-((2-fluoropyridin-4-yl)methyl)-1H-pyrrole-2-carboxamide |
| Int. for A110 | 667 | (R)-N-(4-(1-(4-((1-(2-(benzyloxy)acetyl)azetidin-3-yl)oxy)phenyl)pyrrolidin-2-yl)thiazol- |
| | | 2-yl)-1-((2-fluoropyridin-4-yl)methyl)-1H-pyrrole-2-carboxamide |
| A125 | 576 | (R)-1-((2-fluoropyridin-4-yl)methyl)-N-(4-(1-(4-((1-(methylcarbamoyl)azetidin-3- |
| | | yl)oxy)phenyl)pyrrolidin-2-yl)thiazol-2-yl)-1H-pyrrole-2-carboxamide |
| A131 | 590 | (R)-N-(4-(1-(4-((1-(dimethylcarbamoyl)azetidin-3-yl)oxy)phenyl)pyrrolidin-2-yl)thiazol- |
| | | 2-yl)-1-((2-fluoropyridin-4-yl)methyl)-1H-pyrrole-2-carboxamide |
| A82 | 587 | (R)-N-(4-(1-(4-((1-acetyl-1,2,3,6-tetrahydropyridin-4-yl)oxy)phenyl)pyrrolidin-2- |
| | | yl)thiazol-2-yl)-1-((2-fluoropyridin-4-yl)methyl)-1H-pyrrole-2-carboxamide |
| A83 | 589 | N-(4-(R)-1-(4-(((R)-1-acetylpiperidin-3-yl)oxy)phenyl)pyrrolidin-2-yl)thiazol-2-yl)-1- |
| | | ((2-fluoropyridin-4-yl)methyl)-1H-pyrrole-2-carboxamide |
| A84 | 589 | N-(4-((R)-1-(4-(((S)-1-acetylpiperidin-3-yl)oxy)phenyl)pyrrolidin-2-yl)thiazol-2-yl)-1-((2- |
| | | fluoropyridin-4-yl)methyl)-1H-pyrrole-2-carboxamide |
| A93 | 523 | (R)-N-(4-(1-(4-acetamido-3-fluorophenyl)pyrrolidin-2-yl)thiazol-2-yl)-1-((2- |
| | | fluoropyridin-4-yl)methyl)-1H-pyrrole-2-carboxamide |
| A96 | 541 | (R)-N-(4-(1-(4-acetamido-2,6-difluorophenyl)pyrrolidin-2-yl)thiazol-2-yl)-1-((2- |
| | | fluoropyridin-4-yl)methyl)-1H-pyrrole-2-carboxamide |
| A97 | 603 | (R)-N-(4-(1-(4-((1-acetyl-4-methylpiperidin-4-yl)oxy)phenyl)pyrrolidin-2-yl)thiazol-2- |
| | | yl)-1-((2-fluoropyridin-4-yl)methyl)-1H-pyrrole-2-carboxamide |
| A98 | 523 | (R)-N-(4-(1-(4-acetamido-2-fluorophenyl)pyrrolidin-2-yl)thiazol-2-yl)-1-((2- |
| | | fluoropyridin-4-yl)methyl)-1H-pyrrole-2-carboxamide |
| A99 | 541 | (R)-N-(4-(1-(4-acetamido-3,5-difluorophenyl)pyrrolidin-2-yl)thiazol-2-yl)-1-((2- |
| | | fluoropyridin-4-yl)methyl)-1H-pyrrole-2-carboxamide |
| A100 | 607 | (R)-N-(4-(1-(4-((1-acetylpiperidin-4-yl)oxy)-2-fluorophenyl)pyrrolidin-2-yl)thiazol-2-yl)- |
| | | 1-((2-fluoropyridin-4-yl)methyl)-1H-pyrrole-2-carboxamide |
| A121 | 549 | (R)-N-(4-(1-(4-(2-acetamidoethoxy)phenyl)pyrrolidin-2-yl)thiazol-2-yl)-1-((2- |
| | | fluoropyridin-4-yl)methyl)-1H-pyrrole-2-carboxamide |
| A102 | 607 | (R)-N-(4-(1-(4-((1-acetylpiperidin-4-yl)oxy)-3-fluorophenyl)pyrrolidin-2-yl)thiazol-2-yl)- |
| | | 1-((2-fluoropyridin-4-yl)methyl)-1H-pyrrole-2-carboxamide |
| A117 | 563 | (R)-1-((2-fluoropyridin-4-yl)methyl)-N-(4-(1-(4-(2-(N- |
| | | methylacetamido)ethoxy)phenyl)pyrrolidin-2-yl)thiazol-2-yl)-1H-pyrrole-2- |
| | | carboxamide |
| A105 | 625 | (R)-N-(4-(1-(4-((1-acetylpiperidin-4-yl)oxy)-2,6-difluorophenyl)pyrrolidin-2-yl)thiazol-2- |
| | | yl)-1-((2-fluoropyridin-4-yl)methyl)-1H-pyrrole-2-carboxamide |
| A106 | 625 | (R)-N-(4-(1-(4-((1-acetylpiperidin-4-yl)oxy)-3,5-difluorophenyl)pyrrolidin-2-yl)thiazol-2- |
| | | yl)-1-((2-fluoropyridin-4-yl)methyl)-1H-pyrrole-2-carboxamide |
| A108 | 607 | N-(4-((2R)-1-(4-((1-acetyl-3-fluoropiperidin-4-yl)oxy)phenyl)pyrrolidin-2-yl)thiazol-2- |
| | | yl)-1-((2-fluoropyridin-4-yl)methyl)-1H-pyrrole-2-carboxamide |
| A122 | 523 | (R)-N-(4-(1-(4-acetamidophenyl)pyrrolidin-2-yl)thiazol-2-yl)-1-((2,6-difluoropyridin-4- |
| | | yl)methyl)-1H-pyrrole-2-carboxamide |
| A126 | 614 | (R)-N-(4-(1-(4-((1-acetylpiperidin-4-yl)oxy)-3-cyanophenyl)pyrrolidin-2-yl)thiazol-2-yl)- |
| | | 1-((2-fluoropyridin-4-yl)methyl)-1H-pyrrole-2-carboxamide |
| A129 | 619 | (R)-N-(4-(1-(4-((1-acetylpiperidin-4-yl)oxy)-3-methoxyphenyl)pyrrolidin-2-yl)thiazol-2- |
| | | yl)-1-((2-fluoropyridin-4-yl)methyl)-1H-pyrrole-2-carboxamide |
| A133 | 614 | (R)-N-(4-(1-(4-((1-acetylpiperidin-4-yl)oxy)-2-cyanophenyl)pyrrolidin-2-yl)thiazol-2-yl)- |
| | | 1-((2-fluoropyridin-4-yl)methyl)-1H-pyrrole-2-carboxamide |
| A140 | 623 | (R)-N-(4-(1-(4-((1-acetylpiperidin-4-yl)oxy)-3-chlorophenyl)pyrrolidin-2-yl)thiazol-2-yl)- |
| | | 1-((2-fluoropyridin-4-yl)methyl)-1H-pyrrole-2-carboxamide |
| C117 | 477 | N-(4-((R)-1-(4-acetamidophenyl)pyrrolidin-2-yl)thiazol-2-yl)-1-((S)-3-cyano-2- |
| | | methylpropyl)-1H-pyrrole-2-carboxamide |
| A143 | 623 | (R)-N-(4-(1-(4-((1-acetylpiperidin-4-yl)oxy)-2-chlorophenyl)pyrrolidin-2-yl)thiazol-2-yl)- |
| | | 1-((2-fluoropyridin-4-yl)methyl)-1H-pyrrole-2-carboxamide |
| A154 | 590 | (R)-N-(4-(1-(5-((1-acetylpiperidin-4-yl)oxy)pyridin-2-yl)pyrrolidin-2-yl)thiazol-2-yl)-1- |
| | | ((2-fluoropyridin-4-yl)methyl)-1H-pyrrole-2-carboxamide |
| A155 | 590 | (R)-N-(4-(1-(6-((1-acetylpiperidin-4-yl)oxy)pyridin-3-yl)pyrrolidin-2-yl)thiazol-2-yl)-1- |
| | | ((2-fluoropyridin-4-yl)methyl)-1H-pyrrole-2-carboxamide |
| A160 | 587 | (R)-N-(4-(1-(4-((1-acetylpiperidin-4-yl)methyl)phenyl)pyrrolidin-2-yl)thiazol-2-yl)-1-((2- |
| | | fluoropyridin-4-yl)methyl)-1H-pyrrole-2-carboxamide |
| A161 | 601 | N-(4-((2R)-1-(4-(1-(1-acetylpiperidin-4-yl)ethyl)phenyl)pyrrolidin-2-yl)thiazol-2-yl)-1- |
| | | ((2-fluoropyridin-4-yl)methyl)-1H-pyrrole-2-carboxamide |
| A163 | 548 | (R)-N-(4-(1-(4-(3,3-dimethylureido)phenyl)pyrrolidin-2-yl)thiazol-2-yl)-1-((2- |
| | | fluoropyridin-4-yl)methyl)-4-methyl-1H-pyrrole-2-carboxamide |
| A164 | 519 | (R)-N-(4-(1-(4-acetamidophenyl)pyrrolidin-2-yl)thiazol-2-yl)-1-((2-fluoropyridin-4- |
| | | yl)methyl)-4-methyl-1H-pyrrole-2-carboxamide |
| A172 | 535 | methyl (R)-(4-(2-(2-(1-((2-fluoropyridin-4-yl)methyl)-4-methyl-1H-pyrrole-2- |
| | | carboxamido)thiazol-4-yl)pyrrolidin-1-yl)phenyl)carbamate |
| A165 | 603 | (R)-N-(4-(1-(4-((1-acetylpiperidin-4-yl)oxy)phenyl)pyrrolidin-2-yl)thiazol-2-yl)-1-((2- |
| | | fluoropyridin-4-yl)methyl)-4-methyl-1H-pyrrole-2-carboxamide |
| Int. for A176 | 569 | (R)-N-(4-(1-(4-((1-acetylazetidin-3-yl)ethynyl)phenyl)pyrrolidin-2-yl)thiazol-2-yl)-1-((2- |
| | | fluoropyridin-4-yl)methyl)-1H-pyrrole-2-carboxamide |
| A175 | 522 | methyl (R)-(6-(2-(2-(1-((2-fluoropyridin-4-yl)methyl)-1H-pyrrole-2- |
| | | carboxamido)thiazol-4-yl)pyrrolidin-1-yl)pyridin-3-yl)carbamate |
| A180 | 536 | methyl (R)-(6-(2-(2-(1-((2-fluoropyridin-4-yl)methyl)-4-methyl-1H-pyrrole-2- |
| | | carboxamido)thiazol-4-yl)pyrrolidin-1-yl)pyridin-3-yl)carbamate |
| A186 | 547 | (R)-1-((2-fluoropyridin-4-yl)methyl)-N-(4-(1-(4-(oxetane-3- |
| | | carboxamido)phenyl)pyrrolidin-2-yl)thiazol-2-yl)-1H-pyrrole-2-carboxamide |
| A187 | 574 | (R)-N-(4-(2-(2-(1-((2-fluoropyridin-4-yl)methyl)-1H-pyrrole-2-carboxamido)thiazol-4- |
| | | yl)pyrrolidin-1-yl)phenyl)piperidine-1-carboxamide |
| A188 | 576 | (R)-N-(4-(2-(2-(1-((2-fluoropyridin-4-yl)methyl)-1H-pyrrole-2-carboxamido)thiazol-4- |
| | | yl)pyrrolidin-1-yl)phenyl)morpholine-4-carboxamide |
| A190 | 561 | 1-((2-fluoropyridin-4-yl)methyl)-N-(4-((2R)-1-(4-(tetrahydrofuran-3- |
| | | carboxamido)phenyl)pyrrolidin-2-yl)thiazol-2-yl)-1H-pyrrole-2-carboxamide |

### Route 4:

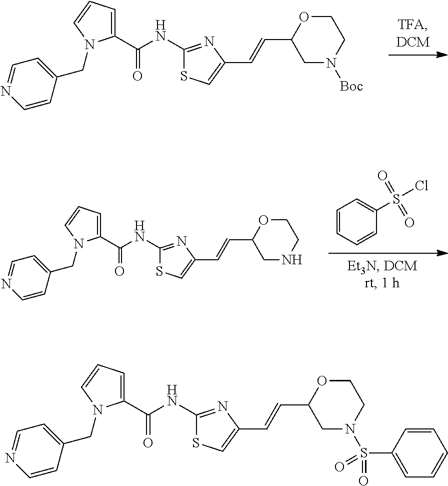

The Boc group was removed as described in route 1.

A 50 mL vial with stir bar was charged with N-[4-[(E)-2-(morpholin-2-yl)ethenyl]-1,3-thiazol-2-yl]-1-(pyridin-4-ylmethyl)pyrrole-2-carboxamide (100.00 mg, 0.25 mmol, 1.00 equiv), Et₃N (75.76 mg, 0.75 mmol, 3.00 equiv) and DCM (5.00 mL), benzenesulfonyl chloride (53.00 mg, 0.30 mmol, 1.20 equiv) was added at 0° C. The vial was capped and placed in a room temperature bath. The reaction mixture was stirred at room temperature for 2 h. The reaction mixture was poured into DCM (20 mL) and washed with brine (1×20 mL). The organic layer was then dried over Na₂SO₄, filtered and concentrated in vacuo. The resulting crude material was purified via RP column to yield the desired product.

| | Observed | |
|---|---|---|
| | molecular | |
| | ion | Compound name |
| B9 | 536 | (E)-N-(4-(2-(4-(phenylsulfonyl)morpholin-2-yl)vinyl)thiazol-2-yl)-1-(pyridin-4- |
| | | ylmethyl)-1H-pyrrole-2-carboxamide |
| A128 | 625 | (R)-1-((2-fluoropyridin-4-yl)methyl)-N-(4-(1-(4-((1-(methylsulfonyl)piperidin-4- |
| | | yl)oxy)phenyl)pyrrolidin-2-yl)thiazol-2-yl)-1H-pyrrole-2-carboxamide |
| A118 | 541 | (R)-1-((2-fluoropyridin-4-yl)methyl)-N-(4-(1-(4- |
| | | (methylsulfonamido)phenyl)pyrrolidin-2-yl)thiazol-2-yl)-1H-pyrrole-2-carboxamide |
| A132 | 597 | (R)-1-((2-fluoropyridin-4-yl)methyl)-N-(4-(1-(4-((1-(methylsulfonyl)azetidin-3- |
| | | yl)oxy)phenyl)pyrrolidin-2-yl)thiazol-2-yl)-1H-pyrrole-2-carboxamide |
| A148 | 626 | (R)-N-(4-(1-(4-((1-(N,N-dimethylsulfamoyl)azetidin-3-yl)oxy)phenyl)pyrrolidin-2- |
| | | yl)thiazol-2-yl)-1-((2-fluoropyridin-4-yl)methyl)-1H-pyrrole-2-carboxamide |
| Int. for A150 | 698 | tert-butyl (R)-((3-(4-(2-(2-(1-((2-fluoropyridin-4-yl)methyl)-1H-pyrrole-2- |
| | | carboxamido)thiazol-4-yl)pyrrolidin-1-yl)phenoxy)azetidin-1-yl)sulfonyl)carbamate |
| A151 | 612 | (R)-1-((2-fluoropyridin-4-yl)methyl)-N-(4-(1-(4-((1-(N-methylsulfamoyl)azetidin-3- |
| | | yl)oxy)phenyl)pyrrolidin-2-yl)thiazol-2-yl)-1H-pyrrole-2-carboxamide |
| A153 | 581 | 1-((2-fluoropyridin-4-yl)methyl)-N-(4-((2R)-1-(4-((1-(methylsulfinyl)azetidin-3- |
| | | yl)oxy)phenyl)pyrrolidin-2-yl)thiazol-2-yl)-1H-pyrrole-2-carboxamide |

### Route 5:

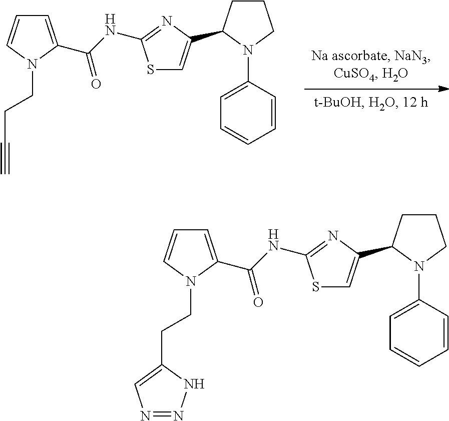 [SMILES: C#CCCn1cccc1C(=O)Nc1nc([C@H]2CCCN2c2ccccc2)cs1 ; "Na ascorbate, NaN3,CuSO4, H2O" ; "t-BuOH, H2O, 12 h" ; O=C(Nc1nc([C@H]2CCCN2c2ccccc2)cs1)c1cccn1CCc1cnn[nH]1]

A 100 mL vial with stir bar was charged with 1-(but-3-yn-1-yl)-N-[4-[(2R)-1-phenylpyrrolidin-2-yl]-1,3-thiazol-2-yl]pyrrole-2-carboxamide (150.00 mg, 0.38 mmol, 1.00 equiv), sodium ascorbate (15.30 mg, 0.08 mmol, 0.20 equiv), NaN₃ (49.94 mg, 0.77 mmol, 2.00 equiv), CuSO₄.5H₂O (20.00 mg, 0.08 mmol, 0.20 equiv), t-BuOH (4 ml) and H₂ (4 mL). The vial was capped and placed in a 8000 bath. The reaction mixture was stirred at 8000 overnight. The next morning, the reaction mixture was cooled to room temperature and concentrated under vacuum. The resulting crude material was purified via RP column to yield the desired product.

The following compounds were prepared via a similar method:

| | Observed | |
|---|---|---|
| | molecular | |
| | ion | Compound name |
| C29 | 434 | (R)-1-(2-(1H-1,2,3-triazol-5-yl)ethyl)-N-(4-(1-phenylpyrrolidin-2- |
| | | yl)thiazol-2-yl)-1H-pyrrole-2-carboxamide |
| C27 | 448 | (R)-1-(3-(1H-1,2,3-triazol-5-yl)propyl)-N-(4-(1-phenylpyrrolidin-2- |
| | | yl)thiazol-2-yl)-1H-pyrrole-2-carboxamide |
| C26 | 462 | (R)-1-(4-(1H-1,2,3-triazol-5-yl)butyl)-N-(4-(1-phenylpyrrolidin-2- |
| | | yl)thiazol-2-yl)-1H-pyrrole-2-carboxamide |
| C29 | 420 | (R)-1-((1H-1,2,3-triazol-5-yl)methyl)-N-(4-(1-phenylpyrrolidin-2- |
| | | yl)thiazol-2-yl)-1H-pyrrole-2-carboxamide |

### Route 6:

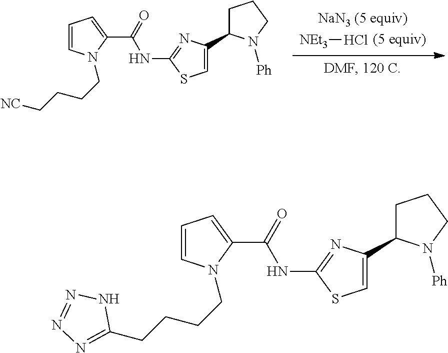

CAUTION! A vial with stir bar was charged with nitrile (30 mg, 0.072 mmol, 1.0 equiv), triethylamine hydrochloride (49 mg, 0.36 mmol, 5.0 equiv) and sodium azide (23 mg, 0.36 mmol, 5.0 equiv). DMF (0.3 mL) was added, and the reaction mixture was stirred at 120 C overnight. The next morning, the reaction mixture was cooled to room temperature and quenched with a few drops of brine. The reaction mixture was poured into 10% MeOH in DCM (1×50 mL) and washed with brine (2×50 mL). The combined aqueous layers were extracted with 10% MeOH in DCM (1×50 mL) and the azide-containing aqueous layer was quenched with sodium nitrite followed by sulfuric acid until bubbling stopped. The combined organic layers were dried over Na₂SO₄, filtered and concentrated in vacuo. The resulting crude material was purified via silica gel chromatography to yield the desired product.

The following compounds were prepared via a similar method:

| | Observed | |
|---|---|---|
| | molecular | |
| | ion | Compound name |
| C4 | 463 | (R)-1-(4-(1H-tetrazol-5-yl)butyl)-N-(4-(1-phenylpyrrolidin- |
| | | 2-yl)thiazol-2-yl)-1H-pyrrole-2-carboxamide |
| C14 | 449 | (R)-1-(3-(1H-tetrazol-5-yl)propyl)-N-(4-(1-phenylpyrrolidin- |
| | | 2-yl)thiazol-2-yl)-1H-pyrrole-2-carboxamide |
| C16 | 435 | (R)-1-(2-(1H-tetrazol-5-yl)ethyl)-N-(4-(1-phenylpyrrolidin- |
| | | 2-yl)thiazol-2-yl)-1H-pyrrole-2-carboxamide |

### Route 7:

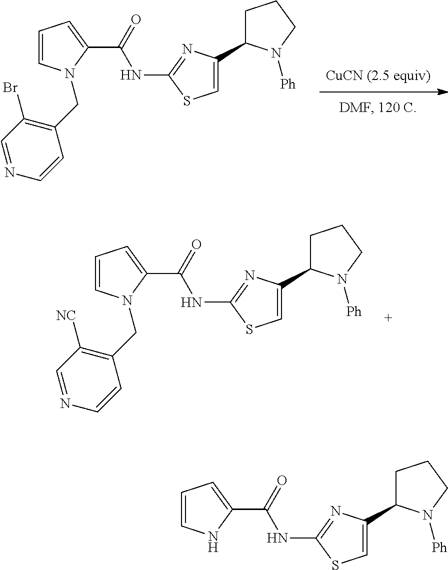 [SMILES: O=C(Nc1nc([C@H]2CCCN2c2ccccc2)cs1)c1cccn1Cc1ccncc1Br ; N#Cc1cnccc1Cn1cccc1C(=O)Nc1nc([C@H]2CCCN2c2ccccc2)cs1 ; O=C(Nc1nc([C@H]2CCCN2c2ccccc2)cs1)c1ccc[nH]1]

A vial with stir bar was charged with bromide (50 mg, 0.098 mmol, 1.0 equiv) and CuCN (22 mg, 0.25 mmol, 2.5 equiv). DMF (0.4 mL) was added, and the reaction mixture was allowed to stir at room temperature overnight. The next morning, the reaction mixture was cooled to room temperature and diluted with EtOAc (50 mL). The organic layer was washed with saturated NaHCO₃ (2×50 mL), and the combined aqueous layers were extracted with EtOAc (1×50 mL). The combined organic layers were dried over Na2SO4, filtered and concentrated in vacuo. The resulting crude material was purified via silica gel chromatography to yield the desired products.

The following compounds were prepared via a similar method:

| | Observed | |
|---|---|---|
| | molecular | |
| | ion | Compound name |
| E59 | 455 | (R)-1-((3-cyanopyridin-4-yl)methyl)-N-(4-(1-phenylpyrrolidin-2-yl)thiazol- |
| | | 2-yl)-1H-pyrrole-2-carboxamide |
| E58 | 339 | (R)-N-(4-(1-phenylpyrrolidin-2-yl)thiazol-2-yl)-1H-pyrrole-2-carboxamide |
| E60 | 455 | (R)-1-((2-cyanopyridin-4-yl)methyl)-N-(4-(1-phenylpyrrolidin-2-yl)thiazol- |
| | | 2-yl)-1H-pyrrole-2-carboxamide |

### Route 8:

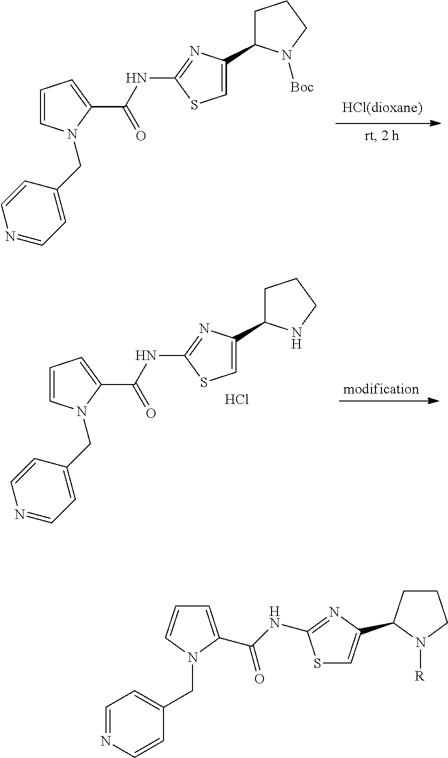

A 100 mL vial with stir bar was charged with tert-butyl (2R)-2-[2-[1-(pyridin-4-ylmethyl)pyrrole-2-amido]-1,3-thiazol-4-yl]pyrrolidine-1-carboxylate (2.00 g, 4.41 mmol, 1.00 equiv) and HCl (dioxane) (4M, 15.00 mL) and dioxane (10 mL). The vial was capped and placed in a room temperature bath. The reaction mixture was stirred at room temperature for 2 h. The solids were collected by filtration and concentrated in vacuo. The resulting crude material was used directly for next step.

Modification 1: SNAr

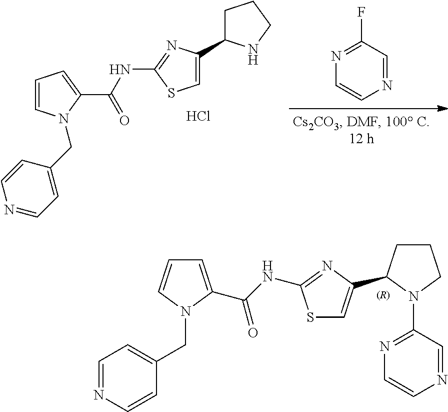

A 100 mL vial with stir bar was charged with 1-(pyridin-4-ylmethyl)-N-[4-[(2R)-pyrrolidin-2-yl]-1,3-thiazol-2-yl]pyrrole-2-carboxamide (200.00 mg, 0.57 mmol, 1.00 equiv), Cs₂CO₃ (924.70 mg, 2.83 mmol, 5.00 equiv), 2-fluoropyrazine (66.60 mg, 0.68 mmol, 1.20 equiv) and DMF (20.00 mL) under nitrogen atmosphere. The vial was capped and placed in a 100° C. bath. The reaction mixture was stirred at 100° C. for 4 h. The reaction mixture was cooled to room temperature and concentrated under vacuum. The reaction mixture was then quenched by H₂O (80 mL). The resulting solution was extracted with ethyl acetate (3×80 mL) and washed with brine (3×80 mL), and the organic layers were dried over Na₂SO₄, filtered and concentrated in vacuo. The resulting crude material was purified via silica gel chromatography & RP column to yield the desired product.

Modification 2: Reductive Amination

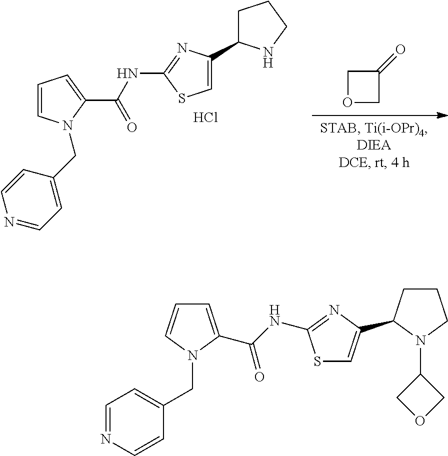

A 50 mL vial with stir bar was charged with 1-(pyridin-4-ylmethyl)-N-[4-[(2R)-pyrrolidin-2-yl]-1,3-thiazol-2-yl]pyrrole-2-carboxamide hydrochloride (100.00 mg, 0.26 mmol, 1.00 equiv), 3-oxetanone (22.18 mg, 0.31 mmol, 1.20 equiv), DIEA (33.15 mg, 0.26 mmol, 1.00 equiv) and DCE (10.00 mL), STAB (110.00 mg, 0.52 mmol, 2.00 equiv) under nitrogen atmosphere, Ti(Oi-Pr)₄ (147.00 mg, 0.52 mmol, 2.00 equiv) was added. The vial was capped and placed in a room temperature bath. The reaction mixture was stirred at room temperature for 4 h. The reaction mixture was then quenched by H₂O (20 mL). The resulting solution was extracted with ethyl acetate (3×30 mL) and washed with brine (1×30 mL), and the organic layers were dried over Na₂SO₄, filtered and concentrated in vacuo. The resulting crude material was purified via silica gel chromatography & prep-HPLC column to yield the desired product.

Modification 3: Sulfonylation

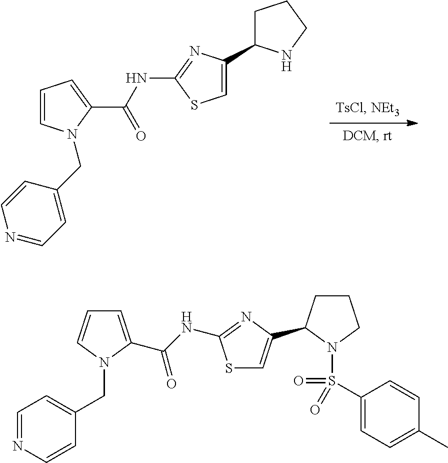

A vial with stir bar was charged with amine (47 mg, 0.13 mmol, 1.0 equiv), TsCl (30 mg, 0.16 mmol, 1.2 equiv) and DCM (1 mL). Triethylamine (37 uL, 0.27 mmol, 2.0 equiv) was added, and the reaction mixture was allowed to stir at room temperature overnight. The next morning, the mixture was diluted with DCM (50 mL) and washed with brine (2×50 mL). The combined aqueous layers were extracted with DCM (1×50 mL). The combined organic layers were dried over Na₂SO₄, filtered and concentrated in vacuo. The resulting crude material was purified via silica gel chromatography to yield the desired product.

The following compounds were prepared via a similar method:

| | Modifi- | Observed | |
|---|---|---|---|
| | cation | molecular ion | Compound name |
| A30 | 1 | 432 | (R)-N-(4-(1-(pyrazin-2-yl)pyrrolidin-2-yl)thiazol-2-yl)-1-(pyridin-4- |
| | | | ylmethyl)-1H-pyrrole-2-carboxamide |
| E48 | 1 | 431 | (R)-N-(4-(1-(pyridin-4-yl)pyrrolidin-2-yl)thiazol-2-yl)-1-(pyridin-4- |
| | | | ylmethyl)-1H-pyrrole-2-carboxamide |
| E49 | 2 | 410 | (R)-N-(4-(1-(oxetan-3-yl)pyrrolidin-2-yl)thiazol-2-yl)-1-(pyridin-4- |
| | | | ylmethyl)-1H-pyrrole-2-carboxamide |
| E14 | 3 | 508 | (R)-1-(pyridin-4-ylmethyl)-N-(4-(1-tosylpyrrolidin-2-yl)thiazol-2- |
| | | | yl)-1H-pyrrole-2-carboxamide |
| E52 | 3 | 662 | (R)-1-(pyridin-4-ylmethyl)-N-tosyl-N-(4-(1-tosylpyrrolidin-2- |
| | | | yl)thiazol-2-yl)-1H-pyrrole-2-carboxamide |
| A12 and Ints for | 1 | 488 | methyl (R)-4-(2-(2-(1-(pyridin-4-ylmethyl)-1H-pyrrole-2- |
| A13, A17 and | | | carboxamido)thiazol-4-yl)pyrrolidin-1-yl)benzoate |
| A18 | | | |
| A27 and Ints for | 1 | 475 | (R)-N-(4-(1-(4-nitrophenyl)pyrrolidin-2-yl)thiazol-2-yl)-1-(pyridin- |
| A15, A16 and | | | 4-ylmethyl)-1H-pyrrole-2-carboxamide |
| A20 | | | |
| Int. for A19 | 1 | 455 | (R)-N-(4-(1-(4-cyanophenyl)pyrrolidin-2-yl)thiazol-2-yl)-1- |
| | | | (pyridin-4-ylmethyl)-1H-pyrrole-2-carboxamide |
| A32 | 1 | 432 | (R)-1-(pyridin-4-ylmethyl)-N-(4-(1-(pyrimidin-2-yl)pyrrolidin-2- |
| | | | yl)thiazol-2-yl)-1H-pyrrole-2-carboxamide |
| A29 | 1 | 432 | (R)-N-(4-(1-(pyridazin-3-yl)pyrrolidin-2-yl)thiazol-2-yl)-1-(pyridin- |
| | | | 4-ylmethyl)-1H-pyrrole-2-carboxamide |
| E17 | None | 354 | (R)-1-(pyridin-4-ylmethyl)-N-(4-(pyrrolidin-2-yl)thiazol-2-yl)-1H- |
| | | | pyrrole-2-carboxamide |

### Route 9:

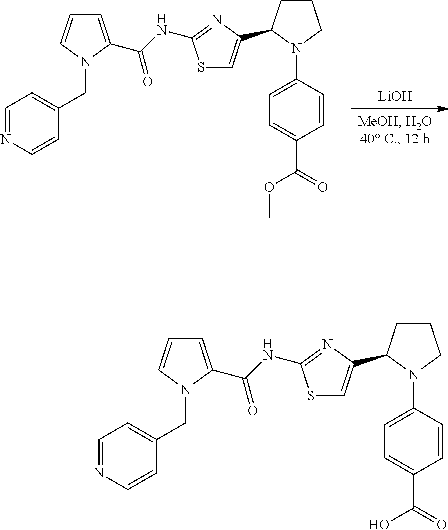 [SMILES: COC(=O)c1ccc(N2CCC[C@@H]2c2csc(NC(=O)c3cccn3Cc3ccncc3)n2)cc1 ; "LiOH" ; "MeOH, H2O40° C., 12 h" ; O=C(O)c1ccc(N2CCC[C@@H]2c2csc(NC(=O)c3cccn3Cc3ccncc3)n2)cc1]

A 50 mL vial with stir bar was charged with methyl 4-[(2R)-2-[2-[1-(pyridin-4-ylmethyl)pyrrole-2-amido]-1,3-thiazol-4-yl]pyrrolidin-1-yl]benzoate (50.00 mg, 0.10 mmol, 1.00 equiv), LiOH (12.28 mg, 0.51 mmol, 5.00 equiv), MeOH (3.00 mL) and H₂O (1.00 mL). The vial was capped and placed in a 40° C. bath. The reaction mixture was stirred at 40° C. overnight. The next morning, the pH value of the solution was adjusted to 7 with HCl(aq) (1 M). The resulting solution was extracted with dichloromethane (3×30 mL) and the organic layers were dried over Na₂SO₄, filtered and concentrated in vacuo. The resulting crude material was purified via RP column to yield the desired product.

| | Observed | |
|---|---|---|
| | mass | Compound name |
| A13 | 474 | (R)-4-(2-(2-(1-(pyridin-4-ylmethyl)-1H-pyrrole-2-carboxamido)thiazol-4- |
| | | yl)pyrrolidin-1-yl)benzoic acid |

### Route 10:

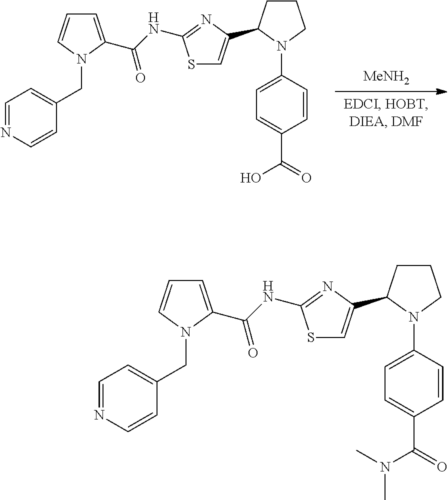 [SMILES: "MeNH2" ; O=C(O)c1ccc(N2CCC[C@@H]2c2csc(NC(=O)c3cccn3Cc3ccncc3)n2)cc1 ; "EDCI, HOBT,DIEA, DMF" ; CN(C)C(=O)c1ccc(N2CCC[C@@H]2c2csc(NC(=O)c3cccn3Cc3ccncc3)n2)cc1]

A 50 mL vial with stir bar was charged with 4-[(2R)-2-[2-[1-(pyridin-4-ylmethyl)pyrrole-2-amido]-1,3-thiazol-4-yl]pyrrolidin-1-yl]benzoic acid (50.00 mg, 0.11 mmol, 1.00 equiv), EDCI (30.36 mg, 0.16 mmol, 1.50 equiv), HOBT (21.40 mg, 0.16 mmol, 1.50 equiv), DIEA (27.29 mg, 0.21 mmol, 2.00 equiv) and DMF (3.00 mL), the reaction mixture was stirred 20 min, and then methylamine (6.83 mg, 0.22 mmol, 2.00 equiv) was added. The vial was capped and placed in a room temperature bath. The reaction mixture was stirred at room temperature for 2 h. The reaction was then quenched by H₂O (20 mL). The resulting solution was extracted with EtOAc (3×20 mL) and washed with brine (3×20 mL). The combined organic layers were dried over Na₂SO₄, filtered and concentrated in vacuo. The resulting crude material was purified via silica gel chromatography & RP column to yield the desired product.

The following compounds were prepared via a similar method:

| | Observed | |
|---|---|---|
| | molecular | |
| | ion | Compound name |
| A18 | 501 | (R)-N-(4-(1-(4-(dimethylcarbamoyl)phenyl)pyrrolidin-2-yl)thiazol-2-yl)-1-(pyridin-4- |
| | | ylmethyl)-1H-pyrrole-2-carboxamide |
| A17 | 487 | (R)-N-(4-(1-(4-(methylcarbamoyl)phenyl)pyrrolidin-2-yl)thiazol-2-yl)-1-(pyridin-4- |
| | | ylmethyl)-1H-pyrrole-2-carboxamide |
| C60 | 466 | (R)-1-(5-(dimethylamino)-5-oxopentyl)-N-(4-(1-phenylpyrrolidin-2-yl)thiazol-2-yl)-1H- |
| | | pyrrole-2-carboxamide |
| C59 | 438 | (R)-1-(3-(dimethylamino)-3-oxopropyl)-N-(4-(1-phenylpyrrolidin-2-yl)thiazol-2-yl)-1H- |
| | | pyrrole-2-carboxamide |
| C61 | 452 | (R)-1-(4-(dimethylamino)-4-oxobutyl)-N-(4-(1-phenylpyrrolidin-2-yl)thiazol-2-yl)-1H- |
| | | pyrrole-2-carboxamide |

### Route 11:

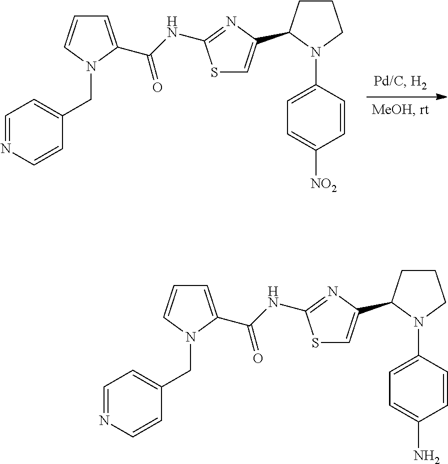 [SMILES: O=C(Nc1nc([C@H]2CCCN2c2ccc([N+](=O)[O-])cc2)cs1)c1cccn1Cc1ccncc1 ; "MeOH, rt" ; "Pd/C, H2" ; Nc1ccc(N2CCC[C@@H]2c2csc(NC(=O)c3cccn3Cc3ccncc3)n2)cc1]

A 50 mL vial with stir bar was charged with N-[4-[(2R)-1-(4-nitrophenyl)pyrrolidin-2-yl]-1,3-thiazol-2-yl]-1-(pyridin-4-ylmethyl)pyrrole-2-carboxamide (120.00 mg, 0.25 mmol, 1.00 equiv), Pd/C (10%, 53.2 mg, 0.50 mmol, 2.00 equiv) in MeOH (10 mL) under nitrogen atmosphere. The flask was then vacuumed and flushed with hydrogen. The reaction mixture was hydrogenated at room temperature for 2 hours under hydrogen atmosphere using a hydrogen balloon. Then the reaction mixture was filtered through a celite pad and the filtrate was concentrated under reduced pressure. The resulting crude material was purified via RP column to yield the desired product.

The following compounds were prepared via a similar method:

| | Observed | |
|---|---|---|
| | molecular | |
| | ion | Compound name |
| A20 | 445 | (R)-N-(4-(1-(4-aminophenyl)pyrrolidin-2-yl)thiazol-2-yl)-1-(pyridin-4- |
| | | ylmethyl)-1H-pyrrole-2-carboxamide |
| E4 | 394 | N-(4-(2-(2-methyloxazol-4-yl)ethyl)thiazol-2-yl)-1-(pyridin-4-ylmethyl)-1H- |
| | | pyrrole-2-carboxamide |

### Route 12:

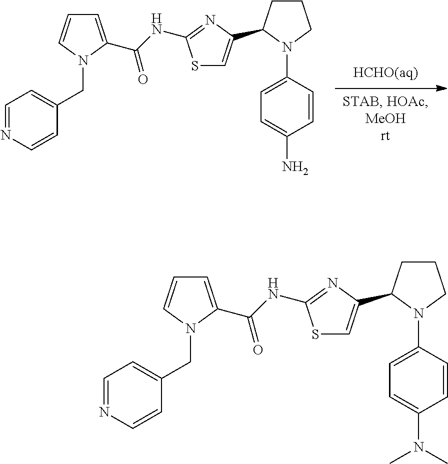 [SMILES: Nc1ccc(N2CCC[C@@H]2c2csc(NC(=O)c3cccn3Cc3ccncc3)n2)cc1 ; "HCHO(aq)" ; "STAB, HOAc,MeOHrt" ; CN(C)c1ccc(N2CCC[C@@H]2c2csc(NC(=O)c3cccn3Cc3ccncc3)n2)cc1]

A 100 mL vial with stir bar was charged with N-[4-[(2R)-1-(4-aminophenyl)pyrrolidin-2-yl]-1,3-thiazol-2-yl]-1-(pyridin-4-ylmethyl)pyrrole-2-carboxamide (120.00 mg, 0.27 mmol, 1.00 equiv), HCHO (aq) (37%, 65.68 mg, 0.81 mmol, 3.00 equiv), AcOH (8.10 mg, 0.14 mmol, 0.50 equiv) and MeOH (8 mL), STAB (200.23 mg, 0.95 mmol, 3.50 equiv) was added. The vial was capped and placed in a room temperature bath. The reaction mixture was stirred at room temperature for 2 h. The pH value of the solution was adjusted to 7 with NaHCO₃ (aq). The resulting solution was extracted with (3×30 mL) of ethyl acetate and washed with brine (1×20 mL). The combined organic layers were dried over Na₂SO₄, filtered and concentrated in vacuo. The resulting crude material was purified via prep-HPLC column to yield the desired product.

The following compounds were prepared via a similar method:

| | Observed | |
|---|---|---|
| | molecular | |
| | ion | Compound name |
| A16 | 473 | (R)-N-(4-(1-(4-(dimethylamino)phenyl)pyrrolidin-2-yl)thiazol-2-yl)-1-(pyridin-4- |
| | | ylmethyl)-1H-pyrrole-2-carboxamide |
| A147 | 588 | (R)-N-(4-(1-(4-((1-acetylpiperidin-4-yl)amino)phenyl)pyrrolidin-2-yl)thiazol-2-yl)-1- |
| | | ((2-fluoropyridin-4-yl)methyl)-1H-pyrrole-2-carboxamide |
| A104 | 535 | (R)-N-(4-(1-(4-(2-(dimethylamino)ethoxy)phenyl)pyrrolidin-2-yl)thiazol-2-yl)-1-((2- |
| | | fluoropyridin-4-yl)methyl)-1H-pyrrole-2-carboxamide |

### Route 13:

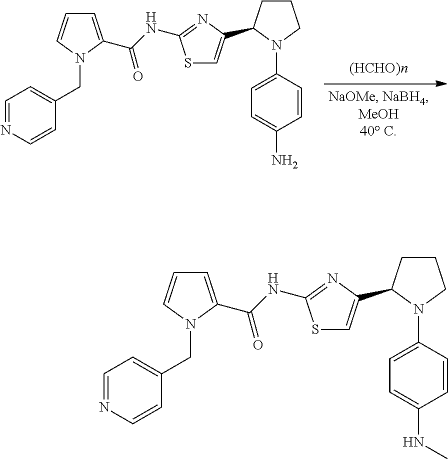 [SMILES: Nc1ccc(N2CCC[C@@H]2c2csc(NC(=O)c3cccn3Cc3ccncc3)n2)cc1 ; "(HCHO)n" ; "NaOMe, NaBH4,MeOH40° C." ; CNc1ccc(N2CCC[C@@H]2c2csc(NC(=O)c3cccn3Cc3ccncc3)n2)cc1]

A 100 mL vial with stir bar was charged with N-[4-[(2R)-1-(4-aminophenyl)pyrrolidin-2-yl]-1,3-thiazol-2-yl]-1-(pyridin-4-ylmethyl)pyrrole-2-carboxamide (100.00 mg, 0.23 mmol, 1.00 equiv), NaOMe (17.01 mg, 0.32 mmol, 1.40 equiv), Paraformaldehyde (28.37 mg, 0.32 mmol, 1.40 equiv) and MeOH (8 mL) under nitrogen atmosphere. The vial was capped and placed in a 40° C. bath. The reaction mixture was stirred at 40° C. overnight. The next morning, NaBH₄ (8.51 mg, 0.23 mmol, 1.00 equiv) was added. The reaction mixture was stirred at 40° C. for further 3 h. The reaction was then quenched by NaHCO₃ (aq). The resulting solution was extracted with ethyl acetate (3×30 mL) and washed with brine (1×20 mL). The combined organic layers were dried over Na₂SO₄, filtered and concentrated in vacuo. The resulting crude material was purified via prep-HPLC column to yield the desired product.

The following compounds were prepared via a similar method:

| | Observed | |
|---|---|---|
| | molecular | |
| | ion | Compound name |
| A15 | 459 | (R)-N-(4-(1-(4-(methylamino)phenyl)pyrrolidin-2-yl)thiazol-2-yl)-1-(pyridin- |
| | | 4-ylmethyl)-1H-pyrrole-2-carboxamide |

### Route 14:

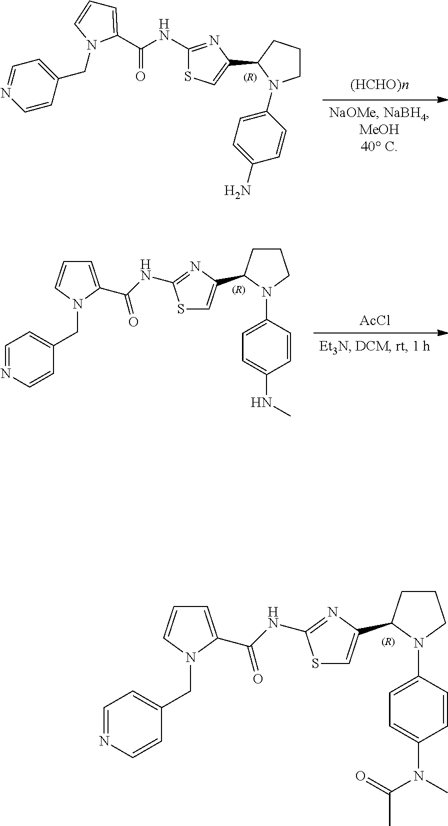

The reductive amination was performed as described in route 13.

The acylation was performed as described in route 3.

The following compounds were prepared via a similar method:

| | Observed | |
|---|---|---|
| | molecular | |
| | ion | Compound name |
| A47 | 501 | (R)-N-(4-(1-(4-(N-methylacetamido)phenyl)pyrrolidin-2-yl)thiazol-2-yl)-1-(pyridin- |
| | | 4-ylmethyl)-1H-pyrrole-2-carboxamide |

### Route 15:

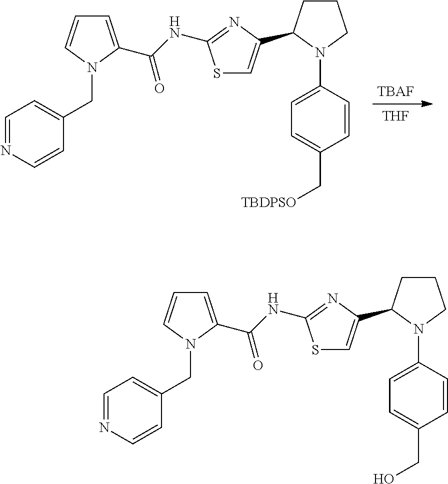

A 25 mL vial with stir bar was charged with silyl ether (50.00 mg, 0.07 mmol, 1.00 equiv.) and THF (4 mL, 0.02 M). TBAF (1M in THF, 0.22 mL, 0.22 mmol, 3.00 equiv.) was added. The flask was evacuated and flushed with nitrogen. The vial was capped and placed in a 25° C. bath. The reaction mixture was stirred at 25° C. overnight. The next morning, the reaction mixture was quenched by the addition of H₂O (15 mL). The mixture was extracted with DCM (3×20 mL), and the combined organic layers were washed with brine (2×20 mL). The organic layer was then dried over Na₂SO₄, filtered and concentrated in vacuo. The resulting crude material was purified via RP chromatography to yield the desired product.

The following compounds were prepared via a similar method:

| | Observed | |
|---|---|---|
| | molecular | |
| | ion | Compound name |
| A57 | 460 | (R)-N-(4-(1-(4-(hydroxymethyl)phenyl)pyrrolidin-2-yl)thiazol-2-yl)-1-(pyridin- |
| | | 4-ylmethyl)-1H-pyrrole-2-carboxamide |
| A78 | 492 | (R)-1-((2-fluoropyridin-4-yl)methyl)-N-(4-(1-(4-(2- |
| | | hydroxyethyl)phenyl)pyrrolidin-2-yl)thiazol-2-yl)-1H-pyrrole-2-carboxamide |
| A111 | 492 | 1-((2-fluoropyridin-4-yl)methyl)-N-(4-((R)-1-(4-((R)-1- |
| | | hydroxyethyl)phenyl)pyrrolidin-2-yl)thiazol-2-yl)-1H-pyrrole-2-carboxamide |
| A112 | 492 | 1-((2-fluoropyridin-4-yl)methyl)-N-(4-((R)-1-(4-((S)-1- |
| | | hydroxyethyl)phenyl)pyrrolidin-2-yl)thiazol-2-yl)-1H-pyrrole-2-carboxamide |
| A113 | 478 | (R)-1-((2-fluoropyridin-4-yl)methyl)-N-(4-(1-(4- |
| | | (hydroxymethyl)phenyl)pyrrolidin-2-yl)thiazol-2-yl)-1H-pyrrole-2- |
| | | carboxamide |
| A114 | 518 | (R)-1-((2-fluoropyridin-4-yl)methyl)-N-(4-(1-(4-(1- |
| | | hydroxycyclobutyl)phenyl)pyrrolidin-2-yl)thiazol-2-yl)-1H-pyrrole-2- |
| | | carboxamide |
| A134 | 508 | (R)-1-((2-fluoropyridin-4-yl)methyl)-N-(4-(1-(4-(2- |
| | | hydroxyethoxy)phenyl)pyrrolidin-2-yl)thiazol-2-yl)-1H-pyrrole-2-carboxamide |

### Route 16:

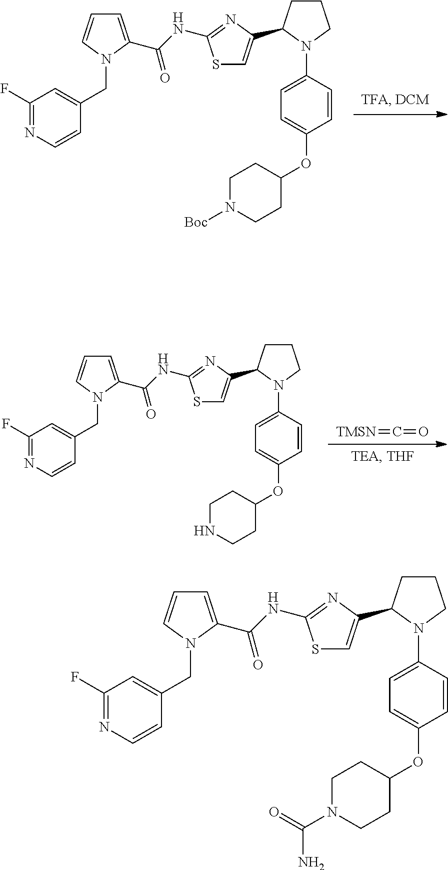

The Boc deprotection was performed as described in route 1.

A 100 mL vial with stir bar was charged with 1-[(2-fluoropyridin-4-yl)methyl]-N-{4-[(2R)-1-[4-(piperidin-4-yloxy)phenyl]pyrrolidin-2-yl]-1,3-thiazol-2-yl}pyrrole-2-carboxamide (150 mg, 0.27 mmol, 1.00 equiv.), TEA (0.114 mL, 0.82 mmol, 3.00 equiv.) and THF (8 mL, 0.03 M). Isocyanatotrimethylsilane (45 μL, 0.33 mmol, 1.20 equiv.) was added. The flask was evacuated and flushed with nitrogen. The vial was capped and placed in a 60° C. bath. The reaction mixture was stirred at 60° C. for 1 h. The reaction mixture was cooled to room temperature. The reaction mixture was quenched by the addition of H₂O (15 mL). The mixture was extracted with EtOAc (3×15 mL). The organic layer was then dried over Na₂SO₄, filtered and concentrated in vacuo. The resulting crude material was purified via RP chromatography to yield the desired product.

The following compounds were prepared via a similar method:

| | Observed | |
|---|---|---|
| | molecular | |
| | ion | Compound name |
| A135 | 590 | (R)-4-(4-(2-(2-(1-((2-fluoropyridin-4-yl)methyl)-1H-pyrrole-2-carboxamido)thiazol- |
| | | 4-yl)pyrrolidin-1-yl)phenoxy)piperidine-1-carboxamide |
| A123 | 506 | (R)-1-((2-fluoropyridin-4-yl)methyl)-N-(4-(1-(4-ureidophenyl)pyrrolidin-2- |
| | | yl)thiazol-2-yl)-1H-pyrrole-2-carboxamide |
| A130 | 562 | (R)-N-(4-(1-(4-((1-carbamoylazetidin-3-yl)oxy)phenyl)pyrrolidin-2-yl)thiazol-2-yl)- |
| | | 1-((2-fluoropyridin-4-yl)methyl)-1H-pyrrole-2-carboxamide |
| A156 | 591 | (R)-4-((6-(2-(2-(1-((2-fluoropyridin-4-yl)methyl)-1H-pyrrole-2- |
| | | carboxamido)thiazol-4-yl)pyrrolidin-1-yl)pyridin-3-yl)oxy)piperidine-1- |
| | | carboxamide |
| A159 | 591 | (R)-4-((5-(2-(2-(1-((2-fluoropyridin-4-yl)methyl)-1H-pyrrole-2- |
| | | carboxamido)thiazol-4-yl)pyrrolidin-1-yl)pyridin-2-yl)oxy)piperidine-1- |
| | | carboxamide |
| A166 | 604 | (R)-4-(4-(2-(2-(1-((2-fluoropyridin-4-yl)methyl)-4-methyl-1H-pyrrole-2- |
| | | carboxamido)thiazol-4-yl)pyrrolidin-1-yl)phenoxy)piperidine-1-carboxamide |

### Route 17:

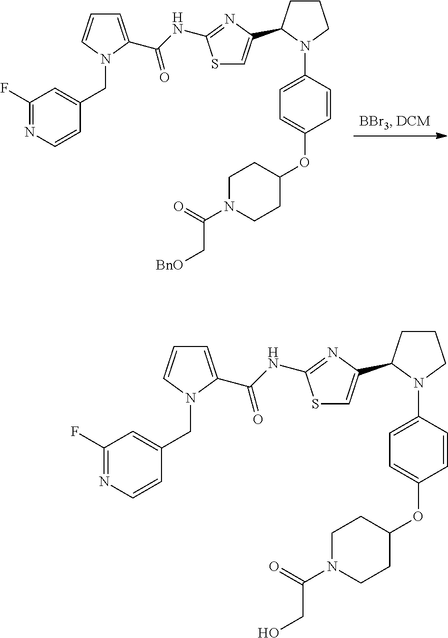

A 25 mL vial with stir bar was charged with N-{4-[(2R)—N-[4-({1-[2-(benzyloxy)acetyl]piperidin-4-yl}oxy)phenyl]pyrrolidin-2-yl]-1,3-thiazol-2-yl}-1-[(2-fluoropyridin-4-yl)methyl]pyrrole-2-carboxamide (100 mg, 0.14 mmol, 1.00 equiv.) and DCM (7 mL, 0.02 M). The flask was evacuated and flushed with nitrogen. BBr3 (1 M in DCM, 0.43 mL, 0.43 mmol, 3.00 equiv.) was added at 0° C. The vial was capped and placed in an 25° C. bath. The reaction mixture was stirred at 25° C. for 1 h. The reaction mixture was quenched by the addition of NaHCO₃ (s). The resulting mixture was diluted with MeOH (10 mL). The resulting mixture was filtered, the filter cake was washed with MeOH (10 mL). The combined filtrate was concentrated in vacuo. The resulting crude material was purified via RP chromatography to yield the desired product.

The following compounds were prepared via a similar method:

| | Observed | |
|---|---|---|
| | molecular | |
| | ion | Compound name |
| A139 | 605 | (R)-1-((2-fluoropyridin-4-yl)methyl)-N-(4-(1-(4-((1-(2-hydroxyacetyl)piperidin-4- |
| | | yl)oxy)phenyl)pyrrolidin-2-yl)thiazol-2-yl)-1H-pyrrole-2-carboxamide |
| A141 | 521 | (R)-1-((2-fluoropyridin-4-yl)methyl)-N-(4-(1-(4-(2- |
| | | hydroxyacetamido)phenyl)pyrrolidin-2-yl)thiazol-2-yl)-1H-pyrrole-2-carboxamide |
| C114 | 421 | (E)-N-(4-(2-(1-(2-hydroxyethyl)-1H-imidazol-4-yl)vinyl)thiazol-2-yl)-1-(pyridin-4- |
| | | ylmethyl)-1H-pyrrole-2-carboxamide |
| A110 | 577 | (R)-1-((2-fluoropyridin-4-yl)methyl)-N-(4-(1-(4-((1-(2-hydroxyacetyl)azetidin-3- |
| | | yl)oxy)phenyl)pyrrolidin-2-yl)thiazol-2-yl)-1H-pyrrole-2-carboxamide |

### Route 18:

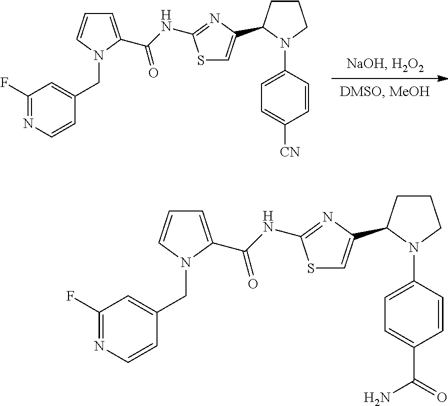

A 100 mL vial with stir bar was charged with N-[4-[(2R)-1-(4-cyanophenyl)pyrrolidin-2-yl]-1,3-thiazol-2-yl]-1-[(2-fluoropyridin-4-yl)methyl]pyrrole-2-carboxamide (200.00 mg, 0.42 mmol, 1.00 equiv.), DMSO (4 mL) and MeOH (8 mL, 0.04 M). NaOH (33.86 mg, 0.85 mmol, 2.00 equiv.) and H₂O₂ (30 wt % in water, 238.00 mg, 2.10 mmol, 5.00 equiv.) were added. The vial was capped and placed in a 50° C. bath. The reaction mixture was stirred at 50° C. for 2 h. The reaction mixture was cooled to room temperature. The reaction mixture was quenched by the addition of H₂O (40 mL). The mixture was extracted with EtOAc (3×50 mL), and the combined organic layers were washed with brine (2×50 mL). The organic layer was then dried over Na₂SO₄, filtered and concentrated in vacuo. The resulting crude material was purified via prep-HPLC chromatography to yield the desired product.

The following compounds were prepared via a similar method:

| | Observed | |
|---|---|---|
| | molecular | |
| | ion | Compound name |
| A65 | 491 | (R)-N-(4-(1-(4-carbamoylphenyl)pyrrolidin-2-yl)thiazol-2-yl)-1-((2-fluoropyridin-4- |
| | | yl)methyl)-1H-pyrrole-2-carboxamide |
| C105 | 480 | (R)-N-(4-(1-(4-carbamoylphenyl)pyrrolidin-2-yl)thiazol-2-yl)-1-((tetrahydro-2H- |
| | | pyran-4-yl)methyl)-1H-pyrrole-2-carboxamide |
| A152 | 491 | (R)-1-((2-fluoropyridin-4-yl)methyl)-N2-(4-(1-phenylpyrrolidin-2-yl)thiazol-2-yl)- |
| | | 1H-pyrrole-2,4-dicarboxamide |
| B109 | 466 | (E)-N-(4-(2-(1-(3-amino-3-oxopropyl)-1H-imidazol-4-yl)vinyl)thiazol-2-yl)-1-((2- |
| | | fluoropyridin-4-yl)methyl)-1H-pyrrole-2-carboxamide |
| B111 | 480 | (E)-N-(4-(2-(1-(4-amino-4-oxobutan-2-yl)-1H-imidazol-4-yl)vinyl)thiazol-2-yl)-1- |
| | | ((2-fluoropyridin-4-yl)methyl)-1H-pyrrole-2-carboxamide |
| B113 | 466 | (E)-N-(4-(2-(1-(1-amino-1-oxopropan-2-yl)-1H-imidazol-4-yl)vinyl)thiazol-2-yl)-1- |
| | | ((2-fluoropyridin-4-yl)methyl)-1H-pyrrole-2-carboxamide |

### Route 19:

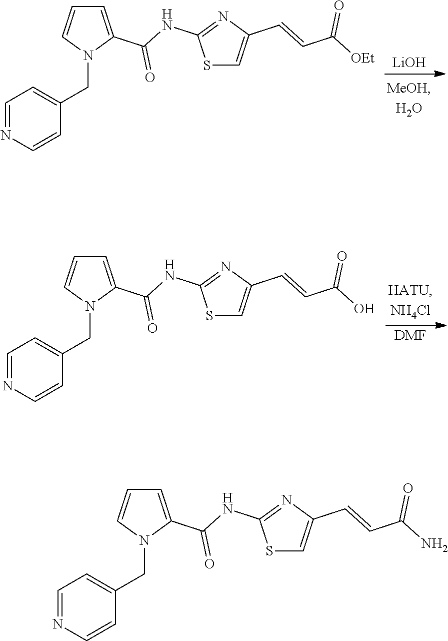

A 50 mL vial with stir bar was charged with ethyl (2E)-3-{2-[1-(pyridin-4-ylmethyl)pyrrole-2-amido]-1,3-thiazol-4-yl}prop-2-enoate (1.50 g, 3.92 mmol, 1.00 equiv.), MeOH (12.00 mL, 0.25 M) and H₂O (4.00 mL). LiOH (476 mg, 19.88 mmol, 5.07 equiv.) was added. The vial was capped and placed in a 40° C. bath. The reaction mixture was stirred at 40° C. for 4 h. The reaction mixture was cooled to room temperature. The pH of the solution was adjusted to 7 with 1 M HCl (aq.). The precipitated solids were collected by filtration and washed with H₂O (2×8 mL). The filter cake was dried under vacuum. The crude product was used in the next step without further purification.

A 50 mL vial with stir bar was charged with (2E)-3-{2-[1-(pyridin-4-ylmethyl)pyrrole-2-amido]-1,3-thiazol-4-yl}prop-2-enoic acid (100 mg, 0.28 mmol, 1.00 equiv.), DIEA (0.15 mL, 0.85 mmol, 3.00 equiv.), HATU (160.94 mg, 0.42 mmol, 1.50 equiv.) and DMF (8 mL, 0.04 M). The vial was capped and placed in a 25° C. bath. The reaction mixture was stirred at 25° C. for 10 min. NH₄Cl (22.64 mg, 0.42 mmol, 1.50 equiv.) was added. The flask was then evacuated and flushed with nitrogen atmosphere. The reaction mixture was stirred at 25° C. for 2 h. The reaction mixture was quenched by the addition of H₂O (50 mL). The mixture was extracted with EtOAc (3×50 mL), and the combined organic layers were washed with brine (3×50 mL). The organic layer was then dried over Na₂SO₄, filtered and concentrated in vacuo. The resulting crude material was purified via RP chromatography to yield the desired product.

### Route 20:

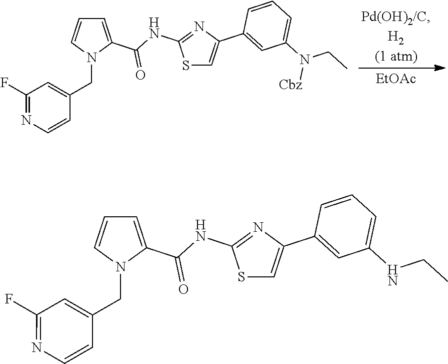 [SMILES: "EtOAc" ; CCN(C(=O)OCc1ccccc1)c1cccc(-c2csc(NC(=O)c3cccn3Cc3ccnc(F)c3)n2)c1 ; "Pd(OH)2/C,H2(1 atm)" ; CCNc1cccc(-c2csc(NC(=O)c3cccn3Cc3ccnc(F)c3)n2)c1]

A 50 mL vial with stir bar was charged with benzyl ethyl(3-(2-(1-((2-fluoropyridin-4-yl)methyl)-1H-pyrrole-2-carboxamido)thiazol-4-yl)phenyl)carbamate (150.00 mg, 0.36 mmol, 1.00 equiv.), Pd(OH)₂/C (20 wt %, 150 mg, 1.07 mmol, 2.97 equiv.) and EtOAc (10 mL, 0.04 M) under nitrogen atmosphere. The flask was then evacuated and flushed with hydrogen. The reaction mixture was hydrogenated at room temperature for 45 min under hydrogen atmosphere using a hydrogen balloon. Then the reaction mixture was filtered through a celite pad and the filtrate was concentrated under reduced pressure. The resulting crude material was purified via prep-HPLC chromatography to yield the desired product.

### Route 21:

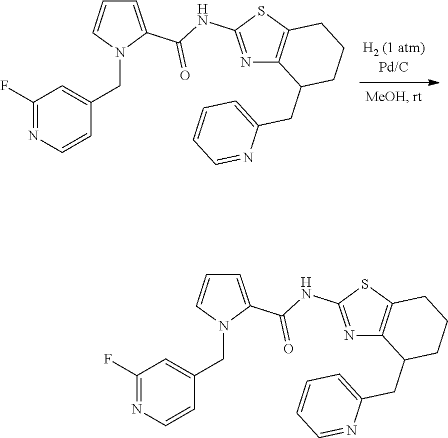 [SMILES: O=C(Nc1nc2c(s1)CCCC2Cc1ccccn1)c1cccn1Cc1ccnc(F)c1 ; O=C(Nc1nc2c(s1)CCCC2Cc1ccccn1)c1cccn1Cc1ccnc(F)c1]

A 50 mL vial with stir bar was charged with (Z)-1-((2-fluoropyridin-4-yl)methyl)-N-(4-(pyridin-2-ylmethylene)-4,5,6,7-tetrahydrobenzo[d]thiazol-2-yl)-1H-pyrrole-2-carboxamide (100.00 mg, 0.22 mmol, 1.00 equiv.), Pd/C (10 wt %, 100.09 mg, 0.94 mmol, 4.20 equiv.) and MeOH (10 mL, 0.02 M) under nitrogen atmosphere. The flask was then evacuated and flushed with hydrogen. The reaction mixture was hydrogenated at room temperature for 2 h under hydrogen atmosphere using a hydrogen balloon. Then the reaction mixture was filtered through a celite pad and the filtrate was concentrated under reduced pressure. The resulting crude material was purified via prep-HPLC chromatography to yield the desired product.

The following compounds were prepared via a similar method:

| | Observed | |
|---|---|---|
| | molecular ion | Compound name |
| A176 | 573 | (R)-N-(4-(1-(4-(2-(1-acetylazetidin-3-yl)ethyl)phenyl)pyrrolidin-2-yl)thiazol-2- |
| | | yl)-1-((2-fluoropyridin-4-yl)methyl)-1H-pyrrole-2-carboxamide |

### Route 22:

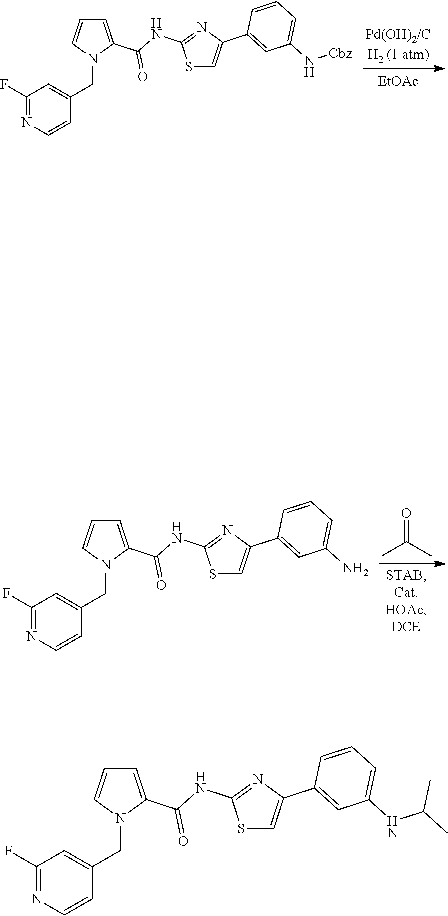

The CBz deprotection was performed as described in route 20.

A 50 mL vial with stir bar was charged with N-[4-(3-aminophenyl)-1,3-thiazol-2-yl]-1-[(2-fluoropyridin-4-yl)methyl]pyrrole-2-carboxamide (70.00 mg, 0.18 mmol, 1.00 equiv.), acetone (20.67 mg, 0.36 mmol, 2.00 equiv.), HOAc (2 mL, 0.04 mmol, 0.20 equiv.) and DCE (6 mL, 0.03 M). STAB (56.56 mg, 0.27 mmol, 1.50 equiv.) was added. And the vial was capped and placed in an 25° C. bath. The reaction mixture was stirred at 25° C. for 12 h. The reaction mixture was quenched by the addition of H₂O (15 mL). The mixture was extracted with DCM (3×20 mL), and the combined organic layers were washed with brine (2×20 mL). The organic layer was then dried over Na₂SO₄, filtered and concentrated in vacuo. The resulting crude material was purified via RP chromatography to yield the desired product.

### Route 23:

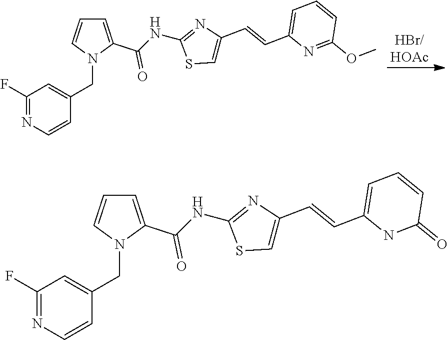

A 100 mL vial with stir bar was charged with 1-[(2-fluoropyridin-4-yl)methyl]-N-{4-[(E)-2-(6-methoxypyridin-2-yl)ethenyl]-1,3-thiazol-2-yl}pyrrole-2-carboxamide (100 mg, 0.23 mmol, 1 equiv.) and HBr (40 wt % in AcOH, 10 mL, 0.02 M). And the vial was capped and placed in an 90° C. bath. The reaction mixture was stirred at 90° C. for 2 h. The reaction mixture was cooled to room temperature. The reaction mixture was concentrated in vacuo. The pH of the solution was adjusted to 7 with sat. NaHCO₃ (aq.). The mixture was extracted with DCM (3×40 mL), and the combined organic layers were washed with brine (1×30 mL). The organic layer was then dried over Na₂SO₄, filtered and concentrated in vacuo. The resulting crude material was purified via RP chromatography to yield the desired product.

The following compounds were prepared via a similar method:

| | Observed | |
|---|---|---|
| | molecular ion | Compound name |
| B61 | 422 | (E)-1-((2-fluoropyridin-4-yl)methyl)-N-(4-(2-(6-oxo-1,6- |
| | | dihydropyridin-2-yl)vinyl)thiazol-2-yl)-1H-pyrrole-2-carboxamide |

### Route 24:

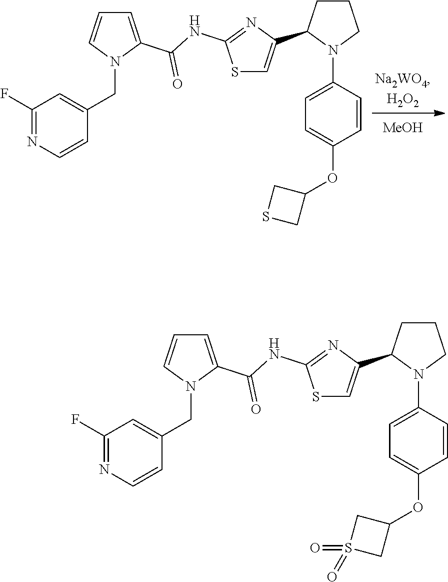

A 25 mL vial with stir bar was charged with 1-[(2-fluoropyridin-4-yl)methyl]-N-{4-[(2R)-1-[4-(thietan-3-yloxy)phenyl]pyrrolidin-2-yl]-1,3-thiazol-2-yl}pyrrole-2-carboxamide (50.0 mg, 0.09 mmol, 1.00 equiv.) and MeOH (5 mL, 0.02 M). Na₂WO₄ (13.5 mg, 0.05 mmol, 0.49 equiv.) and H₂O₂ (30 wt % in water, 149.5 mg, 1.32 mmol, 14.67 equiv.) were added. The vial was capped and placed in a 25° C. bath. The reaction mixture was stirred at 25° C. for 2 h. The resulting mixture was filtered, the filter cake was washed with MeOH (2×10 mL). The combined filtrate was concentrated in vacuo. The resulting crude material was purified via RP chromatography to yield the desired product.

The following compounds were prepared via a similar method:

| | Observed | |
|---|---|---|
| | molecular ion | Compound name |
| A158 | 568 | (R)-N-(4-(1-(4-((1,1-dioxidothietan-3-yl)oxy)phenyl)pyrrolidin-2- |
| | | yl)thiazol-2-yl)-1-((2-fluoropyridin-4-yl)methyl)-1H-pyrrole-2-carboxamide |

### Route 25:

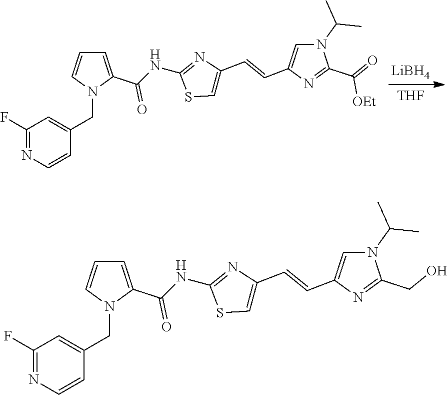 [SMILES: CCOC(=O)c1nc(/C=C/c2csc(NC(=O)c3cccn3Cc3ccnc(F)c3)n2)cn1C(C)C ; "LiBH4" ; "THF" ; CC(C)n1cc(/C=C/c2csc(NC(=O)c3cccn3Cc3ccnc(F)c3)n2)nc1CO]

A 50 mL vial with stir bar was charged with ethyl (E)-4-(2-(2-(1-((2-fluoropyridin-4-yl)methyl)-1H-pyrrole-2-carboxamido)thiazol-4-yl)vinyl)-1-isopropyl-1H-imidazole-2-carboxylate (180 mg, 0.35 mmol, 1.00 equiv.) and THF (8.00 mL, 0.04 M). LiBH₄ (30.84 mg, 1.42 mmol, 4.00 equiv.) was added at 0° C., and the vial was capped and placed in an 25° C. bath. The reaction mixture was stirred at 25° C. for 1 h. The reaction was then quenched by the addition of water (20 mL). The resulting solution was extracted with DCM (3×30 mL). The organic layer was dried over Na₂SO₄, filtered and concentrated in vacuo. The resulting crude material was purified via RP chromatography to yield the desired product.

The following compounds were prepared via a similar method:

| | Observed | |
|---|---|---|
| | molecular ion | Compound name |
| B103 | 467 | (E)-1-((2-fluoropyridin-4-yl)methyl)-N-(4-(2-(2-(hydroxymethyl)-1-isopropyl- |
| | | 1H-imidazol-4-yl)vinyl)thiazol-2-yl)-1H-pyrrole-2-carboxamide |
| B118 | 467 | (E)-1-((2-fluoropyridin-4-yl)methyl)-N-(4-(2-(1-(1-hydroxypropan-2-yl)-5- |
| | | methyl-1H-imidazol-4-yl)vinyl)thiazol-2-yl)-1H-pyrrole-2-carboxamide |

### Route 26:

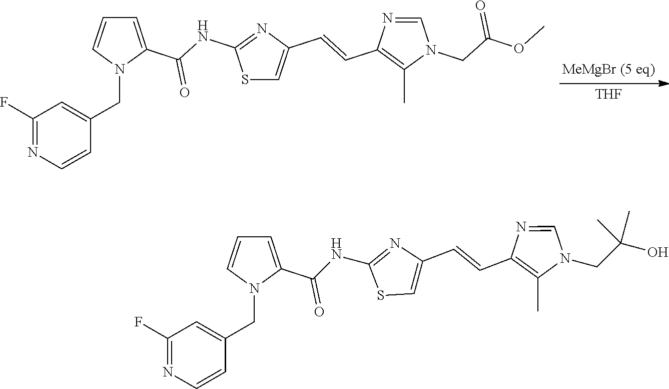 [SMILES: COC(=O)Cn1cnc(/C=C/c2csc(NC(=O)c3cccn3Cc3ccnc(F)c3)n2)c1C ; "MeMgBr (5 eq)" ; "THF" ; Cc1c(/C=C/c2csc(NC(=O)c3cccn3Cc3ccnc(F)c3)n2)ncn1CC(C)(C)O]

A 50 mL vial with stir bar was charged with methyl (E)-2-(4-(2-(2-(1-((2-fluoropyridin-4-yl)methyl)-1H-pyrrole-2-carboxamido)thiazol-4-yl)vinyl)-5-methyl-1H-imidazol-1-yl)acetate (60 mg, 0.13 mmol, 1.00 equiv.) and THF (5 mL, 0.03 M). MeMgBr (3 M in THF, 0.21 mL, 0.63 mmol, 5.00 equiv.) was added at 0° C. The flask was evacuated and flushed with nitrogen. The vial was capped and placed in an 25° C. bath. The reaction mixture was stirred at 25° C. overnight. The next morning, the reaction mixture was quenched by sat. NH₄Cl (aq.) (15 mL). The mixture was extracted with EtOAc (3×15 mL). The organic layer was then dried over Na₂SO₄, filtered and concentrated in vacuo. The resulting crude material was purified via RP chromatography to yield the desired product.

The following compounds were prepared via a similar method:

| | Observed | |
|---|---|---|
| | molecular | |
| | ion | Compound name |
| B121 | 481 | (E)-1-((2-fluoropyridin-4-yl)methyl)-N-(4-(2-(1-(2-hydroxy- |
| | | 2-methylpropyl)-5-methyl-1H-imidazol-4-yl)vinyl)thiazol-2-yl)- |
| | | 1H-pyrrole-2-carboxamide |

### Route 27:

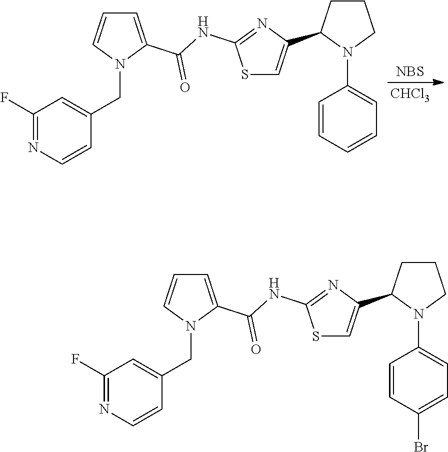

A vial with stir bar was charged with aniline (43 mg, 0.096 mmol, 1.0 equiv.) and NBS (19 mg, 0.11 mmol, 1.1 equiv.). Chloroform (1 mL, 0.1 M) was added, and the reaction mixture was allowed to stir at room temperature overnight. The next morning, the reaction was concentrated in vacuo, and the resulting crude material was purified via silica gel chromatography to yield the desired product.

The following compounds were made via a similar method:

| | Observed | |
|---|---|---|
| | molecular ion | Compound name |
| A116 | 526 | (R)-N-(4-(1-(4-bromophenyl)pyrrolidin-2-yl)thiazol- |
| | | 2-yl)-1-((2-fluoropyridin-4-yl)methyl)-1H-pyrrole- |
| | | 2-carboxamide |

Syntheses of Amide Coupling Intermediates

Aryl Bromide Syntheses

### Route 1:

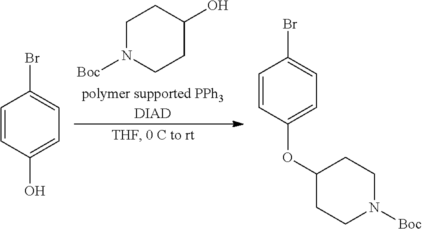

A flame-dried 100 mL roundbottom flask with stir bar was charged with polymer-supported PPh₃ (3.31 g, 9.94 mmol, 2 equiv.), 4-bromophenol (964 mg, 5.47 mmol, 1.1 equiv.) and tert-butyl 4-hydroxypiperidine-1-carboxylate (1.00 g, 4.97 mmol, 1 equiv.). The reaction mixture was evacuated and backflushed with nitrogen. Dry THF (20 mL, 0.23 M) was added, and the reaction mixture was cooled to 0° C. DIAD (1.95 mL, 9.94 mmol, 2 equiv.) was slowly added at 0° C., and the reaction mixture was allowed to warm to room temperature overnight. The next morning, the reaction mixture was filtered and washed with EtOAc (2×50 mL). The resulting crude material was concentrated in vacuo and purified via silica gel chromatography to yield the desired product.

The following compounds were prepared via a similar method:

| | Compound Name |
|---|---|
| A100 | tert-butyl 4-(4-bromo-3-fluorophenoxy)piperidine- |
| | 1-carboxylate |
| A101, | tert-butyl (2-(4-bromophenoxy)ethyl)carbamate |
| A104, | |
| and A121 | |
| A102 | tert-butyl 4-(4-bromo-2-fluorophenoxy)piperidine- |
| | 1-carboxylate |
| A103 | tert-butyl (2-(4-bromophenoxy)ethyl)(methyl)carbamate |
| and 117 | |
| A105 | tert-butyl 4-(4-bromo-3,5-difluorophenoxy)piperidine- |
| | 1-carboxylate |
| A106 | tert-butyl 4-(4-bromo-2,6-difluorophenoxy)piperidine- |
| | 1-carboxylate |
| A126 | tert-butyl 4-(4-bromo-2-cyanophenoxy)piperidine- |
| | 1-carboxylate |
| A129 | tert-butyl 4-(4-amino-2-methoxyphenoxy)piperidine- |
| | 1-carboxylate |
| A133 | tert-butyl 4-(4-bromo-3-cyanophenoxy)piperidine- |
| | 1-carboxylate |
| A140 | tert-butyl 4-(4-bromo-2-chlorophenoxy)piperidine- |
| | 1-carboxylate |
| A143 | tert-butyl 4-(4-bromo-3-chlorophenoxy)piperidine- |
| | 1-carboxylate |
| A154 | tert-butyl 4-((6-bromopyridin-3-yl)oxy)piperidine- |
| and A156 | 1-carboxylate |
| A155 | tert-butyl 4-((5-bromopyridin-2-yl)oxy)piperidine- |
| and A159 | 1-carboxylate |

### Route 2:

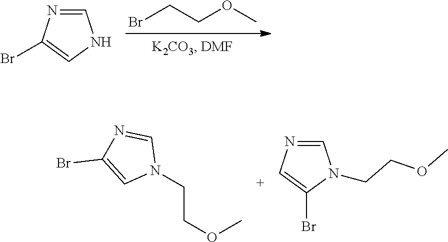

A 20 mL vial with stir bar was charged with 4-bromo-1H-imidazole (500 mg, 3.40 mmol, 1.00 equiv.), 2-bromoethyl methyl ether (567.41 mg, 4.08 mmol, 1.20 equiv.) and K₂CO₃ (1.41 g, 10.21 mmol, 3.00 equiv.). DMF (10 mL, 0.34 M) was added under nitrogen atmosphere, and the vial was capped and placed in an 90° C. bath. The reaction mixture was stirred at 90° C. for 3 h. The reaction mixture was cooled to room temperature. The reaction was then quenched by water (50 mL). The resulting solution was extracted with EtOAc (3×50 mL), and the combined organic layers were washed with brine (3×100 mL). The organic layer was then dried over Na₂SO₄, filtered and concentrated in vacuo. The resulting crude material was purified via silica gel chromatography to yield the desired product. The desired isomer was confirmed by NOESY spectroscopy.

The following compounds were prepared via a similar method:

| | Compound name | Leaving Group/Base used |
|---|---|---|
| B91 | 1-(4-(4-bromo-1H-imidazol-1-yl)piperidin- | OMs used |
| | 1-yl)ethan-1-one | K₂CO₃ base |
| B105 | 1-benzyl-4-bromo-1H-imidazole | Br used |
| | | K₂CO₃ base |
| B58 and | 4-bromo-1-isopropyl-5-methyl-1H-imidazole | I used |
| B116 | | Cs₂CO₃ base |
| B117 | 4-bromo-1-ethyl-5-methyl-1H-imidazole | I used |
| | | Cs₂CO₃ base |
| B118 and | ethyl 2-(4-bromo-5-methyl-1H-imidazol-1- | Br used |
| B119 | yl)propanoate | Cs₂CO₃ base |
| B121 and | methyl 2-(4-bromo-5-methyl-1H-imidazol-1- | Br used |
| B120 | yl)acetate | K₂CO₃ base |
| B129 | 4-bromo-5-chloro-1-isopropyl-1H-imidazole | I used |
| | | K₂CO₃ base |

### Route 3:

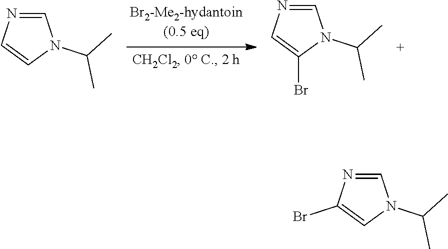

A 100 mL vial with stir bar was charged with 1-isopropylimidazole (2.00 g, 18.16 mmol, 1.00 equiv.) in DCM (100 mL). 1,3-dibromo-5,5-dimethylhydantoin (2.60 g, 9.08 mmol, 0.5 equiv.) in DCM (100 mL, 0.09 M) was added dropwise at 0° C. under nitrogen atmosphere, and the vial was capped and placed in an 25° C. bath. The reaction mixture was stirred at 25° C. for 4 h. The reaction mixture was poured into sat. Na₂SO₃ (aq.) (100 mL). The resulting solution was extracted with EtOAc (2×150 mL) and the combined organic layers were washed with brine (2×70 mL). and washed with H₂O (1×100 mL), followed by brine (2×200 mL). The organic layer was then dried over Na₂SO₄, filtered and concentrated in vacuo. The resulting crude material was purified via silica gel chromatography to yield the desired products as separate isomers. The desired isomer was confirmed by NOESY spectroscopy.

The following compounds were prepared via a similar method:

| | Compound name |
|---|---|
| B134 | 4-bromo-1-(tert-butyl)-1H-imidazole |

### Route 4:

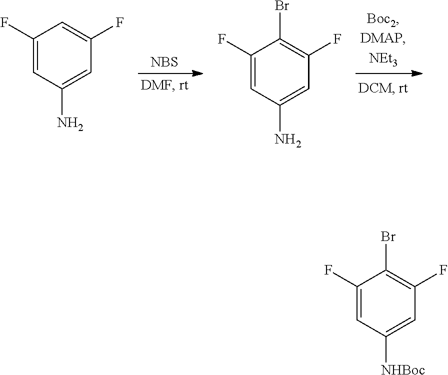

A 100 mL roundbottom flask with stir bar was charged with 3,5-difluoroaniline (1.00 g, 7.75 mmol, 1.0 equiv.) and N-bromosuccinimide (1.52 g, 8.52 mmol, 1.1 equiv.). DMF (15 mL, 0.5 M) was added, and the reaction mixture was allowed to stir at room temperature overnight. The next morning, the reaction mixture was diluted with EtOAc (150 mL) and washed with saturated NaHCO₃ (2×150 mL). The combined aqueous layers were extracted with EtOAc (2×150 mL), and the combined organic layers were dried over Na₂SO₄, filtered and concentrated in vacuo. The resulting crude material was purified via silica gel chromatography and taken on to the next step.

A 100 mL roundbottom flask with stir bar was charged with 4-bromo-3,5-difluoroaniline (1.28 g, 6.15 mmol, 1.0 equiv.), triethylamine (0.94 mL, 6.77 mmol, 1.1 equiv.) and DMAP (75 mg, 0.615 mmol, 0.1 equiv.). DCM (15 mL, 0.35 M) was added, followed by Boc₂O (1.6 mL, 6.77 mmol, 1.1 equiv.). The reaction mixture was allowed to stir at room temperature overnight. The next morning, the reaction mixture was diluted with DCM (100 mL) and washed with saturated NH₄Cl (2×100 mL). The combined aqueous layers were extracted with DCM (1×100 mL), and the combined organic layers were dried over Na₂SO₄, filtered and concentrated in vacuo. The resulting crude material was purified via silica gel chromatography to yield the desired product.

### Route 5:

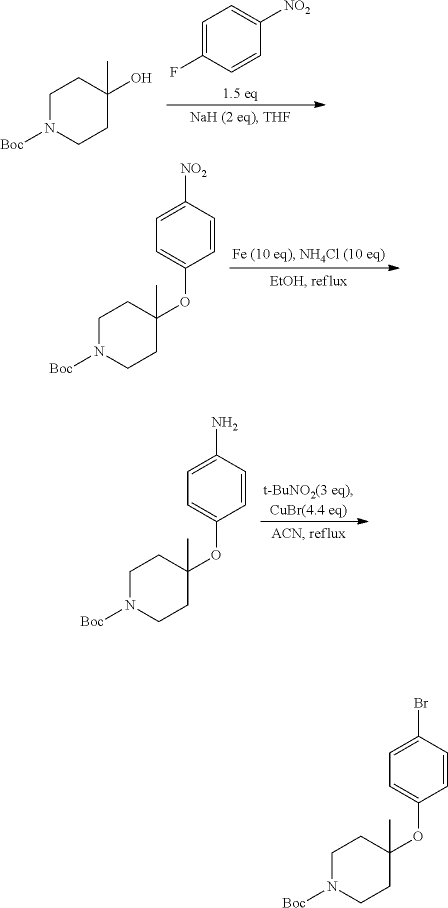

A 250 mL round bottom flask with stir bar was charged with tert-butyl 4-hydroxy-4-methylpiperidine-1-carboxylate (5.00 g, 23.22 mmol, 1.00 equiv.) and THF (80 mL, 0.29 M). NaH (60 wt % in mineral oil, 1.86 g, 46.50 mmol, 2.00 equiv.) was slowly added, and the reaction mixture was allowed to stir at 0° C. for 20 min. 4-fluoronitrobenzene (4.92 g, 34.84 mmol, 1.50 equiv.) was added, and the reaction mixture was allowed to stir at 60° C. overnight. The next morning, the reaction mixture was cooled to room temperature. The reaction mixture was quenched by the addition of H₂O (150 mL). The mixture was extracted with EtOAc (3×150 mL) and the combined organic layers were washed with brine (2×150 mL). The combined organic layers were dried over Na₂SO₄, filtered and concentrated in vacuo. The resulting crude material was purified via silica gel chromatography to yield the desired product.

A 250 mL vial with stir bar was charged with tert-butyl 4-methyl-4-(4-nitrophenoxy)piperidine-1-carboxylate (6.00 g, 17.84 mmol, 1.00 equiv.), Fe (10 g, 179.06 mmol, 10.00 equiv.), NH₄Cl (9.40 g, 175.73 mmol, 10.00 equiv.) and EtOH (150 mL, 0.12 M) under nitrogen atmosphere, and the vial was capped and placed in an 70° C. bath. The reaction mixture was stirred at 70° C. overnight. The reaction mixture was cooled to room temperature. The reaction mixture was concentrated in vacuo. The resulting material was charged with H₂O (80 mL). The mixture was extracted with EtOAc (3×100 mL) and washed with brine (1×150 mL). The combined organic layers were dried over Na₂SO₄, filtered and concentrated in vacuo. The resulting crude material was purified via silica gel chromatography to yield the desired product.

A 100 mL vial with stir bar was charged with tert-butyl 4-(4-aminophenoxy)-4-methylpiperidine-1-carboxylate (2.00 g, 6.53 mmol, 1.00 equiv.) and ACN (60 mL, 0.11 M). CuBr (4.00 g, 27.88 mmol, 4.40 equiv.) and tert-butyl nitrite (2.00 g, 19.40 mmol, 3.00 equiv.) were added under nitrogen atmosphere, and the vial was capped and placed in an 60° C. bath. The reaction mixture was stirred at 60° C. for 1 h. The reaction mixture was cooled to room temperature. The reaction mixture was poured into EtOAc (300 mL) and washed with H₂O (1×150 mL), followed by brine (2×150 mL). The organic layer was then dried over Na₂SO₄, filtered and concentrated in vacuo. The resulting crude material was purified via silica gel chromatography to yield the desired product.

### Route 6:

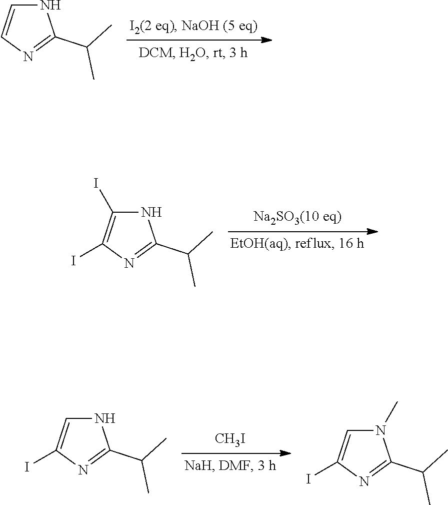

A 250 mL vial with stir bar was charged with 2-isopropyl-1H-imidazole (2.00 g, 18.16 mmol, 1.00 equiv.) in DCM (40 mL, 0.23 M) and H₂O (40 mL). NaOH (1.45 g, 36.31 mmol, 2.00 equiv.) and iodine (9.22 g, 36.31 mmol, 2.00 equiv.) were added, and the vial was capped and placed in an 25° C. bath. The reaction mixture was stirred at 25° C. for 2 h. The mixture was extracted with DCM (3×100 mL). The combined organic layers were dried over Na₂SO₄, filtered and concentrated in vacuo. The crude product was used in the next step without further purification.

A 250 mL vial with stir bar was charged with 4,5-diiodo-2-isopropyl-1H-imidazole (2 g, 5.53 mmol, 1.00 equiv.) and EtOH (60 mL, 0.09 M). Na2SO3 (6.96 g, 55.26 mmol, 10.00 equiv.) was added, and the vial was capped and placed in an 70° C. bath. The reaction mixture was stirred at 70° C. overnight. The next morning, the reaction mixture was cooled to room temperature. The reaction mixture was concentrated in vacuo. The resulting material was charged with H₂O (50 mL). The mixture was extracted with DCM (3×100 mL). The combined organic layers were dried over Na₂SO₄, filtered and concentrated in vacuo. The crude product was used in the next step without further purification.

A 250 mL round bottom flask with stir bar was charged with 4-iodo-2-isopropyl-1H-imidazole (1.00 g, 4.24 mmol, 1.00 equiv.) and DMF (10 mL, 0.42 M). NaH (60 wt % in mineral oil, 150 mg, 6.35 mmol, 1.50 equiv.) was slowly added, and the reaction mixture was allowed to stir at 0° C. for 20 min. CH₃I (0.32 mL, 5.08 mmol, 1.20 equiv.) was added at 0° C., and the vial was capped and placed in an 25° C. bath. The reaction mixture was allowed to stir at 25° C. for 2 h. The reaction mixture was quenched with H₂O (50 mL). The mixture was extracted with DCM (3×50 mL) and the combined organic layers were washed with brine (2×150 mL). The combined organic layers were dried over Na₂SO₄, filtered and concentrated in vacuo. The resulting crude material was purified via silica gel chromatography to yield the desired product.

### Route 7:

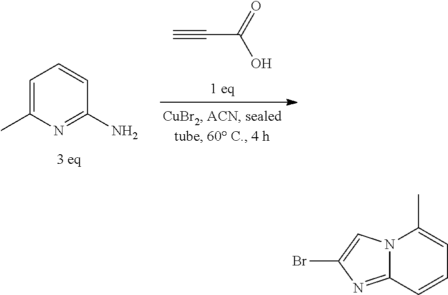

A 250 mL sealed tube with stir bar was charged with 6-methylpyridin-2-amine (4.32 g, 39.95 mmol, 3.00 equiv.), CuBr₂ (4.14 g, 19.74 mmol, 1.50 equiv.), propiolic acid (936 mg, 13.36 mmol, 1.00 equiv.), and ACN (30.00 mL, 0.45 M). The vial was evacuated and backflushed with nitrogen. And the vial was capped and placed in an 60° C. bath. The reaction mixture was stirred at 60° C. for 4 h. The reaction mixture was cooled to room temperature. The reaction mixture was quenched by the addition of H₂O (100 mL). The mixture was extracted with EtOAc (3×100 mL), and the combined organic layers were washed with brine (2×100 mL). The combined organic layers were dried over Na₂SO₄, filtered and concentrated in vacuo. The resulting crude material was purified via silica gel chromatography to yield the desired product.

### Route 8:

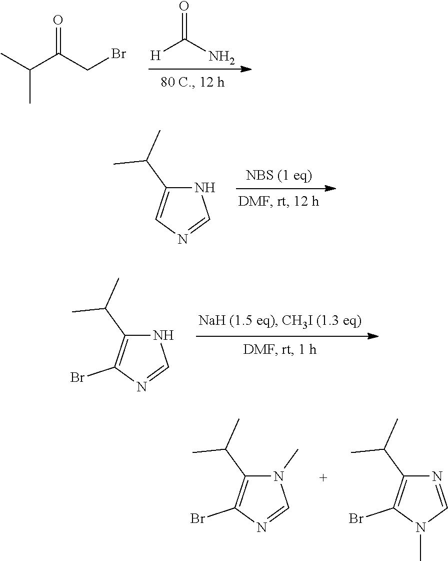

A 50 mL vial with stir bar was charged with 1-bromo-3-methylbutan-2-one (2.00 g, 12.12 mmol, 1.00 equiv.) and formamide (1 mL, 24.24 mmol, 2.00 equiv.). The flask was evacuated and flushed with nitrogen. The vial was capped and placed in an 80° C. bath. The reaction mixture was stirred at 80° C. for 12 h. The next morning, the reaction mixture was cooled to room temperature. The reaction mixture was quenched by the addition of H₂O (20 mL). The mixture was extracted with EtOAc (3×50 mL), and the combined organic layers were washed with brine (2×50 mL). The organic layer was then dried over Na₂SO₄, filtered and concentrated in vacuo. The resulting crude material was purified via silica gel chromatography to yield the desired product.

A 100 mL vial with stir bar was charged with 4-isopropyl-3H-imidazole (1.00 g, 9.08 mmol, 1.00 equiv.), NBS (1.78 g, 9.99 mmol, 1.10 equiv.) and DMF (20 mL, 0.45 M). The flask was evacuated and flushed with nitrogen. The vial was capped and placed in a 25° C. bath. The reaction mixture was stirred at 25° C. for 12 h. The next morning, the reaction mixture was quenched by H₂O (100 mL). The mixture was extracted with EtOAc (3×80 mL), and the combined organic layers were washed with brine (3×100 mL). The organic layer was then dried over Na₂SO₄, filtered and concentrated in vacuo. The resulting crude material was purified via silica gel chromatography to yield the desired product.

A 100 mL round bottom flask with stir bar was charged with 4-bromo-5-isopropyl-1H-imidazole (376.00 mg, 1.99 mmol, 1.00 equiv.) and DMF (10 mL, 0.20 M). NaH (60 wt % in mineral oil, 120 mg, 3.00 mmol, 1.51 equiv.) was slowly added, and the reaction mixture was allowed to stir at 0° C. for 20 min. The flask was evacuated and flushed with nitrogen. CH₃I (0.15 mL, 2.40 mmol, 1.21 equiv.) was added at 0° C., and the vial was capped and placed in an 25° C. bath. The reaction mixture was allowed to stir at 25° C. for 1 h. The reaction mixture was quenched by H₂O (50 mL). The mixture was extracted with EtOAc (4×50 mL), and the combined organic layers were washed with brine (3×100 mL). The combined organic layers were dried over Na₂SO₄, filtered and concentrated in vacuo. The resulting crude material was purified via silica gel chromatography to yield the desired product.

### Route 9:

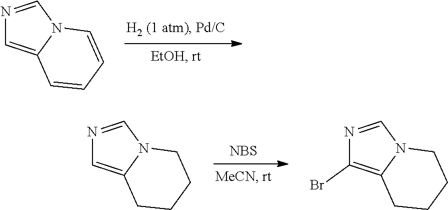

A 100 mL roundbottom flask with stir bar was charged with imidazo[1,5-a]pyridine (2.00 g, 16.9 mmol, 1.0 equiv.) and Pd/C (10 wt %, 1.80 g, 1.69 mmol, 0.1 equiv.). The flask was evacuated and backflushed with H2 (g). EtOH (20 mL, 0.9 M) was added, and the reaction mixture was stirred under 1 atm H2 overnight. The next morning, the reaction mixture was filtered through a plug of Celite and concentrated in vacuo. The crude material was carried on to the next step without further purification.

A 250 mL roundbottom flask was charged with 5,6,7,8-tetrahydroimidazo[1,5-a]pyridine (2.05 g, 16.8 mmol, 1.0 equiv.). MeCN (50 mL, 0.3 M) was added, and the reaction mixture was cooled to 0° C. NBS (3.29 g, 18.5 mmol, 1.1 equiv.) was slowly added, and the reaction mixture was allowed to warm to room temperature overnight. The next morning, the reaction mixture was filtered through a plug of Celite and concentrated in vacuo. The resulting crude material was purified via silica gel chromatography to yield the desired product.

### Route 10:

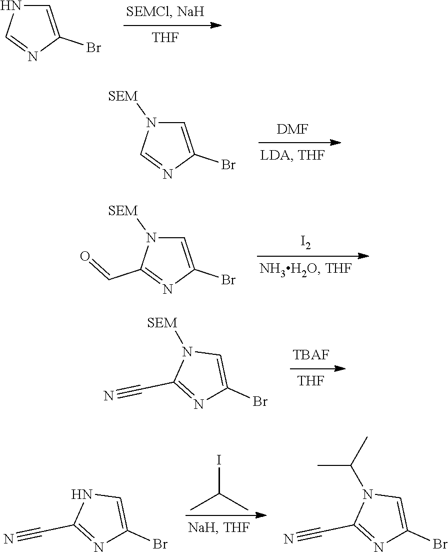

A 50 mL round bottom flask with stir bar was charged with 4-bromo-1H-imidazole (1.00 g, 6.80 mmol, 1.00 equiv.) and THF (15 mL, 0.45 M). NaH (60 wt % in mineral oil, 680.40 mg, 17.01 mmol, 2.50 equiv) was slowly added, and the reaction mixture was allowed to stir at 0° C. for 20 min. SEMCl (1.70 g, 10.21 mmol, 1.50 equiv.) was added at 0° C., and the vial was capped and placed in an 25° C. bath. The reaction mixture was allowed to stir at 25° C. for 2 h. The reaction mixture was quenched by the addition of H₂O (50 mL). The mixture was extracted with DCM (3×50 mL), and the combined organic layers were washed with brine (1×50 mL). The combined organic layers were dried over Na₂SO₄, filtered and concentrated in vacuo. The resulting crude material was purified via RP chromatography to yield the desired product.

A 100 mL vial with stir bar was charged with 4-bromo-1-{[2-(trimethylsilyl)ethoxy]methyl}imidazole (2.00 g, 7.21 mmol, 1.00 equiv.) and THF (30 mL, 0.2 M). The flask was evacuated and flushed with nitrogen. LDA (2 M in THF, 18.04 mL, 36.07 mmol, 5.00 equiv.) was added dropwise over 5 min at 0° C., and the mixture was stirred for 30 min at 0° C. DMF (790 mg, 10.82 mmol, 1.50 equiv.) in dry THF (10 mL, 0.18 M) was added dropwise over 5 min at 0° C., and the vial was capped and placed in a 0° C. bath. The reaction mixture was stirred at 0° C. for 8 h. The reaction mixture was quenched by the addition of sat. NH₄Cl (aq.) (80 mL). The mixture was extracted with EtOAc (3×80 mL), and the combined organic layers were washed with brine (2×80 mL). The organic layer was then dried over Na₂SO₄, filtered and concentrated in vacuo. The resulting crude material was purified via silica gel chromatography to yield the desired product.

A 100 mL vial with stir bar was charged with 4-bromo-1-{[2-(trimethylsilyl)ethoxy]methyl}imidazole-2-carbaldehyde (1.00 g, 3.28 mmol, 1.00 equiv.) and THF (10 mL, 0.33 M). NH₃·H₂O (27% in water, 20 mL, 289.05 mmol, 88.13 equiv.) and iodine (1.25 g, 4.91 mmol, 1.50 equiv.) were added, and the vial was capped and placed in an 25° C. bath. The reaction mixture was stirred at 25° C. for 1 h. The reaction mixture was quenched by the addition of H₂O (20 mL). The mixture was extracted with DCM (3×50 mL), and the combined organic layers were washed with brine (2×50 mL). The organic layer was then dried over Na₂SO₄, filtered and concentrated in vacuo. The crude product was used in the next step without further purification.

A 100 mL vial with stir bar was charged with 4-bromo-1-{[2-(trimethylsilyl)ethoxy]methyl}imidazole-2-carbonitrile (1.00 g, 3.31 mmol, 1.00 equiv.) and THF (10 mL, 0.33 M). TBAF (1 M in THF, 33.1 mL, 33.1 mmol, 10.00 equiv.) was added, and the vial was capped and placed in an 70° C. bath. The reaction mixture was stirred at 70° C. for 4 h. The reaction mixture was cooled to room temperature. The reaction mixture was quenched by the addition of H₂O (80 mL). The mixture was extracted with DCM (3×100 mL), and the combined organic layers were washed with brine (1×80 mL). The organic layer was then dried over Na₂SO₄, filtered and concentrated in vacuo. The resulting crude material was purified via RP chromatography to yield the desired product.

The alkylation was performed as described in route 8.

### Route 11:

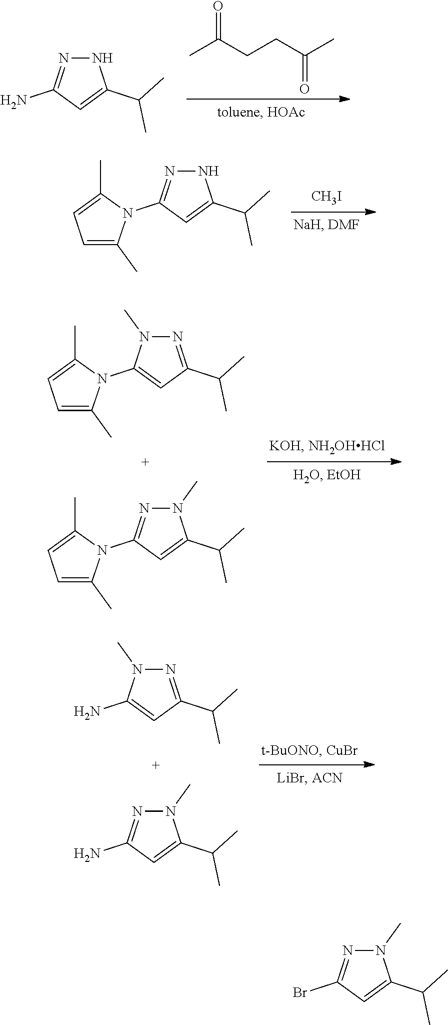

A 50 mL vial with stir bar was charged with 5-isopropyl-1H-pyrazol-3-amine (500 mg, 3.99 mmol, 1.00 equiv.), 2,5-hexanedione (600 mg, 5.26 mmol, 1.32 equiv.) and toluene (10 mL, 0.4 M). AcOH (0.3 mL, 5.25 mmol, 1.31 equiv.) was added. The flask was evacuated and flushed with nitrogen. The vial was capped and placed in a 120° C. bath. The reaction mixture was stirred at 120° C. overnight. The next morning, the reaction mixture was cooled to room temperature. The reaction mixture was concentrated in vacuo. The resulting material was charged with H₂O (50 mL). The mixture was extracted with DCM (3×50 mL), and the combined organic layers were washed with brine (2×40 mL). The combined organic layers were dried over Na₂SO₄, filtered and concentrated in vacuo. The resulting crude material was purified via silica gel chromatography to yield the desired product.

A 100 mL round bottom flask with stir bar was charged with 3-(2,5-dimethylpyrrol-1-yl)-5-isopropyl-1H-pyrazole (1.02 g, 5.02 mmol, 1.00 equiv.) and THF (10 mL, 0.50 M). NaH (60 wt % in mineral oil, 301.20 mg, 7.53 mmol, 1.50 equiv.) was slowly added, and the reaction mixture was allowed to stir at 0° C. for 20 min. The flask was evacuated and flushed with nitrogen. CH₃I (0.375 mL, 6.02 mmol, 1.20 equiv.) was added at 0° C., and the vial was capped and placed in an 25° C. bath. The reaction mixture was allowed to stir at 25° C. for 2 h. The reaction mixture was quenched by the addition of H₂O (50 mL). The mixture was extracted with DCM (3×50 mL), and the combined organic layers were washed with brine (2×50 mL). The combined organic layers were dried over Na₂SO₄, filtered and concentrated in vacuo. The resulting crude material was purified via silica gel chromatography to yield the desired product.

A 100 mL round bottom flask with stir bar was charged with hydroxylamine hydrochloride (1.92 g, 27.61 mmol, 6.00 equiv.) and EtOH (10 mL, 2.8 M). A solution of potassium hydroxide (770 mg, 13.81 mmol, 3.00 equiv.) in water (10 mL) and EtOH (10 mL, 0.15 M) were slowly added, followed by 3-(2,5-dimethylpyrrol-1-yl)-5-isopropyl-1-methylpyrazole (1.00 g, 4.60 mmol, 1.00 equiv.). The flask was evacuated and flushed with nitrogen, and the vial was capped and placed in an 80° C. bath. The reaction mixture was allowed to stir at 80° C. for 12 h. The reaction mixture was cooled to room temperature. The reaction mixture was quenched by the addition of H₂O (50 mL). The mixture was extracted with EtOAc (3×50 mL), and the combined organic layers were washed with brine (2×50 mL). The combined organic layers were dried over Na₂SO₄, filtered and concentrated in vacuo. The resulting crude material was purified via silica gel chromatography to yield the desired product.

A 250 mL round bottom flask with stir bar was charged with t-BuNO₂ (1.75 g, 16.97 mmol, 1.52 equiv.) CuBr (2.41 g, 16.80 mmol, 1.51 equiv.), LiBr (1.25 g, 14.39 mmol, 1.30 equiv.) and MeCN (80 mL). After 10 min, this mixture was added to a flask containing a suspension of the 5-isopropyl-1-methyl-1H-pyrazol-3-amine (1.55 g, 11.14 mmol, 1.00 equiv.) in MeCN (20 mL, 0.11 M). The flask was evacuated and flushed with nitrogen. The vial was capped and placed in an 50° C. bath. The reaction mixture was allowed to stir at 50° C. for 12 h. The next morning, the reaction mixture was cooled to room temperature. The reaction mixture was poured into EtOAc (300 mL), washed with NaHCO₃ (1×150 mL), followed by brine (2×150 mL). The combined organic layers were dried over Na₂SO₄, filtered and concentrated in vacuo. The resulting crude material was purified via silica gel chromatography to yield the desired product.

### Route 12:

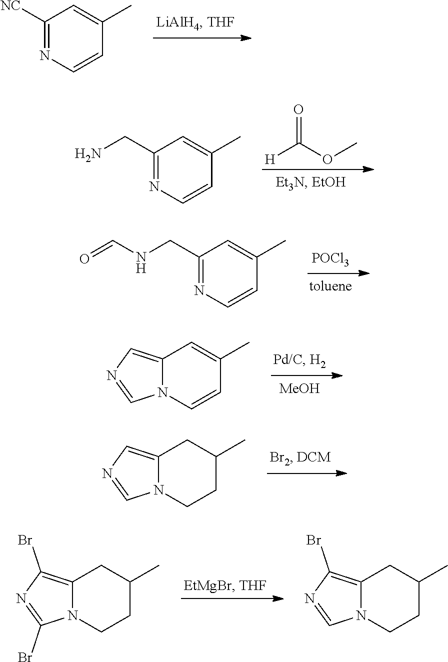

A 100 mL vial with stir bar was charged with 4-methylpicolinonitrile (1.00 g, 8.47 mmol, 1.00 equiv.) and THF (15 mL, 0.56 M). LiAlH₄ (642.53 mg, 16.93 mmol, 2.00 equiv.) was slowly added at 0° C. The flask was evacuated and flushed with nitrogen. The vial was capped and placed in an 25° C. bath. The reaction mixture was stirred at 25° C. for 1 h. The reaction mixture was quenched by the addition of H₂O (0.6 mL) and NaOH (aq) (15% in water, 0.6 mL). The solids were filtered out. The filter cake was washed with EtOAc (3×50 mL). The combined filtrate was concentrated in vacuo. The crude product was used in the next step without further purification.

A 100 mL vial with stir bar was charged with (4-methylpyridin-2-yl)methanamine (1.00 g, 8.19 mmol, 1.00 equiv.) and EtOH (15 mL, 0.55 M). Methyl formate (983.08 mg, 16.37 mmol, 2.00 equiv.) and Et3N (2.3 mL, 16.37 mmol, 2.00 equiv.) were added. The flask was evacuated and flushed with nitrogen. The vial was capped and placed in an 60° C. bath. The reaction mixture was stirred at 60° C. for 12 h. The next morning, the reaction mixture was cooled to room temperature. The resulting solution was concentrated in vacuo. The resulting crude material was purified via silica gel chromatography to yield the desired product.

A 100 mL vial with stir bar was charged with N-[(4-methylpyridin-2-yl)methyl]formamide (500.00 mg, 3.33 mmol, 1.00 equiv.) and toluene (10 mL, 0.3 M). POCl₃ (1.02 g, 6.66 mmol, 2.00 equiv.) was added. The flask was evacuated and flushed with nitrogen. The vial was capped and placed in a 90° C. bath. The reaction mixture was stirred at 90° C. for 1 h. The reaction mixture was cooled to room temperature. The resulting solution was concentrated in vacuo. The resulting material was charged with sat. NaHCO₃ (aq.) (20 mL). The mixture was extracted with EtOAc (3×40 mL), and the combined organic layers were washed with brine (2×40 mL). The organic layer was then dried over Na2SO4, filtered and concentrated in vacuo. The resulting crude material was purified via silica gel chromatography to yield the desired product.

The reduction of 7-methylimidazo[1,5-a]pyridine was performed as described in route 9.

A 100 mL vial with stir bar was charged with 7-methyl-5,6,7,8-tetrahydroimidazo[1,5-a]pyridine (1.00 g, 7.34 mmol, 1.00 equiv.) and DCM (20 mL, 0.37 M). Br2 (2.35 g, 14.68 mmol, 2.00 equiv.) was added at 0° C. The vial was capped and placed in an 0° C. bath. The reaction mixture was stirred at 0° C. for 1 h. The reaction mixture was warmed to room temperature. The reaction mixture was quenched by NaHCO₃ (s). The solids were filtered out. The filter cake was washed with DCM (2×20 mL). The combined filtrate was concentrated in vacuo. The crude product was used in the next step without further purification.

A 50 mL vial with stir bar was charged with 1,3-dibromo-7-methyl-5,6,7,8-tetrahydroimidazo[1,5-a]pyridine (500.00 mg, 1.70 mmol, 1.00 equiv.) and THF (10 mL, 0.17 M). EtMgBr (2.00 M in THF, 1.70 mL, 3.40 mmol, 2.00 equiv.) was added at 0° C. The vial was capped and placed in a 25° C. bath. The reaction mixture was stirred at 25° C. for 1 h. The reaction mixture was quenched by sat. NH4Cl (aq.) (20 mL). The mixture was extracted with DCM (3×40 mL), and the combined organic layers were washed with brine (1×40 mL). The organic layer was then dried over Na2SO4, filtered and concentrated in vacuo. The resulting crude material was purified via silica gel chromatography to yield the desired product.

The following compounds were prepared via a similar method:

| | Compound name |
|---|---|
| B98 | 1-bromo-6-methyl-5,6,7,8-tetrahydroimidazo[1,5-a]pyridine |
| B101 | 1-bromo-5-methyl-5,6,7,8-tetrahydroimidazo[1,5-a]pyridine |
| B102 | 1-bromo-8-methyl-5,6,7,8-tetrahydroimidazo[1,5-a]pyridine |

### Route 13:

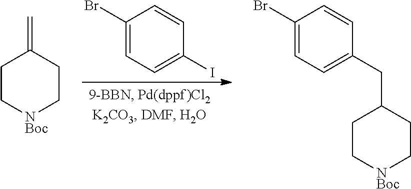 [SMILES: C=C1CCN(C(=O)OC(C)(C)C)CC1 ; Brc1ccc(I)cc1 ; CC(C)(C)OC(=O)N1CCC(Cc2ccc(Br)cc2)CC1]

A 50 mL vial with stir bar was charged with tert-butyl 4-methylidenepiperidine-1-carboxylate (2.00 g, 10.14 mmol, 1.00 equiv.) and 9-BBN (0.5 M in THF, 20 mL, 20 mmol, 1.97 equiv.). The vial was evacuated and backflushed with nitrogen the resulting solution was refluxed for 1 h. And then the reaction mixture was cooled to room temperature. 4-bromoiodobenzene (2.58 g, 9.12 mmol, 0.90 equiv.), Pd(dppf)Cl₂ (740 mg, 1.01 mmol, 0.10 equiv.), K₂CO₃ (1.82 g, 13.18 mmol, 1.30 equiv.), DMF (25.0 mL, 0.34 M) and H₂O (5 mL) were added. The vial was capped and placed in an 60° C. bath. The reaction mixture was stirred at 60° C. for 3 h. The reaction mixture was cooled to room temperature, the mixture was poured into EtOAc (200 mL) and washed with brine (3×100 mL). The combined organic layers were dried over Na₂SO₄, filtered and concentrated in vacuo. The resulting crude material was purified via silica gel chromatography to yield the desired product.

### Route 14:

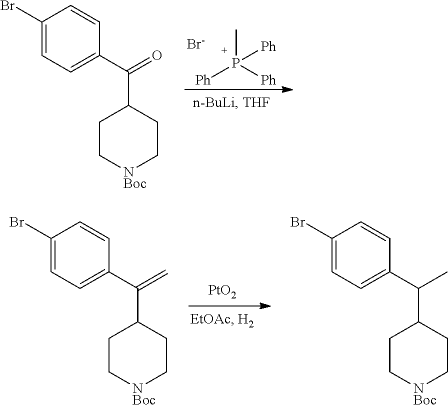

A 100 mL vial with stir bar was charged with methyltriphenylphosphonium bromide (3.88 g, 10.86 mmol, 2.00 equiv.) and THF (20 mL, 0.5 M). The flask was evacuated and flushed with nitrogen. n-BuLi (2.50 M in hexanes, 4.34 mL, 10.86 mmol, 2.00 equiv.) was added dropwise over 5 min at −78° C., and the mixture was stirred for 15 min at −78° C. The mixture was then warmed to 0° C. and then cooled back to −78° C. Tert-butyl 4-(4-bromobenzoyl)piperidine-1-carboxylate (2.00 g, 5.43 mmol, 1.00 equiv.) in dry THF (10 mL, 0.18 M) was added dropwise over 5 min at −78° C. The reaction was allowed to stir at −78° C. for 15 min. After this time, the solution was warmed to 25° C., and the vial was capped and placed in a 25° C. bath. The reaction mixture was stirred at 25° C. overnight. The next morning, the reaction mixture was quenched by the addition of H₂O (100 mL). The mixture was extracted with DCM (3×100 mL), and the combined organic layers were washed with brine (2×80 mL). The organic layer was dried over Na₂SO₄, filtered and concentrated in vacuo. The resulting crude material was purified via silica gel chromatography to yield the desired product.

A 50 mL vial with stir bar was charged with tert-butyl 4-(1-(4-bromophenyl)vinyl)piperidine-1-carboxylate (500 mg, 1.37 mmol, 1.00 equiv.), PtO₂ (61.99 mg, 0.27 mmol, 0.20 equiv.) and EtOAc (8.00 mL, 0.17 M) under nitrogen atmosphere. The flask was evacuated and flushed with hydrogen. The reaction mixture was hydrogenated at room temperature for 12 hours under 1 atm hydrogen using a hydrogen balloon. Then the reaction mixture was filtered through a celite pad and the filtrate was concentrated under reduced pressure. The crude product was used in the next step without further purification.

### Route 15:

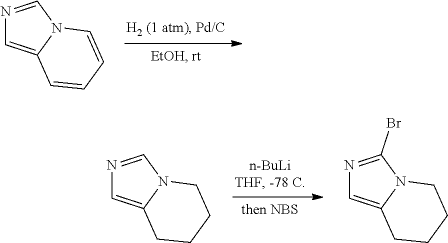 [SMILES: c1ccn2cncc2c1 ; c1ncn2c1CCCC2 ; "n-BuLiTHF, -78 C." ; Brc1ncc2n1CCCC2]

The reduction was performed as described in route 9.

A flame-dried 100 mL roundbottom flask with stir bar was charged with 5,6,7,8-tetrahydroimidazo[1,5-a]pyridine (1.3 g, 11 mmol, 1.5 equiv.), evacuated and backflushed with nitrogen. Dry THF (30 mL, 0.3 M) was added, and the reaction mixture was cooled to −78° C. n-BuLi (2.5 M in hexanes, 4.3 mL, 11 mmol, 1.5 equiv.) was added at −78° C. The reaction mixture was allowed to warm to 0° C. over 30 min. After 30 min, N-bromosuccinimide (1.3 g, 7.1 mmol, 1.0 equiv.) was added portion-wise, and the reaction mixture was allowed to warm to room temperature overnight. The next morning, the reaction mixture was quenched with water (5 mL) and filtered through a plug of Celite. The resulting solution was concentrated in vacuo, and the crude material was purified via silica gel chromatography to yield the desired product.

### Route 16:

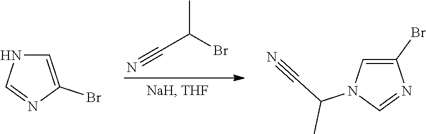

A 100 mL round bottom flask with stir bar was charged with 4-bromo-1H-imidazole (1.00 g, 6.80 mmol, 1.00 equiv.) and THF (15 mL, 0.45 M). NaH (60 wt % in mineral oil, 408.4 mg, 10.21 mmol, 1.50 equiv.) was slowly added at 0° C., and the reaction mixture was allowed to stir at 0° C. for 20 min. 2-bromopropanenitrile (1.50 g, 11.11 mmol, 1.63 equiv.) was added at 0° C., and the vial was capped and placed in an 50° C. bath. The reaction mixture was allowed to stir at 50° C. for 4 h. The reaction mixture was cooled to room temperature. The reaction mixture was quenched by the addition of H₂O (5 mL). The resulting solution was concentrated in vacuo. The resulting crude material was purified via silica gel chromatography to yield the desired product. The desired isomer was confirmed by NOESY spectroscopy.

The following compounds were prepared via a similar method:

| | | Leaving |
|---|---|---|
| | Compound name | Group used |
| B112 | 2-(4-bromo-1H-imidazol-1- | Br used |
| and B115 | yl)acetonitrile | |
| B122 | 4-bromo-1-(2,2,2-trifluoroethyl)- | OTs used |
| | 1H-imidazole | |
| B123 | 4-bromo-1-(oxetan-3-yl)-1H-imidazole | I used |
| B124 | 4-bromo-1-(tetrahydrofuran-3-yl)- | I used |
| | 1H-imidazole | |
| B127 | 4-bromo-1-cyclobutyl-1H-imidazole | Br used |

Benzyl Bromide Syntheses

### Route 1:

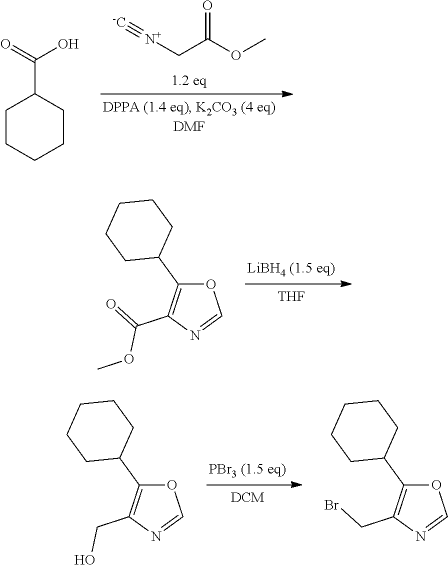

A 250 mL vial with stir bar was charged with cyclohexanecarboxylic acid (5.40 g, 42.13 mmol, 1.00 equiv.), K₂CO₃ (23.30 g, 168.59 mmol, 4.00 equiv.) and DMF (100 mL, 0.42 M). DPPA (16.10 g, 58.50 mmol, 1.39 equiv.) and methyl 2-isocyanoacetate (5.00 g, 50.46 mmol, 1.20 equiv.) were added at 0° C., and the vial was capped and placed in an 25° C. bath. The reaction mixture was stirred at 25° C. overnight. The next morning, the reaction was quenched by the addition of water (300 mL). The resulting solution was extracted with EtOAc (3×250 mL), and the combined organic layers were washed with brine (3×300 mL). The organic layer was dried over Na2SO4, filtered and concentrated in vacuo. The resulting crude material was purified via silica gel chromatography to yield the desired product.

A 100 mL vial with stir bar was charged with 5-cyclohexyl-1,3-oxazole-4-carboxylate (2.24 g, 10.71 mmol, 1.00 equiv.) and THE (20 mL, 0.54 M). LiBH4 (349.80 mg, 16.06 mmol, 1.50 equiv.) was added at 0° C., and the vial was capped and placed in an 25° C. bath. The reaction mixture was stirred at 25° C. overnight. The next morning, the reaction was then quenched by the addition of water (50 mL). The pH of the solution was adjusted to 6 with 1 M HCl (aq.). The resulting solution was extracted with EtOAc (3×100 mL). The organic layer was dried over Na₂SO₄, filtered and concentrated in vacuo. The resulting crude material was purified via RP chromatography to yield the desired product.

A 100 mL vial with stir bar was charged with (5-cyclohexyl-1,3-oxazol-4-yl)methanol (1.28 g, 7.06 mmol, 1.00 equiv.) and DCM (20.00 mL, 0.35 M). Phosphorus tribromide (1.0 mL, 10.46 mmol, 1.50 equiv.) was added at 0° C., and the vial was capped and placed in an 0° C. bath. The reaction mixture was stirred at 0° C. for 1 h. The pH of the solution was adjusted to 8 with sat. NaHCO₃ (aq.). The resulting solution was extracted with EtOAc (3×50 mL), and the combined organic layers were washed with brine (1×50 mL). The organic layer was dried over Na₂SO₄, filtered and concentrated in vacuo. The crude product was used in the next step without further purification.

The following compounds were prepared via a similar method:

| | Compound name |
|---|---|
| B125 | 4-(bromomethyl)-5-isopropyloxazole |
| B126 | 4-(bromomethyl)-5-ethyloxazole |
| B130 | 4-(bromomethyl)-5-(methoxymethyl)oxazole |
| B131 | 4-(bromomethyl)-5-(tert-butyl)oxazole |
| B132 | 4-(bromomethyl)-5-cyclopropyloxazole |
| B133 | 4-(bromomethyl)-5-cyclobutyloxazole |

### Route 2:

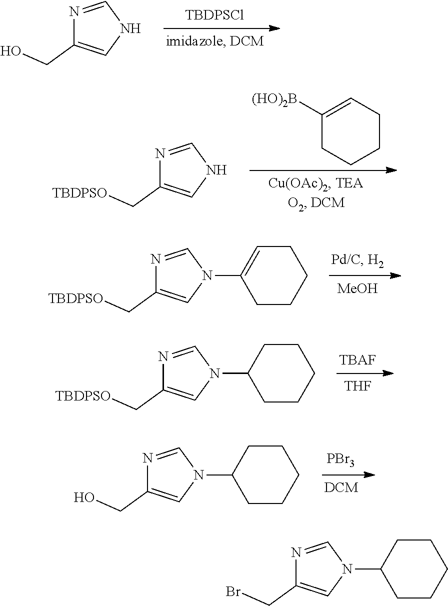

A 250 mL vial with stir bar was charged with 4-(hydroxymethyl)imidazole (2.00 g, 20.39 mmol, 1.00 equiv.) and DCM (100 mL, 0.20 M). TBDPSCl (8.41 g, 30.58 mmol, 1.50 equiv.) and imidazole (2.78 g, 40.77 mmol, 2.00 equiv.) were added, and the vial was capped and placed in an 25° C. bath. The reaction mixture was stirred at 25° C. overnight. The next morning, the reaction mixture was poured into DCM (300 mL) and washed with H₂O (1×200 mL), followed by brine (2×200 mL). The organic layer was then dried over Na₂SO₄, filtered and concentrated in vacuo. The resulting crude material was purified via silica gel chromatography to yield the desired product.

A 100 mL vial with stir bar was charged with 4-[[(tert-butyldiphenylsilyl)oxy]methyl]-1H-imidazole (3.00 g, 8.92 mmol, 1.00 equiv.), cyclohex-1-en-1-ylboronic acid (5.61 g, 44.58 mmol, 5.00 equiv.), Cu(OAc)₂ (4.05 g, 22.29 mmol, 2.50 equiv.), TEA (3.7 mL, 26.75 mmol, 3.00 equiv.) and DCM (120 mL, 0.07 M) under nitrogen atmosphere. The flask was evacuated and flushed with oxygen. The reaction mixture was stirred at room temperature for 24 h under oxygen atmosphere using an oxygen balloon. The reaction mixture was poured into DCM (300 mL), quenched by the addition of NH3·H₂O (30 mL), and washed with H₂O (1×150 mL) and brine (3×150 mL). The organic layer was dried over Na₂SO₄, filtered and concentrated in vacuo. The resulting crude material was purified via silica gel chromatography to yield the desired product.

A 100 mL vial with stir bar was charged with 4-[[(tert-butyldiphenylsilyl)oxy]methyl]-1-(cyclohex-1-en-1-yl)imidazole (3.00 g, 7.20 mmol, 1.00 equiv.), Pd/C (10 wt %, 3.00 g, 28.20 mmol, 3.92 equiv.) and MeOH (40 mL, 0.18 M) under nitrogen atmosphere. The flask was evacuated and flushed with hydrogen. The reaction mixture was hydrogenated at room temperature for 3 hours under hydrogen atmosphere using a hydrogen balloon. Then the reaction mixture was filtered through a celite pad and the filtrate was concentrated under reduced pressure. The resulting crude material was purified via RP chromatography to yield the desired product.

A 50 mL vial with stir bar was charged with 4-[[(tert-butyldiphenylsilyl)oxy]methyl]-1-cyclohexylimidazole (2.50 g, 5.97 mmol, 1.00 equiv.), TBAF hydrate (3.12 g, 11.94 mmol, 2.00 equiv.) and THF (40 mL, 0.15 M). The vial was capped and placed in a 25° C. bath. The reaction mixture was stirred at 25° C. for 2 h. The resulting mixture was concentrated in vacuo. The resulting crude material was purified via silica gel chromatography to yield the desired product.

The bromide was installed as described in route 1.

### Route 3:

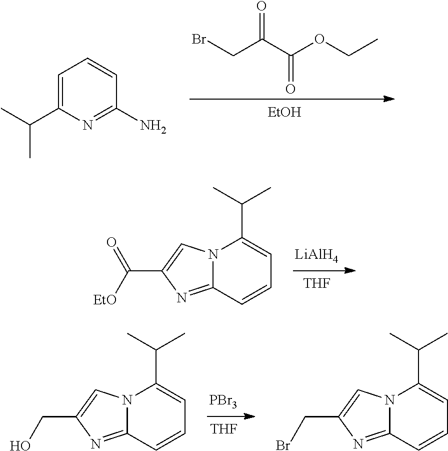

A 100 mL vial with stir bar was charged with 6-isopropylpyridin-2-amine (670.00 mg, 1.73 mmol, 1.00 equiv.), ethyl 3-bromo-2-oxopropanoate (655.00 mg, 8.617 mmol, 5.00 equiv.) and EtOH (10 mL, 0.17 M), and the vial was capped and placed in an 80° C. bath. The reaction mixture was stirred at 80° C. overnight. The next morning, the reaction mixture was cooled to room temperature. The reaction mixture was concentrated in vacuo. The resulting material was charged with H₂O (30 mL). The mixture was extracted with EtOAc (3×40 mL), and the combined organic layers were washed with brine (1×50 mL). The combined organic layers were dried over Na₂SO₄, filtered and concentrated in vacuo. The resulting crude material was purified via silica gel chromatography to yield the desired product.

A 100 mL vial with stir bar was charged with ethyl 5-isopropylimidazo[1,2-a]pyridine-2-carboxylate (1.20 g, 5.17 mmol, 1.00 equiv.) and THF (20 mL, 0.26 M). LiAlH₄ (392.15 mg, 10.33 mmol, 2.00 equiv.) was slowly added at 0° C. The flask was evacuated and flushed with nitrogen. The vial was capped and placed in an 25° C. bath. The reaction mixture was stirred at 25° C. for 1 h. The reaction mixture was quenched by H₂O (2 mL) and NaOH (15% in water, 0.4 mL). The solids were filtered out. The filter cake was washed with EtOAc (4×50 mL). The combined filtrate was concentrated under vacuum. The crude product was used in the next step without further purification.

A 100 mL vial with stir bar was charged with {5-isopropylimidazo[1,2-a]pyridin-2-yl}methanol (1.00 g, 5.26 mmol, 1.00 equiv.) and DCM (20 mL, 0.26 M). PBr3 (1.0 mL, 10.51 mmol, 2.00 equiv.) was slowly added at 0° C. The flask was evacuated and flushed with nitrogen. The vial was capped and placed in a 25° C. bath. The reaction mixture was stirred at 25° C. for 1 h. The reaction mixture was quenched by the addition of NaHCO₃ (s). The solids were filtered out. The filter cake was washed with DCM (50 mL). The combined filtrate was concentrated in vacuo. The crude product was used in the next step without further purification.

The following compounds were prepared via a similar method:

| | Compound name |
|---|---|
| B78 and B79 | 2-(bromomethyl)-5-ethylimidazo[1,2-a]pyridine |

### Route 4:

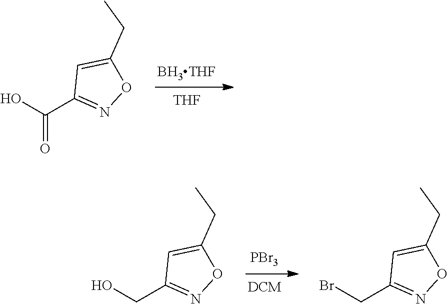

A 100 mL vial with stir bar was charged with 5-ethylisoxazole-3-carboxylic acid (1.50 g, 10.63 mmol, 1.00 equiv.) and THF (30 mL, 0.35 M). BH₃·THF (1 M in THF, 53.15 mL, 53.15 mmol, 5.00 equiv.) was slowly added at 0° C. And the vial was capped and placed in an 25° C. bath. The reaction mixture was stirred at 25° C. for 1 h. The reaction mixture was quenched by the addition of H₂O (100 mL). The mixture was extracted with DCM (3×40 mL), and the combined organic layers were washed with brine (1×30 mL). The organic layer was then dried over Na₂SO₄, filtered and concentrated in vacuo. The resulting crude material was purified via silica gel chromatography to yield the desired product.

The bromide was installed as described in route 1.

The following compounds were prepared via a similar method:

| | Compound name |
|---|---|
| B90 | 3-(bromomethyl)-5-isopropylisoxazole |

### Route 5:

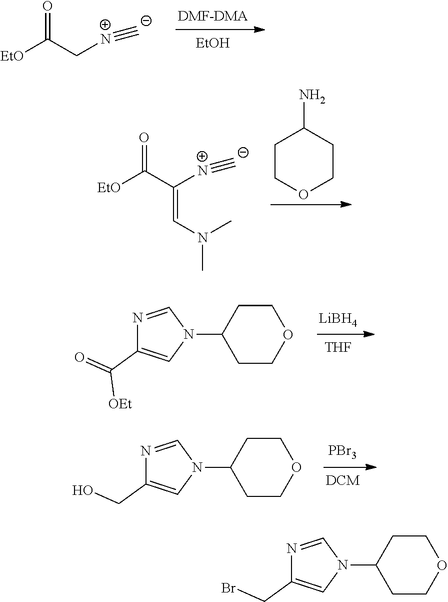

A 100 mL vial with stir bar was charged with ethyl 2-isocyanoacetate (1.50 g, 13.26 mmol, 1.00 equiv.), N,N-dimethylformamide dimethyl acetal (610.00 mg, 26.52 mmol, 2.00 equiv.) and EtOH (20 mL, 0.66 M). The flask was evacuated and flushed with nitrogen. The vial was capped and placed in a 25° C. bath. The reaction mixture was stirred at 25° C. overnight. The resulting solution was concentrated in vacuo. The crude product was used in the next step without further purification.

A 50 mL vial with stir bar was charged with ethyl (Z)-3-(dimethylamino)-2-isocyanoacrylate (1.50 g, 8.92 mmol, 1.00 equiv.) and 4-aminotetrahydropyran (1.1 mL, 10.70 mmol, 1.20 equiv.). The flask was evacuated and flushed with nitrogen. The vial was capped and placed in a 70° C. bath. The reaction mixture was stirred at 70° C. overnight. The next morning, the reaction mixture was cooled to room temperature. The resulting solution was concentrated in vacuo. The resulting material was charged with H₂O (20 mL). The mixture was extracted with DCM (3×40 mL), and the combined organic layers were washed with brine (1×40 mL). The organic layer was then dried over Na₂SO₄, filtered and concentrated in vacuo. The resulting crude material was purified via silica gel chromatography to yield the desired product.

The ester reduction was performed as described in route 1.

The bromination was performed as described in route 1.

### Biological Assays

### Dox-Induced PD1-ss-Gluc Assay

Flp-In 293 T-REx™ cells were transfected with pcDNA™5/FRT/TO plasmid inserted with cDNA encoding Gaussia Luciferase fused to the 3′ end of cDNA encoding PD1 signal sequence plus 10 amino acids (N-MQIPQAPWPWWAVLQLGWRPGWFLDSPDR-C) (SEQ ID NO: 1). Transfected cells were selected for resistance to the selectable markers Hygromycin and Blasticidin to create a stable cell line that contained the PD1-ss+10aa/Gaussia Luciferase cDNA insert whose expression was regulated under the T-REx™ system. The day before assay, cells were trypsinized and plated in 384-well tissue culture plates. The next day, compound dilutions in DMSO/media containing doxycycline were added to the wells and incubated at 37° C., 5% CO₂. 24 hours later, coelenterazine substrate was added to each well and luciferase signal was quantified using Tecan Infinite M1000 Pro for potency determination.

Results for select compounds provided herein are shown in the Tables below. For chemical structures that include one or more stereoisomers, but are illustrated without indicating stereochemistry, the assay data refers to a mixture of stereoisomers.

### Dox Induced TNFα-FL-Gluc Assay

Flp-In 293 T-REx™ cells were transfected with pcDNA™5/FRT/TO plasmid inserted with cDNA encoding Gaussia Luciferase fused to the 3′ end of cDNA encoding full length TNFα (amino acids 1-233). Transfected cells were selected for resistance to the selectable markers Hygromycin and Blasticidin to create a stable cell line that contained the TNFα-FL/Gaussia Luciferase cDNA insert whose expression was regulated under the T-REx™ system. The day before assay, cells were trypsinized and plated in 384-well tissue culture plates. The next day, compound dilutions in DMSO/media containing doxycycline were added to the wells and incubated at 37° C., 5% CO₂. 24 hours later, coelenterazine substrate was added to each well and luciferase signal was quantified using Tecan Infinite M1000 Pro for potency determination.

Results for select compounds provided herein are shown in the Tables below. For chemical structures that include one or more stereoisomers, but are illustrated without indicating stereochemistry, the assay data refers to a mixture of stereoisomers.

### Dox-Induced Her3-ss-Gluc Assay

Flp-In 293 T-REx™ cells were transfected with pcDNA™5/FRT/TO plasmid inserted with cDNA encoding Gaussia Luciferase fused to the 3′ end of cDNA encoding HER3 signal sequence plus 4 amino acids (N-MRANDALQVLGLLFSLARGSEVG-C) (SEQ ID NO: 2). Transfected cells were selected for resistance to the selectable markers Hygromycin and Blasticidin to create a stable cell line that contained the HER3-ss+4aa/Gaussia Luciferase cDNA insert whose expression was regulated under the T-REx™ system. The day before assay, cells were trypsinized and plated in 384-well tissue culture plates. The next day, compound dilutions in DMSO/media containing doxycycline were added to the wells and incubated at 37° C., 5% CO₂. 24 hours later, coelenterazine substrate was added to each well and luciferase signal was quantified using Tecan Infinite M1000 Pro for potency determination.

Results for select compounds provided herein are shown in the Tables below. For chemical structures that include one or more stereoisomers, but are illustrated without indicating stereochemistry, the assay data refers to a mixture of stereoisomers.

### Dox Induced IL2-FL-Gluc Assay

Flp-In 293 T-REx™ cells were transfected with pcDNA™5/FRT/TO plasmid inserted with cDNA encoding Gaussia Luciferase fused to the 3′ end of cDNA encoding full length IL-2 (amino acids 1-153). Transfected cells were selected for resistance to the selectable markers Hygromycin and Blasticidin to create a stable cell line that contained the IL-2-FL/Gaussia Luciferase cDNA insert whose expression was regulated under the T-REx™ system. The day before assay, cells were trypsinized and plated in 384-well tissue culture plates. The next day, compound dilutions in DMSO/media containing doxycycline were added to the wells and incubated at 37° C., 5% CO₂. 24 hours later, coelenterazine substrate was added to each well and luciferase signal was quantified using Tecan Infinite M1000 Pro for potency determination.

Results for select compounds provided herein are shown in the Tables below. For chemical structures that include one or more stereoisomers, but are illustrated without indicating stereochemistry, the assay data refers to a mixture of stereoisomers.

### H929 Cell Viability Assay

The human multiple myeloma cell line NCI-H929 was cultured in Advanced RPMI 1640 media (Gibco®) supplemented with 6% fetal bovine serum, 2 mM Glutamine, and 1× Penicillin/Streptomycin. On the day of assay, cells were resuspended in RPMI 1640 media supplemented with 10% fetal bovine serum, 2 mM Glutamine, and 1× Penicillin/Streptomycin and plated in 384-well tissue culture plates and treated with compound dilutions in DMSO/media. Plates were incubated at 37° C., 5% CO₂ for 48 hours. After 48 hours, Celltiter-Glo® (Promega) was added to each well and luciferase signal was quantified using Tecan Infinite M1000 Pro for cell viability determination.

Results for select compounds provided herein are shown in the Tables below. For chemical structures that include one or more stereoisomers, but are illustrated without indicating stereochemistry, the assay data refers to a mixture of stereoisomers.

### U266 Cell Viability Assay

The human multiple myeloma cell line U266B1 was cultured in RPMI 1640 media supplemented with 10% fetal bovine serum, 2 mM Glutamine, and 1× Penicillin/Streptomycin. Cells were plated in 384-well tissue culture plates and treated with compound dilutions in DMSO/media. Plates were incubated at 37° C., 5% CO₂ for 48 hours. After 48 hours, Celltiter-Glo® (Promega) was added to each well and luciferase signal was quantified using Tecan Infinite M1000 Pro for cell viability determination.

Results for select compounds provided herein are shown in the Tables below. For chemical structures that include one or more stereoisomers, but are illustrated without indicating stereochemistry, the assay data refers to a mixture of stereoisomers.

### Liver Microsome Stability Assays

Stability of a compound was assessed in the presence of liver microsomes from various sources—mouse, rat, monkey and human liver microsomes. 1.0 uM compound, 0.4% DMSO in 0.1 M Potassium Phosphate with 1.0 mg/mL liver microsomes, were incubated at 37° C. with or without 1 mM NADPH. The samples were quenched at 0, and 30 minutes.

TABLE 1

| | 24 hr Dox | 24 hr Dox | 24 hr Dox | 24 hr Dox | 24 hr Dox | 24 hr Dox |
|---|---|---|---|---|---|---|
| | Inducible | Inducible | Inducible | Inducible | Inducible | Inducible |
| | PD1ssGluc | IL2FLGluc | IL2FLGluc | Her3(ss + 4)Gluc | TNFaFLGluc | TNFaFLGluc |
| Compound | 293FRT/TO: Mean | 293FRT/TO: Mean | 293FRT/TO: | 293FRT/TO: Mean | 293FRT/TO: Mean | 293FRT/TO: |
| ID | IC50 (nM) | IC50 (nM) | IL2/PD1 | IC50 (nM) | IC50 (nM) | TNF/PD1 |
| A1 | 26.87 | 53.82 | 2 | 58.42 | 368.6 | 13.7 |
| A12 | 18.78 | 37.51 | 2 | | | |
| A13 | 20719.62 | I.A. | >1.2 | | | |
| A15 | 34.17 | 97.11 | 2.8 | 72.49 | 875.36 | 25.6 |
| A16 | 5.06 | 10.63 | 2.1 | 8.37 | 126.77 | 25 |
| A17 | 6.69 | 16.68 | 2.5 | 11.47 | 177.7 | 26.6 |
| A18 | 64.2 | 144.84 | 2.3 | 108.82 | 1020.97 | 15.9 |
| A19 | 2.46 | 9.11 | 3.7 | 5.66 | 45.31 | 18.4 |
| A2 | 18.17 | 34.01 | 1.9 | 31.37 | 211 | 11.6 |
| A20 | 16.44 | 39.59 | 2.4 | 40.33 | 131.44 | 8 |
| A21 | 0.89 | 3.87 | 4.3 | 2.61 | 27.15 | 30.4 |
| A25 | 9.92 | 17.51 | 1.8 | 20.67 | 240.47 | 24.2 |
| A26 | 3.15 | 6.78 | 2.1 | 9.38 | 158.33 | 50.2 |
| A27 | 57.88 | 143.65 | 2.5 | 108.75 | 1104.62 | 19.1 |
| A28 | 5.73 | 10.81 | 1.9 | 11.55 | 136.85 | 23.9 |
| A29 | 534.29 | 2216.14 | 4.1 | 1127.65 | 13050.6 | 24.4 |
| A3 | 15.59 | 26.03 | 1.7 | 33.51 | 171.29 | 11 |
| A4 | 15.99 | 25.66 | 1.6 | 26.84 | 154.91 | 9.69 |
| A5 | 14.7 | 22.7 | 1.5 | 25.83 | 86.99 | 5.92 |
| A7 | 13.84 | 35.59 | 2.6 | 25.19 | 329.66 | 23.8 |
| A8 | 293.47 | 805.78 | 2.7 | 588.12 | 13366.47 | 45.5 |
| A9 | 7.22 | 17.12 | 2.4 | | | |
| A44 | 166.91 | 408.47 | 2.4 | 415.02 | 3882.41 | 23.3 |
| A45 | 16.38 | 55.51 | 3.4 | 34.27 | 219.77 | 13.4 |
| A46 | 3 | 4.11 | 1.4 | 6.51 | 51.95 | 17.3 |
| A47 | 43.64 | 82.35 | 1.9 | 83.54 | 554.02 | 12.7 |
| A48 | 6.13 | 13.52 | 2.2 | 17.39 | 100.7 | 16.4 |
| A49 | 778.34 | 2217.79 | 2.8 | 1639.05 | I.A. | >32.1 |
| A51 | 529.42 | 1038.17 | 2 | 917.25 | 19348.27 | 36.5 |
| A52 | 339.83 | 912.47 | 2.7 | 450.08 | 4049.2 | 11.9 |
| A53 | 23.74 | 54.29 | 2.3 | 31.49 | 301.27 | 12.7 |
| A55 | 19.51 | 58.1 | 3 | 41.44 | 426.35 | 21.8 |
| A56 | 5.8/4.66/4.85 | 10.36/6.77/8.74 | 1.8/1.5/1.8 | 9.54/8.51/7.39 | 110.9/63.25/96.94 | 19.1/13.6/20 |
| A57 | 17.74 | 45.07 | 2.5 | 38.27 | 287.13 | 16.2 |
| A58 | 107.91 | 295.82 | 2.7 | 248.68 | 1877.49 | 17.4 |
| A59 | 2182.06 | 6964.96 | 3.2 | 5062.82 | 22465.04 | 10.3 |
| A60 | 63.82 | 186.35 | 2.9 | 130.89 | 860.91 | 13.5 |
| A61 | 916.83 | 3219.14 | 3.5 | 1574.91 | 11630.09 | 12.7 |
| A62 | 15 | 29.82 | 2 | 35.7 | 251.18 | 16.7 |
| A63 | 80 | 148.68 | 1.9 | 130.25 | 1059.99 | 13.2 |
| A64 | 10.64 | 27.02 | 2.5 | 19.44 | 164.12 | 15.4 |
| A65 | 2.11 | 7.99 | 3.8 | 6.49 | 65.5 | 31 |
| A66 | 27.17 | 69.02 | 2.5 | 59.67 | 583.86 | 21.5 |
| A67 | 23.65 | 54.95 | 2.3 | 65.08 | 861.74 | 36.4 |
| A68 | 2.32 | 6.04 | 2.6 | 4.98 | 44.11 | 19 |
| A69 | 52.04 | 115.92 | 2.2 | 60.44 | 330.24 | 6.35 |
| A70 | 13.16 | 30.04 | 2.3 | 18.28 | 236.29 | 18 |
| A71 | 136.16 | 366.43 | 2.7 | 148.36 | 3701.45 | 27.2 |
| A72 | 141.31 | 316.14 | 2.2 | 197 | 2651.51 | 18.8 |
| A73 | 26.59 | 88.47 | 3.3 | 23.57 | 349.84 | 13.2 |
| A74 | 118.17 | 367.06 | 3.1 | 232.49 | 2800 | 23.7 |
| A75 | 2.5 | 8.11 | 3.2 | 0.6 | 71 | 28.4 |
| A76 | 291.58 | 1508.11 | 5.2 | 829.78 | 6663.78 | 22.9 |
| A77 | 1.84 | 3.22 | 1.8 | 3.62 | 19.06 | 10.4 |
| A78 | 13.19 | 30.11 | 2.3 | 27.68 | 158.71 | 12 |
| A79 | 10.77 | 29.69 | 2.8 | 23.25 | 171.32 | 15.9 |
| A80 | 595.48 | 1840.62 | 3.1 | 1074.74 | 11402.11 | 19.1 |
| A81 | 13.99 | 41.5 | 3 | 23.7 | 143.3 | 10.2 |
| A82 | 78.56 | 248.59 | 3.2 | 210.03 | 653.06 | 8.31 |
| A83 | 82.11 | 231.51 | 2.8 | 248.51 | 1530.06 | 18.6 |
| A84 | 40.68 | 87.01 | 2.1 | 62.63 | 309.7 | 7.61 |
| A85 | 285.02/293.76 | 2111.18/1473.87 | 7.4/5 | 566.35/542.87 | 21637.09/I.A. | 75.9/>85.1 |
| A87 | 635.93 | 2577.23 | 4.1 | 1151.27 | 21518.65 | 33.8 |
| A88 | 951.75 | 3332.3 | 3.5 | 1212.55 | 10955.51 | 11.5 |
| A89 | 558.22 | 2713.93 | 4.9 | 1105.09 | 18392.69 | 32.9 |
| A90 | 41.45 | 82.87 | 2 | 82.77 | 512.28 | 12.4 |
| A91 | 14.77 | 25.03 | 1.7 | 26.5 | 147.9 | 10 |
| A92 | 23.27 | 30.63 | 1.3 | 47.27 | 254.27 | 10.9 |
| A93 | 12.35 | 26.33 | 2.1 | 27.09 | 186.79 | 15.1 |
| A94 | 8.73 | 17.83 | 2 | 14.52 | 167.76 | 19.2 |
| A95 | 8.34 | 18.61 | 2.2 | 14.85 | 132.95 | 15.9 |
| A96 | 1.49 | 4.21 | 2.8 | 4.2 | 40.46 | 27.2 |
| A97 | 18.75 | 32.79 | 1.7 | 22.12 | 161.98 | 8.64 |
| A98 | 0.91 | 2.25 | 2.5 | 2.05 | 20.15 | 22.2 |
| A99 | 126.57 | 348.42 | 2.8 | 283.62 | 5913.75 | 46.7 |
| A100 | 22.8 | 34.24 | 1.5 | 32.05 | 195.42 | 8.57 |
| A101 | 47.9 | 94.29 | 2 | 68.16 | 379.26 | 7.92 |
| A102 | 14.97 | 27.54 | 1.8 | 16.85 | 121.37 | 8.11 |
| A103 | 295.86 | 843.45 | 2.9 | 670.19 | 8635.27 | 29.2 |
| A104 | 153.19 | 434.55 | 2.8 | 355.18 | 4030.93 | 26.3 |
| A105 | 53.58 | 65.77 | 1.2 | 91.68 | 523.61 | 9.81 |
| A106 | 16.33 | 26.17 | 1.6 | 15.59 | 119.59 | 7.32 |
| A107 | 204.88 | 461.79 | 2.3 | 321.57 | 2914.85 | 14.2 |
| A108 | 33.69 | 71.25 | 2.1 | 72.88 | 282.69 | 8.39 |
| A109 | 31 | 90.67 | 2.9 | 39.25 | 302.28 | 9.75 |
| A110 | 23.24 | 79.85 | 3.4 | 36.36 | 310.46 | 13.4 |
| A111 | 7.92 | 16.3 | 2.1 | 13.55 | 94.46 | 11.9 |
| A112 | 3.81 | 8.64 | 2.3 | 5.98 | 39.98 | 10.5 |
| A113 | 9.82 | 17.91 | 1.8 | 22.57 | 136.42 | 13.9 |
| A114 | 36.15 | 54.86 | 1.5 | 87.8 | 550.92 | 15.2 |
| A115 | 41.53 | 122.24 | 2.9 | 89.28 | 586.83 | 14.1 |
| A116 | 2 | 4.93 | 2.5 | 4.14 | 39.5 | 19.7 |
| A117 | 28.17 | 50.96 | 1.8 | 33.41 | 301.4 | 10.7 |
| A118 | 103.27 | 129.08 | 1.2 | 135.2 | 888.16 | 8.6 |
| A119 | 1.76 | 7.35 | 4.2 | 6.31 | 41.34 | 23.5 |
| A120 | 24.52 | 42.62 | 1.7 | 34.5 | 157.94 | 6.44 |
| A121 | 10.93 | 20.67 | 1.9 | 17.49 | 180.09 | 16.5 |
| A122 | 7.88 | 15.96 | 2 | 17.43 | 190.95 | 24.2 |
| A123 | 8.21 | 24.28 | 3 | 18.1 | 142.54 | 17.4 |
| A124 | 4.87 | 10.39 | 2.1 | 9.15 | 75.83 | 15.6 |
| A125 | 22.53 | 65.01 | 2.9 | 28.58 | 321.86 | 14.3 |
| A126 | 59.45 | 72.45 | 1.2 | 5.03 | 414.2 | 6.97 |
| A127 | 5.65 | 10.13 | 1.8 | 10.78 | 107.38 | 19 |
| A128 | 37.87 | 59.6 | 1.6 | 68.14 | 494.78 | 13.1 |
| A129 | 9.58 | 21.18 | 2.2 | 15.46 | 302.51 | 31.6 |
| A130 | 15.84 | 65.77 | 4.2 | 27.81 | 683.34 | 43.1 |
| A131 | 30.93 | 64.74 | 2.1 | 48.1 | 508.07 | 16.4 |
| A132 | 57.33 | 104.55 | 1.8 | 94.99 | 1058.8 | 18.5 |
| A133 | 60.96 | 81.7 | 1.3 | 64.25 | 892.59 | 14.6 |
| A134 | 27.27 | 42.43 | 1.6 | 47.79 | 526.49 | 19.3 |
| A135 | 15.22 | 52.46 | 3.4 | 34.22 | 511.86 | 33.6 |
| A136 | 59.59 | 186.03 | 3.1 | 86.68 | 1007.2 | 16.9 |
| A137 | 32.21 | 61.44 | 1.9 | 51.53 | 565.77 | 17.6 |
| A138 | 124.83 | 172.32 | 1.4 | 166.75 | 1600.69 | 12.8 |
| A139 | 20.31 | 58.3 | 2.9 | 37.08 | 459.93 | 22.6 |
| A140 | 13.91 | 33.74 | 2.4 | 9.88 | 360.98 | 25.9 |
| A141 | 27.54 | 54.03 | 2 | 43.86 | 496.68 | 18 |
| A142 | 323.14 | 963.11 | 3 | 660.44 | 6874.74 | 21.3 |
| A143 | 51.89 | 31.18 | 0.6 | 64.82 | 209.33 | 4.03 |
| A144 | 0.36 | 1.33 | 3.7 | 0.81 | 5.9 | 16.3 |
| A145 | 0.31 | 1.11 | 3.6 | 0.83 | 7.51 | 24 |
| A146 | 0.97 | 2.56 | 2.6 | 2.47 | 20.59 | 21.1 |
| A147 | 71.93 | 109.41 | 1.5 | 106.2 | 316.48 | 4.4 |
| A148 | 45.92 | 87.18 | 1.9 | 72.97 | 579.38 | 12.6 |
| A149 | 68.14 | 152.05 | 2.2 | 126.32 | 1012.3 | 14.9 |
| A150 | 49.18 | 130.17 | 2.6 | 82.1 | 757.57 | 15.4 |
| A151 | 21.51 | 58.97 | 2.7 | 43.1 | 385.52 | 17.9 |
| A152 | 2412.93 | 3284.08 | 1.4 | 2132.45 | I.A. | >10.4 |
| A153 | 37.36 | 90.39 | 2.4 | 64.85 | 876.86 | 23.5 |
| A154 | 64.51 | 134.76 | 2.1 | 126.54 | 643.16 | 9.97 |
| A155 | 79.99 | 189.09 | 2.4 | 86.62 | 1343.62 | 16.8 |
| A156 | 115.24 | 348.88 | 3 | 272.83 | 1560.91 | 13.5 |
| A157 | 42.18 | 52.17 | 1.2 | 49.66 | 406.55 | 9.64 |
| A158 | 90.54 | 130.45 | 1.4 | 127.69 | 1055.58 | 11.7 |
| A159 | 27.89 | 80.6 | 2.9 | 35.79 | 789.85 | 28.3 |
| A160 | 48.14 | 95.95 | 2 | 82.44 | 690.73 | 14.3 |
| A161 | 284.22 | 640.01 | 2.3 | 405.95 | 4001.1 | 14.1 |
| A162 | 757.94 | 1769.89 | 2.4 | 1266.98 | I.A. | >33.0 |
| A163 | 16.15 | 39.58 | 2.5 | 29.93 | 328.01 | 20.3 |
| A164 | 7.95 | 19.11 | 2.4 | 17.3 | 148.33 | 18.7 |
| A165 | 29.81 | 45.23 | 1.5 | 53.02 | 386.09 | 13 |
| A166 | 52.81 | 108.69 | 2.1 | 93.72 | 744.18 | 14.1 |
| A167 | 361.56 | 1452.89 | 4 | 602.94 | I.A. | >69.1 |
| A168 | 34.97 | 53.66 | 1.5 | 60.54 | 569.26 | 16.3 |
| A169 | 967 | 2170.76 | 2.2 | 1644.62 | 6896.72 | 7.13 |
| A170 | 68.27 | 83.4 | 1.2 | 81.1 | 686.52 | 10.1 |
| A171 | 3.57 | 6.54 | 1.8 | 4.33 | 71.92 | 20.1 |
| A172 | 2.66 | 5.99 | 2.3 | 5.18 | 50.44 | 19 |
| A173 | 15.93 | 40.38 | 2.5 | 26.9 | 322.61 | 20.3 |
| A174 | 2.62 | 8.01 | 3.1 | 6.19 | 81.1 | 30.9 |
| A175 | 3.02 | 12.89 | 4.3 | 7.95 | 80.67 | 26.7 |
| A176 | 15.04 | 41.78 | 2.8 | 30.98 | 486.77 | 32.4 |
| A177 | 0.93 | 2.79 | 3 | 2.39 | 25.76 | 27.6 |
| A178 | 23.88 | 106.95 | 4.5 | 76.31 | 931.27 | 39 |
| A179 | 2.63 | 9.01 | 3.4 | 6.96 | 137.61 | 52.3 |
| A180 | 4.95 | 14.27 | 52.35 | 3.7 | 49.76 | 440.37 |
| B1 | 128.58 | 19932.06 | 160 | 12979.88 | I.A. | >194 |
| B10 | 19985.43 | I.A. | 1.3 | I.A. | I.A. | >1.25 |
| B11 | 5719.46 | I.A. | 4.4 | I.A. | I.A. | >4.37 |
| B12 | I.A. | I.A. | 1 | I.A. | I.A. | <=>1.00 |
| B13 | I.A. | I.A. | 1 | I.A. | I.A. | <=>1.00 |
| B16 | 3985.3 | I.A. | >6.3 | | | |
| B18 | 88.25 | 1564.71 | 18 | 1100.95 | 4700.19 | 53.3 |
| B2 | 220.16 | I.A. | 110 | I.A. | I.A. | >114 |
| B20 | 5417.64 | I.A. | >4.6 | | | |
| B3 | 466.44 | I.A. | 54 | I.A. | I.A. | >53.6 |
| B4 | 504.33 | I.A. | 50 | I.A. | I.A. | >49.6 |
| B5 | 2160.61 | I.A. | 12 | I.A. | I.A. | >11.6 |
| B6 | 4402.66 | I.A. | 5.7 | I.A. | I.A. | >5.68 |
| B7 | 329.61 | 1266.63 | 3.8 | 800.3 | 7871.72 | 23.9 |
| B8 | 260.04 | 874.99 | 3.4 | 609.55 | 3532.77 | 13.6 |
| B9 | 909.9 | 1852.54 | 2 | 1613.05 | I.A. | >27.5 |
| B33 | 63.71 | 9186.34 | 140 | 5561.78 | I.A. | >392 |
| B34 | 778.34 | I.A. | >32 | I.A. | I.A. | >32.1 |
| B35 | 465.85 | I.A. | >54 | I.A. | I.A. | >53.7 |
| B36 | 173.08 | 2506.79 | 14 | 772.68 | 3748.27 | 21.7 |
| B37 | 68.32 | I.A. | >370 | I.A. | I.A. | >366 |
| B38 | 192.45 | 5586.97 | 29 | 1382.48 | 7053.06 | 36.6 |
| B39 | 267.8 | 1957.13 | 7.3 | 944.64 | 6461.27 | 24.1 |
| B40 | 220.77 | 4567.8 | 21 | 1676.38 | I.A. | >113 |
| B41 | 398.24 | >16582.66 | >42 | 3630.61 | I.A. | >62.8 |
| B42 | 6678.16 | I.A. | >3.7 | I.A. | I.A. | >3.74 |
| B43 | 264.49 | I.A. | >95 | I.A. | I.A. | >94.5 |
| B44 | 3361.53 | I.A. | >7.4 | I.A. | I.A. | >7.44 |
| B45 | 42.15 | >22559.02 | >540 | I.A. | I.A. | >593 |
| B47 | 41.46 | 14525.55 | 350 | 5750.96 | 18816.52 | 454 |
| B48 | 22.54 | 576.22 | 26 | 127.19 | 4135.65 | 184 |
| B49 | 27.45 | 1255.33 | 46 | 130.82 | 2509.35 | 91.4 |
| B50 | 339.14 | I.A. | >74 | 11410.21 | I.A. | >73.7 |
| B51 | 5377.28 | I.A. | >4.6 | I.A. | I.A. | >4.65 |
| B52 | 25.51 | 2102.65 | 82 | 734.91 | 4844.08 | 190 |
| B53 | 38.11 | 1673.7 | 44 | 878.11 | 3843.29 | 101 |
| B54 | 6.85 | 5523.48 | 810 | 2954.01 | 9416.82 | 1370 |
| B55 | 25.27 | 2008.74 | 79 | 1169.94 | 6616.76 | 262 |
| B56 | 520.39 | I.A. | >48 | I.A. | I.A. | >48.0 |
| B57 | 2863.67 | I.A. | >8.7 | I.A. | 16687.44 | 5.83 |
| B58 | 24.38 | >19663.51 | >810 | 12417.45 | 2306.88 | 952 |
| B59 | 466.83 | 6094.83 | 13 | 2104.81 | 13852.53 | 29.7 |
| B60 | 139.73 | 1750.94 | 13 | 724.85 | I.A. | >179 |
| B61 | I.A. | I.A. | <=>1.0 | I.A. | I.A. | <=>1.00 |
| B62 | 321.05 | I.A. | >78 | I.A. | I.A. | >77.9 |
| B63 | >20866.55 | I.A. | <=>1.2 | I.A. | I.A. | <=>1.20 |
| B64 | 20.57 | 1723.25 | 84 | 784.38 | 18385.58 | 894 |
| B65 | 120.45 | I.A. | >210 | I.A. | I.A. | >208 |
| B66 | 15518.57 | I.A. | >1.6 | I.A. | I.A. | >1.61 |
| B67 | 5366.94 | 19061.88 | 3.6 | 18455.53 | I.A. | >4.66 |
| B68 | 347.53 | I.A. | >72 | 18172.58 | I.A. | >71.9 |
| B69 | 119.4 | I.A. | >210 | I.A. | I.A. | >209 |
| B70 | 498.99 | I.A. | >50 | I.A. | I.A. | >50.1 |
| B71 | 298.04 | I.A. | >84 | I.A. | I.A. | >83.9 |
| B72 | 7147.12 | I.A. | >3.5 | 10634.12 | I.A. | >3.50 |
| B73 | 486.35 | I.A. | >51 | I.A. | I.A. | >51.4 |
| B74 | 417.86 | 5358.13 | 13 | 4075.22 | 9975 | 23.9 |
| B75 | 445.82 | 17086.56 | 38 | 5665.36 | 10271.07 | 23 |
| B76 | 419.31 | I.A. | >60 | 1597.01 | I.A. | >59.6 |
| B77 | 490.72 | I.A. | >51 | 11477.73 | I.A. | >50.9 |
| B78 | 418.1 | I.A. | >60 | I.A. | I.A. | >59.8 |
| B79 | 416.29 | 4343.78 | 10 | 3576.11 | I.A. | >60.1 |
| B80 | 372.29 | 5374.41 | 14 | 3141.02 | 7273.2 | 19.5 |
| B81 | 228.34 | I.A. | >110 | 15611.51 | I.A. | >109 |
| B83 | 319.8 | 3229.01 | 10 | 1962.26 | 11716.77 | 36.6 |
| B84 | 904.67 | 2174.71 | 2.4 | 2020.26 | 1665.32 | 1.84 |
| B85 | 329.34 | I.A. | >76 | I.A. | I.A. | >75.9 |
| B86 | 104.4 | I.A. | >240 | 23203.36 | I.A. | >239 |
| B87 | 132.23 | I.A. | >190 | I.A. | I.A. | >189 |
| B88 | 624.65 | I.A. | >40 | I.A. | I.A. | >40.0 |
| B89 | 271.06 | 1650 | 6.1 | 1101.58 | I.A. | >92.2 |
| B90 | 167.04 | 1170.19 | 7 | 689.58 | I.A. | >150 |
| B91 | 6053.51 | I.A. | >4.1 | I.A. | I.A. | >4.13 |
| B92 | 72.37 | 12107.87 | 170 | I.A. | I.A. | >345 |
| B93 | 686.01 | 10959.27 | 16 | 2730.64 | I.A. | >36.4 |
| B94 | 1021.23 | I.A. | >24 | 876.86 | I.A. | >24.5 |
| B95 | 781.75 | I.A. | >32 | I.A. | I.A. | >32.0 |
| B96 | 303.79 | 7589.84 | 25 | 3295.84 | I.A. | >82.3 |
| B97 | 243.84 | 7510.84 | 31 | 4792.32 | I.A. | >103 |
| B98 | 30.72 | 2285.91 | 74 | 1585.2 | I.A. | >814 |
| B99 | 879.89 | I.A. | >28 | 10117.23 | I.A. | >28.4 |
| B100 | 177.87 | I.A. | >140 | I.A. | I.A. | >141 |
| B101 | 11.64 | 6525.08 | 560 | 4278.82 | 19036.46 | 1640 |
| B102 | 36.7 | 6357.34 | 170 | 4556.27 | I.A. | >681 |
| B103 | 10058.04 | I.A. | >2.5 | I.A. | I.A. | >2.49 |
| B104 | 6.16 | 1281.43 | 210 | 785.4 | 4986.48 | 809 |
| B105 | 23.84 | 2842.31 | 120 | 2240.54 | I.A. | >1050 |
| B106 | 691.94 | 3006.7 | 4.3 | 1279.59 | I.A. | >36.1 |
| B107 | 476.21 | I.A. | >52 | I.A. | I.A. | >52.5 |
| B108 | 6035.22 | I.A. | >4.1 | I.A. | I.A. | >4.14 |
| B109 | I.A. | I.A. | <=>1.0 | I.A. | I.A. | <=>1.00 |
| B110 | 2209.22 | I.A. | >11 | I.A. | I.A. | >11.3 |
| B111 | I.A. | I.A. | <=>1.0 | I.A. | I.A. | ⇔1.00 |
| B112 | 217.42 | I.A. | >110 | I.A. | I.A. | >115 |
| B113 | I.A. | I.A. | <=>1.0 | I.A. | I.A. | <=>1.00 |
| B114 | 5256.05 | I.A. | >4.8 | I.A. | I.A. | >4.76 |
| B115 | I.A. | I.A. | <=>1.0 | I.A. | I.A. | <=>1.00 |
| B116 | 10.53 | 5269.6 | 500 | 3693.25 | 18792.53 | 1780 |
| B117 | 31.76 | I.A. | >790 | I.A. | 23606.25 | 743 |
| B118 | 247.11 | I.A. | >100 | I.A. | I.A. | >101 |
| B119 | 51.09 | 10096.52 | 200 | 8903.07 | 21338.41 | 418 |
| B120 | 2692.49 | I.A. | >9.3 | I.A. | I.A. | >9.29 |
| C1 | 455.19 | 1258.38 | 2.8 | 1329.94 | 10048.83 | 22.1 |
| C10 | 5207.34 | 7086.31 | 1.4 | 13895.75 | I.A. | >4.80 |
| C11 | I.A. | I.A. | 1 | I.A. | I.A. | <=>1.00 |
| C12 | I.A. | I.A. | 1 | I.A. | I.A. | <=>1.00 |
| C13 | I.A. | I.A. | 1 | I.A. | I.A. | <=>1.00 |
| C14 | I.A. | I.A. | 1 | I.A. | I.A. | <=>1.00 |
| C15 | I.A. | I.A. | 1 | I.A. | I.A. | <=>1.00 |
| C16 | 8078.07 | 6904.17 | 0.85 | 8931.4 | I.A. | >3.09 |
| C17 | 22266.51 | 6747.09 | 0.3 | I.A. | I.A. | >1.12 |
| C2 | 482.54 | 1515.61 | 3.1 | 1377.45 | 9737.83 | 20.2 |
| C21 | I.A. | I.A. | <=>1.0 | | | |
| C23 | 5087.73 | 7649.13 | 1.5 | 17534.7 | I.A. | >4.91 |
| C26 | 7973.87 | I.A. | >3.1 | | | |
| C27 | 670.53 | 2632.57 | 3.9 | | | |
| C29 | 4886.34 | I.A. | >5.1 | | | |
| C3 | 1114.02 | 3036.67 | 2.7 | 2267.37 | 15069.54 | 13.5 |
| C30 | 5205.16 | 5622.5 | 1.1 | | | |
| C31 | 4123.66 | 7047.12 | 1.7 | | | |
| C32 | 5378.08 | 7812.39 | 1.5 | 8808.06 | I.A. | >4.65 |
| C33 | 9672.83 | 6050.49 | 0.63 | | | |
| C37 | I.A. | I.A. | <=>1.0 | | | |
| C39 | 2746.67 | 3354.13 | 1.2 | | | |
| C4 | 782.37 | 1937.43 | 2.5 | 1542.94 | 16489.19 | 21.1 |
| C49 | 909.39 | 1636.8 | 1.8 | | | |
| C5 | 2200.51 | 2878.12 | 1.3 | 2415.73 | I.A. | >11.4 |
| C50 | 3138.44 | 2057.62 | 0.66 | | | |
| C53 | 14.65 | 71.88 | 4.9 | 38.67 | 266.44 | 18.2 |
| C54 | 132.32 | 478.89 | 3.6 | 272.05 | 1792.1 | 13.5 |
| C55 | 167.89 | 739.81 | 4.4 | 373.72 | 2564.92 | 15.3 |
| C56 | 1747.65 | 4666.01 | 2.7 | 3271.12 | 19154.56 | 11 |
| C57 | 357.22 | 1072.14 | 3 | | | |
| C58 | 7876.18 | I.A. | 3.2 | 9104.52 | I.A. | >3.17 |
| C59 | 7975.15 | I.A. | 3.1 | 12225.21 | I.A. | >3.13 |
| C6 | 2603.75 | 7292.13 | 2.8 | 5224.65 | I.A. | >9.60 |
| C60 | 8593.84 | I.A. | 2.9 | I.A. | I.A. | >2.91 |
| C61 | 8964.69 | 16683.54 | 1.9 | 20418.03 | I.A. | >2.79 |
| C62 | 8439.43 | I.A. | >3.0 | | | |
| C7 | 3701.51 | I.A. | 6.8 | 7972.26 | I.A. | >6.75 |
| C8 | 3784.81 | 22379.24 | 5.9 | 9474.06 | I.A. | >6.61 |
| C9 | 4556.9 | 7539.86 | 1.7 | 6098.14 | I.A. | >5.49 |
| C84 | 4248.7 | I.A. | >5.9 | 6594.27 | I.A. | >5.88 |
| C85 | 126.1 | 279.4 | 2.2 | 257.29 | 1201.73 | 9.53 |
| C86 | 2983 | 2376.09 | 0.8 | 1105.19 | I.A. | >8.38 |
| C87 | 78.44 | 238.37 | 3 | 129.37 | 731.16 | 9.32 |
| C88 | 580.57 | 1895.55 | 3.3 | 942.12 | 5932.89 | 10.2 |
| C89 | 1182.79 | 2863.23 | 2.4 | 1792.34 | 14541.25 | 12.3 |
| C90 | 1649.68 | 4014.53 | 2.4 | 2389.45 | 16102.08 | 9.76 |
| C91 | 93.37 | 344.7 | 3.7 | 127.02 | 1244.4 | 13.3 |
| C92 | 2210.32 | 5548.42 | 2.5 | 3011.13 | 24529.48 | 11.1 |
| C93 | 4266.98 | 1625.52 | 0.38 | 1075.87 | I.A. | >5.86 |
| C94 | 3585.25 | 3661.56 | 1 | 2403.93 | I.A. | >6.97 |
| C95 | 5.05 | 15.52 | 3.1 | 3.5 | 103.57 | 20.5 |
| C96 | 63.74 | 280.34 | 4.4 | 59.58 | 942.72 | 14.8 |
| C97 | 2995.99/4652.27 | 6146.89/>16941.91 | 2.1/>3.6 | 3226.26/19582.55 | I.A. | >8.34/>5.37 |
| C99 | 1723.68 | 5384.42 | 3.1 | 2827.4 | 16758.05 | 9.72 |
| C100 | 7638.17 | 6455.72 | 0.85 | 15938.49 | I.A. | >3.27 |
| C102 | 44.24 | 97.47 | 2.2 | 108.72 | 983.7 | 22.2 |
| C103 | 665 | 1155.59 | 1.7 | 1336.77 | 4985.61 | 7.5 |
| C104 | 142.8 | 412.5 | 2.9 | 350.01 | 2405.91 | 16.8 |
| C105 | 36.01 | 157.39 | 4.4 | 107.07 | 899.79 | 25 |
| C106 | 1092.87 | 2638.86 | 2.4 | 1432.68 | 19395.79 | 17.7 |
| C107 | 32.2 | 87.11 | 2.7 | 56.16 | 792.78 | 24.6 |
| C108 | 2402.44/1589.86 | 4513.47/3475.32 | 1.9/2.2 | 2414.92/2482.07 | I.A./17898.66 | >10.4/11.3 |
| C110 | 31.4 | 94.94 | 3 | 46.41 | 301.93 | 9.61 |
| C111 | 317.91 | 653.89 | 2.1 | 294.91 | 4938.16 | 15.5 |
| C112 | 545.2 | 2289.45 | 4.2 | 2236.58 | 9737.2 | 17.9 |
| C113 | 879.05 | 1795.28 | 2 | 1326.84 | 16314.67 | 18.6 |
| C114 | 6900.35 | I.A. | >3.6 | I.A. | I.A. | >3.62 |
| C115 | 286.7 | 613.46 | 2.1 | 551.5 | 3530.62 | 12.3 |
| C116 | 1032.18 | 1590.01 | 1.5 | 1058.74 | 7537.12 | 7.3 |
| C117 | 25.64 | 54.28 | 2.1 | 49.59 | 274.87 | 10.7 |
| C118 | 719.23 | 1008.39 | 1.4 | 732.05 | 9784.01 | 13.6 |
| C119 | 2123.63 | 1624.33 | 0.76 | 1507.66 | I.A. | >11.8 |
| C120 | 4415.8 | 2061.18 | 0.47 | 1830.53 | I.A. | >5.66 |
| C121 | 2307.81 | 1334.94 | 0.58 | 1205.67 | I.A. | >10.8 |
| C122 | 1075.22 | 1350.7 | 1.3 | 1004.8 | I.A. | >23.3 |
| C123 | 377 | 775.6 | 2.1 | 620.42 | 8552.87 | 22.7 |
| D2 | 396.85 | >13529.39 | >34 | 1497.76 | 6085.83 | 15.3 |
| D3 | 733.68 | 6663.29 | 9.1 | 1999.13 | I.A. | >34.1 |
| D4 | 1035.89 | I.A. | >24 | 10603.72 | I.A. | >24.1 |
| D5 | 776.33 | 13450.52 | 17 | 21391.33 | I.A. | >32.2 |
| D6 | 595.19 | I.A. | >42 | 6198.2 | I.A. | >42.0 |
| D7 | 2826.96 | I.A. | >8.8 | I.A. | I.A. | >8.84 |
| E1 | 153.7 | 22074.01 | 140 | 4127.73 | I.A. | >163 |
| E10 | 5762.05 | 5408.06 | 0.94 | 4925.38 | I.A. | >4.34 |
| E11 | 2184.07 | 8242.06 | 3.8 | 8166.28 | I.A. | >11.4 |
| E12 | 124.49 | 431.97 | 3.5 | 213.36 | 7891.28 | 63.4 |
| E13 | 53.32 | 183.82 | 3.4 | 113.51 | 831.98 | 15.6 |
| E14 | 4237.05 | 6615.74 | 1.6 | 20715.63 | I.A. | >5.90 |
| E15 | I.A. | I.A. | 1 | I.A. | I.A. | <=>1.00 |
| E16 | I.A. | I.A. | 1 | I.A. | I.A. | <=>1.00 |
| E17 | I.A. | I.A. | 1 | I.A. | I.A. | <=>1.00 |
| E18 | >5000.00 | >5000.00 | 1 | >5000.00 | >5000.00 | <=>1.00 |
| E2 | 23.36 | 529.77 | 23 | 408.98 | 4849.47 | 208 |
| E20 | 2520.4 | 4996.61 | 2 | | | |
| E21 | 6481.26 | I.A. | >3.9 | I.A. | I.A. | >3.86 |
| E22 | 129.51 | 398.58 | 3.1 | 216.47 | 3424.32 | 26.4 |
| E23 | 337.51 | 772.71 | 2.3 | 629.2 | 11419.89 | 33.8 |
| E26 | 9.53 | 22.9 | 2.4 | | | |
| E27 | 17.48 | 56.76 | 3.2 | | | |
| E3 | 797.97 | 13052.45 | 16 | 7179.43 | I.A. | >31.3 |
| E30 | I.A. | I.A. | 1 | I.A. | I.A. | <=>1.00 |
| E31 | 1665.02 | 9022.21 | 5.4 | 9144.53 | I.A. | >15.0 |
| E32 | 1024 | 3942.44 | 3.9 | 3057.2 | 7079.06 | 6.91 |
| E33 | 1230.34 | 14787.36 | 12 | 3922.27 | I.A. | >20.3 |
| E34 | 312.25 | 2603.37 | 8.3 | 648.31 | 8140.22 | 26.1 |
| E35 | 148.08 | 927.43 | 6.3 | 387.71 | 4832.93 | 32.6 |
| E36 | 284.03 | 1707.61 | 6 | 889.98 | 5068.59 | 17.8 |
| E37 | 2895.76 | 16540.77 | 5.7 | 8040.95 | I.A. | >8.63 |
| E38 | 493.27 | 1868.03 | 3.8 | 1118.94 | 5276.01 | 10.7 |
| E39 | 159.07 | 448.63 | 2.8 | 343.53 | 1646.13 | 10.3 |
| E4 | 438.64 | 5201.59 | 12 | 5082.14 | I.A. | >57.0 |
| E40 | 320.3 | 770.58 | 2.4 | 644.79 | 2054.78 | 6.42 |
| E41 | 300.62 | 537.73 | 1.8 | 569.95 | 1562.95 | 5.2 |
| E42 | 472.6 | 2586.53 | 5.5 | 1122.69 | 8114.13 | 17.2 |
| E43 | 1135.37 | 10894.87 | 9.6 | 3154.88 | 21377.96 | 18.8 |
| E44 | 452.72 | 2722.08 | 6 | 1319.79 | 11552.16 | 25.5 |
| E45 | 531.32 | 2365.3 | 4.5 | 1447.76 | 6032.26 | 11.4 |
| E46 | 29.13 | 114.6 | 3.9 | 60.4 | 528.6 | 18.1 |
| E47 | 547.4 | 1435.14 | 2.6 | 1429.1 | 4064.69 | 7.43 |
| E48 | 7893.71 | 7833.18 | 0.99 | 13513.98 | I.A. | >3.17 |
| E5 | 125.34 | 1234.56 | 9.8 | 1055.6 | I.A. | >199 |
| E50 | 1957.6 | 5558.04 | 2.8 | 4374.58 | I.A. | >12.8 |
| E51 | I.A. | I.A. | 1 | I.A. | I.A. | <=>1.00 |
| E52 | I.A. | I.A. | 1 | I.A. | I.A. | <=>1.00 |
| E53 | I.A. | I.A. | 1 | I.A. | I.A. | <=>1.00 |
| E55 | 315.38 | 2983.82 | 9.5 | | | |
| E56 | 91.64 | 4355.15 | 48 | | | |
| E57 | 9.67 | 2163.88 | 220 | | | |
| E58 | 8240.85 | I.A. | >3.0 | | | |
| E59 | 407.94 | 932.16 | 2.3 | 580.85 | 12618.83 | 30.9 |
| E6 | 207.59 | 1780.29 | 8.6 | 1280.02 | 6041.48 | 29.1 |
| E60 | 802.45 | 1692.15 | 2.1 | 1280.87 | I.A. | >31.2 |
| E7 | 158.46 | 1191.4 | 7.5 | 946.7 | 2755.96 | 17.4 |
| E8 | 695.49 | 5047.59 | 7.3 | 4956.25 | I.A. | >35.9 |
| E9 | 774.69 | 3638.95 | 4.7 | 3917.3 | 19460.59 | 25.1 |
| E62 | 2.94 | 9.89 | 3.4 | 8.52 | 42.96 | 14.6 |
| E63 | 64.03 | 95.18 | 1.5 | 97.04 | 703.78 | 11 |
| E64 | 70.33 | 70.52 | 1 | 106.14 | 377.34 | 5.37 |
| E65 | 5.5 | 16.41 | 3 | 14.37 | 70.97 | 12.9 |

I.A. indicates IC50 > 25000

TABLE 2

| | | | Liver Microsome | Liver Microsome | Liver Microsome | Liver Microsome |
|---|---|---|---|---|---|---|
| | 48 hr H929 | 48 hr U266 | Stability (Multiple | Stability (Multiple | Stability (Multiple | Stability (Multiple |
| | Viability | Viability | Species): Mouse - | Species): Rat - | Species): Monkey - | Species): Human - |
| | Celltiter- | Celltiter- | % Remaining after | % Remaining after | % Remaining after | % Remaining after |
| Compound | Glo: Mean EC50 | Glo: Mean EC50 | 30 min w/NADPH | 30 min w/NADPH | 30 min w/NADPH | 30 min w/NADPH |
| ID | (nM) | (nM) | (%) | (%) | (%) | (%) |
| A1 | 1232.85 | I.A. | 24.8 | 50 | 6.47 | 14.3 |
| A12 | 1046.78 | I.A. | 3.86 | 0.58 | 0.23 | 0.31 |
| A13 | I.A. | I.A. | 6.8 | 5.37 | 0.39 | 1.91 |
| A15 | 767.02 | 7345.76 | 10.5 | 17.7 | 1.86 | 5.24 |
| A16 | 119.32 | 2144.04 | 0.26 | 0.15 | 0.17 | 0.34 |
| A17 | 160.75 | I.A. | 2.94 | 0.38 | 0.22 | 0.32 |
| A18 | 1584.81 | I.A. | 3.75 | 0.17 | 0.08 | 0.05 |
| A19 | 219.31 | I.A. | 3.24 | 0.39 | 0.17 | 0.48 |
| A2 | 1342.06 | I.A. | 6.6 | 7.5 | 0.18 | 0.92 |
| A20 | 339.84 | 4700.99 | 7.28 | 26.3 | 2.72 | 4.63 |
| A21 | 54.12 | I.A. | 6.31 | 11 | 1.43 | 4.28 |
| A22 | | | 15.8 | 11.7 | 17.6 | 29.1 |
| A23 | | | 25.3 | 40.2 | 29.9 | 24.7 |
| A24 | | | 19.9 | 35.9 | 22.5 | 22.4 |
| A25 | 412.01 | 16138.99 | 1.56 | 1.84 | 1.66 | 3.01 |
| A26 | 135.88 | 1166.83 | 5.75 | 8.78 | 3.34 | 5.11 |
| A27 | 5792.2 | I.A. | 6.54 | 0.33 | 0.51 | 1.61 |
| A28 | 251 | 10052.95 | 30.3 | 51.5 | 3.97 | 16.4 |
| A29 | I.A. | I.A. | 1.4 | 4.6 | 0.39 | 0.59 |
| A3 | 530.33 | I.A. | 9 | 20.1 | 0.62 | 2.33 |
| A30 | | | 0.86 | 0.21 | 0.03 | 0.27 |
| A32 | | | 0.17 | 0.55 | 0.05 | 0.24 |
| A4 | 430.32 | I.A. | 9.36 | 15.4 | 0.36 | 4.21 |
| A5 | 448.14 | I.A. | 13 | 26.5 | 4.26 | 10.4 |
| A6 | | | 37.4 | 44.8 | 25.4 | 25.7 |
| A7 | 497 | I.A. | 39.9 | 5.67 | 3.56 | 4.64 |
| A8 | 2000.77 | 2935.17 | 3.14 | 11 | 11.3 | 3.14 |
| A9 | 519.24 | I.A. | 19.8 | 0.8 | 0.23 | 0.71 |
| A44 | 3312.25 | 6949.52 | 62.6 | 30 | 26 | 28.6 |
| A45 | 424.65 | I.A. | 0.76 | 0.29 | 0.1 | 0.08 |
| A46 | 79.05 | I.A. | 4.16 | 9.73 | 2.39 | 3.12 |
| A47 | 1205.48 | I.A. | 0.02 | 1.17 | 0.06 | 1.82 |
| A48 | 149.58 | I.A. | 4.71 | 0.64 | 1.44 | 2.91 |
| A49 | 9862.06 | 8116.39 | 29.4 | 10.7 | 0.94 | 1.94 |
| A51 | 4951.1 | 6063.02 | 64.3 | 0.4 | 1.91 | 2.67 |
| A52 | 20561.51 | I.A. | 3.47 | 0.63 | 0.33 | 1.11 |
| A53 | 20561.51 | I.A. | 3.47 | 0.63 | 0.33 | 1.11 |
| A55 | 681.6 | I.A. | 18.3 | 0.11 | 0.1 | 0.34 |
| A56 | 152.05/149.15/165.13 | I.A./>18783.99/ | 81.1/81.8 | 48.3/54.8 | 27.3/25.8 | 43.9/46 |
| | | >24804.19 | | | | |
| A57 | 499.6 | I.A. | 6.1 | 6.28 | 49.5 | 26.6 |
| A58 | 3342.86 | 9988.7 | 0.24 | 4.13 | 0.29 | 0.4 |
| A59 | 15993.82 | 6493.33 | 49.8 | 21.3 | 2.29 | 0.62 |
| A60 | 4717.42 | I.A. | 48.3 | 19.7 | 23.8 | 13.7 |
| A61 | 6287.3 | 5519.18 | 54.5 | 22.8 | 5.11 | 2.45 |
| A62 | 373.32 | I.A. | 52.7 | 45.1 | 21.4 | 34.3 |
| A63 | 1778.2 | I.A. | 3.64 | 6.07 | 1.18 | 2.22 |
| A64 | 202.65 | I.A. | 13.7 | 6.39 | 0.78 | 1.39 |
| A65 | 81.2 | I.A. | 7.68 | 1.1 | 0.35 | 22.1 |
| A66 | 416.28 | I.A. | 30 | 5.19 | 2.72 | 2.98 |
| A67 | 561.6 | 6864.9 | 82.3 | 51.9 | 40.4 | 28.9 |
| A68 | 44.61 | I.A. | 27.5 | 16.9 | 11.8 | 25.8 |
| A69 | 3179.27 | I.A. | 12.1 | 35 | 0.47 | 1.43 |
| A70 | 385.05 | I.A. | 23.7 | 7.79 | 0.83 | 2.86 |
| A71 | 4407.05 | 7802.46 | 95.6 | 82.1 | 36.1 | 51.8 |
| A72 | 3771.84 | 6553.36 | 72.3 | 94.1 | 37.8 | 23.9 |
| A73 | 3750.45 | I.A. | 0.14 | 15 | 0.08 | 0.1 |
| A74 | 3866.1 | 6125.32 | 94.3 | 98.3 | 38.5 | 38.6 |
| A75 | 251.27 | I.A. | 3.74 | 0.05 | 0.87 | 6.4 |
| A76 | 22391.59 | I.A. | 11 | 4.99 | 1.43 | 2.44 |
| A77 | 60.1 | I.A. | 79.6 | 38.1 | 22.4 | 72.8 |
| A78 | 494.69 | I.A. | 6.09 | 3.42 | 3.13 | 6.01 |
| A79 | 345.43 | I.A. | 5.88 | 37.7 | 0.78 | 5.48 |
| A80 | 5729.56 | 7783.21 | 71.9 | 88.1 | 17.6 | 51 |
| A81 | 678.6 | I.A. | 39.5 | 68.9 | 8.77 | 12.8 |
| A82 | 5660.96 | I.A. | 0.84 | 15.3 | 0.16 | 3.95 |
| A83 | 4001.4 | I.A. | 0.55 | 16.6 | 0.16 | 0.64 |
| A84 | 1049.86 | I.A. | 1 | 2.51 | 0.73 | 0.5 |
| A85 | 6258.12/5294.32 | 10257.52/7378.5 | 57.7/62.5 | 90.7/99.6 | 49/62.5 | 52.8/65.2 |
| A87 | 6314.71 | 8827.89 | 75.6 | 61.8 | 32.6 | 51.1 |
| A88 | 7328.52 | 11490.92 | 71.7 | 76 | 3.2 | 57.6 |
| A89 | 6365.12 | 8303.23 | 40.4 | 56.9 | 2.45 | 47 |
| A90 | 1079.56 | I.A. | 21.7 | 39.1 | 1.3 | 22.3 |
| A91 | 346.47 | I.A. | 27.4 | 19.7 | 2.34 | 43 |
| A92 | 462.61 | I.A. | 52.3 | 43.6 | 22.5 | 63.5 |
| A93 | 389.7 | I.A. | 1.18 | 0.11 | 0.14 | 4.24 |
| A94 | 642 | I.A. | 10.9 | 12.8 | 0.9 | 11.1 |
| A95 | 589.92 | I.A. | 10.2 | 13.9 | 0.88 | 8.25 |
| A96 | 34.2 | I.A. | 0.28 | 1 | 0.12 | 1.42 |
| A97 | 447.81 | I.A. | 0.22 | 42.4 | 0.4 | 6.01 |
| A98 | 22.43 | I.A. | 1.29 | 1.18 | 0.48 | 2.43 |
| A99 | 5301.53 | I.A. | 1.28 | 0.49 | 0.12 | 1.85 |
| A100 | 442.5 | I.A. | 3.12 | 14.8 | 0.24 | 3.16 |
| A101 | 1270.02 | 12826.13 | 62.7 | 76.5 | 14.5 | 2.2 |
| A102 | 316.8 | I.A. | 7.68 | 19.7 | 1.22 | 8.26 |
| A103 | 4040.17 | 5655.13 | 55 | 28.3 | 47.2 | 67 |
| A104 | 3170.46 | 5764.46 | 16.5 | 27.9 | 51.6 | 68.8 |
| A105 | 1030.46 | I.A. | 1.29 | 16.9 | 0.98 | 0.87 |
| A106 | 472.29 | I.A. | 1.43 | 3.12 | 0.35 | 2.95 |
| A107 | 4545.12 | 7281.14 | 6 | 64.6 | 12.7 | 23.1 |
| A108 | 1276.82 | I.A. | 17 | 39 | 0.49 | 13.2 |
| A109 | 1234.4 | I.A. | 21 | 55.6 | 2.02 | 13.8 |
| A110 | 883.79 | I.A. | 31.2 | 55.7 | 4.58 | 10.5 |
| A111 | 422.47 | I.A. | 12.7 | 7.97 | 3.48 | 2.78 |
| A112 | 407.72 | I.A. | 5.81 | 6.03 | 0.35 | 3.41 |
| A113 | 615.1 | I.A. | 4.74 | 0.64 | 1.3 | 0.61 |
| A114 | 1023.71 | I.A. | 13 | 8.6 | 0.43 | 3.96 |
| A115 | 1997.02 | 7269.64 | 3.39 | 9.02 | 3.2 | 14.6 |
| A116 | 160.33 | I.A. | 60.7 | 37.7 | 18.9 | 45.3 |
| A117 | 937.97 | I.A. | 0.81 | 8.86 | 0.09 | 2.64 |
| A118 | 1798.34 | I.A. | | | | |
| A119 | 132.98 | I.A. | | | | |
| A120 | 830.96 | I.A. | | | | |
| A121 | 446.85 | I.A. | 7.09 | 9.82 | 1.57 | 8.26 |
| A122 | 354.38 | 8019.04 | 17.1 | 18.8 | 27.3 | 35.8 |
| A123 | 289.41 | I.A. | 23.3 | 44.7 | 31.6 | 49.3 |
| A124 | 165.74 | I.A. | 31.2 | 37.6 | 27.8 | 53.1 |
| A125 | 1214.1 | I.A. | 45.1 | 24.9 | 10.1 | 26.2 |
| A126 | 1125.62 | I.A. | 17.7 | 0.78 | 0.92 | 1.21 |
| A127 | 277.57 | I.A. | 3.95 | 0.26 | 24.4 | 30.6 |
| A128 | 847.34 | I.A. | 25.5 | 31.3 | 4.62 | 28 |
| A129 | 397.53 | I.A. | 6.21 | 0.45 | 8.34 | 15.7 |
| A130 | 731.34 | I.A. | 53 | 52.7 | 25.4 | 32.5 |
| A131 | 1106.22 | I.A. | 20.4 | 42.9 | 6.3 | 11.8 |
| A132 | 2518.16 | I.A. | 34.6 | 47.1 | 8.42 | 15.1 |
| A133 | 2014.31 | I.A. | 0.98 | 1.42 | 0.15 | 0.61 |
| A134 | 849.38 | I.A. | 2.18 | 13.6 | 13.5 | 19.3 |
| A135 | 692.06 | I.A. | 56.5 | 57.9 | 32.1 | 67.1 |
| A136 | 2412.51 | I.A. | 2.63 | 15.7 | 0.44 | 15.6 |
| A137 | 948.04 | I.A. | 1.78 | 24.2 | 2.57 | 4.35 |
| A138 | 2572.54 | I.A. | 25.2 | 37.2 | 6.25 | 25.1 |
| A139 | 686.69 | I.A. | 26 | 41.9 | 10.7 | 30.1 |
| A140 | 417.88 | I.A. | 16.1 | 12.8 | 8.54 | 14 |
| A141 | 920.54 | I.A. | 0.97 | 2.9 | 0.94 | 2.77 |
| A142 | 16259.19 | I.A. | 60.5 | 59.4 | 29.6 | 20.9 |
| A143 | 1005.46 | 11900.21 | 0.06 | 6.14 | 0.01 | 0.16 |
| A144 | 15.93 | I.A. | 20.2 | 34 | 10.7 | 36.4 |
| A145 | 13.52 | I.A. | 25.1 | 46 | 18 | 41.5 |
| A146 | 39.31 | I.A. | 34.8 | 54.1 | 21.6 | 49.5 |
| A147 | 469.09 | 3476.39 | 5.61 | 2.95 | 0.94 | 8.6 |
| A148 | 1655.8 | 11119.4 | 4.79 | 28.3 | 0.84 | 7.55 |
| A149 | 2538.22 | 4392.63 | 46 | 17.1 | 9.8 | 25.9 |
| A150 | 3266.86 | I.A. | 36.3 | 51.6 | 0.71 | 17.4 |
| A151 | 1386.57 | I.A. | 15.3 | 38.3 | 0.95 | 7.69 |
| A152 | 13198 | I.A. | 48.9 | 24.1 | 28.1 | 44.4 |
| A153 | 1015.69 | I.A. | 1.09 | 19.8 | 2.49 | 1.88 |
| A154 | 1519.17 | I.A. | 18.3 | 16 | 0.22 | 1.29 |
| A155 | 2859.38 | I.A. | 16.9 | 8.23 | 0.17 | 1.07 |
| A156 | 2955.05 | I.A. | 38.2 | 34.1 | 1.75 | 10.3 |
| A157 | 1466.52 | I.A. | 0.84 | 2.44 | 13.2 | 22.9 |
| A158 | 3320.44 | I.A. | 14.5 | 24.2 | 1.84 | 8.23 |
| A159 | 1622.74 | I.A. | 36 | 19.7 | 3.91 | 9.01 |
| A160 | 2668.9 | I.A. | 6.28 | 16.8 | 0.18 | 0.66 |
| A161 | 12432.45 | I.A. | 0.42 | 7.32 | 0.12 | 1.04 |
| A162 | I.A. | I.A. | 7.13 | 8.48 | 0.25 | 1.91 |
| A163 | 713.67 | I.A. | 36.3 | 50.2 | 44.9 | 67.5 |
| A164 | 274.43 | I.A. | 18.8 | 19.4 | 32.3 | 36.5 |
| A165 | 867.16 | I.A. | 30.7 | 59.2 | 47.5 | 42.1 |
| A166 | 2206.79 | I.A. | 63.9 | 78.7 | 72.2 | 64.3 |
| A167 | I.A. | I.A. | 12 | 13 | 0.15 | 0.06 |
| A168 | 2714.27 | I.A. | 0.09 | 0.52 | 0.02 | 0.45 |
| A169 | 7808.36 | 15032.95 | 0.08 | 0.02 | 0.11 | 0.17 |
| A170 | 5708.95 | I.A. | 1.37 | 2.02 | 2.6 | 0.58 |
| A171 | 158.8 | I.A. | 17.3 | 16 | 28.5 | 36.8 |
| A172 | 189.5 | I.A. | 44.9 | 19.7 | 43.3 | 65.3 |
| A173 | 842.16 | 8746.65 | 23.8 | 17.4 | 54.6 | 70.3 |
| A174 | 77.42 | I.A. | 29.4 | 42.5 | 15.4 | 38 |
| A175 | 188.09 | I.A. | 1.57 | 0.32 | 0.22 | 1.38 |
| A176 | 437.69 | I.A. | 2.72 | 18.6 | 0.16 | 1.34 |
| A177 | 16.25 | I.A. | 29.1 | 57.1 | 9.52 | 39.3 |
| A178 | 524.85 | 7403.25 | 70.6 | 51.4 | 70.8 | 72.8 |
| A179 | 70.45 | I.A. | 34.8 | 69.3 | 15.4 | 21.3 |
| A180 | 715.13 | I.A. | 14.4 | 3.14 | 7.35 | 8.99 |
| B1 | I.A. | I.A. | 5.93 | 4.96 | 1.07 | 1.81 |
| B10 | I.A. | I.A. | 9.22 | 2.71 | 0.1 | 1.05 |
| B11 | I.A. | I.A. | 0.12 | 0.29 | 0.1 | 0.37 |
| B12 | I.A. | I.A. | 1.41 | 11 | 4.16 | 3.28 |
| B13 | I.A. | I.A. | 4.96 | 6.37 | 0.6 | 0.48 |
| B14 | | | 2.9 | 3.31 | 6.02 | 3.9 |
| B15 | | | 0.34 | 0.28 | 0.4 | 0.11 |
| B16 | I.A. | I.A. | 196 | 137 | 110 | 662 |
| B18 | I.A. | I.A. | 3.02 | 2.09 | 0.38 | 0.82 |
| B19 | | | 0.83 | 1.18 | 0.03 | 0.05 |
| B2 | I.A. | I.A. | 7.06 | 1.8 | 0.9 | 0.69 |
| B3 | I.A. | I.A. | 5.71 | 16.1 | 0.91 | 1.61 |
| B4 | I.A. | I.A. | 2.21 | 0.35 | 0.37 | 0.15 |
| B5 | I.A. | I.A. | 1.26 | 0.69 | 0.47 | 0.87 |
| B6 | I.A. | I.A. | 10 | 13.2 | 6.93 | 5.85 |
| B7 | 21860.42 | 21399.25 | 12.3 | 2.33 | 0.74 | 1.32 |
| B8 | 24234.74 | I.A. | 5.24 | 7.12 | 0.47 | 0.69 |
| B9 | 15228.33 | 10394.05 | 0.31 | 3.44 | 0.17 | 0.41 |
| B33 | 24108.97 | I.A. | 4.32 | 0.28 | 1.26 | 0.68 |
| B34 | I.A. | I.A. | 0.97 | 0.87 | 1.77 | 0.75 |
| B35 | I.A. | I.A. | 2.61 | 0.46 | 1.04 | 2.88 |
| B36 | 7025.9 | 7330.58 | 9.8 | 2.95 | 3.93 | 0.7 |
| B37 | I.A. | I.A. | 2.42 | 6.38 | 0.13 | 0.13 |
| B38 | 7559.36 | 8930.37 | 6.78 | 1.03 | 0.69 | 0.54 |
| B39 | 10240.21 | 13116.88 | 16.5 | 5.38 | 0.47 | 0.27 |
| B40 | I.A. | I.A. | 5.73 | 0.73 | 2.25 | 0.48 |
| B41 | I.A. | I.A. | 0.36 | 0.15 | 0.44 | 0.18 |
| B42 | I.A. | I.A. | 26.5 | 8.69 | 11.3 | 6.13 |
| B43 | I.A. | I.A. | 1.19 | 1.88 | 0.92 | 1.83 |
| B44 | I.A. | I.A. | 4.87 | 1.3 | 0.36 | 0.24 |
| B45 | 19911.28 | >24220.76 | 23.2 | 16.3 | 0.19 | 0.1 |
| B47 | I.A. | I.A. | 30.8 | 0.41 | 1.45 | 2.81 |
| B48 | I.A. | I.A. | 1.17 | 1.44 | 1.39 | 8.85 |
| B49 | 11196.16 | >19067.55 | 12.5 | 4.13 | 1.51 | 1.53 |
| B50 | 18821.45 | >24633.33 | 0.94 | 0.06 | 0.74 | 1.21 |
| B51 | I.A. | I.A. | 6.7 | 1.43 | 0.27 | 0.21 |
| B52 | 15879.18 | I.A. | 32.1 | 4.04 | 10.8 | 5.82 |
| B53 | 11226.96 | I.A. | 14.5 | 2.11 | 1.39 | 5.98 |
| B54 | 13892.21 | >23649.10 | 4.16 | 4.57 | 2.69 | 0.4 |
| B55 | >20763.76 | I.A. | 1.7 | 0.3 | 0.4 | 0.41 |
| B56 | I.A. | I.A. | 1.72 | 0.57 | 0.27 | 0.46 |
| B57 | 16294.25 | >24186.65 | 17.3 | 34.4 | 2.84 | 17.5 |
| B58 | 15373.16 | 18362.44 | 2.14 | 1.67 | 5.89 | 0.28 |
| B59 | 7913.66 | 11944.61 | 0.64 | 0.55 | 0.17 | 0.32 |
| B60 | 12755.95 | 15815.22 | 0.45 | 1.16 | 2.65 | 0.33 |
| B61 | I.A. | I.A. | 9.41 | 12.3 | 0.5 | 0.96 |
| B62 | >24319.48 | I.A. | 11.5 | 2.51 | 2.64 | 0.2 |
| B63 | I.A. | I.A. | 0.49 | 1.14 | 0.09 | 6.56 |
| B64 | 15716.46 | >22248.52 | 0.29 | 0.46 | 0.12 | 0.37 |
| B65 | 24500.42 | I.A. | 7.82 | 4.75 | 0.08 | 0.18 |
| B66 | 13355.05 | I.A. | 0.11 | 0.18 | 0.35 | 0.09 |
| B67 | 21024 | 19700.26 | 2.2 | 1.32 | 0.13 | 0.16 |
| B68 | 10106.29 | 14586.01 | 14 | 19.9 | 0.07 | 0.5 |
| B69 | I.A. | I.A. | 0.32 | 0.28 | 0.04 | 0.01 |
| B70 | I.A. | I.A. | 2.19 | 1.37 | 0.19 | 0.35 |
| B71 | I.A. | I.A. | 0.16 | 0.1 | 0.03 | 0.03 |
| B72 | I.A. | I.A. | 0.03 | 0.16 | 0.06 | 0.95 |
| B73 | I.A. | I.A. | 1.62 | 3.87 | 0.48 | 0.81 |
| B74 | 5289.26 | 5491.06 | 3.41 | 0.43 | 0.13 | 0.44 |
| B75 | 7156.54 | 6752.18 | 0.94 | 0.27 | 0.23 | 2.26 |
| B76 | I.A. | I.A. | 0.37 | 0.52 | 0.25 | 0.76 |
| B77 | I.A. | I.A. | 2.21 | 1.67 | 0.59 | 1.11 |
| B78 | I.A. | I.A. | | | | |
| B79 | I.A. | I.A. | | | | |
| B80 | 16160.88 | 19009.7 | | | | |
| B81 | 16926.27 | I.A. | 11.7 | 9.66 | 0.06 | 0.58 |
| B83 | 11344.74 | 15306.85 | 11.1/10.1 | 6.87/5.01 | 0.25/0.24 | 2.94/2.65 |
| B84 | 6848.17 | 19102.09 | 50.1/71.4 | 22/44.8 | 5.77/6.74 | 10.6/16.9 |
| B85 | I.A. | I.A. | 0.8 | 2.02 | 0.13 | 1.26 |
| B86 | 16480.14 | 16661.92 | 12.5 | 17 | 0.11 | 0.27 |
| B87 | 9923.61 | 14056.01 | 1.23 | 0.18 | 0.04 | 0.15 |
| B88 | I.A. | I.A. | 3.15 | 2.29 | 15.2 | 15.6 |
| B89 | I.A. | I.A. | 2.24 | 1.11 | 0.17 | 0.4 |
| B90 | 22817.83 | I.A. | 10.8 | 5.78 | 0.54 | 0.87 |
| B91 | I.A. | I.A. | 33.9 | 44.2 | 0.24 | 0.22 |
| B92 | 22766.57 | I.A. | 6.38 | 2.02 | 0.04 | 0.19 |
| B93 | 20918.76 | I.A. | 12.7 | 11.5 | 0.03 | 0.85 |
| B94 | I.A. | I.A. | 20 | 31.1 | 0.06 | 0.55 |
| B95 | I.A. | I.A. | 0.08 | 0.54 | 0.03 | 0.04 |
| B96 | 20673.15 | I.A. | 8.33 | 13.6 | 0.19 | 0.79 |
| B97 | I.A. | I.A. | 13.8 | 10.8 | 0.13 | 0.3 |
| B98 | I.A. | I.A. | 6.81 | 6.91 | 0.72 | 0.2 |
| B99 | I.A. | I.A. | 49.5 | 49.7 | 2.99 | 4.91 |
| B100 | 22360.78 | I.A. | 1.07 | 1.36 | 0.31 | 0.18 |
| B101 | 15303.64 | 19513.25 | 6.67 | 2.51 | 0.25 | 0.38 |
| B102 | 21329.46 | I.A. | 9.83 | 6.8 | 0.85 | 0.71 |
| B103 | I.A. | I.A. | 28.7 | 19.5 | 1.64 | 1.87 |
| B104 | I.A. | I.A. | 4.41 | 2.24 | 0.14 | 0.21 |
| B105 | I.A. | I.A. | 4.32 | 2.65 | 0.05 | 0.11 |
| B106 | I.A. | I.A. | 0.95 | 5.01 | 0.06 | 0.59 |
| B107 | I.A. | I.A. | 35.4 | 18.8 | 0.33 | 0.55 |
| B108 | I.A. | I.A. | 55.7 | 23.9 | 0.69 | 0.43 |
| B109 | I.A. | I.A. | 60.2 | 60.4 | 5.04 | 10.2 |
| B110 | I.A. | I.A. | 44.9 | 32.4 | 0.51 | 0.38 |
| B111 | I.A. | I.A. | 61.7 | 75.4 | 5.79 | 9.45 |
| B112 | I.A. | I.A. | 66.5 | 41.8 | 1.11 | 1.7 |
| B113 | I.A. | I.A. | 68.2 | 69.7 | 3.11 | 7.08 |
| B114 | I.A. | I.A. | 59.3 | 27.3 | 43.4 | 30.7 |
| B115 | I.A. | I.A. | 70.7 | 30 | 11.5 | 12.8 |
| B116 | 8454.34 | 9477.39 | 11.3 | 8.66 | 0.16 | 0.16 |
| B117 | 16860.96 | 21223.72 | 15.8 | 4.95 | 0.22 | 0.29 |
| B118 | I.A. | I.A. | 40.4 | 14.9 | 0.21 | 0.37 |
| B119 | 13134.66 | 15584.73 | 2.37 | 0.7 | 0.1 | 0.15 |
| B120 | I.A. | I.A. | 6.6 | 2.69 | 0.15 | 0.24 |
| C1 | 24074.63 | I.A. | 23.2 | 7.64 | 0.35 | 29.3 |
| C10 | 24302.71 | I.A. | 29.5 | 38.8 | 9.66 | 22.6 |
| C11 | I.A. | I.A. | 52.3 | 83.1 | 76.1 | 65.2 |
| C12 | I.A. | I.A. | 60.4 | 76.3 | 90.9 | 75.1 |
| C13 | I.A. | I.A. | 64.9 | 87.9 | 81.3 | 82.1 |
| C14 | I.A. | I.A. | 57.8 | 78.7 | 90.3 | 74.3 |
| C15 | I.A. | I.A. | 31.7 | 18.3 | 22.4 | 29.3 |
| C16 | I.A. | I.A. | 26.7 | 3.26 | 78.9 | 8.13 |
| C17 | I.A. | I.A. | 9.27 | 10.3 | 30.9 | 58.7 |
| C18 | | | 0.15 | 0.2 | 0.08 | 0.07 |
| C19 | | | 3.48 | 3.9 | 8.4 | 3.59 |
| C2 | I.A. | I.A. | 18 | 17.6 | 0.23 | 33.9 |
| C20 | | | 4.75 | 10.6 | 15 | 15.3 |
| C21 | I.A. | I.A. | 60.5 | 12.2 | 49 | 64 |
| C23 | 24144.72 | I.A. | 12.7 | 8.52 | 4.38 | 15.8 |
| C27 | 23940.96 | I.A. | 4.86 | 1.26 | 3.74 | 3.25 |
| C29 | I.A. | I.A. | 37.5 | 17 | 41.9 | 46.1 |
| C3 | 16332.93 | I.A. | 9.29 | 4.35 | 8.3 | 16 |
| C30 | I.A. | I.A. | 37.6 | 9.91 | 5.63 | 39.8 |
| C31 | I.A. | I.A. | 38.7 | 5.84 | 1.3 | 53.4 |
| C32 | I.A. | I.A. | 28.6 | 14.6 | 21.4 | 49.7 |
| C33 | 23794.36 | I.A. | 41.1 | 20.3 | 65 | 54.7 |
| C37 | I.A. | I.A. | 26 | 0.08 | 8.58 | 6.44 |
| C39 | I.A. | I.A. | 64.7 | 56.6 | 53.9 | 79.7 |
| C4 | I.A. | I.A. | 49.1 | 62.1 | 70.3 | 96.7 |
| C49 | I.A. | I.A. | 29.1 | 2.9 | 9.17 | 1.94 |
| C5 | I.A. | I.A. | 65.6 | 67.5 | 63 | 87.9 |
| C50 | I.A. | I.A. | 54.5 | 15.1 | 33.4 | 33.8 |
| C53 | 1900.67 | I.A. | 62.6 | 60.2 | 23.6 | 63.8 |
| C54 | 6525 | I.A. | 99.1 | 67.6 | 46.8 | 77.9 |
| C55 | 20126.41 | I.A. | 22.7 | 22.5 | 6.6 | 26.7 |
| C56 | I.A. | I.A. | 21 | 10.9 | 2.02 | 24.4 |
| C57 | 23315.89 | I.A. | 9.54 | 0.43 | 3.43 | 4.2 |
| C58 | I.A. | I.A. | 17 | 34.7 | 27.4 | 35.7 |
| C59 | I.A. | I.A. | 3.23 | 11.5 | 2.48 | 0.87 |
| C6 | 11826.34 | 15282.48 | 31.1 | 8.19 | 7.86 | 32.8 |
| C60 | I.A. | I.A. | 0.25 | 6.4 | 0.09 | 0.05 |
| C61 | I.A. | I.A. | 0.16 | 3.4 | 0.03 | 0.27 |
| C62 | I.A. | I.A. | 35.4 | 8.75 | 0.74 | 53 |
| C7 | I.A. | I.A. | 26.6 | 27.6 | 10.4 | 39.7 |
| C8 | I.A. | I.A. | 1.66 | 8.76 | 1.37 | 5.88 |
| C9 | I.A. | I.A. | 0.81 | 8.24 | 0.38 | 3.68 |
| C84 | I.A. | I.A. | 22.3 | 4.2 | 0.28 | 31.5 |
| C85 | 6118.7 | I.A. | 21.3 | 11.2 | 0.21 | 1.61 |
| C86 | I.A. | I.A. | 43.5 | 20.2 | 15 | 27.3 |
| C87 | 4046.06 | I.A. | 14.2 | 6.85 | 0.21 | 12.8 |
| C88 | 21782.99 | I.A. | 14.8 | 5.31 | 1.91 | 15.7 |
| C89 | I.A. | I.A. | 16.5 | 5.16 | 0.31 | 24 |
| C90 | I.A. | I.A. | 1.54 | 7.22 | 1.77 | 1.77 |
| C91 | 4207.26 | I.A. | 88.3 | 41.8 | 28.2 | 67.8 |
| C92 | 22024.56 | I.A. | 15.8 | 3.9 | 5.09 | 7.96 |
| C93 | I.A. | I.A. | 16.3 | 1.23 | 6.79 | 3.12 |
| C94 | I.A. | I.A. | 52.1 | 20.7 | 10.6 | 63.4 |
| C95 | 127.32 | 19590.43 | 0.28 | 1.89 | 0.11 | 0.44 |
| C96 | 2962.77 | I.A. | 6.58 | 0.76 | 0.13 | 0.07 |
| C97 | 24974.16/23623.75 | I.A. | 3.9/3.33 | 5.97/7.43 | 12.7/1.61 | 4.81/6.52 |
| C99 | 21852.79 | I.A. | 19.1 | 4.12 | 0.26 | 26.1 |
| C100 | I.A. | I.A. | 19.8 | 13.6 | 18.5 | 19.1 |
| C102 | 3977.79 | 14582.2 | 6.44 | 2.73 | 0.39 | 0.93 |
| C103 | 13990.43 | I.A. | 54.8 | 30 | 12.9 | 34.8 |
| C104 | 6164.62 | I.A. | 4.94 | 0.82 | 0.09 | 3.01 |
| C105 | 3647.32 | I.A. | 1.99 | 1.71 | 0.67 | 0.19 |
| C106 | I.A. | I.A. | 28.8 | 3.54 | 1.96 | 19 |
| C107 | 2161.17 | I.A. | 16.4 | 18.2 | 5.47 | 23.7 |
| C108 | >23833.72/20270.19 | I.A. | 13.1/2.28 | 3.87/7.05 | 2.55/1.11 | 8.71/7.07 |
| C110 | 1780.7 | I.A. | 74.3 | 36 | 22.2 | 61.3 |
| C111 | I.A. | I.A. | 0.64 | 0.62 | 0.28 | 0.86 |
| C112 | 20655.85 | I.A. | 3.03 | 12.6 | 13.1 | 23.6 |
| C113 | 22771.47 | I.A. | 5.06 | 14.8 | 3.66 | 14.9 |
| C114 | I.A. | I.A. | 15.2 | 19.2 | 0.22 | 0.67 |
| C115 | >22758.87 | I.A. | 1.07 | 2.2 | 0.66 | 1.3 |
| C116 | I.A. | I.A. | 5.51 | 6.02 | 6.55 | 24.8 |
| C117 | 1132.24 | I.A. | 7.53 | 16.5 | 4.1 | 9.81 |
| C118 | I.A. | I.A. | 48.1 | 33.5 | 26.9 | 59.7 |
| C119 | I.A. | I.A. | 42.4 | 49.6 | 23.4 | 61.3 |
| C120 | I.A. | I.A. | 38 | 36.8 | 51.1 | 62.8 |
| C121 | I.A. | I.A. | 52.1 | 50.2 | 44.2 | 64.9 |
| C122 | I.A. | I.A. | 50.6 | 52.6 | 52.2 | 75.8 |
| C123 | I.A. | I.A. | 23.6 | 6.16 | 3.11 | 58.5 |
| D2 | >23879.27 | I.A. | 2.76 | 0.39 | 0.23 | 5.79 |
| D3 | 10241.92 | >17792.16 | 0.46 | 0.26 | 0.09 | 0.24 |
| D4 | I.A. | I.A. | 29.8 | 1.86 | 1.35 | 5.39 |
| D5 | 18898.56 | >24698.72 | 0.54 | 0.16 | 0.17 | 0.18 |
| D6 | 8675.14 | 12342.16 | 0.09 | 0.06 | 0.04 | 0.03 |
| D7 | I.A. | I.A. | 0.56 | 2.55 | 0.07 | 0.04 |
| E1 | 23854.73 | I.A. | 0.33 | 0.33 | 0.53 | 0.57 |
| E10 | I.A. | I.A. | 0.46 | 0.39 | 0.06 | 0.37 |
| E11 | I.A. | I.A. | 0.36 | 2.06 | 1.33 | 0.97 |
| E12 | I.A. | I.A. | 0.23 | 0.83 | 0.19 | 0.45 |
| E13 | 5905.34 | I.A. | 1.95 | 10.1 | 0.67 | 5.08 |
| E14 | I.A. | I.A. | 0.1 | 0.11 | 0.11 | 0.2 |
| E15 | I.A. | I.A. | 0.54 | 0.53 | 0.11 | 0.91 |
| E16 | I.A. | I.A. | 37 | 51.2 | 0.72 | 1.56 |
| E17 | I.A. | I.A. | 35.8 | 27.8 | 18.1 | 35.6 |
| E18 | I.A. | I.A. | 33.5 | 19.4 | 0.27 | 0.2 |
| E2 | 7338.32 | I.A. | 28.6 | 35.4 | 13.5 | 17.5 |
| E20 | 24593.76 | 23271.91 | 0.82 | 0.14 | 0.62 | 0.96 |
| E21 | 15618.91 | 23684.91 | 0.02 | 0.1 | 0.23 | 0.8 |
| E22 | 11948.19 | I.A. | 1.4 | 0.79 | 0.58 | 11.5 |
| E23 | 18033.22 | I.A. | 6.17 | 4.14 | 2.07 | 3 |
| E26 | 761.86 | I.A. | | | | |
| E3 | I.A. | I.A. | 2.41 | 0.35 | 0.6 | 0.35 |
| E30 | I.A. | I.A. | 26.4 | 37.7 | 9.96 | 20.8 |
| E31 | 8897.49 | 7854.61 | 38.3 | 21.2 | 0.25 | 2.06 |
| E32 | 24907.91 | I.A. | 3.3 | 0.79 | 0.09 | 0.21 |
| E33 | I.A. | I.A. | 2.58 | 0.18 | 0.1 | 1.6 |
| E34 | 19596.75 | 7916.29 | 0.06 | 0.26 | 3.44 | 0.19 |
| E35 | 8641.35 | 18212.52 | 0.08 | 1.2 | 0.7 | 0.41 |
| E36 | I.A. | 13845.23 | 27.8 | 1.49 | 0.83 | 1.63 |
| E37 | I.A. | I.A. | 0.18 | 0.34 | 0.08 | 0.08 |
| E38 | 23787.9 | 22997.79 | 0.25 | 0.33 | 0.13 | 0.1 |
| E39 | 8425.5 | 13879.56 | 0.23 | 1.17 | 0.19 | 0.34 |
| E4 | I.A. | I.A. | 0.27 | 0.19 | 0.14 | 0.12 |
| E40 | 6725.83 | I.A. | 2.75 | 1.43 | 0.1 | 0.43 |
| E41 | 7744.88 | 14444.39 | 0.41 | 0.61 | 0.34 | 0.33 |
| E42 | 17348.22 | I.A. | 0.18 | 0.38 | 0.26 | 0.09 |
| E43 | 16734.57 | 20323.22 | 0.5 | 0.9 | 0.35 | 0.28 |
| E44 | 22892.41 | I.A. | 0.24 | 0.72 | 0.59 | 0.5 |
| E45 | 17252.87 | 14283.69 | 2.25 | 1.2 | 0.23 | 0.19 |
| E46 | 3650.74 | I.A. | 22.1 | 1.85 | 0.6 | 7.36 |
| E47 | 10374.58 | 13889.33 | 0.55 | 2.13 | 5.23 | 0.57 |
| E48 | I.A. | I.A. | 2.74 | 0.45 | 0.12 | 0.93 |
| E49 | | | 1.59 | 1.92 | 0.09 | 0.09 |
| E5 | 8089.21 | 19631.02 | 16.7 | 46.1 | 2.47 | 5.77 |
| E50 | I.A. | I.A. | 0.23 | 0.64 | 0.08 | 0.06 |
| E51 | I.A. | I.A. | 0.08 | 0.98 | 0.06 | 0.01 |
| E52 | I.A. | I.A. | 1.28 | 9.09 | 0.32 | 0.44 |
| E53 | I.A. | I.A. | 5 | 5.58 | 2.89 | 7.99 |
| E54 | | | 1.05 | 6.61 | 0.17 | 0.37 |
| E56 | 21506.49 | I.A. | | | | |
| E57 | 6896.97 | 9509.66 | | | | |
| E58 | I.A. | I.A. | 8.68 | 1.33 | 7.34 | 26.7 |
| E59 | 15212.64 | I.A. | 0.39 | 0.83 | 0.56 | 0.22 |
| E6 | 24174.45 | I.A. | 1.82 | 0.75 | 0.57 | 0.67 |
| E60 | 23632.13 | I.A. | 6.59 | 2.04 | 0.19 | 1.3 |
| E7 | I.A. | I.A. | 0.38 | 0.28 | 0.23 | 0.1 |
| E8 | I.A. | I.A. | 0.6 | 2.86 | 0.02 | 0.01 |
| E9 | I.A. | I.A. | 19 | 4.69 | 2.42 | 0.86 |
| E62 | 125.64 | I.A. | 1.51 | 15.4 | 0.19 | 0.69 |
| E63 | 2972.03 | I.A. | 0.72 | 0.23 | 0.32 | 0.02 |
| E64 | 3102.23 | I.A. | 0.21 | 0.16 | 7.11 | 6.7 |
| E65 | 142.18 | I.A. | 0.33 | 2.87 | 0.13 | 0.33 |

I.A. indicates IC50 > 25000

### Embodiments

1. A compound, or pharmaceutically acceptable salt thereof, having a structure of formula (I-A) or (I′-A):

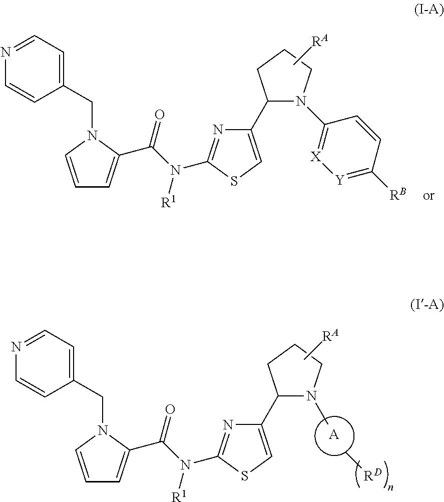

wherein
R¹ is H, C₁₋₃alkyl, or SO₂C₁₋₆alkyl;
each of X and Y is independently N or CR^{C};
ring A is a 6-membered heteroaryl having 2 nitrogen ring atoms;
R^{A} is H, C₁₋₆alkyl, OR^{N}, N(R^{N})₂, OC₁₋₆alkylene-N(R^{N})₂, or OC₁₋₆alkylene-OR^{N};
R^{B} is C₁₋₆alky, C₁₋₆alkoxy, C₁₋₃alkylene-C₁₋₃alkoxy, C₁₋₆haloalkyl, C₁₋₆hydroxyalkyl, halo, C₃₋₆cycloalkyl, CO₂R^{N}, C₀₋₃alkylene-N(R^{N})₂, NO₂, C₀₋₃alkylene-C(O)N(R^{N})₂, C₀₋₃alkylene-N(R^{N})C(O)R^{N}, Het, or OHet,
Het is an aromatic or non-aromatic 4-7 membered heterocycle having 1-3 ring heteroatoms selected from N, 0, and S, and Het is optionally substituted with 1 substituent selected from C₁₋₆alkyl, C₁₋₆alkoxy, oxo, C(O)R^{N}, and SO₂R^{N};
each R^{N} is independently H or C₁₋₆alkyl;
each R^{C} is independently H, halo, C₁₋₆alkoxy, or C₁₋₆alkyl;
n is 0, 1, or 2;
each R^{D}, when present, is independently halo, C₁₋₆alkoxy, or C₁₋₆alkyl; and
each R^{N} is independently H or C₁₋₆alkyl,
   with the proviso that when R¹ is H, X and Y are each CR^{C}, and at least one R^{C} is F, then R^{B} is not F.

2. The compound or salt of embodiment 1, wherein R¹ is H.

3. The compound or salt of embodiment 1 or 2, wherein R^{A} is H.

4. The compound or salt of embodiment 1 or 2, wherein R^{A} is OC₁₋₆alkylene-N(R^{N})₂ or OC₁₋₆alkylene-OR^{N}.

5. The compound or salt of embodiment 1 or 2, wherein R^{A} is OR^{N} or N(R^{N})₂.

6. The compound or salt of any one of embodiments 1 to 5, wherein X is N.

7. The compound or salt of any one of embodiments 1 to 5, wherein X is CR^{C}.

8. The compound or salt of any one of embodiments 1 to 7, wherein Y is N.

9. The compound or salt of any one of embodiments 1 to 7, wherein Y is CR^{C}.

10. The compound or salt of embodiment 7 or 9, wherein at least one R^{C} is H.

11. The compound or salt of embodiment 10, wherein each R^{C} is H.

12. The compound or salt of embodiment 7, 9, or 10, wherein at least one R^{C} is halo.

13. The compound or salt of embodiment 12, wherein R^{C} is fluoro.

14. The compound or salt of embodiment 7, 9, 10, 12, or 13, wherein at least one R^{C} is C₁₋₆ alkoxy or C₁₋₆alkyl.

15. The compound or salt of any one of embodiments 1 to 14, wherein R^{B} is C₁₋₆alkyl.

16. The compound or salt of any one of embodiments 1 to 14, wherein R^{B} is C₁₋₆haloalkyl, C₁₋₆hydroxyalkyl, or halo.

17. The compound or salt of any one of embodiments 1 to 14, wherein R^{B} is CO₂R^{N}, C₀₋₃alkylene-N(R^{N})₂, C₀₋₃alkylene-C(O)N(R^{N})₂, or C₀₋₃alkylene-N(R^{N})C(O)R^{N}.

18. The compound or salt of any one of embodiments 1 to 14, wherein R^{B} is C₃₋₆cycloalkyl, Het, or OHet.

19. The compound or salt of embodiment 18, wherein Het is an aromatic 5-7 membered heterocycle having 1-3 ring heteroatoms.

20. The compound or salt of embodiment 19, wherein Het is imidazole or oxazole.

21. The compound or salt of embodiment 18, wherein Het is a non-aromatic 4-7 membered heterocycle having 1-3 ring heteroatoms.

22. The compound or salt of embodiment 21, wherein Het is tetrahydropyran, piperidine, morpholine, tetrahydrofuran, pyrrolidine, or oxetanyl.

23. The compound or salt of any one of embodiments 18 to 22, wherein Het is unsubstituted.

24. The compound or salt of any one of embodiments 18 to 22, wherein Het is substituted.

25. The compound or salt of embodiment 24, wherein Het is a non-aromatic 4-7 membered heterocycle and is substituted with oxo.

26. The compound or salt of embodiment 24, wherein Het is substituted with C₁₋₆alkyl.

27. The compound or salt of embodiment 24, wherein Het is substituted with C₁₋₆alkoxy.

28. The compound or salt of embodiment 24, wherein Het is substituted with C(O)R^{N} or SO₂R^{N}.

29. The compound or salt of any one of embodiments 1 to 5, wherein ring A is pyrimidinyl.

30. The compound or salt of any one of embodiments 1 to 5, wherein ring A is pyrazinyl.

31. The compound or salt of any one of embodiments 1 to 5, wherein ring A is pyradazinyl.

32. The compound or salt of any one of embodiments 1 to 5 and 29 to 31, wherein n is 0.

33. The compound or salt of any one of embodiments 1 to 5 and 29 to 31, wherein n is 1.

34. The compound or salt of any one of embodiments 1 to 5 and 29 to 31, wherein n is 2.

35. The compound or salt of embodiment 33 or 34, wherein at least one R^{D} is halo.

36. The compound or salt of embodiment 35, wherein R^{D} is fluoro.

37. The compound or salt of any one of embodiments 33 to 36, wherein at least one R^{D} is C₁₋₆alkoxy.

38. The compound or salt of any one of embodiments 33 to 37, wherein at least one R^{D} is C₁₋₆alkyl.

39. The compound or salt of any one of embodiments 1 to 38, wherein each R^{N} is independently H or methyl.

40. The compound or salt of embodiment 1, having a structure as shown in Table A.

41. A compound, or pharmaceutically acceptable salt thereof, having a structure of formula (II-A):

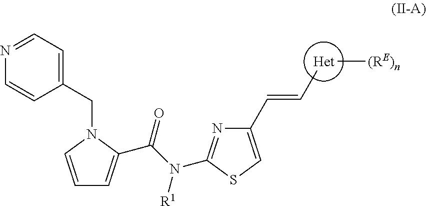

wherein
R¹ is H, C₁₋₃alkyl, or SO₂C₁₋₆alkyl;
Het is oxazole, imidazole, diazinyl, pyrazole, isoxazole, morpholine, tetrahydroquinoline, oxazolidinone, piperidinone, or dihydrooxazole;
n is 0, 1, or 2; and
each R^{E}, when present, is independently halo, C₁₋₆alkyl, phenyl, C(O)N(R^{N})₂, CN, C₀₋₆alkylene-OR^{N}, C₀₋₆alkylene-N(R^{N})₂, C₁₋₆haloalkyl, C₁₋₆haloalkoxy, C₃₋₆cycloalkyl, or CO₂R^{N};
   wherein when R^{E} is phenyl, it is optionally substituted with 1-2 groups independently selected from halo, C₁₋₆alkyl, CN, C₁₋₆haloalkyl, C₁₋₆haloalkoxy, CO₂R^{N}, CON(R^{N})₂, N(R^{N})COR^{N}, and OR^{N}; and
each R^{N} is independently H or C₁₋₆alkyl,
   with the proviso that when Het is diazinyl, n is 1 or 2.

42. The compound or salt of embodiment 41, wherein R¹ is H.

43. The compound or salt of embodiment 41 or 42, wherein Het is oxazole.

44. The compound or salt of embodiment 41 or 42, wherein Het is imidazole.

45. The compound or salt of embodiment 41 or 42, wherein Het is diazinyl.

46. The compound or salt of embodiment 41 or 42, wherein Het is isoxazole, morpholine, tetrahydroquinoline, oxazolidinone, piperidinone, or dihydrooxazole.

47. The compound or salt of any one of embodiments 41 to 46, wherein n is 0.

48. The compound or salt of any one of embodiments 41 to 46, wherein n is 1.

49. The compound or salt of any one of embodiments 41 to 46, wherein n is 2.

50. The compound or salt of embodiment 48 or 49, wherein at least one R^{E} is halo.

51. The compound or salt of embodiment 50, wherein at least one R^{E} is fluoro.

52. The compound or salt of any one of embodiments 48 to 51, wherein at least one R^{E} is C₁₋₆alkyl or C(O)N(R^{N})₂.

53. The compound or salt of any one of embodiments 48 to 52, wherein at least one R^{E} is C₀₋₆alkylene-OR^{N} or C₀₋₆alkylene-N(R^{N})₂.

54. The compound or salt of any one of embodiments 48 to 53, wherein at least one R^{E} is phenyl.

55. The compound or salt of embodiment 54, wherein the phenyl is unsubstituted.

56. The compound or salt of embodiment 54, wherein the phenyl is substituted with 1 substituent selected from halo, C₁₋₆haloalkyl, C₁₋₆haloalkoxy, CON(R^{N})₂, N(R^{N})COR^{N} and OR^{N}.

57. The compound or salt of embodiment 41, having a structure as shown in Table B.

58. A compound, or pharmaceutically acceptable salt thereof, having a structure of formula (III):

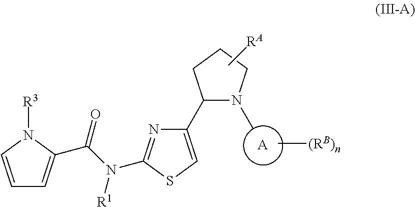

wherein:
R¹ is H, C₁₋₃alkyl, or SO₂C₁₋₆alkyl;
R^{A} is H, C₁₋₆alkyl, OR^{N}, N(R^{N})₂, OC₁₋₆alkylene-N(R^{N})₂, or OC₁₋₆alkylene-OR^{N};
n is 0, 1, or 2;
ring A is phenyl or a 6-membered heteroaryl having 1 or 2 nitrogen ring atoms;
each R^{B}, when present, is independently C₁₋₆alkyl, C₁₋₆alkoxy, C₁₋₆haloalkoxy, C₁₋₃alkylene-C₁₋₃alkoxy, C₁₋₆ haloalkyl, C₁₋₆hydroxyalkyl, halo, C₃₋₆cycloalkyl, CO₂R^{N}, C₀₋₃alkylene-C(O)N(R^{N})₂, N(R^{N})₂, NO₂, C₀₋₃alkylene-N(R^{N})C(O)R^{N}, C₀₋₃alkylene-N(R^{N})C(O)R^{N}, Het, or OHet;
Het is an aromatic or non-aromatic 4-7 membered heterocycle having 1-3 ring heteroatoms selected from N, O, and S;
Het is optionally substituted with 1 substituent selected from C₁₋₆alkyl, C₁₋₆alkoxy, oxo, C(O)R^{N}, and SO₂R^{N};
R³ is C₁₋₆alkylene-X, C₂₋₆alkenylene-X, or C₀₋₂alkylene-C₃₋₆carbocycle-C₀₋₂alkylene-X and the alkylene is optionally substituted with OR^{N};
X is H, OC₁₋₃alkyl, C≡CR^{N}; CN, CO₂R^{N}; CON(R^{N})₂, or Ar,
Ar is a 3-10 membered aromatic or non-aromatic monocyclic or polycyclic ring having 0-4 ring heteroatoms selected from N, O, and S, with the proviso that when Ar is a 6-membered aromatic ring, it has 0 or 2-4 ring heteroatoms,
Ar is optionally substituted with C₁₋₃alkyl, C₀₋₂alklene-CN, CON(R^{N})₂, tetrazole, oxazole, or 1-2 halo; and
each R^{N} is independently H or C₁₋₆alkyl.

59. The compound or salt of embodiment 58, wherein R¹ is H.

60. The compound or salt of embodiment 58 or 59, wherein R^{A} is H.

61. The compound or salt of embodiment 58 or 59, wherein R^{A} is OC₁₋₆alkylene-N(R^{N})₂ or OC₁₋₆alkylene-OR^{N}.

62. The compound or salt of embodiment 58 or 59, wherein R^{A} is OR^{N} or N(R^{N})₂.

63. The compound or salt of any one of embodiments 58 to 62, wherein ring A is phenyl.

64. The compound or salt of any one of embodiments 58 to 62, wherein ring A is a 6-membered heteroaryl having 1 or 2 nitrogen ring atoms.

65. The compound or salt of embodiment 64 wherein ring A is pyridyl.

66. The compound or salt of embodiment 64, wherein ring A is a diazinyl.

67. The compound or salt of embodiment 66, wherein ring A is pyrimidinyl.

68. The compound or salt of embodiment 66, wherein ring A is pyrazinyl.

69. The compound or salt of embodiment 66, wherein ring A is pyradazinyl.

70. The compound or salt of any one of embodiments 58 to 69, wherein n is 0.

71. The compound or salt of any one of embodiments 58 to 69, wherein n is 1.

72. The compound or salt of embodiment 71, wherein R^{B} is C₁₋₆alkyl.

73. The compound or salt of embodiment 71, wherein R^{B} is C₁₋₆haloalkyl, C₁₋₆hydroxyalkyl, or halo.

74. The compound or salt of embodiment 71, wherein R^{B} is CO₂R^{N}, N(R^{N})₂, C₀₋₃alkylene-C(O)N(R^{N})₂, or C₀₋₃alkylene-N(R^{N})C(O)R^{N}.

75. The compound or salt of embodiment 71, wherein R^{B} is C₃₋₆cycloalkyl, Het, or OHet.

76. The compound or salt of embodiment 75, wherein Het is an aromatic 5-7 membered heterocycle having 1-3 ring heteroatoms.

77. The compound or salt of embodiment 75, wherein Het is a non-aromatic 4-7 membered heterocycle having 1-3 ring heteroatoms.

78. The compound or salt of any one of embodiments 75 to 77, wherein Het is unsubstituted.

79. The compound or salt of any one of embodiments 75 to 77, wherein Het is substituted.

80. The compound or salt of embodiment 79, wherein Het is a non-aromatic 4-7 membered heterocycle and is substituted with oxo.

81. The compound or salt of embodiment 79, wherein Het is substituted with C₁₋₆alkyl.

82. The compound or salt of embodiment 79, wherein Het is substituted with C₁₋₆alkoxy.

83. The compound or salt of embodiment 79, wherein Het is substituted with C(O)R^{N} or SO₂R^{N}.

84. The compound or salt of any one of embodiments 58 to 83, wherein R³ is C₁₋₆alkylene-X.

85. The compound or salt of any one of embodiments 58 to 83, wherein R³ C₂₋₆alkenylene-X or C₀₋₂alkylene-C₃₋₆carbocycle-C₀₋₂alkylene-X.

86. The compound or salt of any one of embodiments 58 to 85, wherein X is H, OC₁₋₆alkyl, CN, CO₂R^{N}, or CON(R^{N})₂.

87. The compound or salt of any one of embodiments 58 to 85, wherein X is C≡CR^{N}.

88. The compound or salt of any one of embodiments 58 to 85, wherein X is Ar.

89. The compound or salt of embodiment 88, wherein Ar is 3-10 membered non-aromatic monocyclic or polycyclic ring having 0-4 ring heteroatoms selected from N, O, and S.

90. The compound or salt of embodiment 88, wherein Ar is a 5-10 membered aromatic monocyclic or polycyclic ring having 0-4 ring heteroatoms selected from N, O, and S.

91. The compound or salt of embodiment 90, wherein Ar is phenyl.

92. The compound or salt of embodiment 90, wherein Ar is a 5-10 membered aromatic monocyclic or polycyclic ring having 1-4 ring heteroatoms selected from N, O, and S.

93. The compound or salt of embodiment 90, wherein Ar is a 5 or 7-10 membered aromatic monocyclic or polycyclic ring having 1-4 ring heteroatoms selected from N, O, and S.

94. The compound or salt of embodiment 90, wherein Ar is a 6-10 membered aromatic monocyclic or polycyclic ring having 2-4 ring heteroatoms selected from N, O, and S.

95. The compound or salt of embodiment 90, wherein Ar is phenyl, tetrahydropyran, dihydropyran, tetrahydrofuran, C₃₋₆cycloalkyl, tetrazole, triazole, oxazole, tetrahydroquinoline, N-methyl-tetrahydroisoquinoline, tetrahydrothiopyranyl-dioxide, pyridinone, piperidinone, or oxetanyl.

96. The compound or salt of any one of embodiments 90 to 95, wherein Ar is unsubstituted.

97. The compound or salt of any one of embodiments 90 to 95, wherein Ar is substituted.

98. The compound or salt of embodiment 97, wherein Ar is substituted with C₁₋₃alkyl, C₀₋₂alklene-CN, or CON(R^{N})₂.

99. The compound or salt of embodiment 97 or 98, wherein Ar is substituted with 1 or 2 halo.

100. The compound or salt of embodiment 99, wherein the halo is fluoro.

101. The compound or salt of embodiment 58, having a structure as shown in Table C.

102. A compound, or pharmaceutically acceptable salt thereof, having a structure of formula (IV):

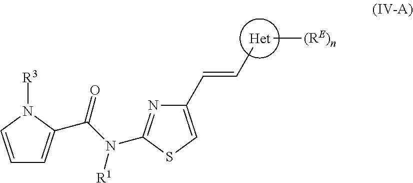

R¹ is H, C₁₋₃alkyl, or SO₂C₁₋₆alkyl;
Het is 3-10 membered aromatic or non-aromatic heterocycle having 1-4 ring heteroatoms selected from N, O, and S;
n is 0, 1, or 2; and
each R^{E}, when present, is independently halo, C₁₋₆alkyl, phenyl, C(O)N(R^{N})₂, CN, C₀₋₆alkylene-OR^{N}, C₀₋₆alkylene-N(R^{N})₂, C₁₋₆haloalkyl, C₁₋₆haloalkoxy, C₃₋₆cycloalkyl, or CO₂R^{N};
   wherein when R^{E} is phenyl, it is optionally substituted with 1-2 groups independently selected from halo, C₁₋₆alkyl, CN, C₁₋₆haloalkyl, C₁₋₆haloalkoxy, CO₂R^{N}, CON(R^{N})₂, N(R^{N})COR^{N}, and OR^{N};
R³ is C₁₋₆alkylene-X, C₂₋₆alkenylene-X, or C₀₋₂alkylene-C₃₋₆carbocycle-C₀₋₂alkylene-X;
X is H, OC₁₋₃alkyl, C≡CR^{N}; CN, CO₂R^{N}; CON(R^{N})₂, or Ar,
Ar is a 3-10 membered aromatic or non-aromatic ring having 0-4 ring heteroatoms selected from N, O, and S, with the proviso that when Ar is a 6-membered aromatic ring, it has 0 or 2-4 ring heteroatoms;
Ar is optionally substituted with C₁₋₃alkyl, C₀₋₂alklene-CN, CON(R^{N})₂, tetrazole, oxazole, or 1-2 halo; and
each R^{N} is independently H or C₁₋₆alkyl.

103. The compound or salt of embodiment 102, wherein R¹ is H.

104. The compound or salt of embodiment 102 or 103, wherein Het is a 3-10 membered non-aromatic heterocycle having 1-4 ring heteroatoms selected from N, O, and S.

105. The compound or salt of embodiment 104, wherein Het is tetrahydropyran.

106. The compound or salt of embodiment 102 or 103, wherein Het is a 5-10 membered aromatic heterocycle having 1-4 ring heteroatoms selected from N, O, and S.

107. The compound or salt of embodiment 106, wherein oxazole.

108. The compound or salt of embodiment 106, wherein Het is imidazole.

109. The compound or salt of embodiment 106, wherein Het is diazinyl.

110. The compound or salt of embodiment 109, wherein the diazinyl is pyrimidinyl.

111. The compound or salt of embodiment 109, wherein the diazinyl is pyrazinyl.

112. The compound or salt of embodiment 109, wherein the diazinyl is pyradazinyl.

113. The compound or salt of embodiment 102 or 103, wherein Het is isoxazole, morpholine, tetrahydroquinoline, oxazolidinone, piperidinone, or dihydrooxazole.

114. The compound or salt of any one of embodiments 102 to 113, wherein n is 0.

115. The compound or salt of any one of embodiments 102 to 113, wherein n is 1.

116. The compound or salt of any one of embodiments 102 to 113, wherein n is 2.

117. The compound or salt of embodiment 115 or 116, wherein at least one R^{E} is halo.

118. The compound or salt of embodiment 117, wherein at least one R^{E} is fluoro.

119. The compound or salt of any one of embodiments 115 to 1118, wherein at least one R^{E} is C₁₋₆alkyl or C(O)N(R^{N})₂.

120. The compound or salt of any one of embodiments 115 to 119, wherein at least one R^{E} is C₀₋₆alkylene-OR^{N} or C₀₋₆alkylene-N(R^{N})₂.

121. The compound or salt of any one of embodiments 115 to 120, wherein at least one R^{E} is phenyl.

122. The compound or salt of embodiment 121, wherein the phenyl is unsubstituted.

123. The compound or salt of embodiment 121, wherein the phenyl is substituted with 1 substituent selected from halo, C₁₋₆haloalkyl, C₁₋₆haloalkoxy, CON(R^{N})₂, N(R^{N})COR^{N} and OR^{N}.

124. The compound or salt of any one of embodiments 102 to 123, wherein R³ is C₁₋₆alkylene-X.

125. The compound or salt of any one of embodiments 102 to 123, wherein R³ C₂₋₆alkenylene-X or C₀₋₂alkylene-C₃₋₆carbocycle-C₀₋₂alkylene-X.

126. The compound or salt of any one of embodiments 102 to 125, wherein X is H, OC₁₋₃alkyl, CN, CO₂R^{N}, or CON(R^{N})₂.

127. The compound or salt of any one of embodiments 102 to 125, wherein X is C≡CR^{N}.

128. The compound or salt of any one of embodiments 102 to 125, wherein X is Ar.

129. The compound or salt of embodiment 128, wherein Ar is 3-10 membered non-aromatic monocyclic or polycyclic ring having 0-4 ring heteroatoms selected from N, O, and S.

130. The compound or salt of embodiment 128, wherein Ar is a 5-10 membered aromatic monocyclic or polycyclic ring having 0-4 ring heteroatoms selected from N, O, and S.

131. The compound or salt of embodiment 128, wherein Ar is phenyl.

132. The compound or salt of embodiment 128, wherein Ar is a 5-10 membered aromatic monocyclic or polycyclic ring having 1-4 ring heteroatoms selected from N, O, and S.

133. The compound or salt of embodiment 132, wherein Ar is a 5 or 7-10 membered aromatic monocyclic or polycyclic ring having 1-4 ring heteroatoms selected from N, O, and S.

134. The compound or salt of embodiment 132, wherein Ar is a 6-10 membered aromatic monocyclic or polycyclic ring having 2-4 ring heteroatoms selected from N, O, and S.

135. The compound or salt of embodiment 128, wherein Ar is phenyl, tetrahydropyran, dihydropyran, tetrahydrofuran, C₃₋₆cycloalkyl, tetrazole, triazole, oxazole, tetrahydroquinoline, N-methyl-tetrahydroisoquinoline, tetrahydrothiopyranyl-dioxide, pyridinone, piperidinone, or oxetanyl.

136. The compound or salt of any one of embodiments 128 to 135, wherein Ar is unsubstituted.

137. The compound or salt of any one of embodiments 128 to 135, wherein Ar is substituted.

138. The compound or salt of embodiment 137, wherein Ar is substituted with C₁₋₃alkyl, C₀₋₂alklene-CN, or CON(R^{N})₂.

139. The compound or salt of embodiment 137 or 138, wherein Ar is substituted with 1 or 2 halo.

140. The compound or salt of embodiment 139, wherein the halo is fluoro.

141. The compound or salt of embodiment 102, having a structure as shown in Table D.

142. A compound, or pharmaceutically acceptable salt thereof, as listed in Table E.

143. A pharmaceutical composition comprising the compound or salt of any one of embodiments 1 to 142 and a pharmaceutically acceptable excipient.

144. A method of inhibiting protein secretion in a cell comprising contacting the cell with the compound or salt of any one of embodiments 1 to 142 or the composition of embodiment 143 in an amount effective to inhibit secretion.

145. The method of embodiment 144, wherein the protein is a checkpoint protein.

146. The method of embodiment 144, wherein the protein is a cell-surface protein, endoplasmic reticulum associated protein, or secreted protein involved in regulation of anti-tumor immune response.

147. The method of embodiment 144, wherein the protein is at least one of PD-1, PD-L1, TIM-1, LAG-3, CTLA4, BTLA, OX-40, B7H1, B7H4, CD137, CD47, CD96, CD73, CD40, VISTA, TIGIT, LAIR1, CD160, 2B4, TGFRβ and combinations thereof.

148. The method of embodiment 144, wherein the protein is selected from the group consisting of HER3, TNFα, IL2, and PD1.

149. The method of any one of embodiments 144 to 148, wherein the contacting comprising administering the compound or the composition to a subject in need thereof.

150. A method for treating inflammation in a subject comprising administering to the subject a therapeutically effective amount of the compound or salt of any one of embodiments 1 to 142 or the pharmaceutical composition of embodiment 143.

151. A method for treating cancer in a subject comprising administering to the subject a therapeutically effective amount of the compound or salt of any one of embodiments 1 to 142 or the pharmaceutical composition of embodiment 143.

152. The method of embodiment 151, wherein the cancer is melanoma, multiple myeloma, prostate cancer, lung cancer, pancreatic cancer, squamous cell carcinoma, leukemia, lymphoma, a neuroendocrine tumor, bladder cancer, or colorectal cancer.

153. The method of embodiment 151, wherein the cancer is selected from the group consisting of prostate, lung, bladder, colorectal, and multiple myeloma.

154. The method of embodiment 151, wherein the cancer is non-small cell lung carcinoma, squamous cell carcinoma, leukemia, acute myelogenous leukemia, chronic myelogenous leukemia, lymphoma, NPM/ALK-transformed anaplastic large cell lymphoma, diffuse large B cell lymphoma, neuroendocrine tumors, breast cancer, mantle cell lymphoma, renal cell carcinoma, rhabdomyosarcoma, ovarian cancer, endometrial cancer, small cell carcinoma, adenocarcinoma, gastric carcinoma, hepatocellular carcinoma, pancreatic cancer, thyroid carcinoma, anaplastic large cell lymphoma, hemangioma, or head and neck cancer.

155. The method of embodiment 151, wherein the cancer is a solid tumor.

156. The method of embodiment 151, wherein the cancer is head and neck cancer, squamous cell carcinoma, gastric carcinoma, or pancreatic cancer.

157. A method for treating an autoimmune disease in a subject comprising administering to the subject a therapeutically effective amount of the compound or salt of any one of embodiments 1 to 142 or the pharmaceutical composition of embodiment 143.

158. The method of embodiment 157, wherein the autoimmune disease is psoriasis, dermatitis, systemic scleroderma, sclerosis, Crohn's disease, ulcerative colitis; respiratory distress syndrome, meningitis; encephalitis; uveitis; colitis; glomerulonephritis; eczema, asthma, chronic inflammation; atherosclerosis; leukocyte adhesion deficiency; rheumatoid arthritis; systemic lupus erythematosus (SLE); diabetes mellitus; multiple sclerosis; Reynaud's syndrome; autoimmune thyroiditis; allergic encephalomyelitis; Sjorgen's syndrome; juvenile onset diabetes; tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia; myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia.

159. A method for the treatment of an immune-related disease in a subject comprising administering to the subject a therapeutically effective amount of the compound or salt of any one of embodiments 1 to 142 or the pharmaceutical composition of embodiment 143.

160. The method of embodiment 159, wherein the immune-related disease is rheumatoid arthritis, lupus, inflammatory bowel disease, multiple sclerosis, or Crohn's disease.

161. A method for treating neurodegenerative disease in a subject comprising administering to the subject a therapeutically effective amount of the compound or salt of any one of embodiments 1 to 142 or the pharmaceutical composition of embodiment 143.

162. The method of embodiment 161, wherein the neurodegenerative disease is multiple sclerosis.

163. A method for treating an inflammatory disease in a subject comprising administering to the subject a therapeutically effective amount of the compound or salt of any one of embodiments 1 to 142 or the pharmaceutical composition of embodiment 143.

164. The method of embodiment 163, wherein the inflammatory disease is bronchitis, conjunctivitis, myocarditis, pancreatitis, chronic cholecstitis, bronchiectasis, aortic valve stenosis, restenosis, psoriasis or arthritis.

What is claimed is:

1. A compound, or pharmaceutically acceptable salt thereof, having a structure of formula (I) or (I′): 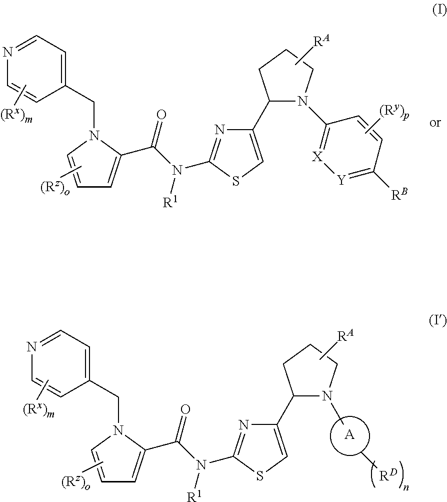
wherein
R¹ is H, C₁₋₃alkyl, or SO₂C₁₋₆alkyl;
each of X and Y is independently N or CR^{C};
ring A is a 6-membered heteroaryl having 2 nitrogen ring atoms;
R^{A} is H, C₁₋₆alkyl, OR^{N}, N(R^{N})₂, OC₁₋₆alkylene-N(R^{N})₂, or OC₁₋₆alkylene-OR^{N};
R^{B} is C₁₋₆alkyl, C₁₋₆alkoxy, C₁₋₃alkylene-C₁₋₃alkoxy, O—C₁₋₃alkylene-C₁₋₃alkoxy, C₁₋₆haloalkyl, C₁₋₆hydroxyalkyl, O—C₁₋₆hydroxyalkyl, halo, C₀₋₃alkylene-CO₂R^{N}, C₀₋₃alkylene-N(R^{N})₂, OC₁₋₃alkylene-N(R^{N})₂, NO₂, C₀₋₃alkylene-C(O)N(R^{N})₂, C₀₋₃alkylene-N(R^{N})C(O)R^{N}, OC₁₋₃alkylene-N(R^{N})C(O)R^{N}, C₀₋₃alkylene-N(R^{N})C(O)N(R^{N})₂, C₀₋₃alkylene-N(R^{N})SO₂R^{N}, C₀₋₃alkylene-N(R^{N})C(O)OR^{N}, C₀₋₃alkylene-OC(O)N(R^{N})₂, C₀₋₃alkylene-Het, C₀₋₃alkylene-OHet, C₀₋₃alkylene-NHCO₂Het, C₀₋₃alkylene-OC(O)Het, C₀₋₃alkylene-N(R^{N})Het or C₀₋₃alkylene-N(R^{N})C(O)Het, or
if
(1) m is 1 or 2;
(2) at least one of X and Y is N,
(3) at least one R^{C} is other than H, or
(4) at least one of o and p is 1,
then R^{B} can be H; or
if Y is CR^{C}, then R^{C} and R^{B} can combine to form a 6-membered fused ring with the carbons to which they are attached having 0-2 ring heteroatoms selected from N, O, and S and optionally substituted with 1 or 2 substituents independently selected from oxo, halo, and C₁₋₆alkyl;
Het is an aromatic or non-aromatic 4-7 membered ring having 0-3 ring heteroatoms selected from N, O, and S, and Het is optionally substituted with 1 or 2 substituents independently selected from C₁₋₆alkyl, halo, OR^{N}, oxo, C(O)R^{N}, C(O)C₃₋₆cycloalkyl, C(O)N(R^{N})₂, SOR^{N}, SO₂R^{N}, and SO₂N(R^{N})₂;
each R^{C} is independently H, halo, C₁₋₆alkoxy, N(R^{N})₂, CN, Het, or C₁₋₆alkyl;
n is 0, 1, or 2;
each R^{D}, when present, is independently halo, C₁₋₆alkoxy, or C₁₋₆alkyl;
m is 0, 1, or 2;
each R^{x}, when present, is independently halo or C₁₋₆alkyl;
p is 0 or 1;
R^{y}, when present, is C₁₋₆alkyl or halo;
o is 0 or 1;
R^{z}, when present, is CN, halo, C(O)N(R^{N})₂, C₁₋₆alkyl, C₁₋₆alkoxy, C₁₋₆hydroxyalkyl, or C₁₋₆haloalkyl; and
each R^{N} is independently H, C₁₋₆alkyl, C₁₋₆hydroxyalkyl, or C₁₋₆haloalkyl,
with the proviso that when each of m, p, and o is 0, R¹ is H, X and Y are each CR^{C}, and at least one R^{C} is F, then R^{B} is not F.

2. The compound or salt of claim 1, wherein R¹ is H.

3. The compound or salt of claim 1 or 2, wherein R^{A} is H.

4. The compound or salt of claim 1 or 2, wherein R^{A} is OC₁₋₆alkylene-N(R^{N})₂ or OC₁₋₆alkylene-OR^{N}.

5. The compound or salt of claim 1 or 2, wherein R^{A} is OR^{N} or N(R^{N})₂.

6. The compound or salt of any one of claims 1 to 5, wherein X is N.

7. The compound or salt of any one of claims 1 to 5, wherein X is CR^{C}.

8. The compound or salt of any one of claims 1 to 7, wherein Y is N.

9. The compound or salt of any one of claims 1 to 7, wherein Y is CR^{C}.

10. The compound or salt of claim 9, wherein R^{C} and R^{B} combine to form a 6-membered fused ring with the carbons to which they are attached having 0-1 ring heteroatoms selected from N, O, and S and optionally substituted with 1 or 2 substituents independently selected from oxo, halo, and C₁₋₆alkyl.

11. The compound or salt of claim 7 or 9, wherein at least one R^{C} is H.

12. The compound or salt of claim 11, wherein each R^{C} is H.

13. The compound or salt of claim 7, 9, or 11, wherein at least one R^{C} is halo.

14. The compound or salt of claim 13, wherein R^{C} is fluoro.

15. The compound or salt of claim 7, 9, 11, 13, or 14, wherein at least one R^{C} is C₁₋₆alkoxy or C₁₋₆alkyl.

16. The compound or salt of claim 7, 9, 11, or 13 to 15, wherein at least one R^{C} is N(R^{N})₂, CN or Het.

17. The compound or salt of any one of claims 1 to 9 and 11 to 16, wherein R^{B} is C₁₋₆alkyl, C₁₋₆alkoxy, C₁₋₃alkylene-C₁₋₃alkoxy, C₁₋₆haloalkyl, C₁₋₆hydroxyalkyl, halo, C₃₋₆cycloalkyl, CO₂R^{N}, C₀₋₃alkylene-N(R^{N})₂, NO₂, C₀₋₃alkylene-C(O)N(R^{N})₂, C₀₋₃alkylene-N(R^{N})C(O)R^{N}, Het, or OHet.

18. The compound or salt of any one of claims 1 to 9 and 11 to 16, wherein R^{B} is C₀₋₃alkylene-N(R^{N})C(O)R^{N}, OC₁₋₃alkylene-N(R^{N})C(O)R^{N}, C₀₋₃alkylene-N(R^{N})C(O)N(R^{N})₂, C₀₋₃alkylene-N(R^{N})C(O)OR^{N}, or C₁₋₆haloalkyl.

19. The compound or salt of any one of claims 1 to 9 and 11 to 16, wherein R^{B} is C₁₋₆alkyl, C₁₋₆haloalkyl, C₁₋₆hydroxyalkyl, or halo.

20. The compound or salt of any one of claims 1 to 9 and 11 to 16, wherein R^{B} is CO₂R^{N}, C₀₋₃alkylene-N(R^{N})₂, C₀₋₃alkylene-C(O)N(R^{N})₂, or C₀₋₃alkylene-N(R^{N})C(O)R^{N}.

21. The compound or salt of any one of claims 1 to 9 and 11 to 16, wherein R^{B} is Het or OHet, and Het is unsubstituted C₃₋₆cycloalkyl, or an aromatic or non-aromatic 4-7 membered heterocycle with 1-3 ring heteroatoms substituted with 1 substituent selected from C₁₋₆alkyl, O—C₁₋₆alkyl, oxo, C(O)C₁₋₆alkyl, and SO₂C₁₋₆alkyl.

22. The compound or salt of any one of claims 1 to 9 and 11 to 16, wherein R^{B} is O—C₁₋₃alkylene-C₁₋₃alkoxy, O—C₁₋₆hydroxyalkyl, OC₁₋₃alkylene-N(R^{N})₂, OC₁₋₃alkylene-N(R^{N})C(O)R^{N}, C₀₋₃alkylene-N(R^{N})C(O)N(R^{N})₂, C₀₋₃alkylene-N(R^{N})SO₂R^{N}, C₀₋₃alkylene-N(R^{N})C(O)OR^{N}, C₁₋₃alkylene-Het, NH-Het, NHC(O)Het, or NHC(O)OHet.

23. The compound or salt of claim 21 or 22, wherein Het is an aromatic 5-7 membered heterocycle having 1-3 ring heteroatoms.

24. The compound or salt of claim 23, wherein Het is imidazole or oxazole.

25. The compound or salt of claim 21 or 22, wherein Het is a non-aromatic 4-7 membered heterocycle having 1-3 ring heteroatoms.

26. The compound or salt of claim 25, wherein Het is tetrahydropyran, piperidine, morpholine, tetrahydrofuran, pyrrolidine, or oxetanyl.

27. The compound or salt of any one of claims 21 to 26, wherein Het is unsubstituted.

28. The compound or salt of any one of claims 21 to 26, wherein Het is substituted.

29. The compound or salt of claim 28, wherein Het is mono-substituted.

30. The compound or salt of claim 28, wherein Het is di-substituted.

31. The compound or salt of any one of claims 28 to 30, wherein Het is a non-aromatic 4-7 membered heterocycle and is substituted with oxo.

32. The compound or salt of any one of claims 28 to 31, wherein Het is substituted with C₁₋₆alkyl.

33. The compound or salt of any one of claims 28 to 32, wherein Het is substituted with C₁₋₆alkoxy.

34. The compound or salt of any one of claims 28 to 33, wherein Het is substituted with C(O)R^{N} or SO₂R^{N}.

35. The compound or salt of any one of claims 28 to 34, wherein Het is substituted with halo.

36. The compound or salt of any one of claims 28 to 35, wherein

37. The compound or salt of any one of claims 28 to 35, wherein C(O)N(R^{N})₂.

38. The compound or salt of any one of claims 1 to 9 and 11 to 15, wherein R^{B} is H.

39. The compound or salt of claim any one of claims 1 to 38, wherein m is 1 and R^{x} is at 2-position of pyridine.

40. The compound or salt of claim 1 to 38, wherein m is 2, optionally where one R^{x} is at 2-position and other R^{x} at 6-position of pyridine.

41. The compound or salt of claim 39 or 40, wherein at least one R^{x} is halo or methyl.

42. The compound or salt of claim 41, wherein halo is fluoro.

43. The compound or salt of any one of claims 38 to 42, wherein X is N.

44. The compound or salt of any one of claims 38 to 43, wherein Y is N.

45. The compound or salt of any one of claims 38 to 44, wherein at least one R^{C} is halo, C₁₋₆alkoxy, N(R^{N})₂, CN, Het, or C₁₋₆alkyl.

46. The compound or salt of claim 45, wherein at least one R^{C} is halo, C₁₋₆alkoxy, or C₁₋₆alkyl.

47. The compound or salt of any one of claims 1 to 46, wherein o is 1, and R^{z} is meta to ring nitrogen.

48. The compound or salt of claim 47, wherein R^{z} is CN, fluoro, or methyl.

49. The compound or salt of any one of claims 1 to 48, wherein p is 1.

50. The compound or salt of claim 49, wherein R^{y} is methyl or fluoro.

51. The compound or salt of any one of claims 1, 17 to 37, 47, and 48, wherein R¹ is H, X and Y are each CH, R^{A} is H, m is 1, R^{x} is fluoro at 2-position of pyridine, and p is 0.

52. The compound or salt of any one of claims 1 to 5, wherein ring A is pyrimidinyl.

53. The compound or salt of any one of claims 1 to 5, wherein ring A is pyrazinyl.

54. The compound or salt of any one of claims 1 to 5, wherein ring A is pyradazinyl.

55. The compound or salt of any one of claims 1 to 5 and 52 to 54, wherein n is 0.

56. The compound or salt of any one of claims 1 to 5 and 52 to 54, wherein n is 1.

57. The compound or salt of any one of claims 1 to 5 and 52 to 54, wherein n is 2.

58. The compound or salt of claim 56 or 57, wherein at least one R^{D} is halo.

59. The compound or salt of claim 58, wherein R^{D} is fluoro.

60. The compound or salt of any one of claims 56 to 59, wherein at least one R^{D} is C₁₋₆alkoxy.

61. The compound or salt of any one of claims 56 to 60, wherein at least one R^{D} is C₁₋₆alkyl.

62. The compound or salt of any one of claims 1 to 61, wherein each R^{N} is independently H or methyl.

63. The compound or salt of any one of claims 1 to 61, wherein at least one R^{N} is C₁₋₆hydroxyalkyl or C₁₋₆haloalkyl.

64. The compound or salt of claim 1, having a structure as shown in Table A.

65. A compound, or pharmaceutically acceptable salt thereof, having a structure of formula (II): 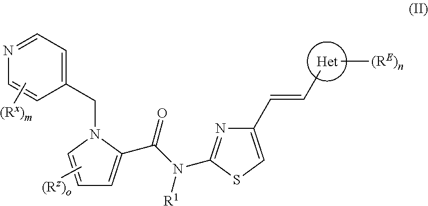
wherein
R¹ is H, C₁₋₃alkyl, or SO₂C₁₋₆alkyl;
Het is oxazole, imidazole, pyrazole, isoxazole, morpholine, tetrahydroquinoline, oxazolidinone, piperidinone, dihydrooxazole, pyrazine, pyrimidine, imidazo[1,2-a]pyridine, 5,6,7,8-tetrahydroimidazo[1,5-a]pyridine, pyridine-2(1H)-one, 6,7-dihydro-5H-pyrrolo[1,2-a]imidazole, or quinoline, or
when at least one of n and m is 1 or 2, Het can be pyridine, and when n is 1 or 2, Het can be diazinyl;
n is 0, 1, or 2;
each R^{E}, when present, is independently halo, C₁₋₆alkyl, C₀₋₆alkylene-C(O)N(R^{N})₂, C₀₋₆alkylene-N(R^{N})C(O)R^{N}, C₀₋₆alkylene-CN, C₀₋₆alkylene-OR^{N}, C₀₋₆alkylene-N(R^{N})₂, C₁₋₆haloalkyl, C₁₋₆haloalkoxy, C₁₋₆hydroxyalkyl, C₀₋₆alkylene-CO₂R^{N}, or C₀₋₆alkylene-[C(O)]₀₋₁-3-6 membered aromatic or non-aromatic ring having 0-2 ring heteroatoms independently selected from N, O and S;
wherein when R^{E} comprises a 3-6 membered ring, it is optionally substituted with 1-2 groups independently selected from halo, C₁₋₆alkyl, CN, C₁₋₆haloalkyl, CO₂R^{N}, C(O)R^{N}, CON(R^{N})₂, N(R^{N})COR^{N}, and OR^{N};
m is 0, 1, or 2;
each R^{x}, when present, is independently halo or C₁₋₆alkyl;
o is 0 or 1;
R^{z}, when present, is CN, halo, C(O)N(R^{N})₂, C₁₋₆alkyl, C₁₋₆alkoxy, C₁₋₆hydroxyalkyl, or C₁₋₆haloalkyl; and
each R^{N} is independently H, C₁₋₆alkyl, C₁₋₆hydroxyalkyl, or C₁₋₆haloalkyl.

66. The compound or salt of claim 65, wherein R¹ is H.

67. The compound or salt of claim 65 or 66, wherein Het is oxazole.

68. The compound or salt of claim 65 or 66, wherein Het is imidazole.

69. The compound or salt of claim 65 or 66, wherein Het is isoxazole, morpholine, tetrahydroquinoline, oxazolidinone, piperidinone, or dihydrooxazole.

70. The compound or salt of claim 65 or 66, wherein Het is pyrazine, pyrimidine, imidazo[1,2-a]pyridine, 5,6,7,8-tetrahydroimidazo[1,5-a]pyridine, pyridine-2(1H)-one, 6,7-dihydro-5H-pyrrolo[1,2-a]imidazole, or quinoline.

71. The compound or salt of any one of claims 65 to 70, wherein n is 0.

72. The compound or salt of any one of claims 65 to 70, wherein n is 1.

73. The compound or salt of any one of claims 65 to 70, wherein n is 2.

74. The compound or salt of claim 72 or 73, wherein Het is diazinyl.

75. The compound or salt of claim 72 or 73, wherein Het is pyridine.

76. The compound or salt of any one of claims 72 to 75, wherein at least one R^{E} is halo.

77. The compound or salt of claim 76, wherein at least one R^{E} is fluoro.

78. The compound or salt of any one of claims 72 to 77, wherein at least one R^{E} is C₁₋₆alkyl or C(O)N(R^{N})₂.

79. The compound or salt of any one of claims 72 to 78, wherein at least one R^{E} is C₀₋₆alkylene-OR^{N} or C₀₋₆alkylene-N(R^{N})₂.

80. The compound or salt of any one of claims 72 to 79, wherein at least one R^{E} is C₁₋₆alkylene-C(O)N(R^{N})₂, C₁₋₆alkylene-CN, C₁₋₆hydroxyalkyl, C₀₋₆alkylene-[C(O)]₀₋₁-3-6 membered non-aromatic ring having 1 or 2 ring heteroatoms independently selected from N, O and S, or C₁₋₆alkylene-CO₂R^{N}.

81. The compound or salt of claim 80, wherein the 3-6 membered ring is unsubstituted.

82. The compound or salt of claim 80, wherein the 3-6 membered ring is substituted.

83. The compound or salt of claim 82, wherein the 3-6 membered ring is substituted with one substituent selected from halo, C₁₋₆alkyl, CN, C₁₋₆haloalkyl, C₁₋₆haloalkoxy, OH, C₁₋₆alkoxy, CO₂R^{N}, C(O)R^{N}, CON(R^{N})₂, and N(R^{N})COR^{N}.

84. The compound or salt of any one of claims 72 to 79, wherein at least one R^{E} is C₀₋₃alkylene-phenyl.

85. The compound or salt of claim 84, wherein the phenyl is unsubstituted.

86. The compound or salt of claim 84, wherein the phenyl is substituted with 1 substituent selected from halo, C₁₋₆haloalkyl, C₁₋₆haloalkoxy, CON(R^{N})₂, N(R^{N})COR^{N} and OR^{N}.

87. The compound or salt of any one of claims 65 to 86, wherein m is 0.

88. The compound or salt of any one of claims 65 to 86, wherein m is 1 and R^{x} is at 2-position of pyridine.

89. The compound or salt of any one of claims 65 to 86, wherein m is 2, optionally where one R^{x} is at 2-position and other R^{x} at 6-position of pyridine.

90. The compound or salt of claim 88 or 89, wherein at least one R^{x} is methyl or fluoro.

91. The compound or salt of any one of claims 88 to 90, wherein Het is pyridine.

92. The compound or salt of any one of claims 65 to 91, wherein o is 0.

93. The compound or salt of any one of claims 65 to 91, wherein o is 1.

94. The compound or salt of claim 93, wherein R^{z} is methyl or fluoro.

95. The compound or salt of any one of claims 65 to 94, wherein each R^{N} is independently H or methyl.

96. The compound or salt of any one of claims 65 to 94, wherein at least one R^{N} is C₁₋₆hydroxyalkyl or C₁₋₆haloalkyl.

97. The compound or salt of claim 65, having a structure as shown in Table B.

98. A compound, or pharmaceutically acceptable salt thereof, having a structure of formula (III): 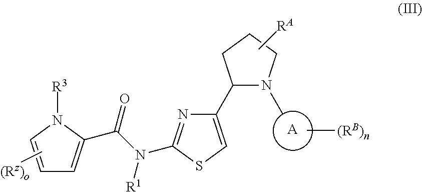
wherein
R¹ is H, C₁₋₃alkyl, or SO₂C₁₋₆alkyl;
R^{A} is H, C₁₋₆alkyl, OR^{N}, N(R^{N})₂, OC₁₋₆alkylene-N(R^{N})₂, or OC₁₋₆alkylene-OR^{N};
n is 0, 1, or 2;
ring A is phenyl or a 6-membered heteroaryl having 1 or 2 nitrogen ring atoms;
each R^{B}, when present, is independently C₁₋₆alkyl, C₁₋₆alkoxy, C₁₋₆haloalkoxy, C₁₋₃alkylene-C₁₋₃alkoxy, C₁₋₆ haloalkyl, C₁₋₆hydroxyalkyl, halo, C₀₋₃alkylene-CO₂R^{N}, C₀₋₃alkylene-C(O)N(R^{N})₂, C₀₋₃alkylene-N(R^{N})₂, OC₁₋₃alkylene-N(R^{N})₂, NO₂, C₀₋₃alkylene-N(R^{N})C(O)R^{N}, C₀₋₃alkylene-N(R^{N})C(O)OR^{N}, OC₁₋₃alkylene-N(R^{N})C(O)R^{N}, C₀₋₃alkylene-N(R^{N})C(O)N(R^{N})₂, C₀₋₃alkylene-N(R^{N})SO₂R^{N}, C₀₋₃alkylene-OC(O)N(R^{N})₂, C₀₋₃alkylene-Het, C₀₋₃alkylene-OHet, C₀₋₃alkylene-NHCO₂Het, C₀₋₃alkylene-OC(O)Het, C₀₋₃alkylene-N(R^{N})Het or C₀₋₃alkylene-N(R^{N})C(O)Het;
Het is an aromatic or non-aromatic 4-7 membered ring having 0-3 ring heteroatoms selected from N, O, and S;
Het is optionally substituted with 1 substituent selected from C₁₋₆alkyl, OR^{N}, halo, oxo, C(O)R^{N}, C(O)N(R^{N})₂, SOR^{N}, SO₂N(R^{N})₂, and SO₂R^{N};
R³ is C₁₋₆alkylene-X, C₂₋₆alkenylene-X, C₀₋₂alkylene-C₃₋₆carbocycle-C₀₋₂alkylene-X, or Ar, and the alkylene is optionally substituted with OR^{N};
X is H, OC₁₋₃alkyl, C≡CR^{N}; CN, CO₂R^{N}; CON(R^{N})₂, or Ar,
Ar is a 3-10 membered aromatic or non-aromatic monocyclic or polycyclic ring having 0-4 ring heteroatoms selected from N, O, and S, with the proviso that when Ar is a 6-membered aromatic ring, it has 0 or 2-4 ring heteroatoms,
Ar is optionally substituted with C₁₋₃alkyl, C₀₋₂alkylene-CN, CON(R^{N})₂, tetrazole, oxazole, or 1-2 halo;
o is 0 or 1;
R^{z}, when present, is CN, halo, C(O)N(R^{N})₂, C₁₋₆alkyl, C₁₋₆alkoxy, C₁₋₆hydroxyalkyl, or C₁₋₆haloalkyl; and
each R^{N} is independently H, C₁₋₆alkyl, C₁₋₆hydroxyalkyl, or C₁₋₆haloalkyl.

99. The compound or salt of claim 98, wherein R¹ is H.

100. The compound or salt of claim 98 or 99, wherein R^{A} is H.

101. The compound or salt of claim 98 or 99, wherein R^{A} is OC₁₋₆alkylene-N(R^{N})₂ or OC₁₋₆alkylene-OR^{N}.

102. The compound or salt of claim 98 or 99, wherein R^{A} is OR^{N} or N(R^{N})₂.

103. The compound or salt of any one of claims 98 to 102, wherein ring A is phenyl.

104. The compound or salt of any one of claims 98 to 102, wherein ring A is a 6-membered heteroaryl having 1 or 2 nitrogen ring atoms.

105. The compound or salt of claim 104, wherein ring A is pyridyl.

106. The compound or salt of claim 104, wherein ring A is a diazinyl.

107. The compound or salt of claim 106, wherein ring A is pyrimidinyl.

108. The compound or salt of claim 106, wherein ring A is pyrazinyl.

109. The compound or salt of claim 106, wherein ring A is pyradazinyl.

110. The compound or salt of any one of claims 98 to 109, wherein n is 0.

111. The compound or salt of any one of claims 98 to 109, wherein n is 1.

112. The compound or salt of claim 111, wherein R^{B} is C₁₋₆alkyl.

113. The compound or salt of claim 111, wherein R^{B} is C₁₋₆haloalkyl, C₁₋₆hydroxyalkyl, or halo.

114. The compound or salt of claim 111, wherein R^{B} is CO₂R^{N}, N(R^{N})₂, C₀₋₃alkylene-C(O)N(R^{N})₂, or C₀₋₃alkylene-N(R^{N})C(O)R^{N}.

115. The compound or salt of claim 111, wherein R^{B} is C₃₋₆cycloalkyl, Het, or OHet.

116. The compound or salt of claim 115, wherein Het is an aromatic 5-7 membered heterocycle having 1-3 ring heteroatoms.

117. The compound or salt of claim 115, wherein Het is a non-aromatic 4-7 membered heterocycle having 1-3 ring heteroatoms.

118. The compound or salt of any one of claims 115 to 117, wherein Het is unsubstituted.

119. The compound or salt of any one of claims 115 to 117, wherein Het is substituted.

120. The compound or salt of claim 119, wherein Het is a non-aromatic 4-7 membered heterocycle and is substituted with oxo.

121. The compound or salt of claim 119, wherein Het is substituted with C₁₋₆alkyl.

122. The compound or salt of claim 119, wherein Het is substituted with C₁₋₆alkoxy.

123. The compound or salt of claim 119, wherein Het is substituted with C(O)R^{N} or SO₂R^{N}.

124. The compound or salt of any one of claims 98 to 123, wherein R³ is C₁₋₆alkylene-X.

125. The compound or salt of any one of claims 98 to 123, wherein R³ C₂₋₆alkenylene-X or C₀₋₂alkylene-C₃₋₆carbocycle-C₀₋₂alkylene-X.

126. The compound or salt of any one of claims 98 to 125, wherein X is H, OC₁₋₃alkyl, CN, CO₂R^{N}, or CON(R^{N})₂.

127. The compound or salt of any one of claims 98 to 125, wherein X is C≡CR^{N}.

128. The compound or salt of any one of claims 98 to 125, wherein X or R³ is Ar.

129. The compound or salt of claim 128, wherein Ar is 3-10 membered non-aromatic monocyclic or polycyclic ring having 0-4 ring heteroatoms selected from N, O, and S.

130. The compound or salt of claim 128, wherein Ar is a 5-10 membered aromatic monocyclic or polycyclic ring having 0-4 ring heteroatoms selected from N, O, and S.

131. The compound or salt of claim 130, wherein Ar is phenyl.

132. The compound or salt of claim 130, wherein Ar is a 5-10 membered aromatic monocyclic or polycyclic ring having 1-4 ring heteroatoms selected from N, O, and S.

133. The compound or salt of claim 130, wherein Ar is a 5 or 7-10 membered aromatic monocyclic or polycyclic ring having 1-4 ring heteroatoms selected from N, O, and S.

134. The compound or salt of claim 130, wherein Ar is a 6-10 membered aromatic monocyclic or polycyclic ring having 2-4 ring heteroatoms selected from N, O, and S.

135. The compound or salt of claim 130, wherein Ar is phenyl, tetrahydropyran, dihydropyran, tetrahydrofuran, C₃₋₆cycloalkyl, tetrazole, triazole, oxazole, tetrahydroquinoline, N-methyl-tetrahydroisoquinoline, tetrahydrothiopyranyl-dioxide, pyridinone, piperidinone, or oxetanyl.

136. The compound or salt of any one of claims 130 to 135, wherein Ar is unsubstituted.

137. The compound or salt of any one of claims 130 to 135, wherein Ar is substituted, optionally where at least one substituent is meta to point of attachment.

138. The compound or salt of claim 137, wherein Ar is substituted with C₁₋₃alkyl, C₀₋₂alklene-CN, or CON(R^{N})₂.

139. The compound or salt of claim 137 or 138, wherein Ar is substituted with 1 or 2 halo.

140. The compound or salt of claim 139, wherein the halo is fluoro.

141. The compound or salt of any one of claims 88 to 140, wherein o is 0.

142. The compound or salt of any one of claims 88 to 140, wherein o is 1.

143. The compound or salt of claim 142, wherein R^{z} is methyl or fluoro.

144. The compound or salt of any one of claims 88 to 143, wherein each R^{N} is independently H or methyl.

145. The compound or salt of any one of claims 88 to 143, wherein at least one R^{N} is C₁₋₆hydroxyalkyl or C₁₋₆haloalkyl.

146. The compound or salt of claim 88, having a structure as shown in Table C.

147. A compound, or pharmaceutically acceptable salt thereof, having a structure of formula (IV): 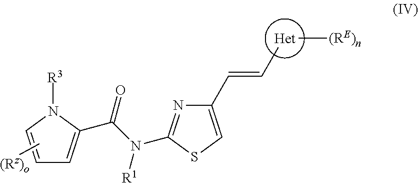
R¹ is H, C₁₋₃alkyl, or SO₂C₁₋₆alkyl;
Het is 3-10 membered aromatic or non-aromatic heterocycle having 1-4 ring heteroatoms selected from N, O, and S;
n is 0, 1, or 2; and
each R^{E}, when present, is independently halo, C₁₋₆alkyl, phenyl, C(O)N(R^{N})₂, CN, C₀₋₆alkylene-OR^{N}, C₀₋₆alkylene-N(R^{N})₂, C₁₋₆haloalkyl, C₁₋₆haloalkoxy, C₃₋₆cycloalkyl, or CO₂R^{N};
wherein when R^{E} is phenyl, it is optionally substituted with 1-2 groups independently selected from halo, C₁₋₆alkyl, CN, C₁₋₆haloalkyl, C₁₋₆haloalkoxy, CO₂R^{N}, CON(R^{N})₂, N(R^{N})COR^{N}, and OR^{N};
R³ is C₁₋₆alkylene-X, C₂₋₆alkenylene-X, Ar, or C₀₋₂alkylene-C₃₋₆carbocycle-C₀₋₂alkylene-X;
X is H, OC₁₋₃alkyl, C≡CR^{N}; CN, CO₂R^{N}; CON(R^{N})₂, or Ar,
Ar is a 3-10 membered aromatic or non-aromatic ring having 0-4 ring heteroatoms selected from N, O, and S, with the proviso that when Ar is a 6-membered aromatic ring, it has 0 or 2-4 ring heteroatoms;
Ar is optionally substituted with C₁₋₃alkyl, C₀₋₂alklene-CN, CON(R^{N})₂, tetrazole, oxazole, or 1-2 halo;
o is 0 or 1;
R^{z}, when present, is CN, halo, C(O)N(R^{N})₂, C₁₋₆alkyl, C₁₋₆alkoxy, C₁₋₆hydroxyalkyl, or C₁₋₆haloalkyl; and
each R^{N} is independently H, C₁₋₆alkyl, C₁₋₆hydroxyalkyl, or C₁₋₆haloalkyl.

148. The compound or salt of claim 147, wherein R¹ is H.

149. The compound or salt of claim 147 or 148, wherein Het is a 3-10 membered non-aromatic heterocycle having 1-4 ring heteroatoms selected from N, O, and S.

150. The compound or salt of claim 149, wherein Het is tetrahydropyran.

151. The compound or salt of claim 147 or 148, wherein Het is a 5-10 membered aromatic heterocycle having 1-4 ring heteroatoms selected from N, O, and S.

152. The compound or salt of claim 151, wherein oxazole.

153. The compound or salt of claim 151, wherein Het is imidazole.

154. The compound or salt of claim 151, wherein Het is diazinyl.

155. The compound or salt of claim 154, wherein the diazinyl is pyrimidinyl.

156. The compound or salt of claim 154, wherein the diazinyl is pyrazinyl.

157. The compound or salt of claim 154, wherein the diazinyl is pyradazinyl.

158. The compound or salt of claim 147 or 148, wherein Het is isoxazole, morpholine, tetrahydroquinoline, oxazolidinone, piperidinone, or dihydrooxazole.

159. The compound or salt of any one of claims 147 to 158, wherein n is 0.

160. The compound or salt of any one of claims 147 to 158, wherein n is 1.

161. The compound or salt of any one of claims 147 to 158, wherein n is 2.

162. The compound or salt of claim 160 or 161, wherein at least one R^{E} is halo.

163. The compound or salt of claim 162, wherein at least one R^{E} is fluoro.

164. The compound or salt of any one of claims 160 to 163, wherein at least one R^{E} is C₁₋₆alkyl or C(O)N(R^{N})₂.

165. The compound or salt of any one of claims 160 to 164, wherein at least one R^{E} is C₀₋₆alkylene-OR^{N} or C₀₋₆alkylene-N(R^{N})₂.

166. The compound or salt of any one of claims 160 to 165, wherein at least one R^{E} is phenyl.

167. The compound or salt of claim 166, wherein the phenyl is unsubstituted.

168. The compound or salt of claim 166, wherein the phenyl is substituted with 1 substituent selected from halo, C₁₋₆haloalkyl, C₁₋₆haloalkoxy, CON(R^{N})₂, N(R^{N})COR^{N} and OR^{N}.

169. The compound or salt of any one of claims 147 to 168, wherein R³ is C₁₋₆alkylene-X, optionally CH₂X.

170. The compound or salt of any one of claims 147 to 168, wherein R³ is C₂₋₆alkenylene-X or C₀₋₂alkylene-C₃₋₆carbocycle-C₀₋₂alkylene-X.

171. The compound or salt of any one of claims 147 to 170, wherein X is H, OC₁₋₃alkyl, CN, CO₂R^{N}, or CON(R^{N})₂.

172. The compound or salt of any one of claims 147 to 170, wherein X is C≡CR^{N}.

173. The compound or salt of any one of claims 147 to 170, wherein X or R³ is Ar.

174. The compound or salt of claim 173, wherein Ar is 3-10 membered non-aromatic monocyclic or polycyclic ring having 0-4 ring heteroatoms selected from N, O, and S.

175. The compound or salt of claim 173, wherein Ar is a 5-10 membered aromatic monocyclic or polycyclic ring having 0-4 ring heteroatoms selected from N, O, and S.

176. The compound or salt of claim 173, wherein Ar is phenyl, optionally where R³ is CH₂-phenyl.

177. The compound or salt of claim 173, wherein Ar is a 5-10 membered aromatic monocyclic or polycyclic ring having 1-4 ring heteroatoms selected from N, O, and S.

178. The compound or salt of claim 177, wherein Ar is a 5 or 7-10 membered aromatic monocyclic or polycyclic ring having 1-4 ring heteroatoms selected from N, O, and S.

179. The compound or salt of claim 177, wherein Ar is a 6-10 membered aromatic monocyclic or polycyclic ring having 2-4 ring heteroatoms selected from N, O, and S.

180. The compound or salt of claim 173, wherein Ar is phenyl, tetrahydropyran, dihydropyran, tetrahydrofuran, C₃₋₆cycloalkyl, tetrazole, triazole, oxazole, tetrahydroquinoline, N-methyl-tetrahydroisoquinoline, tetrahydrothiopyranyl-dioxide, pyridinone, piperidinone, or oxetanyl.

181. The compound or salt of any one of claims 173 to 180, wherein Ar is unsubstituted.

182. The compound or salt of any one of claims 173 to 180, wherein Ar is substituted, optionally where, when Ar is phenyl, the substitution is meta to the point of attachment of the phenyl.

183. The compound or salt of claim 182, wherein Ar is substituted with C₁₋₃alkyl, C₀₋₂alklene-CN, or CON(R^{N})₂.

184. The compound or salt of claim 182 or 183, wherein Ar is substituted with 1 or 2 halo.

185. The compound or salt of claim 184, wherein the halo is fluoro.

186. The compound or salt of claim 147, having a structure as shown in Table D.

187. A compound, or pharmaceutically acceptable salt thereof, as listed in Table E.

188. A pharmaceutical composition comprising the compound or salt of any one of claims 1 to 187 and a pharmaceutically acceptable excipient.

189. A method of inhibiting protein secretion in a cell comprising contacting the cell with the compound or salt of any one of claims 1 to 187 in an amount effective to inhibit secretion.

190. The method of claim 189, wherein the protein is a checkpoint protein.

191. The method of claim 189, wherein the protein is a cell-surface protein, endoplasmic reticulum associated protein, or secreted protein involved in regulation of anti-tumor immune response.

192. The method of claim 189, wherein the protein is at least one of PD-1, PD-L1, TIM-1, LAG-3, CTLA4, BTLA, OX-40, B7H1, B7H4, CD137, CD47, CD96, CD73, CD40, VISTA, TIGIT, LAIR1, CD160, 2B4, TGFRβ and combinations thereof.

193. The method of claim 189, wherein the protein is selected from the group consisting of HER3, TNFα, IL2, and PD1.

194. The method of any one of claims 189 to 193, wherein the contacting comprising administering the compound to a subject in need thereof.

195. A method for treating inflammation in a subject comprising administering to the subject a therapeutically effective amount of the compound or salt of any one of claims 1 to 187.

196. A method for treating cancer in a subject comprising administering to the subject a therapeutically effective amount of the compound or salt of any one of claims 1 to 187.

197. The method of claim 196, wherein the cancer is melanoma, multiple myeloma, prostate cancer, lung cancer, pancreatic cancer, squamous cell carcinoma, leukemia, lymphoma, a neuroendocrine tumor, bladder cancer, or colorectal cancer.

198. The method of claim 196, wherein the cancer is selected from the group consisting of prostate, lung, bladder, colorectal, and multiple myeloma.

199. The method of claim 196, wherein the cancer is non-small cell lung carcinoma, squamous cell carcinoma, leukemia, acute myelogenous leukemia, chronic myelogenous leukemia, lymphoma, NPM/ALK-transformed anaplastic large cell lymphoma, diffuse large B cell lymphoma, neuroendocrine tumors, breast cancer, mantle cell lymphoma, renal cell carcinoma, rhabdomyosarcoma, ovarian cancer, endometrial cancer, small cell carcinoma, adenocarcinoma, gastric carcinoma, hepatocellular carcinoma, pancreatic cancer, thyroid carcinoma, anaplastic large cell lymphoma, hemangioma, or head and neck cancer.

200. The method of claim 196, wherein the cancer is a solid tumor.

201. The method of claim 196, wherein the cancer is head and neck cancer, squamous cell carcinoma, gastric carcinoma, or pancreatic cancer.

202. A method for treating an autoimmune disease in a subject comprising administering to the subject a therapeutically effective amount of the compound or salt of any one of claims 1 to 187.

203. The method of claim 202, wherein the autoimmune disease is psoriasis, dermatitis, systemic scleroderma, sclerosis, Crohn's disease, ulcerative colitis; respiratory distress syndrome, meningitis; encephalitis; uveitis; colitis; glomerulonephritis; eczema, asthma, chronic inflammation; atherosclerosis; leukocyte adhesion deficiency; rheumatoid arthritis; systemic lupus erythematosus (SLE); diabetes mellitus; multiple sclerosis; Reynaud's syndrome; autoimmune thyroiditis; allergic encephalomyelitis; Sjorgen's syndrome; juvenile onset diabetes; tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia; myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia.

204. A method for the treatment of an immune-related disease in a subject comprising administering to the subject a therapeutically effective amount of the compound or salt of any one of claims 1 to 187.

205. The method of claim 204, wherein the immune-related disease is rheumatoid arthritis, lupus, inflammatory bowel disease, multiple sclerosis, or Crohn's disease.

206. A method for treating neurodegenerative disease in a subject comprising administering to the subject a therapeutically effective amount of the compound or salt of any one of claims 1 to 187.

207. The method of claim 206, wherein the neurodegenerative disease is multiple sclerosis.

208. A method for treating an inflammatory disease in a subject comprising administering to the subject a therapeutically effective amount of the compound or salt of any one of claims 1 to 187.

209. The method of claim 208, wherein the inflammatory disease is bronchitis, conjunctivitis, myocarditis, pancreatitis, chronic cholecstitis, bronchiectasis, aortic valve stenosis, restenosis, psoriasis or arthritis.
